(19) United States
(12) Patent Application Publication
(10) Pub. No.: US 2020/0206233 A1
(43) Pub. Date: Jul. 2, 2020

(54) HETEROCYCLIC COMPOUNDS AS MUTANT IDH INHIBITORS

(71) Applicant: INTEGRAL BIOSCIENCES PRIVATE LIMITED, Noida (IN)

(72) Inventors: Sarvajit CHAKRAVARTY, Edmond, OK (US); Dhananjay PENDHARKAR, Noida (IN); Sreekanth A. RAMACHANDRAN, Noida (IN); Chandramohan BATHULA, Noida (IN); Sanjeev SONI, Noida (IN); Vivek KUMAR, Delhi (IN); Uzma SAEED, New Delhi (IN); Abhinandan Kumar DANODIA, Greater Noida (IN); Ankesh SHARMA, Noida (IN); Pradeep S. JADHAVAR, Greater Noida (IN)

(21) Appl. No.: 16/729,696

(22) Filed: Dec. 30, 2019

(30) Foreign Application Priority Data
Dec. 31, 2018 (IN) 201811049920

(51) Int. Cl. A61K 31/53; C07D 413/14; C07D 413/04; C07D 471/04; A61K 45/06

(52) U.S. Cl. CPC A61K 31/53; C07D 413/14; A61K 45/06; C07D 471/04; C07D 413/04

(57) ABSTRACT

The present disclosure relates generally to compounds useful in treatment of conditions associated with mutant isocitrate dehydrogenase (mt-IDH), particularly mutant IDH1 enzymes. Specifically, the present invention discloses compound of formula (IA), which exhibits inhibitory activity against mutant IDH1 enzymes. Method of treating conditions associated with excessive activity of mutant IDH1 enzymes with such compound is disclosed. Uses thereof, pharmaceutical composition, and kits are also disclosed.

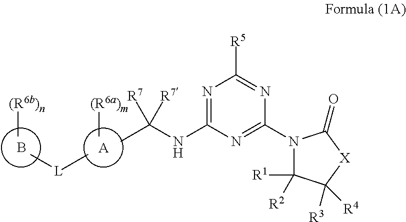

### CROSS REFERENCE TO RELATED APPLICATION

This application claims the priority benefit of INDIAN Provisional Patent Application No. 201811049920, filed Dec. 31, 2018, the disclosures of which is incorporated herein by reference in its entireties.

### FIELD OF THE INVENTION

The present invention generally relates to compounds possessing inhibitory activity of mutant isocitrate dehydrogenase (mt-IDH) proteins with neomorphic activity useful in the treatment of proliferative disorders, such as cancer. The invention also provides method of synthesis of said compounds, method of using said compounds, pharmaceutical compositions comprising said compounds and method of using thereof.

### BACKGROUND OF THE INVENTION

Isocitrate dehydrogenase (IDH) is a family of enzymes found in cellular metabolism. They are NADP⁺/NAD⁺ and metal dependent oxidoreductases of the enzyme class EC 1.1.1.42. IDH catalyzes the oxidative decarboxylation of isocitrate, producing alpha-ketoglutarate (α-ketoglutarate) and CO₂. IDH exists in three isoforms in humans i.e. IDH1, IDH2 and IDH3, wherein IDH3 catalyzes the third step of the citric acid cycle while converting NAD⁺ to NADH in the mitochondria. The isoforms IDH1 and IDH2 catalyze the same reaction outside the context of the citric acid cycle and use NADP⁺ as a cofactor instead of NAD⁺. IDHs localize to the cytosol as well as the mitochondrion and peroxisome.

The wild type proteins catalyze the oxidative decarboxylation of isocitrate to α-ketoglutarate, generating carbon dioxide and NADPH/NADH in the process. They are also known to convert oxalosuccinate into α-ketoglutarate.

Mutations in IDH1 (cytosolic) and IDH2 (mitochondrial) have been identified in multiple cancer types including, but not limited to glioma, glioblastoma multiforme, paraganglioma, supratentorial primordial neuroectodermal tumors, acute myeloid leukemia (AML), prostate cancer, thyroid cancer, colon cancer, chondrosarcoma, cholangiocarcinoma, peripheral T-cell lymphoma, and melanoma. (L. Dang et al., Trends Mol. Med., 2010, 16, 387; T. Shibata et al., Am. J. Pathol., 2011, 178(3), 1395; Gaal et al., J. Clin. Endocrinol. Metab. 2010, 95(3), 1274; Balss et al., Acta Neuropathol., 2008, 116, 597) The mutations have been found at or near key residues in the active site: G97D, R100Q, R132H, H133Q, and A134D for IDH1, and R140 and R172 for IDH2. (L. Dang et al., Nature, 2009, 462, 739; L. Sellner et al., Eur. J. Haematol., 2010, 85, 457)

These mutant forms of IDH are believed to have a neomorphic activity, reducing α-ketoglutarate to 2-hydroxyglutarate (2-HG). (P. S. Ward et al., Cancer Cell, 2010, 17, 225) In general, production of 2-HG is enantiospecific, resulting in generation of the D-enantiomer (also known as the R enantiomer or R-2-HG). Normal cells generally have low native levels of 2-HG, whereas cells harboring these mutations in IDH1 or IDH2 show significantly elevated levels of 2-HG. 2-HG production is believed to contribute to the formation and progression of cancer. (Dang, et al. 2009 Nature 462:739-44.) High levels of 2-HG have also been detected in tumors harboring the mutations. High levels of 2-HG have been detected in the plasma of patients with mutant IDH containing AML. (S. Gross et al., J. Exp. Med., 2010, 207(2), 339)

Mutations in IDH1 have been associated with multiple cancers and patients having these disorders often have increased levels of 2-HG in their urine, plasma or cerebrospinal fluid. (M. Kranendijk et al., Science, 2010, 330, 336)

There is a an urgent and growing need for small molecule inhibitors of mutant IDH enzymes, or more specifically mutant IDH1 enzymes, for the treatment of diseases and disorders associated with this enzymes. Therefore, the present invention provides inhibitors of mutant isocitrate dehydrogenase (mt-IDH1).

### SUMMARY OF THE INVENTION

In one aspect, the present invention provides a compound of formula (IA):

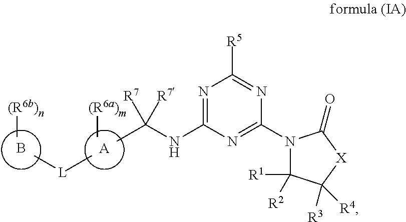

or a salt, polymorph, solvate, enantiomer, stereoisomer or tautomer thereof, wherein X, A, B, L, R¹, R², R³, R⁴, R⁵, R^{6a}, R^{6b}, R⁷, R^{7′}, m and n are as detailed herein.

In some aspects, the compound of formula (IA) or a salt, polymorph, solvate, enantiomer, stereoisomer or tautomer thereof, is any of the compounds of formula (I), (Ia-1) to (Ia-14), (Ib-1) to (Ib-11) or a salt, polymorph, solvate, enantiomer, stereoisomer or tautomer thereof, as detailed herein.

In some aspects, the compound of formula (IA) or a salt, polymorph, solvate, enantiomer, stereoisomer or tautomer thereof, is a compound of formula (II), (IIa-1) to (IIa-8) or a salt, polymorph, solvate, enantiomer, stereoisomer or tautomer thereof, as detailed herein.

In some aspects, the compound of formula (IA) or a salt, polymorph, solvate, enantiomer, stereoisomer or tautomer thereof, is a compound of formula (III), (IIIa-1) to (IIIa-8) or a salt, polymorph, solvate, enantiomer, stereoisomer or tautomer thereof, as detailed herein.

In some aspects, the compound of formula (IA) or a salt, polymorph, solvate, enantiomer, stereoisomer or tautomer thereof, is any of the compounds of formula (IV), (IVa-1) to (IVa-7) or a salt, polymorph, solvate, enantiomer, stereoisomer or tautomer thereof, as detailed herein.

In some aspects, the compound of formula (IA) or a salt, polymorph, solvate, enantiomer, stereoisomer or tautomer thereof, is a compound of formula (V) or a salt, polymorph, solvate, enantiomer, stereoisomer or tautomer thereof, as detailed herein.

In some aspects, the compound of formula (IA) or a salt, polymorph, solvate, enantiomer, stereoisomer or tautomer thereof, is a compound of formula (VI) or a salt, polymorph, solvate, enantiomer, stereoisomer or tautomer thereof, as detailed herein.

In some aspects, the present invention provides method of treating a disease or disorder associated with this mutant IDH enzymes, more specifically mutant IDH1 enzymes in an individual in need thereof, wherein the method comprises administering to the individual an effective amount of a compound of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)), or a salt, polymorph, solvate, enantiomer, stereoisomer or tautomer thereof.

In some aspects, the present invention provides method of treating cancer in an individual in need thereof, wherein the method comprises administering to the individual an effective amount of a compound of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)), or a salt, polymorph, solvate, enantiomer, stereoisomer or tautomer thereof.

In some aspects, the present invention provides method of inhibiting mutant IDH1 in an individual in need thereof, wherein the method comprises administering to the individual an effective amount of a compound of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)), or a salt, polymorph, solvate, enantiomer, stereoisomer or tautomer thereof.

In some aspects, the present invention provides method of treating a disease or disorder associated with this mutant IDH enzymes, or more specifically mutant IDH1 enzymes in an individual in need thereof, wherein the method comprises administering to the individual an effective amount of a compound of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)), or a salt, polymorph, solvate, enantiomer, stereoisomer or tautomer thereof in combination with other therapeutic agents.

In some aspects, the present invention also provides pharmaceutical compositions, comprising a compound of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)) and at least one pharmaceutically acceptable excipient.

In some aspects, the present invention provides method of treating a disease or disorder associated with this mutant IDH enzymes, or more specifically mutant IDH1 enzymes in an individual in need thereof, wherein the method comprises administering to the individual a pharmaceutical composition comprising an effective amount of a compound of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)), or a salt, polymorph, solvate, enantiomer, stereoisomer or tautomer thereof.

In some aspects, the present invention provides uses of the compound of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)), or a salt, polymorph, solvate, enantiomer, stereoisomer or tautomer thereof in the manufacture of the medicament for treatment of a disease or disorder associated with this mutant IDH enzymes, or more specifically mutant IDH1 enzymes.

In some aspects, the present invention provides processes for preparing compounds and intermediates thereof disclosed in the present invention.

### DETAILED DESCRIPTION OF THE INVENTION

### Definitions

“Alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Alkynyl” as used herein refers to an unsaturated linear or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Aryl” refers to and includes polyunsaturated aromatic hydrocarbon groups. Aryl may contain additional fused rings (e.g., from 1 to 3 rings), including additionally fused aryl, heteroaryl, cycloalkyl, and/or heterocyclyl rings. In one variation, the aryl group contains from 6 to 14 annular carbon atoms. Examples of aryl groups include, but are not limited to, phenyl, naphthyl, biphenyl, and the like.

“Carbonyl” refers to the group C═O.

“Cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to imidazolyl, pyrrolyl, pyrazolyl, 1,2,4-triazolyl, thiophenyl, furanyl, thiazolyl, isothiazolyl, 1,3,4-thiadiazolyl oxazolyl, isoxazolyl, 1,3,4-oxadiazolyl, pyridyl, pyrimidyl, pyridazinyl, pyrazinyl, indolyl, indazolyl, benzoimidazolyl, pyrrolopyridinyl, pyrrolopyridazinyl, pyrrolopyrimidinyl, pyrazolopyridinyl, pyrazolopyrimidinyl, imidazopyridinyl, purinyl, benzofuranyl, furopyridinyl, benzooxazolyl, benzothiophenyl, benzothiazolyl, oxazolopyridinyl, thiazolopyridinyl, thienopyridinyl, quinolinyl, quinolonyl, naphthyridinyl, quinazolinyl, pyridopyrimidinyl, cinnolinyl or pyridopyridazinyl and the like.

“Heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, aziridinyl, azetidinyl, oxetanyl, morpholinyl, thiomorpholinyl, azepanyl tetrahydropyranyl, dihydropyranyl, piperidinyl, piperazinyl, pyrrolidinyl, thiazolinyl, thiazolidinyl, tetrahydrofuranyl, tetrahydrothiophenyl, and the like.

“Oxo” refers to the moiety ═O.

“IDH” refers to Isocitrate dehydrogenases, which includes IDH1 and IDH2. IDH refers herein specifically to mutant IDH, more specifically mutant IDH1.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

A “medicament” or “pharmaceutical composition” refer to an pharmaceutical formulation in administrable form comprising at least one pharmaceutically active ingredient and one or more pharmaceutically acceptable carrier.

A “pharmaceutically acceptable carrier” or “pharmaceutically acceptable excipient” refer to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. In reference to cancers or other unwanted cell proliferation, beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying occurrence and/or recurrence, such as of unwanted cell proliferation.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations, in the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective dosage can be administered in one or more administrations. For purposes of this disclosure, an effective dosage of compound or a salt thereof, or pharmaceutical composition is an amount sufficient to accomplish prophylactic or therapeutic treatment either directly or indirectly. It is intended and understood that an effective dosage of a compound or salt thereof, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer). In some embodiments, the individual is at an advanced stage of a proliferative disease (such as an advanced cancer).

Reference to “about” a value or parameter herein includes (and describes) embodiments that are directed to that value or parameter per se. For example, description referring to “about X” includes description of “X”.

It is understood that aspects and variations described herein also include “consisting” and/or “consisting essentially of” aspects and variations.

### Compounds

In one aspect, provided is a compound of the formula (IA):

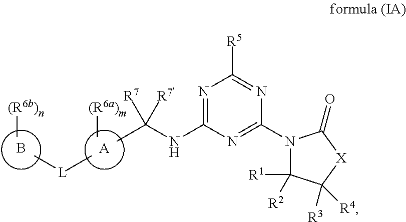

or a salt, polymorph, solvate, enantiomer, stereoisomer or tautomer thereof, wherein

wherein,

X is O, S, NR^{a} or CR^{b}R^{c};

A is C₆-C₁₀ aryl, 5- to 10-membered heteroaryl, C₃-C₈ cycloalkyl or 3- to 10-membered heterocyclyl, wherein each of which is optionally substituted by R^{6a};

B is hydrogen, C₆-C₁₀ aryl, 5- to 10-membered heteroaryl, C₃-C₈ cycloalkyl or 3- to 10-membered heterocyclyl, wherein each of which is optionally substituted by R^{6b};

L is a bond, —O—, —(CH₂)₁₋₃—, —NH—, —NCH₃—, —SO₂—, —C(O)—, —CH₂—O—, —S—, —CR^{b}R^{c}—, —C(O)NH— or —NHC(O)—;

R^{a} is hydrogen or C₁-C₆ alkyl optionally substituted by oxo, —OH or halogen;

R^{b} and R^{c} are independently hydrogen, halogen, —CN, C₁-C₆ alkyl, C₃-C₆ cycloalkyl, or —(C₁-C₃ alkylene)(C₃-C₆ cycloalkyl);

R¹ is hydrogen, halogen or C₁-C₆ alkyl;

R² is hydrogen, halogen, C₁-C₆ alkoxy, C₁-C₆ haloalkoxy, —C(O)OR^{2a}, C₃-C₆ cycloalkyl, 3- to 6-membered heterocyclyl, C₆ aryl, 5- to 6-membered heteroaryl, —(C₁-C₃ alkylene)C₆ aryl or C₁-C₆ alkyl optionally substituted by oxo, halogen, —OR^{2a} or —NR^{2a}R^{2b}, wherein C₃-C₆ cycloalkyl, 3- to 6-membered heterocyclyl, C₆ aryl, 5- to 6-membered heteroaryl, —(C₁-C₃ alkylene)C₆ aryl of R² optionally substituted by C₁-C₆ alkyl;

or R¹ and R² are taken together with the atom to which they are attached to form a C₃-C₆ cycloalkyl or 3- to 6-membered heterocyclyl, each of which is optionally substituted by oxo, —OH, halogen, —NH₂, or C₁-C₆ alkyl optionally substituted by oxo, —OH, halogen or —NH₂;

R^{2a} and R^{2b} are independently hydrogen or C₁-C₆ alkyl;

R³ and R⁴ are independently hydrogen, halogen, or C₁-C₆ alkyl optionally substituted by oxo, —OH or halogen;

or R³ and R⁴ are taken together with the atom to which they are attached to form a C₃-C₆ cycloalkyl or 3- to 6-membered heterocyclyl, each of which is optionally substituted by oxo, —OH, -halogen, —NH₂, or C₁-C₆ alkyl optionally substituted by oxo, —OH, halogen or —NH₂;

R⁵ is hydrogen, C₁-C₆ alkyl, C₂-C₆ alkenyl, C₂-C₆ alkynyl, C₃-C₆ cycloalkyl, 3- to 6-membered heterocyclyl, C₆ aryl, 5- to 6-heteroaryl, —CN, halogen, C₁-C₆ alkoxy, C₁-C₆ haloalkoxy, —OR¹⁰, —SR¹⁰, —S(O)₂R¹⁰, —S(O)₂NR¹¹R¹², —NR¹⁰S(O)₂R¹¹, —NR¹¹R¹², —C(O)R¹⁰, —NR¹⁰C(O)R¹¹, —NR¹⁰C(O)NR¹¹R¹², —C(O)OR¹⁰, —C(O)ONR¹¹R¹², —C(O)NR¹¹R¹², wherein each of which is optionally substituted by R⁸;

each R^{6a} and R^{6b} is independently oxo, C₁-C₆ alkyl, C₂-C₆ alkenyl, C₂-C₆ alkynyl, C₃-C₆ cycloalkyl, 3- to 6-membered heterocyclyl, C₆ aryl, —CN, halogen, C₁-C₆ alkoxy, C₁-C₆ haloalkoxy, C₁-C₆ haloalkyl, —OR¹³, —SR¹³, —S(O)₂R¹³, —S(O)₂NR¹⁴R¹⁵, —NR¹³S(O)₂R¹⁴, —NR¹⁴R¹⁵, —C(O)R¹³, —NR¹³C(O)R¹⁴, —NR¹³C(O)NR¹⁴R¹⁵, —C(O)OR¹³, —C(O)ON¹⁴R¹⁵, —C(O)NR¹⁴R¹⁵, —(C₁-C₃ alkylene)OR¹³, —(C₁-C₃ alkylene)SR¹³, —(C₁-C₃ alkylene)S(O)₂R¹³, —(C₁-C₃ alkylene)S(O)₂NR¹⁴R¹⁵, —(C₁-C₃ alkylene)NR¹³S(O)₂R¹⁴, —(C₁-C₃ alkylene)NR¹⁴R¹⁵, —(C₁-C₃ alkylene)C(O)R¹³, —(C₁-C₃ alkylene)NR¹³C(O)R¹⁴, —(C₁-C₃ alkylene)NR¹³C(O)NR¹⁴R¹⁵, —(C₁-C₃ alkylene)C(O)OR¹³, —(C₁-C₃ alkylene)C(O)ONR¹⁴R¹⁵, —(C₁-C₃ alkylene)(C₃-C₈ cycloalkyl) or —(C₁-C₃ alkylene)(3- to 10-membered heterocyclyl); wherein each of R^{6a} and R^{6b} is independently optionally substituted by oxo, C₂-C₆ alkenyl, C₂-C₆ alkynyl, —CN, halogen, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶, C₁-C₆ alkyl optionally substituted by oxo, OH, halogen or NH₂;

R⁷ and R^{7′} are independently hydrogen, C₃-C₆ cycloalkyl or C₁-C₆ alkyl optionally substituted by halogen or —OH;

R⁷ and R^{7′} are taken together with the atom to which they are attached to form a C₃-C₆ cycloalkyl;

R⁸ is halogen, oxo, C₂-C₆ alkenyl, C₂-C₆ alkynyl, C₃-C₆ cycloalkyl 3- to 6-membered heterocyclyl, —CN, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶ or C₁-C₆ alkyl optionally substituted by oxo, —OH, halogen or NH₂;

each R¹⁰, R¹¹ and R¹² is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹⁰, R¹¹ and R¹² is independently optionally substituted by oxo, C₂-C₆ alkenyl, C₂-C₆ alkynyl, —CN, halogen, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶ or C₁-C₆ alkyl optionally substituted by oxo, OH, halogen or NH₂;

or R¹¹ and R¹² are taken together with the atom to which they attached to form a 3- to 6-membered heterocyclyl optionally substituted by oxo, OH, halogen, NH₂, or C₁-C₆ alkyl optionally substituted by oxo, OH, halogen or NH₂;

each R¹³, R¹⁴ and R¹⁵ is independently hydrogen, C₁-C₆ alkyl, C₂-C₆ alkenyl, C₂-C₆ alkynyl, C₃-C₆ cycloalkyl, 3- to 6-membered heterocyclyl, —(C₁-C₃ alkylene)C₃-C₆ cycloalkyl or —(C₁-C₃ alkylene) 5- to 6-heteroaryl, wherein each of R¹³, R¹⁴ and R¹⁵ is independently optionally substituted by oxo, C₂-C₆ alkenyl, C₂-C₆ alkynyl, —CN, halogen, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶ or C₁-C₆ alkyl optionally substituted by oxo, OH, halogen or NH₂;

or R¹⁴ and R¹⁵ are taken together with the atom to which they attached to form a 3- to 6-membered heterocyclyl optionally substituted by oxo, OH or halogen, or C₁-C₆ alkyl optionally substituted by oxo, OH, halogen or NH₂;

each R¹⁶, R¹⁷ and R¹⁸ is independently hydrogen, C₂-C₆ alkenyl, C₂-C₆ alkynyl, or C₁-C₆ alkyl optionally substituted by oxo, OH, halogen or NH₂;

or R¹⁷ and R¹⁸ are taken together with the atom to which they attached to form a 3- to 6-membered heterocyclyl optionally substituted by oxo, OH, halogen or NH₂, or C₁-C₆ alkyl optionally substituted by oxo, OH, halogen or NH₂; and

m and n are independently 0, 1, 2, 3 or 4.

In some embodiments of a compound of formula (IA), X is O. In some embodiments of a compound of formula (IA), X is S. In some embodiments of a compound of formula (IA), X is NR^{a}. In some embodiments of a compound of formula (IA), X is CR^{b}R^{c}.

In some embodiments of a compound of formula (IA), X is NR^{a}. In some embodiments of a compound of formula (IA), R^{a} is hydrogen. In some embodiments of a compound of formula (IA), R^{a} is C₁-C₆ alkyl optionally substituted by oxo, —OH or halogen. In some embodiments of a compound of formula (IA), R^{a} is —CH₃.

In some embodiments of a compound of formula (IA), X is CR^{b}R^{c}. In some embodiments of a compound of formula (IA), R^{b} and R^{c} are independently selected from hydrogen, halogen, —CN, C₁-C₆ alkyl or C₃-C₆ cycloalkyl. In some embodiments of a compound of formula (IA), R^{b} or R^{c} is methyl or cycloproyl. In some embodiments of a compound of formula (IA), R^{b} and R^{c} are hydrogen. In some embodiments of a compound of formula (IA), one of the R^{b} and R^{c} is —CN; and the other one of R^{b} and R^{c} is cycloproyl.

In some embodiments of a compound of formula (IA), A is C₆-C₁₀ aryl, 5- to 10-membered heteroaryl, C₃-C₈ cycloalkyl or 3- to 10-membered heterocyclyl, wherein each of which is optionally substituted by R^{6a}. In some embodiments of a compound of formula (IA), A is C₆-C₁₀ aryl optionally substituted by R^{6a}. In some embodiments of a compound of formula (IA), A is phenyl optionally substituted by R^{6a}. In some embodiments of a compound of formula (IA), A is an unsubstituted phenyl. In some embodiments of a compound of formula (IA), A is phenyl substituted by halogen, —CN, C₆-aryl, C₁-C₆ alkyl, —OR¹³. In some embodiments of a compound of formula (IA), A is phenyl substituted by —Cl, —F, methyl, —OCH₃, —CN, —OCF₃ and phenyl.

In some embodiments of a compound of formula (IA), A is naphthyl optionally substituted by R^{6a}. In some embodiments of a compound of formula (IA), A is naphthyl optionally substituted by —OR¹³. In some embodiments of a compound of formula (IA), A is naphthyl optionally substituted by halogen. In some embodiments of a compound of formula (IA), A is naphthyl substituted by —Cl or —OCH₃.

In some embodiments of a compound of formula (IA), A is 5- to 10-membered heteroaryl optionally substituted by R^{6a}. In some embodiments of a compound of formula (IA), A is 5-membered heteroaryl optionally substituted by R^{6a}. In some embodiments of a compound of formula (IA), A is 6-membered heteroaryl optionally substituted by R^{6a}. In some embodiments of a compound of formula (IA), A is 9-membered bicyclic heteroaryl optionally substituted by R^{6a}, in which any one ring or both rings may be substituted by same or different R^{6a}. In some embodiments of a compound of formula (IA), A is 10-membered bicyclic heteroaryl optionally substituted by R^{6a}, in which any one ring or both rings may be substituted by same or different R^{6a}.

In some embodiments of a compound of formula (IA), A is 5-membered heteroaryl selected from imidazolyl, pyrrolyl, pyrazolyl, triazolyl, thiophenyl, furanyl, thiazolyl, isothiazolyl, 1,3,4-thiadiazolyl, oxazolyl, isoxazolyl or 1,3,4-oxadiazolyl, wherein each of which is optionally substituted by R^{6a}, wherein R^{6a} is selected from C₁-C₆ alkyl, —CN, halogen, C₁-C₆ alkoxy, C₁-C₆ haloalkoxy or C₁-C₆ haloalkyl.

In some embodiments of a compound of formula (IA), A is imidazolyl, triazolyl, oxadiazolyl or isoxazolyl optionally substituted by methyl or —F,

In some embodiments of a compound of formula (IA), A is 6-membered heteroaryl selected from pyridyl, pyrimidyl, pyridazinyl or pyrazinyl, wherein each of which is optionally substituted by R^{6a} wherein R^{6a} is selected from oxo, C₁-C₆ alkyl, —CN, halogen, C₁-C₆ alkoxy, C₁-C₆ haloalkoxy or C₁-C₆ haloalkyl. In some embodiments, R^{6a} is oxo, —CH₃, —OCH₃ or —Cl.

In some embodiments of a compound of formula (IA), A is pyridyl or pyrimidyl optionally substituted with —CH₃ or —OCH₃.

In some embodiments of a compound of formula (IA), A is 9-membered heteroaryl selected from, but not limited to indolyl, indazolyl, benzoimidazolyl, pyrrolopyridinyl, pyrrolopyridazinyl, pyrrolopyrimidinyl, pyrazolopyridinyl, pyrazolopyrimidinyl, imidazopyridinyl, purinyl, benzofuranyl, furopyridinyl, benzooxazolyl, benzothiophenyl, benzothiazolyl, oxazolopyridinyl, thiazolopyridinyl or thienopyridinyl, wherein each of which is optionally substituted by R^{6a}.

In some embodiments of a compound of formula (IA), A is 10-membered heteroaryl selected from, but not limited to quinolinyl, quinolonyl, naphthyridinyl, quinazolinyl, pyridopyrimidinyl, cinnolinyl or pyridopyridazinyl, wherein each of which is optionally substituted by R^{6a}. In some embodiments of a compound of formula (IA), 10-membered heteroaryl of A is quinolinyl substituted by oxo, methyl, —CN, —Cl, —F, —Br, —OCH₃, —OCF₃, —CF₃, cyclopropyl or —OH. In some embodiments of a compound of formula (IA), 10-membered heteroaryl of A is quinolonyl substituted by methyl, —CN, —Cl, —F, —Br, —OCH₃, —OCF₃, —CF₃ or cyclopropyl.

In some embodiments of a compound of formula (IA), A is 3- to 10-membered heterocyclyl optionally substituted by R^{6a}. In some embodiments of a compound of formula (IA), A is 3- to 10-membered heterocyclyl selected from aziridinyl, azetidinyl, pyrrolidinyl, piperidinyl, piperazinyl, morpholinyl, thiomorpholinyl, pyrrolidinyl or azepanyl, wherein each of which is optionally substituted by R^{6a}. In some embodiments, heterocyclyl ring may be fused with aryl or heteraryl ring to form biycylic ring which is optionally substituted R^{6a}, in which one ring or both rings may be substituted by the same or different R^{6a}. In some embodiments, heterocyclyl ring may be saturated or partially unsaturated.

In some embodiments of a compound of formula (IA), A is selected from the group consisting of:

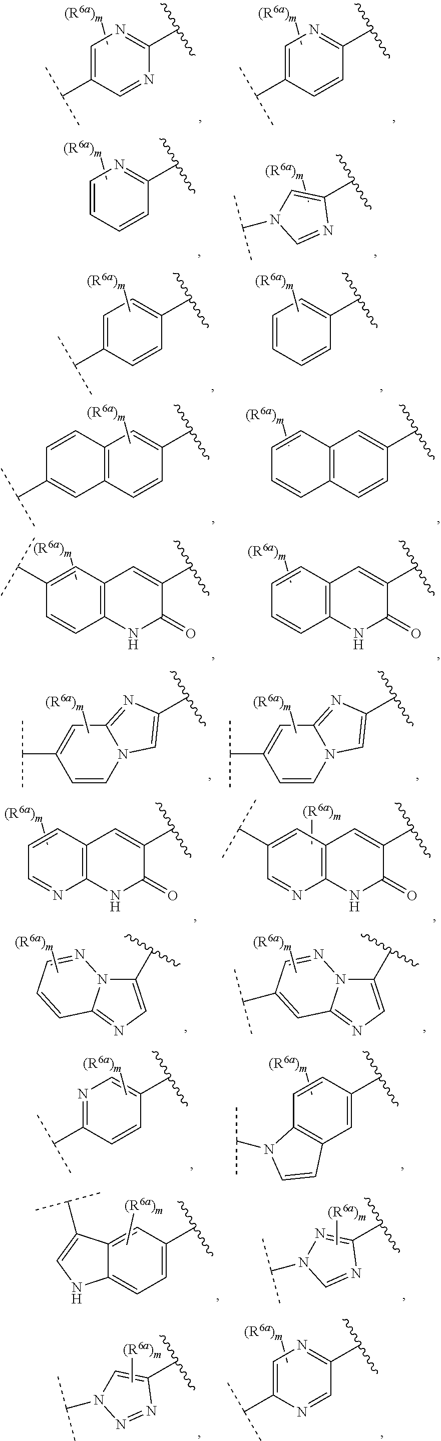

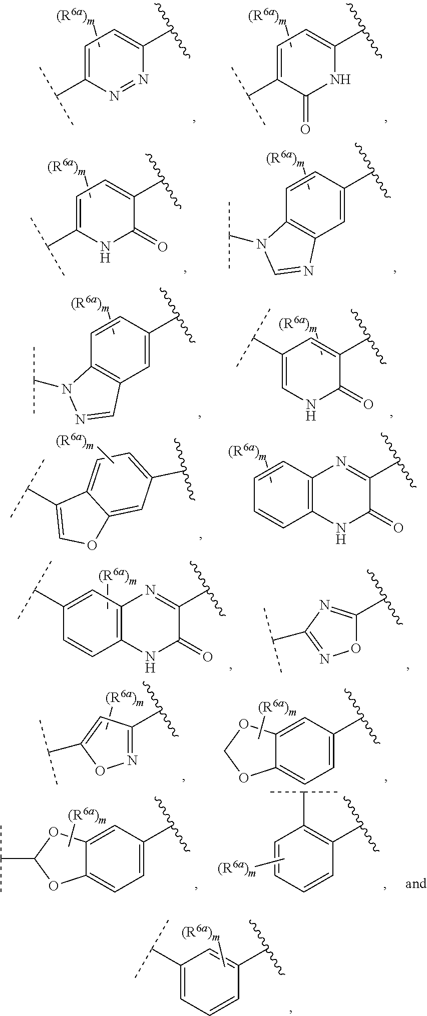

wherein wavy line indicates attachment points to the alkylamine and dotted line indicates attachment points to the L; and R^{6a} and m are as defined for formula (IA). When A ring is bicyclic, any one ring or both rings may be substituted by the same or different R^{6a}.

In some embodiments of a compound of formula (IA), A optionally substituted with R^{6a} is selected from the group consisting of

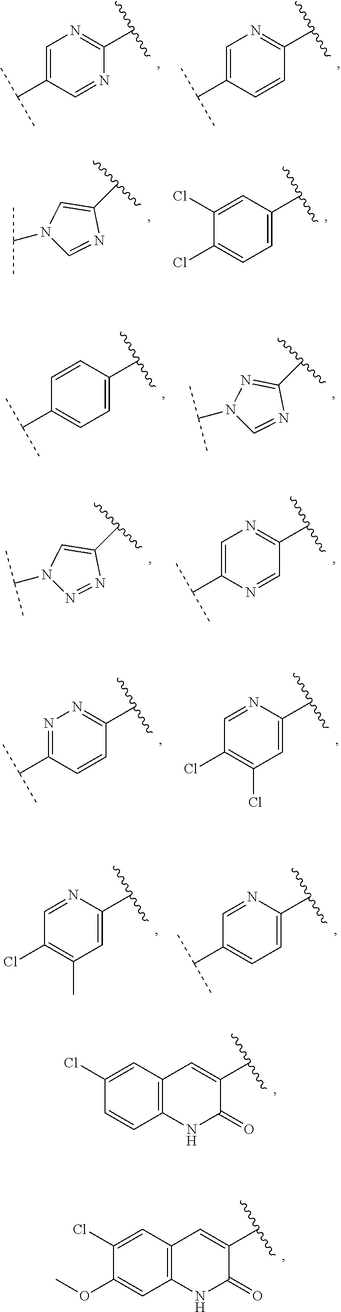

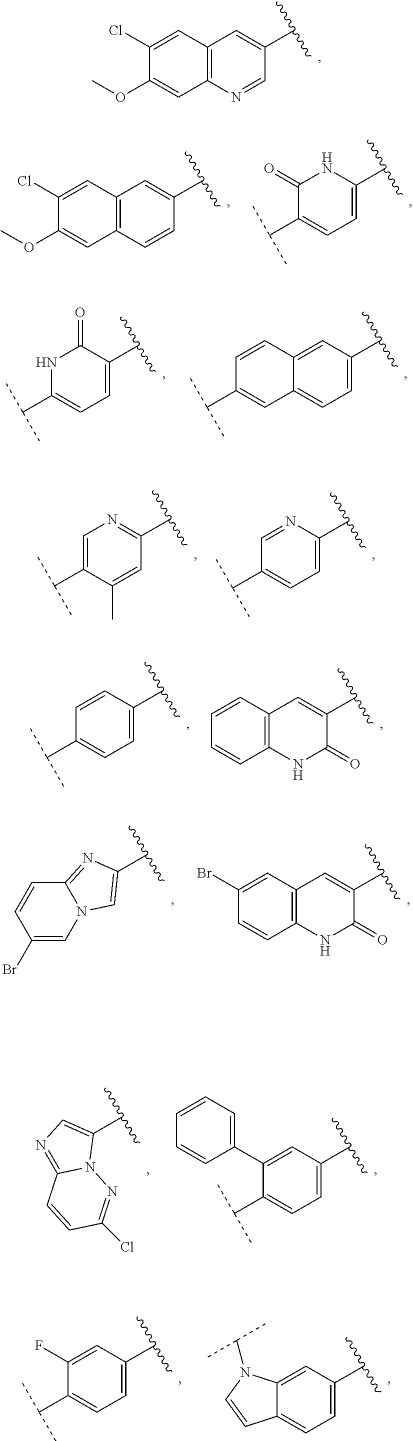

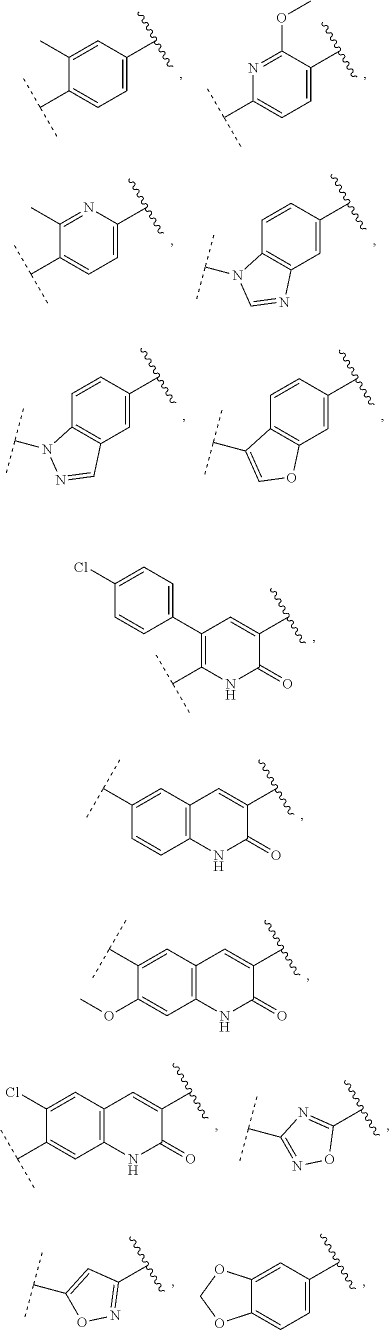

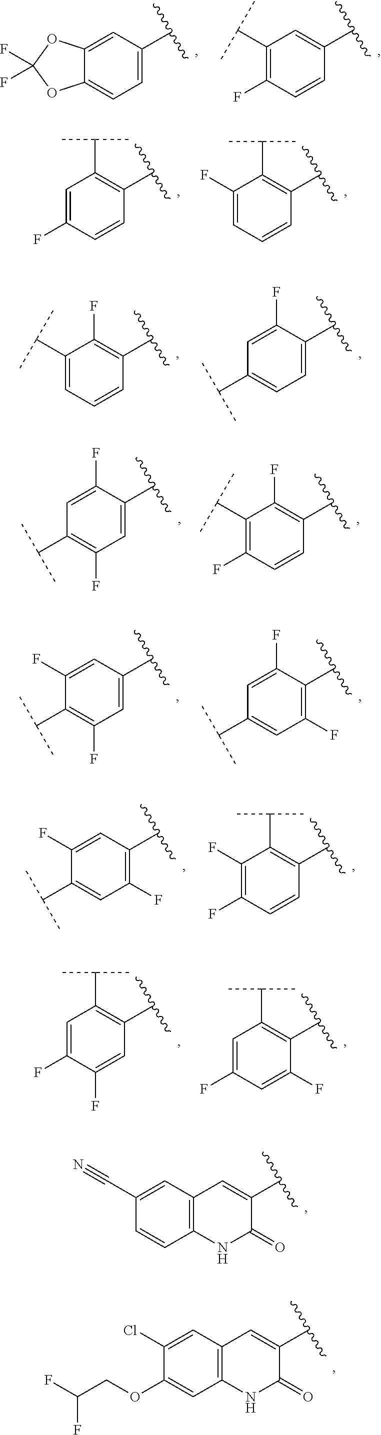

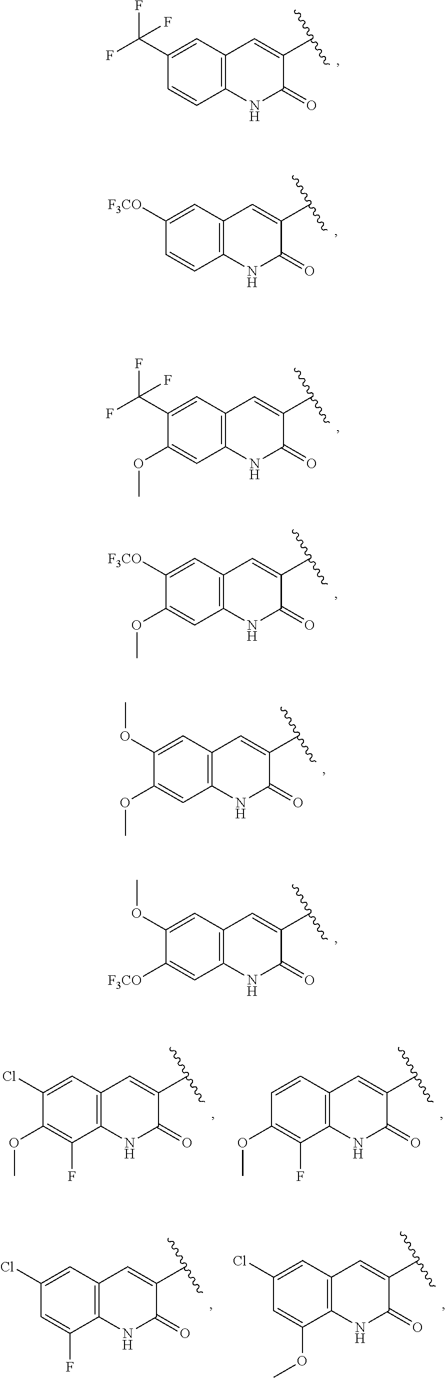

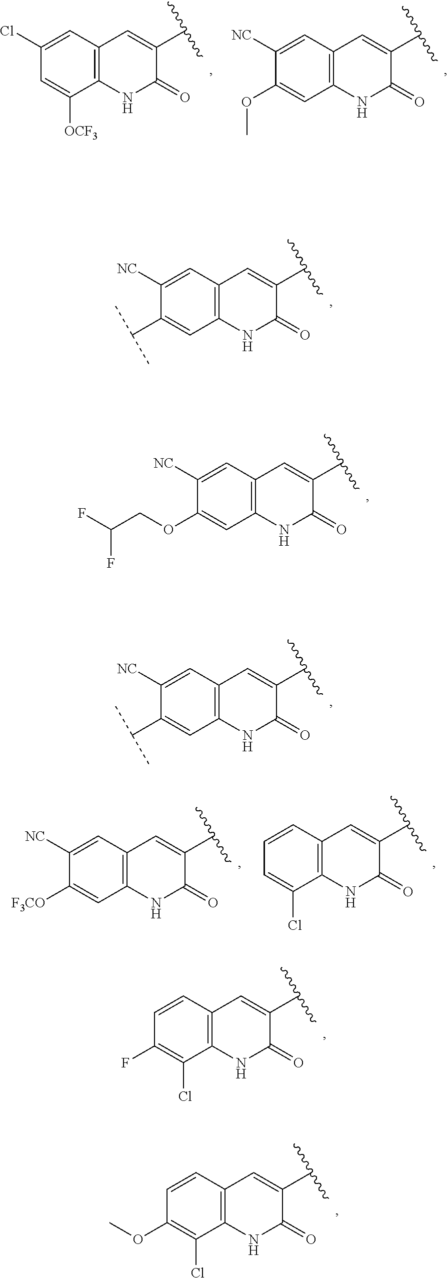

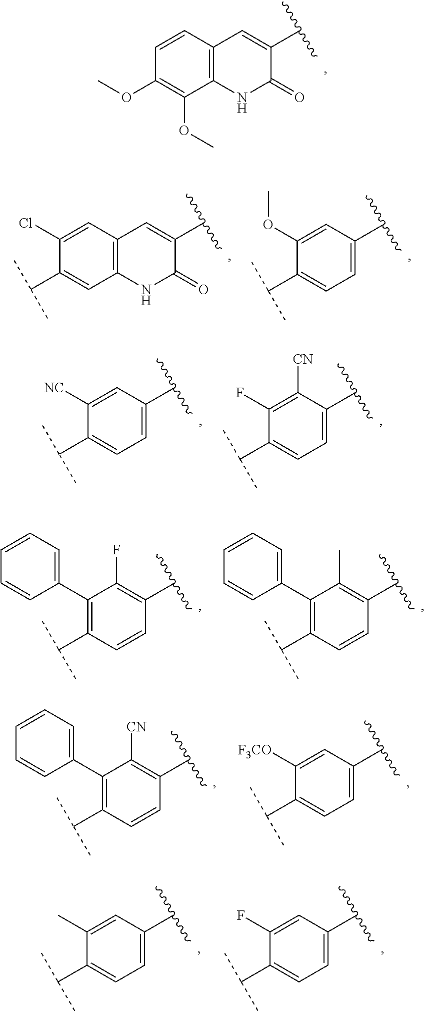

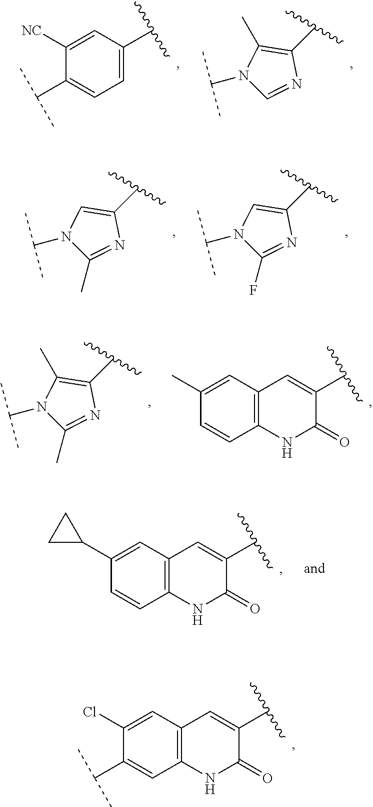

wherein wavy line indicates attachment points to the alkylamine and dotted line indicates attachment points to the L.

In some embodiments of a compound of formula (IA), B is hydrogen, C₆-C₁₀ aryl, 5- to 10-membered heteroaryl, C₃-C₈ cycloalkyl or 3- to 10-membered heterocyclyl, wherein each of which is optionally substituted by R^{6b}. In some embodiments of a compound of formula (IA), B is hydrogen. In some embodiments of a compound of formula (IA), B is C₆-C₁₀ aryl optionally substituted by R^{6b}. In some embodiments of a compound of formula (IA), B is phenyl optionally substituted by R^{6b}. In some embodiments of a compound of formula (IA), B is phenyl optionally substituted by halogen. In some embodiments of a compound of formula (IA), B is unsubstituted phenyl. In some embodiments of a compound of formula (IA), B is phenyl substituted by —F, —Cl, —Br, —CN, —OCH₃, —OCF₃, —CH₃ or —CF₃.

In some embodiments of a compound of formula (IA), B is naphthyl optionally substituted by R^{6b}.

In some embodiments of a compound of formula (IA), B is 5- to 10-membered heteroaryl optionally substituted by R^{6b}. In some embodiments of a compound of formula (IA), B is 5-membered heteroaryl optionally substituted by R^{6b}. In some embodiments of a compound of formula (IA), B is 6-membered heteroaryl optionally substituted by R^{6b}. In some embodiments of a compound of formula (IA), B is 9-membered bicyclic heteroaryl optionally substituted by R^{6b}, in which any one ring or both rings may be substituted by the same or different R^{6b}. In some embodiments of a compound of formula (IA), B is 10-membered bicyclic heteroaryl optionally substituted by R^{6b}, in which any one ring or both rings may be substituted by the same or different R^{6b}.

In some embodiments of a compound of formula (IA), B is 5-membered heteroaryl selected from imidazolyl, pyrrolyl, pyrazolyl, triazole, thiophenyl, furanyl, thiazolyl, isothiazolyl, 1,3,4-thiadiazolyl, oxazolyl, isoxazolyl and 1,3,4-oxadiazolyl, wherein each of which is optionally substituted by R^{6b}, wherein R^{6b} is selected from C₁-C₆ alkyl, —CN, halogen, C₁-C₆ alkoxy, C₁-C₆ haloalkoxy or C₁-C₆ haloalkyl. In some embodiments of a compound of formula (IA), B is triazolyl substituted by —CH₃.

In some embodiments of a compound of formula (IA), B is 6-membered heteroaryl selected from pyridyl, pyrimidyl, pyridazinyl or pyrazinyl, wherein each of which is optionally substituted by R^{6b}. wherein R^{6b} is selected from oxo, C₁-C₆ alkyl, —CN, halogen, C₁-C₆ alkoxy, C₁-C₆ haloalkoxy or C₁-C₆ haloalkyl. In some embodiments, R^{6b} is —CH₃, —CF₃, F or —Cl. In some embodiments of a compound of formula (IA), B is pyridyl, pyrimidyl, pyridazinyl or pyrazinyl optionally substituted by cyclopropyl, —F, —Cl, —Br, —CN, —OCH₃, —OCF₃, —CH₃ or —CF₃.

In some embodiments of a compound of formula (IA), B is 9-membered heteroaryl selected from indolyl, indazolyl, benzoimidazolyl, pyrrolopyridinyl pyrrolopyridazinyl, pyrrolopyrimidinyl, pyrazolopyridinyl, pyrazolopyrimidinyl, imidazopyridinyl, purinyl, benzofuranyl, furopyridinyl, benzooxazolyl, benzothiophenyl, benzothiazolyl, oxazolopyridinyl, thiazolopyridinyl or thienopyridinyl, wherein each of which is optionally substituted by R^{6b}.

In some embodiments of a compound of formula (IA), B is 10-membered heteroaryl selected from quinolinyl, quinolonyl, naphthyridinyl, quinazolinyl, pyridopyrimidinyl, cinnolinyl or pyridopyridazinyl, wherein each of which is optionally substituted by R^{6b}.

In some embodiments of a compound of formula (IA), B is 3- to 10-membered heterocyclyl optionally substituted by R^{6b}. In some embodiments of a compound of formula (IA), 3- to 10-membered heterocyclyl of B is selected from aziridinyl, azetidinyl, pyrrolidinyl, piperidinyl, piperazinyl, morpholinyl, thiomorpholinyl, pyrrolidinyl, oxetanyl, tetrahydrofuranyl or azepanyl, wherein each of which is optionally substituted by R^{6b} wherein R^{6b} is selected from oxo, C₁-C₆ alkyl, —CN, halogen, C₁-C₆ alkoxy, C₁-C₆ haloalkoxy, —C(O)R¹³ or C₁-C₆ haloalkyl. In some embodiments, R^{6b} is oxo, —C(O)CH₃, —C(O)CH═CH₂ or —CH₃. In some embodiments, heterocyclyl ring may be fused with aryl or heteraryl ring to form biycylic system which is optionally substituted R^{6b}, in which one ring or both rings may be substituted by the same or different R^{6b}. In some embodiments, heterocyclyl ring may be saturated or partially unsaturated.

In some embodiments of a compound of formula (IA), B is C₃-C₈ cycloalkyl optionally substituted by R^{6b}. In some embodiments of a compound of formula (IA), B is cylopropyl, cyclobutyl or cyclopentyl optionally substituted by —CN or —CH₃.

In some embodiments of a compound of formula (IA), B is selected from the group consisting of:

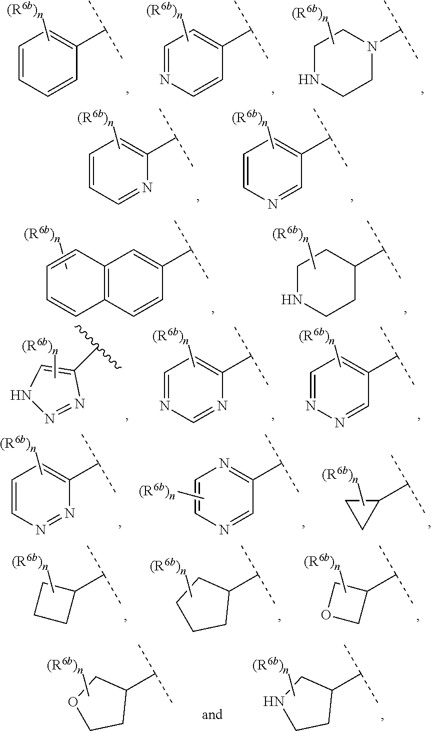

wherein dotted line indicates attachment points to the L; and R^{6b} and n are as defined for formula (IA). When B ring is bicyclic, any one ring or both rings may be substituted by the same or different R^{6b}.

In some embodiments of a compound of formula (IA), B, substituted with R^{6b} is selected from the group consisting of:

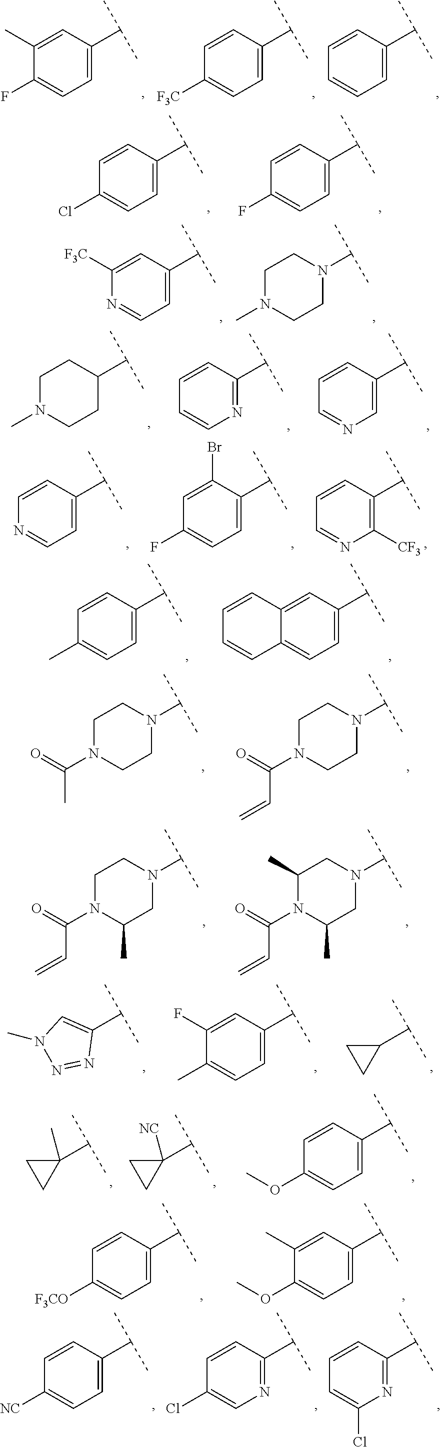

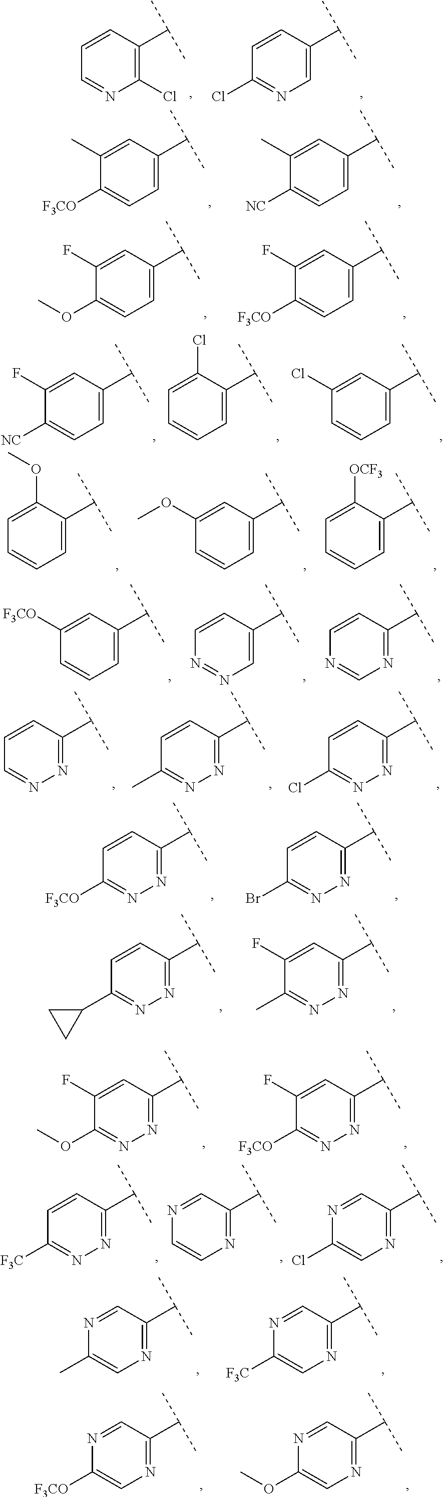

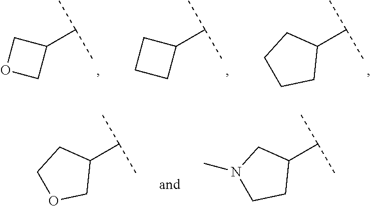

wherein the dotted lines denote attachment points to L.

In some embodiments of a compound of formula (IA), L is a bond. In some embodiments of a compound of formula (IA), L is —O—. In some embodiments of a compound of formula (IA), L is —(CH₂)₁₋₃—. In Some embodiments of a compound of formula (IA), L is —NH—. In some embodiments of a compound of formula (IA), L is —NCH₃—. In Some embodiments of a compound of formula (IA), L is —SO₂—. In some embodiments of a compound of formula (IA), L is —C(O)—. In some embodiments of a compound of formula (IA), L is —C(O)NH—. In some embodiments of a compound of formula (IA), L is —NHC(O)—. In some embodiments of a compound of formula (IA), L is —CH₂—O—. In some embodiments of a compound of formula (IA), L is —S—. In some embodiments of a compound of formula (IA), L is —CR^{b}R^{c}—, wherein R^{b} and R^{c} is independently hydrogen, halogen, —CN, C₁-C₆ alkyl, C₃-C₆ cycloalkyl, or —(C₁-C₃ alkylene)(C₃-C₆ cycloalkyl).

In some embodiments of a compound of formula (IA), m is 0. In some embodiments of a compound of formula (IA), m is 1. In some embodiments of a compound of formula (IA), m is 2. In some embodiments, m is 3. In some embodiments of a compound of formula (IA), m is or 4.

In some embodiments of a compound of formula (IA), n is 0. In some embodiments of a compound of formula (IA), n is 1. In some embodiments of a compound of formula (IA), n is 2. In some embodiments of a compound of formula (IA), n is 3. In some embodiments of a compound of formula (IA), n is or 4.

In some embodiments of a compound of formula (IA), R¹ is hydrogen. In some embodiments of a compound of formula (IA), R¹ is halogen. In some embodiments of a compound of formula (IA), R¹ is C₁-C₆ alkyl. In some embodiments of a compound of formula (IA), R¹ is —CH₃.

In some embodiments of a compound of formula (IA), R² is hydrogen, halogen, C₁-C₆ alkoxy, C₁-C₆ haloalkyl, C₁-C₆ haloalkoxy, —C(O)OR^{2a}, C₃-C₆ cycloalkyl, 3- to 6-membered heterocyclyl, C₆ aryl, 5- to 6-membered heteroaryl —(C₁-C₃ alkylene)C₆ aryl or C₁-C₆ alkyl optionally substituted by oxo, halogen, —OR^{2a} or —NR^{2a}R^{2b}, wherein C₃-C₆ cycloalkyl, 3- to 6-membered heterocyclyl, C₆ aryl, 5- to 6-membered heteroaryl and —(C₁-C₃ alkylene)C₆ aryl of R² optionally substituted by C₁-C₆ alkyl; In some embodiments of a compound of formula (IA), R² is hydrogen. In some embodiments of a compound of formula (IA), R² is halogen. In some embodiments of a compound of formula (IA), R² is C₁-C₆ alkoxy. In some embodiments of a compound of formula (IA), R² is —OCH₃. In some embodiments of a compound of formula (IA), R² is C₆ aryl or more specifically phenyl. In some embodiments of a compound of formula (IA), R² is 5- to 6-membered heteroaryl. In some embodiments of a compound of formula (IA), R² is 5-membered heteroaryl. In some embodiments of a compound of formula (IA), R² is 5-membered heteroaryl substituted with C₁-C₆ alkyl. In some embodiments of a compound of formula (IA), R² is

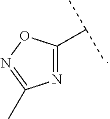

In some embodiments of a compound of formula (IA), R² is 6-membered heteroaryl or more specifically pyridyl. In some embodiments of a compound of formula (IA), R² is

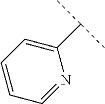

wherein dotted line indicates point of attachment. In some embodiments of a compound of formula (IA), R² is

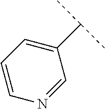

wherein dotted line indicates point of attachment. In some embodiments of a compound of formula (IA), R² is

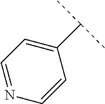

wherein dotted line indicates point of attachment. In some embodiments of a compound of formula (IA), R² is C₁-C₆ alkyl optionally substituted by oxo, halogen, —OR^{2a} or —NR^{2a}R^{2b}. In some embodiments of a compound of formula (IA), R² is unsubstituted C₁-C₆ alkyl. In some embodiments of a compound of formula (IA), R² is methyl. In some embodiments of a compound of formula (IA), R² is ethyl. In some embodiments of a compound of formula (IA), R² is isopropyl. In some embodiments of a compound of formula (IA), R² is isobutyl. In some embodiments of a compound of formula (IA), R² is C₁-C₆ alkyl substituted by —OR^{2a}, wherein R^{2a} is hydrogen or C₁-C₆ alkyl. In some embodiments of a compound of formula (IA), R² is C₁-C₆ alkyl substituted by —OH. In some embodiments of a compound of formula (IA), R² is C₁-C₆ alkyl substituted by —OCH₃. In some embodiments of a compound formula (IA), R² is C₁-C₆ alkyl substituted by —OCH₂CH₃. In some embodiments of a compound formula (IA), R² is C₁-C₆ alkyl substituted by —OCH(CH₃)₂. In some embodiments of a compound of formula (IA), R² is C₁-C₆ alkyl substituted by —OC(CH₃)₃. In some embodiments of a compound of formula (IA), R² is —CH(OH)CH₃. In some embodiments of a compound of formula (IA), R² is C₁-C₆ alkyl substituted by —NR^{2a}R^{2b}. In some embodiments of a compound of formula (IA), R² is C₁-C₆ alkyl substituted by —NH₂. In some embodiments of a compound of formula (IA), R² is —CH(CH₃)NH₂. In some embodiments of a compound of formula (IA), R² is C₁-C₆ alkyl substituted by NHCH₃. In some embodiments of a compound of formula (IA), R² is —CH(CH₃)NHCH₃. In some embodiments of a compound of formula (IA), R² is C₁-C₆ alkyl substituted by —N(CH₃)₂. In some embodiments of a compound of formula (IA), R² is —CH(CH₃)—N(CH₃)₂. In some embodiments of a compound of formula (IA), R² is C₁-C₆ alkyl substituted by halogen. In some embodiments of a compound of formula (IA), R² is C₁-C₆ alkyl substituted by one or more —F. In some embodiments of a compound of formula (IA), R² is C₁-C₆ alkyl substituted by monofluoro. In some embodiments of a compound of formula (IA), R² is —CH(CH₃)CH₂F. In some embodiments of a compound of formula (IA), R² is —CH₂F. In some embodiments of a compound of formula (IA), R² is —C₂H₅F. In some embodiments of a compound of formula (IA), R² is C₁-C₆ alkyl substituted by difluoro. In some embodiments of a compound of formula (IA), R² is —CHF₂. In some embodiments of a compound of formula (IA), R² is —CH(CH₃)CHF₂. In some embodiments of a compound of formula (IA), R² is —CH(CH₂F)₂. In some embodiments of a compound of formula (IA), R² is C₁-C₆ alkyl substituted by trifluoro. In some embodiments of a compound of formula (IA), R² is —CF₃. In some embodiments of a compound of formula (IA), R² is —CH(CH₃)CF₃. In some embodiments of a compound of formula (IA), R² is C₃-C₆ cycloalkyl. In some embodiments of a compound of formula (IA), R² is cyclopropyl. In some embodiments of a compound of formula (IA), R² is —(C₁-C₃ alkylene)C₆ aryl. In some embodiments of a compound of formula (IA), R² is —(CH₂)phenyl. In some embodiments of a compound of formula (IA), R¹ and R² are taken together with the atom to which they are attached to form a C₃-C₆ cycloalkyl or 3- to 6-membered heterocyclyl, each of which is optionally substituted by oxo, —OH, halogen, —NH₂, or C₁-C₆ alkyl optionally substituted by oxo, —OH, halogen or —NH₂. In some embodiments of a compound of formula (IA), R¹ and R² are taken together with the atom to which they are attached to form cyclopropyl.

In some embodiments of a compound of formula (IA), R¹ and R² both are hydrogen. In some embodiments of a compound of formula (IA), R¹ and R² both are C₁-C₆ alkyl. In some embodiments of a compound of formula (IA), R¹ and R² both are methyl. In some embodiments of a compound of formula (IA), R¹ is hydrogen and R² is C₁-C₆ alkyl. In some embodiments of a compound of formula (IA), R¹ is hydrogen and R² is —CH₃. In some embodiments of a compound of formula (IA), R¹ is hydrogen and R² is ethyl. In some embodiments of a compound of formula (IA), R¹ is hydrogen and R² is propyl. In some embodiments of a compound of formula (IA), R¹ is hydrogen and R² is isopropyl. In some embodiments of a compound of formula (IA), R¹ is hydrogen and R² is isobutyl. In some embodiments of a compound of formula (IA), R¹ is hydrogen and R² is —CH₂F. In some embodiments of a compound of formula (IA), R¹ is hydrogen and R² is —CHF₂. In some embodiments of a compound of formula (IA), R¹ is hydrogen and R² is —CH(CH₃)CHF₂. In some embodiments of a compound of formula (IA), R¹ is hydrogen and R² is phenyl. In some embodiments of a compound of formula (IA), R¹ is hydrogen and R² is cyclopropyl. In some embodiments of a compound of formula (IA), R¹ is hydrogen and R² is —(C₁-C₃ alkylene)C₆ aryl. In some embodiments of a compound of formula (IA), R¹ is hydrogen and R² is —(CH₂)phenyl. In some embodiments of a compound of formula (IA), R¹ is hydrogen and R² is 5-membered heteroaryl. In some embodiments of a compound of formula (IA), R¹ is hydrogen and R² is 5-membered heteroaryl substituted with C₁-C₆ alkyl. In some embodiments of a compound of formula (IA), R¹ is hydrogen and R² is

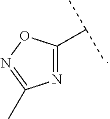

In some embodiments of a compound of formula (IA), R¹ is hydrogen and R² is 6-membered heteroaryl or more specifically pyridyl. In some embodiments of a compound of formula (IA), R¹ is hydrogen and R² is

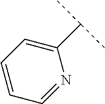

wherein dotted line indicates point of attachment. In some embodiments of a compound of formula (IA), R¹ is hydrogen and R² is

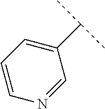

wherein dotted line indicates point of attachment. In some embodiments of a compound of formula (IA), R¹ is hydrogen and R² is

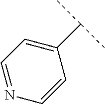

wherein dotted line indicates point of attachment.

In some embodiments of a compound of formula (IA), R³ is hydrogen, halogen, or C₁-C₆ alkyl optionally substituted by oxo, —OH or halogen. In some embodiments of a compound of formula (IA), R³ is hydrogen. In some embodiments of a compound of formula (IA), R³ is halogen. In some embodiments of a compound of formula (IA), R³ is C₁-C₆ alkyl optionally substituted by oxo, —OH or halogen.

In some embodiments of a compound of formula (IA), R⁴ is hydrogen, halogen, or C₁-C₆ alkyl optionally substituted by oxo, —OH or halogen. In some embodiments of a compound of formula (IA), R⁴ is hydrogen. In some embodiments of a compound of formula (IA), R⁴ is halogen. In some embodiments of a compound of formula (IA), R⁴ is C₁-C₆ alkyl optionally substituted by oxo, —OH or halogen.

In some embodiments of a compound of formula (IA), R³ is hydrogen and R⁴ is halogen, or C₁-C₆ alkyl optionally substituted by oxo, —OH or halogen. In some embodiments of a compound of formula (IA), R³ is hydrogen and R⁴ is halogen. In some embodiments of a compound of formula (IA), R³ is hydrogen and R⁴ is C₁-C₆ alkyl optionally substituted by oxo, —OH or halogen. In some embodiments of a compound of formula (IA), R³ is hydrogen and R⁴ is —CH₃. In some embodiments of a compound of formula (IA), R⁴ is hydrogen and R³ is halogen, or C₁-C₆ alkyl optionally substituted by oxo, —OH or halogen. In some embodiments of a compound of formula (IA), R⁴ is hydrogen and R³ is halogen. In some embodiments of a compound of formula (IA), R⁴ is hydrogen and R³ is C₁-C₆ alkyl optionally substituted by oxo, —OH or halogen. In some embodiments of a compound of formula (IA), R⁴ is hydrogen and R³ is —CH₃. In some embodiments of a compound of formula (IA), R³ and R⁴ both are hydrogen. In some embodiments of a compound of formula (IA), R³ and R⁴ both are —CH₃.

In some embodiments of a compound of formula (IA), R³ and R⁴ are taken together with the atom to which they are attached to form a C₃-C₆ cycloalkyl or 3-6 membered heterocyclyl, each of which is optionally substituted by oxo, —OH, halogen, —NH₂, or C₁-C₆ alkyl optionally substituted by oxo, —OH, halogen or —NH₂. In some embodiments of a compound of formula (IA), R³ and R⁴ are taken together with the atom to which they are attached to form cyclopropyl. In some embodiments of a compound of formula (IA), R³ and R⁴ are taken together with the atom to which they are attached to form oxytanyl ring.

In some embodiments of a compound of formula (IA), R⁵ is hydrogen, C₁-C₆ alkyl, C₂-C₆ alkenyl, C₂-C₆ alkynyl, C₃-C₆ cycloalkyl, 3- to 6-membered heterocyclyl, C₆-aryl, 5- to 6-membered heteroaryl, —CN, halogen, C₁-C₆ alkoxy, C₁-C₆ haloalkoxy, —OR¹⁰, —SR¹⁰, —S(O)₂R¹⁰, —S(O)₂NR¹¹R¹², —NR¹⁰S(O)₂R¹¹, —NR¹¹R¹², —C(O)R¹⁰, —NR¹⁰C(O)R¹¹, —NR¹⁰C(O)NR¹¹R¹², —C(O)OR¹⁰, —C(O)ONR¹¹R¹², —C(O)NR¹¹R¹², wherein each of which is optionally substituted by R⁸. In some embodiments of a compound of formula (IA), R⁵ is hydrogen. In some embodiments of a compound of formula (IA), R⁵ is C₁-C₆ alkyl optionally substituted by R⁸. In some embodiments of a compound of formula (IA), R⁵ is C₃-C₆ cycloalkyl optionally substituted by R⁸. In some embodiments of a compound of formula (IA), R⁵ is 3- to 6-membered heterocyclyl optionally substituted by R⁸. In some embodiments of a compound of formula (IA), R⁵ is —CN. In some embodiments of a compound of formula (IA), R⁵ is halogen. In some embodiments of a compound of formula (IA), R⁵ is C₁-C₆ alkoxy. In some embodiments of a compound of formula (IA), R⁵ is C₁—C₆ haloalkoxy. In some embodiments of a compound of formula (IA), R⁵ is —OR¹⁰. In some embodiments of a compound of formula (IA), R⁵ is —S(O)₂R¹⁰. In some embodiments of a compound of formula (IA), R⁵ is —OR¹⁰. In some embodiments of a compound of formula (IA), R⁵ is —S(O)₂NR¹¹R¹². In some embodiments of a compound of formula (IA), R⁵ is —NR¹⁰S(O)₂R¹¹. In some embodiments of a compound of formula (IA), R⁵ is —C(O)R¹⁰. In some embodiments of a compound of formula (IA), R⁵ is —NR¹⁰C(O)R¹¹. In some embodiments of a compound of formula (IA), R⁵ is —C(O)OR¹⁰. In some embodiments of a compound of formula (IA), R⁵ is —C(O)NR¹¹R¹².

In some embodiments of a compound of formula (IA), R⁵ is hydrogen. In some embodiments of a compound of formula (IA), R⁵ is methyl. In some embodiments of a compound of formula (IA), R⁵ is ethyl. In some embodiments of a compound of formula (IA), R⁵ is ter-butyl. In some embodiments of a compound of formula (IA), R⁵ is iso-butyl. In some embodiments of a compound of formula (IA), R⁵ is cyclopropyl. In some embodiments of a compound of formula (IA), R⁵ is phenyl. In some embodiments of a compound of formula (IA), R⁵ is —CN. In some embodiments of a compound of formula (IA), R⁵ is —N(CH₃)₂. In some embodiments of a compound of formula (IA), R⁵ is —Cl. In some embodiments of a compound of formula (IA), R⁵ is —Br. In some embodiments of a compound of formula (IA), R⁵ is —OCH₃. In some embodiments of a compound of formula (IA), R⁵ is —OC₃H₇. In some embodiments of a compound of formula (IA), R⁵ is —OCH(CH₃)₂. In some embodiments of a compound of formula (IA), R⁵ is —OCF₃. In some embodiments of a compound of formula (IA), R⁵ is —CF₃. In some embodiments of a compound of formula (IA), R⁵ is —SCH₃. In some embodiments of a compound of formula (IA), R⁵ is aziridinyl. In some embodiments of a compound of formula (IA), R⁵ is piperidinyl. In some embodiments of a compound of formula (IA), R⁵ is propyne. In some embodiments of a compound of formula (IA), R⁵ is —SO₂NHCH₃. In some embodiments of a compound of formula (IA), R⁵ is C(O)OCH₃. In some embodiments of a compound of formula (IA), R⁵ is isopropene. In some embodiments of a compound of formula (IA), R⁵ is thiazolyl.

In some embodiments of a compound of formula (IA), R⁷ and R^{7′} are independently hydrogen, C₃-C₆ cycloalkyl or C₁-C₆ alkyl optionally substituted by halogen or —OH. In some embodiments of a compound of formula (IA), R⁷ and R^{7′} both are hydrogen. In some embodiments of a compound of formula (IA), R⁷ and R^{7′} both are CH₃. In some embodiments of a compound of formula (IA), R⁷ is hydrogen and R^{7′} is —CH₃. In some embodiments of a compound of formula (IA), R⁷ is hydrogen and R^{7′} is ethyl. In some embodiments of a compound of formula (IA), R⁷ is hydrogen and R^{7′} is isopropyl. In some embodiments of a compound of formula (IA), R⁷ is hydrogen and R^{7′} is n-propyl. In some embodiments of a compound of formula (IA), R⁷ is hydrogen and R^{7′} is ter-butyl. In some embodiments of a compound of formula (IA), R⁷ is hydrogen and R^{7′} is cyclopropyl. In some embodiments of a compound of formula (IA), R⁷ is hydrogen and R^{7′} is cyclobutyl. In some embodiments of a compound of formula (IA), R⁷ is hydrogen and R^{7′} is —CF₃. In some embodiments of a compound of formula (IA), R⁷ is hydrogen and R^{7′} is —CH₂F. In some embodiments of a compound of formula (IA), R⁷ is methyl and R^{7′} is isopropyl.

In some embodiments of a compound of formula (IA), R⁷ and R^{7′} are taken together with the atom to which they are attached to form a C₃-C₆ cycloalkyl. In some embodiments of a compound of formula (IA), R⁷ and R^{7′} are taken together with the atom to which they are attached to form a cyclopropyl.

In some embodiments of a compound of formula (IA), A, B, L, R^{6a′} R^{6b}, m and n are together is

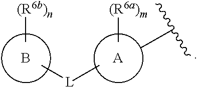

In some embodiments of a compound of formula (IA), A, B, L, R^{6a}, R^{6b}, m and n are together is

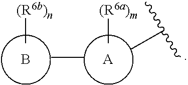

In some embodiments of a compound of formula (IA), A, B, L, R^{6a}, and m are together is

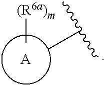

In some embodiments of a compound of formula (IA), A, B, L, R^{6a}, R^{6b}, m and n are together selected from the group of

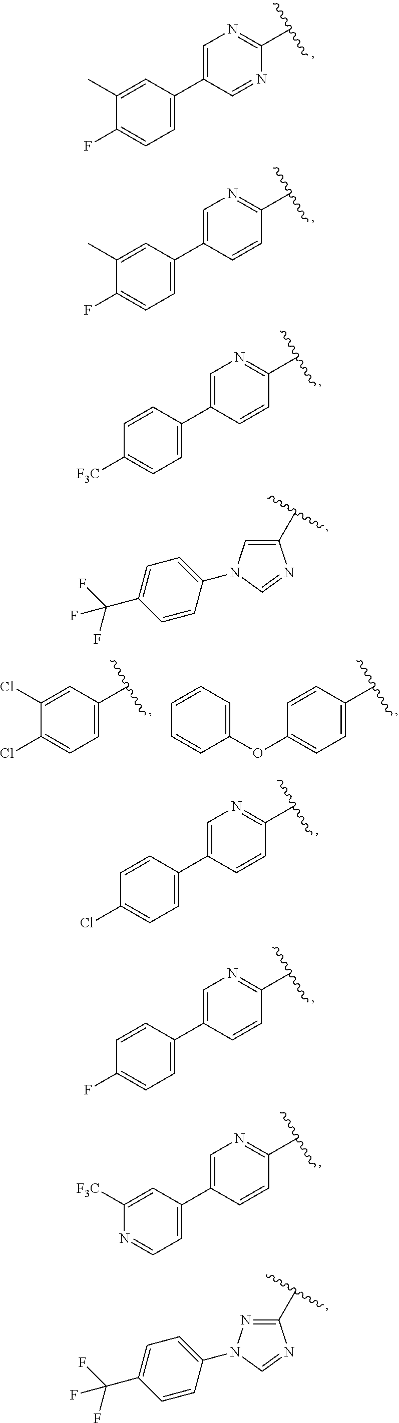

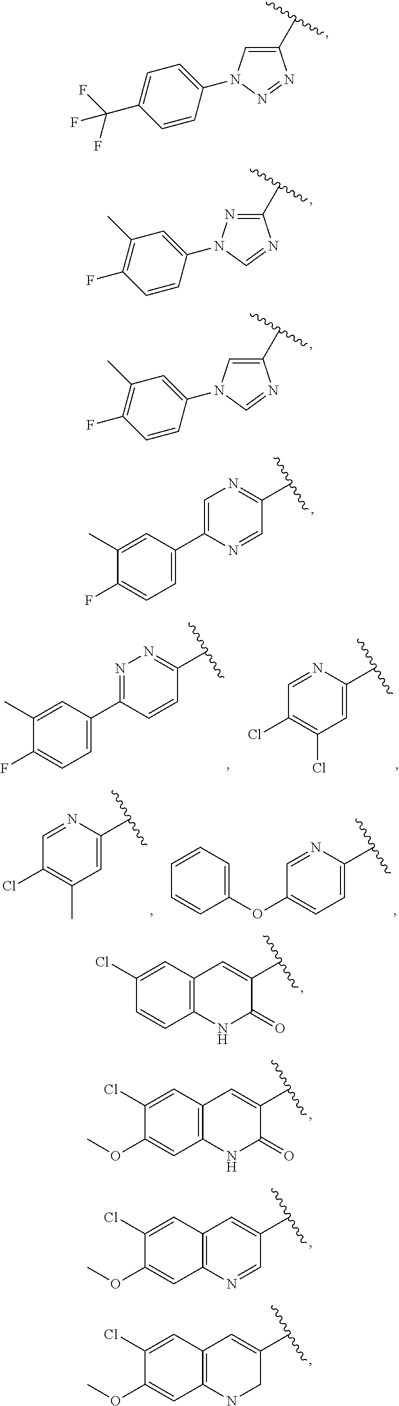

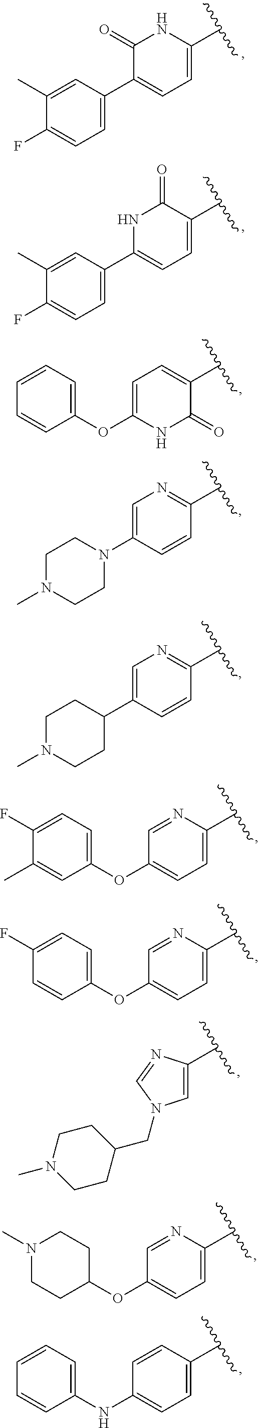

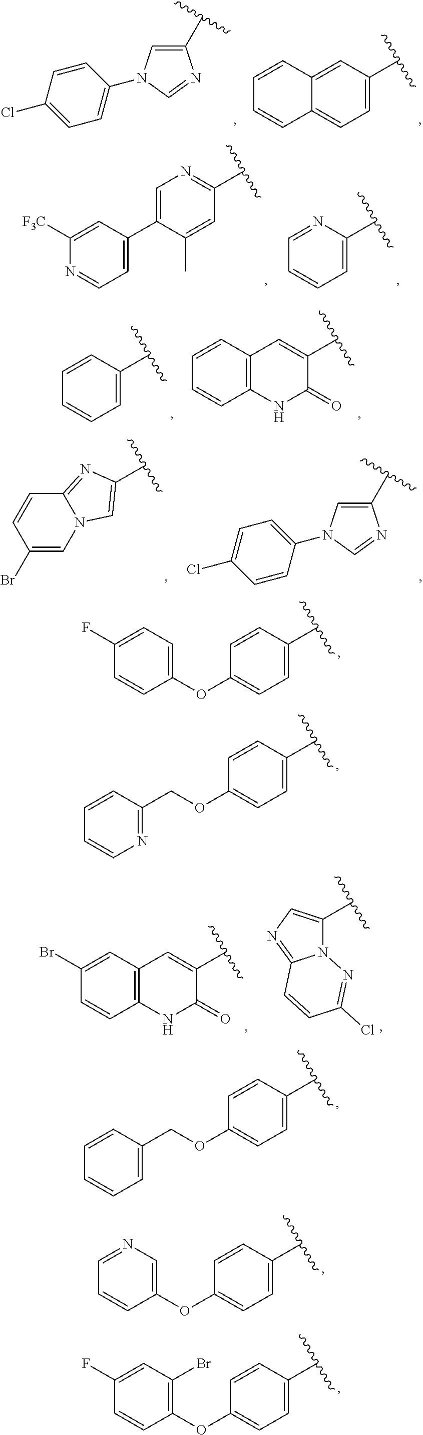

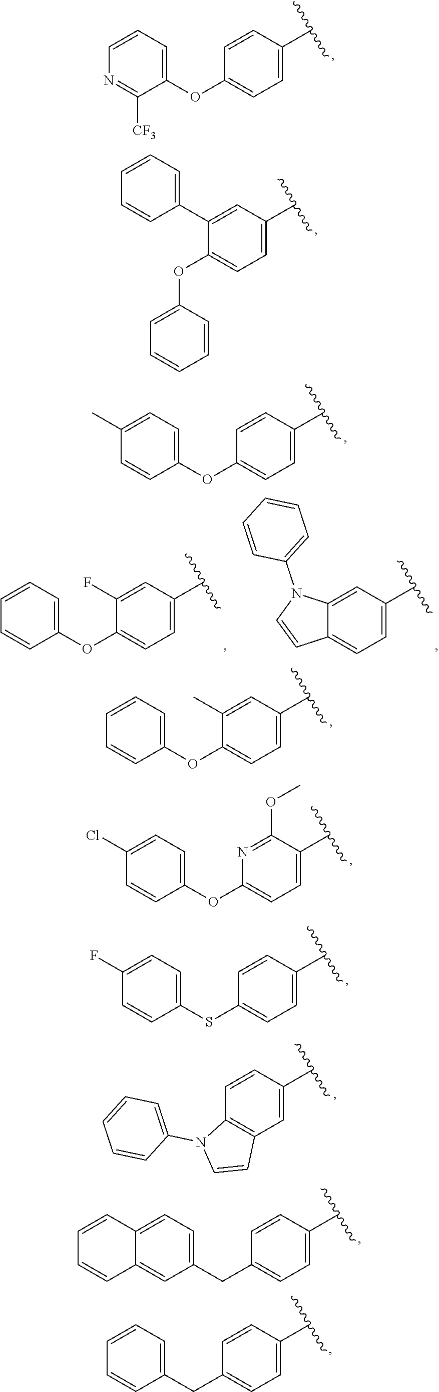

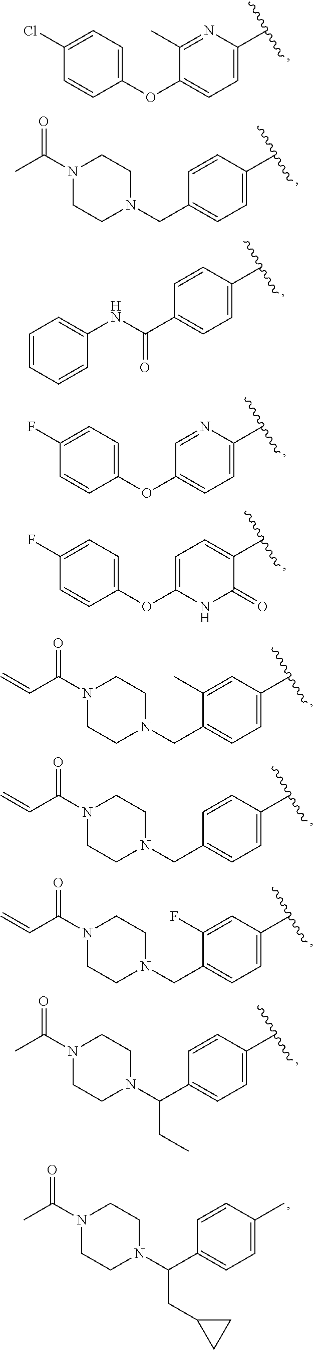

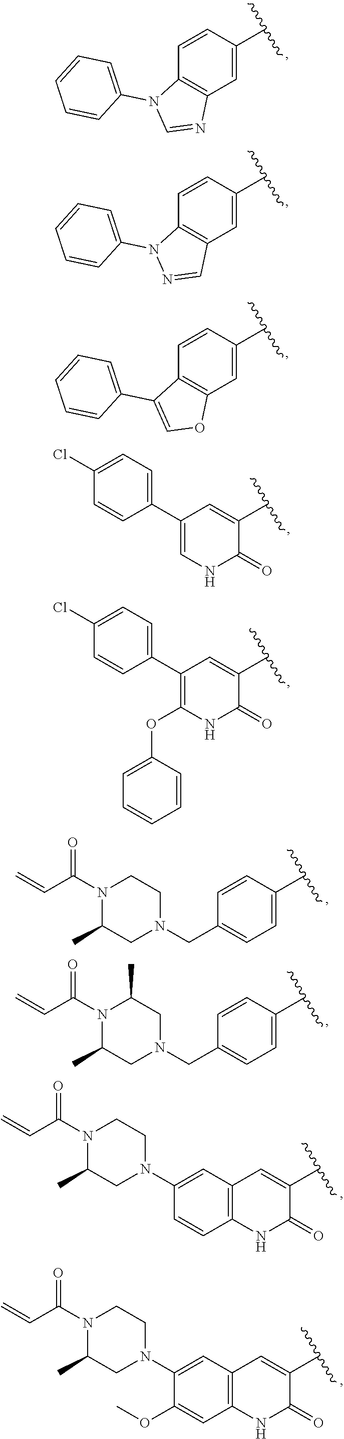

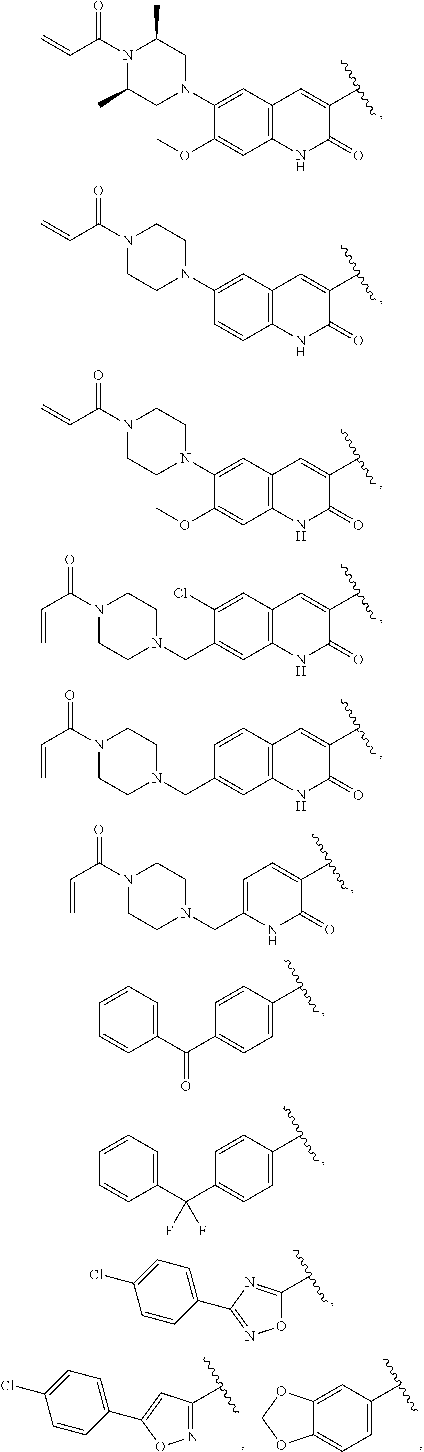

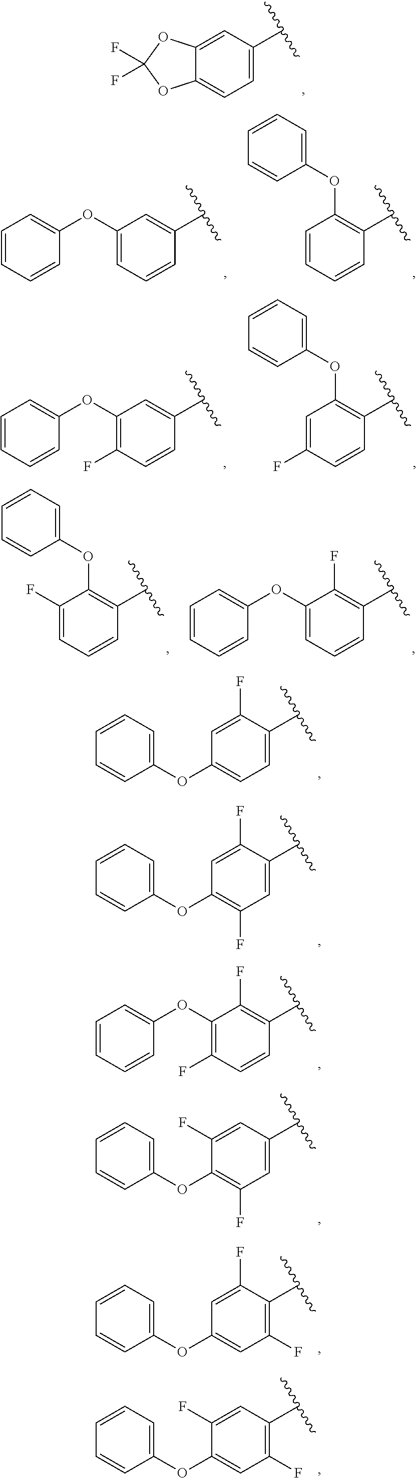

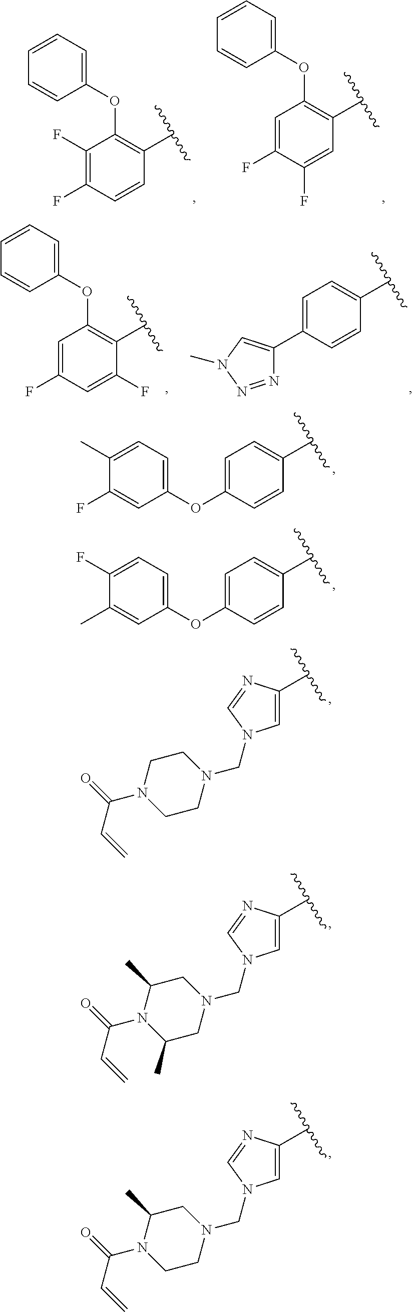

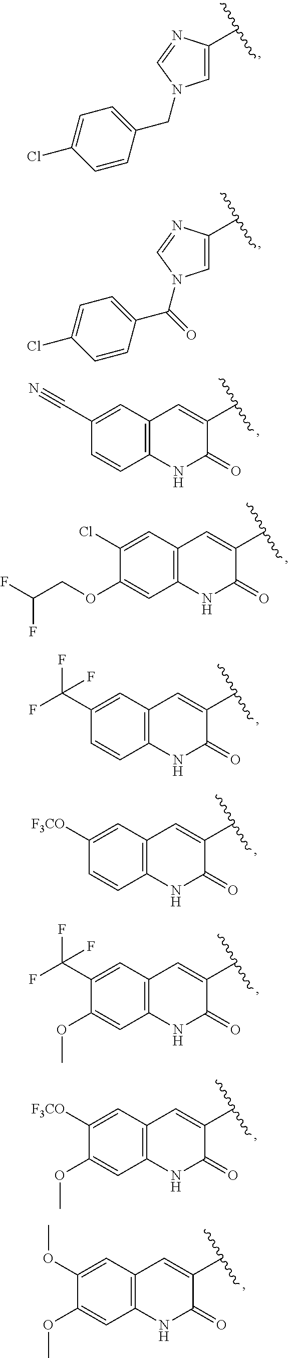

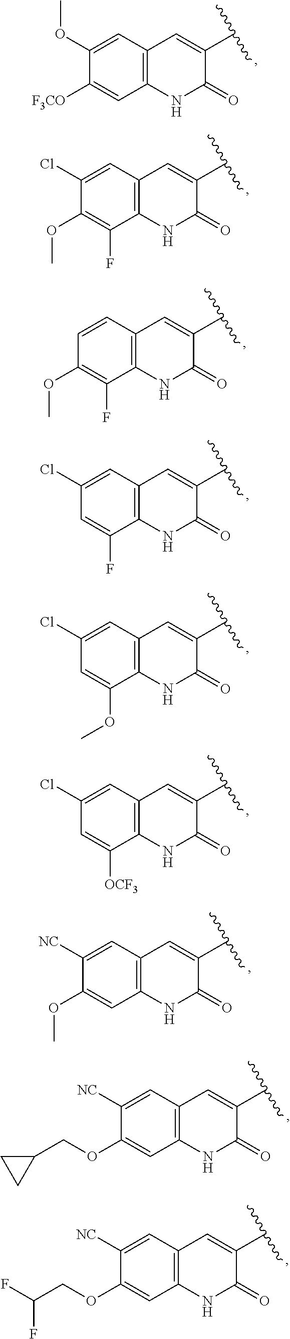

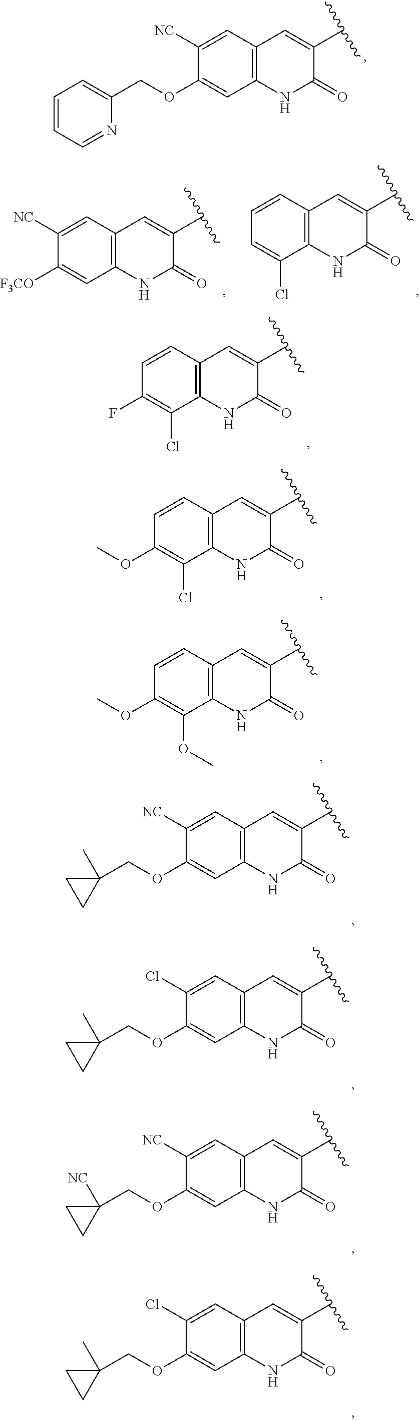

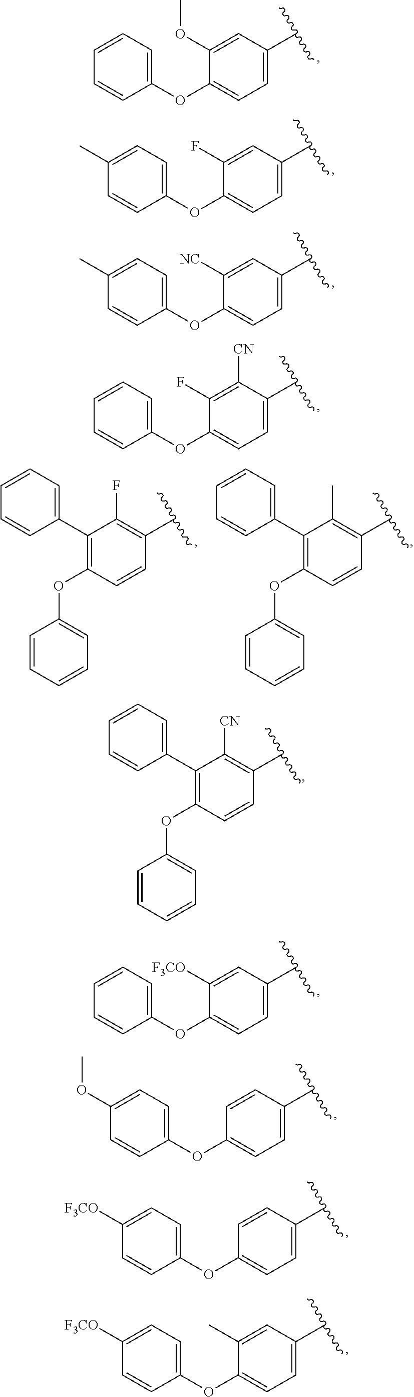

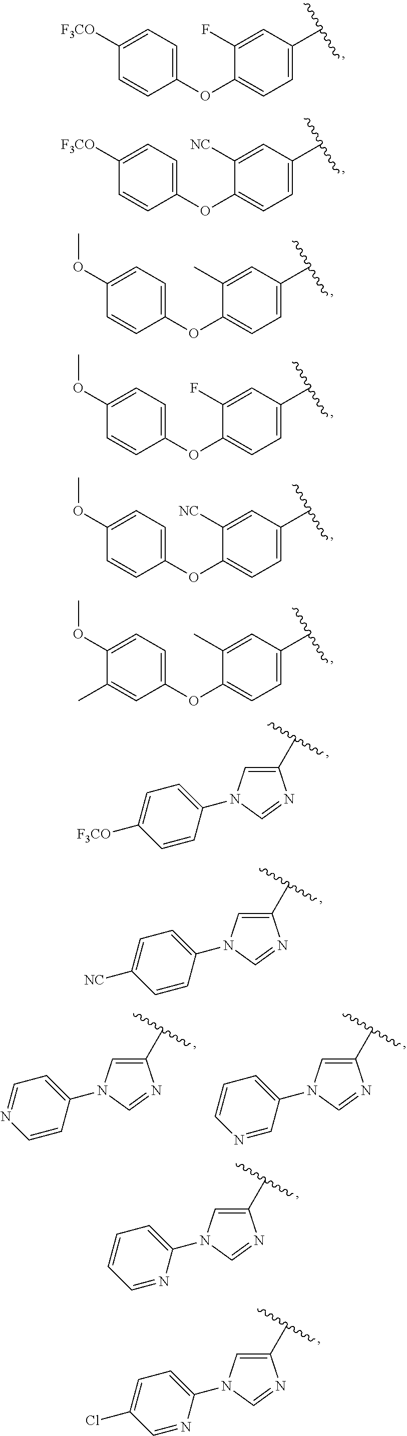

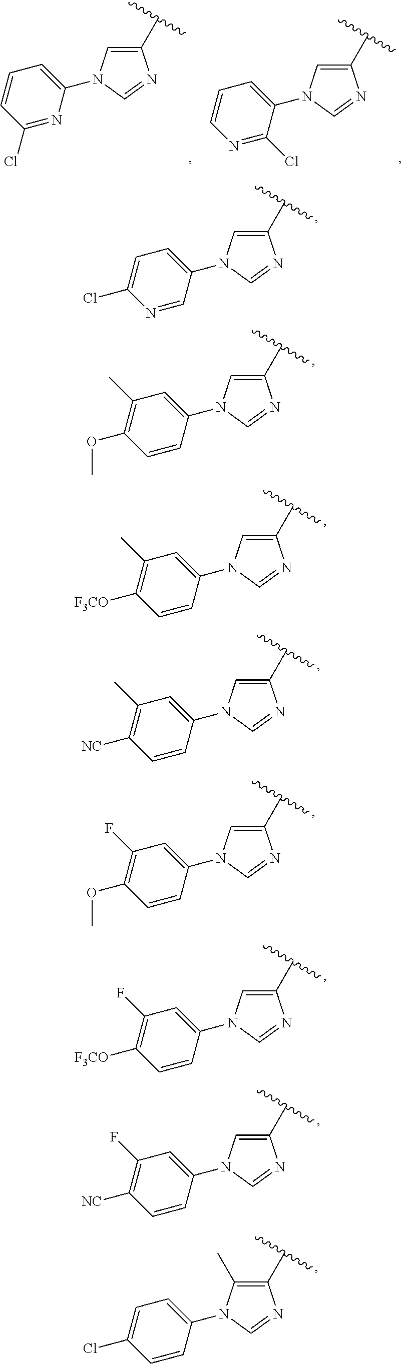

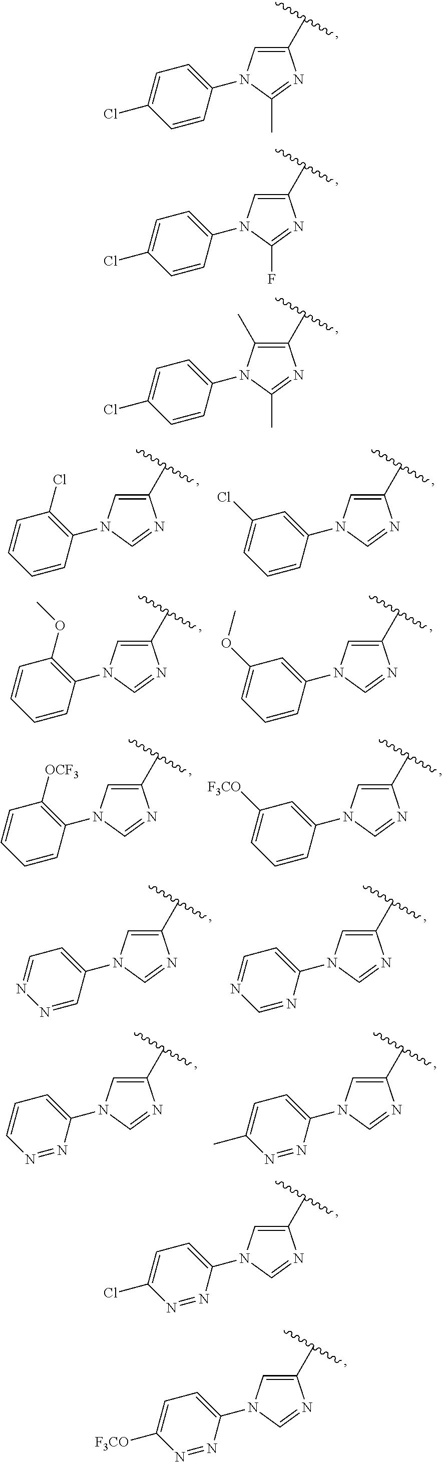

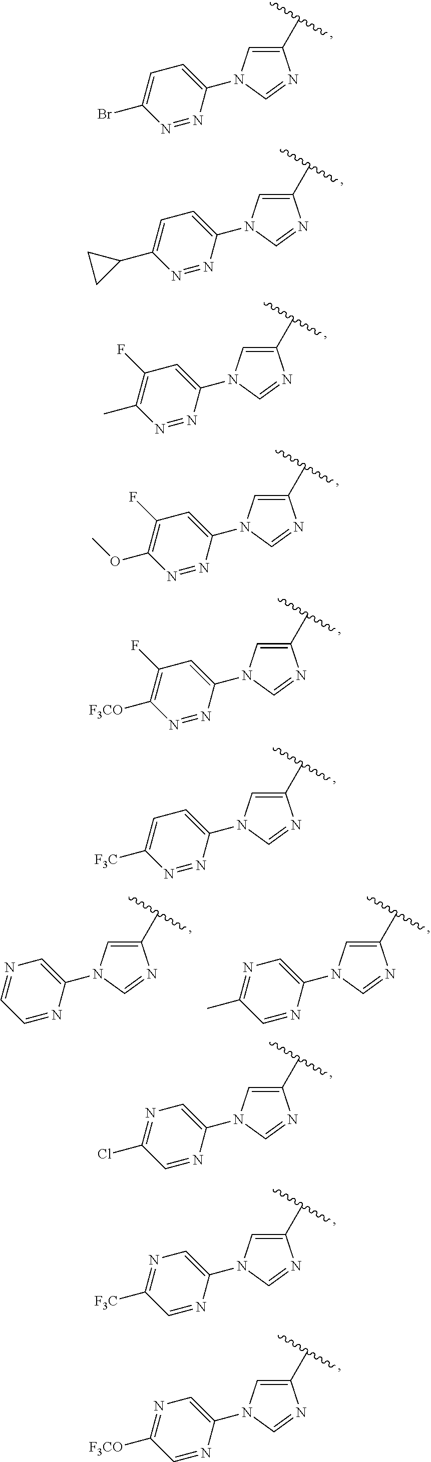

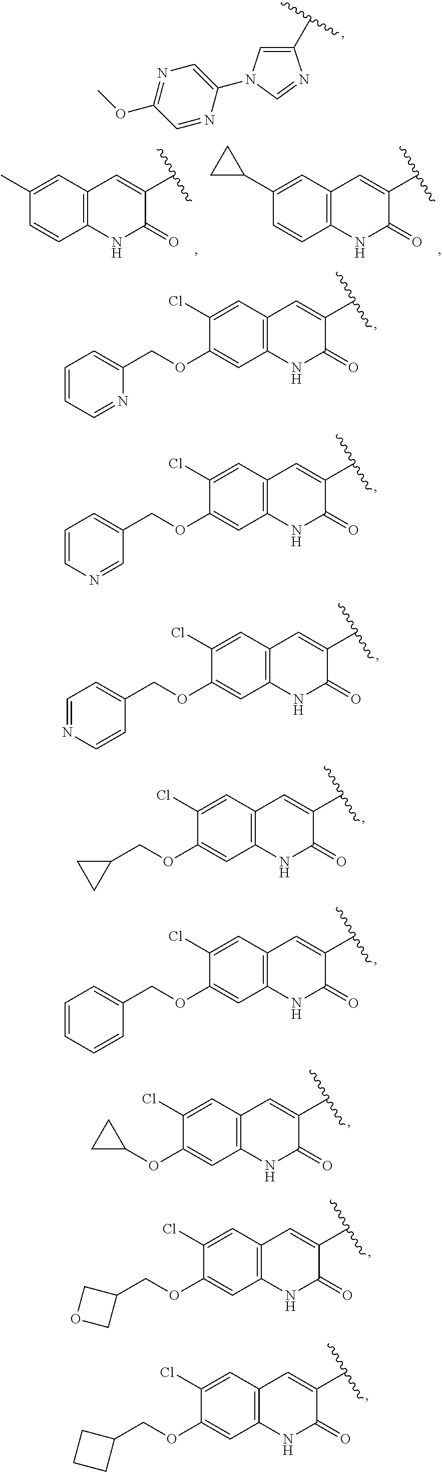

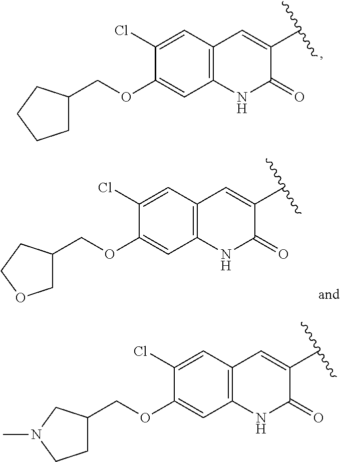 [SMILES: *c1cc2cc(Cl)c(OCC3CCCC3)cc2[nH]c1=O ; *c1cc2cc(Cl)c(OCC3CCOC3)cc2[nH]c1=O ; *c1cc2cc(Cl)c(OCC3CCN(C)C3)cc2[nH]c1=O]

wherein the wavy lines denote attachment points to rest of the molecule.

It is understood that each description of X, A, B, L, R¹, R², R³, R⁴, R⁵, R^{6a}, R^{6b}, R⁷ and R⁷′ may be independently combined with each description of X, A, B, L, R¹, R², R³, R⁴, R⁵, R^{6a}, R^{6b}, R⁷ and R^{7′} the same as if each and every combination were specifically and individually listed.

In some embodiments, the compound of formula (IA) is a compound of the formula (I):

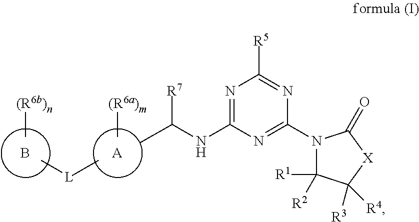

or a salt, polymorph, solvate, enantiomer, stereoisomer or tautomer thereof, wherein

wherein,

X is O, S or NR^{a};

R^{a} is hydrogen or C₁-C₆ alkyl optionally substituted by oxo, —OH or halogen;

A is C₆-C₁₀ aryl, 5- to 10-membered heteroaryl, C₃-C₈ cycloalkyl or 3- to 10-membered heterocyclyl, wherein each of which is optionally substituted by R^{6a};

B is hydrogen, C₆-C₁₀ aryl, 5- to 10-membered heteroaryl, C₃-C₈ cycloalkyl or 3- to 10-membered heterocyclyl, wherein each of which is optionally substituted by R^{6b};

L is a bond, —O—, —(CH₂)₁₋₃—, —NH—, —NCH₃—, —SO₂—, —C(O)—, —C(O)NH— or —NHC(O)—;

R¹ is hydrogen, halogen or C₁-C₆ alkyl;

R² is hydrogen, halogen, C₁-C₆ alkoxy, C₁-C₆ haloalkoxy, C₆ aryl, 5- to 6-membered heteroaryl or C₁-C₆ alkyl optionally substituted by oxo, halogen, —OR^{2a} or —NR^{2a}R^{2b};
R^{2a} and R^{2b} are independently hydrogen or C₁-C₆ alkyl;

or R¹ and R² are taken together with the atom to which they are attached to form a C₃-C₆ cycloalkyl or 3- to 6-membered heterocyclyl, each of which is optionally substituted by oxo, —OH, halogen, —NH₂, or C₁-C₆ alkyl optionally substituted by oxo, —OH, halogen or —NH₂;

R³ and R⁴ are independently hydrogen, halogen, or C₁-C₆ alkyl optionally substituted by oxo, —OH or halogen;

or R³ and R⁴ are taken together with the atom to which they are attached to form a C₃-C₆ cycloalkyl or 3- to 6 membered heterocyclyl, each of which is optionally substituted by oxo, —OH, -halogen, —NH₂, or C₁-C₆ alkyl optionally substituted by oxo, —OH, halogen or —NH₂;

R⁵ is hydrogen, C₁-C₆ alkyl, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, —OR¹⁰, —SR¹⁰, —S(O)₂R¹⁰, —S(O)₂NR¹¹R¹², —NR¹⁰S(O)₂R¹¹, —NR¹¹R¹², —C(O)R¹⁰, —NR¹⁰C(O)R¹¹, —NR¹⁰C(O)NR¹¹R¹², —C(O)OR¹⁰, —C(O)ONR¹¹R¹², —C(O)NR¹¹R¹², wherein each of which is optionally substituted by R⁸;

each R^{6a} and R^{6b} is independently oxo, C₁-C₆ alkyl, C₂-C₆ alkenyl, C₂-C₆ alkynyl, C₃-C₆ cycloalkyl, 3- to 6-membered heterocyclyl, —CN, halogen, C₁-C₆ alkoxy, C₁-C₆ haloalkoxy, C₁-C₆ haloalkyl, —OR¹³, —SR¹³, —S(O)₂R¹³, —S(O)₂NR¹⁴R¹⁵, —NR¹³S(O)₂R¹⁴, —NR¹⁴R¹⁵, —C(O)R¹³, —NR¹³C(O)R¹⁴, —NR¹³C(O)NR¹⁴R¹⁵, —C(O)OR¹³, —C(O)ONR¹⁴R¹⁵, —C(O)NR¹⁴R¹⁵, —(C₁-C₃ alkylene)OR¹³, —(C₁-C₃ alkylene)SR¹³, —(C₁—C₃ alkylene)S(O)₂R¹³, —(C₁-C₃ alkylene)S(O)₂NR¹⁴R¹⁵, —(C₁-C₃ alkylene)NR¹³S(O)₂R¹⁴, —(C₁-C₃ alkylene)NR¹⁴R¹⁵, —(C₁-C₃ alkylene)C(O)R¹³, —(C₁-C₃ alkylene)NR ¹³C(O)R¹⁴, —(C₁-C₃ alkylene)NR¹³C(O)NR¹⁴R¹⁵, —(C₁-C₃ alkylene)C(O)OR¹³, —(C₁-C₃ alkylene)C(O)ONR¹⁴R¹⁵, —(C₁-C₃ alkylene)(C₃-C₈ cycloalkyl) or —(C₁-C₃ alkylene)(3-10-membered heterocyclyl); wherein each of R^{6a} and R^{6b} is independently optionally substituted by oxo, C₂-C₆ alkenyl, C₂-C₆ alkynyl, —CN, halogen, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶, C₁-C₆ alkyl optionally substituted by oxo, OH, halogen or NH₂; m and n is independently 0, 1, 2, 3 or 4;

R⁷ is hydrogen or CH₃;

R⁸ is halogen, oxo, C₂-C₆ alkenyl, C₂-C₆ alkynyl, C₃-C₆ cycloalkyl or 3- to 6-membered heterocyclyl, —CN, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶ or C₁-C₆ alkyl optionally substituted by oxo, —OH, halogen or NH₂;

each R¹⁰, R¹¹ and R¹² is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹⁰, R¹¹ and R¹² is independently optionally substituted by oxo, C₂-C₆ alkenyl, C₂-C₆ alkynyl, —CN, halogen, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶ or C₁-C₆ alkyl optionally substituted by oxo, OH, halogen or NH₂;

or R¹¹ and R¹² are taken together with the atom to which they attached to form a 3-6 membered heterocyclyl optionally substituted by oxo, OH, halogen, NH₂, or C₁-C₆ alkyl optionally substituted by oxo, OH, halogen or NH₂;

each R¹³, R¹⁴ and R¹⁵ is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹³, R¹⁴ and R¹⁵ is independently optionally substituted by oxo, C₂-C₆ alkenyl, C₂-C₆ alkynyl, —CN, halogen, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶ or C₁-C₆ alkyl optionally substituted by oxo, OH, halogen or NH₂;

or R¹⁴ and R¹⁵ are taken together with the atom to which they attached to form a 3- to 6-membered heterocyclyl optionally substituted by oxo, OH or halogen, or C₁-C₆ alkyl optionally substituted by oxo, OH, halogen or NH₂;

each R¹⁶, R¹⁷ and R¹⁸ is independently hydrogen, C₂-C₆ alkenyl, C₂-C₆ alkynyl, or C₁-C₆ alkyl optionally substituted by oxo, OH, halogen or NH₂;

or R¹⁷ and R¹⁸ are taken together with the atom to which they attached to form a 3-6 membered heterocyclyl optionally substituted by oxo, OH, halogen or NH₂, or C₁-C₆ alkyl optionally substituted by oxo, OH, halogen or NH₂.

In some embodiments, a compound of formula (IA) is a compound of any of the compounds of formula (Ia-1) to (Ia-14),

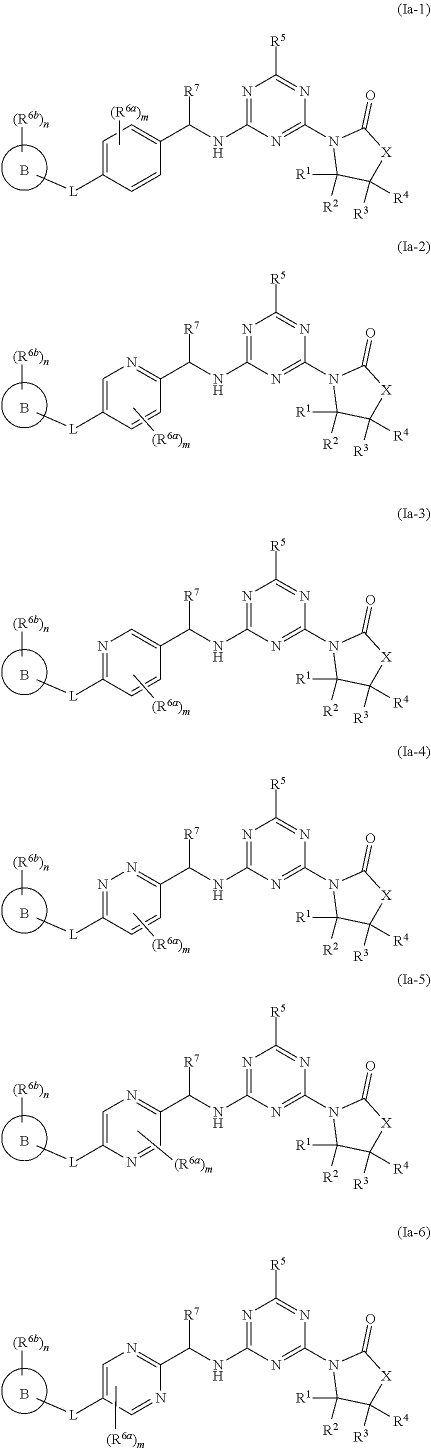

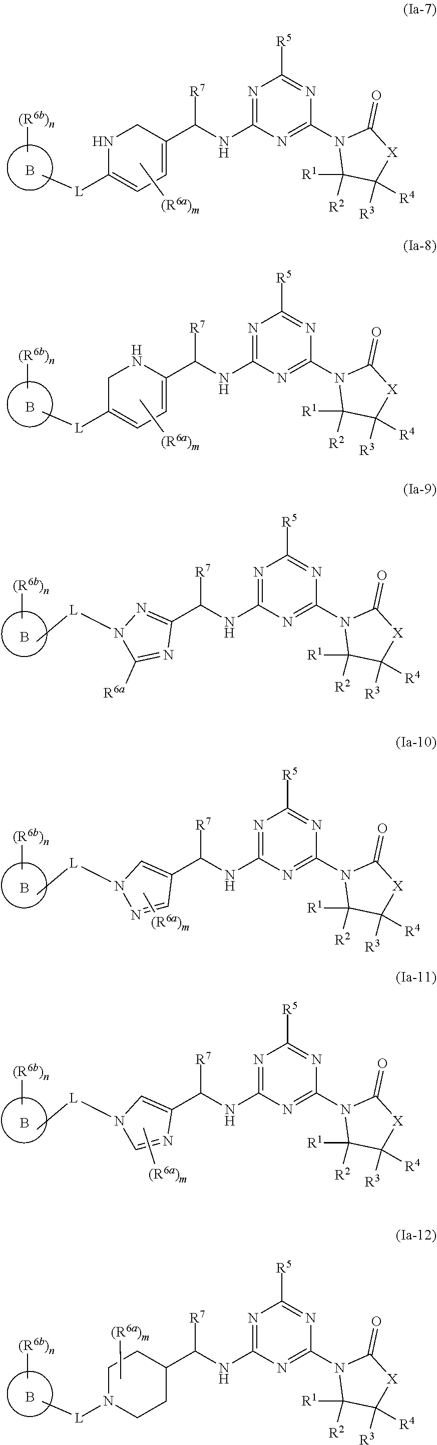

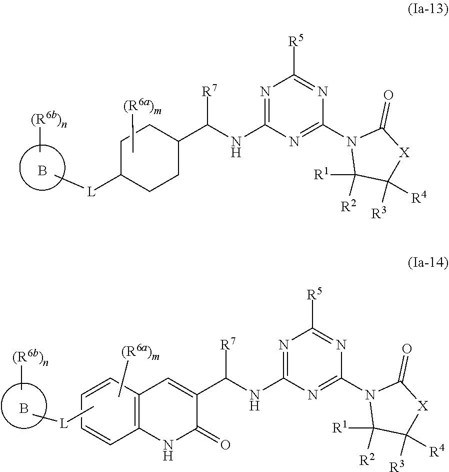

or a salt, polymorph, solvate, enantiomer, stereoisomer or tautomer thereof, wherein X, B, L, R¹, R², R³, R⁴, R⁵, R^{6a}, R^{6b}, R⁷, m and n are as defined for formula (IA).

In some embodiments, a compound of formula (IA) is a compound of any of the compounds of formula (Ib-1) to (Ib-11),

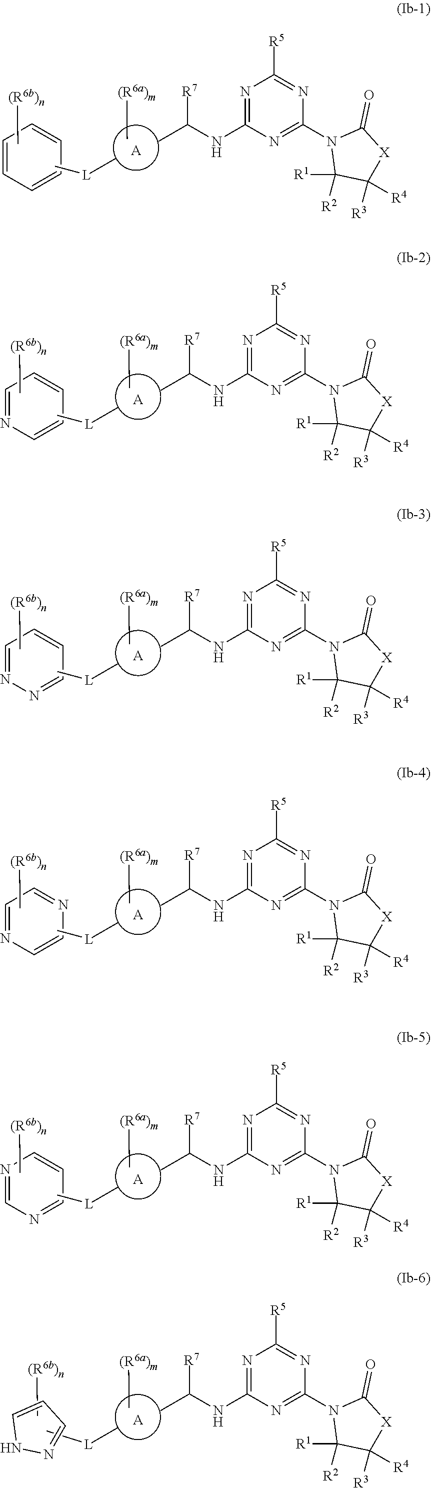

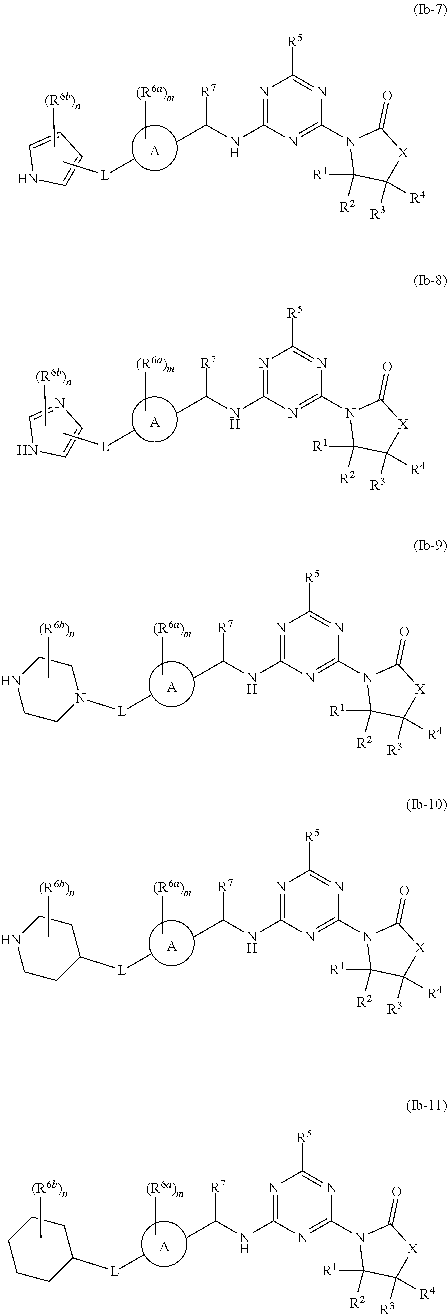

or a salt, polymorph, solvate, enantiomer, stereoisomer or tautomer thereof, wherein X, A, L, R¹, R², R³, R⁴, R⁵, R^{6a}, R^{6b}, R⁷, m and n are as defined for formula (IA).

In some embodiments, a compound of formula (IA) is a compound of formula (II),

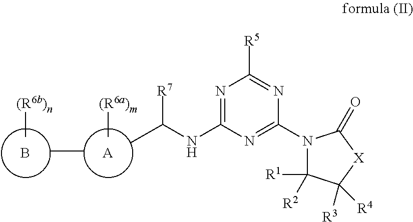

or a salt, polymorph, solvate, enantiomer, stereoisomer or tautomer thereof, wherein X, A, B, R¹, R², R³, R⁴, R⁵, R^{6a}, R^{6b}, R⁷, m and n are as defined for formula (IA).

In some embodiments, a compound of formula (IA) is a compound of any of the compounds of formula (IIa-1) to (IIa-8),

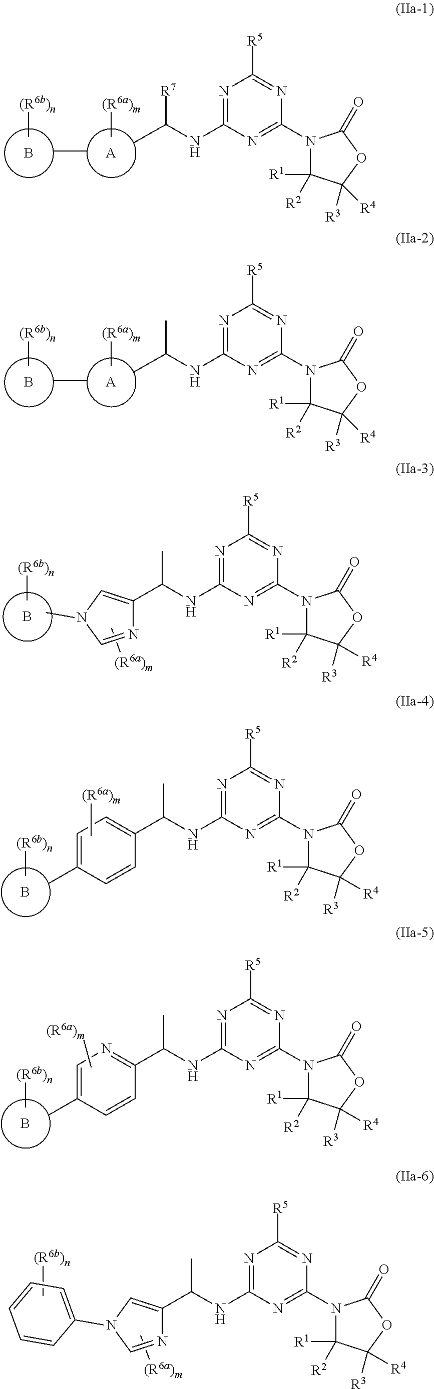

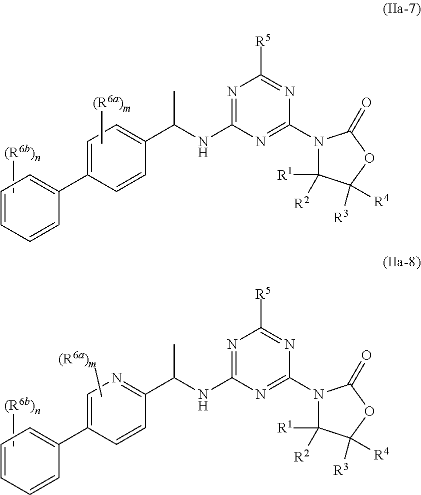

or a salt, polymorph, solvate, enantiomer, stereoisomer or tautomer thereof, wherein A, B, R¹, R², R³, R⁴, R⁵, R^{6a}, R^{6b}, R⁷, m and n are as defined for formula (IA).

In some embodiments, a compound of formula (IA) is a compound of formula (III),

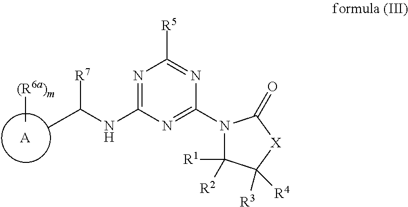

or a salt, polymorph, solvate, enantiomer, stereoisomer or tautomer thereof, wherein X, A, R¹, R², R³, R⁴, R⁵, R^{6a}, R⁷ and m are as defined for formula (IA).

In some embodiments, a compound of formula (IA) is a compound of any of the compounds of formula (IIIa-1) to (IIIa-8),

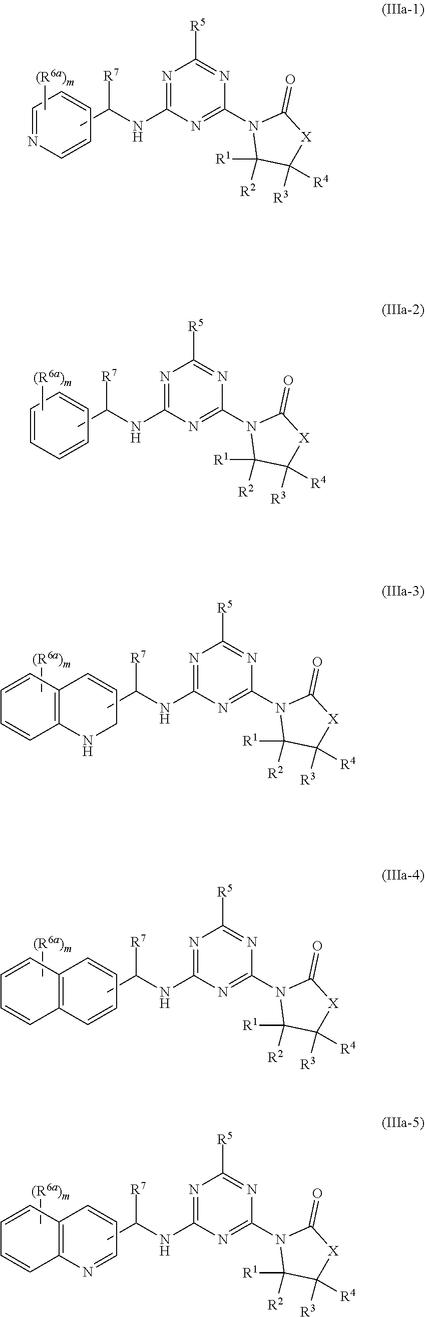

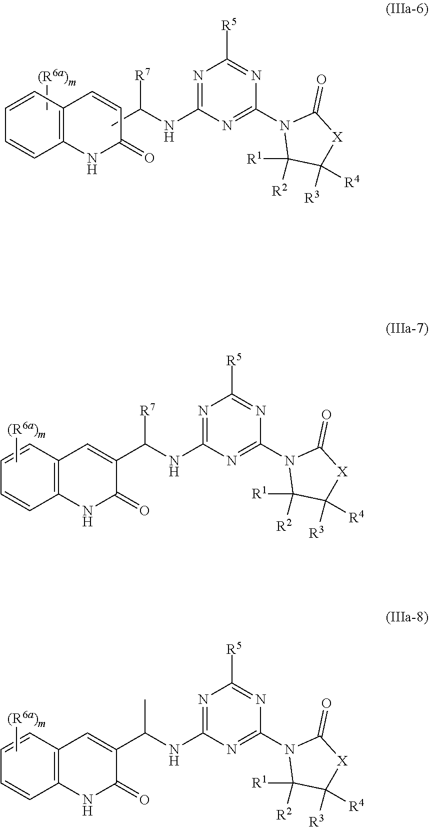

or a salt, polymorph, solvate, enantiomer, stereoisomer or tautomer thereof, wherein X, R¹, R², R³, R⁴, R⁵, R^{6a}, R⁷ and m are as defined for formula (IA). When A ring is bicyclic, any one ring or both rings may be substituted by the same or different R^{6a}.

In some embodiments, a compound of formula (IA) is a compound of formula (IV),

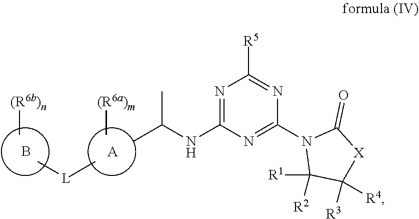

or a salt, polymorph, solvate, enantiomer, stereoisomer or tautomer thereof, wherein X, A, B, L, R¹, R², R³, R⁴, R⁵, R^{6a}, R^{6b}, m and n are as defined for formula (IA).

In some embodiments, a compound of formula (IA) is a compound of any of the compounds of formula (IVa-1) to (IVa-7),

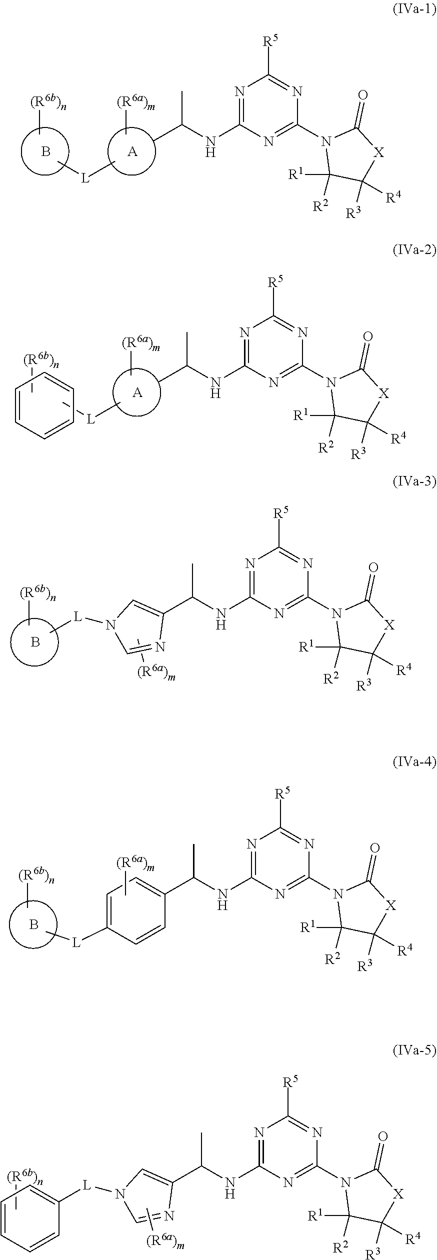

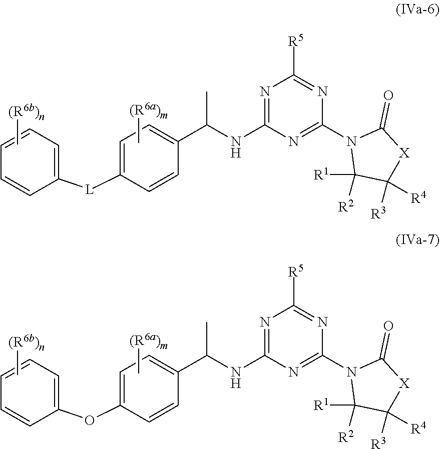

or a salt, polymorph, solvate, enantiomer, stereoisomer or tautomer thereof, wherein A, B, L, R¹, R², R³, R⁴, R⁵, R^{6a}, R^{6b}, m and n are as defined for formula (IA).

In some embodiments, a compound of formula (IA) is a compound of formula (V),

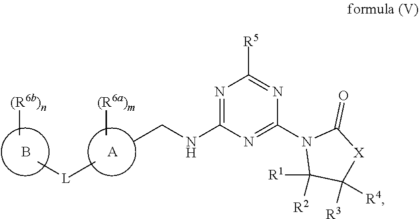

or a salt, polymorph, solvate, enantiomer, stereoisomer or tautomer thereof, wherein X, A, B, L, R¹, R², R³, R⁴, R⁵, R^{6a}, R^{6b}, m and n are as defined for formula (IA).

In some embodiments, a compound of formula (IA) is a compound of formula (VI),

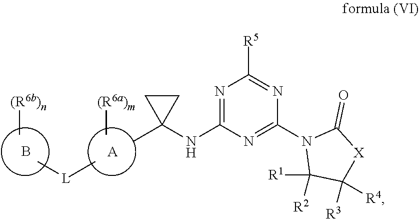

or a salt, polymorph, solvate, enantiomer, stereoisomer or tautomer thereof, wherein X, A, B, L, R¹, R², R³, R⁴, R⁵, R^{6a}, R^{6b}, m and n are as defined for formula (IA).

Also provided are salts of compounds referred to herein, such as pharmaceutically acceptable salts. The invention also includes any or all of the stereochemical forms, including any enantiomeric or diastereomeric forms, and any tautomers or other forms of the compounds described.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Representative compounds of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)),) are listed in table-1 and table-2. It is understood that individual enantiomers and diastereomers are included in the generic compound structures shown in table-1 and table-2. Specific synthetic methods for preparing compounds of table-1 are provided example herein.

TABLE 1
Compounds

| | |
|---|---|
| 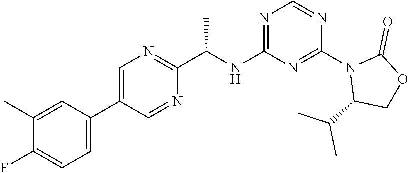 [SMILES: Cc1cc(-c2cnc([C@H](C)Nc3ncnc(N4C(=O)OC[C@@H]4C(C)C)n3)nc2)ccc1F] | 1.1 |
| 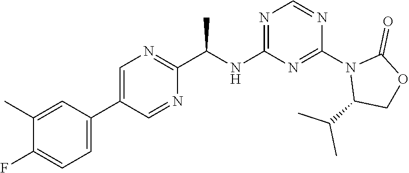 [SMILES: Cc1cc(-c2cnc([C@@H](C)Nc3ncnc(N4C(=O)OC[C@@H]4C(C)C)n3)nc2)ccc1F] | 1.2 |
| 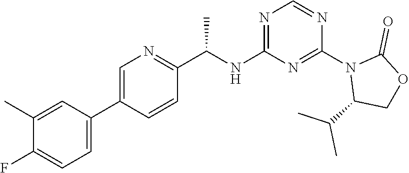 [SMILES: Cc1cc(-c2ccc([C@H](C)Nc3ncnc(N4C(=O)OC[C@@H]4C(C)C)n3)nc2)ccc1F] | 1.3 |
| 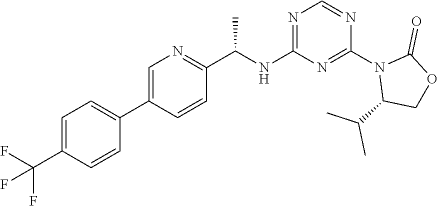 [SMILES: CC(C)[C@H]1COC(=O)N1c1ncnc(N[C@@H](C)c2ccc(-c3ccc(C(F)(F)F)cc3)cn2)n1] | 1.4 |
| 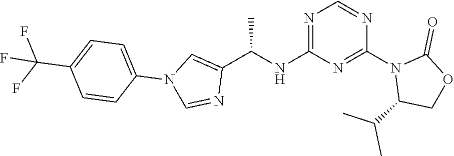 [SMILES: CC(C)[C@H]1COC(=O)N1c1ncnc(N[C@@H](C)c2cn(-c3ccc(C(F)(F)F)cc3)cn2)n1] | 1.5 |
| 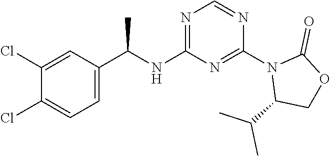 [SMILES: CC(C)[C@H]1COC(=O)N1c1ncnc(N[C@H](C)c2ccc(Cl)c(Cl)c2)n1] | 1.6 |
| 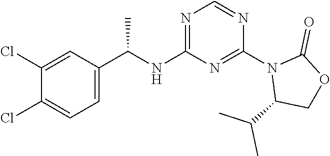 [SMILES: CC(C)[C@H]1COC(=O)N1c1ncnc(N[C@@H](C)c2ccc(Cl)c(Cl)c2)n1] | 1.7 |
| 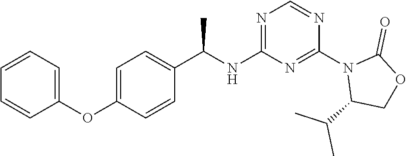 [SMILES: CC(C)[C@H]1COC(=O)N1c1ncnc(N[C@H](C)c2ccc(Oc3ccccc3)cc2)n1] | 1.8 |
| 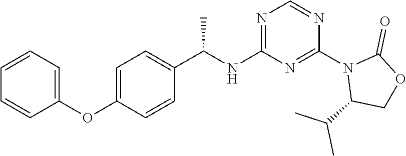 [SMILES: CC(C)[C@H]1COC(=O)N1c1ncnc(N[C@@H](C)c2ccc(Oc3ccccc3)cc2)n1] | 1.9 |
| 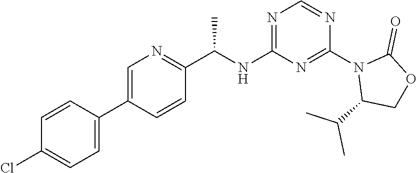 [SMILES: CC(C)[C@H]1COC(=O)N1c1ncnc(N[C@@H](C)c2ccc(-c3ccc(Cl)cc3)cn2)n1] | 1.10 |
| 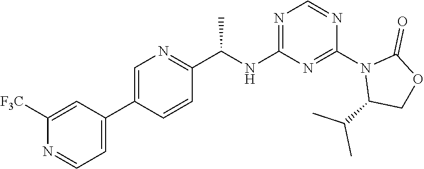 [SMILES: CC(C)[C@H]1COC(=O)N1c1ncnc(N[C@@H](C)c2ccc(-c3ccnc(C(F)(F)F)c3)cn2)n1] | 1.11 |
| 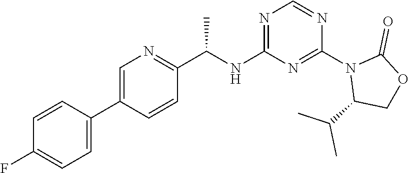 [SMILES: CC(C)[C@H]1COC(=O)N1c1ncnc(N[C@@H](C)c2ccc(-c3ccc(F)cc3)cn2)n1] | 1.12 |
| 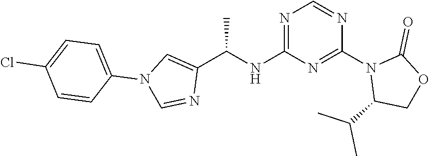 [SMILES: CC(C)[C@H]1COC(=O)N1c1ncnc(N[C@@H](C)c2cn(-c3ccc(Cl)cc3)cn2)n1] | 1.13 |
| 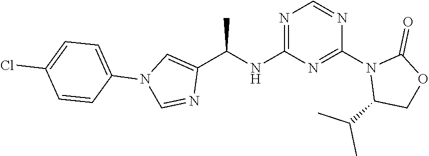 [SMILES: CC(C)[C@H]1COC(=O)N1c1ncnc(N[C@H](C)c2cn(-c3ccc(Cl)cc3)cn2)n1] | 1.14 |
| 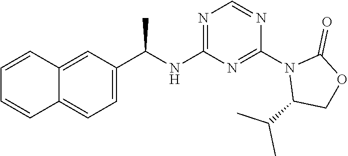 [SMILES: CC(C)[C@H]1COC(=O)N1c1ncnc(N[C@H](C)c2ccc3ccccc3c2)n1] | 1.15 |
| 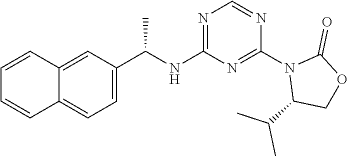 [SMILES: CC(C)[C@H]1COC(=O)N1c1ncnc(N[C@@H](C)c2ccc3ccccc3c2)n1] | 1.16 |
| 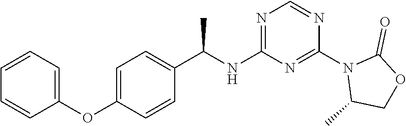 [SMILES: C[C@@H](Nc1ncnc(N2C(=O)OC[C@@H]2C)n1)c1ccc(Oc2ccccc2)cc1] | 1.17 |
| 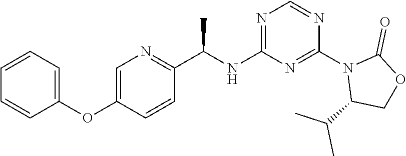 [SMILES: CC(C)[C@H]1COC(=O)N1c1ncnc(N[C@H](C)c2ccc(Oc3ccccc3)cn2)n1] | 1.18 |
| 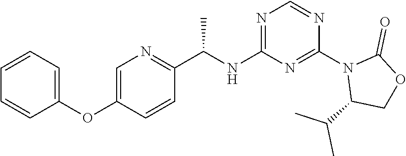 [SMILES: CC(C)[C@H]1COC(=O)N1c1ncnc(N[C@@H](C)c2ccc(Oc3ccccc3)cn2)n1] | 1.19 |
| 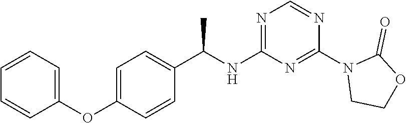 [SMILES: C[C@@H](Nc1ncnc(N2CCOC2=O)n1)c1ccc(Oc2ccccc2)cc1] | 1.20 |
| 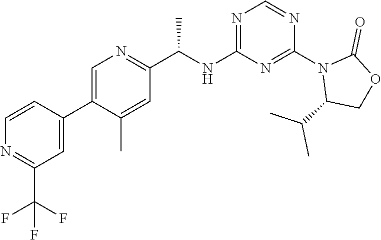 [SMILES: Cc1cc([C@H](C)Nc2ncnc(N3C(=O)OC[C@@H]3C(C)C)n2)ncc1-c1ccnc(C(F)(F)F)c1] | 1.21 |
| 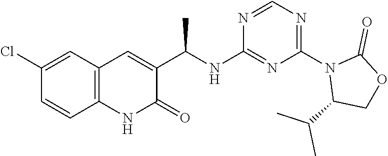 [SMILES: CC(C)[C@H]1COC(=O)N1c1ncnc(N[C@H](C)c2cc3cc(Cl)ccc3[nH]c2=O)n1] | 1.22 |
| 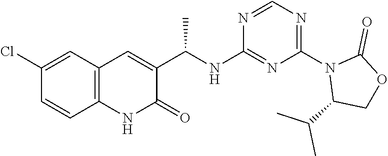 [SMILES: CC(C)[C@H]1COC(=O)N1c1ncnc(N[C@@H](C)c2cc3cc(Cl)ccc3[nH]c2=O)n1] | 1.23 |
| 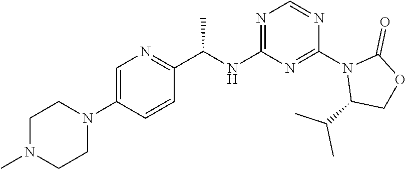 [SMILES: CC(C)[C@H]1COC(=O)N1c1ncnc(N[C@@H](C)c2ccc(N3CCN(C)CC3)cn2)n1] | 1.24 |
| 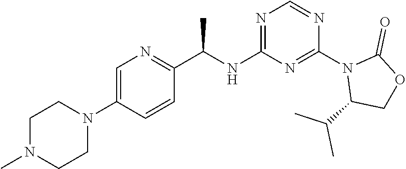 [SMILES: CC(C)[C@H]1COC(=O)N1c1ncnc(N[C@H](C)c2ccc(N3CCN(C)CC3)cn2)n1] | 1.25 |
| 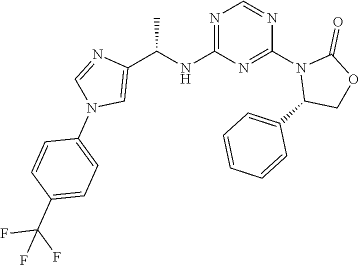 [SMILES: C[C@H](Nc1ncnc(N2C(=O)OC[C@@H]2c2ccccc2)n1)c1cn(-c2ccc(C(F)(F)F)cc2)cn1] | 1.26 |
| 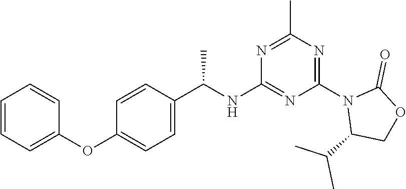 [SMILES: Cc1nc(N[C@@H](C)c2ccc(Oc3ccccc3)cc2)nc(N2C(=O)OC[C@@H]2C(C)C)n1] | 1.27 |
| 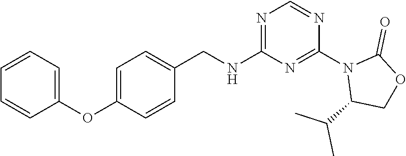 [SMILES: CC(C)[C@H]1COC(=O)N1c1ncnc(NCc2ccc(Oc3ccccc3)cc2)n1] | 1.28 |
| 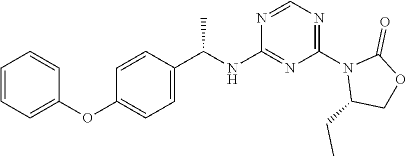 [SMILES: CC[C@H]1COC(=O)N1c1ncnc(N[C@@H](C)c2ccc(Oc3ccccc3)cc2)n1] | 1.29 |
| 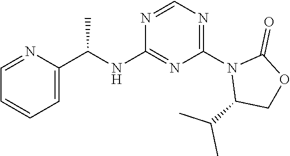 [SMILES: CC(C)[C@H]1COC(=O)N1c1ncnc(N[C@@H](C)c2ccccn2)n1] | 1.30 |
| 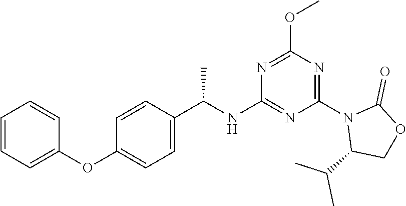 [SMILES: COc1nc(N[C@@H](C)c2ccc(Oc3ccccc3)cc2)nc(N2C(=O)OC[C@@H]2C(C)C)n1] | 1.31 |
| 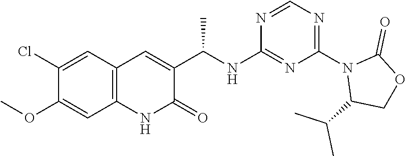 | 1.32 |
| 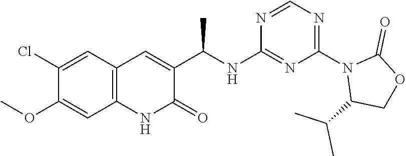 | 1.33 |
| 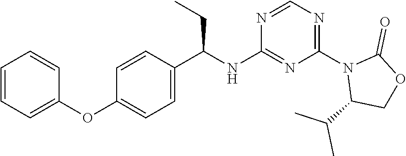 [SMILES: CC[C@@H](Nc1ncnc(N2C(=O)OC[C@@H]2C(C)C)n1)c1ccc(Oc2ccccc2)cc1] | 1.34 |
| 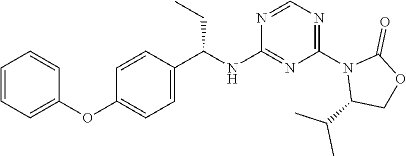 [SMILES: CC[C@H](Nc1ncnc(N2C(=O)OC[C@@H]2C(C)C)n1)c1ccc(Oc2ccccc2)cc1] | 1.35 |
| 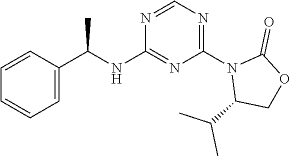 [SMILES: CC(C)[C@H]1COC(=O)N1c1ncnc(N[C@H](C)c2ccccc2)n1] | 1.36 |
| 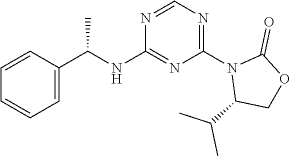 [SMILES: CC(C)[C@H]1COC(=O)N1c1ncnc(N[C@@H](C)c2ccccc2)n1] | 1.37 |
| 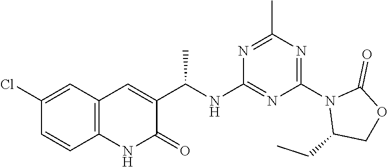 [SMILES: CC[C@H]1COC(=O)N1c1nc(C)nc(N[C@@H](C)c2cc3cc(Cl)ccc3[nH]c2=O)n1] | 1.38 |
| 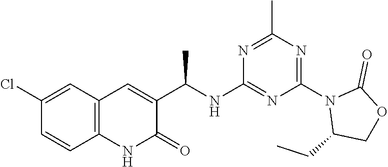 [SMILES: CC[C@H]1COC(=O)N1c1nc(C)nc(N[C@H](C)c2cc3cc(Cl)ccc3[nH]c2=O)n1] | 1.39 |
| 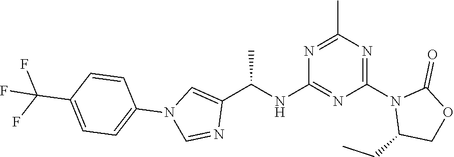 [SMILES: CC[C@H]1COC(=O)N1c1nc(C)nc(N[C@@H](C)c2cn(-c3ccc(C(F)(F)F)cc3)cn2)n1] | 1.40 |
| 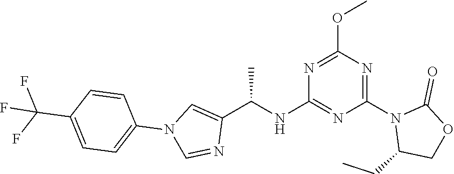 [SMILES: CC[C@H]1COC(=O)N1c1nc(N[C@@H](C)c2cn(-c3ccc(C(F)(F)F)cc3)cn2)nc(OC)n1] | 1.41 |
| 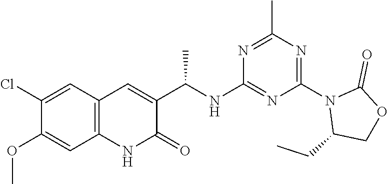 [SMILES: CC[C@H]1COC(=O)N1c1nc(C)nc(N[C@@H](C)c2cc3cc(Cl)c(OC)cc3[nH]c2=O)n1] | 1.42 |
| 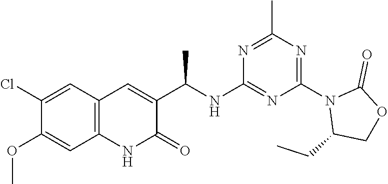 [SMILES: CC[C@H]1COC(=O)N1c1nc(C)nc(N[C@H](C)c2cc3cc(Cl)c(OC)cc3[nH]c2=O)n1] | 1.43 |
| 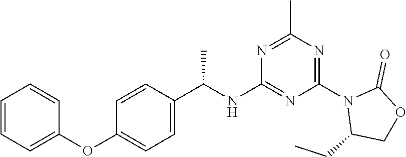 [SMILES: CC[C@H]1COC(=O)N1c1nc(C)nc(N[C@@H](C)c2ccc(Oc3ccccc3)cc2)n1] | 1.44 |
| 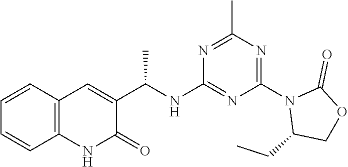 [SMILES: CC[C@H]1COC(=O)N1c1nc(C)nc(N[C@@H](C)c2cc3ccccc3[nH]c2=O)n1] | 1.45 |
| 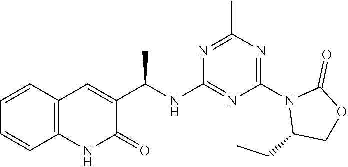 [SMILES: CC[C@H]1COC(=O)N1c1nc(C)nc(N[C@H](C)c2cc3ccccc3[nH]c2=O)n1] | 1.46 |
| 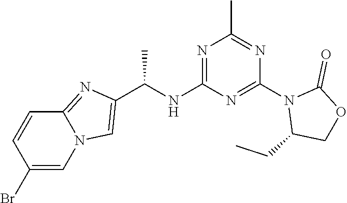 [SMILES: CC[C@H]1COC(=O)N1c1nc(C)nc(N[C@@H](C)c2cn3cc(Br)ccc3n2)n1] | 1.47 |
| 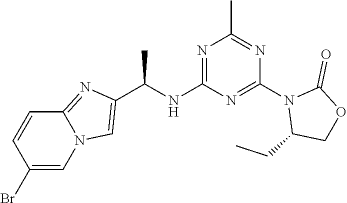 [SMILES: CC[C@H]1COC(=O)N1c1nc(C)nc(N[C@H](C)c2cn3cc(Br)ccc3n2)n1] | 1.48 |
| 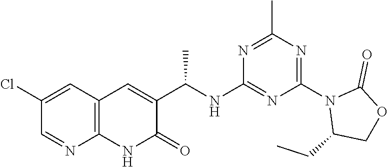 [SMILES: CC[C@H]1COC(=O)N1c1nc(C)nc(N[C@@H](C)c2cc3cc(Cl)cnc3[nH]c2=O)n1] | 1.49 |
| 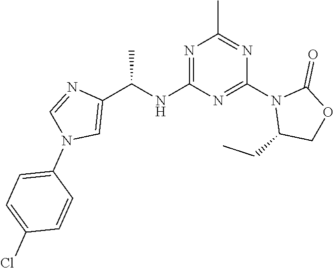 [SMILES: CC[C@H]1COC(=O)N1c1nc(C)nc(N[C@@H](C)c2cn(-c3ccc(Cl)cc3)cn2)n1] | 1.50 |
| 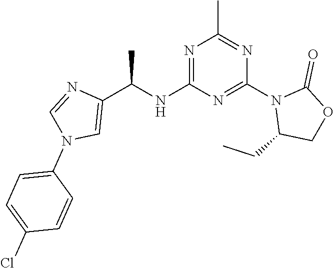 [SMILES: CC[C@H]1COC(=O)N1c1nc(C)nc(N[C@H](C)c2cn(-c3ccc(Cl)cc3)cn2)n1] | 1.51 |
| 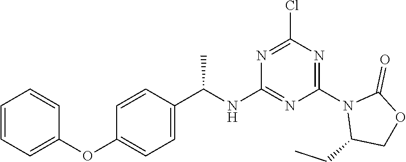 [SMILES: CC[C@H]1COC(=O)N1c1nc(Cl)nc(N[C@@H](C)c2ccc(Oc3ccccc3)cc2)n1] | 1.52 |
| 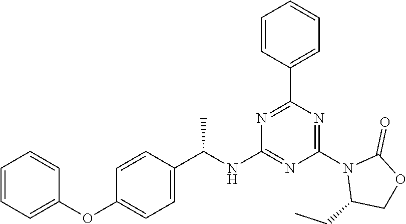 [SMILES: CC[C@H]1COC(=O)N1c1nc(N[C@@H](C)c2ccc(Oc3ccccc3)cc2)nc(-c2ccccc2)n1] | 1.53 |
| 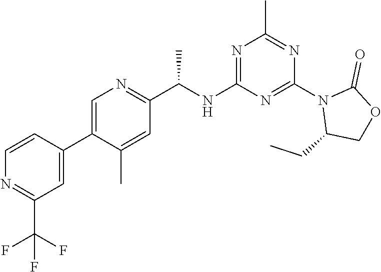 [SMILES: CC[C@H]1COC(=O)N1c1nc(C)nc(N[C@@H](C)c2cc(C)c(-c3ccnc(C(F)(F)F)c3)cn2)n1] | 1.54 |
| 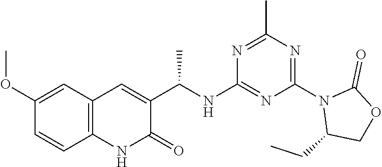 [SMILES: CC[C@H]1COC(=O)N1c1nc(C)nc(N[C@@H](C)c2cc3cc(OC)ccc3[nH]c2=O)n1] | 1.55 |
| 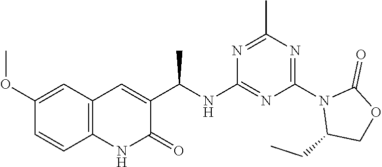 [SMILES: CC[C@H]1COC(=O)N1c1nc(C)nc(N[C@H](C)c2cc3cc(OC)ccc3[nH]c2=O)n1] | 1.56 |
| 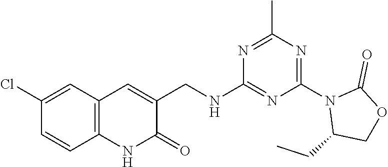 [SMILES: CC[C@H]1COC(=O)N1c1nc(C)nc(NCc2cc3cc(Cl)ccc3[nH]c2=O)n1] | 1.57 |
| 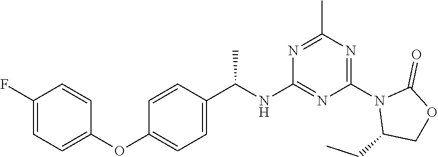 [SMILES: CC[C@H]1COC(=O)N1c1nc(C)nc(N[C@@H](C)c2ccc(Oc3ccc(F)cc3)cc2)n1] | 1.58 |
| 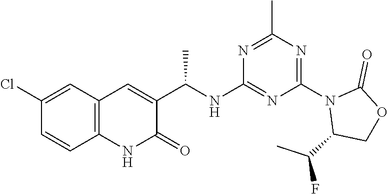 [SMILES: Cc1nc(N[C@@H](C)c2cc3cc(Cl)ccc3[nH]c2=O)nc(N2C(=O)OC[C@@H]2[C@H](C)F)n1] | 1.59 |
| 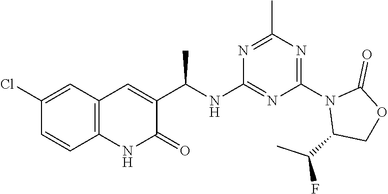 [SMILES: Cc1nc(N[C@H](C)c2cc3cc(Cl)ccc3[nH]c2=O)nc(N2C(=O)OC[C@@H]2[C@H](C)F)n1] | 1.60 |
| 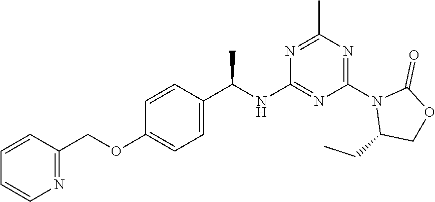 [SMILES: CC[C@H]1COC(=O)N1c1nc(C)nc(N[C@H](C)c2ccc(OCc3ccccn3)cc2)n1] | 1.61 |
| 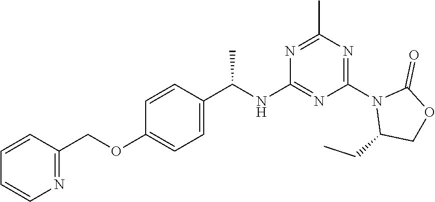 [SMILES: CC[C@H]1COC(=O)N1c1nc(C)nc(N[C@@H](C)c2ccc(OCc3ccccn3)cc2)n1] | 1.62 |
| 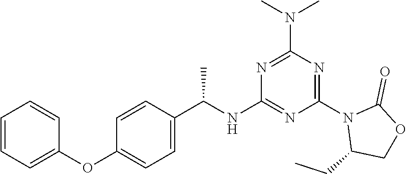 [SMILES: CC[C@H]1COC(=O)N1c1nc(N[C@@H](C)c2ccc(Oc3ccccc3)cc2)nc(N(C)C)n1] | 1.63 |
| 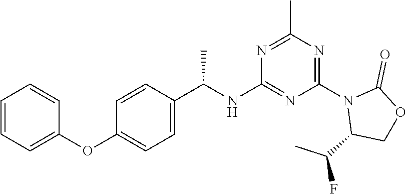 [SMILES: Cc1nc(N[C@@H](C)c2ccc(Oc3ccccc3)cc2)nc(N2C(=O)OC[C@@H]2[C@H](C)F)n1] | 1.64 |
| 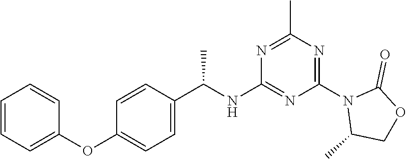 [SMILES: Cc1nc(N[C@@H](C)c2ccc(Oc3ccccc3)cc2)nc(N2C(=O)OC[C@@H]2C)n1] | 1.65 |
| 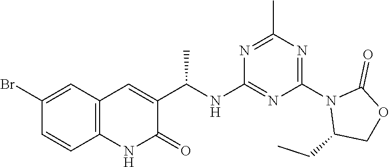 [SMILES: CC[C@H]1COC(=O)N1c1nc(C)nc(N[C@@H](C)c2cc3cc(Br)ccc3[nH]c2=O)n1] | 1.66 |
| 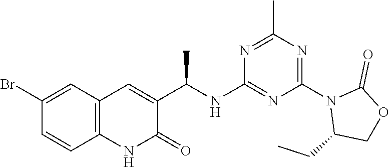 [SMILES: CC[C@H]1COC(=O)N1c1nc(C)nc(N[C@H](C)c2cc3cc(Br)ccc3[nH]c2=O)n1] | 1.67 |
| 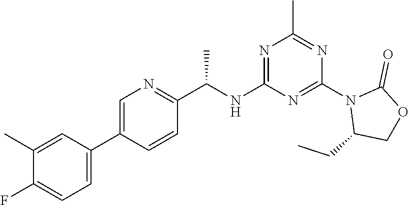 [SMILES: CC[C@H]1COC(=O)N1c1nc(C)nc(N[C@@H](C)c2ccc(-c3ccc(F)c(C)c3)cn2)n1] | 1.68 |
| 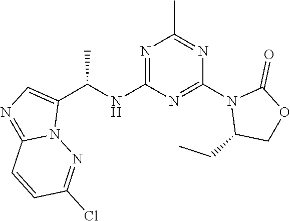 [SMILES: CC[C@H]1COC(=O)N1c1nc(C)nc(N[C@@H](C)c2cnc3ccc(Cl)nn23)n1] | 1.69 |
| 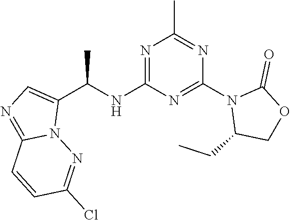 [SMILES: CC[C@H]1COC(=O)N1c1nc(C)nc(N[C@H](C)c2cnc3ccc(Cl)nn23)n1] | 1.70 |
| 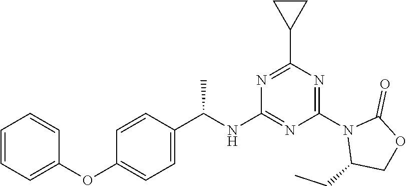 [SMILES: CC[C@H]1COC(=O)N1c1nc(N[C@@H](C)c2ccc(Oc3ccccc3)cc2)nc(C2CC2)n1] | 1.71 |
| 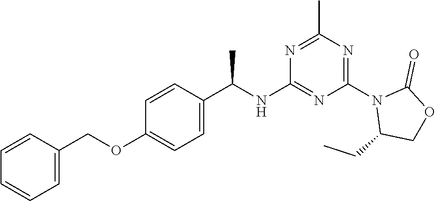 [SMILES: CC[C@H]1COC(=O)N1c1nc(C)nc(N[C@H](C)c2ccc(OCc3ccccc3)cc2)n1] | 1.72 |
| 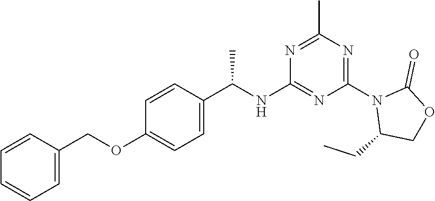 [SMILES: CC[C@H]1COC(=O)N1c1nc(C)nc(N[C@@H](C)c2ccc(OCc3ccccc3)cc2)n1] | 1.73 |
| 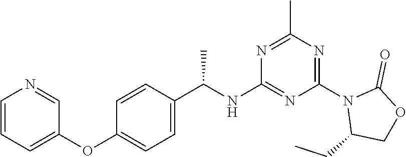 [SMILES: CC[C@H]1COC(=O)N1c1nc(C)nc(N[C@@H](C)c2ccc(Oc3cccnc3)cc2)n1] | 1.74 |
| 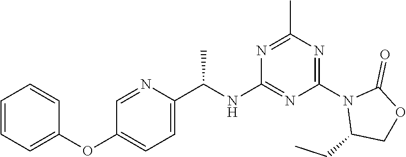 [SMILES: CC[C@H]1COC(=O)N1c1nc(C)nc(N[C@@H](C)c2ccc(Oc3ccccc3)cn2)n1] | 1.75 |
| 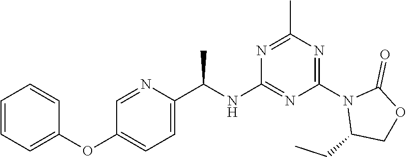 [SMILES: CC[C@H]1COC(=O)N1c1nc(C)nc(N[C@H](C)c2ccc(Oc3ccccc3)cn2)n1] | 1.76 |
| 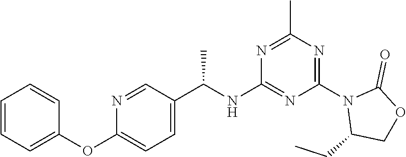 [SMILES: CC[C@H]1COC(=O)N1c1nc(C)nc(N[C@@H](C)c2ccc(Oc3ccccc3)nc2)n1] | 1.77 |
| 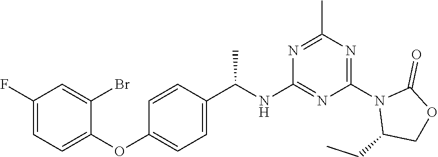 [SMILES: CC[C@H]1COC(=O)N1c1nc(C)nc(N[C@@H](C)c2ccc(Oc3ccc(F)cc3Br)cc2)n1] | 1.78 |
| 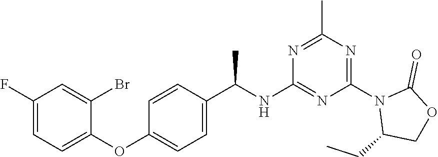 [SMILES: CC[C@H]1COC(=O)N1c1nc(C)nc(N[C@H](C)c2ccc(Oc3ccc(F)cc3Br)cc2)n1] | 1.79 |
| 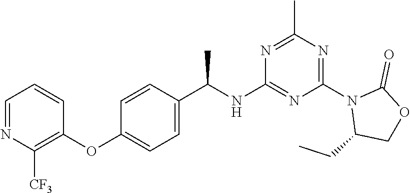 [SMILES: CC[C@H]1COC(=O)N1c1nc(C)nc(N[C@H](C)c2ccc(Oc3cccnc3C(F)(F)F)cc2)n1] | 1.80 |
| 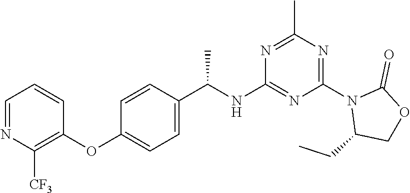 [SMILES: CC[C@H]1COC(=O)N1c1nc(C)nc(N[C@@H](C)c2ccc(Oc3cccnc3C(F)(F)F)cc2)n1] | 1.81 |
| 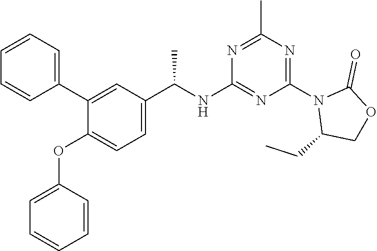 [SMILES: CC[C@H]1COC(=O)N1c1nc(C)nc(N[C@@H](C)c2ccc(Oc3ccccc3)c(-c3ccccc3)c2)n1] | 1.82 |
| 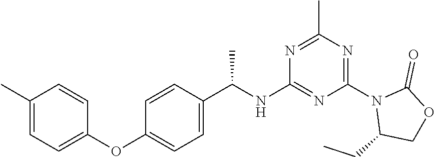 [SMILES: CC[C@H]1COC(=O)N1c1nc(C)nc(N[C@@H](C)c2ccc(Oc3ccc(C)cc3)cc2)n1] | 1.83 |
| 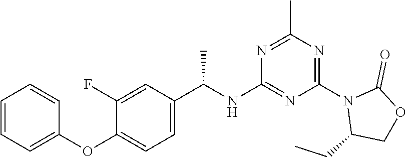 [SMILES: CC[C@H]1COC(=O)N1c1nc(C)nc(N[C@@H](C)c2ccc(Oc3ccccc3)c(F)c2)n1] | 1.84 |
| 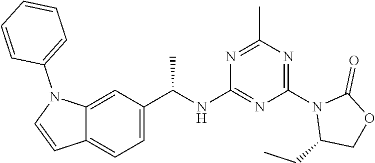 [SMILES: CC[C@H]1COC(=O)N1c1nc(C)nc(N[C@@H](C)c2ccc3ccn(-c4ccccc4)c3c2)n1] | 1.85 |
| 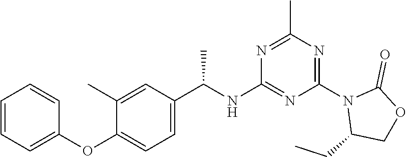 [SMILES: CC[C@H]1COC(=O)N1c1nc(C)nc(N[C@@H](C)c2ccc(Oc3ccccc3)c(C)c2)n1] | 1.86 |
| 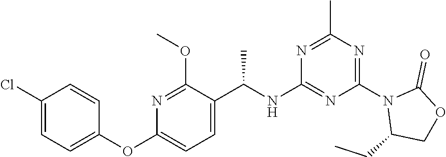 [SMILES: CC[C@H]1COC(=O)N1c1nc(C)nc(N[C@@H](C)c2ccc(Oc3ccc(Cl)cc3)nc2OC)n1] | 1.87 |
| 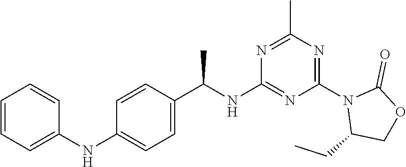 [SMILES: CC[C@H]1COC(=O)N1c1nc(C)nc(N[C@H](C)c2ccc(Nc3ccccc3)cc2)n1] | 1.88 |
| 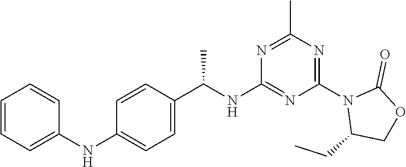 [SMILES: CC[C@H]1COC(=O)N1c1nc(C)nc(N[C@@H](C)c2ccc(Nc3ccccc3)cc2)n1] | 1.89 |
| 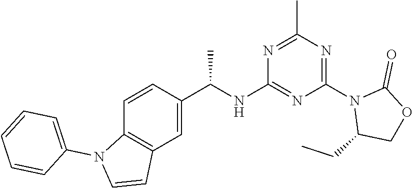 [SMILES: CC[C@H]1COC(=O)N1c1nc(C)nc(N[C@@H](C)c2ccc3c(ccn3-c3ccccc3)c2)n1] | 1.90 |
| 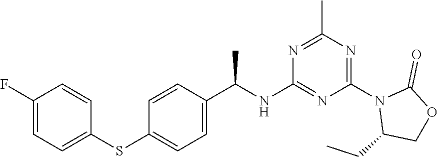 [SMILES: CC[C@H]1COC(=O)N1c1nc(C)nc(N[C@H](C)c2ccc(Sc3ccc(F)cc3)cc2)n1] | 1.91 |
| 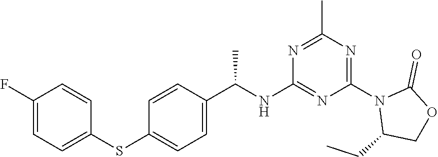 [SMILES: CC[C@H]1COC(=O)N1c1nc(C)nc(N[C@@H](C)c2ccc(Sc3ccc(F)cc3)cc2)n1] | 1.92 |
| 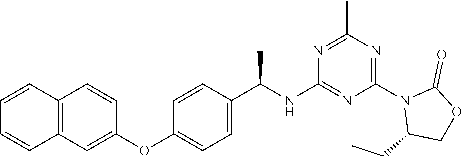 [SMILES: CC[C@H]1COC(=O)N1c1nc(C)nc(N[C@H](C)c2ccc(Oc3ccc4ccccc4c3)cc2)n1] | 1.93 |
| 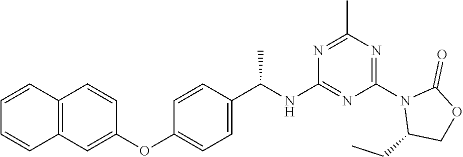 [SMILES: CC[C@H]1COC(=O)N1c1nc(C)nc(N[C@@H](C)c2ccc(Oc3ccc4ccccc4c3)cc2)n1] | 1.94 |
| 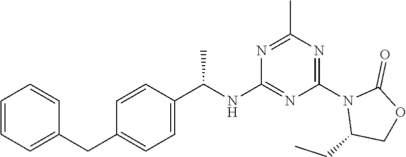 [SMILES: CC[C@H]1COC(=O)N1c1nc(C)nc(N[C@@H](C)c2ccc(Cc3ccccc3)cc2)n1] | 1.95 |
| 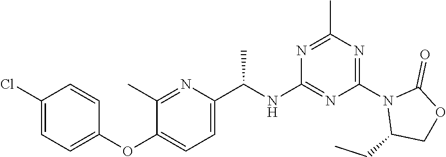 [SMILES: CC[C@H]1COC(=O)N1c1nc(C)nc(N[C@@H](C)c2ccc(Oc3ccc(Cl)cc3)c(C)n2)n1] | 1.96 |
| 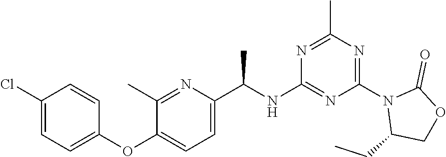 [SMILES: CC[C@H]1COC(=O)N1c1nc(C)nc(N[C@H](C)c2ccc(Oc3ccc(Cl)cc3)c(C)n2)n1] | 1.97 |
| 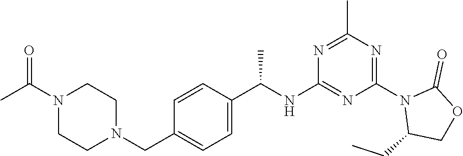 [SMILES: CC[C@H]1COC(=O)N1c1nc(C)nc(N[C@@H](C)c2ccc(CN3CCN(C(C)=O)CC3)cc2)n1] | 1.98 |
| 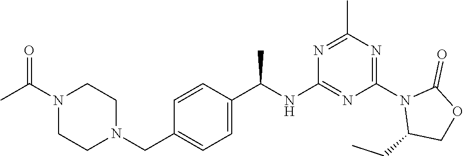 [SMILES: CC[C@H]1COC(=O)N1c1nc(C)nc(N[C@H](C)c2ccc(CN3CCN(C(C)=O)CC3)cc2)n1] | 1.99 |
| 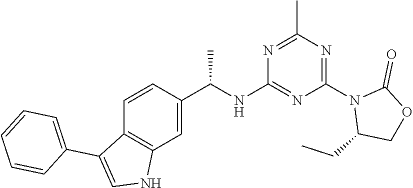 [SMILES: CC[C@H]1COC(=O)N1c1nc(C)nc(N[C@@H](C)c2ccc3c(-c4ccccc4)c[nH]c3c2)n1] | 1.100 |
| 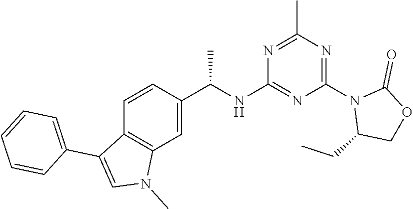 [SMILES: CC[C@H]1COC(=O)N1c1nc(C)nc(N[C@@H](C)c2ccc3c(-c4ccccc4)cn(C)c3c2)n1] | 1.101 |
| 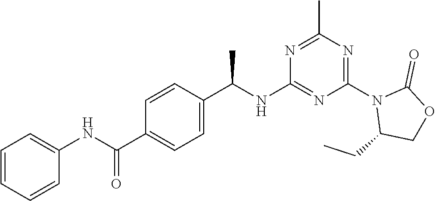 [SMILES: CC[C@H]1COC(=O)N1c1nc(C)nc(N[C@H](C)c2ccc(C(=O)Nc3ccccc3)cc2)n1] | 1.102 |
| 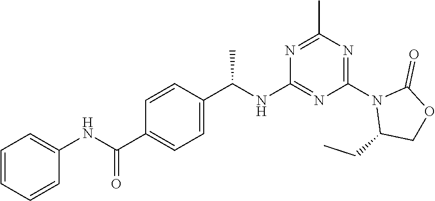 [SMILES: CC[C@H]1COC(=O)N1c1nc(C)nc(N[C@@H](C)c2ccc(C(=O)Nc3ccccc3)cc2)n1] | 1.103 |
| 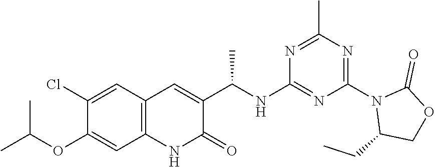 [SMILES: CC[C@H]1COC(=O)N1c1nc(C)nc(N[C@@H](C)c2cc3cc(Cl)c(OC(C)C)cc3[nH]c2=O)n1] | 1.104 |
| 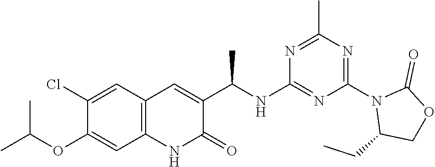 [SMILES: CC[C@H]1COC(=O)N1c1nc(C)nc(N[C@H](C)c2cc3cc(Cl)c(OC(C)C)cc3[nH]c2=O)n1] | 1.105 |
| 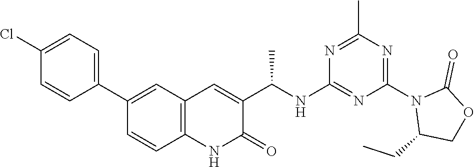 [SMILES: CC[C@H]1COC(=O)N1c1nc(C)nc(N[C@@H](C)c2cc3cc(-c4ccc(Cl)cc4)ccc3[nH]c2=O)n1] | 1.106 |
| 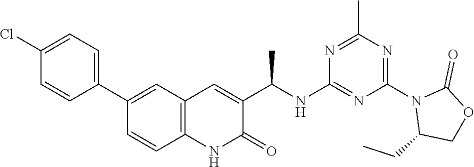 [SMILES: CC[C@H]1COC(=O)N1c1nc(C)nc(N[C@H](C)c2cc3cc(-c4ccc(Cl)cc4)ccc3[nH]c2=O)n1] | 1.107 |
| 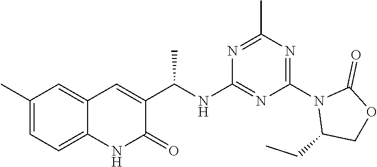 [SMILES: CC[C@H]1COC(=O)N1c1nc(C)nc(N[C@@H](C)c2cc3cc(C)ccc3[nH]c2=O)n1] | 1.108 |
| 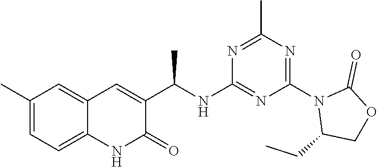 [SMILES: CC[C@H]1COC(=O)N1c1nc(C)nc(N[C@H](C)c2cc3cc(C)ccc3[nH]c2=O)n1] | 1.109 |
| 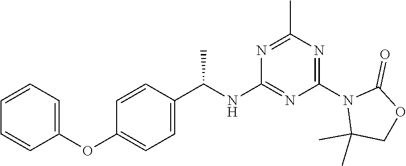 [SMILES: Cc1nc(N[C@@H](C)c2ccc(Oc3ccccc3)cc2)nc(N2C(=O)OCC2(C)C)n1] | 1.110 |
| 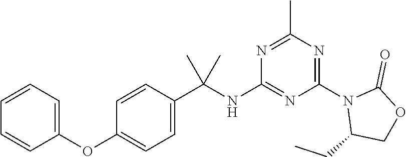 [SMILES: CC[C@H]1COC(=O)N1c1nc(C)nc(NC(C)(C)c2ccc(Oc3ccccc3)cc2)n1] | 1.111 |
| 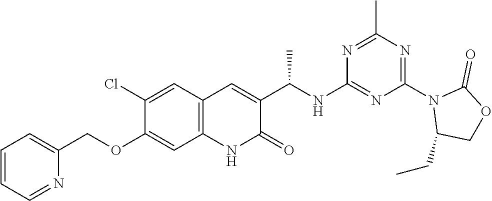 [SMILES: CC[C@H]1COC(=O)N1c1nc(C)nc(N[C@@H](C)c2cc3cc(Cl)c(OCc4ccccn4)cc3[nH]c2=O)n1] | 1.112 |
| 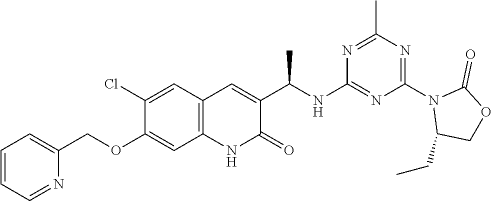 [SMILES: CC[C@H]1COC(=O)N1c1nc(C)nc(N[C@H](C)c2cc3cc(Cl)c(OCc4ccccn4)cc3[nH]c2=O)n1] | 1.113 |

The compounds illustrated in table-2 can be prepared in a manner analogous to the techniques used in connection with the preparation of the table-1 compounds and in accordance, using appropriate, analogous starting materials and by utilizing the general synthetic schemes illustrated below.

TABLE 2
Compounds

| | |
|---|---|
| 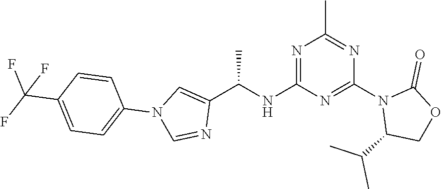 [SMILES: Cc1nc(N[C@@H](C)c2cn(-c3ccc(C(F)(F)F)cc3)cn2)nc(N2C(=O)OC[C@@H]2C(C)C)n1] | 2.1 |
| 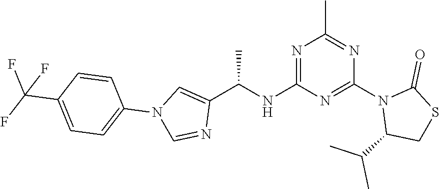 [SMILES: Cc1nc(N[C@@H](C)c2cn(-c3ccc(C(F)(F)F)cc3)cn2)nc(N2C(=O)SC[C@@H]2C(C)C)n1] | 2.2 |
| 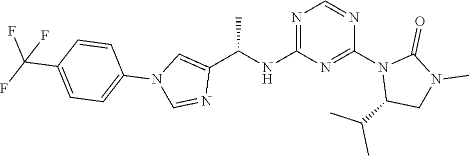 [SMILES: CC(C)[C@H]1CN(C)C(=O)N1c1ncnc(N[C@@H](C)c2cn(-c3ccc(C(F)(F)F)cc3)cn2)n1] | 2.3 |
| 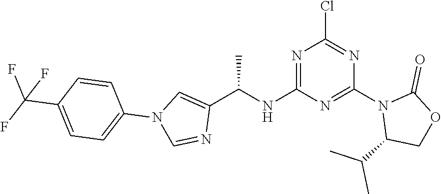 [SMILES: CC(C)[C@H]1COC(=O)N1c1nc(Cl)nc(N[C@@H](C)c2cn(-c3ccc(C(F)(F)F)cc3)cn2)n1] | 2.4 |
| 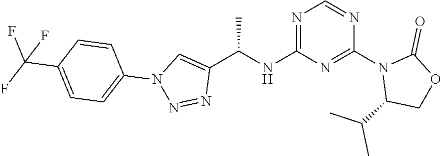 [SMILES: CC(C)[C@H]1COC(=O)N1c1ncnc(N[C@@H](C)c2cn(-c3ccc(C(F)(F)F)cc3)nn2)n1] | 2.5 |
| 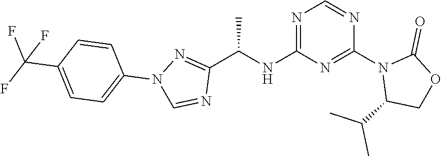 [SMILES: CC(C)[C@H]1COC(=O)N1c1ncnc(N[C@@H](C)c2ncn(-c3ccc(C(F)(F)F)cc3)n2)n1] | 2.6 |
| 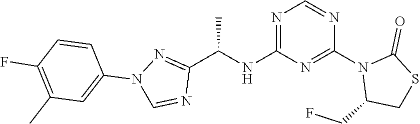 [SMILES: Cc1cc(-n2cnc([C@H](C)Nc3ncnc(N4C(=O)SC[C@@H]4CF)n3)n2)ccc1F] | 2.7 |
| 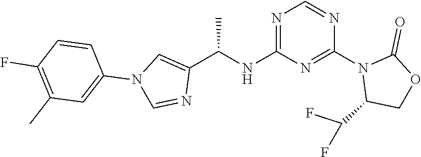 [SMILES: Cc1cc(-n2cnc([C@H](C)Nc3ncnc(N4C(=O)OC[C@@H]4C(F)F)n3)c2)ccc1F] | 2.8 |
| 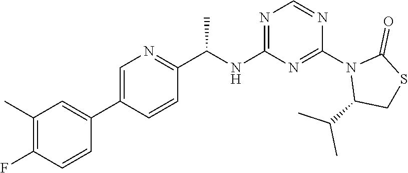 [SMILES: Cc1cc(-c2ccc([C@H](C)Nc3ncnc(N4C(=O)SC[C@@H]4C(C)C)n3)nc2)ccc1F] | 2.9 |
| 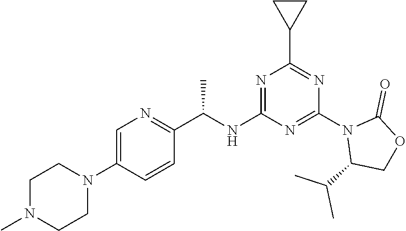 [SMILES: CC(C)[C@H]1COC(=O)N1c1nc(N[C@@H](C)c2ccc(N3CCN(C)CC3)cn2)nc(C2CC2)n1] | 2.10 |
| 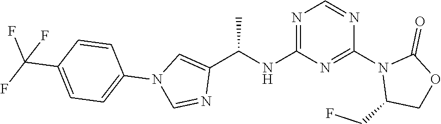 [SMILES: C[C@H](Nc1ncnc(N2C(=O)OC[C@@H]2CF)n1)c1cn(-c2ccc(C(F)(F)F)cc2)cn1] | 2.11 |
| 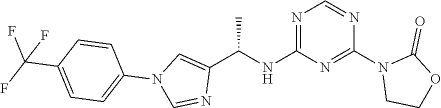 [SMILES: C[C@H](Nc1ncnc(N2CCOC2=O)n1)c1cn(-c2ccc(C(F)(F)F)cc2)cn1] | 2.12 |
| 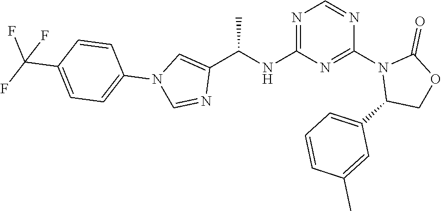 [SMILES: Cc1cccc([C@H]2COC(=O)N2c2ncnc(N[C@@H](C)c3cn(-c4ccc(C(F)(F)F)cc4)cn3)n2)c1] | 2.13 |
| 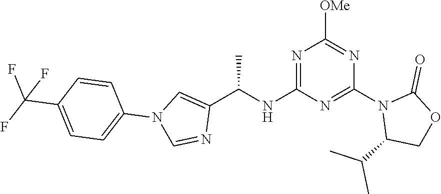 [SMILES: COc1nc(N[C@@H](C)c2cn(-c3ccc(C(F)(F)F)cc3)cn2)nc(N2C(=O)OC[C@@H]2C(C)C)n1] | 2.14 |
| 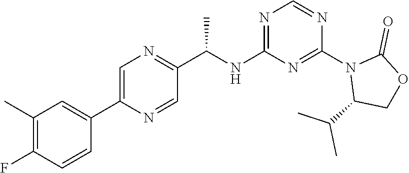 [SMILES: Cc1cc(-c2cnc([C@H](C)Nc3ncnc(N4C(=O)OC[C@@H]4C(C)C)n3)cn2)ccc1F] | 2.15 |
| 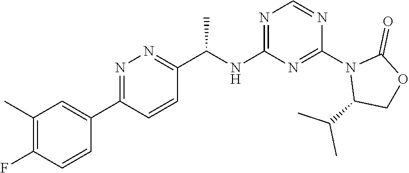 [SMILES: Cc1cc(-c2ccc([C@H](C)Nc3ncnc(N4C(=O)OC[C@@H]4C(C)C)n3)nn2)ccc1F] | 2.16 |
| 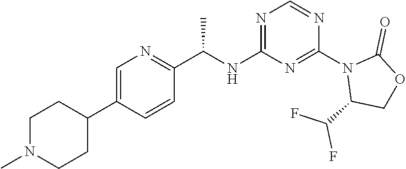 [SMILES: C[C@H](Nc1ncnc(N2C(=O)OC[C@@H]2C(F)F)n1)c1ccc(C2CCN(C)CC2)cn1] | 2.17 |
| 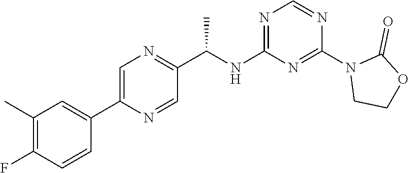 [SMILES: Cc1cc(-c2cnc([C@H](C)Nc3ncnc(N4CCOC4=O)n3)cn2)ccc1F] | 2.18 |
| 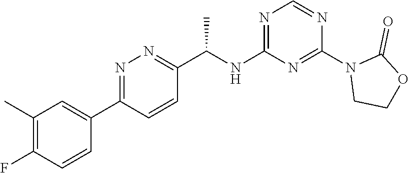 [SMILES: Cc1cc(-c2ccc([C@H](C)Nc3ncnc(N4CCOC4=O)n3)nn2)ccc1F] | 2.19 |
| 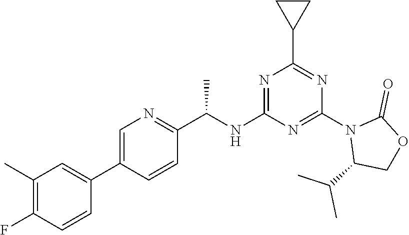 [SMILES: Cc1cc(-c2ccc([C@H](C)Nc3nc(C4CC4)nc(N4C(=O)OC[C@@H]4C(C)C)n3)nc2)ccc1F] | 2.20 |
| 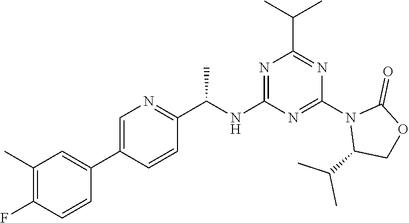 [SMILES: Cc1cc(-c2ccc([C@H](C)Nc3nc(C(C)C)nc(N4C(=O)OC[C@@H]4C(C)C)n3)nc2)ccc1F] | 2.21 |
| 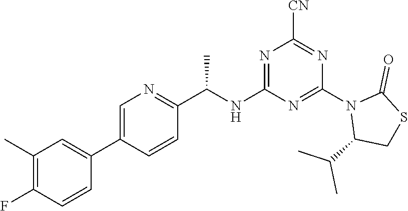 [SMILES: Cc1cc(-c2ccc([C@H](C)Nc3nc(C#N)nc(N4C(=O)SC[C@@H]4C(C)C)n3)nc2)ccc1F] | 2.22 |
| 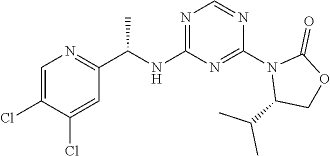 [SMILES: CC(C)[C@H]1COC(=O)N1c1ncnc(N[C@@H](C)c2cc(Cl)c(Cl)cn2)n1] | 2.23 |
| 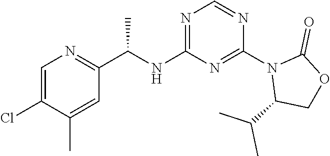 [SMILES: Cc1cc([C@H](C)Nc2ncnc(N3C(=O)OC[C@@H]3C(C)C)n2)ncc1Cl] | 2.24 |
| 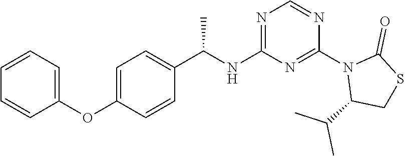 [SMILES: CC(C)[C@H]1CSC(=O)N1c1ncnc(N[C@@H](C)c2ccc(Oc3ccccc3)cc2)n1] | 2.25 |
| 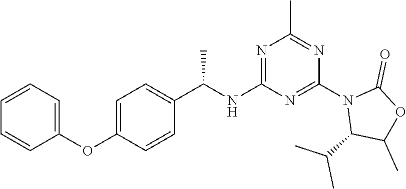 [SMILES: Cc1nc(N[C@@H](C)c2ccc(Oc3ccccc3)cc2)nc(N2C(=O)OC(C)[C@@H]2C(C)C)n1] | 2.26 |
| 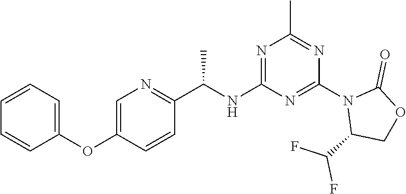 [SMILES: Cc1nc(N[C@@H](C)c2ccc(Oc3ccccc3)cn2)nc(N2C(=O)OC[C@@H]2C(F)F)n1] | 2.27 |
| 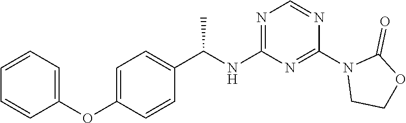 [SMILES: C[C@H](Nc1ncnc(N2CCOC2=O)n1)c1ccc(Oc2ccccc2)cc1] | 2.28 |
| 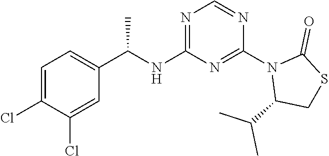 [SMILES: CC(C)[C@H]1CSC(=O)N1c1ncnc(N[C@@H](C)c2ccc(Cl)c(Cl)c2)n1] | 2.29 |
| 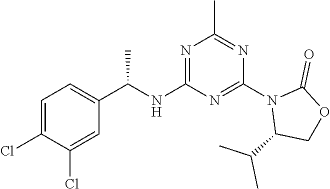 [SMILES: Cc1nc(N[C@@H](C)c2ccc(Cl)c(Cl)c2)nc(N2C(=O)OC[C@@H]2C(C)C)n1] | 2.30 |
| 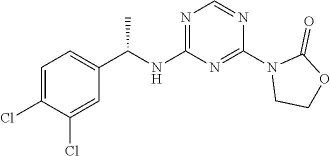 [SMILES: C[C@H](Nc1ncnc(N2CCOC2=O)n1)c1ccc(Cl)c(Cl)c1] | 2.31 |
| 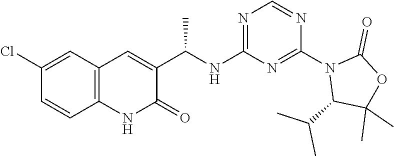 [SMILES: CC(C)[C@@H]1N(c2ncnc(N[C@@H](C)c3cc4cc(Cl)ccc4[nH]c3=O)n2)C(=O)OC1(C)C] | 2.32 |
| 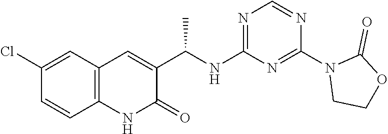 [SMILES: C[C@H](Nc1ncnc(N2CCOC2=O)n1)c1cc2cc(Cl)ccc2[nH]c1=O] | 2.33 |
| 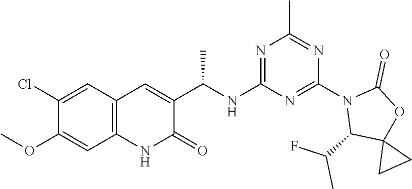 [SMILES: COc1cc2[nH]c(=O)c([C@H](C)Nc3nc(C)nc(N4C(=O)OC5(CC5)[C@@H]4C(C)F)n3)cc2cc1Cl] | 2.34 |
| 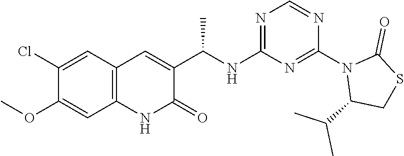 [SMILES: COc1cc2[nH]c(=O)c([C@H](C)Nc3ncnc(N4C(=O)SC[C@@H]4C(C)C)n3)cc2cc1Cl] | 2.35 |
| 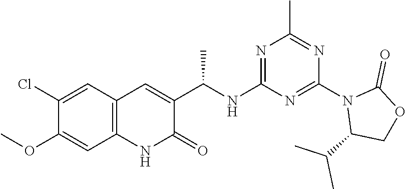 [SMILES: COc1cc2[nH]c(=O)c([C@H](C)Nc3nc(C)nc(N4C(=O)OC[C@@H]4C(C)C)n3)cc2cc1Cl] | 2.36 |
| 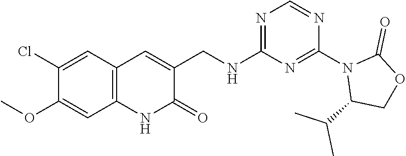 [SMILES: COc1cc2[nH]c(=O)c(CNc3ncnc(N4C(=O)OC[C@@H]4C(C)C)n3)cc2cc1Cl] | 2.37 |
| 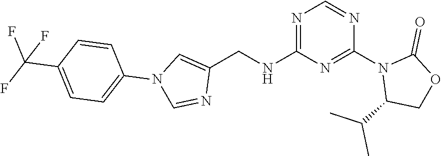 [SMILES: CC(C)[C@H]1COC(=O)N1c1ncnc(NCc2cn(-c3ccc(C(F)(F)F)cc3)cn2)n1] | 2.38 |
| 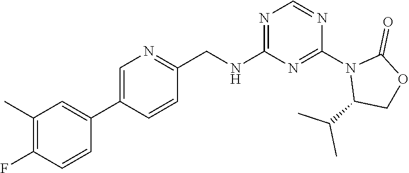 [SMILES: Cc1cc(-c2ccc(CNc3ncnc(N4C(=O)OC[C@@H]4C(C)C)n3)nc2)ccc1F] | 2.39 |
| 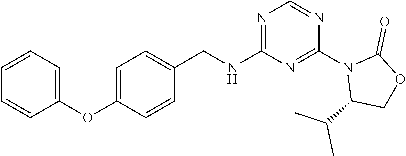 [SMILES: CC(C)[C@H]1COC(=O)N1c1ncnc(NCc2ccc(Oc3ccccc3)cc2)n1] | 2.40 |
| 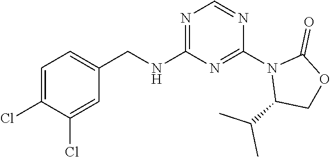 [SMILES: CC(C)[C@H]1COC(=O)N1c1ncnc(NCc2ccc(Cl)c(Cl)c2)n1] | 2.41 |
| 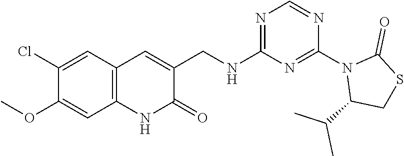 [SMILES: COc1cc2[nH]c(=O)c(CNc3ncnc(N4C(=O)SC[C@@H]4C(C)C)n3)cc2cc1Cl] | 2.42 |
| 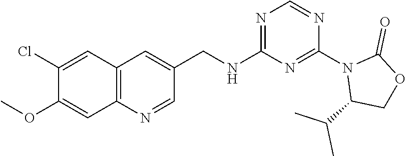 [SMILES: COc1cc2ncc(CNc3ncnc(N4C(=O)OC[C@@H]4C(C)C)n3)cc2cc1Cl] | 2.43 |
| 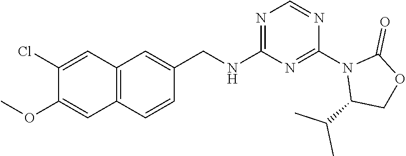 [SMILES: COc1cc2ccc(CNc3ncnc(N4C(=O)OC[C@@H]4C(C)C)n3)cc2cc1Cl] | 2.44 |
| 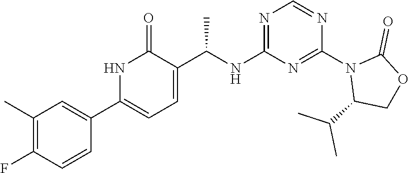 [SMILES: Cc1cc(-c2ccc([C@H](C)Nc3ncnc(N4C(=O)OC[C@@H]4C(C)C)n3)c(=O)[nH]2)ccc1F] | 2.45 |
| 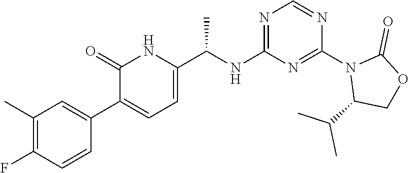 [SMILES: Cc1cc(-c2ccc([C@H](C)Nc3ncnc(N4C(=O)OC[C@@H]4C(C)C)n3)[nH]c2=O)ccc1F] | 2.46 |
| 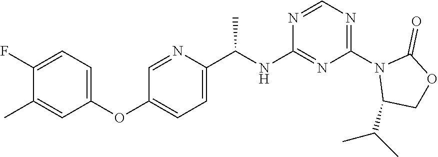 [SMILES: Cc1cc(Oc2ccc([C@H](C)Nc3ncnc(N4C(=O)OC[C@@H]4C(C)C)n3)nc2)ccc1F] | 2.47 |
| 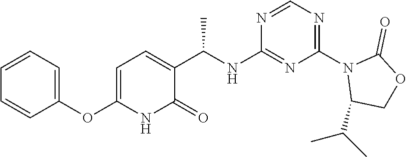 [SMILES: CC(C)[C@H]1COC(=O)N1c1ncnc(N[C@@H](C)c2ccc(Oc3ccccc3)[nH]c2=O)n1] | 2.48 |
| 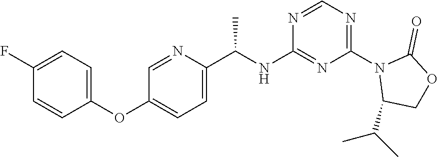 [SMILES: CC(C)[C@H]1COC(=O)N1c1ncnc(N[C@@H](C)c2ccc(Oc3ccc(F)cc3)cn2)n1] | 2.49 |
| 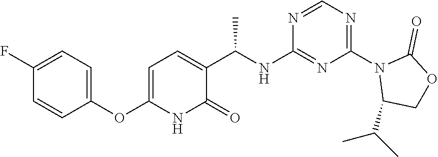 [SMILES: CC(C)[C@H]1COC(=O)N1c1ncnc(N[C@@H](C)c2ccc(Oc3ccc(F)cc3)[nH]c2=O)n1] | 2.50 |
| 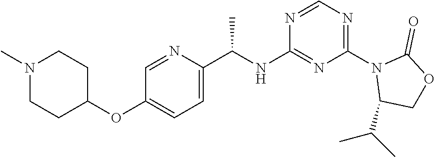 [SMILES: CC(C)[C@H]1COC(=O)N1c1ncnc(N[C@@H](C)c2ccc(OC3CCN(C)CC3)cn2)n1] | 2.51 |
| 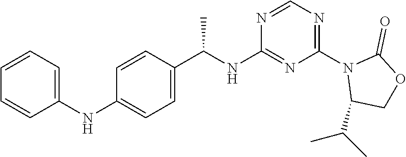 [SMILES: CC(C)[C@H]1COC(=O)N1c1ncnc(N[C@@H](C)c2ccc(Nc3ccccc3)cc2)n1] | 2.52 |
| 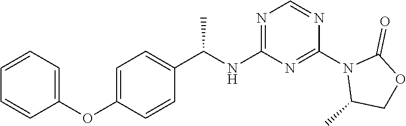 [SMILES: C[C@H](Nc1ncnc(N2C(=O)OC[C@@H]2C)n1)c1ccc(Oc2ccccc2)cc1] | 2.53 |
| 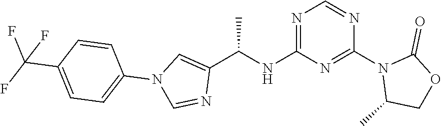 [SMILES: C[C@H](Nc1ncnc(N2C(=O)OC[C@@H]2C)n1)c1cn(-c2ccc(C(F)(F)F)cc2)cn1] | 2.54 |
| 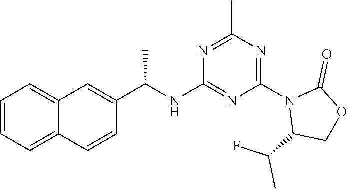 [SMILES: Cc1nc(N[C@@H](C)c2ccc3ccccc3c2)nc(N2C(=O)OCC2[C@H](C)F)n1] | 2.55 |
| 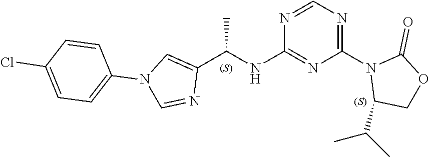 | 2.56 |
| 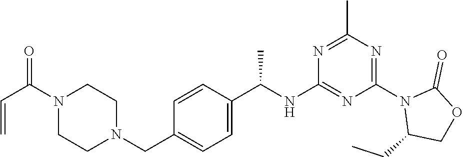 [SMILES: C=CC(=O)N1CCN(Cc2ccc([C@H](C)Nc3nc(C)nc(N4C(=O)OC[C@@H]4CC)n3)cc2)CC1] | 2.57 |
| 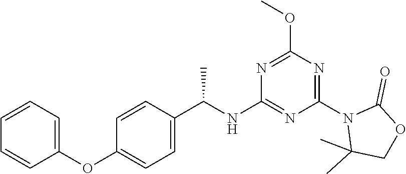 [SMILES: COc1nc(N[C@@H](C)c2ccc(Oc3ccccc3)cc2)nc(N2C(=O)OCC2(C)C)n1] | 2.58 |
| 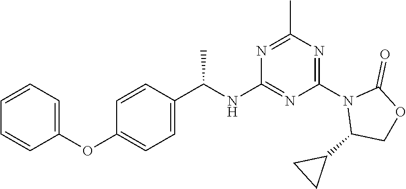 [SMILES: Cc1nc(N[C@@H](C)c2ccc(Oc3ccccc3)cc2)nc(N2C(=O)OC[C@@H]2C2CC2)n1] | 2.59 |
| 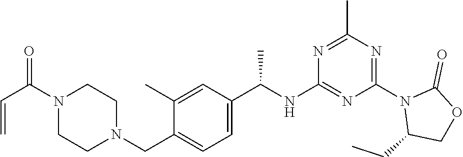 [SMILES: C=CC(=O)N1CCN(Cc2ccc([C@H](C)Nc3nc(C)nc(N4C(=O)OC[C@@H]4CC)n3)cc2C)CC1] | 2.60 |
| 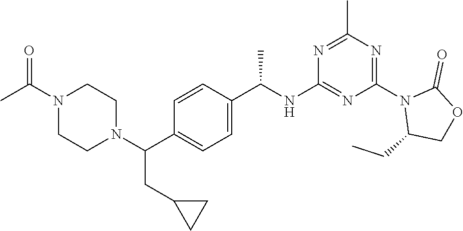 [SMILES: CC[C@H]1COC(=O)N1c1nc(C)nc(N[C@@H](C)c2ccc(C(CC3CC3)N3CCN(C(C)=O)CC3)cc2)n1] | 2.61 |
| 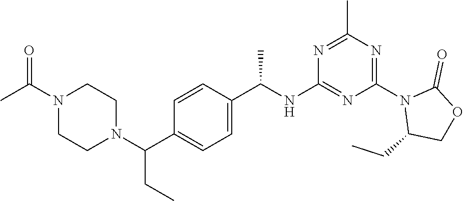 [SMILES: CCC(c1ccc([C@H](C)Nc2nc(C)nc(N3C(=O)OC[C@@H]3CC)n2)cc1)N1CCN(C(C)=O)CC1] | 2.62 |
| 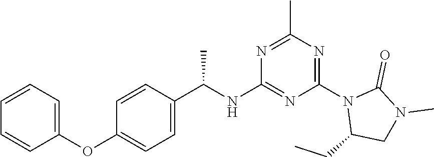 [SMILES: CC[C@H]1CN(C)C(=O)N1c1nc(C)nc(N[C@@H](C)c2ccc(Oc3ccccc3)cc2)n1] | 2.63 |
| 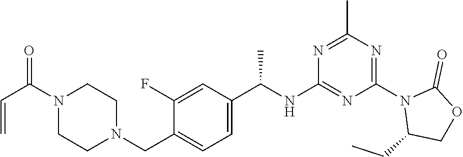 [SMILES: C=CC(=O)N1CCN(Cc2ccc([C@H](C)Nc3nc(C)nc(N4C(=O)OC[C@@H]4CC)n3)cc2F)CC1] | 2.64 |
| 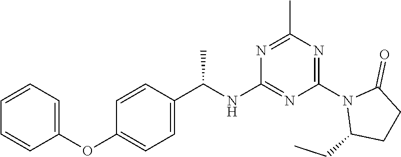 [SMILES: CC[C@H]1CCC(=O)N1c1nc(C)nc(N[C@@H](C)c2ccc(Oc3ccccc3)cc2)n1] | 2.65 |
| 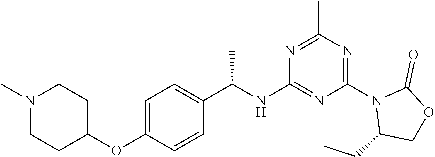 [SMILES: CC[C@H]1COC(=O)N1c1nc(C)nc(N[C@@H](C)c2ccc(OC3CCN(C)CC3)cc2)n1] | 2.66 |
| 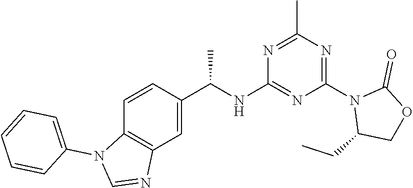 [SMILES: CC[C@H]1COC(=O)N1c1nc(C)nc(N[C@@H](C)c2ccc3c(c2)ncn3-c2ccccc2)n1] | 2.67 |
| 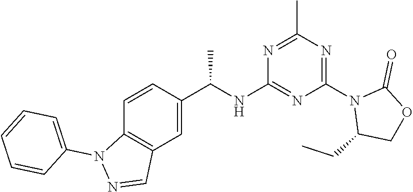 [SMILES: CC[C@H]1COC(=O)N1c1nc(C)nc(N[C@@H](C)c2ccc3c(cnn3-c3ccccc3)c2)n1] | 2.68 |
| 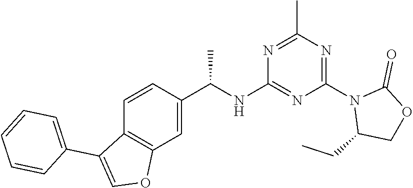 [SMILES: CC[C@H]1COC(=O)N1c1nc(C)nc(N[C@@H](C)c2ccc3c(-c4ccccc4)coc3c2)n1] | 2.69 |
| 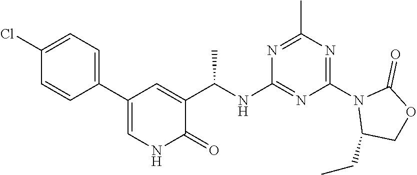 [SMILES: CC[C@H]1COC(=O)N1c1nc(C)nc(N[C@@H](C)c2cc(-c3ccc(Cl)cc3)c[nH]c2=O)n1] | 2.70 |
| 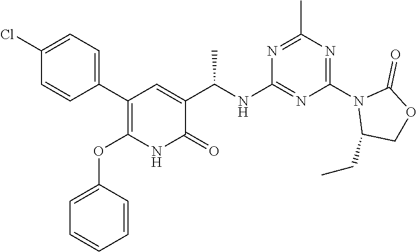 [SMILES: CC[C@H]1COC(=O)N1c1nc(C)nc(N[C@@H](C)c2cc(-c3ccc(Cl)cc3)c(Oc3ccccc3)[nH]c2=O)n1] | 2.71 |
| 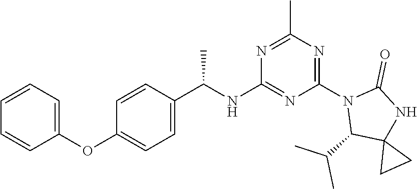 [SMILES: Cc1nc(N[C@@H](C)c2ccc(Oc3ccccc3)cc2)nc(N2C(=O)NC3(CC3)[C@@H]2C(C)C)n1] | 2.72 |
| 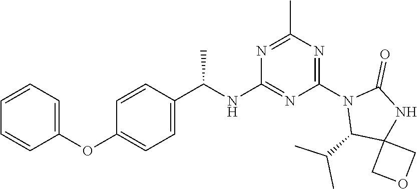 [SMILES: Cc1nc(N[C@@H](C)c2ccc(Oc3ccccc3)cc2)nc(N2C(=O)NC3(COC3)[C@@H]2C(C)C)n1] | 2.73 |
| 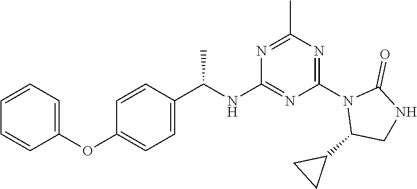 [SMILES: Cc1nc(N[C@@H](C)c2ccc(Oc3ccccc3)cc2)nc(N2C(=O)NC[C@@H]2C2CC2)n1] | 2.74 |
| 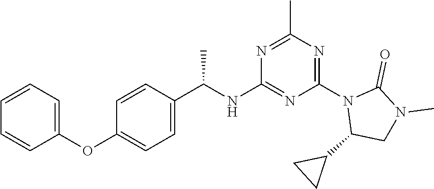 [SMILES: Cc1nc(N[C@@H](C)c2ccc(Oc3ccccc3)cc2)nc(N2C(=O)N(C)C[C@@H]2C2CC2)n1] | 2.75 |
| 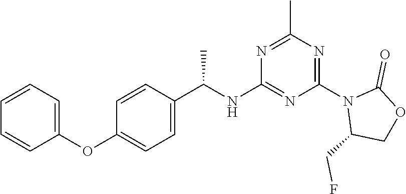 [SMILES: Cc1nc(N[C@@H](C)c2ccc(Oc3ccccc3)cc2)nc(N2C(=O)OC[C@@H]2CF)n1] | 2.76 |
| 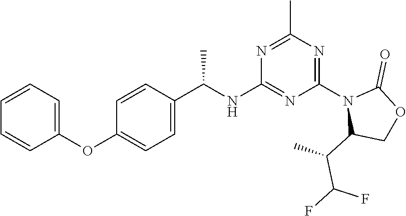 | 2.77 |
| 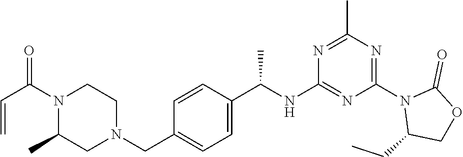 [SMILES: C=CC(=O)N1CCN(Cc2ccc([C@H](C)Nc3nc(C)nc(N4C(=O)OC[C@@H]4CC)n3)cc2)C[C@H]1C] | 2.78 |
| 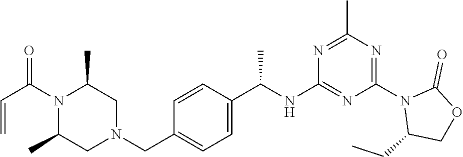 [SMILES: C=CC(=O)N1[C@H](C)CN(Cc2ccc([C@H](C)Nc3nc(C)nc(N4C(=O)OC[C@@H]4CC)n3)cc2)C[C@@H]1C] | 2.79 |
| 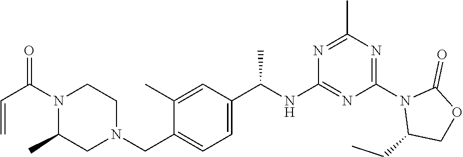 [SMILES: C=CC(=O)N1CCN(Cc2ccc([C@H](C)Nc3nc(C)nc(N4C(=O)OC[C@@H]4CC)n3)cc2C)C[C@H]1C] | 2.80 |
| 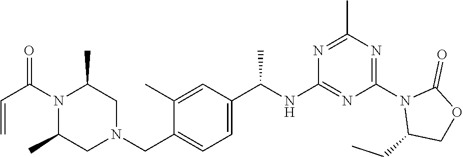 [SMILES: C=CC(=O)N1[C@H](C)CN(Cc2ccc([C@H](C)Nc3nc(C)nc(N4C(=O)OC[C@@H]4CC)n3)cc2C)C[C@@H]1C] | 2.81 |
| 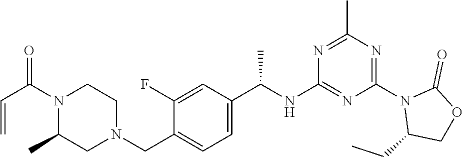 [SMILES: C=CC(=O)N1CCN(Cc2ccc([C@H](C)Nc3nc(C)nc(N4C(=O)OC[C@@H]4CC)n3)cc2F)C[C@H]1C] | 2.82 |
| 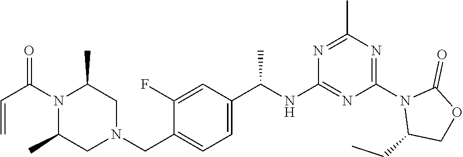 [SMILES: C=CC(=O)N1[C@H](C)CN(Cc2ccc([C@H](C)Nc3nc(C)nc(N4C(=O)OC[C@@H]4CC)n3)cc2F)C[C@@H]1C] | 2.83 |
| 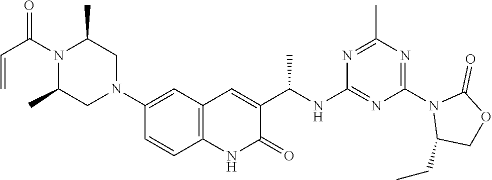 [SMILES: C=CC(=O)N1[C@H](C)CN(c2ccc3[nH]c(=O)c([C@H](C)Nc4nc(C)nc(N5C(=O)OC[C@@H]5CC)n4)cc3c2)C[C@@H]1C] | 2.84 |
| 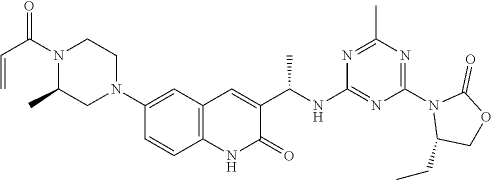 [SMILES: C=CC(=O)N1CCN(c2ccc3[nH]c(=O)c([C@H](C)Nc4nc(C)nc(N5C(=O)OC[C@@H]5CC)n4)cc3c2)C[C@H]1C] | 2.85 |
| 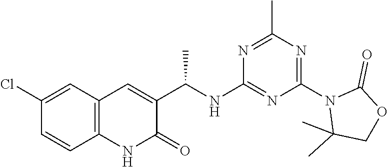 [SMILES: Cc1nc(N[C@@H](C)c2cc3cc(Cl)ccc3[nH]c2=O)nc(N2C(=O)OCC2(C)C)n1] | 2.86 |
| 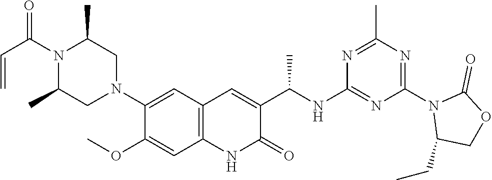 [SMILES: C=CC(=O)N1[C@H](C)CN(c2cc3cc([C@H](C)Nc4nc(C)nc(N5C(=O)OC[C@@H]5CC)n4)c(=O)[nH]c3cc2OC)C[C@@H]1C] | 2.87 |
| 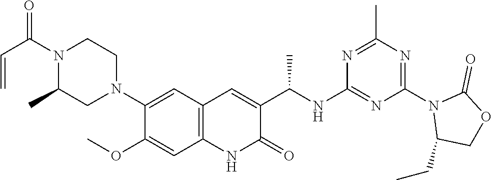 [SMILES: C=CC(=O)N1CCN(c2cc3cc([C@H](C)Nc4nc(C)nc(N5C(=O)OC[C@@H]5CC)n4)c(=O)[nH]c3cc2OC)C[C@H]1C] | 2.88 |
| 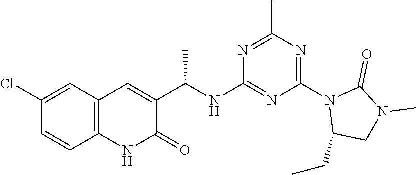 [SMILES: CC[C@H]1CN(C)C(=O)N1c1nc(C)nc(N[C@@H](C)c2cc3cc(Cl)ccc3[nH]c2=O)n1] | 2.89 |
| 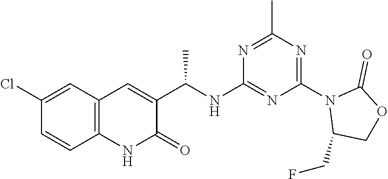 [SMILES: Cc1nc(N[C@@H](C)c2cc3cc(Cl)ccc3[nH]c2=O)nc(N2C(=O)OC[C@@H]2CF)n1] | 2.90 |
| 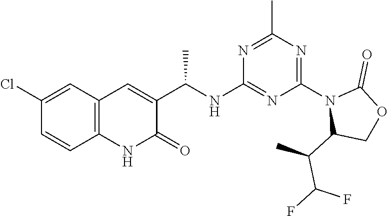 | 2.91 |
| 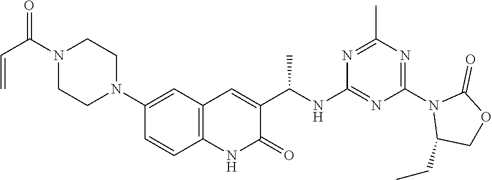 [SMILES: C=CC(=O)N1CCN(c2ccc3[nH]c(=O)c([C@H](C)Nc4nc(C)nc(N5C(=O)OC[C@@H]5CC)n4)cc3c2)CC1] | 2.92 |
| 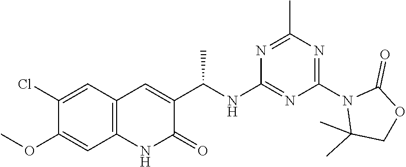 [SMILES: COc1cc2[nH]c(=O)c([C@H](C)Nc3nc(C)nc(N4C(=O)OCC4(C)C)n3)cc2cc1Cl] | 2.93 |
| 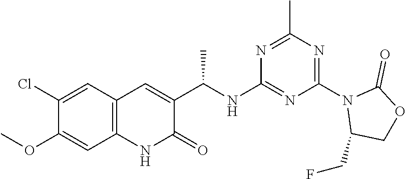 [SMILES: COc1cc2[nH]c(=O)c([C@H](C)Nc3nc(C)nc(N4C(=O)OC[C@@H]4CF)n3)cc2cc1Cl] | 2.94 |
| 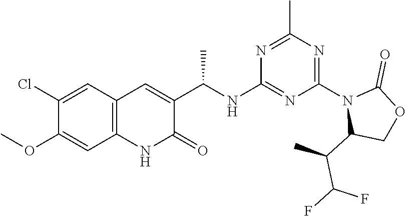 | 2.95 |
| 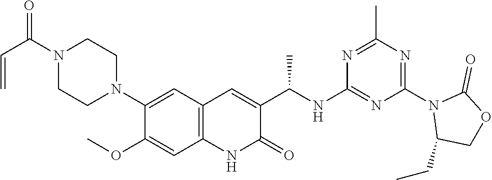 [SMILES: C=CC(=O)N1CCN(c2cc3cc([C@H](C)Nc4nc(C)nc(N5C(=O)OC[C@@H]5CC)n4)c(=O)[nH]c3cc2OC)CC1] | 2.96 |
| 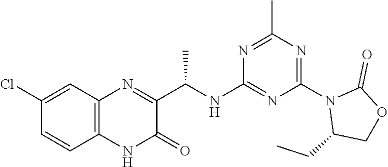 [SMILES: CC[C@H]1COC(=O)N1c1nc(C)nc(N[C@@H](C)c2nc3cc(Cl)ccc3[nH]c2=O)n1] | 2.97 |
| 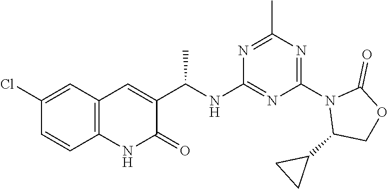 [SMILES: Cc1nc(N[C@@H](C)c2cc3cc(Cl)ccc3[nH]c2=O)nc(N2C(=O)OC[C@@H]2C2CC2)n1] | 2.98 |
| 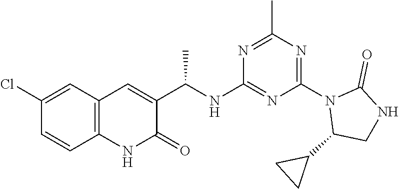 [SMILES: Cc1nc(N[C@@H](C)c2cc3cc(Cl)ccc3[nH]c2=O)nc(N2C(=O)NC[C@@H]2C2CC2)n1] | 2.99 |
| 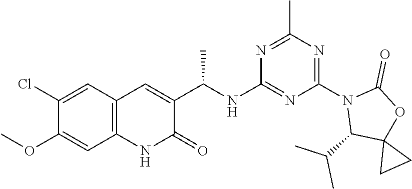 [SMILES: COc1cc2[nH]c(=O)c([C@H](C)Nc3nc(C)nc(N4C(=O)OC5(CC5)[C@@H]4C(C)C)n3)cc2cc1Cl] | 2.100 |
| 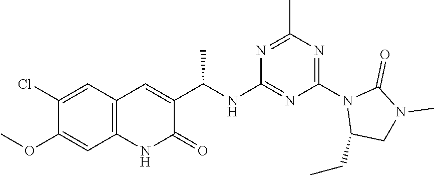 | 2.101 |
| 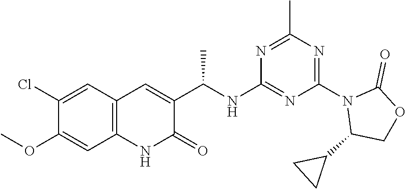 [SMILES: COc1cc2[nH]c(=O)c([C@H](C)Nc3nc(C)nc(N4C(=O)OC[C@@H]4C4CC4)n3)cc2cc1Cl] | 2.102 |
| 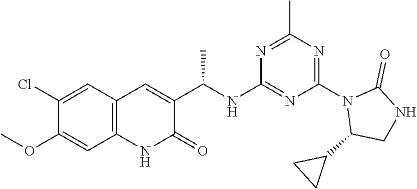 [SMILES: COc1cc2[nH]c(=O)c([C@H](C)Nc3nc(C)nc(N4C(=O)NC[C@@H]4C4CC4)n3)cc2cc1Cl] | 2.103 |
| 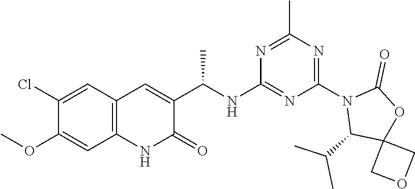 [SMILES: COc1cc2[nH]c(=O)c([C@H](C)Nc3nc(C)nc(N4C(=O)OC5(COC5)[C@@H]4C(C)C)n3)cc2cc1Cl] | 2.104 |
| 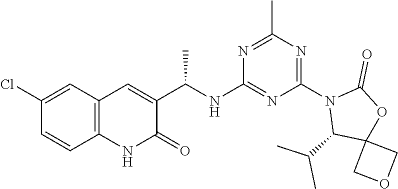 [SMILES: Cc1nc(N[C@@H](C)c2cc3cc(Cl)ccc3[nH]c2=O)nc(N2C(=O)OC3(COC3)[C@@H]2C(C)C)n1] | 2.105 |
| 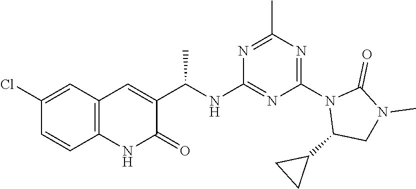 [SMILES: Cc1nc(N[C@@H](C)c2cc3cc(Cl)ccc3[nH]c2=O)nc(N2C(=O)N(C)C[C@@H]2C2CC2)n1] | 2.106 |
| 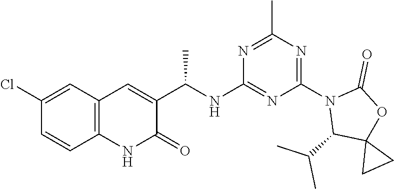 [SMILES: Cc1nc(N[C@@H](C)c2cc3cc(Cl)ccc3[nH]c2=O)nc(N2C(=O)OC3(CC3)[C@@H]2C(C)C)n1] | 2.107 |
| 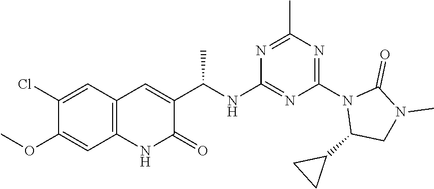 | 2.108 |
| 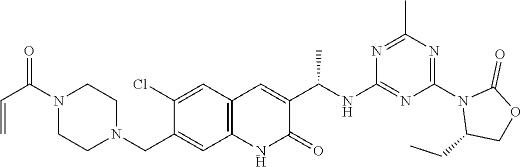 [SMILES: C=CC(=O)N1CCN(Cc2cc3[nH]c(=O)c([C@H](C)Nc4nc(C)nc(N5C(=O)OC[C@@H]5CC)n4)cc3cc2Cl)CC1] | 2.109 |
| 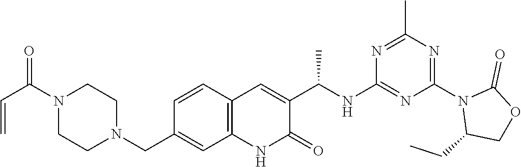 [SMILES: C=CC(=O)N1CCN(Cc2ccc3cc([C@H](C)Nc4nc(C)nc(N5C(=O)OC[C@@H]5CC)n4)c(=O)[nH]c3c2)CC1] | 2.110 |
| 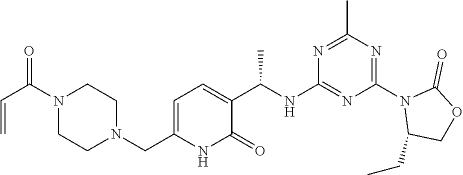 [SMILES: C=CC(=O)N1CCN(Cc2ccc([C@H](C)Nc3nc(C)nc(N4C(=O)OC[C@@H]4CC)n3)c(=O)[nH]2)CC1] | 2.111 |
| 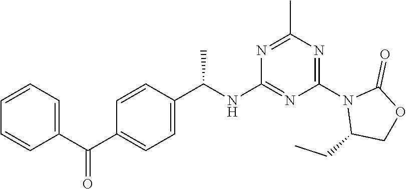 [SMILES: CC[C@H]1COC(=O)N1c1nc(C)nc(N[C@@H](C)c2ccc(C(=O)c3ccccc3)cc2)n1] | 2.112 |
| 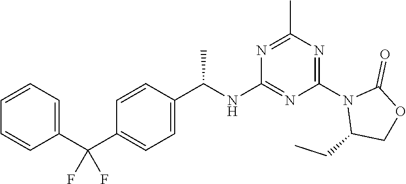 [SMILES: CC[C@H]1COC(=O)N1c1nc(C)nc(N[C@@H](C)c2ccc(C(F)(F)c3ccccc3)cc2)n1] | 2.113 |
| 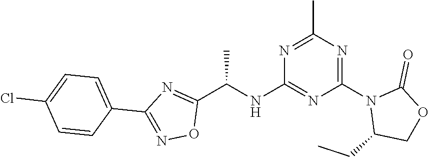 [SMILES: CC[C@H]1COC(=O)N1c1nc(C)nc(N[C@@H](C)c2nc(-c3ccc(Cl)cc3)no2)n1] | 2.114 |
| 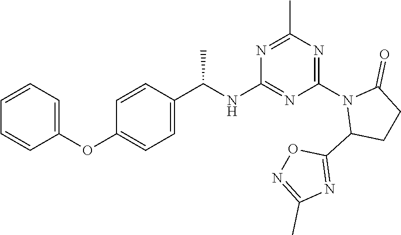 [SMILES: Cc1nc(N[C@@H](C)c2ccc(Oc3ccccc3)cc2)nc(N2C(=O)CCC2c2nc(C)no2)n1] | 2.115 |
| 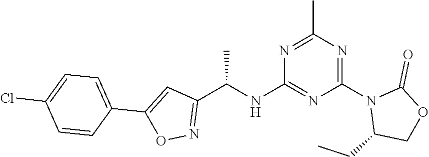 [SMILES: CC[C@H]1COC(=O)N1c1nc(C)nc(N[C@@H](C)c2cc(-c3ccc(Cl)cc3)on2)n1] | 2.116 |
| 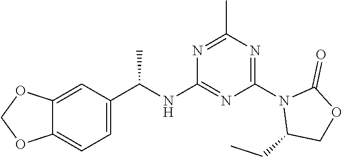 [SMILES: CC[C@H]1COC(=O)N1c1nc(C)nc(N[C@@H](C)c2ccc3c(c2)OCO3)n1] | 2.117 |
| 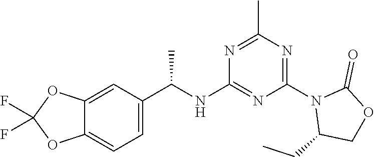 [SMILES: CC[C@H]1COC(=O)N1c1nc(C)nc(N[C@@H](C)c2ccc3c(c2)OC(F)(F)O3)n1] | 2.118 |
| 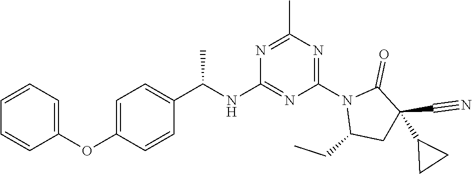 [SMILES: CC[C@H]1C[C@@](C#N)(C2CC2)C(=O)N1c1nc(C)nc(N[C@@H](C)c2ccc(Oc3ccccc3)cc2)n1] | 2.119 |
| 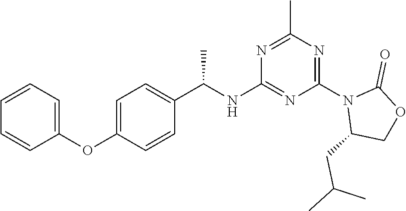 [SMILES: Cc1nc(N[C@@H](C)c2ccc(Oc3ccccc3)cc2)nc(N2C(=O)OC[C@@H]2CC(C)C)n1] | 2.120 |
| 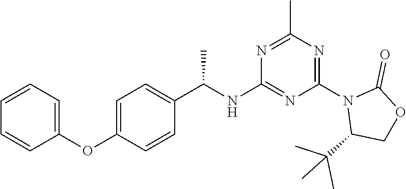 [SMILES: Cc1nc(N[C@@H](C)c2ccc(Oc3ccccc3)cc2)nc(N2C(=O)OC[C@@H]2C(C)(C)C)n1] | 2.121 |
| 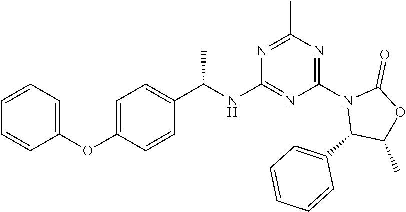 [SMILES: Cc1nc(N[C@@H](C)c2ccc(Oc3ccccc3)cc2)nc(N2C(=O)O[C@H](C)[C@@H]2c2ccccc2)n1] | 2.122 |
| 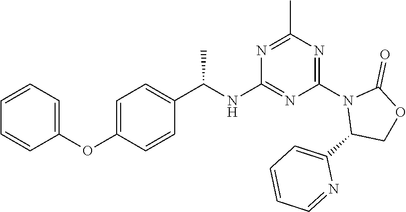 [SMILES: Cc1nc(N[C@@H](C)c2ccc(Oc3ccccc3)cc2)nc(N2C(=O)OC[C@@H]2c2ccccn2)n1] | 2.123 |
| 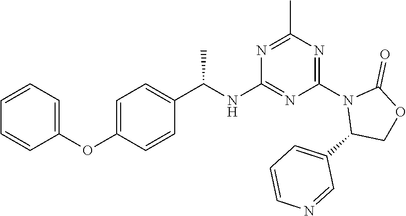 [SMILES: Cc1nc(N[C@@H](C)c2ccc(Oc3ccccc3)cc2)nc(N2C(=O)OC[C@@H]2c2cccnc2)n1] | 2.124 |
| 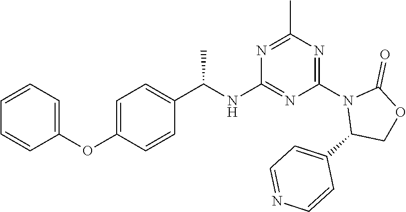 [SMILES: Cc1nc(N[C@@H](C)c2ccc(Oc3ccccc3)cc2)nc(N2C(=O)OC[C@@H]2c2ccncc2)n1] | 2.125 |
| 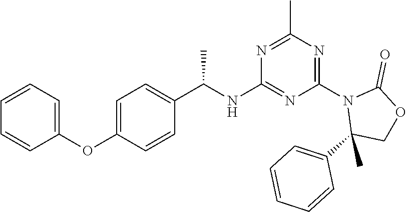 [SMILES: Cc1nc(N[C@@H](C)c2ccc(Oc3ccccc3)cc2)nc(N2C(=O)OC[C@]2(C)c2ccccc2)n1] | 2.126 |
| 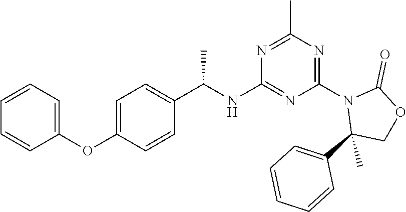 [SMILES: Cc1nc(N[C@@H](C)c2ccc(Oc3ccccc3)cc2)nc(N2C(=O)OC[C@@]2(C)c2ccccc2)n1] | 2.127 |
| 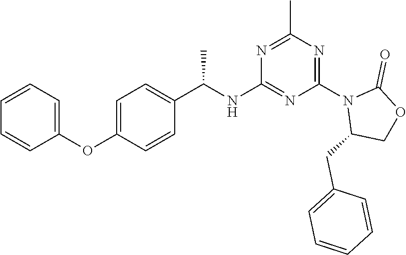 [SMILES: Cc1nc(N[C@@H](C)c2ccc(Oc3ccccc3)cc2)nc(N2C(=O)OC[C@@H]2Cc2ccccc2)n1] | 2.128 |
| 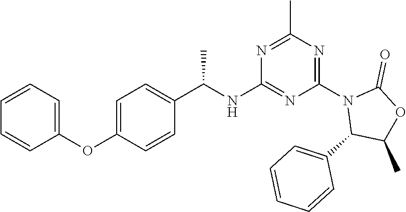 [SMILES: Cc1nc(N[C@@H](C)c2ccc(Oc3ccccc3)cc2)nc(N2C(=O)O[C@@H](C)[C@@H]2c2ccccc2)n1] | 2.129 |
| 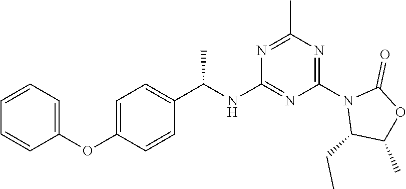 [SMILES: CC[C@H]1[C@@H](C)OC(=O)N1c1nc(C)nc(N[C@@H](C)c2ccc(Oc3ccccc3)cc2)n1] | 2.130 |
| 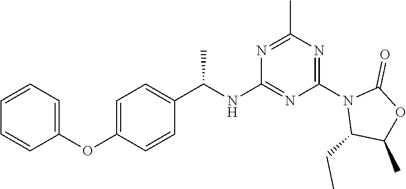 [SMILES: CC[C@H]1[C@H](C)OC(=O)N1c1nc(C)nc(N[C@@H](C)c2ccc(Oc3ccccc3)cc2)n1] | 2.131 |
| 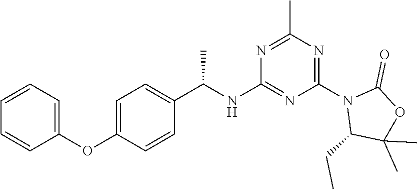 [SMILES: CC[C@@H]1N(c2nc(C)nc(N[C@@H](C)c3ccc(Oc4ccccc4)cc3)n2)C(=O)OC1(C)C] | 2.132 |
| 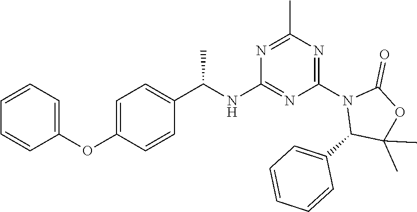 [SMILES: Cc1nc(N[C@@H](C)c2ccc(Oc3ccccc3)cc2)nc(N2C(=O)OC(C)(C)[C@@H]2c2ccccc2)n1] | 2.133 |
| 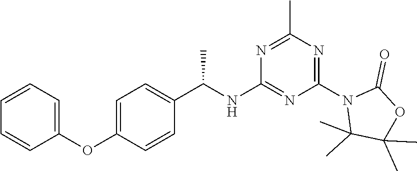 [SMILES: Cc1nc(N[C@@H](C)c2ccc(Oc3ccccc3)cc2)nc(N2C(=O)OC(C)(C)C2(C)C)n1] | 2.134 |
| 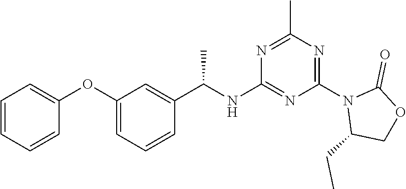 [SMILES: CC[C@H]1COC(=O)N1c1nc(C)nc(N[C@@H](C)c2cccc(Oc3ccccc3)c2)n1] | 2.135 |
| 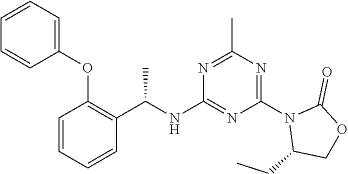 [SMILES: CC[C@H]1COC(=O)N1c1nc(C)nc(N[C@@H](C)c2ccccc2Oc2ccccc2)n1] | 2.136 |
| 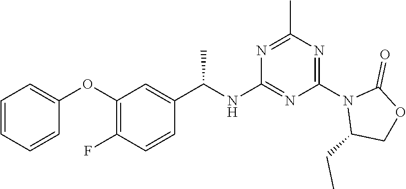 [SMILES: CC[C@H]1COC(=O)N1c1nc(C)nc(N[C@@H](C)c2ccc(F)c(Oc3ccccc3)c2)n1] | 2.137 |
| 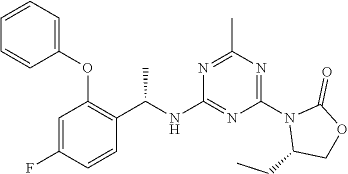 [SMILES: CC[C@H]1COC(=O)N1c1nc(C)nc(N[C@@H](C)c2ccc(F)cc2Oc2ccccc2)n1] | 2.138 |
| 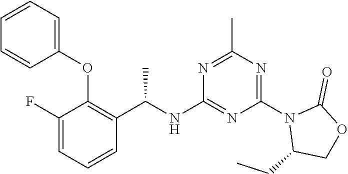 [SMILES: CC[C@H]1COC(=O)N1c1nc(C)nc(N[C@@H](C)c2cccc(F)c2Oc2ccccc2)n1] | 2.139 |
| 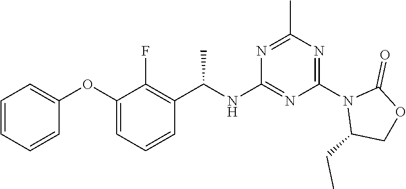 [SMILES: CC[C@H]1COC(=O)N1c1nc(C)nc(N[C@@H](C)c2cccc(Oc3ccccc3)c2F)n1] | 2.140 |
| 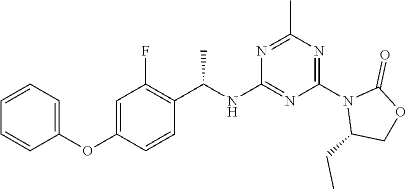 [SMILES: CC[C@H]1COC(=O)N1c1nc(C)nc(N[C@@H](C)c2ccc(Oc3ccccc3)cc2F)n1] | 2.141 |
| 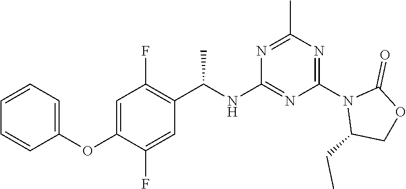 [SMILES: CC[C@H]1COC(=O)N1c1nc(C)nc(N[C@@H](C)c2cc(F)c(Oc3ccccc3)cc2F)n1] | 2.142 |
| 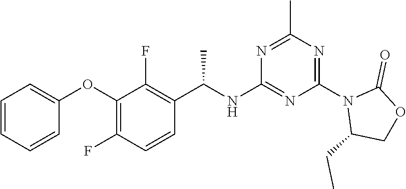 [SMILES: CC[C@H]1COC(=O)N1c1nc(C)nc(N[C@@H](C)c2ccc(F)c(Oc3ccccc3)c2F)n1] | 2.143 |
| 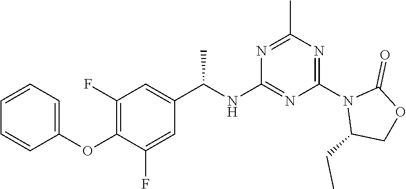 [SMILES: CC[C@H]1COC(=O)N1c1nc(C)nc(N[C@@H](C)c2cc(F)c(Oc3ccccc3)c(F)c2)n1] | 2.144 |
| 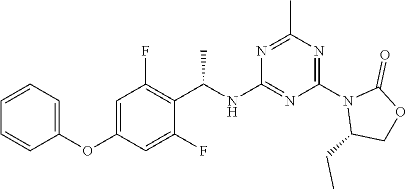 [SMILES: CC[C@H]1COC(=O)N1c1nc(C)nc(N[C@@H](C)c2c(F)cc(Oc3ccccc3)cc2F)n1] | 2.145 |
| 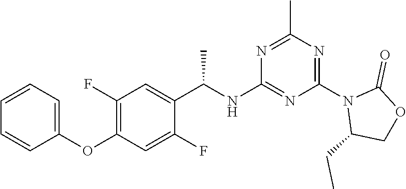 [SMILES: CC[C@H]1COC(=O)N1c1nc(C)nc(N[C@@H](C)c2cc(F)c(Oc3ccccc3)cc2F)n1] | 2.146 |
| 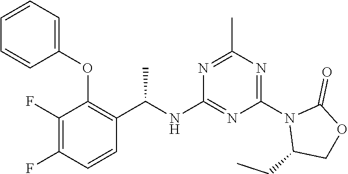 [SMILES: CC[C@H]1COC(=O)N1c1nc(C)nc(N[C@@H](C)c2ccc(F)c(F)c2Oc2ccccc2)n1] | 2.147 |
| 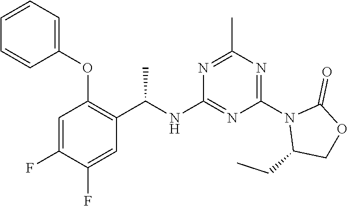 [SMILES: CC[C@H]1COC(=O)N1c1nc(C)nc(N[C@@H](C)c2cc(F)c(F)cc2Oc2ccccc2)n1] | 2.148 |
| 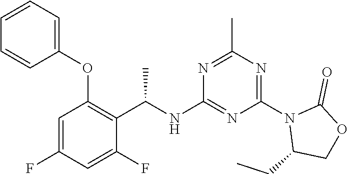 [SMILES: CC[C@H]1COC(=O)N1c1nc(C)nc(N[C@@H](C)c2c(F)cc(F)cc2Oc2ccccc2)n1] | 2.149 |
| 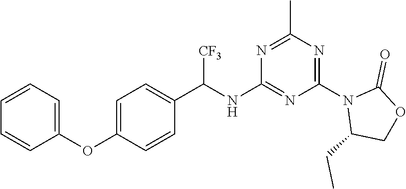 [SMILES: CC[C@H]1COC(=O)N1c1nc(C)nc(NC(c2ccc(Oc3ccccc3)cc2)C(F)(F)F)n1] | 2.150 |
| 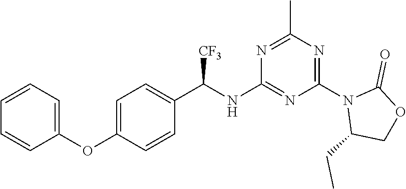 [SMILES: CC[C@H]1COC(=O)N1c1nc(C)nc(N[C@@H](c2ccc(Oc3ccccc3)cc2)C(F)(F)F)n1] | 2.151 |
| 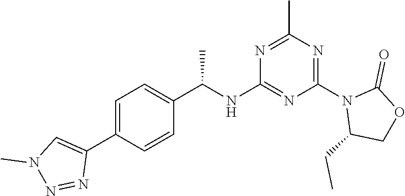 [SMILES: CC[C@H]1COC(=O)N1c1nc(C)nc(N[C@@H](C)c2ccc(-c3cn(C)nn3)cc2)n1] | 2.152 |
| 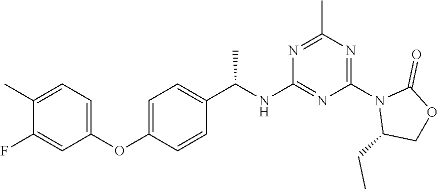 [SMILES: CC[C@H]1COC(=O)N1c1nc(C)nc(N[C@@H](C)c2ccc(Oc3ccc(C)c(F)c3)cc2)n1] | 2.153 |
| 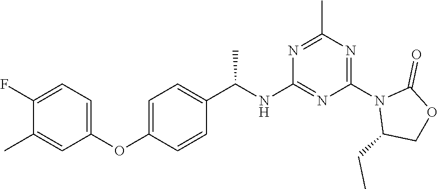 [SMILES: CC[C@H]1COC(=O)N1c1nc(C)nc(N[C@@H](C)c2ccc(Oc3ccc(F)c(C)c3)cc2)n1] | 2.154 |
| 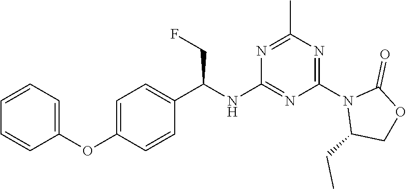 [SMILES: CC[C@H]1COC(=O)N1c1nc(C)nc(N[C@H](CF)c2ccc(Oc3ccccc3)cc2)n1] | 2.155 |
| 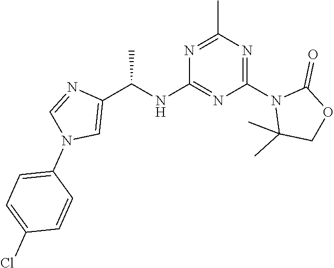 [SMILES: Cc1nc(N[C@@H](C)c2cn(-c3ccc(Cl)cc3)cn2)nc(N2C(=O)OCC2(C)C)n1] | 2.156 |
| 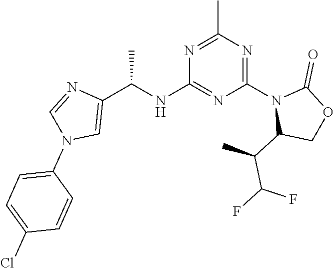 | 2.157 |
| 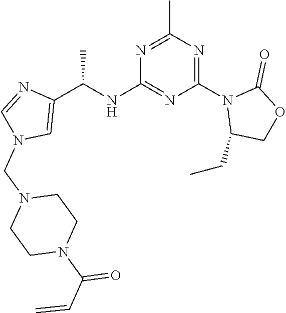 [SMILES: C=CC(=O)N1CCN(Cn2cnc([C@H](C)Nc3nc(C)nc(N4C(=O)OC[C@@H]4CC)n3)c2)CC1] | 2.158 |
| 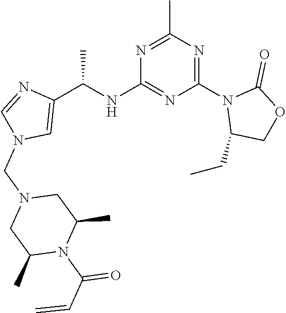 [SMILES: C=CC(=O)N1[C@H](C)CN(Cn2cnc([C@H](C)Nc3nc(C)nc(N4C(=O)OC[C@@H]4CC)n3)c2)C[C@@H]1C] | 2.159 |
| 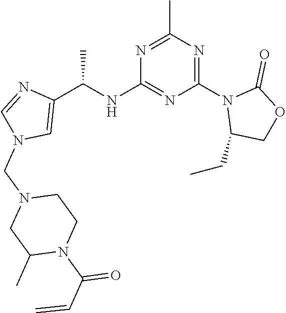 [SMILES: C=CC(=O)N1CCN(Cn2cnc([C@H](C)Nc3nc(C)nc(N4C(=O)OC[C@@H]4CC)n3)c2)CC1C] | 2.160 |
| 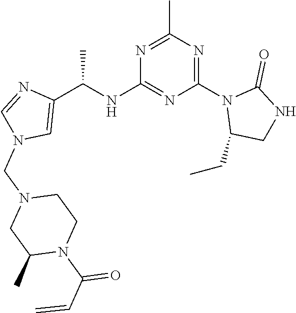 [SMILES: C=CC(=O)N1CCN(Cn2cnc([C@H](C)Nc3nc(C)nc(N4C(=O)NC[C@@H]4CC)n3)c2)C[C@@H]1C] | 2.161 |
| 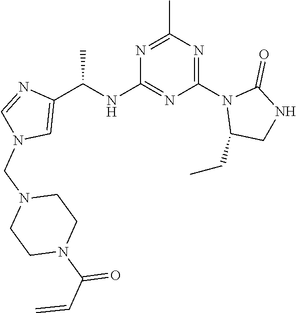 [SMILES: C=CC(=O)N1CCN(Cn2cnc([C@H](C)Nc3nc(C)nc(N4C(=O)NC[C@@H]4CC)n3)c2)CC1] | 2.162 |
| 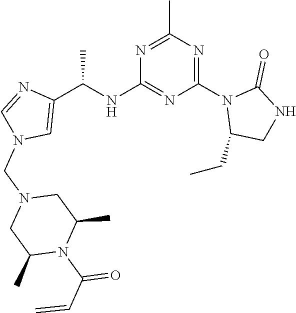 [SMILES: C=CC(=O)N1[C@H](C)CN(Cn2cnc([C@H](C)Nc3nc(C)nc(N4C(=O)NC[C@@H]4CC)n3)c2)C[C@@H]1C] | 2.163 |
| 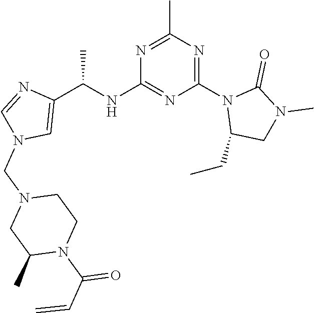 [SMILES: C=CC(=O)N1CCN(Cn2cnc([C@H](C)Nc3nc(C)nc(N4C(=O)N(C)C[C@@H]4CC)n3)c2)C[C@@H]1C] | 2.164 |
| 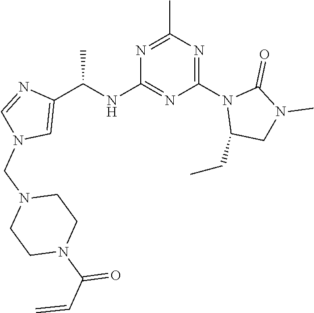 [SMILES: C=CC(=O)N1CCN(Cn2cnc([C@H](C)Nc3nc(C)nc(N4C(=O)N(C)C[C@@H]4CC)n3)c2)CC1] | 2.165 |
| 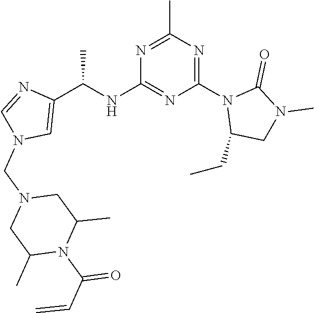 [SMILES: C=CC(=O)N1C(C)CN(Cn2cnc([C@H](C)Nc3nc(C)nc(N4C(=O)N(C)C[C@@H]4CC)n3)c2)CC1C] | 2.166 |
| 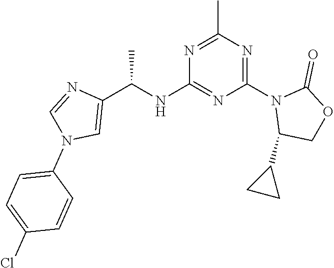 [SMILES: Cc1nc(N[C@@H](C)c2cn(-c3ccc(Cl)cc3)cn2)nc(N2C(=O)OC[C@@H]2C2CC2)n1] | 2.167 |
| 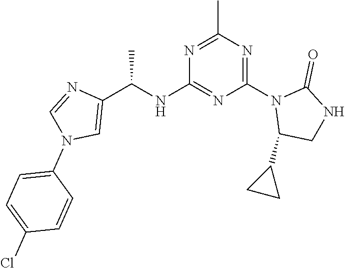 [SMILES: Cc1nc(N[C@@H](C)c2cn(-c3ccc(Cl)cc3)cn2)nc(N2C(=O)NC[C@@H]2C2CC2)n1] | 2.168 |
| 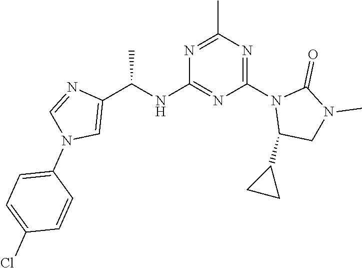 [SMILES: Cc1nc(N[C@@H](C)c2cn(-c3ccc(Cl)cc3)cn2)nc(N2C(=O)N(C)C[C@@H]2C2CC2)n1] | 2.169 |
| 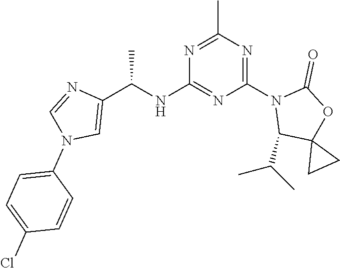 [SMILES: Cc1nc(N[C@@H](C)c2cn(-c3ccc(Cl)cc3)cn2)nc(N2C(=O)OC3(CC3)[C@@H]2C(C)C)n1] | 2.170 |
| 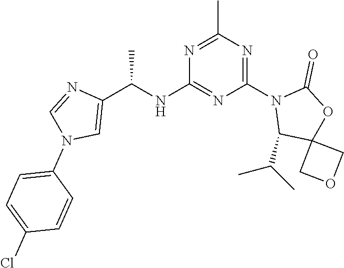 [SMILES: Cc1nc(N[C@@H](C)c2cn(-c3ccc(Cl)cc3)cn2)nc(N2C(=O)OC3(COC3)[C@@H]2C(C)C)n1] | 2.171 |
| 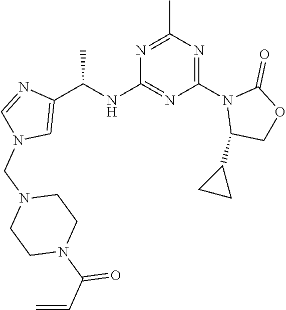 [SMILES: C=CC(=O)N1CCN(Cn2cnc([C@H](C)Nc3nc(C)nc(N4C(=O)OC[C@@H]4C4CC4)n3)c2)CC1] | 2.172 |
| 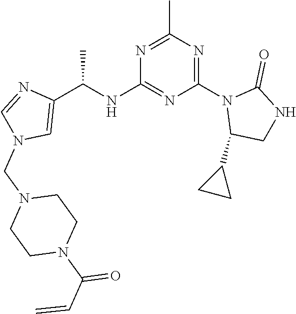 [SMILES: C=CC(=O)N1CCN(Cn2cnc([C@H](C)Nc3nc(C)nc(N4C(=O)NC[C@@H]4C4CC4)n3)c2)CC1] | 2.173 |
| 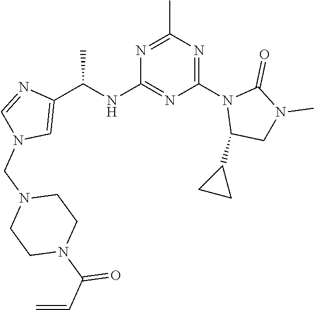 [SMILES: C=CC(=O)N1CCN(Cn2cnc([C@H](C)Nc3nc(C)nc(N4C(=O)N(C)C[C@@H]4C4CC4)n3)c2)CC1] | 2.174 |
| 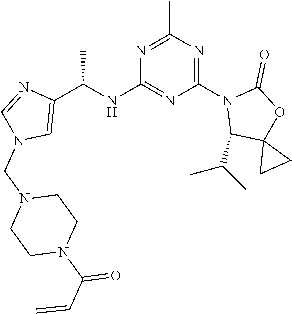 [SMILES: C=CC(=O)N1CCN(Cn2cnc([C@H](C)Nc3nc(C)nc(N4C(=O)OC5(CC5)[C@@H]4C(C)C)n3)c2)CC1] | 2.175 |
| 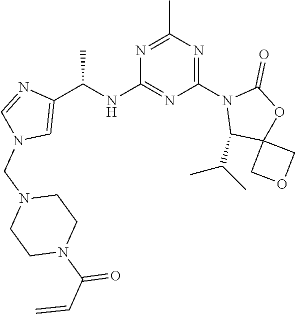 [SMILES: C=CC(=O)N1CCN(Cn2cnc([C@H](C)Nc3nc(C)nc(N4C(=O)OC5(COC5)[C@@H]4C(C)C)n3)c2)CC1] | 2.176 |
| 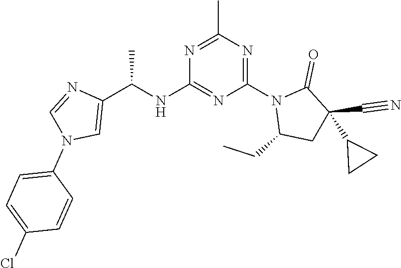 [SMILES: CC[C@H]1C[C@@](C#N)(C2CC2)C(=O)N1c1nc(C)nc(N[C@@H](C)c2cn(-c3ccc(Cl)cc3)cn2)n1] | 2.177 |
| 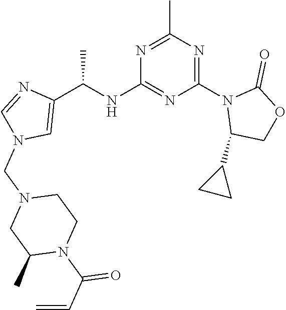 [SMILES: C=CC(=O)N1CCN(Cn2cnc([C@H](C)Nc3nc(C)nc(N4C(=O)OC[C@@H]4C4CC4)n3)c2)C[C@@H]1C] | 2.178 |
| 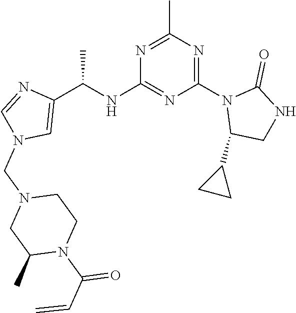 [SMILES: C=CC(=O)N1CCN(Cn2cnc([C@H](C)Nc3nc(C)nc(N4C(=O)NC[C@@H]4C4CC4)n3)c2)C[C@@H]1C] | 2.179 |
| 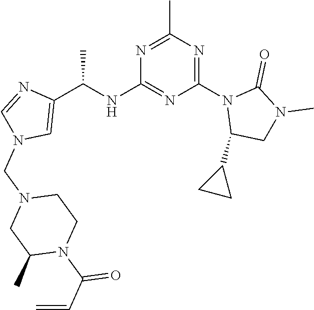 [SMILES: C=CC(=O)N1CCN(Cn2cnc([C@H](C)Nc3nc(C)nc(N4C(=O)N(C)C[C@@H]4C4CC4)n3)c2)C[C@@H]1C] | 2.180 |
| 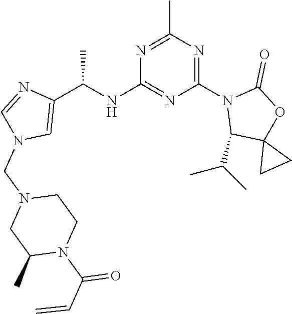 [SMILES: C=CC(=O)N1CCN(Cn2cnc([C@H](C)Nc3nc(C)nc(N4C(=O)OC5(CC5)[C@@H]4C(C)C)n3)c2)C[C@@H]1C] | 2.181 |
| 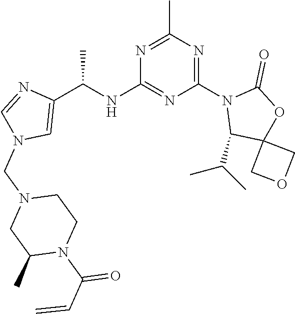 [SMILES: C=CC(=O)N1CCN(Cn2cnc([C@H](C)Nc3nc(C)nc(N4C(=O)OC5(COC5)[C@@H]4C(C)C)n3)c2)C[C@@H]1C] | 2.182 |
| 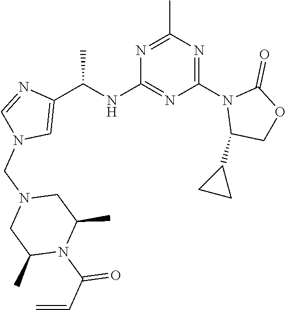 [SMILES: C=CC(=O)N1[C@H](C)CN(Cn2cnc([C@H](C)Nc3nc(C)nc(N4C(=O)OC[C@@H]4C4CC4)n3)c2)C[C@@H]1C] | 2.183 |
| 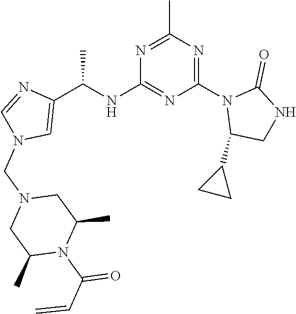 [SMILES: C=CC(=O)N1[C@H](C)CN(Cn2cnc([C@H](C)Nc3nc(C)nc(N4C(=O)NC[C@@H]4C4CC4)n3)c2)C[C@@H]1C] | 2.184 |
| 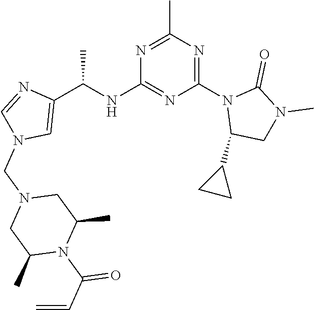 [SMILES: C=CC(=O)N1[C@H](C)CN(Cn2cnc([C@H](C)Nc3nc(C)nc(N4C(=O)N(C)C[C@@H]4C4CC4)n3)c2)C[C@@H]1C] | 2.185 |
| 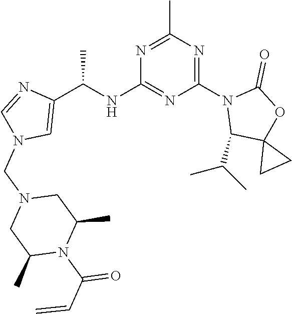 [SMILES: C=CC(=O)N1[C@H](C)CN(Cn2cnc([C@H](C)Nc3nc(C)nc(N4C(=O)OC5(CC5)[C@@H]4C(C)C)n3)c2)C[C@@H]1C] | 2.186 |
| 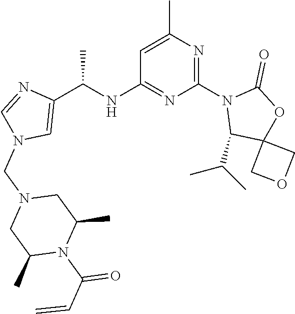 [SMILES: C=CC(=O)N1[C@H](C)CN(Cn2cnc([C@H](C)Nc3cc(C)nc(N4C(=O)OC5(COC5)[C@@H]4C(C)C)n3)c2)C[C@@H]1C] | 2.187 |
| 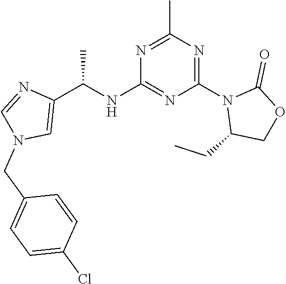 [SMILES: CC[C@H]1COC(=O)N1c1nc(C)nc(N[C@@H](C)c2cn(Cc3ccc(Cl)cc3)cn2)n1] | 2.188 |
| 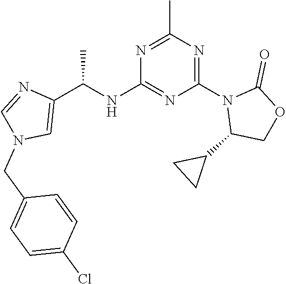 [SMILES: Cc1nc(N[C@@H](C)c2cn(Cc3ccc(Cl)cc3)cn2)nc(N2C(=O)OC[C@@H]2C2CC2)n1] | 2.189 |
| 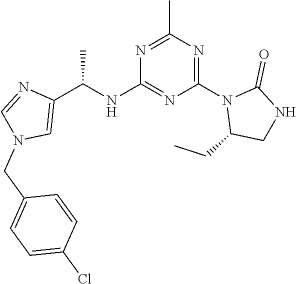 [SMILES: CC[C@H]1CNC(=O)N1c1nc(C)nc(N[C@@H](C)c2cn(Cc3ccc(Cl)cc3)cn2)n1] | 2.190 |
| 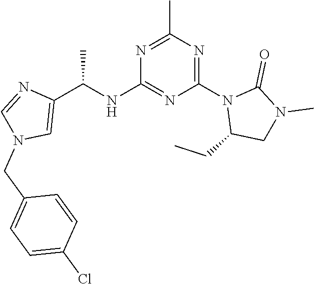 [SMILES: CC[C@H]1CN(C)C(=O)N1c1nc(C)nc(N[C@@H](C)c2cn(Cc3ccc(Cl)cc3)cn2)n1] | 2.191 |
| 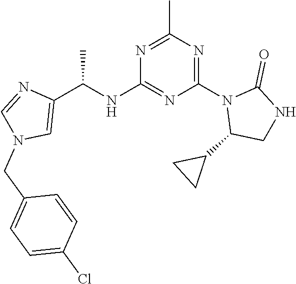 [SMILES: Cc1nc(N[C@@H](C)c2cn(Cc3ccc(Cl)cc3)cn2)nc(N2C(=O)NC[C@@H]2C2CC2)n1] | 2.192 |
| 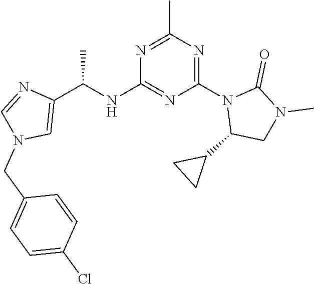 [SMILES: Cc1nc(N[C@@H](C)c2cn(Cc3ccc(Cl)cc3)cn2)nc(N2C(=O)N(C)C[C@@H]2C2CC2)n1] | 2.193 |
| 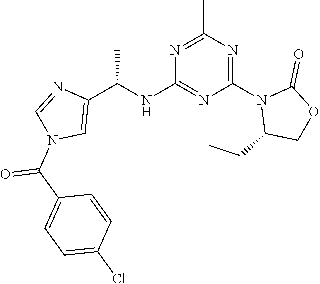 [SMILES: CC[C@H]1COC(=O)N1c1nc(C)nc(N[C@@H](C)c2cn(C(=O)c3ccc(Cl)cc3)cn2)n1] | 2.194 |
| 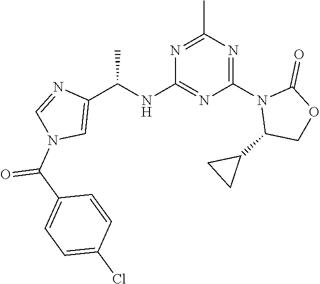 [SMILES: Cc1nc(N[C@@H](C)c2cn(C(=O)c3ccc(Cl)cc3)cn2)nc(N2C(=O)OC[C@@H]2C2CC2)n1] | 2.195 |
| 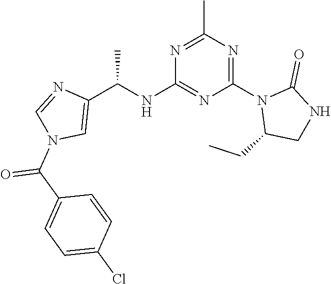 [SMILES: CC[C@H]1CNC(=O)N1c1nc(C)nc(N[C@@H](C)c2cn(C(=O)c3ccc(Cl)cc3)cn2)n1] | 2.196 |
| 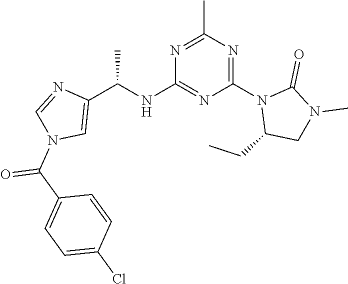 [SMILES: CC[C@H]1CN(C)C(=O)N1c1nc(C)nc(N[C@@H](C)c2cn(C(=O)c3ccc(Cl)cc3)cn2)n1] | 2.197 |
| 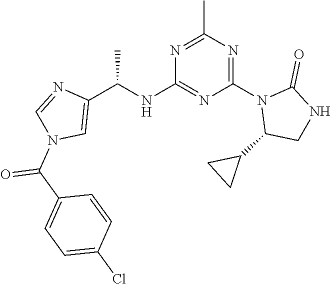 [SMILES: Cc1nc(N[C@@H](C)c2cn(C(=O)c3ccc(Cl)cc3)cn2)nc(N2C(=O)NC[C@@H]2C2CC2)n1] | 2.198 |
| 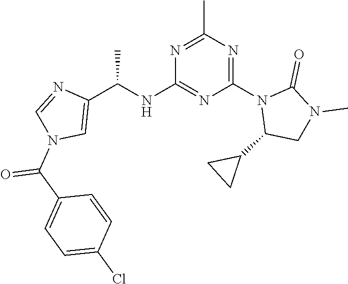 [SMILES: Cc1nc(N[C@@H](C)c2cn(C(=O)c3ccc(Cl)cc3)cn2)nc(N2C(=O)N(C)C[C@@H]2C2CC2)n1] | 2.199 |
| 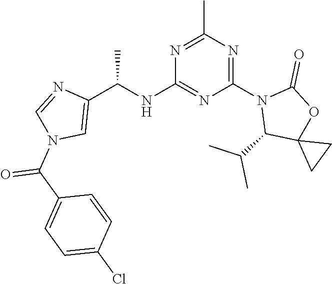 [SMILES: Cc1nc(N[C@@H](C)c2cn(C(=O)c3ccc(Cl)cc3)cn2)nc(N2C(=O)OC3(CC3)[C@@H]2C(C)C)n1] | 2.200 |
| 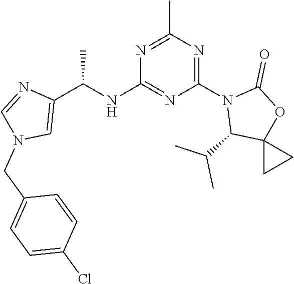 [SMILES: Cc1nc(N[C@@H](C)c2cn(Cc3ccc(Cl)cc3)cn2)nc(N2C(=O)OC3(CC3)[C@@H]2C(C)C)n1] | 2.201 |
| 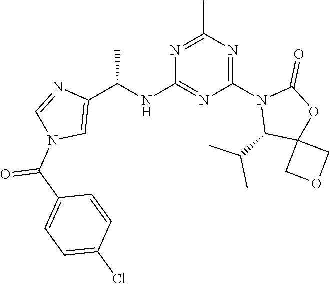 [SMILES: Cc1nc(N[C@@H](C)c2cn(C(=O)c3ccc(Cl)cc3)cn2)nc(N2C(=O)OC3(COC3)[C@@H]2C(C)C)n1] | 2.202 |
| 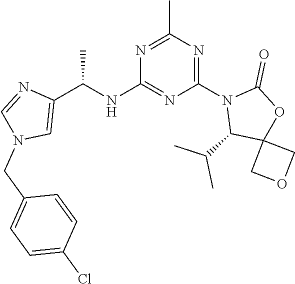 [SMILES: Cc1nc(N[C@@H](C)c2cn(Cc3ccc(Cl)cc3)cn2)nc(N2C(=O)OC3(COC3)[C@@H]2C(C)C)n1] | 2.203 |
| 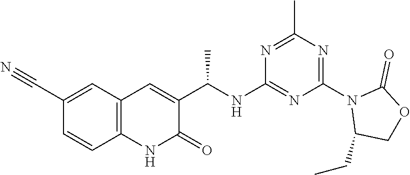 [SMILES: CC[C@H]1COC(=O)N1c1nc(C)nc(N[C@@H](C)c2cc3cc(C#N)ccc3[nH]c2=O)n1] | 2.204 |
| 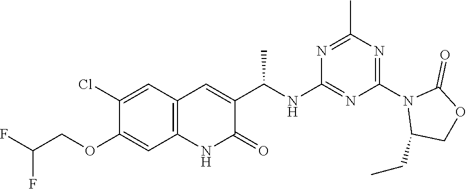 [SMILES: CC[C@H]1COC(=O)N1c1nc(C)nc(N[C@@H](C)c2cc3cc(Cl)c(OCC(F)F)cc3[nH]c2=O)n1] | 2.205 |
| 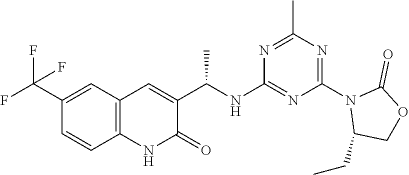 [SMILES: CC[C@H]1COC(=O)N1c1nc(C)nc(N[C@@H](C)c2cc3cc(C(F)(F)F)ccc3[nH]c2=O)n1] | 2.206 |
| 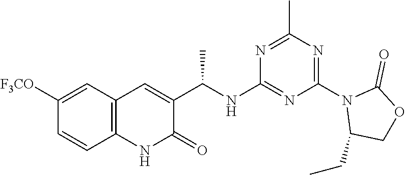 [SMILES: CC[C@H]1COC(=O)N1c1nc(C)nc(N[C@@H](C)c2cc3cc(OC(F)(F)F)ccc3[nH]c2=O)n1] | 2.207 |
| 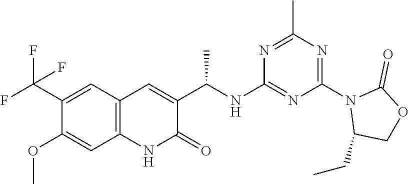 [SMILES: CC[C@H]1COC(=O)N1c1nc(C)nc(N[C@@H](C)c2cc3cc(C(F)(F)F)c(OC)cc3[nH]c2=O)n1] | 2.208 |
| 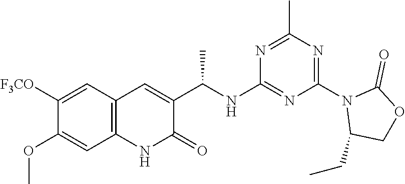 [SMILES: CC[C@H]1COC(=O)N1c1nc(C)nc(N[C@@H](C)c2cc3cc(OC(F)(F)F)c(OC)cc3[nH]c2=O)n1] | 2.209 |
| 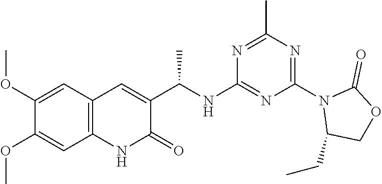 [SMILES: CC[C@H]1COC(=O)N1c1nc(C)nc(N[C@@H](C)c2cc3cc(OC)c(OC)cc3[nH]c2=O)n1] | 2.210 |
| 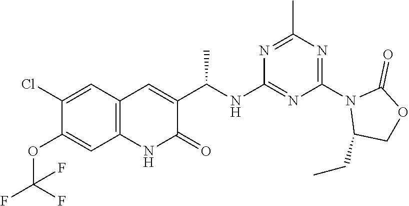 [SMILES: CC[C@H]1COC(=O)N1c1nc(C)nc(N[C@@H](C)c2cc3cc(Cl)c(OC(F)(F)F)cc3[nH]c2=O)n1] | 2.211 |
| 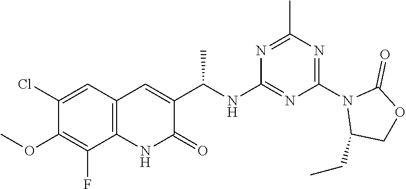 [SMILES: CC[C@H]1COC(=O)N1c1nc(C)nc(N[C@@H](C)c2cc3cc(Cl)c(OC)c(F)c3[nH]c2=O)n1] | 2.212 |
| 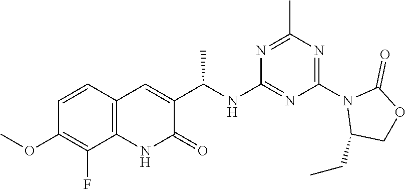 [SMILES: CC[C@H]1COC(=O)N1c1nc(C)nc(N[C@@H](C)c2cc3ccc(OC)c(F)c3[nH]c2=O)n1] | 2.213 |
| 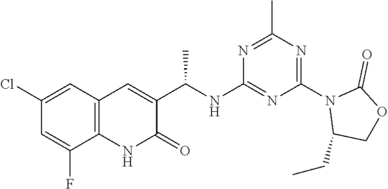 [SMILES: CC[C@H]1COC(=O)N1c1nc(C)nc(N[C@@H](C)c2cc3cc(Cl)cc(F)c3[nH]c2=O)n1] | 2.214 |
| 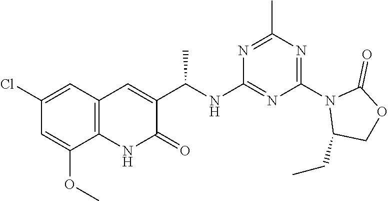 [SMILES: CC[C@H]1COC(=O)N1c1nc(C)nc(N[C@@H](C)c2cc3cc(Cl)cc(OC)c3[nH]c2=O)n1] | 2.215 |
| 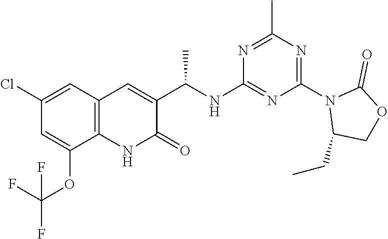 [SMILES: CC[C@H]1COC(=O)N1c1nc(C)nc(N[C@@H](C)c2cc3cc(Cl)cc(OC(F)(F)F)c3[nH]c2=O)n1] | 2.216 |
| 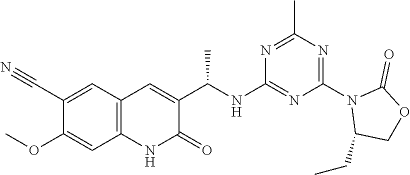 [SMILES: CC[C@H]1COC(=O)N1c1nc(C)nc(N[C@@H](C)c2cc3cc(C#N)c(OC)cc3[nH]c2=O)n1] | 2.217 |
| 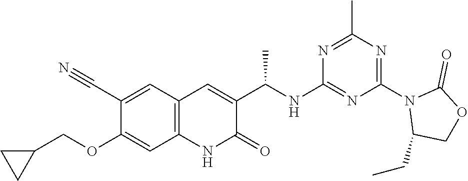 [SMILES: CC[C@H]1COC(=O)N1c1nc(C)nc(N[C@@H](C)c2cc3cc(C#N)c(OCC4CC4)cc3[nH]c2=O)n1] | 2.218 |
| 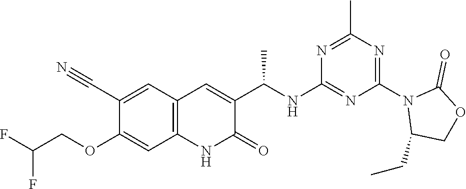 [SMILES: CC[C@H]1COC(=O)N1c1nc(C)nc(N[C@@H](C)c2cc3cc(C#N)c(OCC(F)F)cc3[nH]c2=O)n1] | 2.219 |
| 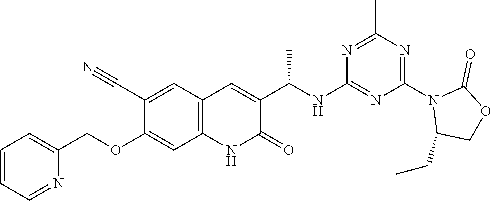 [SMILES: CC[C@H]1COC(=O)N1c1nc(C)nc(N[C@@H](C)c2cc3cc(C#N)c(OCc4ccccn4)cc3[nH]c2=O)n1] | 2.220 |
| 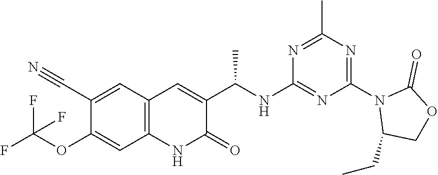 [SMILES: CC[C@H]1COC(=O)N1c1nc(C)nc(N[C@@H](C)c2cc3cc(C#N)c(OC(F)(F)F)cc3[nH]c2=O)n1] | 2.221 |
| 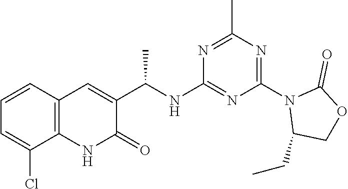 [SMILES: CC[C@H]1COC(=O)N1c1nc(C)nc(N[C@@H](C)c2cc3cccc(Cl)c3[nH]c2=O)n1] | 2.222 |
| 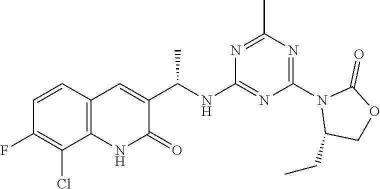 [SMILES: CC[C@H]1COC(=O)N1c1nc(C)nc(N[C@@H](C)c2cc3ccc(F)c(Cl)c3[nH]c2=O)n1] | 2.223 |
| 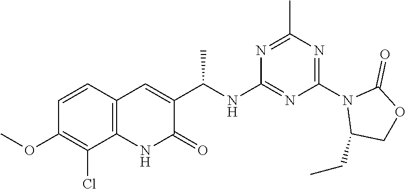 [SMILES: CC[C@H]1COC(=O)N1c1nc(C)nc(N[C@@H](C)c2cc3ccc(OC)c(Cl)c3[nH]c2=O)n1] | 2.224 |
| 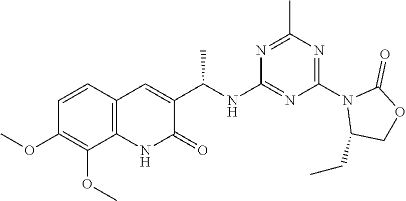 [SMILES: CC[C@H]1COC(=O)N1c1nc(C)nc(N[C@@H](C)c2cc3ccc(OC)c(OC)c3[nH]c2=O)n1] | 2.225 |
| 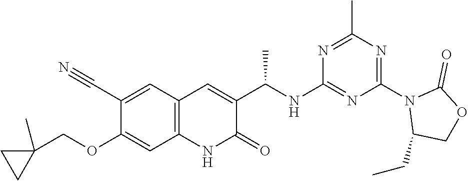 [SMILES: CC[C@H]1COC(=O)N1c1nc(C)nc(N[C@@H](C)c2cc3cc(C#N)c(OCC4(C)CC4)cc3[nH]c2=O)n1] | 2.226 |
| 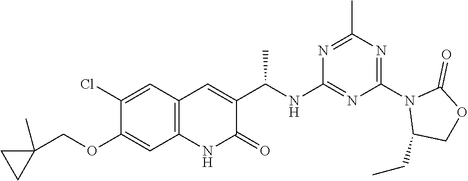 [SMILES: CC[C@H]1COC(=O)N1c1nc(C)nc(N[C@@H](C)c2cc3cc(Cl)c(OCC4(C)CC4)cc3[nH]c2=O)n1] | 2.227 |
| 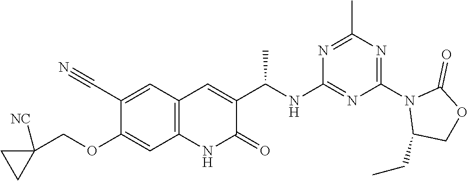 [SMILES: CC[C@H]1COC(=O)N1c1nc(C)nc(N[C@@H](C)c2cc3cc(C#N)c(OCC4(C#N)CC4)cc3[nH]c2=O)n1] | 2.228 |
| 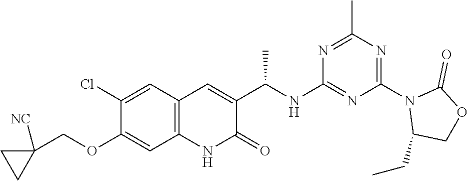 [SMILES: CC[C@H]1COC(=O)N1c1nc(C)nc(N[C@@H](C)c2cc3cc(Cl)c(OCC4(C#N)CC4)cc3[nH]c2=O)n1] | 2.229 |
| 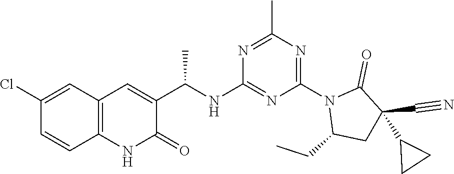 [SMILES: CC[C@H]1C[C@@](C#N)(C2CC2)C(=O)N1c1nc(C)nc(N[C@@H](C)c2cc3cc(Cl)ccc3[nH]c2=O)n1] | 2.230 |
| 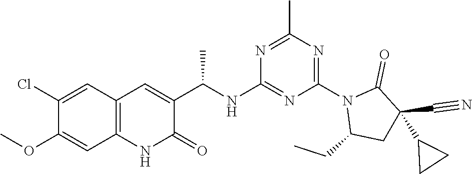 [SMILES: CC[C@H]1C[C@@](C#N)(C2CC2)C(=O)N1c1nc(C)nc(N[C@@H](C)c2cc3cc(Cl)c(OC)cc3[nH]c2=O)n1] | 2.231 |
| 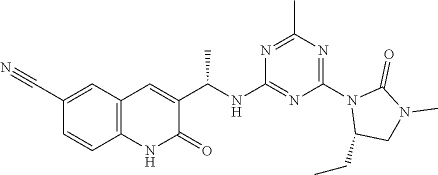 [SMILES: CC[C@H]1CN(C)C(=O)N1c1nc(C)nc(N[C@@H](C)c2cc3cc(C#N)ccc3[nH]c2=O)n1] | 2.232 |
| 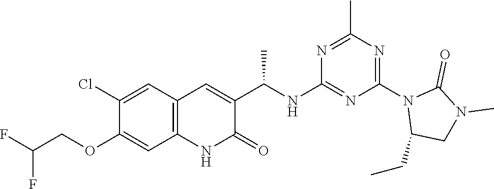 [SMILES: CC[C@H]1CN(C)C(=O)N1c1nc(C)nc(N[C@@H](C)c2cc3cc(Cl)c(OCC(F)F)cc3[nH]c2=O)n1] | 2.233 |
| 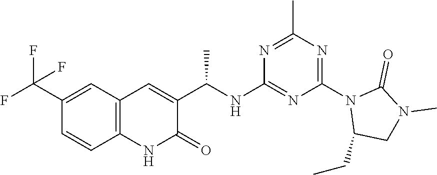 [SMILES: CC[C@H]1CN(C)C(=O)N1c1nc(C)nc(N[C@@H](C)c2cc3cc(C(F)(F)F)ccc3[nH]c2=O)n1] | 2.234 |
| 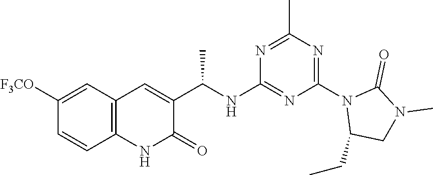 [SMILES: CC[C@H]1CN(C)C(=O)N1c1nc(C)nc(N[C@@H](C)c2cc3cc(OC(F)(F)F)ccc3[nH]c2=O)n1] | 2.235 |
| 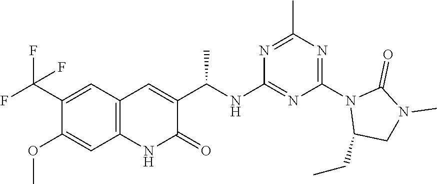 [SMILES: CC[C@H]1CN(C)C(=O)N1c1nc(C)nc(N[C@@H](C)c2cc3cc(C(F)(F)F)c(OC)cc3[nH]c2=O)n1] | 2.236 |
| 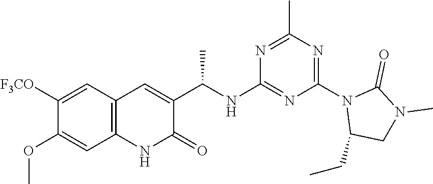 [SMILES: CC[C@H]1CN(C)C(=O)N1c1nc(C)nc(N[C@@H](C)c2cc3cc(OC(F)(F)F)c(OC)cc3[nH]c2=O)n1] | 2.237 |
| 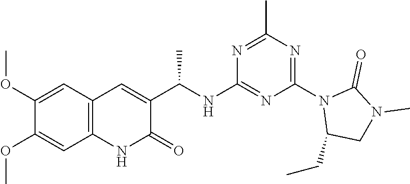 | 2.238 |
| 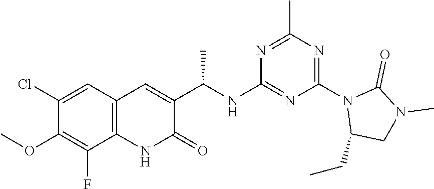 | 2.239 |
| 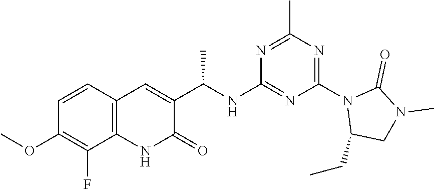 | 2.240 |
| 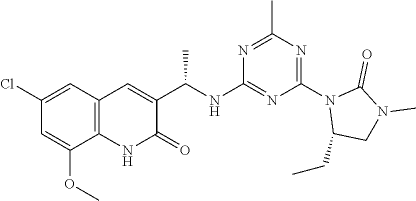 [SMILES: CC[C@H]1CN(C)C(=O)N1c1nc(C)nc(N[C@@H](C)c2cc3cc(Cl)cc(OC)c3[nH]c2=O)n1] | 2.241 |
| 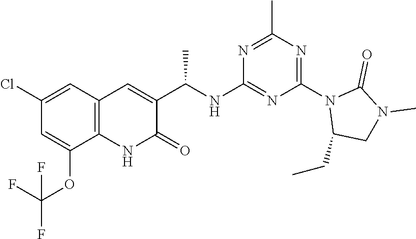 [SMILES: CC[C@H]1CN(C)C(=O)N1c1nc(C)nc(N[C@@H](C)c2cc3cc(Cl)cc(OC(F)(F)F)c3[nH]c2=O)n1] | 2.242 |
| 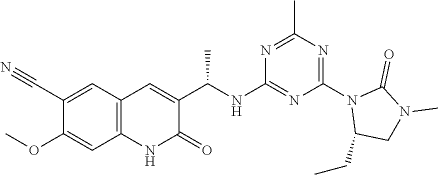 | 2.243 |
| 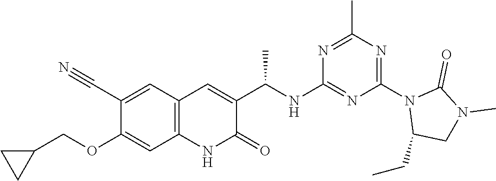 [SMILES: CC[C@H]1CN(C)C(=O)N1c1nc(C)nc(N[C@@H](C)c2cc3cc(C#N)c(OCC4CC4)cc3[nH]c2=O)n1] | 2.244 |
| 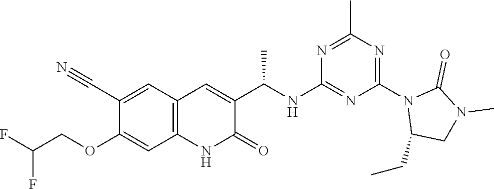 [SMILES: CC[C@H]1CN(C)C(=O)N1c1nc(C)nc(N[C@@H](C)c2cc3cc(C#N)c(OCC(F)F)cc3[nH]c2=O)n1] | 2.245 |
| 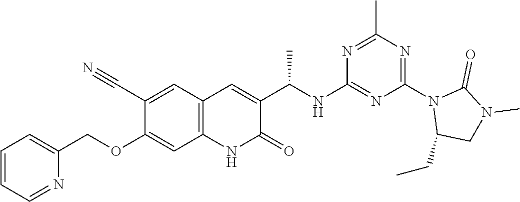 [SMILES: CC[C@H]1CN(C)C(=O)N1c1nc(C)nc(N[C@@H](C)c2cc3cc(C#N)c(OCc4ccccn4)cc3[nH]c2=O)n1] | 2.246 |
| 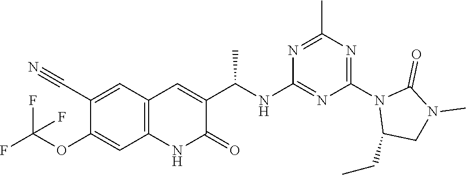 [SMILES: CC[C@H]1CN(C)C(=O)N1c1nc(C)nc(N[C@@H](C)c2cc3cc(C#N)c(OC(F)(F)F)cc3[nH]c2=O)n1] | 2.247 |
| 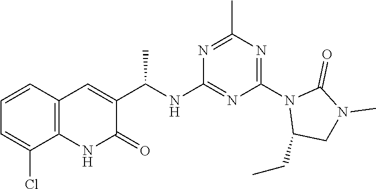 [SMILES: CC[C@H]1CN(C)C(=O)N1c1nc(C)nc(N[C@@H](C)c2cc3cccc(Cl)c3[nH]c2=O)n1] | 2.248 |
| 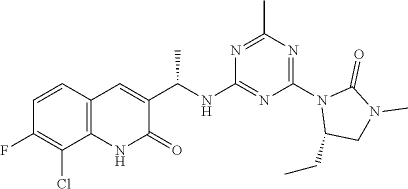 [SMILES: CC[C@H]1CN(C)C(=O)N1c1nc(C)nc(N[C@@H](C)c2cc3ccc(F)c(Cl)c3[nH]c2=O)n1] | 2.249 |
| 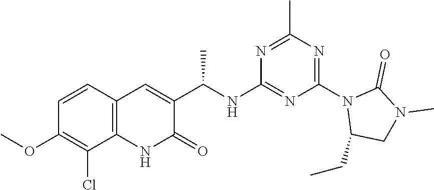 | 2.250 |
| 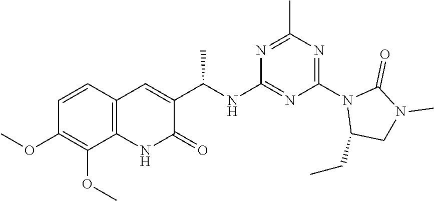 | 2.251 |
| 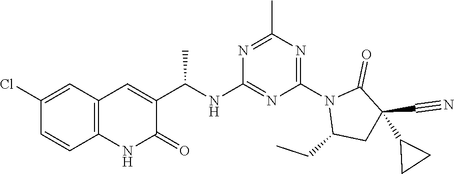 [SMILES: CC[C@H]1C[C@@](C#N)(C2CC2)C(=O)N1c1nc(C)nc(N[C@@H](C)c2cc3cc(Cl)ccc3[nH]c2=O)n1] | 2.252 |
| 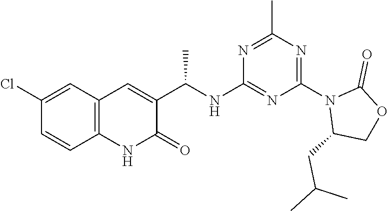 [SMILES: Cc1nc(N[C@@H](C)c2cc3cc(Cl)ccc3[nH]c2=O)nc(N2C(=O)OC[C@@H]2CC(C)C)n1] | 2.253 |
| 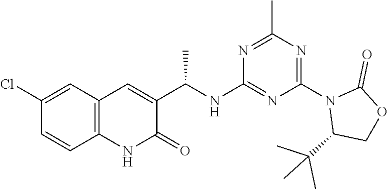 [SMILES: Cc1nc(N[C@@H](C)c2cc3cc(Cl)ccc3[nH]c2=O)nc(N2C(=O)OC[C@@H]2C(C)(C)C)n1] | 2.254 |
| 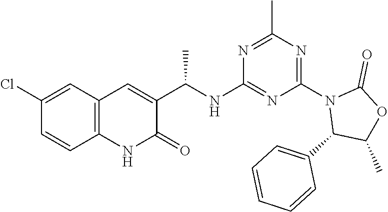 [SMILES: Cc1nc(N[C@@H](C)c2cc3cc(Cl)ccc3[nH]c2=O)nc(N2C(=O)O[C@H](C)[C@@H]2c2ccccc2)n1] | 2.255 |
| 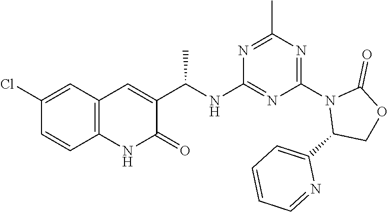 [SMILES: Cc1nc(N[C@@H](C)c2cc3cc(Cl)ccc3[nH]c2=O)nc(N2C(=O)OC[C@@H]2c2ccccn2)n1] | 2.256 |
| 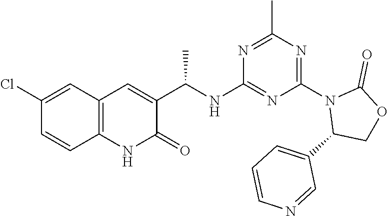 [SMILES: Cc1nc(N[C@@H](C)c2cc3cc(Cl)ccc3[nH]c2=O)nc(N2C(=O)OC[C@@H]2c2cccnc2)n1] | 2.257 |
| 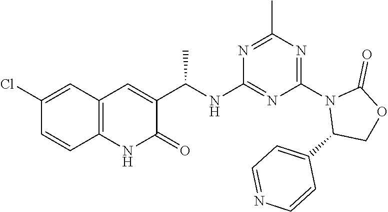 [SMILES: Cc1nc(N[C@@H](C)c2cc3cc(Cl)ccc3[nH]c2=O)nc(N2C(=O)OC[C@@H]2c2ccncc2)n1] | 2.258 |
| 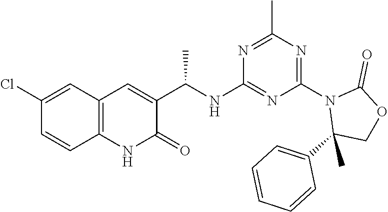 [SMILES: Cc1nc(N[C@@H](C)c2cc3cc(Cl)ccc3[nH]c2=O)nc(N2C(=O)OC[C@]2(C)c2ccccc2)n1] | 2.259 |
| 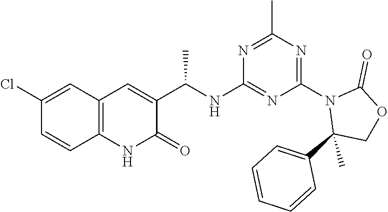 [SMILES: Cc1nc(N[C@@H](C)c2cc3cc(Cl)ccc3[nH]c2=O)nc(N2C(=O)OC[C@@]2(C)c2ccccc2)n1] | 2.260 |
| 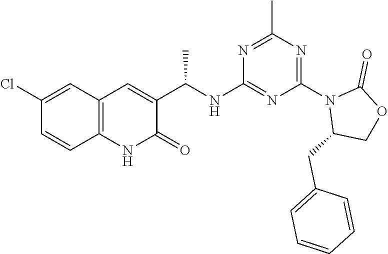 [SMILES: Cc1nc(N[C@@H](C)c2cc3cc(Cl)ccc3[nH]c2=O)nc(N2C(=O)OC[C@@H]2Cc2ccccc2)n1] | 2.261 |
| 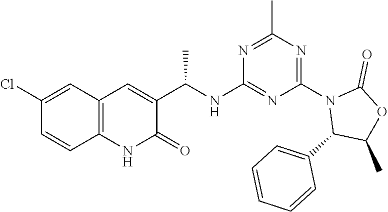 [SMILES: Cc1nc(N[C@@H](C)c2cc3cc(Cl)ccc3[nH]c2=O)nc(N2C(=O)O[C@@H](C)[C@@H]2c2ccccc2)n1] | 2.262 |
| 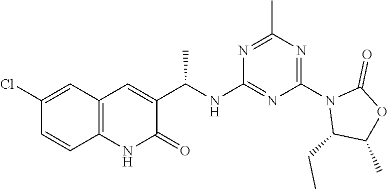 [SMILES: CC[C@H]1[C@@H](C)OC(=O)N1c1nc(C)nc(N[C@@H](C)c2cc3cc(Cl)ccc3[nH]c2=O)n1] | 2.263 |
| 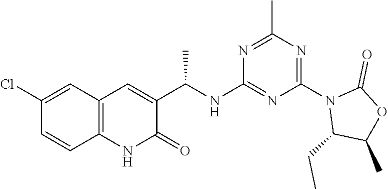 [SMILES: CC[C@H]1[C@H](C)OC(=O)N1c1nc(C)nc(N[C@@H](C)c2cc3cc(Cl)ccc3[nH]c2=O)n1] | 2.264 |
| 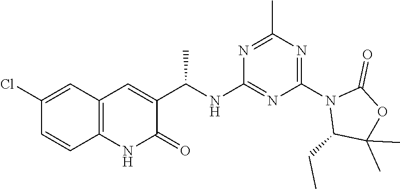 [SMILES: CC[C@@H]1N(c2nc(C)nc(N[C@@H](C)c3cc4cc(Cl)ccc4[nH]c3=O)n2)C(=O)OC1(C)C] | 2.265 |
| 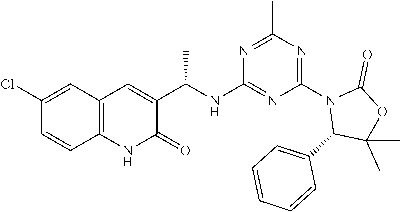 [SMILES: Cc1nc(N[C@@H](C)c2cc3cc(Cl)ccc3[nH]c2=O)nc(N2C(=O)OC(C)(C)[C@@H]2c2ccccc2)n1] | 2.266 |
| 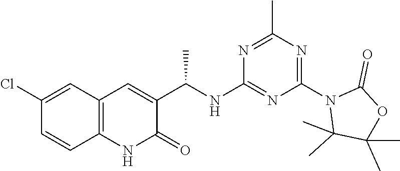 [SMILES: Cc1nc(N[C@@H](C)c2cc3cc(Cl)ccc3[nH]c2=O)nc(N2C(=O)OC(C)(C)C2(C)C)n1] | 2.267 |
| 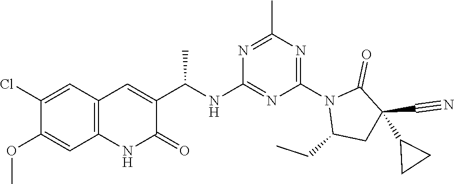 [SMILES: CC[C@H]1C[C@@](C#N)(C2CC2)C(=O)N1c1nc(C)nc(N[C@@H](C)c2cc3cc(Cl)c(OC)cc3[nH]c2=O)n1] | 2.268 |
| 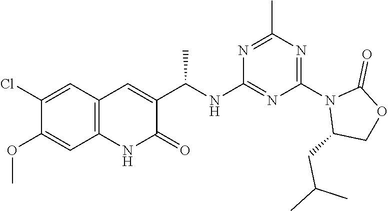 [SMILES: COc1cc2[nH]c(=O)c([C@H](C)Nc3nc(C)nc(N4C(=O)OC[C@@H]4CC(C)C)n3)cc2cc1Cl] | 2.269 |
| 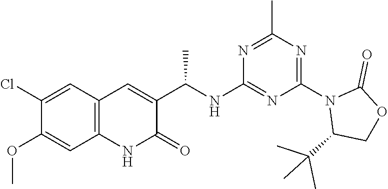 [SMILES: COc1cc2[nH]c(=O)c([C@H](C)Nc3nc(C)nc(N4C(=O)OC[C@@H]4C(C)(C)C)n3)cc2cc1Cl] | 2.270 |
| 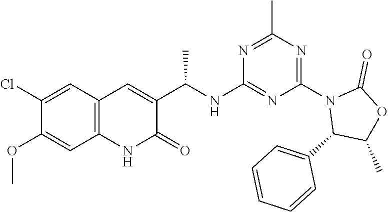 [SMILES: COc1cc2[nH]c(=O)c([C@H](C)Nc3nc(C)nc(N4C(=O)O[C@H](C)[C@@H]4c4ccccc4)n3)cc2cc1Cl] | 2.271 |
| 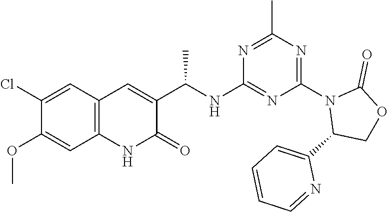 [SMILES: COc1cc2[nH]c(=O)c([C@H](C)Nc3nc(C)nc(N4C(=O)OC[C@@H]4c4ccccn4)n3)cc2cc1Cl] | 2.272 |
| 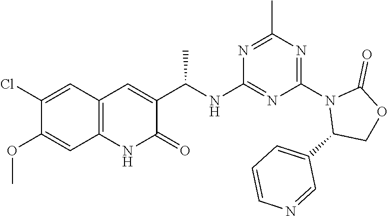 [SMILES: COc1cc2[nH]c(=O)c([C@H](C)Nc3nc(C)nc(N4C(=O)OC[C@@H]4c4cccnc4)n3)cc2cc1Cl] | 2.273 |
| 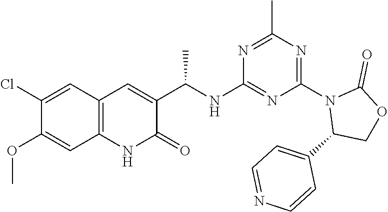 [SMILES: COc1cc2[nH]c(=O)c([C@H](C)Nc3nc(C)nc(N4C(=O)OC[C@@H]4c4ccncc4)n3)cc2cc1Cl] | 2.274 |
| 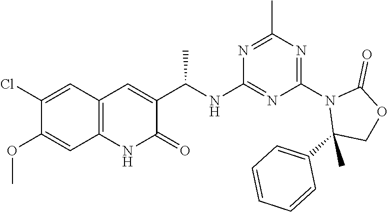 [SMILES: COc1cc2[nH]c(=O)c([C@H](C)Nc3nc(C)nc(N4C(=O)OC[C@]4(C)c4ccccc4)n3)cc2cc1Cl] | 2.275 |
| 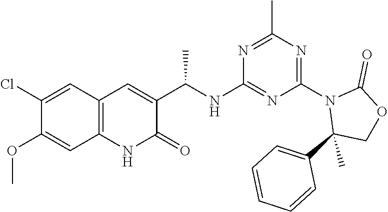 [SMILES: COc1cc2[nH]c(=O)c([C@H](C)Nc3nc(C)nc(N4C(=O)OC[C@@]4(C)c4ccccc4)n3)cc2cc1Cl] | 2.276 |
| 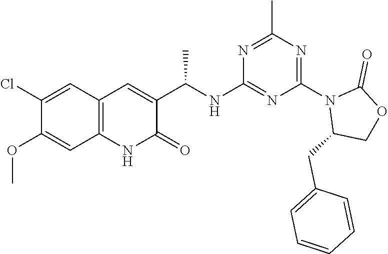 [SMILES: COc1cc2[nH]c(=O)c([C@H](C)Nc3nc(C)nc(N4C(=O)OC[C@@H]4Cc4ccccc4)n3)cc2cc1Cl] | 2.277 |
| 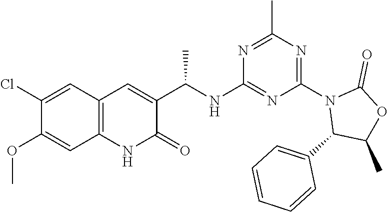 [SMILES: COc1cc2[nH]c(=O)c([C@H](C)Nc3nc(C)nc(N4C(=O)O[C@@H](C)[C@@H]4c4ccccc4)n3)cc2cc1Cl] | 2.278 |
| 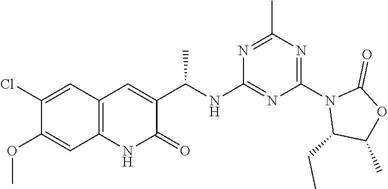 [SMILES: CC[C@H]1[C@@H](C)OC(=O)N1c1nc(C)nc(N[C@@H](C)c2cc3cc(Cl)c(OC)cc3[nH]c2=O)n1] | 2.279 |
| 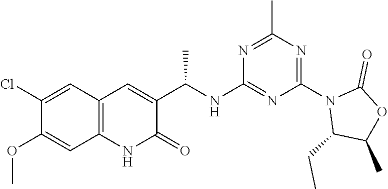 [SMILES: CC[C@H]1[C@H](C)OC(=O)N1c1nc(C)nc(N[C@@H](C)c2cc3cc(Cl)c(OC)cc3[nH]c2=O)n1] | 2.280 |
| 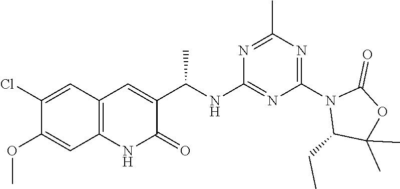 [SMILES: CC[C@@H]1N(c2nc(C)nc(N[C@@H](C)c3cc4cc(Cl)c(OC)cc4[nH]c3=O)n2)C(=O)OC1(C)C] | 2.281 |
| 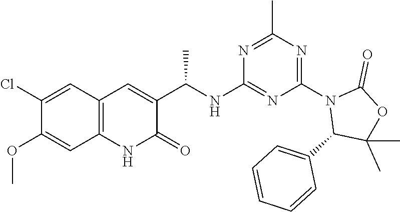 [SMILES: COc1cc2[nH]c(=O)c([C@H](C)Nc3nc(C)nc(N4C(=O)OC(C)(C)[C@@H]4c4ccccc4)n3)cc2cc1Cl] | 2.282 |
| 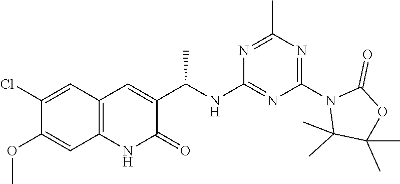 [SMILES: COc1cc2[nH]c(=O)c([C@H](C)Nc3nc(C)nc(N4C(=O)OC(C)(C)C4(C)C)n3)cc2cc1Cl] | 2.283 |
| 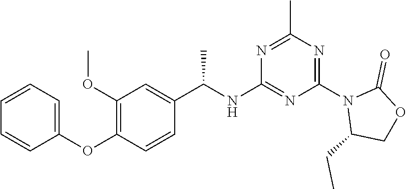 [SMILES: CC[C@H]1COC(=O)N1c1nc(C)nc(N[C@@H](C)c2ccc(Oc3ccccc3)c(OC)c2)n1] | 2.284 |
| 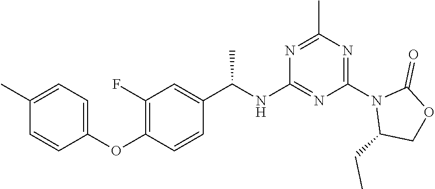 [SMILES: CC[C@H]1COC(=O)N1c1nc(C)nc(N[C@@H](C)c2ccc(Oc3ccc(C)cc3)c(F)c2)n1] | 2.285 |
| 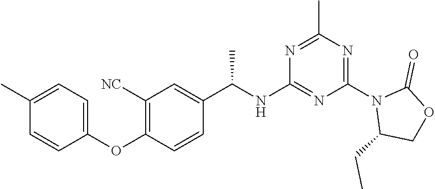 [SMILES: CC[C@H]1COC(=O)N1c1nc(C)nc(N[C@@H](C)c2ccc(Oc3ccc(C)cc3)c(C#N)c2)n1] | 2.286 |
| 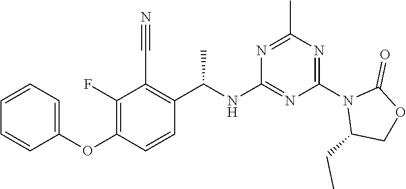 [SMILES: CC[C@H]1COC(=O)N1c1nc(C)nc(N[C@@H](C)c2ccc(Oc3ccccc3)c(F)c2C#N)n1] | 2.287 |
| 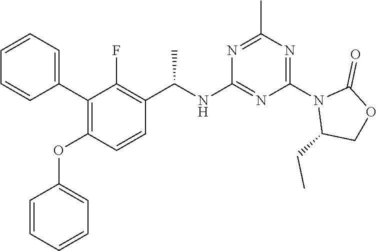 [SMILES: CC[C@H]1COC(=O)N1c1nc(C)nc(N[C@@H](C)c2ccc(Oc3ccccc3)c(-c3ccccc3)c2F)n1] | 2.288 |
| 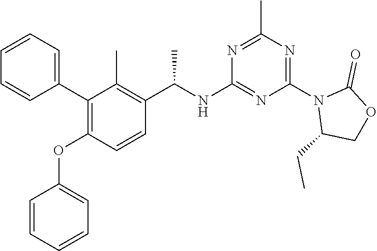 [SMILES: CC[C@H]1COC(=O)N1c1nc(C)nc(N[C@@H](C)c2ccc(Oc3ccccc3)c(-c3ccccc3)c2C)n1] | 2.289 |
| 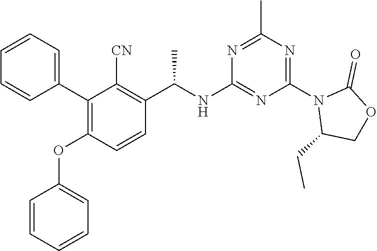 [SMILES: CC[C@H]1COC(=O)N1c1nc(C)nc(N[C@@H](C)c2ccc(Oc3ccccc3)c(-c3ccccc3)c2C#N)n1] | 2.290 |
| 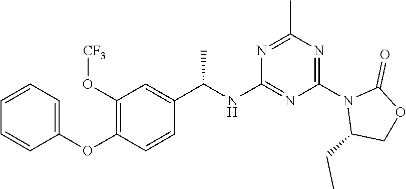 [SMILES: CC[C@H]1COC(=O)N1c1nc(C)nc(N[C@@H](C)c2ccc(Oc3ccccc3)c(OC(F)(F)F)c2)n1] | 2.291 |
| 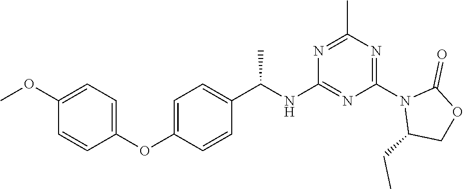 [SMILES: CC[C@H]1COC(=O)N1c1nc(C)nc(N[C@@H](C)c2ccc(Oc3ccc(OC)cc3)cc2)n1] | 2.292 |
| 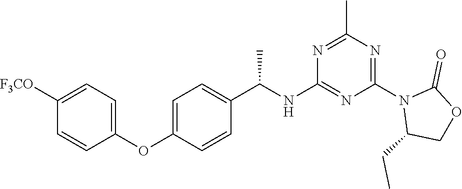 [SMILES: CC[C@H]1COC(=O)N1c1nc(C)nc(N[C@@H](C)c2ccc(Oc3ccc(OC(F)(F)F)cc3)cc2)n1] | 2.293 |
| 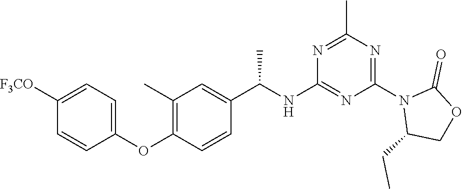 [SMILES: CC[C@H]1COC(=O)N1c1nc(C)nc(N[C@@H](C)c2ccc(Oc3ccc(OC(F)(F)F)cc3)c(C)c2)n1] | 2.294 |
| 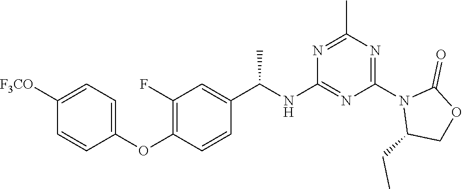 [SMILES: CC[C@H]1COC(=O)N1c1nc(C)nc(N[C@@H](C)c2ccc(Oc3ccc(OC(F)(F)F)cc3)c(F)c2)n1] | 2.295 |
| 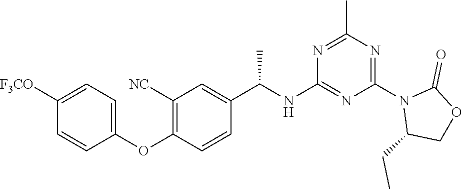 [SMILES: CC[C@H]1COC(=O)N1c1nc(C)nc(N[C@@H](C)c2ccc(Oc3ccc(OC(F)(F)F)cc3)c(C#N)c2)n1] | 2.296 |
| 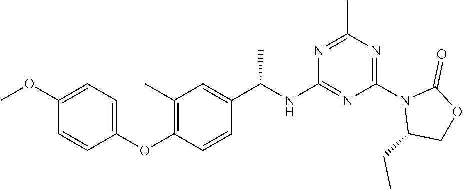 [SMILES: CC[C@H]1COC(=O)N1c1nc(C)nc(N[C@@H](C)c2ccc(Oc3ccc(OC)cc3)c(C)c2)n1] | 2.297 |
| 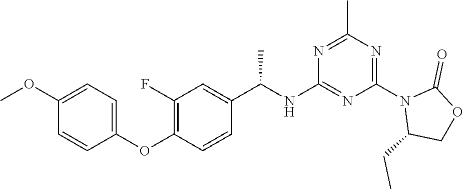 [SMILES: CC[C@H]1COC(=O)N1c1nc(C)nc(N[C@@H](C)c2ccc(Oc3ccc(OC)cc3)c(F)c2)n1] | 2.298 |
| 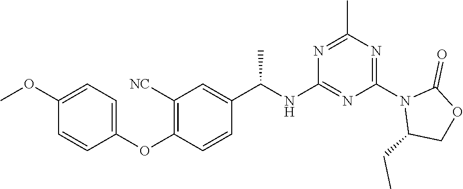 [SMILES: CC[C@H]1COC(=O)N1c1nc(C)nc(N[C@@H](C)c2ccc(Oc3ccc(OC)cc3)c(C#N)c2)n1] | 2.299 |
| 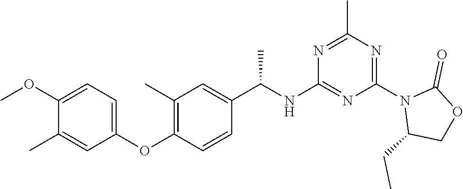 [SMILES: CC[C@H]1COC(=O)N1c1nc(C)nc(N[C@@H](C)c2ccc(Oc3ccc(OC)c(C)c3)c(C)c2)n1] | 2.300 |
| 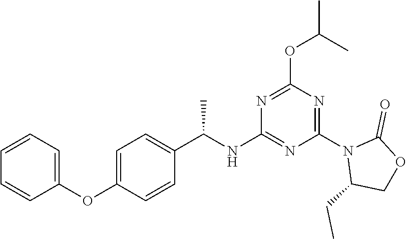 [SMILES: CC[C@H]1COC(=O)N1c1nc(N[C@@H](C)c2ccc(Oc3ccccc3)cc2)nc(OC(C)C)n1] | 2.301 |
| 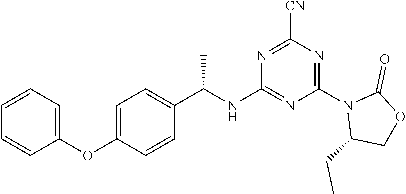 [SMILES: CC[C@H]1COC(=O)N1c1nc(C#N)nc(N[C@@H](C)c2ccc(Oc3ccccc3)cc2)n1] | 2.302 |
| 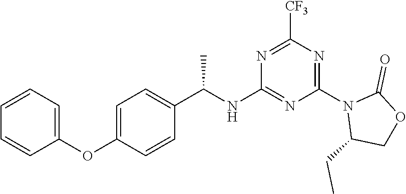 [SMILES: CC[C@H]1COC(=O)N1c1nc(N[C@@H](C)c2ccc(Oc3ccccc3)cc2)nc(C(F)(F)F)n1] | 2.303 |
| 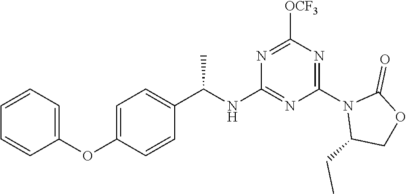 [SMILES: CC[C@H]1COC(=O)N1c1nc(N[C@@H](C)c2ccc(Oc3ccccc3)cc2)nc(OC(F)(F)F)n1] | 2.304 |
| 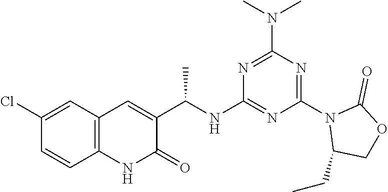 [SMILES: CC[C@H]1COC(=O)N1c1nc(N[C@@H](C)c2cc3cc(Cl)ccc3[nH]c2=O)nc(N(C)C)n1] | 2.305 |
| 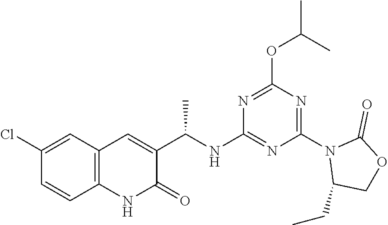 [SMILES: CC[C@H]1COC(=O)N1c1nc(N[C@@H](C)c2cc3cc(Cl)ccc3[nH]c2=O)nc(OC(C)C)n1] | 2.306 |
| 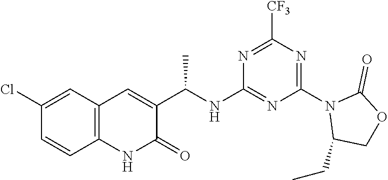 [SMILES: CC[C@H]1COC(=O)N1c1nc(N[C@@H](C)c2cc3cc(Cl)ccc3[nH]c2=O)nc(C(F)(F)F)n1] | 2.307 |
| 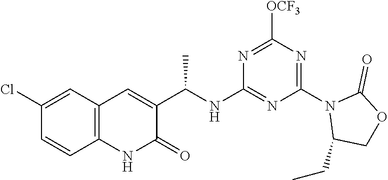 [SMILES: CC[C@H]1COC(=O)N1c1nc(N[C@@H](C)c2cc3cc(Cl)ccc3[nH]c2=O)nc(OC(F)(F)F)n1] | 2.308 |
| 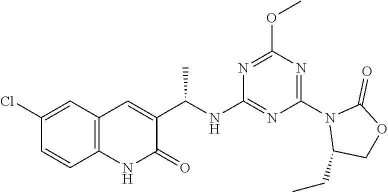 [SMILES: CC[C@H]1COC(=O)N1c1nc(N[C@@H](C)c2cc3cc(Cl)ccc3[nH]c2=O)nc(OC)n1] | 2.309 |
| 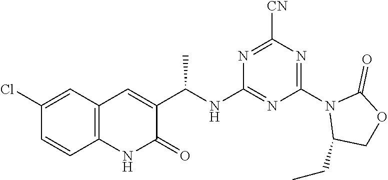 [SMILES: CC[C@H]1COC(=O)N1c1nc(C#N)nc(N[C@@H](C)c2cc3cc(Cl)ccc3[nH]c2=O)n1] | 2.310 |
| 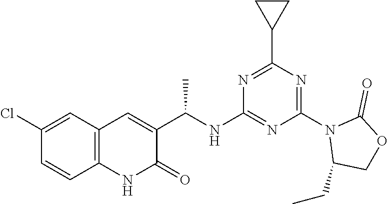 [SMILES: CC[C@H]1COC(=O)N1c1nc(N[C@@H](C)c2cc3cc(Cl)ccc3[nH]c2=O)nc(C2CC2)n1] | 2.311 |
| 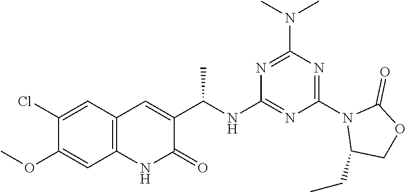 [SMILES: CC[C@H]1COC(=O)N1c1nc(N[C@@H](C)c2cc3cc(Cl)c(OC)cc3[nH]c2=O)nc(N(C)C)n1] | 2.312 |
| 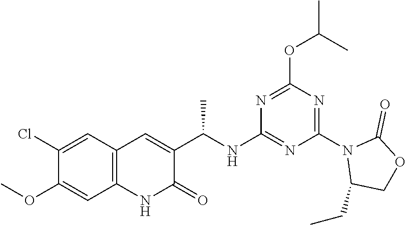 [SMILES: CC[C@H]1COC(=O)N1c1nc(N[C@@H](C)c2cc3cc(Cl)c(OC)cc3[nH]c2=O)nc(OC(C)C)n1] | 2.313 |
| 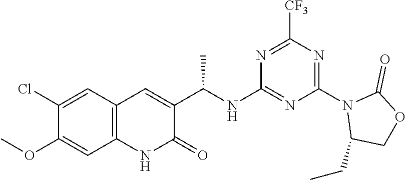 [SMILES: CC[C@H]1COC(=O)N1c1nc(N[C@@H](C)c2cc3cc(Cl)c(OC)cc3[nH]c2=O)nc(C(F)(F)F)n1] | 2.314 |
| 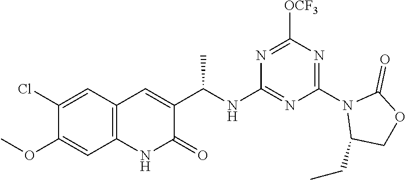 [SMILES: CC[C@H]1COC(=O)N1c1nc(N[C@@H](C)c2cc3cc(Cl)c(OC)cc3[nH]c2=O)nc(OC(F)(F)F)n1] | 2.315 |
| 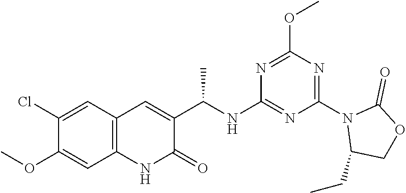 [SMILES: CC[C@H]1COC(=O)N1c1nc(N[C@@H](C)c2cc3cc(Cl)c(OC)cc3[nH]c2=O)nc(OC)n1] | 2.316 |
| 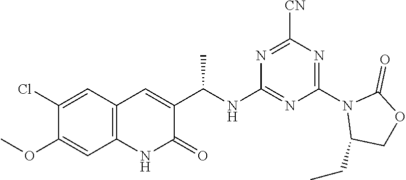 [SMILES: CC[C@H]1COC(=O)N1c1nc(C#N)nc(N[C@@H](C)c2cc3cc(Cl)c(OC)cc3[nH]c2=O)n1] | 2.317 |
| 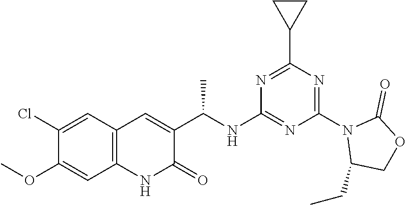 [SMILES: CC[C@H]1COC(=O)N1c1nc(N[C@@H](C)c2cc3cc(Cl)c(OC)cc3[nH]c2=O)nc(C2CC2)n1] | 2.318 |
| 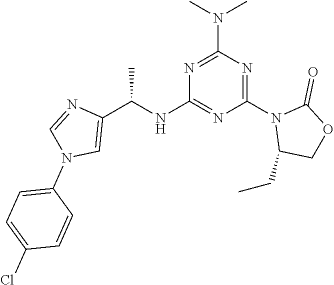 [SMILES: CC[C@H]1COC(=O)N1c1nc(N[C@@H](C)c2cn(-c3ccc(Cl)cc3)cn2)nc(N(C)C)n1] | 2.319 |
| 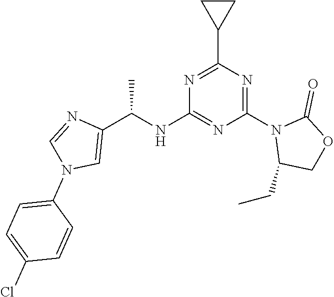 [SMILES: CC[C@H]1COC(=O)N1c1nc(N[C@@H](C)c2cn(-c3ccc(Cl)cc3)cn2)nc(C2CC2)n1] | 2.320 |
| 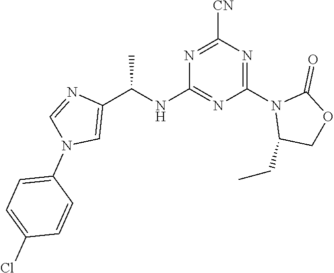 [SMILES: CC[C@H]1COC(=O)N1c1nc(C#N)nc(N[C@@H](C)c2cn(-c3ccc(Cl)cc3)cn2)n1] | 2.321 |
| 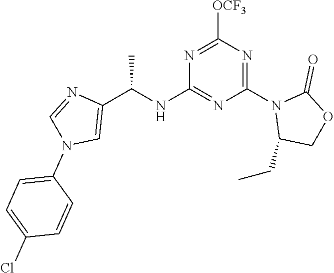 [SMILES: CC[C@H]1COC(=O)N1c1nc(N[C@@H](C)c2cn(-c3ccc(Cl)cc3)cn2)nc(OC(F)(F)F)n1] | 2.322 |
| 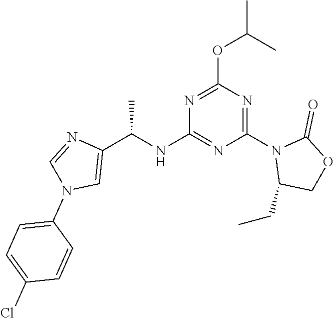 [SMILES: CC[C@H]1COC(=O)N1c1nc(N[C@@H](C)c2cn(-c3ccc(Cl)cc3)cn2)nc(OC(C)C)n1] | 2.323 |
| 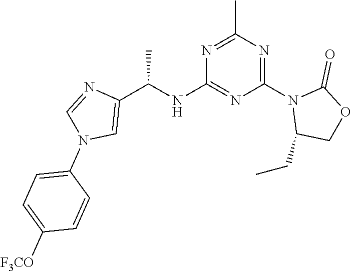 [SMILES: CC[C@H]1COC(=O)N1c1nc(C)nc(N[C@@H](C)c2cn(-c3ccc(OC(F)(F)F)cc3)cn2)n1] | 2.324 |
| 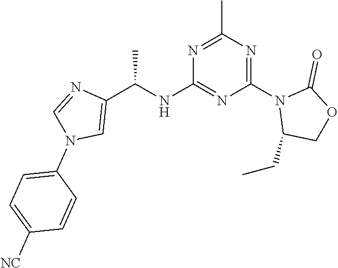 [SMILES: CC[C@H]1COC(=O)N1c1nc(C)nc(N[C@@H](C)c2cn(-c3ccc(C#N)cc3)cn2)n1] | 2.325 |
| 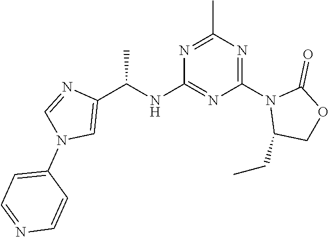 [SMILES: CC[C@H]1COC(=O)N1c1nc(C)nc(N[C@@H](C)c2cn(-c3ccncc3)cn2)n1] | 2.326 |
| 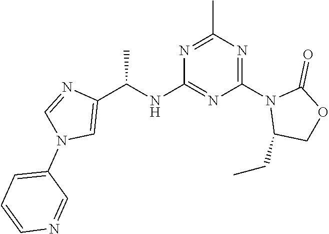 [SMILES: CC[C@H]1COC(=O)N1c1nc(C)nc(N[C@@H](C)c2cn(-c3cccnc3)cn2)n1] | 2.327 |
| 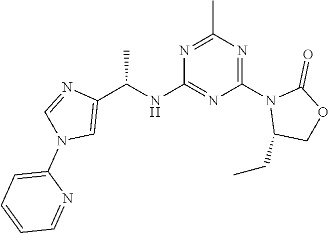 [SMILES: CC[C@H]1COC(=O)N1c1nc(C)nc(N[C@@H](C)c2cn(-c3ccccn3)cn2)n1] | 2.328 |
| 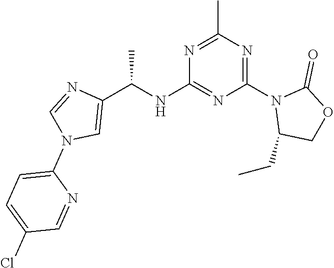 [SMILES: CC[C@H]1COC(=O)N1c1nc(C)nc(N[C@@H](C)c2cn(-c3ccc(Cl)cn3)cn2)n1] | 2.329 |
| 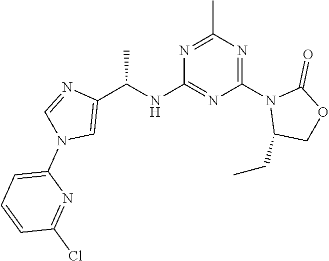 [SMILES: CC[C@H]1COC(=O)N1c1nc(C)nc(N[C@@H](C)c2cn(-c3cccc(Cl)n3)cn2)n1] | 2.330 |
| 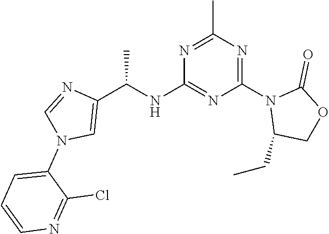 [SMILES: CC[C@H]1COC(=O)N1c1nc(C)nc(N[C@@H](C)c2cn(-c3cccnc3Cl)cn2)n1] | 2.331 |
| 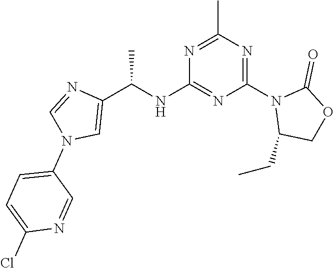 [SMILES: CC[C@H]1COC(=O)N1c1nc(C)nc(N[C@@H](C)c2cn(-c3ccc(Cl)nc3)cn2)n1] | 2.332 |
| 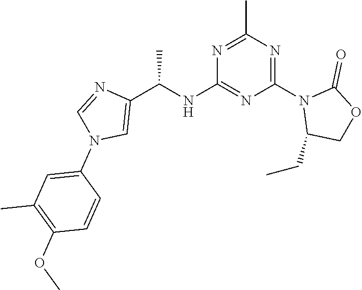 [SMILES: CC[C@H]1COC(=O)N1c1nc(C)nc(N[C@@H](C)c2cn(-c3ccc(OC)c(C)c3)cn2)n1] | 2.333 |
| 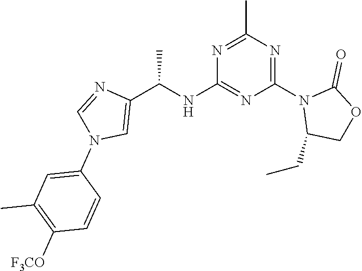 [SMILES: CC[C@H]1COC(=O)N1c1nc(C)nc(N[C@@H](C)c2cn(-c3ccc(OC(F)(F)F)c(C)c3)cn2)n1] | 2.334 |
| 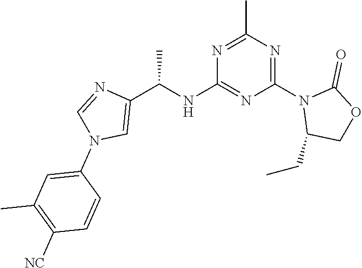 [SMILES: CC[C@H]1COC(=O)N1c1nc(C)nc(N[C@@H](C)c2cn(-c3ccc(C#N)c(C)c3)cn2)n1] | 2.335 |
| 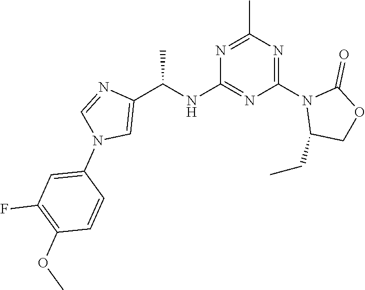 [SMILES: CC[C@H]1COC(=O)N1c1nc(C)nc(N[C@@H](C)c2cn(-c3ccc(OC)c(F)c3)cn2)n1] | 2.336 |
| 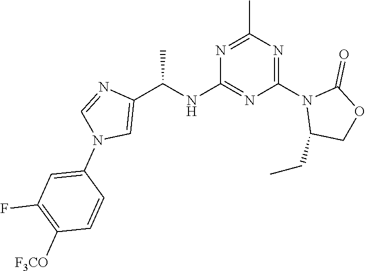 [SMILES: CC[C@H]1COC(=O)N1c1nc(C)nc(N[C@@H](C)c2cn(-c3ccc(OC(F)(F)F)c(F)c3)cn2)n1] | 2.337 |
| 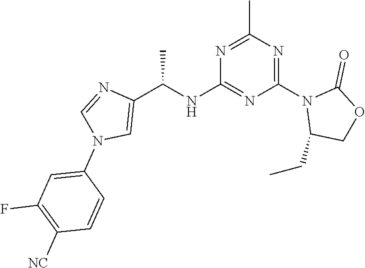 [SMILES: CC[C@H]1COC(=O)N1c1nc(C)nc(N[C@@H](C)c2cn(-c3ccc(C#N)c(F)c3)cn2)n1] | 2.338 |
| 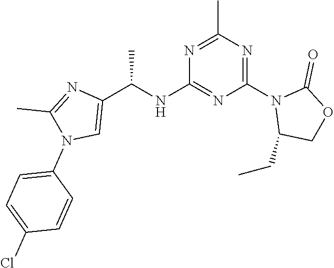 [SMILES: CC[C@H]1COC(=O)N1c1nc(C)nc(N[C@@H](C)c2cn(-c3ccc(Cl)cc3)c(C)n2)n1] | 2.339 |
| 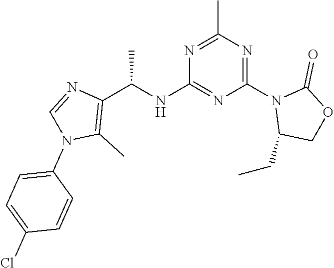 [SMILES: CC[C@H]1COC(=O)N1c1nc(C)nc(N[C@@H](C)c2ncn(-c3ccc(Cl)cc3)c2C)n1] | 2.340 |
| 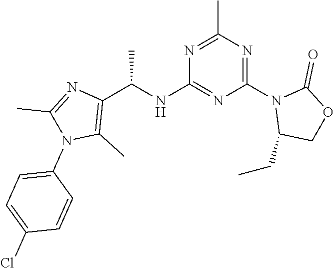 [SMILES: CC[C@H]1COC(=O)N1c1nc(C)nc(N[C@@H](C)c2nc(C)n(-c3ccc(Cl)cc3)c2C)n1] | 2.341 |
| 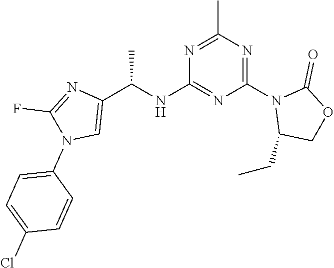 [SMILES: CC[C@H]1COC(=O)N1c1nc(C)nc(N[C@@H](C)c2cn(-c3ccc(Cl)cc3)c(F)n2)n1] | 2.342 |
| 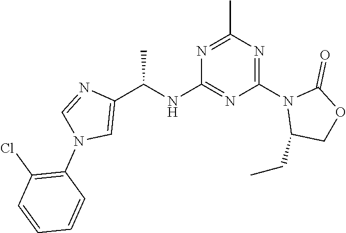 [SMILES: CC[C@H]1COC(=O)N1c1nc(C)nc(N[C@@H](C)c2cn(-c3ccccc3Cl)cn2)n1] | 2.343 |
| 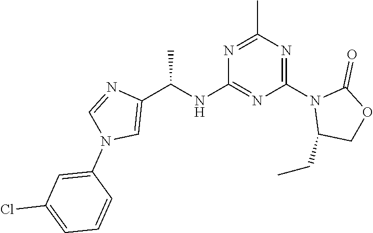 [SMILES: CC[C@H]1COC(=O)N1c1nc(C)nc(N[C@@H](C)c2cn(-c3cccc(Cl)c3)cn2)n1] | 2.344 |
| 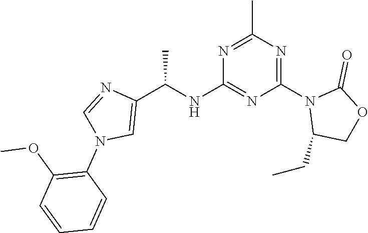 [SMILES: CC[C@H]1COC(=O)N1c1nc(C)nc(N[C@@H](C)c2cn(-c3ccccc3OC)cn2)n1] | 2.345 |
| 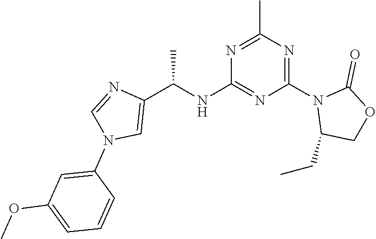 [SMILES: CC[C@H]1COC(=O)N1c1nc(C)nc(N[C@@H](C)c2cn(-c3cccc(OC)c3)cn2)n1] | 2.346 |
| 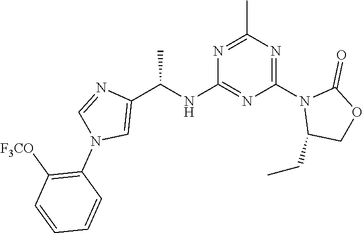 [SMILES: CC[C@H]1COC(=O)N1c1nc(C)nc(N[C@@H](C)c2cn(-c3ccccc3OC(F)(F)F)cn2)n1] | 2.347 |
| 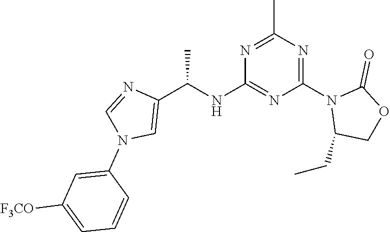 [SMILES: CC[C@H]1COC(=O)N1c1nc(C)nc(N[C@@H](C)c2cn(-c3cccc(OC(F)(F)F)c3)cn2)n1] | 2.348 |
| 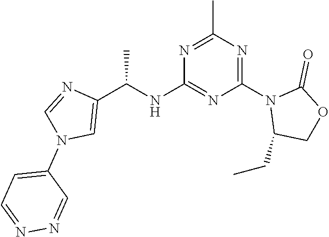 [SMILES: CC[C@H]1COC(=O)N1c1nc(C)nc(N[C@@H](C)c2cn(-c3ccnnc3)cn2)n1] | 2.349 |
| 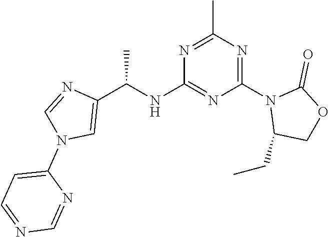 [SMILES: CC[C@H]1COC(=O)N1c1nc(C)nc(N[C@@H](C)c2cn(-c3ccncn3)cn2)n1] | 2.350 |
| 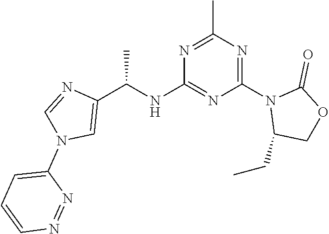 [SMILES: CC[C@H]1COC(=O)N1c1nc(C)nc(N[C@@H](C)c2cn(-c3cccnn3)cn2)n1] | 2.351 |
| 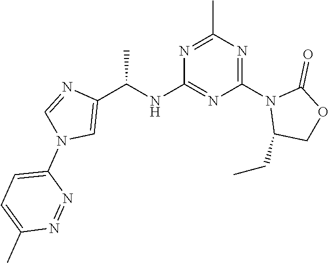 [SMILES: CC[C@H]1COC(=O)N1c1nc(C)nc(N[C@@H](C)c2cn(-c3ccc(C)nn3)cn2)n1] | 2.352 |
| 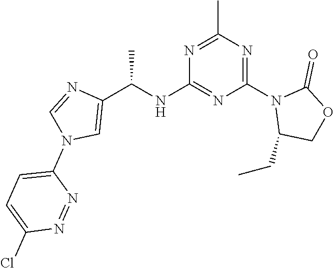 [SMILES: CC[C@H]1COC(=O)N1c1nc(C)nc(N[C@@H](C)c2cn(-c3ccc(Cl)nn3)cn2)n1] | 2.353 |
| 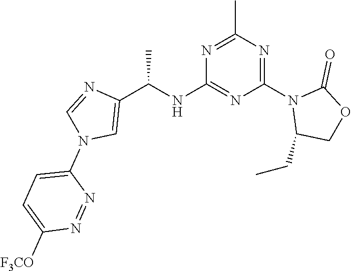 [SMILES: CC[C@H]1COC(=O)N1c1nc(C)nc(N[C@@H](C)c2cn(-c3ccc(OC(F)(F)F)nn3)cn2)n1] | 2.354 |
| 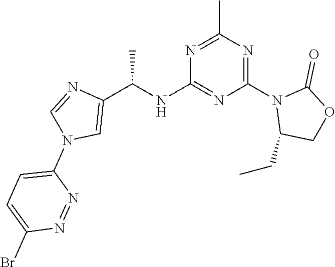 [SMILES: CC[C@H]1COC(=O)N1c1nc(C)nc(N[C@@H](C)c2cn(-c3ccc(Br)nn3)cn2)n1] | 2.355 |
| 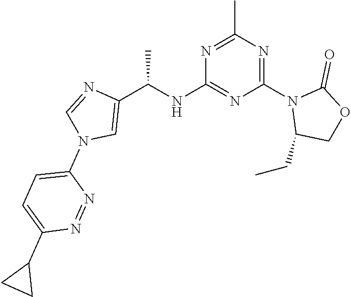 [SMILES: CC[C@H]1COC(=O)N1c1nc(C)nc(N[C@@H](C)c2cn(-c3ccc(C4CC4)nn3)cn2)n1] | 2.356 |
| 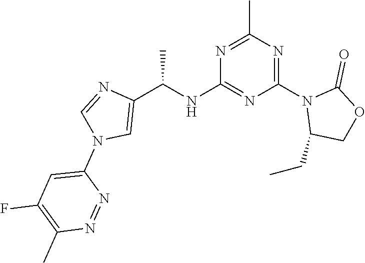 [SMILES: CC[C@H]1COC(=O)N1c1nc(C)nc(N[C@@H](C)c2cn(-c3cc(F)c(C)nn3)cn2)n1] | 2.357 |
| 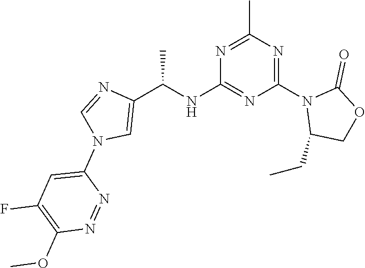 [SMILES: CC[C@H]1COC(=O)N1c1nc(C)nc(N[C@@H](C)c2cn(-c3cc(F)c(OC)nn3)cn2)n1] | 2.358 |
| 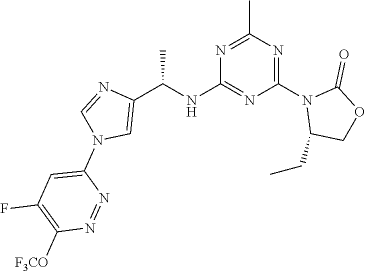 [SMILES: CC[C@H]1COC(=O)N1c1nc(C)nc(N[C@@H](C)c2cn(-c3cc(F)c(OC(F)(F)F)nn3)cn2)n1] | 2.359 |
| 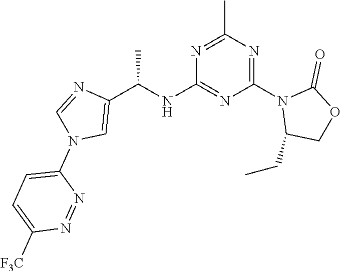 [SMILES: CC[C@H]1COC(=O)N1c1nc(C)nc(N[C@@H](C)c2cn(-c3ccc(C(F)(F)F)nn3)cn2)n1] | 2.360 |
| 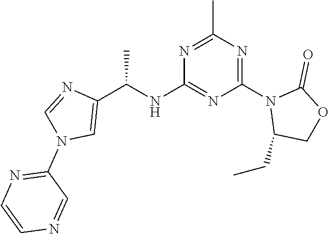 [SMILES: CC[C@H]1COC(=O)N1c1nc(C)nc(N[C@@H](C)c2cn(-c3cnccn3)cn2)n1] | 2.361 |
| 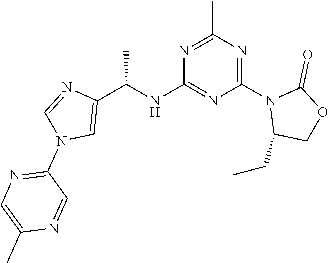 [SMILES: CC[C@H]1COC(=O)N1c1nc(C)nc(N[C@@H](C)c2cn(-c3cnc(C)cn3)cn2)n1] | 2.362 |
| 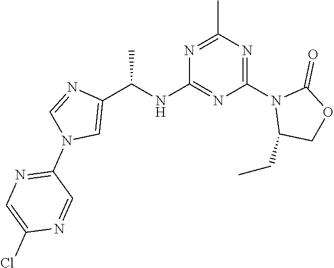 [SMILES: CC[C@H]1COC(=O)N1c1nc(C)nc(N[C@@H](C)c2cn(-c3cnc(Cl)cn3)cn2)n1] | 2.363 |
| 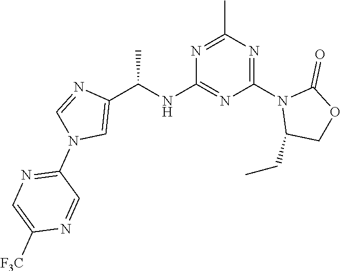 [SMILES: CC[C@H]1COC(=O)N1c1nc(C)nc(N[C@@H](C)c2cn(-c3cnc(C(F)(F)F)cn3)cn2)n1] | 2.364 |
| 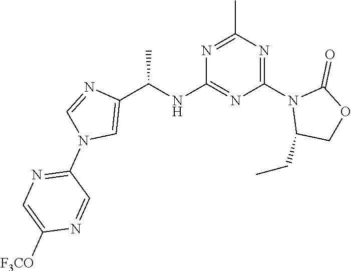 [SMILES: CC[C@H]1COC(=O)N1c1nc(C)nc(N[C@@H](C)c2cn(-c3cnc(OC(F)(F)F)cn3)cn2)n1] | 2.365 |
| 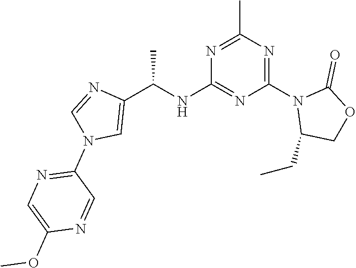 [SMILES: CC[C@H]1COC(=O)N1c1nc(C)nc(N[C@@H](C)c2cn(-c3cnc(OC)cn3)cn2)n1] | 2.366 |
| 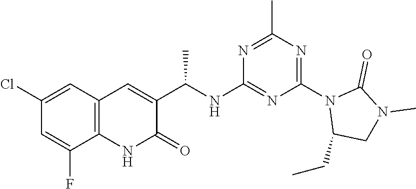 [SMILES: CC[C@H]1CN(C)C(=O)N1c1nc(C)nc(N[C@@H](C)c2cc3cc(Cl)cc(F)c3[nH]c2=O)n1] | 2.367 |
| 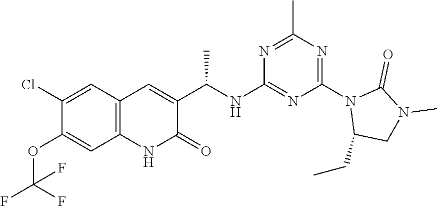 [SMILES: CC[C@H]1CN(C)C(=O)N1c1nc(C)nc(N[C@@H](C)c2cc3cc(Cl)c(OC(F)(F)F)cc3[nH]c2=O)n1] | 2.368 |
| 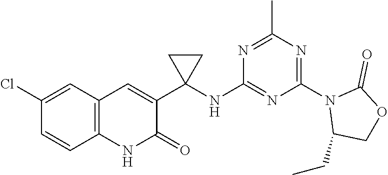 [SMILES: CC[C@H]1COC(=O)N1c1nc(C)nc(NC2(c3cc4cc(Cl)ccc4[nH]c3=O)CC2)n1] | 2.369 |
| 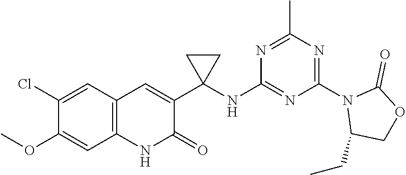 [SMILES: CC[C@H]1COC(=O)N1c1nc(C)nc(NC2(c3cc4cc(Cl)c(OC)cc4[nH]c3=O)CC2)n1] | 2.370 |
| 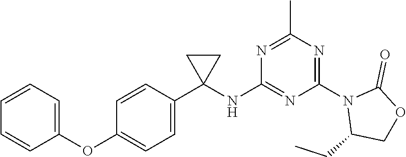 [SMILES: CC[C@H]1COC(=O)N1c1nc(C)nc(NC2(c3ccc(Oc4ccccc4)cc3)CC2)n1] | 2.371 |
| 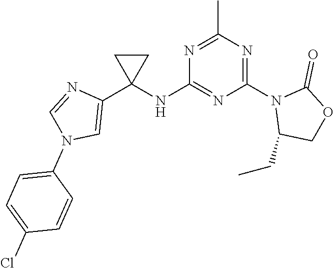 [SMILES: CC[C@H]1COC(=O)N1c1nc(C)nc(NC2(c3cn(-c4ccc(Cl)cc4)cn3)CC2)n1] | 2.372 |
| 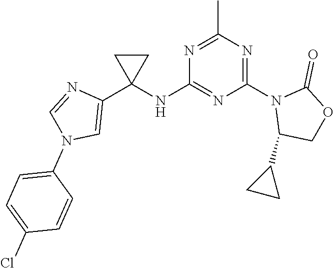 [SMILES: Cc1nc(NC2(c3cn(-c4ccc(Cl)cc4)cn3)CC2)nc(N2C(=O)OC[C@@H]2C2CC2)n1] | 2.373 |
| 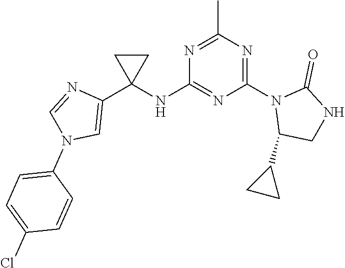 [SMILES: Cc1nc(NC2(c3cn(-c4ccc(Cl)cc4)cn3)CC2)nc(N2C(=O)NC[C@@H]2C2CC2)n1] | 2.374 |
| 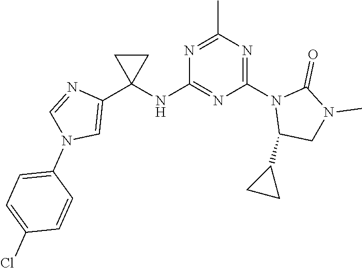 [SMILES: Cc1nc(NC2(c3cn(-c4ccc(Cl)cc4)cn3)CC2)nc(N2C(=O)N(C)C[C@@H]2C2CC2)n1] | 2.375 |
| 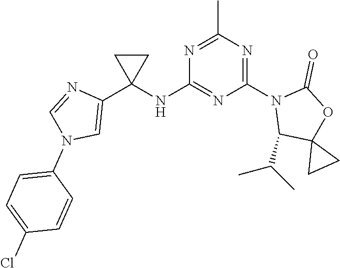 [SMILES: Cc1nc(NC2(c3cn(-c4ccc(Cl)cc4)cn3)CC2)nc(N2C(=O)OC3(CC3)[C@@H]2C(C)C)n1] | 2.376 |
| 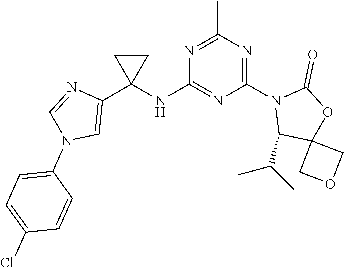 [SMILES: Cc1nc(NC2(c3cn(-c4ccc(Cl)cc4)cn3)CC2)nc(N2C(=O)OC3(COC3)[C@@H]2C(C)C)n1] | 2.377 |
| 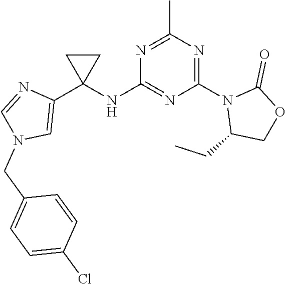 [SMILES: CC[C@H]1COC(=O)N1c1nc(C)nc(NC2(c3cn(Cc4ccc(Cl)cc4)cn3)CC2)n1] | 2.378 |
| 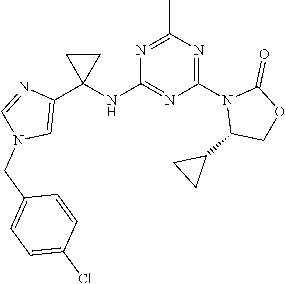 [SMILES: Cc1nc(NC2(c3cn(Cc4ccc(Cl)cc4)cn3)CC2)nc(N2C(=O)OC[C@@H]2C2CC2)n1] | 2.379 |
| 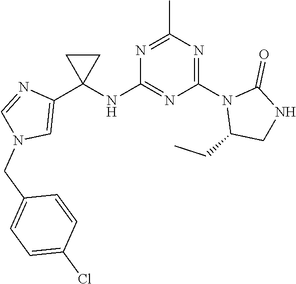 [SMILES: CC[C@H]1CNC(=O)N1c1nc(C)nc(NC2(c3cn(Cc4ccc(Cl)cc4)cn3)CC2)n1] | 2.380 |
| 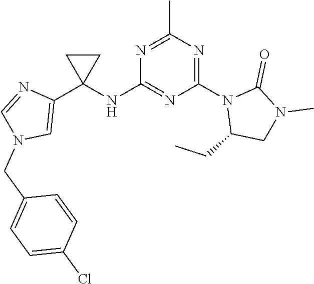 [SMILES: CC[C@H]1CN(C)C(=O)N1c1nc(C)nc(NC2(c3cn(Cc4ccc(Cl)cc4)cn3)CC2)n1] | 2.381 |
| 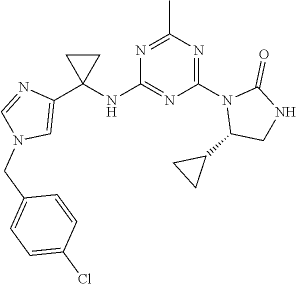 [SMILES: Cc1nc(NC2(c3cn(Cc4ccc(Cl)cc4)cn3)CC2)nc(N2C(=O)NC[C@@H]2C2CC2)n1] | 2.382 |
| 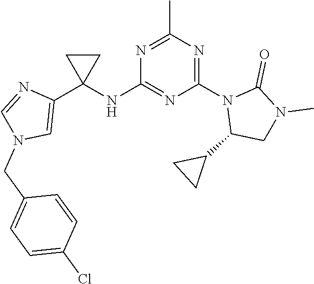 [SMILES: Cc1nc(NC2(c3cn(Cc4ccc(Cl)cc4)cn3)CC2)nc(N2C(=O)N(C)C[C@@H]2C2CC2)n1] | 2.383 |
| 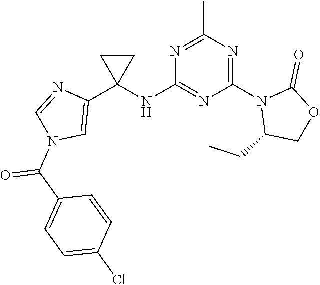 [SMILES: CC[C@H]1COC(=O)N1c1nc(C)nc(NC2(c3cn(C(=O)c4ccc(Cl)cc4)cn3)CC2)n1] | 2.384 |
| 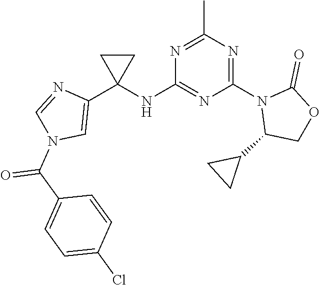 [SMILES: Cc1nc(NC2(c3cn(C(=O)c4ccc(Cl)cc4)cn3)CC2)nc(N2C(=O)OC[C@@H]2C2CC2)n1] | 2.385 |
| 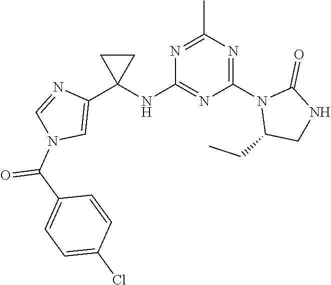 [SMILES: CC[C@H]1CNC(=O)N1c1nc(C)nc(NC2(c3cn(C(=O)c4ccc(Cl)cc4)cn3)CC2)n1] | 2.386 |
| 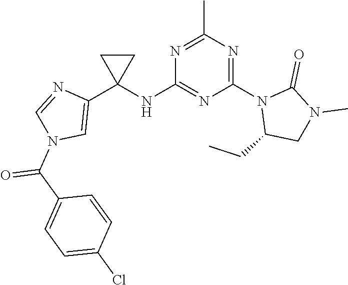 [SMILES: CC[C@H]1CN(C)C(=O)N1c1nc(C)nc(NC2(c3cn(C(=O)c4ccc(Cl)cc4)cn3)CC2)n1] | 2.387 |
| 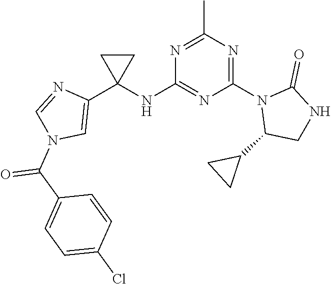 [SMILES: Cc1nc(NC2(c3cn(C(=O)c4ccc(Cl)cc4)cn3)CC2)nc(N2C(=O)NC[C@@H]2C2CC2)n1] | 2.388 |
| 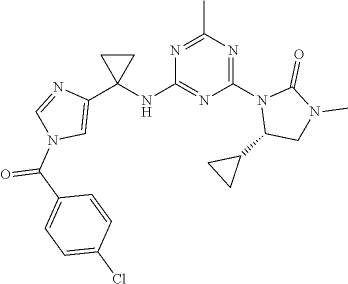 [SMILES: Cc1nc(NC2(c3cn(C(=O)c4ccc(Cl)cc4)cn3)CC2)nc(N2C(=O)N(C)C[C@@H]2C2CC2)n1] | 2.389 |
| 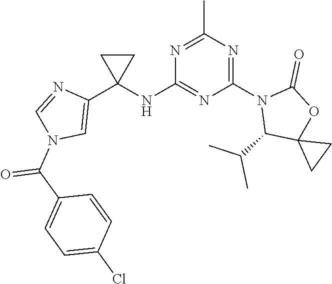 [SMILES: Cc1nc(NC2(c3cn(C(=O)c4ccc(Cl)cc4)cn3)CC2)nc(N2C(=O)OC3(CC3)[C@@H]2C(C)C)n1] | 2.390 |
| 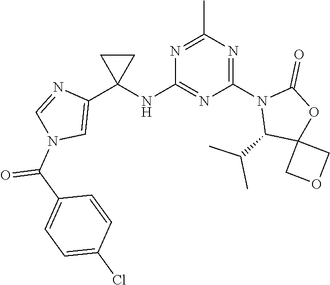 [SMILES: Cc1nc(NC2(c3cn(C(=O)c4ccc(Cl)cc4)cn3)CC2)nc(N2C(=O)OC3(COC3)[C@@H]2C(C)C)n1] | 2.391 |
| 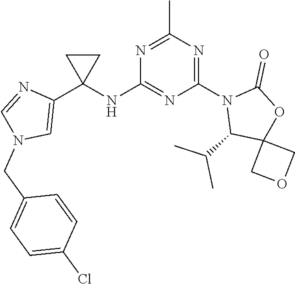 [SMILES: Cc1nc(NC2(c3cn(Cc4ccc(Cl)cc4)cn3)CC2)nc(N2C(=O)OC3(COC3)[C@@H]2C(C)C)n1] | 2.392 |
| 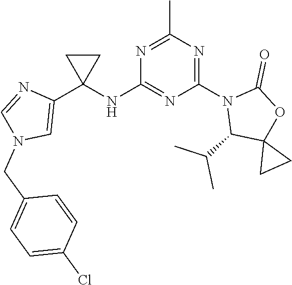 [SMILES: Cc1nc(NC2(c3cn(Cc4ccc(Cl)cc4)cn3)CC2)nc(N2C(=O)OC3(CC3)[C@@H]2C(C)C)n1] | 2.393 |
| 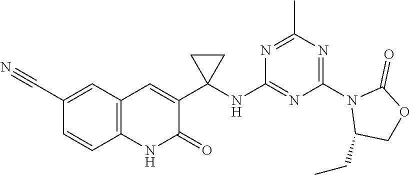 [SMILES: CC[C@H]1COC(=O)N1c1nc(C)nc(NC2(c3cc4cc(C#N)ccc4[nH]c3=O)CC2)n1] | 2.394 |
| 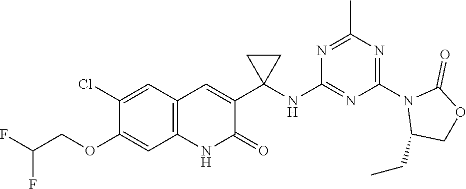 [SMILES: CC[C@H]1COC(=O)N1c1nc(C)nc(NC2(c3cc4cc(Cl)c(OCC(F)F)cc4[nH]c3=O)CC2)n1] | 2.395 |
| 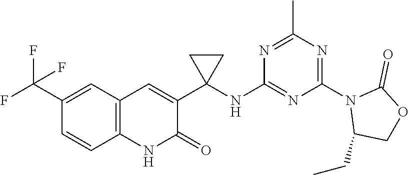 [SMILES: CC[C@H]1COC(=O)N1c1nc(C)nc(NC2(c3cc4cc(C(F)(F)F)ccc4[nH]c3=O)CC2)n1] | 2.396 |
| 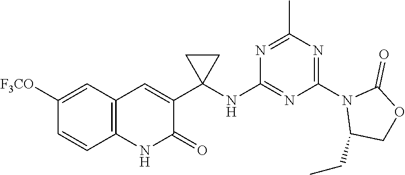 [SMILES: CC[C@H]1COC(=O)N1c1nc(C)nc(NC2(c3cc4cc(OC(F)(F)F)ccc4[nH]c3=O)CC2)n1] | 2.397 |
| 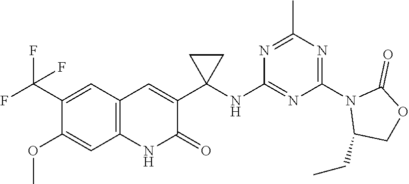 [SMILES: CC[C@H]1COC(=O)N1c1nc(C)nc(NC2(c3cc4cc(C(F)(F)F)c(OC)cc4[nH]c3=O)CC2)n1] | 2.398 |
| 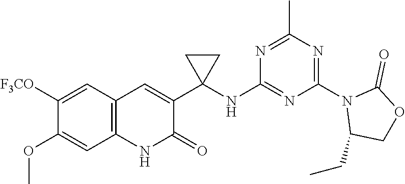 [SMILES: CC[C@H]1COC(=O)N1c1nc(C)nc(NC2(c3cc4cc(OC(F)(F)F)c(OC)cc4[nH]c3=O)CC2)n1] | 2.399 |
| 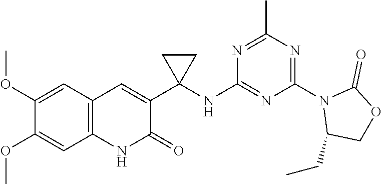 [SMILES: CC[C@H]1COC(=O)N1c1nc(C)nc(NC2(c3cc4cc(OC)c(OC)cc4[nH]c3=O)CC2)n1] | 2.400 |
| 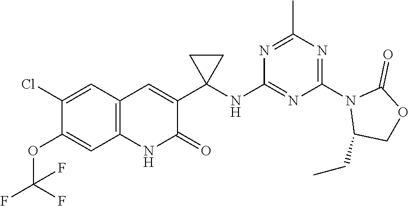 [SMILES: CC[C@H]1COC(=O)N1c1nc(C)nc(NC2(c3cc4cc(Cl)c(OC(F)(F)F)cc4[nH]c3=O)CC2)n1] | 2.401 |
| 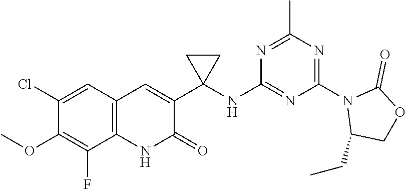 [SMILES: CC[C@H]1COC(=O)N1c1nc(C)nc(NC2(c3cc4cc(Cl)c(OC)c(F)c4[nH]c3=O)CC2)n1] | 2.402 |
| 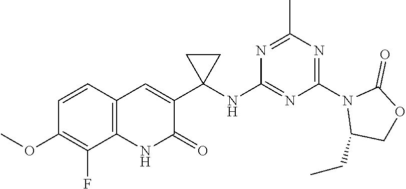 [SMILES: CC[C@H]1COC(=O)N1c1nc(C)nc(NC2(c3cc4ccc(OC)c(F)c4[nH]c3=O)CC2)n1] | 2.403 |
| 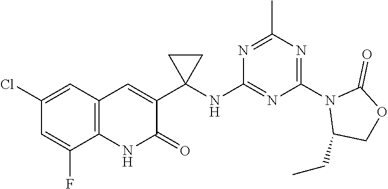 [SMILES: CC[C@H]1COC(=O)N1c1nc(C)nc(NC2(c3cc4cc(Cl)cc(F)c4[nH]c3=O)CC2)n1] | 2.404 |
| 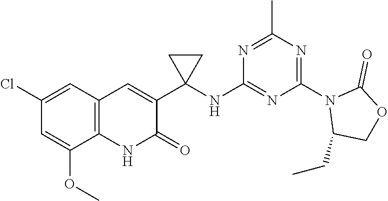 [SMILES: CC[C@H]1COC(=O)N1c1nc(C)nc(NC2(c3cc4cc(Cl)cc(OC)c4[nH]c3=O)CC2)n1] | 2.405 |
| 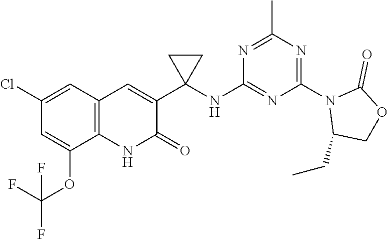 [SMILES: CC[C@H]1COC(=O)N1c1nc(C)nc(NC2(c3cc4cc(Cl)cc(OC(F)(F)F)c4[nH]c3=O)CC2)n1] | 2.406 |
| 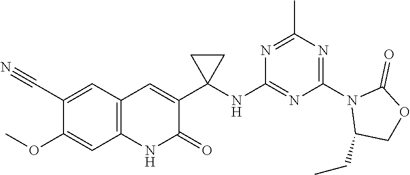 [SMILES: CC[C@H]1COC(=O)N1c1nc(C)nc(NC2(c3cc4cc(C#N)c(OC)cc4[nH]c3=O)CC2)n1] | 2.407 |
| 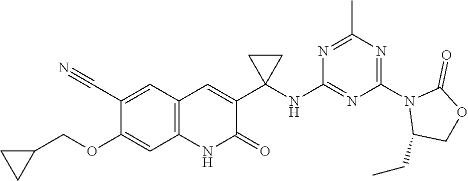 [SMILES: CC[C@H]1COC(=O)N1c1nc(C)nc(NC2(c3cc4cc(C#N)c(OCC5CC5)cc4[nH]c3=O)CC2)n1] | 2.408 |
| 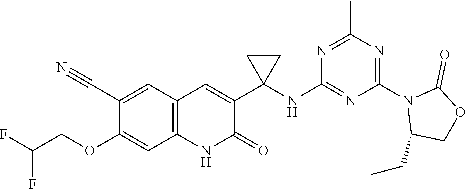 [SMILES: CC[C@H]1COC(=O)N1c1nc(C)nc(NC2(c3cc4cc(C#N)c(OCC(F)F)cc4[nH]c3=O)CC2)n1] | 2.409 |
| 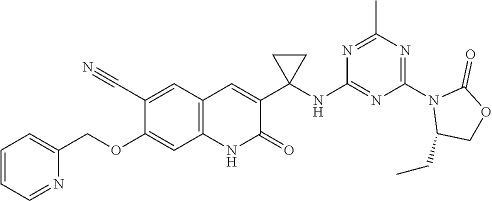 [SMILES: CC[C@H]1COC(=O)N1c1nc(C)nc(NC2(c3cc4cc(C#N)c(OCc5ccccn5)cc4[nH]c3=O)CC2)n1] | 2.410 |
| 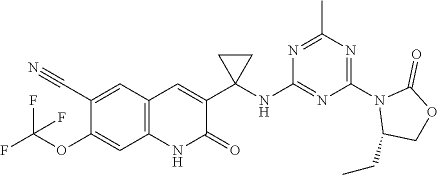 [SMILES: CC[C@H]1COC(=O)N1c1nc(C)nc(NC2(c3cc4cc(C#N)c(OC(F)(F)F)cc4[nH]c3=O)CC2)n1] | 2.411 |
| 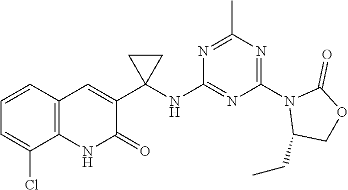 [SMILES: CC[C@H]1COC(=O)N1c1nc(C)nc(NC2(c3cc4cccc(Cl)c4[nH]c3=O)CC2)n1] | 2.412 |
| 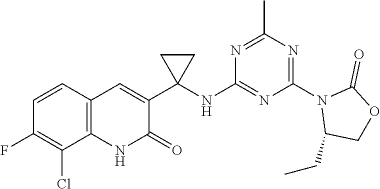 [SMILES: CC[C@H]1COC(=O)N1c1nc(C)nc(NC2(c3cc4ccc(F)c(Cl)c4[nH]c3=O)CC2)n1] | 2.413 |
| 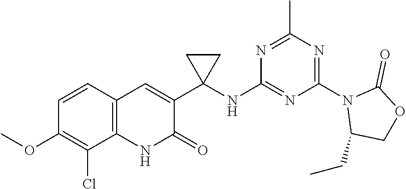 [SMILES: CC[C@H]1COC(=O)N1c1nc(C)nc(NC2(c3cc4ccc(OC)c(Cl)c4[nH]c3=O)CC2)n1] | 2.414 |
| 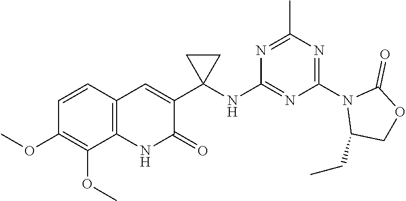 [SMILES: CC[C@H]1COC(=O)N1c1nc(C)nc(NC2(c3cc4ccc(OC)c(OC)c4[nH]c3=O)CC2)n1] | 2.415 |
| 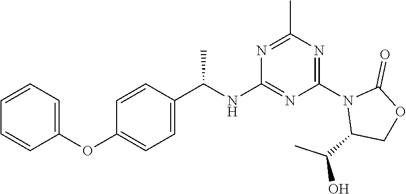 [SMILES: Cc1nc(N[C@@H](C)c2ccc(Oc3ccccc3)cc2)nc(N2C(=O)OC[C@@H]2[C@H](C)O)n1] | 2.416 |
| 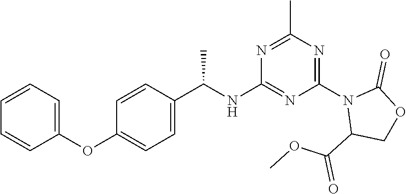 [SMILES: COC(=O)C1COC(=O)N1c1nc(C)nc(N[C@@H](C)c2ccc(Oc3ccccc3)cc2)n1] | 2.417 |
| 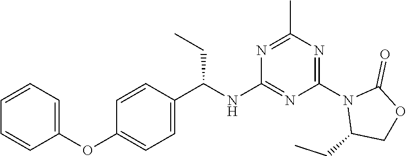 [SMILES: CC[C@H]1COC(=O)N1c1nc(C)nc(N[C@@H](CC)c2ccc(Oc3ccccc3)cc2)n1] | 2.418 |
| 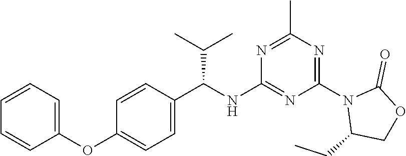 [SMILES: CC[C@H]1COC(=O)N1c1nc(C)nc(N[C@H](c2ccc(Oc3ccccc3)cc2)C(C)C)n1] | 2.419 |
| 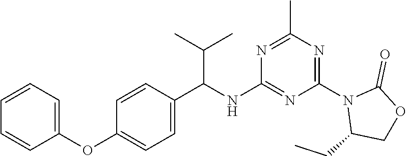 [SMILES: CC[C@H]1COC(=O)N1c1nc(C)nc(NC(c2ccc(Oc3ccccc3)cc2)C(C)C)n1] | 2.420 |
| 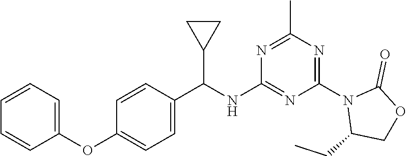 [SMILES: CC[C@H]1COC(=O)N1c1nc(C)nc(NC(c2ccc(Oc3ccccc3)cc2)C2CC2)n1] | 2.421 |
| 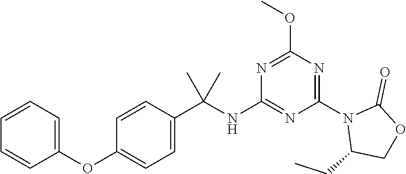 [SMILES: CC[C@H]1COC(=O)N1c1nc(NC(C)(C)c2ccc(Oc3ccccc3)cc2)nc(OC)n1] | 2.422 |
| 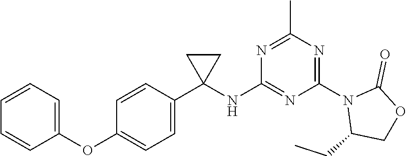 [SMILES: CC[C@H]1COC(=O)N1c1nc(C)nc(NC2(c3ccc(Oc4ccccc4)cc3)CC2)n1] | 2.423 |
| 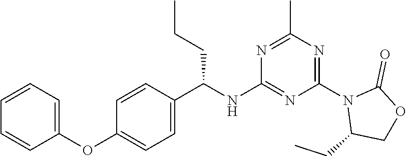 [SMILES: CCC[C@H](Nc1nc(C)nc(N2C(=O)OC[C@@H]2CC)n1)c1ccc(Oc2ccccc2)cc1] | 2.424 |
| 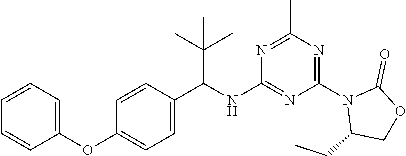 [SMILES: CC[C@H]1COC(=O)N1c1nc(C)nc(NC(c2ccc(Oc3ccccc3)cc2)C(C)(C)C)n1] | 2.425 |
| 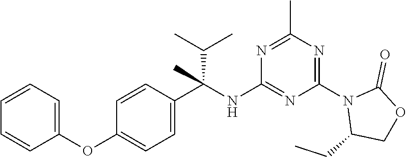 [SMILES: CC[C@H]1COC(=O)N1c1nc(C)nc(N[C@](C)(c2ccc(Oc3ccccc3)cc2)C(C)C)n1] | 2.426 |
| 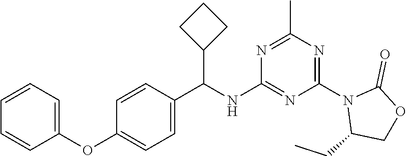 [SMILES: CC[C@H]1COC(=O)N1c1nc(C)nc(NC(c2ccc(Oc3ccccc3)cc2)C2CCC2)n1] | 2.427 |
| 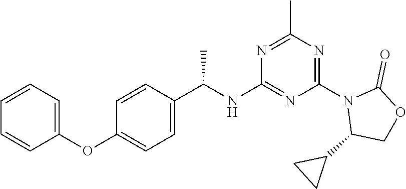 [SMILES: Cc1nc(N[C@@H](C)c2ccc(Oc3ccccc3)cc2)nc(N2C(=O)OC[C@@H]2C2CC2)n1] | 2.428 |
| 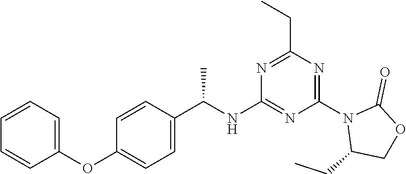 [SMILES: CCc1nc(N[C@@H](C)c2ccc(Oc3ccccc3)cc2)nc(N2C(=O)OC[C@@H]2CC)n1] | 2.429 |
| 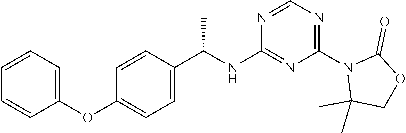 [SMILES: C[C@H](Nc1ncnc(N2C(=O)OCC2(C)C)n1)c1ccc(Oc2ccccc2)cc1] | 2.430 |
| 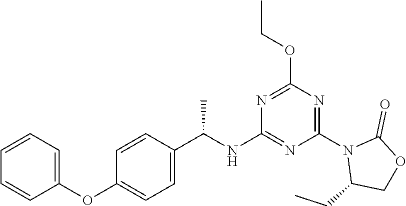 [SMILES: CCOc1nc(N[C@@H](C)c2ccc(Oc3ccccc3)cc2)nc(N2C(=O)OC[C@@H]2CC)n1] | 2.431 |
| 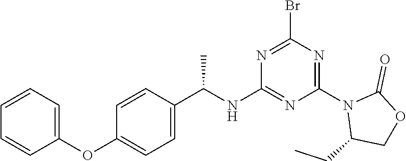 [SMILES: CC[C@H]1COC(=O)N1c1nc(Br)nc(N[C@@H](C)c2ccc(Oc3ccccc3)cc2)n1] | 2.432 |
| 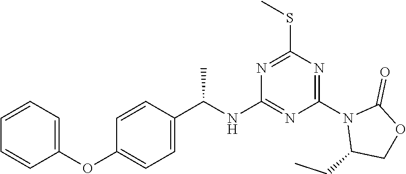 [SMILES: CC[C@H]1COC(=O)N1c1nc(N[C@@H](C)c2ccc(Oc3ccccc3)cc2)nc(SC)n1] | 2.433 |
| 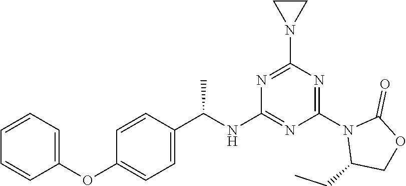 [SMILES: CC[C@H]1COC(=O)N1c1nc(N[C@@H](C)c2ccc(Oc3ccccc3)cc2)nc(N2CC2)n1] | 2.434 |
| 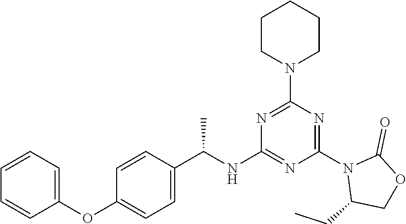 [SMILES: CC[C@H]1COC(=O)N1c1nc(N[C@@H](C)c2ccc(Oc3ccccc3)cc2)nc(N2CCCCC2)n1] | 2.435 |
| 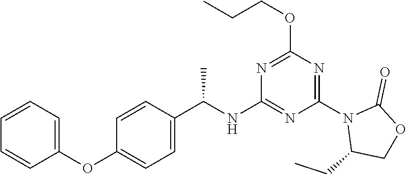 [SMILES: CCCOc1nc(N[C@@H](C)c2ccc(Oc3ccccc3)cc2)nc(N2C(=O)OC[C@@H]2CC)n1] | 2.436 |
| 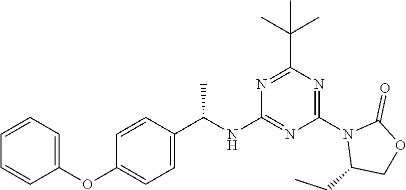 [SMILES: CC[C@H]1COC(=O)N1c1nc(N[C@@H](C)c2ccc(Oc3ccccc3)cc2)nc(C(C)(C)C)n1] | 2.437 |
| 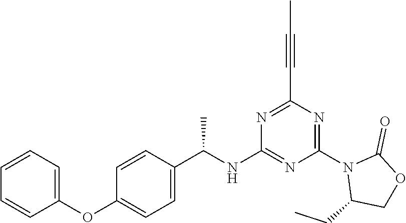 [SMILES: CC#Cc1nc(N[C@@H](C)c2ccc(Oc3ccccc3)cc2)nc(N2C(=O)OC[C@@H]2CC)n1] | 2.438 |
| 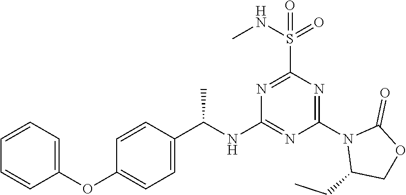 [SMILES: CC[C@H]1COC(=O)N1c1nc(N[C@@H](C)c2ccc(Oc3ccccc3)cc2)nc(S(=O)(=O)NC)n1] | 2.439 |
| 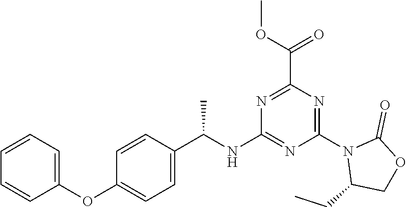 [SMILES: CC[C@H]1COC(=O)N1c1nc(N[C@@H](C)c2ccc(Oc3ccccc3)cc2)nc(C(=O)OC)n1] | 2.440 |
| 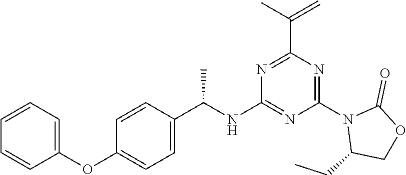 [SMILES: C=C(C)c1nc(N[C@@H](C)c2ccc(Oc3ccccc3)cc2)nc(N2C(=O)OC[C@@H]2CC)n1] | 2.441 |
| 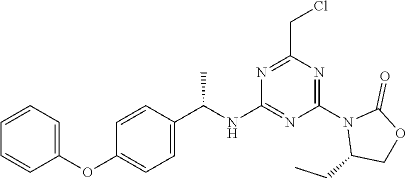 [SMILES: CC[C@H]1COC(=O)N1c1nc(CCl)nc(N[C@@H](C)c2ccc(Oc3ccccc3)cc2)n1] | 2.442 |
| 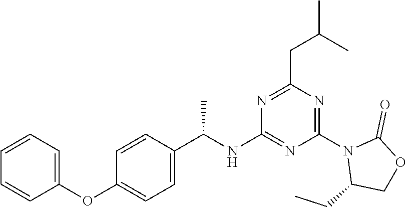 [SMILES: CC[C@H]1COC(=O)N1c1nc(CC(C)C)nc(N[C@@H](C)c2ccc(Oc3ccccc3)cc2)n1] | 2.443 |
| 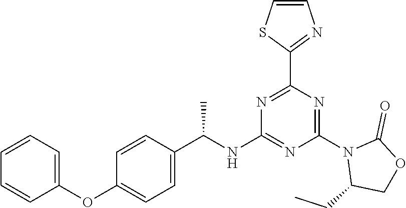 [SMILES: CC[C@H]1COC(=O)N1c1nc(N[C@@H](C)c2ccc(Oc3ccccc3)cc2)nc(-c2nccs2)n1] | 2.444 |
| 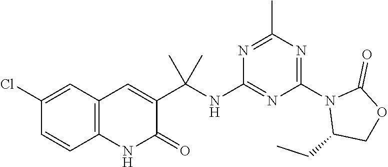 [SMILES: CC[C@H]1COC(=O)N1c1nc(C)nc(NC(C)(C)c2cc3cc(Cl)ccc3[nH]c2=O)n1] | 2.445 |
| 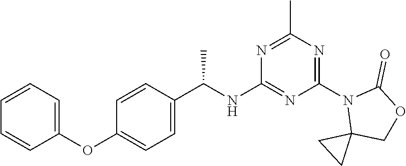 [SMILES: Cc1nc(N[C@@H](C)c2ccc(Oc3ccccc3)cc2)nc(N2C(=O)OCC23CC3)n1] | 2.446 |
| 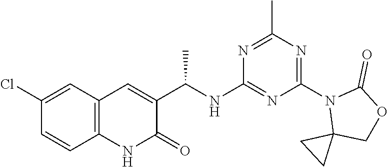 [SMILES: Cc1nc(N[C@@H](C)c2cc3cc(Cl)ccc3[nH]c2=O)nc(N2C(=O)OCC23CC3)n1] | 2.447 |
| 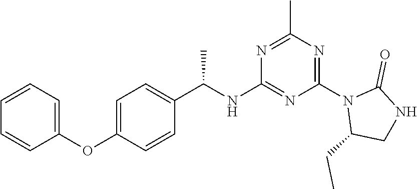 [SMILES: CC[C@H]1CNC(=O)N1c1nc(C)nc(N[C@@H](C)c2ccc(Oc3ccccc3)cc2)n1] | 2.448 |
| 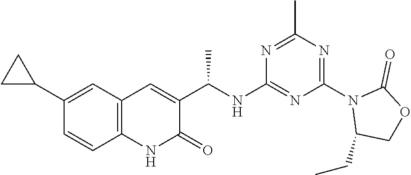 [SMILES: CC[C@H]1COC(=O)N1c1nc(C)nc(N[C@@H](C)c2cc3cc(C4CC4)ccc3[nH]c2=O)n1] | 2.449 |
| 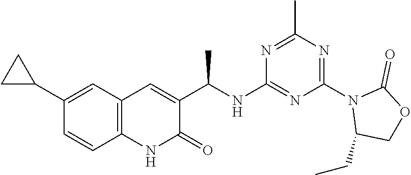 [SMILES: CC[C@H]1COC(=O)N1c1nc(C)nc(N[C@H](C)c2cc3cc(C4CC4)ccc3[nH]c2=O)n1] | 2.450 |
| 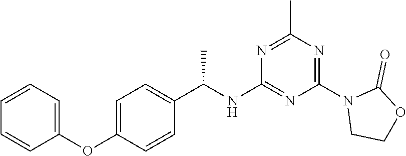 [SMILES: Cc1nc(N[C@@H](C)c2ccc(Oc3ccccc3)cc2)nc(N2CCOC2=O)n1] | 2.451 |
| 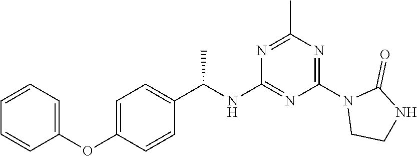 [SMILES: Cc1nc(N[C@@H](C)c2ccc(Oc3ccccc3)cc2)nc(N2CCNC2=O)n1] | 2.452 |
| 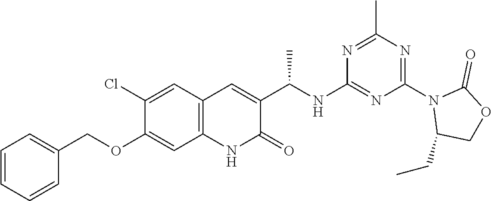 [SMILES: CC[C@H]1COC(=O)N1c1nc(C)nc(N[C@@H](C)c2cc3cc(Cl)c(OCc4ccccc4)cc3[nH]c2=O)n1] | 2.453 |
| 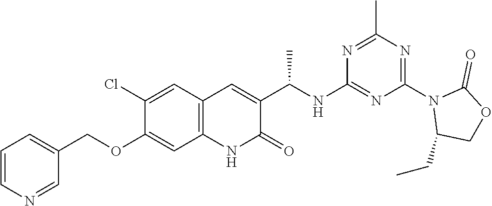 [SMILES: CC[C@H]1COC(=O)N1c1nc(C)nc(N[C@@H](C)c2cc3cc(Cl)c(OCc4cccnc4)cc3[nH]c2=O)n1] | 2.454 |
| 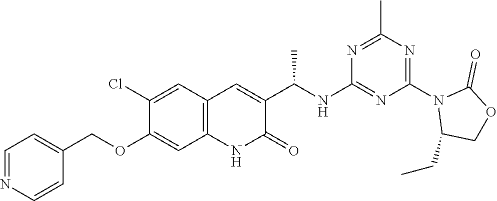 [SMILES: CC[C@H]1COC(=O)N1c1nc(C)nc(N[C@@H](C)c2cc3cc(Cl)c(OCc4ccncc4)cc3[nH]c2=O)n1] | 2.455 |
| 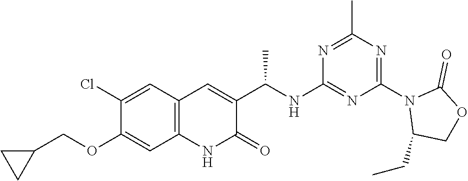 [SMILES: CC[C@H]1COC(=O)N1c1nc(C)nc(N[C@@H](C)c2cc3cc(Cl)c(OCC4CC4)cc3[nH]c2=O)n1] | 2.456 |
| 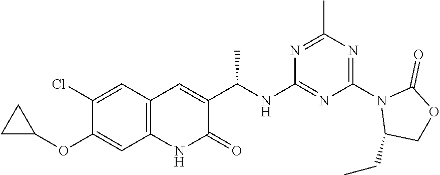 [SMILES: CC[C@H]1COC(=O)N1c1nc(C)nc(N[C@@H](C)c2cc3cc(Cl)c(OC4CC4)cc3[nH]c2=O)n1] | 2.457 |
| 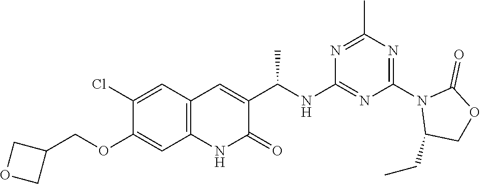 [SMILES: CC[C@H]1COC(=O)N1c1nc(C)nc(N[C@@H](C)c2cc3cc(Cl)c(OCC4COC4)cc3[nH]c2=O)n1] | 2.458 |
| 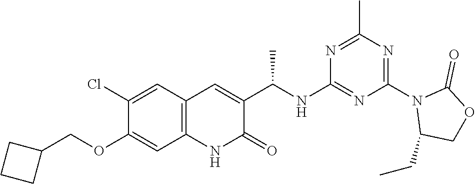 [SMILES: CC[C@H]1COC(=O)N1c1nc(C)nc(N[C@@H](C)c2cc3cc(Cl)c(OCC4CCC4)cc3[nH]c2=O)n1] | 2.459 |
| 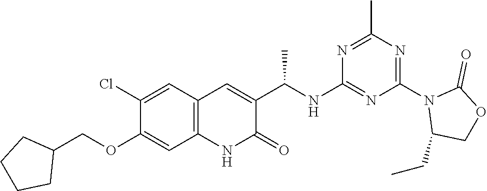 [SMILES: CC[C@H]1COC(=O)N1c1nc(C)nc(N[C@@H](C)c2cc3cc(Cl)c(OCC4CCCC4)cc3[nH]c2=O)n1] | 2.460 |
| 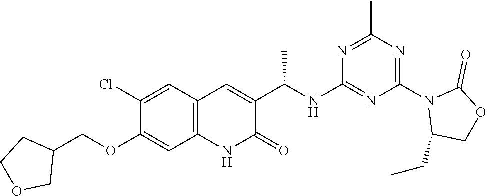 [SMILES: CC[C@H]1COC(=O)N1c1nc(C)nc(N[C@@H](C)c2cc3cc(Cl)c(OCC4CCOC4)cc3[nH]c2=O)n1] | 2.461 |
| 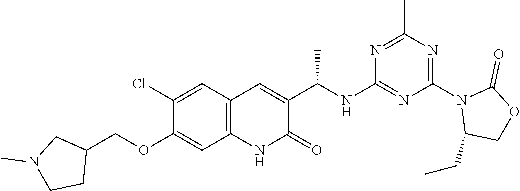 [SMILES: CC[C@H]1COC(=O)N1c1nc(C)nc(N[C@@H](C)c2cc3cc(Cl)c(OCC4CCN(C)C4)cc3[nH]c2=O)n1] | 2.462 |

In some embodiments, provided herein are compounds described in table-1 and table-2, or a salt, polymorph, solvate, enantiomer, stereoisomer or tautomer thereof, and uses thereof.

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds described herein may be used in the methods detailed herein, including, where applicable, intermediate compounds that may be isolated and administered to an individual.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. The structure or name is intended to embrace all possible stereoisomers of a compound depicted, and each unique stereoisomer has a compound number bearing a suffix “a”, “b”, etc. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (IA) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Methods

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a pharmaceutically acceptable salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate

In some embodiments, compounds of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)) may be synthesized according to general Scheme 1.

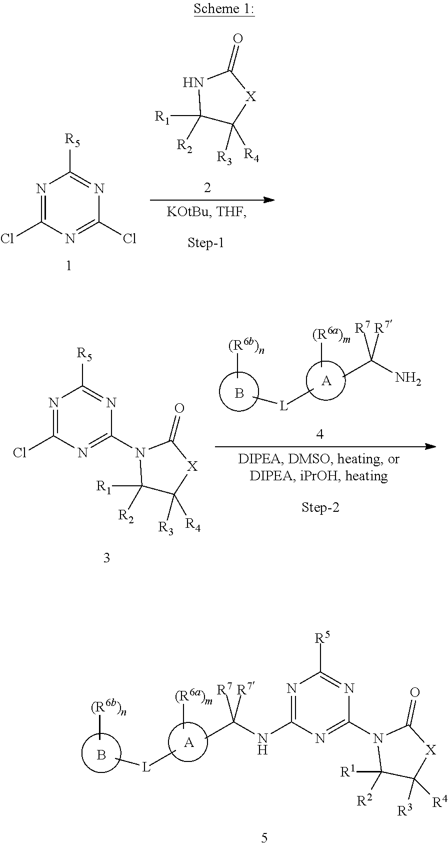

wherein X, A, B, L, R¹, R², R³, R⁴, R⁵, R^{6a}, R^{6b}, R⁷, R^{7′}, m and n are as defined for formula (IA), or any variation thereof detailed herein;

In some embodiments, compounds of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)) may be synthesized according to general Scheme 2.

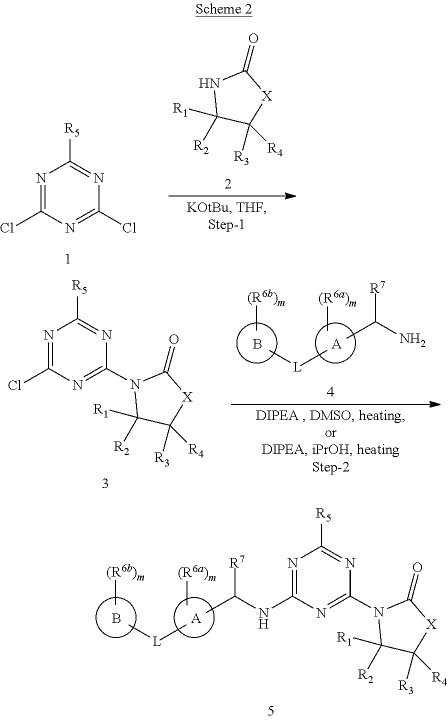

wherein A, B, L, R¹, R², R³, R⁴, R⁵, R^{6a}, R^{6b}, R⁷, m and n are as defined for formula (IA), or any variation thereof detailed herein; X is a heteroatom selected from O, S, NR^{a} or NH.

In some cases, stereoisomers are separated to give single enantiomers or diastereomers as single, unknown stereoisomers, and are arbitrarily drawn as single isomers. Where appropriate, information is given on separation method and elution time and order.

### UPLC-MS Standard Procedures

Analytical UPLC-MS was performed as described below. The masses (m/z) are reported from the positive mode electrospray ionization unless the negative mode is indicated.

### UPLC-MS Method-1

Instrument: Waters Acquity UPLC-MS SQD 3100; Column: Acquity UPLC BEH Shield RP 18, 1.7 μm, 2.1×50 mm; Eluent A: 0.05% TFA in Water, Eluent B: Acetonitrile; Gradient: 10% B to 50% B in 7 min, hold for 5 min, 50% B to 10% B in 1 min (Run time: 16 min); Flow rate 0.35 ml/min; Temperature: 25° C.; PDA Scan: 210-400 nm.

### UPLC-MS Method-2

Instrument: Waters Acquity UPLC-MS SQD 3100; Column: Acquity UPLC BEH Shield RP 18, 1.7 μm, 2.1×50 mm; Eluent A: 0.05% TFA in Water, Eluent B: Acetonitrile; Gradient: 10% B to 50% B in 2 min, hold for 1 min, 90% B in 0.5 min hold for 1 min, 10% B in 0.1 min (Run time: 6.0 min); Flow rate 0.35 ml/min; Temperature: 25° C.; PDA Scan: 210-400 nm.

### UPLC-MS Method-3

Instrument: Waters Acquity UPLC-MS SQD 3100; Column: Acquity UPLC BEH Shield RP 18, 1.7 μm, 2.1×50 mm; Eluent A: 0.05% TFA in Water, Eluent B: Acetonitrile; Gradient: 10% B to 50% B in 4.5 min, hold for 3 min, 50% B to 10% B in 0.5 min (Run time: 10.0 min); Flow rate 0.35 ml/min; Temperature: 25° C.; PDA Scan: 210-400 nm.

### UPLC-MS Method-4

Instrument: Waters Acquity UPLC-MS SQD 3100; Column: Acquity UPLC BEH Shield RP 18, 1.7 μm, 2.1×50 mm; Eluent A: 0.05% TFA in Water, Eluent B: Acetonitrile; Gradient: 10% B hold for 0.2 min, 10% B to 90% B in 1.8 min, hold for 1.5 min, 90% B to 10% B in 0.1 min (Run time: 6.0 min); Flow rate 0.35 ml/min; Temperature: 25° C.; PDA Scan: 210-400 nm.

### UPLC-MS Method-5

Instrument: Waters Acquity UPLC-MS SQD 3100; Column: Acquity UPLC BEH Shield RP 18, 1.7 μm, 2.1×50 mm; Eluent A: 0.05% TFA in Water, Eluent B: Acetonitrile; Gradient: 2% B hold for 0.5 min, 2% B to 20% B in 3 min, hold for 1.5 min, 20% B to 2% B in 0.1 min (Run time: 6.0 min); Flow rate 0.35 ml/min; Temperature: 25° C.; PDA Scan: 210-400 nm.

### UPLC-MS Method-6

Instrument: Waters Acquity UPLC-MS SQD 3100; Column: Acquity UPLC BEH Shield RP 18, 1.7 μm, 2.1×50 mm; Eluent A: 0.05% TFA in Water, Eluent B: Acetonitrile; Gradient: 10% B to 50% B in 4.5 min, hold for 3 min, 50% B to 10% B in 0.5 min (Run time: 10.0 min); Flow rate 0.35 ml/min; Temperature: 25° C.; PDA Scan: 210-400 nm.

### UPLC-MS Method-7

Instrument: Waters Acquity UPLC-MS SQD 3100; Column: Acquity UPLC BEH Shield RP 18, 1.7 μm, 2.1×50 mm; Eluent A: 0.05% TFA in Water, Eluent B: Acetonitrile; Gradient: 10% B to 90% B in 4.5 min, hold for 3 min, 90% B to 10% B in 0.5 min (Run time: 10.0 min); Flow rate 0.35 ml/min; Temperature: 25° C.; PDA Scan: 210-400 nm.

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be incorporated in tablet in an amount ranging from about 1 mg to about 1000 mg.

Compositions comprising a compound provided herein are also described. In one variation, the composition comprises a compound or salt thereof and a pharmaceutically acceptable carrier or excipient. In another variation, a composition of substantially pure compound is provided.

### Methods of Use

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease in an individual comprising administering an effective amount of a compounds of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)) or any embodiment, variation or aspect thereof or the present compounds or the compounds detailed or described herein) or a pharmaceutically acceptable salt thereof, to the individual. Further provided herein is a method of treating a proliferative disease in an individual, comprising administering an effective amount of the compounds of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)) or a pharmaceutically acceptable salt thereof, to the individual. Also provided herein is a method of treating cancer in an individual comprising administering an effective amount of the compounds of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)) or a pharmaceutically acceptable salt thereof, to the individual. In some embodiments, the compound is administered to the individual according to a dosage and/or method of administration described herein.

Another aspect of the invention relates to a method of treating a disease or disorder associated with mutant isocitrate dehydrogenase. The method involves administering to a patient in need of a treatment for diseases or disorders associated with mutant isocitrate dehydrogenase an effective amount of the compositions and compounds of the present invention (collectively, a compound of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)) or a pharmaceutically acceptable salt thereof.

Another aspect of the invention is directed to a method inhibiting mutant isocitrate dehydrogenase. The method involves administering to a patient in need thereof an effective amount of the compositions or compounds of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)) or a pharmaceutically acceptable salt thereof.

Examples of a mutant IDH protein having a neomorphic activity are mutant IDH1 and mutant IDH2. A neomorphic activity associated with mutant IDH1 and mutant IDH2 is the ability to produce 2-hydroxyglutarate (2-HG neomorphic activity), specifically R-2-HG (R-2-HG neomorphic activity). Mutations in IDH 1 associated with 2-HG neomorphic activity, specifically R-2-HG neomorphic activity, include mutations at residues 97, 100, and 132, e.g. G97D, R100Q, R132H, R132C, R132S, R132G, R132L, and R132V. Mutations in IDH2 associated with 2-HG neoactivity, specifically R-2-HG neomorphic activity, include mutations at residues 140 and 172, e.g. R140Q, R140G, R172K, R172M, R172S, R172G, and R172W.

Another aspect of the invention relates to method of reducing alpha-ketoglutarate. The method comprises administering to a patient in need thereof an effective amount of the compositions or compounds of the present invention of formula (IA), (I), (Ia-1) to (Ia-14), (Ib-1) to (Ib-11), (II), (IIa-1) to (IIa-8), (III), (IIIa-1) to (IIIa-8), (IV), (IVa-1) to (IVa-7), (V) or (VI)) or a pharmaceutically acceptable salt thereof.

One therapeutic use of the compounds or compositions of the present invention which inhibit mt-IDH is to provide treatment to patients or subjects suffering from cell proliferative diseases and cancers including, without limitation, glioma, glioblastoma multiforme, paraganglioma, supratentorial primordial neuroectodermal tumors, acute myeloid leukemia (AML), prostate cancer, thyroid cancer, colon cancer, chondrosarcoma, cholangiocarcinoma, peripheral T-cell lymphoma, melanoma, intrahepatic cholangiocarcinoma (IHCC), myelodysplastic syndrome (MDS), myeloproliferative disease (MPD), and other solid tumors. Targeted treatments for these cancers and cell proliferative diseases are not currently available to patients suffering from these conditions. Therefore, there is a need for new therapeutic agents selective to these conditions.

Another therapeutic use of the compounds or compositions of the present invention which inhibit mt-IDH is to provide treatment to patients or subjects suffering from cell proliferative diseases and cancers including sarcomas and carcinomas, In some embodiments, examples such as sarcomas and carcinomas are cancer that may be treated as solid tumors. In some embodiments, examples such as leukemia are the cancer that may be treated as liquid tumors. Present invention may treat different types of cancers that include, but are not limited to, adrenocortical cancer, bladder cancer, brain tumors, breast cancer, prostate cancer, colorectal cancer, colon cancer, endometrial cancer, gallbladder cancer, gastric cancer, head and neck cancer, hematopoietic cancer, kidney cancer, leukemia, oral cancer, uterine carcinoma, Hodgkin lympoma, liver cancer, lung cancer, pancreatic cancer, prostate cancer, ovarian cancer, sarcoma, skin cancer and thyroid cancer. In some embodiments, the breast cancer is classified as carcinoma of breast (ER negative or ER positive), mammary adenocarcinoma, primary breast ductal carcinoma, mammary ductal carcinoma (ER positive, ER negative or HER2 positive), triple negative breast cancer (TNBC), HER2 positive breast cancer or luminal breast cancer. In some embodiments, the breast cancer is unclassified. In some embodiments, a basal-like TNBC, an immunomodulatory TNBC, mesenchymal TNBC (mesenchymal or mesenchymal stem-like) or a luminal androgen receptor TNBC are triple negative breast. In some embodiments, prostate adenocarcinoma is prostate cancer. In some embodiments, the ovary adenocarcinoma is ovarian cancer. In some embodiments, lung carcinoma, adenocarcinoma, non-small lung carcinoma, mucoepidermoid, anaplastic large cell are lung cancer. In some embodiments, the lung cancer is unclassified. In some embodiments, the colon adenocarcinomas, colon carcinoma, metastatic colorectal cancer, colon adenocarcinoma from a metastatic site lymph node are colon cancer. In some embodiments astrocytoma, glioblastoma, meduloblastoma, neuroblastoma or meningioma is brain tumor. In some embodiments, stomach cancer is gastric cancer. In some embodiments, cholangiocarcinoma or hepatoblastoma, hepatocellular carcinoma are liver cancers. In some embodiments, liver cancer is derived from hepatitis B virus. In some embodiments, liver cancer is virus negative. In some embodiments, medullary thyroid cancer or follicular thyroid cancer, papillary thyroid carcinomas are classified as thyroid cancer. In some embodiments, uterine papillary serous carcinoma or uterine clear cell carcinoma, high grade endometroid cancer are endometrial cancer. In some embodiments, gallbladder adenocarcinoma or squamous cell gallbladder carcinoma are gallbladder cancer. In some embodiments, renal cell carcinoma or urothelial cell carcinoma are classified as kidney cancer. In some embodiments, adrenal cortical carcinoma adrenocortical is cancer. In some embodiments, fibrosarcoma or Ewing's sarcoma, osteosarcoma, rhabdomiosarcoma and synovial sarcoma are classified as sarcoma. In some embodiments, basal cell carcinoma, melanoma or squamous carcinoma are classified as skin cancer. In some embodiments, cancer of the trachea, laryngeal cancer, nasopharyngeal cancer and oropharyngeal cancer are classified as head and neck cancer. In some embodiments, acute lymphoblastic leukemia, acute promyelocytic leukemia, chronic myelogenous leukemia, chronic lymphocytic leukemia, mantle cell lymphoma or multiple myeloma are classified as leukemia.

The disclosed compounds of the invention can be administered in effective amounts to treat or prevent a disorder and/or prevent the development thereof in subjects.

### Combination Therapy

The compound of the present invention may be administered either simultaneously with, or before or after, one or more other therapeutic agents. The compound of the present invention may be administered separately, by the same or different route of administration, or together in the same pharmaceutical composition as the other agents.

In some embodiments, the methods described herein comprise the additional step of co-administering to a subject in need thereof a second therapy e.g., an additional cancer therapeutic agent or an additional cancer treatment. In one embodiment, the other therapeutic agent is selected from: vascular endothelial growth factor (VEGXF) receptor inhibitors, topoisomerase II inhibitors, smoothened inhibitors, alkylating agents, chemotherapy agents, anti-tumor antibiotics, anti-metabolites, retinoids, immunomodulatory or agents including but not limited to anti-cancer vaccines, CTLA-4, LAG-3 and PD-1 antagonists.

Examples of alkylating agents, include but are not limited to, temozolomide, dactinomycin, melphalan, altretamine, carmustine, bendamustine, busulfan, carboplatin, lomustine, cisplatin, chlorambucil, cyclophosphamide, dacarbazine, altretamine, ifosfamide, procarbazine, meclorethamine, streptozocin, thiotepa.

In some embodiments, the additional cancer therapeutic agent is a chemotherapy agent. Examples of chemotherapeutic agents used in cancer therapy include, for example, animetabolites (e.g., folic acid, purine, and pyrimidine derivatives), alkylating agents (e.g., nitrogen mustards, nitrosoureas, platinum, alkyl sulfonates, hydrazines, triazenes, aziridines, spindle poison, cytotoxic agents, topoisomerase inhibitors and others), and hypomethylating agents (e.g., decitabine (5-azadeoxycytidine), zebularine, isothiocyanates, azacitidine (5-azacytidine), 5-flouro-2′-deoxycytidine, 5,6-dihydro-5-azacytidine and others). Exemplary agents include Aclarubicin, Actinomycin, Alitretinoin, Altretamine, Aminopterin, Aminolevulinic acid, Amrubicin, Amsacrine, Anagrelide, Arsenic trioxide, Asparaginase, Atrasentan, Belotecan, Bexarotene, bendamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaportin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

In some embodiments, the additional cancer therapeutic agent is a PARP inhibitors such as Olaparib, Rucaparib, Niraparib and Talazoparib.

Other possible additional therapeutic modalities include tyrosine kinase inhibitors, cyclin-dependent kinase inhibitors, gene therapy, hormonal therapy, peptide and dendritic cell vaccines, synthetic chlorotoxins, and radiolabeled drugs and antibodies.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a salt thereof may be administered to an individual via various routes, including, e.g., intravenous, intramuscular, subcutaneous, oral and transdermal. A compound provided herein can be administered frequently at low doses, known as ‘metronomic therapy,’ or as part of a maintenance therapy using compound alone or in combination with one or more additional drugs. Metronomic therapy or maintenance therapy can comprise administration of a compound provided herein in cycles. Metronomic therapy or maintenance therapy can comprise intra-tumoral administration of a compound provided herein.

In one aspect, the invention provides a method of treating cancer in an individual by parenterally administering to the individual (e.g., a human) an effective amount of a compound or salt thereof. In some embodiments, the route of administration is intravenous, intra-arterial, intramuscular, or subcutaneous. In some embodiments, the route of administration is oral. In still other embodiments, the route of administration is transdermal.

The invention also provides compositions (including pharmaceutical compositions) as described herein for the use in treating, preventing, and/or delaying the onset and/or development of cancer and other methods described herein. In certain embodiments, the composition comprises a pharmaceutical formulation which is present in a unit dosage form

Also provided are articles of manufacture comprising a compound of the disclosure or a salt thereof, composition, and unit dosages described herein in suitable packaging for use in the methods described herein. Suitable packaging is known in the art and includes, for example, vials, vessels, ampules, bottles, jars, flexible packaging and the like. An article of manufacture may further be sterilized and/or sealed.

### Kits:

The present disclosure further provides kits for carrying out the methods of the invention, which comprises one or more compounds described herein or a composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual

The invention can be further understood by reference to the following examples, which are provided by way of illustration and are not meant to be limiting.

Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as defined by the claims.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

### EXAMPLES

### Example-1: Synthesis of (S)-3-(4-((S)-1-(5-(4-fluoro-3-methylphenyl)pyrimidin-2-yl)ethylamino)-1,3,5-triazin-2-yl)-4-isopropyloxazolidin-2-one (Compound 1.1); and (S)-3-(4-((R)-1-(5-(4-fluoro-3-methylphenyl)pyrimidin-2-yl)pyrimidin-2-yl)ethylamino)-1,3,5-triazin-2-yl)-4-isopropyloxazolidin-2-one (Compound 1.2)

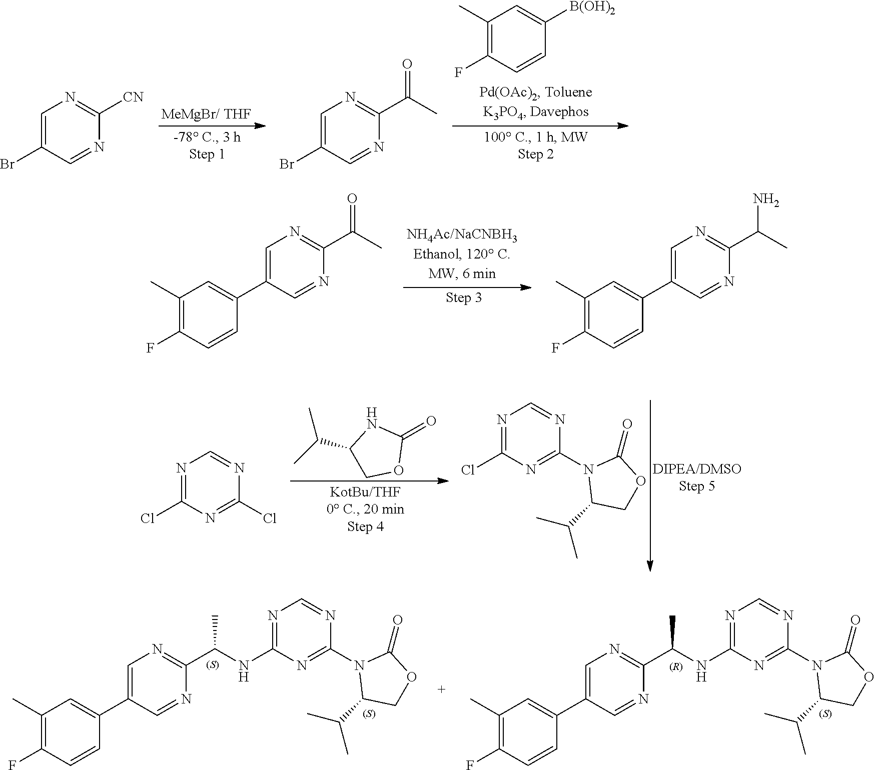

### Step-1: Synthesis of 1-(5-bromopyrimidin-2-yl)ethan-1-one

To a stirred solution of 5-bromopyrimidine-2-carbonitrile (3.0 g, 16.30 mmol, 1.0 eq.) in diethyl ether (60 mL) was added methyl magnesium bromide (19.0 ml, 57.06 mmol, 3.5 eq.) at −78° C. over a period of 15 minutes. The reaction mixture was stirred at −78° C. for 3 h. After completion of reaction, the reaction mixture was quenched with saturated NH₄Cl solution and extracted with EtOAc (2×250 mL). The combined organic layers were washed with water (30 mL) and brine solution (30 mL), dried over anhydrous Na₂SO₄, filtered and concentrated under reduced pressure to afford crude product, which was purified by normal phase silica-gel chromatography to afford the desired compound (0.730 g, 22.32%) LCMS: 201.1/203.1 [M+2]⁺

### Step-2: Synthesis of 1-(5-(4-fluoro-3-methylphenyl)pyrimidin-2-yl)ethan-1-one

To a stirred solution of 1-(5-bromopyrimidin-2-yl)ethan-1-one (730 mg, 3.63 mmol, 1.0 eq.) and (4-fluoro-3-methylphenyl)boronic acid (1.18 g, 7.26 mmol, 2.0 eq.) in toluene (9 mL) was added K₃PO₄ (2.31 g, 10.89 mmol, 3.0 eq.) at RT. The resulting mixture was purged with nitrogen for 10 min followed by addition of Pd(OAc)₂ (41 mg, 0.18 mmol, 0.05 eq.) and Davephos (143 mg, 0.36 mmol, 0.10 eq.), again purged with nitrogen for 10 min. The reaction mixture was heated at 100° C. for 1 h under microwave irradiation. The progress of reaction was monitored by LCMS. The reaction mixture was filtered through celite; the residue was washed with EtOAc (10 mL). The filtrate was concentrated and purified by normal phase silica-gel chromatography to afford the desired compound (0.2 g, 23.92%) LCMS: 231.1 [M+1]⁺

### Step-3: Synthesis of 1-(5-(4-fluoro-3-methylphenyl)pyrimidin-2-yl)ethan-1-amine

1-(5-(4-fluoro-3-methylphenyl)pyrimidin-2-yl)ethan-1-one (115 mg, 0.49 mmol, 1.0 eq.), NH₄OAc (0.385 g, 4.99 mmol, 10.0 eq.), and NaBH₃CN (0.022 g, 0.34 mmol, 0.7 eq.) were taken up in 6 mL of EtOH, and heated at 120° C. for 6 min in a microwave apparatus. The mixture was concentrated under reduced pressure to remove the EtOH. Crude product obtained was basified with 6N NaOH until pH was 10. The reaction mixture was extracted with EtOAc (3×25 mL). The combined organic layers were washed with brine (25 mL), dried over Na₂SO₄ and concentrated under reduced pressure to give, title compound (0.1 g) crude, which was carried forward without any further purification LCMS: 232.2 [M+1]⁺

### Step-4: Synthesis of (S)-3-(4-chloro-1,3,5-triazin-2-yl)-4-isopropyloxazolidin-2-one

To a stirred solution of (S)-4-isopropyloxazolidin-2-one (0.2 g, 1.54 mmol, 1 eq.) and 2,4-dichloro-1,3,5-triazine (0.278 g, 1.85 mmol, 1.2 eq.) in (6 mL) of THF was added KOtBu (0.518 g, 4.62 mmol, 3.0 eq.) at 0° C. and allowed to stir at 0° C. for 20 min. After completion of reaction, the reaction mixture was diluted with water, extracted with EtOAc (3×25 mL). The combined organic layers were washed with brine (25 mL) and dried over anhydrous Na₂SO₄, filtered and concentrated under reduced pressure to afford the crude product, which was purified by normal phase silica-gel chromatography to afford the title compound, (0.15 g, 46.34%) LCMS: 243.1 [M+2]⁺

### Step-5: Synthesis of (4S)-3-(4-{[(1S)-1-[5-(4-fluoro-3-methylphenyl)pyrimidin-2-yl]ethyl]amino}-1,3,5-triazin-2-yl)-4-(propan-2-yl)-1,3-oxazolidin-2-one and (4S)-3-(4-{[(1R)-1-[5-(4-fluoro-3-methylphenyl)pyrimidin-2-yl]ethyl]amino}-1,3,5-triazin-2-yl)-4-(propan-2-yl)-1,3-oxazolidin-2-one

To a stirred solution of (S)-3-(4-chloro-1,3,5-triazin-2-yl)-4-isopropyloxazolidin-2-one (0.07 g, 0.28 mmol, 1 eq.) and 1-(5-(4-fluoro-3-methylphenyl)pyrimidin-2-yl)ethan-1-amine (0.1 g, 0.43 mmol, 1.5 eq.) in (6 mL) of DMSO was added DIPEA (0.14 mL, 0.86 mmol, 3.0 eq.) at RT. The reaction mixture was heated at 120° C. for 1 h under microwave irradiation. After completion of reaction, the reaction mixture was diluted with water, extracted with EtOAc (3×25 mL). The combined organic layers were washed with brine (25 mL) and dried over anhydrous Na₂SO₄, filtered and concentrated under reduced pressure to afford the crude product, which was purified by reverse phase column purification to get the title compounds. Compound 1.1 (0.01 g, 6%), UPLC-MS (Method 2): Rt 3.27, m/z 438.3 [M+1]⁺; ¹H NMR (400 MHz, METHANOL-d4): δ ppm 8.99 (s, 2H) 8.34 (d, J=9.21 Hz, 1H) 7.60 (d, J=7.45 Hz, 1H) 7.53 (br. s., 1H) 7.19 (t, J=8.99 Hz, 1H) 5.37 (d, J=7.02 Hz, 1H) 5.18-5.26 (m, 1H) 4.70-4.75 (m, 1H) 4.51-4.55 (m, 1H) 4.39 (d, J=7.45 Hz, 1H) 4.29-4.34 (m, 1H) 4.20-4.27 (m, 1H) 2.57 (br. s., 1H) 2.36 (s, 3H) 1.59-1.71 (m, 4H) 1.38 (br. s., 1H) 1.29 (br. s., 2H) 0.98 (d, J=7.02 Hz, 1H) 0.89 (d, J=6.58 Hz, 1H) 0.70 (d, J=7.45 Hz, 3H) 0.61 (d, J=7.02 Hz, 3H); Compound 1.2 (0.011 g, 6%), UPLC-MS (Method 2): Rt 3.44, m/z 438.3 [M+1]⁺; ¹HNMR (400 MHz, METHANOL-d4): δ ppm 8.99 (s, 2H) 8.33 (d, J=4.38 Hz, 1H) 7.60 (d, J=6.14 Hz, 1H) 7.53 (br. s., 1H) 7.19 (t, J=9.21 Hz, 1H) 5.37 (d, J=6.14 Hz, 1H) 5.23 (d, J=7.02 Hz, 1H) 4.71 (br. s., 1H) 4.33-4.40 (m, 1H) 4.21-4.31 (m, 1H) 2.59 (br. s., 2H) 2.35 (s, 2H) 1.64 (t, J=6.36 Hz, 3H) 1.29 (br. s., 3H) 0.94-1.05 (m, 3H) 0.89 (t, J=7.45 Hz, 3H).

### Example-2: Synthesis of (S)-3-(4-((S)-1-(5-(4-fluoro-3-methylphenyl)pyridin-2-yl)ethylamino)-1,3,5-triazin-2-yl)-4-isopropyloxazolidin-2-one. (Compound 1.3)

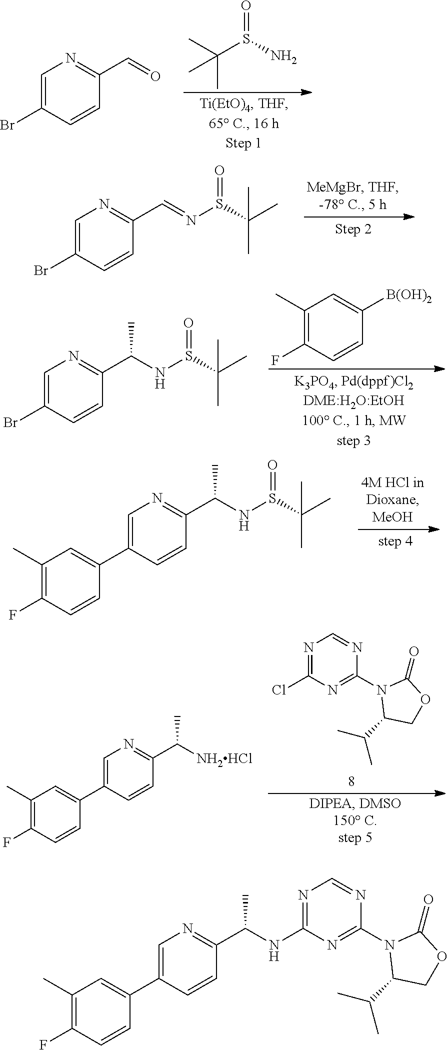

### Step-1: Synthesis of (S,E)-N-((5-bromopyridin-2-yl)methylene)-2-methylpropane-2-sulfinamide

To a stirred solution of 5-bromopicolinaldehyde (5 g, 26.88 mmol, 1.0 eq.) and Ti(EtO)₄ (12.26 g, 53.76 mmol, 2.0 eq.) in dichloromethane (30 mL) was added (S)-2-methylpropane-2-sulfinamide (3.24 g, 26.88 mmol, 1.0 eq.) at RT. The resulting mixture was heated at 65° C. for 16 h. Following this, reaction was allowed to cool to room temperature, filtered through celite pad, the celite pad washed with dichloromethane (30 mL). The combined filtrate dried over anhydrous Na₂SO₄ and concentrated under vacuum to get the solid residue which was purified by normal phase silica-gel column chromatography to get the title compound (6.58 g, 72%) LCMS: 290.9 [M+2]⁺; ¹H NMR (400 MHz, DMSO-d₆) δ ppm 8.91 (d, J=1.96 Hz, 1H) 8.45 (s, 1H) 8.26 (dd, J=8.31, 1.96 Hz, 1H) 8.03 (d, J=8.31 Hz, 1H) 1.20 (s, 9H).

### Step-2: Synthesis of (S)—N—((S)-1-(5-bromopyridin-2-yl)ethyl)-2-methylpropane-2-sulfinamide

To a stirred solution of (S,E)-N-((5-bromopyridin-2-yl)methylene)-2-methylpropane-2-sulfinamide (5.87 g, 20.38 mmol, 1.0 eq.) in tetrahydrofuran (100 mL) was added drop wise 3M methylmagnesium bromide (10.19 mL, 30.57 mmol, 1.5 eq.) at −78° C. The resulting mixture was stirred for 5 h at same temperature. The reaction was then quenched by careful addition of saturated NH₄Cl (50 mL). The aqueous layer was separated and extracted with ethyl acetate (3×50 mL). The combined organic layers were dried over Na₂SO₄, filtered and concentrated to give crude solid residue which was purified by normal phase silica-gel column chromatography to get the title compound as semi-solid (5.59 g, 88%). LCMS: 307.0 [M+2]⁺; ¹H NMR (400 MHz, DMSO-d₆) δ ppm 8.62 (d, J=2.45 Hz, 1H) 8.05 (dd, J=8.56, 2.20 Hz, 1H) 7.53 (d, J=8.31 Hz, 1H) 5.77 (d, J=8.31 Hz, 1H) 4.37-4.47 (m, 1H) 1.41 (d, J=6.85 Hz, 3H) 1.12 (s, 9H)

### Step-3: Synthesis of (S)—N—((S)-1-(5-(4-fluoro-3-methylphenyl)pyridin-2-yl)ethyl)-2-methylpropane-2-sulfinamide

To a stirred solution of (S)—N—((S)-1-(5-bromopyridin-2-yl)ethyl)-2-methylpropane-2-sulfinamide (1.0 g, 3.27 mmol, 1.0 eq.) and 4-fluoro-3-methylphenylboronic acid (0.608 g, 3.93 mmol, 1.2 eq.) in dimethoxyethane:H₂O:EtOH (14:6:4 mL) was added K₃PO₄ (1.27 g, 6.55 mmol, 2.0 eq.). The reaction mixture was purged with N₂ for about 15 min and Pd(dppf)Cl₂-DCM complex (0.24 g, 0.1 mol %) was added. Reaction mixture was re-purged with N₂ and heated at 100° C. for 16 h. Following this, reaction was allowed to cool to RT and filtered through celite pad, the celite pad washed with ethyl acetate and water. The aqueous layer was separated extracted using ethyl acetate (3×10 mL). The combined organic layers were washed with brine (30 mL), dried over anhydrous Na₂SO₄, filtered and concentrated under vacuum to get the solid residue which was carried forward without any further purification (0.93 g, 85%). LCMS: 335.3 [M+1]⁺.

### Step-4: Synthesis of (S)-1-(5-(4-fluoro-3-methylphenyl)pyridin-2-yl)ethanamine hydrochloride

To a stirred solution of (S)—N—((S)-1-(5-(4-fluoro-3-methylphenyl)pyridin-2-yl)ethyl)-2-methylpropane-2-sulfinamide (0.43 g, 1.28 mmol, 1.0 eq.) in methanol (5 mL) was added 4N HCl in dioxane (1.6 mL, 6.43 mmol, 5.0 eq.) at RT. The resulting mixture was stirred for 30 min. Following this, the reaction mixture was evaporated under reduced pressure to get solid residue. The obtained solid was washed with diethyl ether, dried under vacuum to get the title compound (0.28 g, 96%). LCMS: 231.1 [M+1]⁺; ¹H NMR (400 MHz, DMSO-d₆) δ ppm 8.91 (d, J=1.96 Hz, 1H) 8.49 (br. s., 3H) 8.17 (dd, J=8.31, 2.45 Hz, 1H) 7.71 (d, J=7.34 Hz, 1H) 7.59-7.65 (m, 2H) 7.29 (t, J=9.29 Hz, 1H) 4.55-4.60 (m, 1H) 2.29-2.36 (m, 3H) 1.53 (d, J=6.36 Hz, 3H).

### Step-5: Synthesis of (S)-3-(4-((S)-1-(5-(4-fluoro-3-methylphenyl)pyridin-2-yl)ethylamino)-1,3,5-triazin-2-yl)-4-isopropyloxazolidin-2-one

In a microwave vial charged with (S)-1-(5-(4-fluoro-3-methylphenyl)pyridin-2-yl)ethanamine hydrochloride (0.15 g, 0.65 mmol, 1.0 eq.), (S)-3-(4-chloro-1,3,5-triazin-2-yl)-4-isopropyloxazolidin-2-one (0.158 g, 0.65 mmol, 1.0 eq.) and N,N-Diisopropylethylamine (2.7 mL, 1.95 mmol, 3.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound (0.03 g, 12%). UPLC-MS (Method 4): Rt 2.96, m/z 437.3 [M+1]⁺; ¹H NMR (400 MHz, DMSO-d₆) δ 8.78 (d, J=1.47 Hz, 1H), 8.70 (d, J=7.34 Hz, 1H), 8.55 (d, J=7.83 Hz, 1H), 8.36-8.44 (m, 1H), 7.96-8.04 (m, 1H), 7.62 (d, J=6.36 Hz, 1H), 7.53 (br. s., 1H), 7.45 (d, J=8.31 Hz, 1H), 7.38 (d, J=8.31 Hz, 1H), 7.25 (t, J=9.29 Hz, 1H), 5.19-5.31 (m, 1H), 5.05-5.16 (m, 1H), 4.58 (d, J=7.83 Hz, 1H), 4.41 (d, J=8.31 Hz, 1H), 4.15-4.36 (m, 3H), 2.30 (s, 3H), 1.76 (br. s., 1H), 1.52 (d, J=6.85 Hz, 3H), 0.90 (d, J=6.85 Hz, 1H), 0.79 (d, J=6.85 Hz, 1H), 0.62 (d, J=6.85 Hz, 2H), 0.53 (d, J=7.34 Hz, 2H).

### Example-3: Synthesis of (S)-4-isopropyl-3-(4-((S)-1-(5-(4-(trifluoromethyl)phenyl)pyridin-2-yl)ethylamino)-1,3,5-triazin-2-yl)oxazolidin-2-one. (Compound No. 1.4)

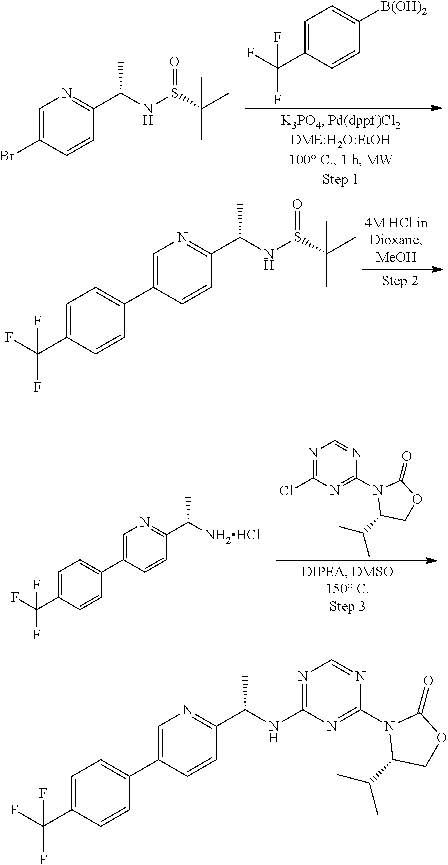

### Step-1: Synthesis of (S)-2-methyl-N—((S)-1-(5-(4-(trifluoromethyl)phenyl)pyridin-2-yl)ethyl)propane-2-sulfinamide

To a stirred solution of (S)—N—((S)-1-(5-bromopyridin-2-yl)ethyl)-2-methylpropane-2-sulfinamide (0.4 g, 1.306 mmol, 1.0 eq.) and 4-(trifluoromethyl)phenylboronic acid (0.289 g, 1.567 mmol, 1.2 eq.) in dimethoxyethane:H₂O:EtOH (7:3:2 mL) was added K₃PO₄ (0.554 g, 2.612 mmol, 2.0 eq.). The reaction mixture was purged with N₂ for about 15 min and Pd(dppf)Cl₂DCM complex (0.105 g, 0.1 mol %) was added. Reaction mixture was re-purged with N₂ and heated at 100° C. for 1 h under microwave irradiation. Following this, reaction was allowed to cool to RT and filtered through celite pad, the celite pad washed with ethyl acetate and water. The aqueous layer was separated extracted using ethyl acetate (3×10 mL). The combined organic layers were washed with brine (10 mL), dried over anhydrous Na₂SO₄, filtered and concentrated under vacuum to get the solid residue which was purified by normal phase silica gel column chromatography to get the title compound (0.4 g, 83%). LCMS: 371.2 [M+1]⁺; ¹H NMR (400 MHz, DMSO-d₆) δ ppm 8.89 (d, J=2.19 Hz, 1H) 8.18 (dd, J=8.33, 2.19 Hz, 1H) 7.97 (m, J=7.89 Hz, 2H) 7.86 (m, J=8.33 Hz, 2H) 7.68 (d, J=7.89 Hz, 1H) 5.82 (d, J=7.45 Hz, 1H) 4.49-4.56 (m, 1H) 1.47 (d, J=7.02 Hz, 3H) 1.15 (s, 9H)

### Step-2: Synthesis of (S)-1-(5-(4-(trifluoromethyl)phenyl)pyridin-2-yl)ethanamine hydrochloride

To a stirred solution of (S)-2-methyl-N—((S)-1-(5-(4-(trifluoromethyl)phenyl)pyridin-2-yl)ethyl)propane-2-sulfinamide (0.3 g, 0.81 mmol, 1.0 eq.) in methanol (5 mL) was added 4N HCl in dioxane (1.0 mL, 4.0 mmol, 5.0 eq.) at RT. The resulting mixture was stirred for 30 min. Following this, the reaction mixture was evaporated under reduced pressure to get solid residue. The obtained solid was washed with diethyl ether, dried under vacuum to get title compound as off-white solid (0.2 g, 95%) LCMS: 267.1 [M+1]⁺; ¹H NMR (400 MHz, DMSO-d₆) δ ppm 9.00 (s, 1H) 8.52 (br. s., 3H) 8.28 (d, J=6.85 Hz, 1H) 8.01 (m, J=7.83 Hz, 2H) 7.88 (m, J=8.31 Hz, 2H) 7.69 (d, J=8.31 Hz, 1H) 4.60 (br. s., 1H) 1.54 (d, J=6.85 Hz, 3H).

### Step-3: Synthesis of (S)-3-(4-((S)-1-(5-(4-fluoro-3-methylphenyl)pyridin-2-yl)ethylamino)-1,3,5-triazin-2-yl)-4-isopropyloxazolidin-2-one

In a microwave vial charged with (S)-3-(4-chloro-1,3,5-triazin-2-yl)-4-isopropyloxazolidin-2-one (0.15 g, 0.56 mmol, 1.0 eq.), (S)-1-(5-(4-(trifluoromethyl)phenyl)pyridin-2-yl)ethanamine hydrochloride (0.158 g, 0.56 mmol, 1.0 eq.) and N, N-Diisopropylethylamine (2.3 mL, 1.69 mmol, 3.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by reversed phase column chromatography to get the title compound as white solid (0.023 g, 8%). UPLC-MS (Method 4): Rt 2.53, m/z 473.4 [M+1]⁺; ¹H NMR (400 MHz, DMSO-d₆) δ ppm 8.89 (d, J=1.96 Hz, 1H) 8.74 (d, J=6.85 Hz, 1H) 8.59 (d, J=8.31 Hz, 1H) 8.37-8.43 (m, 1H) 8.09-8.16 (m, 1H) 7.91-7.97 (m, 2H) 7.83-7.89 (m, 2H) 7.45 (d, J=8.31 Hz, 1H) 5.23-5.31 (m, 1H) 5.09-5.17 (m, 1H) 4.52-4.61 (m, 1H) 4.38-4.45 (m, 1H) 4.16-4.35 (m, 3H) 1.72 (dd, J=6.85, 3.42 Hz, 1H) 1.53 (d, J=7.34 Hz, 3H) 0.90 (d, J=6.85 Hz, 1H) 0.79 (d, J=6.85 Hz, 1H) 0.62 (d, J=6.85 Hz, 2H) 0.52 (d, J=6.85 Hz, 2H).

### Example-4: Synthesis of (S)-4-isopropyl-3-(4-((S)-1-(1-(4-(trifluoromethyl)phenyl)-1H-imidazol-4-yl)ethylamino)-1,3,5-triazin-2-yl)oxazolidin-2-one. (Compound 1.5)

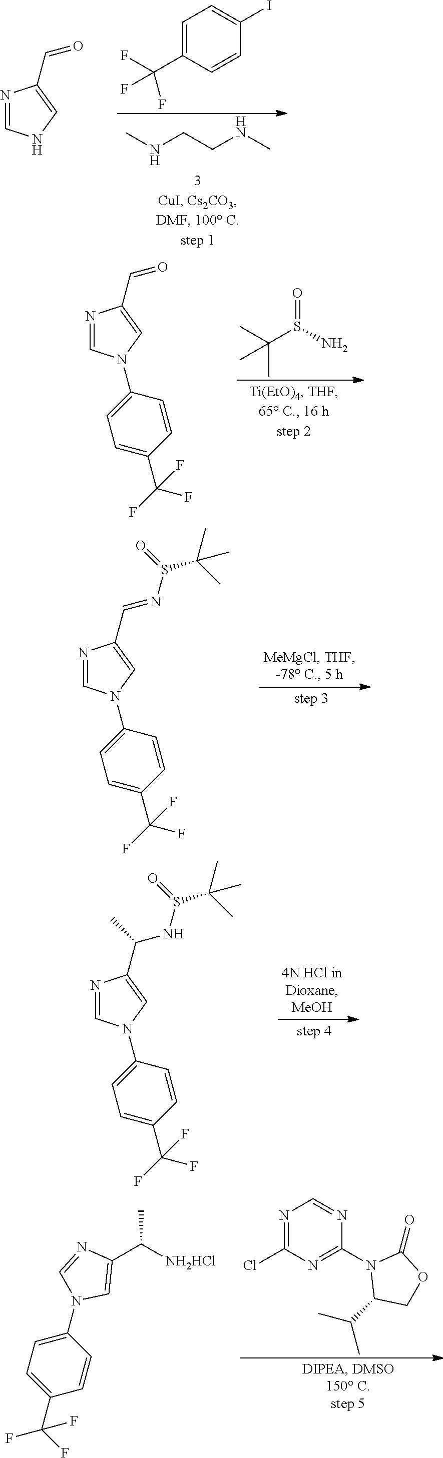

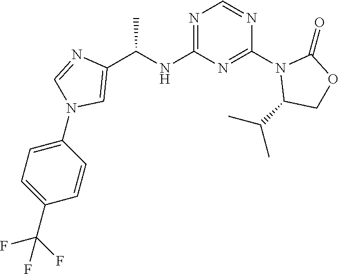 [SMILES: CC(C)[C@H]1COC(=O)N1c1ncnc(N[C@@H](C)c2cn(-c3ccc(C(F)(F)F)cc3)cn2)n1]

### Step-1: Synthesis of 1-(4-(trifluoromethyl)phenyl)-1H-imidazole-4-carbaldehyde

To a stirred solution of 1H-imidazole-4-carbaldehyde (5 g, 52.06 mmol, 1.0 eq.) and 1-Iodo-4-(trifluoromethyl)benzene (9.35 mL, 62.48 mmol, 1.2 eq.) in DMF (50 mL) was added 1,2-Dimethylethylenediamine (1.2 mL, 10.41 mmol, 0.2 eq.) and Cs₂CO₃ (33.9 g, 104.12 mmol, 2 eq.). The reaction mixture was purged with N₂ for about 10 min and CuI (0.989 g, 5.2 mmol, 0.1 eq.) was added. Reaction mixture was re-purged with N₂ and heated at 100° C. for 16 h. Following this, reaction was allowed to cool to RT and filtered through celite pad, the celite pad washed with ethyl acetate and water. The aqueous layer was separated extracted using ethyl acetate (3×50 mL). The combined organic layers were washed with brine (150 mL), dried over anhydrous Na₂SO₄, filtered and concentrated under vacuum to get the solid residue which was purified by was purified by normal phase silica-gel column chromatography to get the title compound as off-white solid (2.5 g, 20%). ¹H NMR (400 MHz, DMSO-d₆) δ ppm 9.84 (s, 1H) 8.77 (d, J=0.98 Hz, 1H) 8.62 (d, J=0.98 Hz, 1H) 8.03 (m, J=8.80 Hz, 2H) 7.95 (m, J=8.31 Hz, 2H).

### Step-2: Synthesis of (S,E)-2-methyl-N-((1-(4-(trifluoromethyl)phenyl)-1H-imidazol-4-yl)methylene)propane-2-sulfinamide

To a stirred solution of 1-(4-(trifluoromethyl)phenyl)-1H-imidazole-4-carbaldehyde (2.5 g, 10.41 mmol, 1.0 eq.) and Ti(EtO)₄ (4.74 g, 20.82 mmol, 2.0 eq.) in dichloromethane (20 mL) was added (S)-2-methylpropane-2-sulfinamide (1.51 g, 12.49 mmol, 1.2 eq.) at RT. The resulting mixture was heated at 65° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound as white solid (2.2 g, 61%). ¹H NMR (400 MHz, DMSO-d₆) δ ppm 8.67 (s, 1H) 8.61 (s, 1H) 8.42 (s, 1H) 8.01 (m, J=8.31 Hz, 2H) 7.95 (m, J=8.31 Hz, 2H) 1.17 (s, 9H)

### Step-3: Synthesis of (S)-2-methyl-N—((S)-1-(1-(4-(trifluoromethyl)phenyl)-1H-imidazol-4-yl)ethyl)propane-2-sulfinamide

To a stirred solution of (S,E)-2-methyl-N-((1-(4-(trifluoromethyl)phenyl)-1H-imidazol-4-yl)methylene)propane-2 sulfinamide (1.0 g, 1.306 mmol, 1.0 eq.) in tetrahydrofuran (50 mL) was added drop wise 3 molar methylmagnesium bromide (1.45 mL, 5.2 mmol, 1.5 eq.) at −78° C. The resulting mixture was stirred for 5 h at same temperature. The reaction was then quenched by careful addition of saturated NH₄Cl (10 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound as semi solid (0.8 g 76%). LCMS: 360.2 [M+1]⁺; ¹H NMR (400 MHz, DMSO-d₆) δ ppm 8.37 (s, 1H) 7.85-7.92 (m, 4H) 7.74 (s, 1H) 5.25 (d, J=5.87 Hz, 1H) 4.32-4.39 (m, 1H) 1.47 (d, J=6.85 Hz, 3H) 1.14 (s, 9H).

### Step-4: Synthesis of (S)-1-(1-(4-(trifluoromethyl)phenyl)-1H-imidazol-4-yl)ethanamine hydrochloride

To a stirred solution of (S)-2-methyl-N—((S)-1-(1-(4-(trifluoromethyl)phenyl)-1H-imidazol-4-yl)ethyl)propane-2-sulfinamide (0.8 g, 2.27 mmol, 1.0 eq.) in methanol (10 mL) was added 4N HCl in dioxane (2.8 mL, 11.13 mmol, 5.0 eq.) at RT. The resulting mixture was stirred for 30 min. Following this, the reaction mixture was evaporated under reduced pressure to get (0.6 g) crude which was carried forward without any further purification. LCMS: 256.0 [M+1]⁺; ¹H NMR (400 MHz, DMSO-d₆) δ ppm 8.71 (s, 1H) 8.42 (br. s., 2H) 8.07 (s, 1H) 7.86-8.02 (m, 4H) 4.41-4.46 (m, 2H) 1.57 (d, J=6.85 Hz, 3H).

### Step-5: Synthesis of (S)-4-isopropyl-3-(4-((S)-1-(1-(4-(trifluoromethyl)phenyl)-1H-imidazol-4-yl)ethylamino)-1,3,5-triazin-2-yl)oxazolidin-2-one

In a microwave vial charged with (S)-1-(1-(4-(trifluoromethyl)phenyl)-1H-imidazol-4-yl)ethanamine hydrochloride (0.15 g, 0.58 mmol, 1.0 eq.), (S)-3-(4-chloro-1,3,5-triazin-2-yl)-4-isopropyloxazolidin-2-one (0.142 g, 0.58 mmol, 1.0 eq.) and N, N-Diisopropylethylamine (0.31 mL, 1.76 mmol, 3.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by reversed phase column chromatography to get the title compound as white solid (0.015 g, 6%). UPLC-MS (Method 2): Rt 2.71, m/z 462.4 [M+1]⁺; ¹H NMR (400 MHz, DMSO-d₆) δ ppm 8.37-8.41 (m, 1H) 8.33-8.36 (m, 1H) 8.25 (d, J=8.80 Hz, 1H) 7.84-7.92 (m, 5H) 7.68-7.71 (m, 1H) 5.24 (br. s., 1H) 5.06-5.13 (m, 1H) 4.50-4.58 (m, 2H) 4.22-4.36 (m, 3H) 1.49-1.54 (m, 3H) 1.23 (s, 1H) 0.89 (d, J=6.85 Hz, 1H) 0.79 (d, J=6.85 Hz, 1H) 0.73 (d, J=6.85 Hz, 2H) 0.68 (d, J=6.85 Hz, 2H).

### Example-5: Synthesis of (S)-3-(4-((R)-1-(3,4-dichlorophenyl)ethylamino)-1,3,5-triazin-2-yl)-4-isopropyloxazolidin-2-one (Compound 1.6); and (S)-3-(4-((S)-1-(3,4-dichlorophenyl)ethylamino)-1,3,5-triazin-2-yl)-4-isopropyloxazolidin-2-one (Compound 1.7)

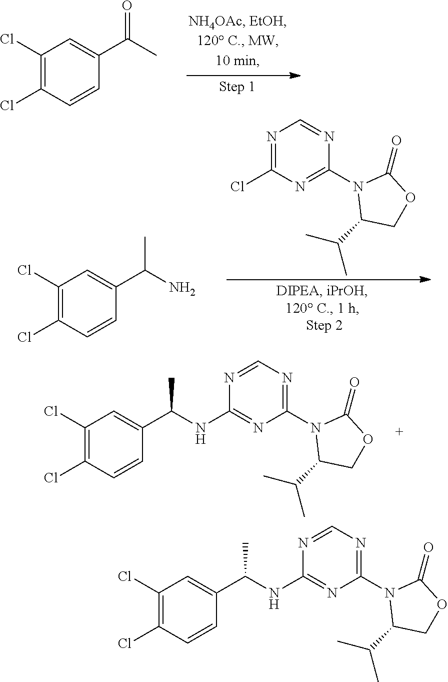

### Step-1: Synthesis of 1-(3,4-dichlorophenyl)ethanamine

In a microwave vial charged with 1-(3,4-dichlorophenyl)ethanone (0.5 g, 2.65 mmol, 1.0 eq.), Ammonium acetate (2.05 g, 26.59 mmol, 10.0 eq.) and sodium cyanoborohydride (0.116 g, 1.85 mmol, 0.7 eq.), in EtOH (10 mL). The resulting mixture was heated at 120° C. for 10 min. Following this, the reaction mixture was allowed to cool to RT, basified with 6N NaOH until pH˜10 and extracted with EtOAc (3×15 mL). The combined organic layers were washed with brine (25 mL), dried over Na₂SO₄ and concentrated under reduced pressure to get the title compound as half white solid which was carried forward without any further purification (0.4 g crude). LCMS: 189.9 [M+1]⁺

### Step-2: Synthesis of (S)-3-(4-((R)-1-(3,4-dichlorophenyl)ethylamino)-1,3,5-triazin-2-yl)-4-isopropyloxazolidin-2-one (Compound 1.6) and (S)-3-(4-((S)-1-(3,4-dichlorophenyl)ethylamino)-1,3,5-triazin-2-yl)-4-isopropyloxazolidin-2-one (Compound 1.7)

In a microwave vial charged with 1-(3,4-dichlorophenyl)ethanamine (0.25 g, 1.32 mmol, 1.0 eq.), (S)-3-(4-chloro-1,3,5-triazin-2-yl)-4-isopropyloxazolidin-2-one (0.32 g, 1.32 mmol, 1.0 eq.) and N,N-Diisopropylethylamine (0.71 mL, 3.96 mmol, 3.0 eq.), in isopropyl alcohol (5 mL). The resulting mixture was heated at 120° C. for 60 min. Following this, the reaction mixture was allowed to cool to RT concentrated under vacuum diluted with water (10 mL) and extracted using ethyl acetate (3×10 mL). The combined organic layers were washed with brine (10 mL), dried over anhydrous Na₂SO₄, filtered and evaporated under reduced pressure to get solid residue. The solid residue was purified by reverse phase column chromatography to get the title compounds. Compound 1.6 (0.005 g, 1%), UPLC-MS (Method 4): Rt 2.71, m/z 396.3 [M+1]⁺; ¹H NMR (400 MHz, DMSO-d₆) δ ppm 8.68 (d, J=7.89 Hz, 1H) 8.59 (d, J=7.89 Hz, 1H) 8.37 (s, 1H) 7.67 (d, J=1.75 Hz, 1H) 7.59 (dd, J=8.33, 2.19 Hz, 1H) 7.36 (d, J=8.33 Hz, 1H) 5.13-5.20 (m, 1H) 4.97-5.04 (m, 1H) 4.51-4.59 (m, 1H) 4.27-4.37 (m, 3H) 1.42-1.49 (m, 3H) 1.23 (s, 1H) 0.85-0.93 (m, 3H) 0.74-0.82 (m, 3H); and Compound 1.7 (0.006, 2% g). UPLC-MS (Method 4): Rt 2.69, m/z 396.3 [M+1]⁺; ¹H NMR (400 MHz, DMSO-d₆) δ ppm 8.71 (d, J=7.45 Hz, 1H) 8.37-8.42 (m, 1H) 7.54-7.66 (m, 2H) 7.28-7.39 (m, 2H) 5.12-5.20 (m, 1H) 5.00-5.10 (m, 1H) 4.57 (d, J=7.89 Hz, 1H) 4.45-4.51 (m, 1H) 4.22-4.36 (m, 3H) 1.44 (d, J=7.02 Hz, 3H) 1.23 (s, 1H) 0.88 (d, J=7.02 Hz, 1H) 0.72-0.79 (m, 3H) 0.62 (d, J=7.02 Hz, 2H)

### Example-6: Synthesis of (S)-4-isopropyl-3-(4-((R)-1-(4-phenoxyphenyl)ethylamino)-1,3,5-triazin-2-yl)oxazolidin-2-one (Compound No. 1.8); and (S)-4-isopropyl-3-(4-((S)-1-(4-phenoxyphenyl)ethylamino)-1,3,5-triazin-2-yl)oxazolidin-2-one (Compound No. 1.9)

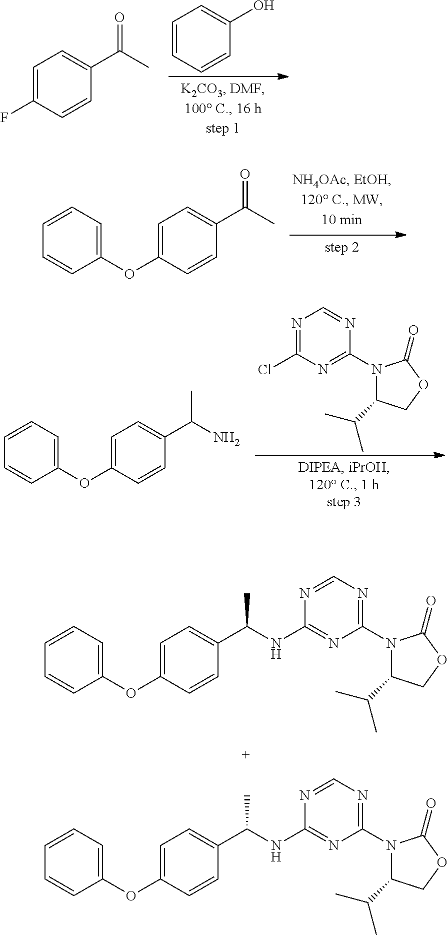

### Step-1: Synthesis of 1-(4-phenoxyphenyl)ethanone

To a stirred solution of 1-(4-fluorophenyl)ethanone (1 g, 7.24 mmol, 1.0 eq.) and phenol (9.35 mL, 62.48 mmol, 1.2 eq.) in DMF (15 mL) was added K₂CO₃ (2.99 g, 21.72 mmol, 3 eq.) and heated at 100° C. for 16 h. Following this, reaction was allowed to cool to RT and filtered through celite pad, the celite pad washed with ethyl acetate and water. The aqueous layer was separated extracted using ethyl acetate (3×20 mL). The combined organic layers were washed with brine (30 mL), dried over anhydrous Na₂SO₄, filtered and concentrated under vacuum to get the solid residue which was carried forward without any further purification (1 g, 65%). LCMS: 213.0 [M+1]⁺

### Step-2: Synthesis of 1-(4-phenoxyphenyl)ethanamine

In a microwave vial charged with 1-(4-phenoxyphenyl)ethanone (1 g, 4.71 mmol, 1.0 eq.), Ammonium acetate (3.63 g, 47.15 mmol, 10.0 eq.) and sodium cyanoborohydride (0.207 g, 3.29 mmol, 0.7 eq.), in EtOH (10 mL). The resulting mixture was heated at 120° C. for 10 min. Following this, the reaction mixture was allowed to cool to RT, basified with 6N NaOH until pH˜10 and extracted with EtOAc (3×15 mL). The combined organic layers were washed with brine (25 mL), dried over Na₂SO₄ and concentrated under reduced pressure to get the title compound as half white solid which was carried forward without any further purification (0.4 g crude) ¹H NMR (400 MHz, DMSO-d₆) δ ppm 7.25-7.44 (m, 5H) 7.11 (t, J=7.24 Hz, 1H) 6.93 (d, J=8.77 Hz, 2H) 6.97 (d, J=8.33 Hz, 2H) 3.98 (q, J=6.58 Hz, 1H) 1.23 (d, J=6.58 Hz, 3H)

### Step-3: Synthesis of (S)-4-isopropyl-3-(4-((R)-1-(4-phenoxyphenyl)ethylamino)-1,3,5-triazin-2-yl)oxazolidin-2-one and (S)-4-isopropyl-3-(4-((S)-1-(4-phenoxyphenyl)ethylamino)-1,3,5-triazin-2-yl)oxazolidin-2-one

In a microwave vial charged with 1-(4-phenoxyphenyl)ethanamine (0.3 g, 1.42 mmol, 1.0 eq.), (S)-3-(4-chloro-1,3,5-triazin-2-yl)-4-isopropyloxazolidin-2-one (0.34 g, 1.42 mmol, 1.0 eq.) and N, N-Diisopropylethylamine (0.75 mL, 4.20 mmol, 3.0 eq.), in isopropyl alcohol (5 mL). The resulting mixture was heated at 120° C. for 60 min. Following this, the reaction mixture was allowed to cool to RT concentrated under vacuum diluted with water (10 mL) and extracted using ethyl acetate (3×10 mL). The combined organic layers were washed with brine (10 mL), dried over anhydrous Na₂SO₄, filtered and evaporated under reduced pressure to get solid residue. The residue was purified by reverse phase column chromatography to get the title compounds. Compound 1.8 (0.034 g, 6%), UPLC-MS (Method 4): Rt 2.71, m/z 420.3 [M+1]⁺; ¹H NMR (400 MHz, DMSO-d₆) δ ppm 8.65 (d, J=7.89 Hz, 1H) 8.55 (d, J=8.33 Hz, 1H) 7.34-7.43 (m, 4H) 7.08-7.16 (m, 1H) 6.92-7.01 (m, 4H) 5.03-5.08 (m, 1H) 4.37 (dd, J=7.24, 3.29 Hz, 1H) 4.25-4.33 (m, 2H) 1.45 (d, J=7.02 Hz, 3H) 1.23 (s, 1H) 0.87-0.92 (m, 3H) 0.81-0.86 (m, 1H) 0.78 (dd, J=7.02, 1.75 Hz, 3H); and Compound 1.9 (0.033 g 6%), UPLC-MS (Method 4): Rt 2.67, m/z 420.3 [M+1]⁺; ¹H NMR (400 MHz, DMSO-d₆) δ ppm 8.69 (d, J=7.89 Hz, 1H) 8.56 (d, J=8.33 Hz, 1H) 8.36 (s, 1H) 7.31-7.42 (m, 6H) 7.09-7.16 (m, 2H) 6.92-7.00 (m, 6H) 5.19 (br. s., 1H) 5.06-5.11 (m, 1H) 4.50-4.59 (m, 2H) 4.23-4.37 (m, 4H) 1.45 (d, J=7.02 Hz, 4H) 1.36 (d, J=7.02 Hz, 1H) 1.23 (s, 2H) 0.88 (d, J=7.02 Hz, 2H) 0.75-0.80 (m, 4H) 0.65 (d, J=7.02 Hz, 3H).

### Example-7: Synthesis of (S)-3-(4-((S)-1-(5-(4-chlorophenyl)pyridin-2-yl)ethylamino)-1,3,5-triazin-2-yl)-4-isopropyloxazolidin-2-one. (Compound No. 1.10)

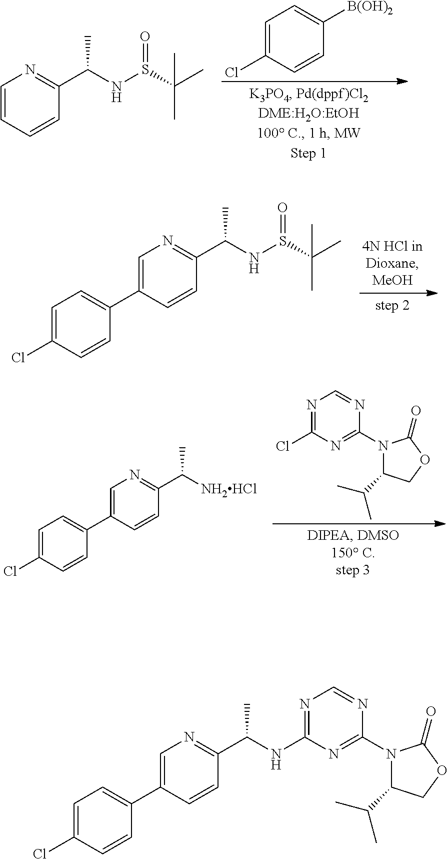

### Step-1: Synthesis of (S)—N—((S)-1-(5-(4-chlorophenyl)pyridin-2-yl)ethyl)-2-methylpropane-2-sulfinamide

To a stirred solution of (S)—N—((S)-1-(5-bromopyridin-2-yl)ethyl)-2-methylpropane-2-sulfinamide (0.4 g, 1.306 mmol, 1.0 eq.) and 4-chlorophenylboronic acid (0.244 g, 1.567 mmol, 1.2 eq.) in dimethoxyethane:H₂O:EtOH (7:3:2 mL) was added K₃PO₄ (0.554 g, 2.612 mmol, 2.0 eq.). The reaction mixture was purged with N₂ for about 15 min and Pd(dppf)Cl₂-DCM complex (0.105 g, 0.1 mol %) was added. Reaction mixture was re-purged with N₂ and heated at 100° C. for 1 h under microwave irradiation. Following this, reaction was allowed to cool to RT and filtered through celite pad, the celite pad washed with ethyl acetate and water. The aqueous layer was separated extracted using ethyl acetate (3×10 mL). The combined organic layers were washed with brine (10 mL), dried over anhydrous Na₂SO₄, filtered and concentrated under vacuum to get the solid residue which was purified by was purified by normal phase silica-gel column chromatography to get the title compound as off white solid (0.4 g, 90%). LCMS: 337.1 [M+1]⁺; ¹H NMR (400 MHz, DMSO-d₆) δ ppm 8.82 (d, J=2.19 Hz, 1H) 8.10 (dd, J=8.33, 2.19 Hz, 1H) 7.77 (m, J=8.33 Hz, 2H) 7.63 (d, J=8.33 Hz, 1H) 7.56 (m, J=8.33 Hz, 2H) 5.79 (d, J=7.45 Hz, 1H) 4.46-4.54 (m, 1H) 1.45 (d, J=7.02 Hz, 3H) 1.14 (s, 9H)

### Step-2: Synthesis of (S)-1-(5-(4-chlorophenyl)pyridin-2-yl)ethanamine hydrochloride

To a stirred solution of (S)—N—((S)-1-(5-(4-chlorophenyl)pyridin-2-yl)ethyl)-2-methylpropane-2-sulfinamide (0.3 g, 0.89 mmol, 1.0 eq.) in methanol (5 mL) was added 4N HCl in dioxane (1.04 mL, 4.4 mmol, 5.0 eq.) at RT. The resulting mixture was stirred for 30 min. Following this, the reaction mixture was evaporated under reduced pressure to get solid residue. The obtained solid was washed with diethyl ether, dried under vacuum to get the title compound as off white solid (0.19 g, 95%). ¹H NMR (400 MHz, DMSO-d₆) δ ppm 8.94 (d, J=1.75 Hz, 1H) 8.57 (br. s., 3H) 8.21 (dd, J=8.33, 2.19 Hz, 1H) 7.81 (m, J=8.77 Hz, 2H) 7.66 (d, J=8.33 Hz, 1H) 7.59 (m, J=8.77 Hz, 2H) 4.55-4.62 (m, 1H) 3.38 (q, J=7.02 Hz, 2H) 1.54 (d, J=6.58 Hz, 3H).

### Step-3: Synthesis of (S)-3-(4-((S)-1-(5-(4-fluoro-3-methylphenyl)pyridin-2-yl)ethylamino)-1,3,5-triazin-2-yl)-4-isopropyloxazolidin-2-one

In a microwave vial charged with (S)-3-(4-chloro-1,3,5-triazin-2-yl)-4-isopropyloxazolidin-2-one (0.15 g, 0.56 mmol, 1.0 eq.), (S)-1-(5-(4-chlorophenyl)pyridin-2-yl)ethanamine hydrochloride (0.129 g, 0.56 mmol, 1.0 eq.) and N, N-Diisopropylethylamine (2.3 mL, 1.69 mmol, 3.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.007 g, 5%), UPLC-MS (Method 2): Rt 3.07, m/z 439.2 [M+1]⁺; ¹H NMR (400 MHz, DMSO-d₆) δ ppm 8.84 (d, J=2.19 Hz, 2H) 8.77 (d, J=7.02 Hz, 1H) 8.61 (d, J=7.89 Hz, 1H) 8.38-8.44 (m, 1H) 8.06-8.13 (m, 2H) 7.71-7.79 (m, 3H) 7.50-7.59 (m, 4H) 7.45 (d, J=8.33 Hz, 1H) 5.25-5.32 (m, 1H) 5.12 (dt, J=14.25, 6.91 Hz, 1H) 4.57 (dd, J=7.89, 3.51 Hz, 1H) 4.39-4.47 (m, 1H) 4.17-4.37 (m, 4H) 1.75 (td, J=6.91, 3.73 Hz, 2H) 1.53 (d, J=7.45 Hz, 5H) 0.90 (d, J=7.02 Hz, 2H) 0.79 (d, J=7.02 Hz, 1H) 0.63 (d, J=7.02 Hz, 3H) 0.53 (d, J=7.02 Hz, 3H).

### Example-8: Synthesis of (S)-4-isopropyl-3-(4-((S)-1-(2′-(trifluoromethyl)-3,4′-bipyridin-6-yl)ethylamino)-1,3,5-triazin-2-yl)oxazolidin-2-one (Compound 1.11)

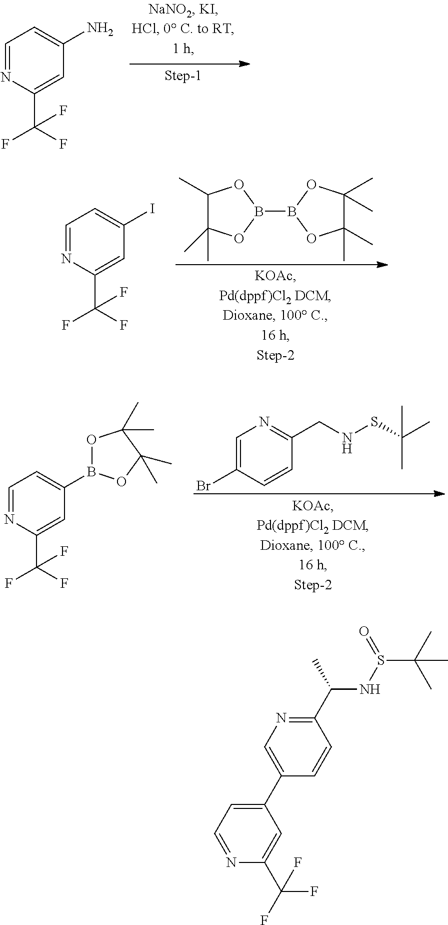

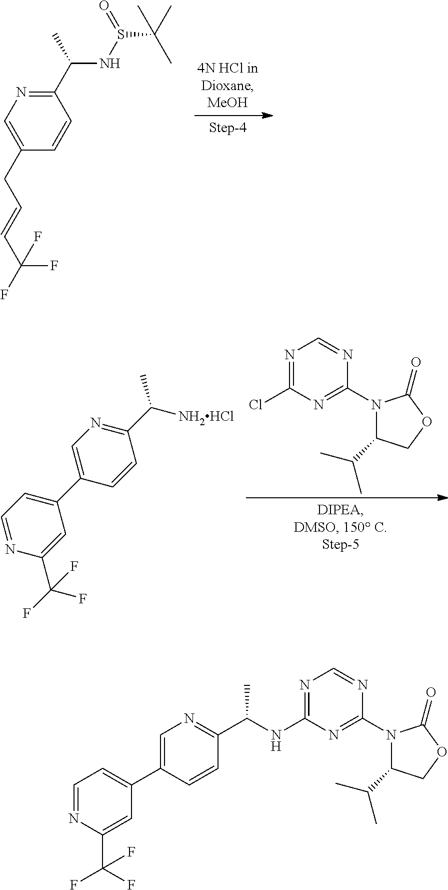

### Step-1: Synthesis of 4-iodo-2-(trifluoromethyl)pyridine

To a stirred solution of 2-(trifluoromethyl)pyridin-4-amine (1.0 g, 6.10 mmol, 1.0 eq.) in 5N HCl (10 mL) was added NaNO₂ (0.63 g, 0.91 mmol, 1.5 eq.) drop wise dissolved in water (10 mL) at 0° C. After 10 min KI (2.1 g, 12.8 mmol, 2.1 eq.) was added drop wise dissolved in (10 mL). The reaction mixture was stirred at same temperature for 10 min and 1 h in RT. Following this, reaction was allowed diluted with ethyl acetate (30 mL). The aqueous layer was separated pH of aqueous layer was adjusted ˜11 by adding 6N NaOH, the layers were separated and the organic layer was washed with 0.3M Na₂S₂O₃ (2×20 mL). The combined organic layers were washed with brine (30 mL), dried over anhydrous Na₂SO₄, filtered and concentrated under vacuum to get the solid residue which was purified by was purified by normal phase silica-gel column chromatography to get the title compound as white solid (0.71 g, 71%). LCMS: 274.0 [M+1]⁺; ¹H NMR (400 MHz, DMSO-d₆) δ ppm 8.45 (d, J=4.89 Hz, 1H) 8.31 (s, 1H) 8.18 (d, J=4.89 Hz, 1H)

### Step-2: Synthesis of 4-(4,4,5,5-tetramethyl-1,3,2-dioxaborolan-2-yl)-2-(trifluoromethyl)pyridine

To a stirred solution of 4-iodo-2-(trifluoromethyl)pyridine (1.0 g, 3.66 mmol, 1.0 eq.) and Bis(pinacolato)diboron (1.02 g, 4.06 mmol, 1.1 eq.) in 1,4-dioxane (15 mL) was added was added KOAc (1.22 g, 12.6 mmol, 3.5 eq.). The reaction mixture was purged with N₂ for about 15 min and Pd(dppf)Cl₂-DCM complex (0.29 g, 0.1 mol %) was added. Reaction mixture was re-purged with N₂ and heated at 100° C. for 16 h. Following this, reaction was allowed to cool to RT and filtered through celite pad, the celite pad washed with ethyl acetate and water. The aqueous layer was separated extracted using ethyl acetate (3×10 mL). The combined organic layers were washed with brine (30 mL), dried over anhydrous Na₂SO₄, filtered and concentrated under vacuum to get the solid residue which was purified by normal phase silica gel column chromatography to get the title compound as off white solid (0.71 g, 73%). ¹H NMR (400 MHz, DMSO-d₆) δ ppm 8.83 (d, J=4.38 Hz, 1H) 7.86-7.93 (m, 2H) 1.33 (s, 12H)

### Step-3: Synthesis of (S)-2-methyl-N—((S)-1-(2′-(trifluoromethyl)-3,4′-bipyridin-6-yl)ethyl)propane-2-sulfinamide

To a stirred solution of (S)—N—((S)-1-(5-bromopyridin-2-yl)ethyl)-2-methylpropane-2-sulfinamide (0.5 g, 1.60 mmol, 1.0 eq.) and 4-(4,4,5,5-tetramethyl-1,3,2-dioxaborolan-2-yl)-2-(trifluoromethyl)pyridine (0.53 g, 1.96 mmol, 1.2 eq.) in dimethoxyethane:H₂O:EtOH (7:3:2 mL) was added K₃PO₄ (0.69 g, 3.2 mmol, 2.0 eq.). The reaction mixture was purged with N₂ for about 15 min and Pd(dppf)Cl₂DCM complex (0.130 g, 0.1 mol %) was added. Reaction mixture was re-purged with N₂ and heated at 100° C. for 1 h under microwave irradiation. Following this, reaction was allowed to cool to RT and filtered through celite pad, the celite pad washed with ethyl acetate and water. The aqueous layer was separated extracted using ethyl acetate (3×10 mL). The combined organic layers were washed with brine (20 mL), dried over anhydrous Na₂SO₄, filtered and concentrated under vacuum to get the solid residue which was purified by normal phase silica gel column chromatography to get the title compound as off white solid (0.29 g, 48%). LCMS: 372.1 [M+1]⁺; ¹H NMR (400 MHz, DMSO-d₆) δ ppm 9.05 (d, J=2.19 Hz, 1H) 8.87 (d, J=5.26 Hz, 1H) 8.37 (dd, J=8.33, 2.63 Hz, 1H) 8.29 (s, 1H) 8.11-8.17 (m, 1H) 7.73 (d, J=8.33 Hz, 1H) 5.86 (d, J=7.89 Hz, 1H) 4.50-4.58 (m, 1H) 1.47 (d, J=7.02 Hz, 3H) 1.15 (s, 9H)

### Step-4: Synthesis of (S)-1-(2′-(trifluoromethyl)-3,4′-bipyridin-6-yl)ethanamine hydrochloride

To a stirred solution of (S)-2-methyl-N—((S)-1-(2′-(trifluoromethyl)-3,4′-bipyridin-6-yl)ethyl)propane-2-sulfinamide (0.29 g, 0.78 mmol, 1.0 eq.) in methanol (5 mL) was added 4N HCl in dioxane (1.0 mL, 3.9 mmol, 5.0 eq.) at RT. The resulting mixture was stirred for 30 min. Following this, the reaction mixture was evaporated under reduced pressure to get solid residue. The obtained solid was washed with diethyl ether, dried under vacuum to get desired product as off white solid (0.14 g, 70%). LCMS: 268.0 [M+1]⁺; ¹H NMR (400 MHz, DMSO-d₆) δ ppm 9.16 (d, J=1.75 Hz, 1H) 8.89 (d, J=4.82 Hz, 1H) 8.64-8.74 (m, 3H) 8.46 (dd, J=8.11, 1.97 Hz, 1H) 8.33 (s, 1H) 8.18 (d, J=4.38 Hz, 1H) 7.76 (d, J=8.33 Hz, 1H) 4.57-4.67 (m, 1H) 1.55 (d, J=6.58 Hz, 3H)

### Step-5: Synthesis of (S)-4-isopropyl-3-(4-((S)-1-(2′-(trifluoromethyl)-3,4′-bipyridin-6-yl)ethylamino)-1,3,5-triazin-2-yl)oxazolidin-2-one

In a microwave vial charged with (S)-3-(4-chloro-1,3,5-triazin-2-yl)-4-isopropyloxazolidin-2-one (0.12 g, 0.49 mmol, 1.0 eq.), (S)-1-(2′-(trifluoromethyl)-3,4′-bipyridin-6-yl)ethanamine hydrochloride (0.119 g, 0.54 mmol, 1.1 eq.) and N, N-Diisopropylethylamine (0.17 mL, 0.99 mmol, 2.0 eq.) in DMSO (3 mL). The resulting mixture was heated at 150° C. for 1 h. Following this, the reaction mixture was allowed to cool to RT, evaporated under reduced pressure. The residue diluted with water (10 mL) and extracted using ethyl acetate (3×10 mL). The combined organic layers were washed with brine (30 mL), dried over anhydrous Na₂SO₄, filtered and concentrated under vacuum to get the solid residue which was purified by was purified normal phase silica-gel column chromatography followed by reversed phase column chromatography to get the title compound as white solid (0.046 g, 21%), UPLC-MS (Method 3): Rt 4.33, m/z 474.2 [M+1]⁺; ¹H NMR (400 MHz, DMSO-d₆) δ ppm 9.05 (d, J=7.02 Hz, 1H) 8.86 (d, J=4.82 Hz, 1H) 8.75 (d, J=7.45 Hz, 1H) 8.62 (d, J=8.33 Hz, 1H) 8.41 (s, 1H) 8.24-8.32 (m, 3H) 8.11 (br. s., 1H) 7.49-7.57 (m, 2H) 5.13-5.20 (m, 1H) 4.57 (br. s., 1H) 4.41 (br. s., 1H) 4.17-4.36 (m, 4H) 1.53 (d, J=7.02 Hz, 3H) 0.90 (d, J=7.02 Hz, 1H) 0.80 (d, J=7.45 Hz, 1H) 0.63 (d, J=7.02 Hz, 2H) 0.53 (d, J=7.02 Hz, 2H)

### Example-9: Synthesis of (S)-3-(4-((S)-1-(5-(4-chlorophenyl)pyridin-2-yl)ethylamino)-1,3,5-triazin-2-yl)-4-isopropyloxazolidin-2-one (Compound 1.12)

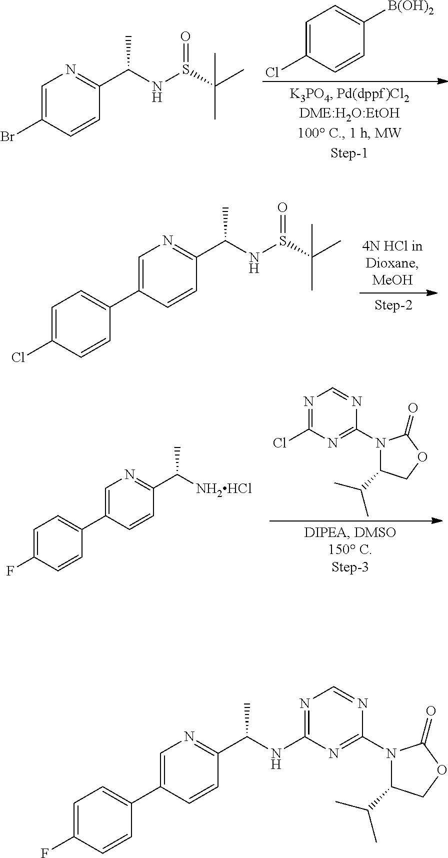

### Step-1: Synthesis of (S)—N—((S)-1-(5-(4-fluorophenyl)pyridin-2-yl)ethyl)-2-methylpropane-2-sulfinamide

To a stirred solution of (S)—N—((S)-1-(5-bromopyridin-2-yl)ethyl)-2-methylpropane-2-sulfinamide (0.5 g, 1.63 mmol, 1.0 eq.) and 4-fluorophenylboronic acid (0.27 g, 1.96 mmol, 1.2 eq.) in dimethoxyethane:H₂O:EtOH (7:3:2 mL) was added K₃PO₄ (0.554 g, 2.612 mmol, 2.0 eq.). The reaction mixture was purged with N₂ for about 15 min and Pd(dppf)Cl₂-DCM complex (0.13 g, 0.1 mol %) was added. Reaction mixture was re-purged with N₂ and heated at 100° C. for 1 h under microwave irradiation. Following this, reaction was allowed to cool to RT and filtered through celite pad, the celite pad washed with ethyl acetate and water. The aqueous layer was separated extracted using ethyl acetate (3×10 mL). The combined organic layers were washed with brine (20 mL), dried over anhydrous Na₂SO₄, filtered and concentrated under vacuum to get the solid residue which was purified by was purified by normal phase silica-gel column chromatography to get the title compound as off white solid (0.2 g, 38%). LCMS: 321.1 [M+1]⁺; ¹H NMR (400 MHz, DMSO-d₆) δ ppm 8.75-8.86 (m, 1H) 8.04-8.12 (m, 1H) 7.78 (dd, J=8.55, 5.48 Hz, 2H) 7.62 (d, J=8.33 Hz, 1H) 7.34 (t, J=8.77 Hz, 2H) 5.78 (d, J=7.89 Hz, 1H) 4.49 (t, J=7.02 Hz, 1H) 1.45 (d, J=7.02 Hz, 3H) 1.15 (s, 9H)

### Step-2: Synthesis of (S)-1-(5-(4-fluorophenyl)pyridin-2-yl)ethanamine hydrochloride

To a stirred solution of (S)—N—((S)-1-(5-(4-fluorophenyl)pyridin-2-yl)ethyl)-2-methylpropane-2-sulfinamide (0.2 g, 0.62 mmol, 1.0 eq.) in methanol (5 mL) was added 4N HCl in dioxane (0.78 mL, 3.12 mmol, 5.0 eq.) at RT. The resulting mixture was stirred for 30 min. Following this, the reaction mixture was evaporated under reduced pressure to get solid residue. The obtained solid was washed with diethyl ether, dried under vacuum to get title compound as off white solid (0.12 g, 88%). LCMS: 217.0 [M+1]⁺; ¹H NMR (400 MHz, DMSO-d₆) δ ppm 8.92 (d, J=1.75 Hz, 1H) 8.60 (br. s., 3H) 8.20 (dd, J=8.11, 2.41 Hz, 1H) 7.78-7.87 (m, 2H) 7.66 (d, J=7.89 Hz, 1H) 7.36 (t, J=8.77 Hz, 2H) 4.53-4.63 (m, 1H) 1.54 (d, J=6.58 Hz, 3H)

### Step-3: Synthesis of (S)-3-(4-((S)-1-(5-(4-fluorophenyl)pyridin-2-yl)ethylamino)-1,3,5-triazin-2-yl)-4-isopropyloxazolidin-2-one

In a microwave vial charged with (S)-3-(4-chloro-1,3,5-triazin-2-yl)-4-isopropyloxazolidin-2-one (0.12 g, 0.49 mmol, 1.0 eq), (S)-1-(5-(4-fluorophenyl)pyridin-2-yl)ethanamine hydrochloride (0.11 g, 0.54 mmol, 1.0 eq.) and N, N-Diisopropylethylamine (0.17 mL, 0.99 mmol, 2.0 eq.), in DMSO (3 mL). The resulting mixture was heated at 150° C. for 1 h. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound as white solid (0.023 g, 11%), UPLC-MS (Method 3): Rt 3.83, m/z 423.3 [M+1]⁺; ¹H NMR (400 MHz, DMSO-d₆) δ ppm 8.79 (d, J=2.19 Hz, 1 H) 8.70 (d, J=7.89 Hz, 1H) 8.55 (d, J=8.33 Hz, 1H) 8.38-8.42 (m, 1H) 7.99-8.04 (m, 2H) 7.71-7.78 (m, 3H) 7.46 (d, J=8.33 Hz, 1H) 7.29-7.41 (m, 5H) 5.25 (s, 1H) 5.09-5.14 (m, 2H) 4.58 (br. s., 1H) 4.41 (d, J=8.33 Hz, 1H) 4.30-4.35 (m, 1H) 4.26 (t, J=8.55 Hz, 2H) 4.16-4.21 (m, 1H) 1.52 (d, J=7.02 Hz, 5H) 1.23 (s, 2H) 0.90 (d, J=7.02 Hz, 2H) 0.79 (d, J=7.02 Hz, 2H) 0.62 (d, J=7.02 Hz, 3H) 0.53 (d, J=7.02 Hz, 3H).

### Example-10: Synthesis of (S)-3-(4-((S)-1-(1-(4-chlorophenyl)-1H-imidazol-4-yl)ethylamino)-1,3,5-triazin-2-yl)-4-isopropyloxazolidin-2-one (Compound 1.13) and (S)-3-(4-((R)-1-(1-(4-chlorophenyl)-1H-imidazol-4-yl)ethylamino)-1,3,5-triazin-2-yl)-4-isopropyloxazolidin-2-one (Compound 1.14)

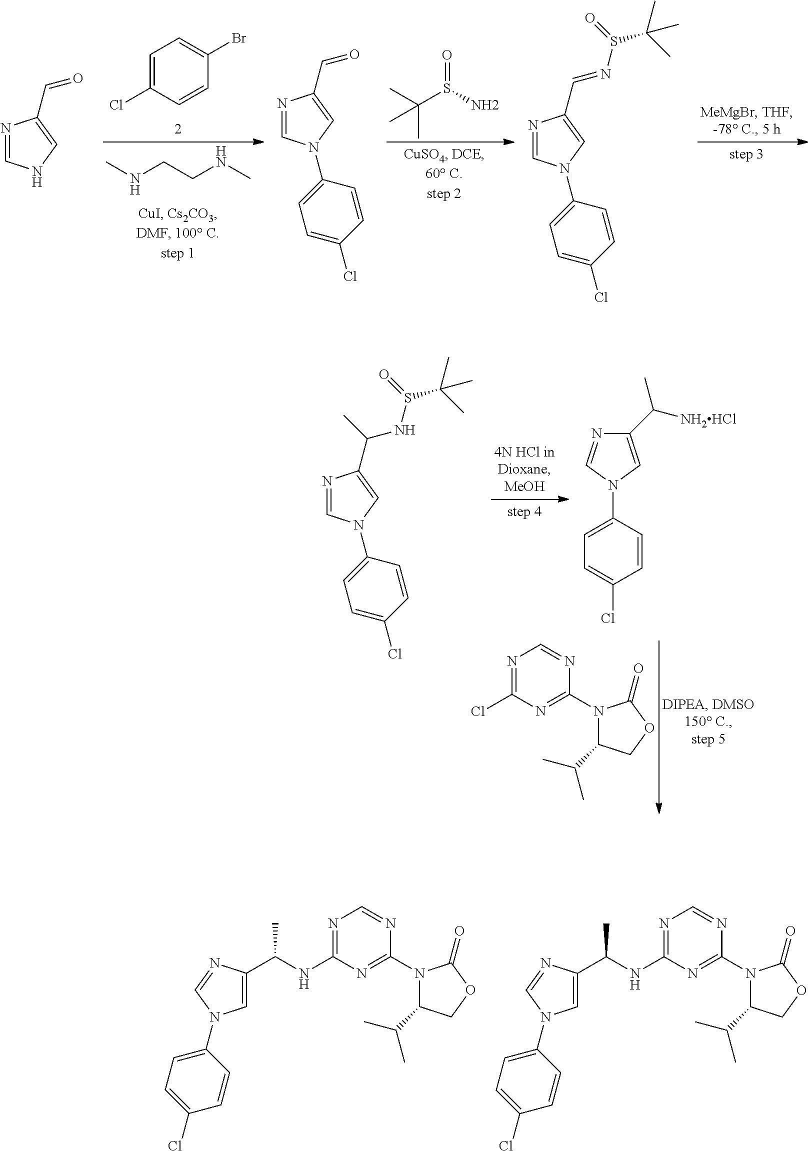

### Step-1: Synthesis of 1-(4-chlorophenyl)-1H-imidazole-4-carbaldehyde

To a stirred solution of 1H-imidazole-4-carbaldehyde (3 g, 31.02 mmol, 1.0 eq.) and 1-bromo-4-chlorobenzene (4.41 g, 37.48 mmol, 1.2 eq.) in DMF (30 mL) was added 1,2-Dimethylethylenediamine (0.52 g, 6.24 mmol, 0.2 eq.) and Cs₂CO₃ (14.48 g, 46.8 mmol, 1.5 eq.). The reaction mixture was purged with N₂ for about 10 min and CuI (0.42 g, 6.2 mmol, 0.1 eq.) was added. Reaction mixture was re-purged with N₂ and heated at 100° C. for 16 h. Following this, reaction was allowed to cool to RT and filtered through celite pad, the celite pad washed with ethyl acetate and water. The aqueous layer was separated extracted using ethyl acetate (3×50 mL). The combined organic layers were washed with brine (150 mL), dried over anhydrous Na₂SO₄, filtered and concentrated under vacuum to get the solid residue which was purified by normal phase silica-gel column chromatography to get the title compound as off white solid (0.86 g, 13%). LCMS: 206.9 [M+1]^{+ 1}H NMR (400 MHz, DMSO-d₆) δ ppm 9.82 (s, 1H) 8.66 (s, 1H) 8.51 (s, 1H) 7.81 (m, J=8.77 Hz, 2H) 7.65 (m, J=8.77 Hz, 2H).

### Step-2: Synthesis of (S,E)-N-((1-(4-chlorophenyl)-1H-imidazol-4-yl)methylene)-2-methylpropane-2-sulfinamide

To a stirred solution of 1-(4-Chlorophenyl)-1H-imidazole-4-carbaldehyde (0.86 g, 4.17 mmol, 1.0 eq.) and Copper(II) sulfate (1.33 g, 8.34 mmol, 2.0 eq.) in dichloroethane (10 mL) was added (S)-2-methylpropane-2-sulfinamide (0.50 g, 4.17 mmol, 1.0 eq.) at RT. The resulting mixture was heated at 60° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound as white solid (0.53 g, 41%). LCMS: 310.0 [M+1]⁺, ¹H NMR (400 MHz, DMSO-d₆) δ ppm 8.57 (br. s., 2H) 8.40 (s, 1H) 7.80 (m, J=8.77 Hz, 2H) 7.64 (m, J=8.33 Hz, 2H) 1.08 (s, 9H)

### Step-3: Synthesis of (S)—N-(1-(1-(4-chlorophenyl)-1H-imidazol-4-yl)ethyl)-2-methylpropane-2-sulfinamide

To a stirred solution of (S,E)-N-((1-(4-chlorophenyl)-1H-imidazol-4-yl)methylene)-2-methylpropane-2-sulfinamide (0.53 g, 1.71 mmol, 1.0 eq.) in THF (10 mL) was added drop wise 3Mmethylmagnesium bromide (0.85 mL, 2.57 mmol, 1.5 eq.) at −78° C. The resulting mixture was stirred for 5 h at same temperature. The reaction was then quenched by careful addition of saturated NH₄Cl (10 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound as semi solid (0.2 g 35.9%). LCMS: 326.0 [M+1]⁺; ¹H NMR (400 MHz, DMSO-d₆) δ ppm 8.22 (d, J=9.65 Hz, 1H) 7.64-7.69 (m, 2H) 7.57-7.63 (m, 2H) 7.54 (d, J=13.15 Hz, 1H) 5.20 (t, J=5.70 Hz, 1H) 4.34 (d, J=6.14 Hz, 1H) 1.43-1.53 (m, 3H) 1.10-1.18 (m, 8H) 1.08 (s, 1H)

### Step-4: Synthesis of 1-(1-(4-chlorophenyl)-1H-imidazol-4-yl)ethanamine hydrochloride

To a stirred solution of (S)—N-(1-(1-(4-chlorophenyl)-1H-imidazol-4-yl)ethyl)-2-methylpropane-2-sulfinamide (0.2 g, 0.614 mmol, 1.0 eq.) in methanol (5 mL) was added 4N HCl in dioxane (0.8 mL, 3.08 mmol, 5.0 eq.) at RT. The resulting mixture was stirred for 30 min. Following this, the reaction mixture was evaporated under reduced pressure to get (0.07 g 98%) crude which was carried forward without any further purification. LCMS: 222.0 [M+1]⁺; ¹H NMR (400 MHz, DMSO-d₆) δ ppm 8.85 (br. s., 1H) 8.58 (br. s., 3H) 8.06 (br. s., 1H) 7.70-7.79 (m, 2H) 7.66 (d, J=7.89 Hz, 2H) 4.45 (br. s., 1H) 1.52-1.65 (m, 3H)

### Step-5: Synthesis of (S)-3-(4-((S)-1-(1-(4-chlorophenyl)-1H-imidazol-4-yl)ethylamino)-1,3,5-triazin-2-yl)-4-isopropyloxazolidin-2-one and (S)-3-(4-((R)-1-(1-(4-chlorophenyl)-1H-imidazol-4-yl)ethylamino)-1,3,5-triazin-2-yl)-4-isopropyloxazolidin-2-one

In a microwave vial charged with 1-(1-(4-chlorophenyl)-1H-imidazol-4-yl)ethanamine hydrochloride (0.07 g, 0.316 mmol, 1.0 eq.), (S)-3-(4-chloro-1,3,5-triazin-2-yl)-4-isopropyloxazolidin-2-one (0.72 g, 0.316 mmol, 1.0 eq.) and N, N-Diisopropylethylamine (0.122 g, 0.95 mmol, 3.0 eq.), in DMSO (2 mL). The resulting mixture was heated at 150° C. for 90 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds. Compound 1.13 (0.002 g, 3%), UPLC-MS (Method 2): Rt 2.52, m/z 428.3 [M+1]⁺; ¹H NMR (400 MHz, DMSO-d₆) δ ppm 8.33-8.40 (m, 1H) 8.20 (br. s., 1H) 7.65 (d, J=8.33 Hz, 1H) 7.68 (d, J=8.77 Hz, 1H) 7.56 (d, J=8.33 Hz, 2H) 5.22 (br. s., 1H) 5.03-5.13 (m, 1H) 4.52 (d, J=9.21 Hz, 1H) 4.21-4.37 (m, 2H) 1.45-1.55 (m, 3H) 0.89 (d, J=7.02 Hz, 1H) 0.79 (d, J=6.58 Hz, 1H) 0.71 (dd, J=19.07, 6.80 Hz, 3H), Compound 1.14 (0.003 g, 3.5%), UPLC-MS (Method 3): Rt 3.64, m/z 428.3 [M+1]⁺; ¹H NMR (400 MHz, DMSO-d₆) δ ppm 8.29-8.40 (m, 1H) 8.21 (br. s., 1H) 7.63-7.71 (m, 1H) 7.54-7.60 (m, 1H) 5.18-5.27 (m, 1H) 5.08-5.18 (m, 1H) 4.54 (d, J=8.33 Hz, 1H) 4.26-4.37 (m, 1H) 1.49 (d, J=7.02 Hz, 2H) 0.84-0.92 (m, 2H) 0.79 (dd, J=6.80, 3.29 Hz, 2H)

### Example-11: Synthesis of (S)-3-(4-((R)-1-(3,4-dichlorophenyl)ethylamino)-1,3,5-triazin-2-yl)-4-isopropyloxazolidin-2-one (Compound 1.15) and (S)-3-(4-((S)-1-(3,4-dichlorophenyl)ethylamino)-1,3,5-triazin-2-yl)-4-isopropyloxazolidin-2-one (Compound 1.16)

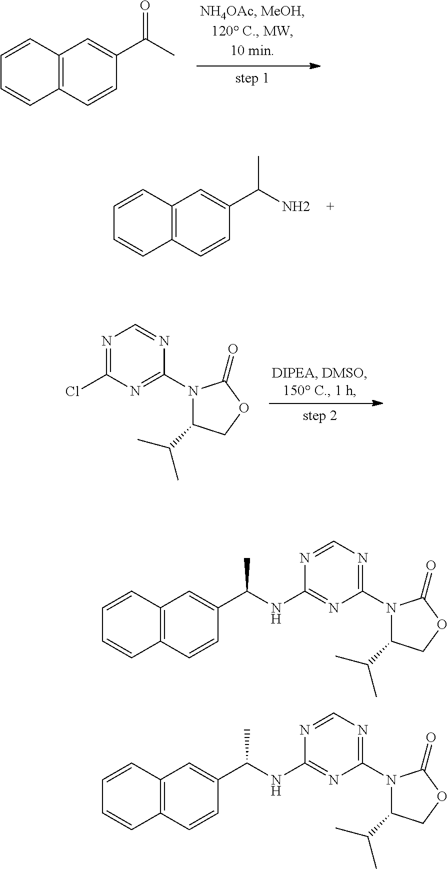

### Step-1: Synthesis of 1-(naphthalen-2-yl)ethanamine

In a microwave vial charged with 1-(naphthalen-2-yl)ethanone (0.5 g, 2.93 mmol, 1.0 eq.), Ammonium acetate (2.26 g, 29.3 mmol, 10.0 eq.) and sodium cyanoborohydride (0.273 g, 4.40 mmol, 1.5 eq.), in MeOH (10 mL). The resulting mixture was heated at 120° C. for 10 min. Following this, the reaction mixture was allowed to cool to RT, basified with 6N NaOH until pH˜10 and extracted with EtOAc (3×15 mL). The combined organic layers were washed with brine (25 mL), dried over Na₂SO₄ and concentrated under reduced pressure to get the title compound as half white solid which was carried forward without any further purification (0.3 g 59%). ¹H NMR (400 MHz, DMSO-d₆) δ ppm 7.76-7.90 (m, 4H) 7.37-7.61 (m, 3H) 4.15 (q, J=6.58 Hz, 1H) 1.31-1.35 (m, 3H).

### Step-2: Synthesis of (S)-3-(4-((R)-1-(3,4-dichlorophenyl)ethylamino)-1,3,5-triazin-2-yl)-4-isopropyloxazolidin-2-one and (S)-3-(4-((S)-1-(3,4-dichlorophenyl)ethylamino)-1,3,5-triazin-2-yl)-4-isopropyloxazolidin-2-one

In a microwave vial charged with 1-(naphthalen-2-yl)ethanamine (0.15 g, 0.877 mmol, 1.0 eq.), (S)-3-(4-chloro-1,3,5-triazin-2-yl)-4-isopropyloxazolidin-2-one (0.212 g, 0.877 mmol, 1.0 eq.) and N, N-Diisopropylethylamine (0.32 g, 2.63 mmol, 3.0 eq.), in DMSO (5 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds; Compound 1.15 (0.024 g 8%), UPLC-MS (Method 4): Rt 2.64, m/z 378.3 [M+1]⁺; ¹H NMR (400 MHz, DMSO-d₆) δ ppm 8.79 (d, J=7.89 Hz, 1H) 8.33-8.41 (m, 1H) 7.80-7.93 (m, 4H) 7.57 (d, J=8.33 Hz, 1H) 7.43-7.52 (m, 2H) 5.29-5.41 (m, 1H) 5.21 (quin, J=7.24 Hz, 1H) 4.51-4.59 (m, 1H) 4.21-4.36 (m, 3H) 1.54 (d, J=7.02 Hz, 3H) 0.83-0.96 (m, 3H) 0.73-0.83 (m, 3H), Compound 1.16 (0.023 g 7.8%, UPLC-MS (Method 4): Rt 2.60, m/z 378.3 [M+1]⁺; ¹H NMR (400 MHz, DMSO-d₆) δ ppm 8.81 (d, J=7.89 Hz, 1H) 8.70 (d, J=8.33 Hz, 1H) 8.35-8.40 (m, 1H) 7.78-7.91 (m, 5H) 7.43-7.61 (m, 4H) 5.19-5.39 (m, 2H) 4.56 (dd, J=8.11, 3.29 Hz, 1H) 4.44-4.52 (m, 1H) 4.17-4.36 (m, 3H) 1.91 (td, J=6.91, 3.73 Hz, 1H) 1.54 (d, J=7.02 Hz, 4H) 0.87 (d, J=7.02 Hz, 2H) 0.77 (d, J=7.02 Hz, 1H) 0.64 (d, J=7.02 Hz, 3H) 0.48 (d, J=6.58 Hz, 3H)

### Example-12: Synthesis of (S)-4-methyl-3-(4-((R)-1-(4-phenoxyphenyl)ethylamino)-1,3,5-triazin-2-yl)oxazolidin-2-one (Compound 1.17)

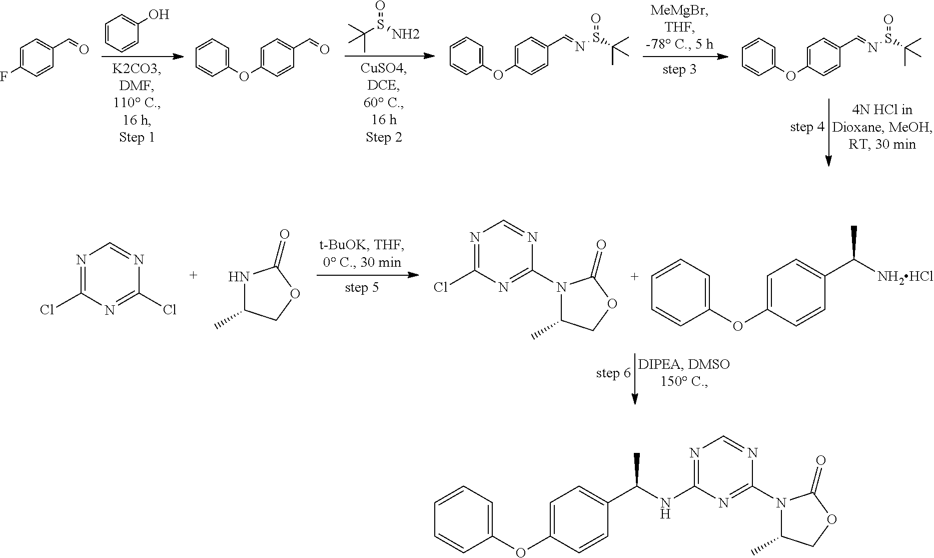

### Step-1: Synthesis of 4-phenoxybenzaldehyde

To a stirred solution of 4-fluorobenzaldehyde (2 g, 8.19 mmol, 21.0 eq.) and phenol (2.63 g, 21.0 mmol, 1.0 eq.) in DMF (20 mL) was added K₂CO₃ (8.6 g, 63.0 mmol, 3 eq.) The resulting mixture heated at 110° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound as white solid (3.2 g, 76%). LCMS: 199.1 [M+1]⁺; ¹H NMR (400 MHz, CHLOROFORM-d) δ ppm 9.93 (s, 1H) 7.85 (d, J=8.77 Hz, 2H) 7.42 (t, J=8.11 Hz, 2H) 7.20-7.28 (m, 2H) 7.03-7.13 (m, 4H).

### Step-2: Synthesis of (S,E)-2-methyl-N-(4-phenoxybenzylidene)propane-2-sulfinamide

To a stirred solution of 4-phenoxybenzaldehyde (3.1 g, 15.1 mmol, 1.0 eq.) and Copper(II) sulfate (7.2 g, 45.3 mmol, 3.0 eq.) in dichloroethane (30 mL) was added (S)-2-methylpropane-2-sulfinamide (1.83 g, 15.1 mmol, 1.0 eq.) at RT. The resulting mixture was heated at 60° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound as semi solid (1.5 g, 33%). LCMS: 302.1 [M+1]⁺; ¹H NMR (400 MHz, DMSO-d₆) δ ppm 8.51 (s, 1H) 7.96 (d, J=8.33 Hz, 2H) 7.46 (t, J=7.89 Hz, 2H) 7.22-7.28 (m, 1H) 7.09 (d, J=8.77 Hz, 2H) 7.13 (d, J=8.33 Hz, 2H) 1.17 (s, 9H)

### Step-3: Synthesis of (S)-2-methyl-N—((R)-1-(4-phenoxyphenyl)ethyl)propane-2-sulfinamide

To a stirred solution of (S,E)-2-methyl-N-(4-phenoxybenzylidene)propane-2-sulfinamide (1.0 g, 3.32 mmol, 1.0 eq.) in THF (10 mL) was added drop wise 3Mmethylmagnesium bromide (1.66 mL, 4.95 mmol, 1.5 eq.) at −78° C. The resulting mixture was stirred for 5 h at same temperature. The reaction was then quenched by careful addition of saturated NH₄Cl (30 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound as white solid (0.8 g 76%). LCMS: 360.2 [M+1]⁺; ¹H NMR (400 MHz, DMSO-d₆) δ ppm 7.31-7.43 (m, 4H) 7.10-7.17 (m, 1H) 6.95 (d, J=8.77 Hz, 2H) 6.99 (d, J=7.45 Hz, 2H) 5.33 (d, J=5.26 Hz, 1H) 4.40 (d, J=6.14 Hz, 1H) 1.45 (d, J=7.02 Hz, 3H) 1.10 (s, 9H)

### Step-4: Synthesis of (R)-1-(4-phenoxyphenyl)ethanamine hydrochloride

To a stirred solution of (S)-2-methyl-N—((R)-1-(4-phenoxyphenyl)ethyl)propane-2-sulfinamide (0.8 g, 2.5 mmol, 1.0 eq.) in methanol (10 mL) was added 4N HCl in dioxane (0.94 mL, 3.78 mmol, 1.5 eq.) at RT. The resulting mixture was stirred for 30 min. Following this, the reaction mixture was evaporated under reduced pressure to get solid. This solid washed with ether and evaporated to give title compound as white color solid (0.52 g 93%). LCMS: 214.1 [M+1]⁺; ¹H NMR (400 MHz, DMSO-d₆) δ ppm 8.47 (br. s., 2H) 7.49-7.58 (m, 2H) 7.37-7.45 (m, 2H) 7.13-7.20 (m, 1H) 6.94-7.09 (m, 4H) 4.37-4.44 (m, 1H) 1.51 (d, J=6.58 Hz, 3H)

### Step-5: Synthesis of (S)-3-(4-chloro-1,3,5-triazin-2-yl)-4-methyloxazolidin-2-one

To a stirred solution of (S)-4-methyloxazolidin-2-one (0.407 g, 4.05 mmol, 1.0 eq.) in THF (10 mL) was added 1 M potassium ter-butoxide solution (0.751 g, 8.05 mmol, 2.0 eq.) at 0° C. The resulting mixture stirred for 15 min at same temperature. To the above solution added 2,4-dichloro-1,3,5-triazine (0.5 g, 3.642 mmol, 0.9 eq.) dissolved in THF (2 mL). The resulting mixture the resulting mixture stirred for another 2 h at same temperature. Following this, the reaction diluted with saturated NH₄Cl (20 mL) and extracted with ethyl acetate (3×15 mL). The combined organic layers were washed with brine (30 mL), dried over anhydrous Na₂SO₄, filtered and concentrated under vacuum to get the solid residue which was purified by was purified normal phase silica-gel column chromatography provided title compound as semi solid (0.2 g. 23%). ¹H NMR (400 MHz, DMSO-d₆) δ ppm 9.02 (s, 1H) 4.68-4.76 (m, 1H) 4.49-4.55 (m, 1H) 4.13 (dd, J=8.33, 3.07 Hz, 1H) 1.40 (d, J=6.58 Hz, 3H).

### Step-6: Synthesis of (S)-4-methyl-3-(4-((R)-1-(4-phenoxyphenyl)ethylamino)-1,3,5-triazin-2-yl)oxazolidin-2-one

In a microwave vial charged with (R)-1-(4-phenoxyphenyl)ethanamine hydrochloride (0.1 g, 0.469 mmol, 1.0 eq.), (S)-3-(4-chloro-1,3,5-triazin-2-yl)-4-methyloxazolidin-2-one (0.11 g, 0.516 mmol, 1.1 eq.) in DMSO (2 mL) was added N, N-Diisopropylethylamine (0.181 g, 1.407 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound as white solid (0.014 g, 9%), UPLC-MS (Method 4): Rt 2.59, m/z 392.3 [M+1]⁺; ¹H NMR (400 MHz, DMSO-d₆) δ ppm 8.55-8.68 (m, 1H) 7.30-7.46 (m, 3H) 7.12 (t, J=7.24 Hz, 1H) 6.90-7.04 (m, 3H) 5.06-5.24 (m, 1H) 4.57-4.68 (m, 1H) 4.36-4.57 (m, 2H) 3.97-4.04 (m, 1H) 1.36-1.49 (m, 4H)

### Example-13: Synthesis of (S)-4-isopropyl-3-(4-((R)-1-(5-phenoxypyridin-2-yl)ethylamino)-1,3,5-triazin-2-yl)oxazolidin-2-one (Compound 1.18) and (S)-4-isopropyl-3-(4-((S)-1-(5-phenoxypyridin-2-yl)ethylamino)-1,3,5-triazin-2-yl)oxazolidin-2-one (Compound 1.19)

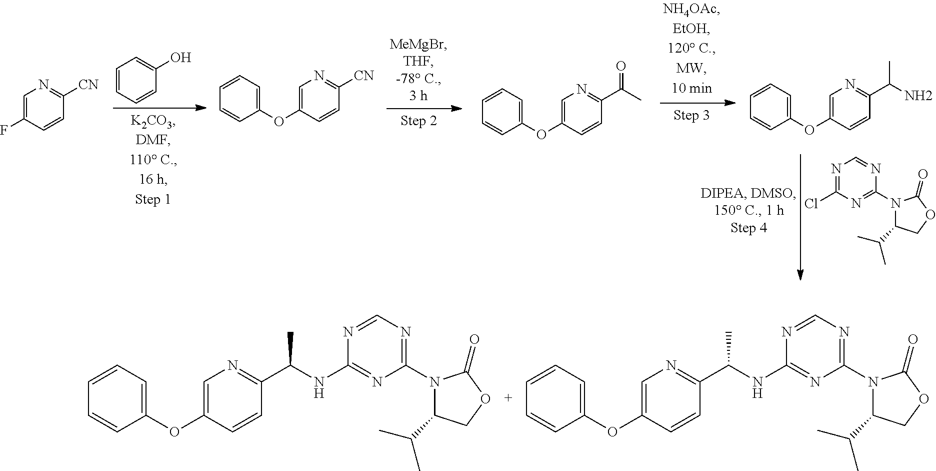

### Step-1: Synthesis of 5-phenoxypicolinonitrile

To a stirred solution of 5-fluoropicolinonitrile (1 g, 8.19 mmol, 1.0 eq.) and phenol (0.92 g, 9.83 mmol, 1.2 eq.) in DMF (10 mL) was added K₂CO₃ (3.35 g, 24.59 mmol, 3 eq.) The resulting mixture heated at 110° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound as white solid (0.7 g, 65%). LCMS: 213.0 [M+1]⁺

### Step-2: Synthesis of 1-(5-phenoxypyridin-2-yl)ethanone

To a stirred solution of 5-phenoxypicolinonitrile (0.7 g, 3.57 mmol, 1.0 eq.) in THF (10 mL) was added drop wise 3 molar methylmagnesium bromide (0.851 g, 7.14 mmol, 2.0 eq.) at −78° C. The resulting mixture was stirred for 3 h at same temperature. The reaction was then quenched by careful addition of saturated NH₄Cl (30 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound as off white solid (0.4 g, 52%). ¹H NMR (400 MHz, CHLOROFORM-d) δ ppm 8.40 (d, J=2.63 Hz, 1H) 8.03 (d, J=8.77 Hz, 1H) 7.39-7.46 (m, 2H) 7.29 (dd, J=8.77, 3.07 Hz, 1H) 7.22-7.25 (m, 1H) 7.09 (dd, J=8.55, 1.10 Hz, 2H) 2.70 (s, 3H)

### Step-3: Synthesis of 1-(5-phenoxypyridin-2-yl)ethanamine

In a microwave vial charged with 1-(5-phenoxypyridin-2-yl)ethanone (0.4 g, 1.877 mmol, 1.0 eq.), Ammonium acetate (1.44 g, 18.77 mmol, 10.0 eq.) and sodium cyanoborohydride (0.08 g, 1.31 mmol, 0.7 eq.), in EtOH (10 mL). The resulting mixture was heated at 120° C. for 10 min. Following this, the reaction mixture was allowed to cool to RT, basified with 6N NaOH until pH˜10 and extracted with EtOAc (3×10 mL). The combined organic layers were washed with brine (15 mL), dried over Na₂SO₄ and concentrated under reduced pressure to get the title compound as semi solid which was carried forward without any further purification (0.25 g, 32%). LCMS: 215.0 [M+1]⁺; ¹H NMR (400 MHz, DMSO-d₆) δ ppm 8.27 (d, J=3.07 Hz, 1H) 7.48-7.52 (m, 1H) 7.37-7.44 (m, 3H) 7.13-7.19 (m, 1H) 7.02 (d, J=7.45 Hz, 2H) 4.01 (q, J=6.58 Hz, 1H) 1.28 (d, J=7.02 Hz, 3H)

### Step-4: Synthesis of (S)-4-isopropyl-3-(4-((R)-1-(5-phenoxypyridin-2-yl)ethylamino)-1,3,5-triazin-2-yl)oxazolidin-2-one and (S)-4-isopropyl-3-(4-((S)-1-(5-phenoxypyridin-2-yl)ethylamino)-1,3,5-triazin-2-yl)oxazolidin-2-one

In a microwave vial charged with 1-(5-phenoxypyridin-2-yl)ethanamine (0.25 g, 1.167 mmol, 1.0 eq.), (S)-3-(4-chloro-1,3,5-triazin-2-yl)-4-isopropyloxazolidin-2-one (0.25 g, 1.05 mmol, 0.9 eq.) in DMSO (2 mL) was added N, N-Diisopropylethylamine (0.448 g, 3.50 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds as white solid; Compound 1.18 (0.012 g, 3%), UPLC-MS (Method 4): Rt 2.44, m/z 421.3 [M+1]⁺; ¹H NMR (400 MHz, DMSO-d₆) δ ppm 8.54-8.63 (m, 1H) 8.50 (d, J=8.33 Hz, 1H) 8.36-8.42 (m, 1H) 8.30 (d, J=2.19 Hz, 1H) 7.36-7.45 (m, 3H) 7.13-7.21 (m, 1H) 6.99-7.07 (m, 2H) 5.04-5.28 (m, 2H) 4.24-4.36 (m, 3H) 2.44 (t, J=7.02 Hz, 1H) 1.48 (d, J=7.02 Hz, 3H) 1.20-1.27 (m, 3H) 0.75-0.91 (m, 6H), Compound 1.19 (0.010 g, 3%), UPLC-MS (Method 4): Rt 2.41, m/z 421.3 [M+1]⁺; ¹H NMR (400 MHz, DMSO-d₆) δ ppm 8.67 (d, J=7.45 Hz, 1H) 8.50 (d, J=7.89 Hz, 1H) 8.36-8.43 (m, 1H) 8.26-8.35 (m, 2H) 7.33-7.48 (m, 6H) 7.14-7.20 (m, 2H) 6.99-7.07 (m, 3H) 5.17-5.28 (m, 1H) 5.10 (dt, J=14.25, 6.91 Hz, 2H) 4.52-4.61 (m, 1H) 4.43-4.49 (m, 1H) 4.19-4.36 (m, 4H) 1.48 (d, J=7.02 Hz, 5H) 1.22-1.27 (m, 6H) 0.77-0.91 (m, 5H) 0.69 (d, J=7.02 Hz, 3H) 0.62 (d, J=7.02 Hz, 3H)

### Example-14: Synthesis of (S)-4-methyl-3-(4-((R)-1-(4-phenoxyphenyl)ethylamino)-1,3,5-triazin-2-yl)oxazolidin-2-one (Compound 1.20)

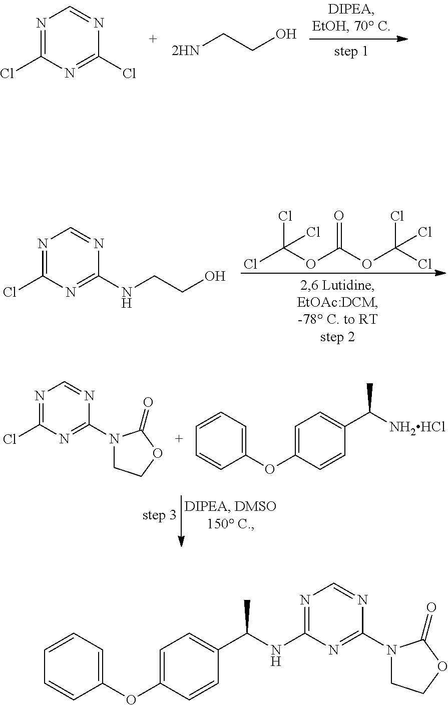

### Step-1: Synthesis of 2-(4-chloro-1,3,5-triazin-2-ylamino)ethanol

To a stirred solution of 2,4-dichloro-1,3,5-triazine (1.0 g, 6.71 mmol, 1.0 eq.) and 2-aminoethanol (0.49 g, 1.02 mmol, 1.2 eq.) in EtOH (10 mL) was added N, N-Diisopropylethylamine (2.59 g, 20.14 mmol, 3.0 eq.) at RT. The resulting mixture was heated at 70° C. for 60 min. Following this, the reaction mixture was allowed to cool to RT and concentrated under vacuum to get crude. The crude diluted with saturated NH₄Cl (10 mL) and extracted with ethyl acetate (3×10 mL). The combined organic layers were washed with brine (30 mL), dried over anhydrous Na₂SO₄, filtered and concentrated under vacuum to get the solid residue which was purified by was purified normal phase silica-gel column chromatography provided title compound as semi solid (0.5 g. 42%). LCMS: 179.9 [M+1]⁺; ¹H NMR (400 MHz, DMSO-d₆) δ ppm 8.60 (d, J=5.70 Hz, 1H) 4.76 (d, J=5.26 Hz, 1H) 3.36-3.40 (m, 2H) 3.32-3.34 (m, 3H).

### Step-2: Synthesis of 3-(4-chloro-1,3,5-triazin-2-yl)oxazolidin-2-one

To a stirred solution of 2-(4-chloro-1,3,5-triazin-2-ylamino)ethanol (0.5 g, 2.87 mmol, 1.0 eq.) and 2,6 Lutidine (1.38 g, 13.21 mmol, 4.6 eq.) in EtOAc:DCM (3:3 mL) was added bis(trichloromethyl) carbonate (0.426 g, 1.43 mmol, 0.5 eq.) at −78° C. The resulting mixture was stirred for 15 min then, removed cooling bath and allowed to warm to RT. The resulting mixture heated at 60° C. for 4 h. Following this, the reaction mixture was allowed to cool to RT. diluted with water (10 mL) and saturated NaHCO₃ solution (10 mL) and extracted with DCM (3×10 mL). The combined organic layers were washed with brine (30 mL), dried over anhydrous Na₂SO₄, filtered and concentrated under vacuum to get the solid residue which was purified by was purified normal phase silica-gel column chromatography provided title compound as semi solid (0.35 g. 60%). ¹H NMR (400 MHz, CHLOROFORM-d) δ ppm 8.89 (s, 1H) 4.54-4.58 (m, 2H) 4.29 (d, J=8.33 Hz, 3H)

### Step-3: Synthesis of (S)-4-methyl-3-(4-((R)-1-(4-phenoxyphenyl)ethylamino)-1,3,5-triazin-2-yl)oxazolidin-2-one

In a microwave vial charged with (R)-1-(4-phenoxyphenyl)ethanamine hydrochloride (0.1 g, 0.469 mmol, 1.0 eq.), 3-(4-chloro-1,3,5-triazin-2-yl)oxazolidin-2-one (0.103 g, 0.516 mmol, 1.1 eq.) in DMSO (2 mL) was added N, N-Diisopropylethylamine (0.181 g, 1.407 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound as off white solid (0.010 g, 6%), UPLC-MS (Method 4): Rt 2.54, m/z 378.3 [M+1]⁺; ¹H NMR (400 MHz, DMSO-d₆) δ ppm 8.61 (dd, J=19.73, 8.33 Hz, 1H) 8.34 (s, 1H) 7.34-7.45 (m, 4H) 7.12 (t, J=7.45 Hz, 1H) 6.90-7.02 (m, 4H) 5.10-5.24 (m, 1H) 4.32-4.40 (m, 2H) 3.92-4.13 (m, 3H) 1.45 (dd, J=6.80, 4.17 Hz, 3H).

### Example-15: Synthesis of (S)-4-isopropyl-3-(4-((S)-1-(4-methyl-2′-(trifluoromethyl)-3,4′-bipyridin-6-yl)ethylamino)-1,3,5-triazin-2-yl)oxazolidin-2-one (Compound 1.21)

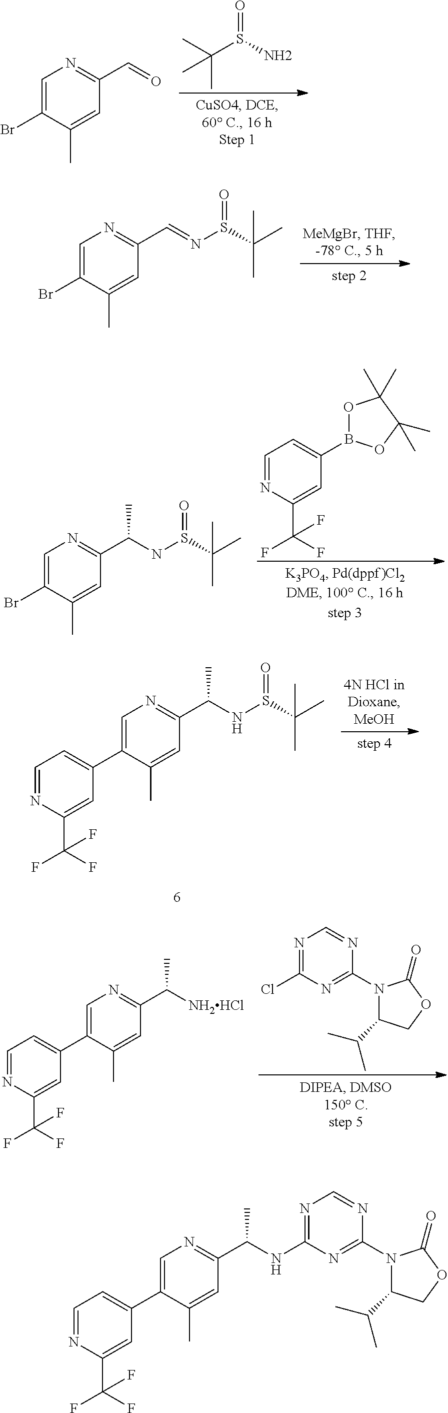

### Step-1: Synthesis of (S,E)-N-((5-bromo-4-methylpyridin-2-yl)methylene)-2-methylpropane-2-sulfinamide

To a stirred solution of 5-bromo-4-methylpicolinaldehyde (0.5 g, 2.51 mmol, 1.0 eq.) and Copper(II) sulfate (1.19 g, 7.53 mmol, 3.0 eq.) in dichloroethane (10 mL) was added (S)-2-methylpropane-2-sulfinamide (0.33 g, 2.76 mmol, 1.1 eq.) at RT. The resulting mixture was heated at 60° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound as brown solid (0.7 g, 92%). LCMS: 303.0 [M+1]⁺; ¹H NMR (400 MHz, DMSO-d₆) δ ppm 8.85 (s, 1H) 8.43 (s, 1H) 8.08 (s, 1H) 1.20 (d, J=2.63 Hz, 12H)

### Step-2: Synthesis of (S)—N—((S)-1-(5-bromo-4-methylpyridin-2-yl)ethyl)-2-methylpropane-2-sulfinamide

To a stirred solution of (S,E)-N-((5-bromo-4-methylpyridin-2-yl)methylene)-2-methylpropane-2-sulfinamide (0.7 g, 2.31 mmol, 1.0 eq.) in THF (10 mL) was added drop wise 3 molar methylmagnesium bromide (0.414 g, 3.47 mmol, 1.5 eq.) at −78° C. The resulting mixture was stirred for 5 h at same temperature. The reaction was then quenched by careful addition of saturated NH₄Cl (10 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound as off white solid (0.5 g 68%). ¹H NMR (400 MHz, DMSO-d₆) δ ppm 8.85 (s, 1H) 8.62 (s, 1H) 8.43 (s, 1H) 8.08 (s, 1H) 1.20 (d, J=2.63 Hz, 12H)

### Step-3: Synthesis of (S)-2-methyl-N—((S)-1-(4-methyl-2′-(trifluoromethyl)-3,4′-bipyridin-6-yl)ethyl)propane-2-sulfinamide

To a stirred solution of (S)—N—((S)-1-(5-bromo-4-methylpyridin-2-yl)ethyl)-2-methylpropane-2-sulfinamide (0.5 g, 1.57 mmol, 1.0 eq.) and 4-(4,4,5,5-tetramethyl-1,3,2-dioxaborolan-2-yl)-2-(trifluoromethyl)pyridine (0.472 g, 1.72 mmol, 1.1 eq.) in Dimethoxyethane (10 mL) was added K₃PO₄ (0.63 g, 3.14 mmol, 2.0 eq.). The reaction mixture was purged with N₂ for about 15 min and Pd(dppf)Cl₂DCM complex (0.134 g, 0.1 mol %) was added. Reaction mixture was re-purged with N₂ and heated at 100° C. for 16 h. Following this, reaction was allowed to cool to RT and filtered through celite pad, the celite pad washed with ethyl acetate and water. The aqueous layer was separated extracted using ethyl acetate (3×10 mL). The combined organic layers were washed with brine (10 mL), dried over anhydrous Na₂SO₄, filtered and concentrated under vacuum to get the solid residue which was purified by was purified by normal phase silica-gel column chromatography to get the title compound as off white solid (0.5 g, 83%). LCMS: 386.1 [M+1]⁺

### Step-4: Synthesis of (S)-1-(4-methyl-2′-(trifluoromethyl)-3,4′-bipyridin-6-yl)ethanamine hydrochloride

To a stirred solution of (S)-2-methyl-N—((S)-1-(4-methyl-2′-(trifluoromethyl)-3,4′-bipyridin-6-yl)ethyl)propane-2-sulfinamide (0.5 g, 1.29 mmol, 1.0 eq.) in methanol (10 mL) was added 4N HCl in dioxane (0.071 g, 1.94 mmol, 1.5 eq.) at RT. The resulting mixture was stirred for 30 min. Following this, the reaction mixture was evaporated under reduced pressure to get solid residue. The obtained solid was washed with diethyl ether, dried under vacuum to get title compound as yellow color solid (0.23 g, 63%) LCMS: 282.2 [M+1]⁺; ¹H NMR (400 MHz, DMSO-d₆) δ ppm 8.90 (d, J=4.82 Hz, 1H) 8.56 (s, 1H) 8.51 (br. s., 2H) 8.02 (s, 1H) 7.85 (d, J=5.26 Hz, 1H) 7.58 (s, 1H) 3.37-3.41 (m, 1H) 2.34 (s, 3H) 1.46-1.59 (m, 3H)

### Step-5: Synthesis of (S)-4-isopropyl-3-(4-((S)-1-(4-methyl-2′-(trifluoromethyl)-3,4′-bipyridin-6-yl)ethylamino)-1,3,5-triazin-2-yl)oxazolidin-2-one

In a microwave vial charged with (S)-1-(4-methyl-2′-(trifluoromethyl)-3,4′-bipyridin-6-yl)ethanamine hydrochloride (0.15 g, 0.53 mmol, 1.0 eq.), (S)-1-(5-(4-(trifluoromethyl)phenyl)pyridin-2-yl)ethanamine (0.142 g, 0.58 mmol, 1.1 eq.) and N, N-Diisopropylethylamine (0.206 g, 1.59 mmol, 3.0 eq.), in DMSO (2 mL). The resulting mixture was heated at 150° C. for 60 min. Following this, the reaction mixture was allowed to cool to RT, diluted with water (10 mL) and extracted extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.021 g, 7%), UPLC-MS (Method 2): Rt 2.81, m/z 488.4 [M+1]⁺; ¹H NMR (400 MHz, DMSO-d₆) δ ppm 8.86 (d, J=4.82 Hz, 2H) 8.70 (d, J=7.45 Hz, 1H) 8.57 (d, J=7.89 Hz, 1H) 8.41 (s, 2H) 8.44 (s, 1H) 8.02 (s, 1H) 7.95 (s, 1H) 7.77-7.85 (m, 2H) 7.36-7.44 (m, 2H) 5.09-5.29 (m, 2H) 4.56-4.61 (m, 1H) 4.45 (d, J=7.89 Hz, 1H) 4.20-4.39 (m, 4H) 2.26-2.31 (m, 4H) 1.91 (br. s., 1H) 1.52 (d, J=7.02 Hz, 5H) 0.90 (d, J=7.02 Hz, 2H) 0.79 (d, J=6.58 Hz, 2H) 0.59-0.72 (m, 6H)

### Example-16: Synthesis of (S)-3-(4-((R)-1-(6-chloro-2-oxo-1,2-dihydroquinolin-3-yl)ethylamino)-1,3,5-triazin-2-yl)-4-isopropyloxazolidin-2-one (Compound 1.22) and (S)-3-(4-((S)-1-(6-chloro-2-oxo-1,2-dihydroquinolin-3-yl)ethylamino)-1,3,5-triazin-2-yl)-4-isopropyloxazolidin-2-one (Compound 1.23)

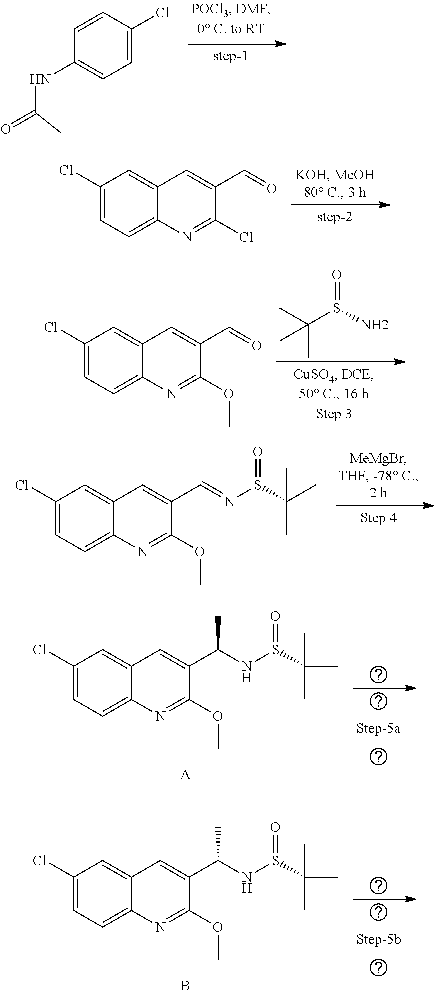

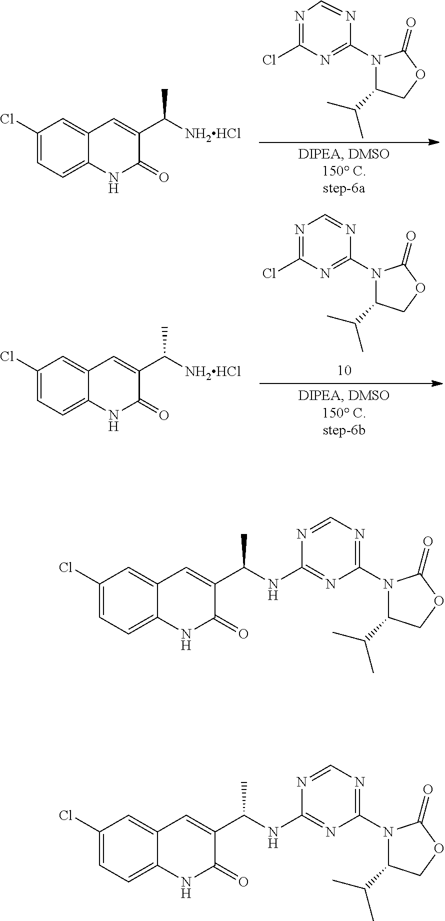

### Step-1: Synthesis of 2,6-dichloroquinoline-3-carbaldehyde

DMF (6.8 mL, 35.37 mmol, 2.5 eq.) was cooled to 0° C. and added POCl₃ (24.6 mL, 247.6 mmol, 7.0 eq.) drop wise over 5 min. To the above solution added N-(4-chlorophenyl)acetamide (6.0 g, 35.37 mmol, 1.0 eq.) dissolved in DMF (10 mL). The resulting mixture heated at 80° C. for 16 h. Following this, reaction mixture diluted with ice cold water (10 mL) and stirred for 1 h. Filtered the solid and under vacuum to get title compound (1.6 g, 20%). ¹H NMR (400 MHz, DMSO-d₆) δ ppm 10.38 (s, 1H) 8.97 (s, 1H) 8.45 (d, J=2.19 Hz, 1H) 8.08 (d, J=9.21 Hz, 1H) 8.00 (dd, J=8.99, 2.41 Hz, 1H)

### Step-2: Synthesis of 6-chloro-2-methoxyquinoline-3-carbaldehyde

To a stirred solution of 2,6-dichloroquinoline-3-carbaldehyde (1.6 g, 7.07 mmol, 1.0 eq.) in MeOH (30 mL) was added KOH (0.513 g, 7.74 mmol, 1.1 eq.) at RT. The resulting mixture was heated to 80° C. and stirred for 3 h at same temperature. Following this, reaction mixture diluted with ice cold water (10 mL) and stirred for 1 h. Filtered the solid and under vacuum to get title compound (1.12 g 74%). ¹H NMR (400 MHz, DMSO-d₆) δ ppm 10.33 (s, 1H) 8.75 (s, 1H) 8.26 (d, J=2.19 Hz, 1H) 7.80-7.88 (m, 2H) 4.12 (s, 3H)

### Step-3: Synthesis of (S,E)-N-((6-chloro-2-methoxyquinolin-3-yl)methylene)-2-methylpropane-2-sulfinamide

To a stirred solution of 6-chloro-2-methoxyquinoline-3-carbaldehyde (3 g, 13.54 mmol, 1.0 eq.) and Copper (II) sulfate (6.46 g, 40.62 mmol, 3.0 eq.) in dichloroethane (30 mL) was added (S)-2-methylpropane-2-sulfinamide (1.97 g, 16.24 mmol, 1.2 eq.) at RT. The resulting mixture was heated at 50° C. for 16 h. Following this, reaction was allowed to cool to room temperature, filtered through celite pad, the celite pad washed with dichloromethane (30 mL). The combined filtrate dried over anhydrous Na₂SO₄ and concentrated under vacuum to get the solid residue which was purified by normal phase silica-gel column chromatography to get the title compound (2.0 g 38%). LCMS: 325.0 [M+1]⁺; ¹H NMR (400 MHz, DMSO-d₆) δ ppm 8.88 (s, 1H) 8.82 (s, 1H) 8.24 (d, J=2.19 Hz, 1H) 7.75-7.86 (m, 2H) 4.10 (s, 3H) 1.22 (s, 9H)

### Step-4: Synthesis of (S)—N—((R)-1-(6-chloro-2-methoxyquinolin-3-yl)ethyl)-2-methylpropane-2-sulfinamide and (S)—N—((S)-1-(6-chloro-2-methoxyquinolin-3-yl)ethyl)-2-methylpropane-2-sulfinamide

To a stirred solution of (S,E)-N-((6-chloro-2-methoxyquinolin-3-yl)methylene)-2-methylpropane-2-sulfinamide (1.6 g, 4.92 mmol, 1.0 eq.) in THF (30 mL) was added drop wise 3M methylmagnesium bromide (4.1 mL, 12.31 mmol, 2.0 eq.) at −78° C. The resulting mixture was stirred for 2 h at same temperature. The reaction was then quenched by careful addition of saturated NH₄Cl (10 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compounds; Compound A (0.54 g, 32%). LCMS: 341.2 [M+1]⁺: Compound B (0.5 g, 31%). LCMS: 341.2 [M+1]⁺

### Step-5a: Synthesis of (R)-3-(1-aminoethyl)-6-chloroquinolin-2(1H)-one hydrochloride

To a stirred solution of (S)—N—((R)-1-(6-chloro-2-methoxyquinolin-3-yl)ethyl)-2-methylpropane-2-sulfinamide (0.5 g, 1.46 mmol, 1.0 eq.) in dioxane (5 mL) was added 4N HCl in dioxane (1.83 mL, 7.33 mmol, 5.0 eq.) at RT. The resulting mixture was heated to reflux for 3 h. Following this, the reaction mixture was evaporated under reduced pressure to get title compound which is used to next step without further purification (0.4 g crude). LCMS: 222.9 [M+1]⁺.

### Step-6a: Synthesis of (S)-3-(4-((R)-1-(6-chloro-2-oxo-1,2-dihydroquinolin-3-yl)ethylamino)-1,3,5-triazin-2-yl)-4-isopropyloxazolidin-2-one

In a microwave vial charged with (R)-3-(1-aminoethyl)-6-chloroquinolin-2(1H)-one hydrochloride (0.13 g, 0.58 mmol, 1.0 eq.), (S)-3-(4-chloro-1,3,5-triazin-2-yl)-4-isopropyloxazolidin-2-one (0.14 g, 0.58 mmol, 1.0 eq.) and N, N-Diisopropylethylamine (0.3 mL, 1.7 mmol, 3.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound as white solid (0.12 g, 48%), UPLC-MS (Method 2): Rt 2.97, m/z 429.3 [M+1]⁺; ¹H NMR (400 MHz, DMSO-d₆) δ ppm 11.99 (br. s., 1H) 8.58 (d, J=7.02 Hz, 1H) 8.38-8.46 (m, 1H) 7.72-7.78 (m, 2H) 7.45-7.52 (m, 1H) 7.30 (dd, J=8.77, 4.82 Hz, 1H) 5.20-5.29 (m, 1H) 5.11-5.20 (m, 1H) 4.53-4.59 (m, 1H) 4.19-4.35 (m, 3H) 2.54 (s, 1H) 1.41 (d, J=7.02 Hz, 3H) 1.21-1.32 (m, 1H) 0.73-0.94 (m, 7H)

### Step-5b: Synthesis of (S)-3-(1-aminoethyl)-6-chloroquinolin-2(1H)-one hydrochloride

To a stirred solution of (S)—N—((S)-1-(6-chloro-2-methoxyquinolin-3-yl)ethyl)-2-methylpropane-2-sulfinamide (0.23 g, 0.64 mmol, 1.0 eq.) in dioxane (5 mL) was added 4N HCl in dioxane (0.84 mL, 3.3 mmol, 5.0 eq.) at RT. The resulting mixture was heated to reflux for 3 h. Following this, the reaction mixture was evaporated under reduced pressure to get title compound which is used to next step without further purification (0.14 g, 94%). LCMS: 222.9 [M+1]⁺.

### Step-6b: Synthesis of (S)-3-(4-((S)-1-(6-chloro-2-oxo-1,2-dihydroquinolin-3-yl)ethylamino)-1,3,5-triazin-2-yl)-4-isopropyloxazolidin-2-one

In a microwave vial charged with (S)-3-(1-aminoethyl)-6-chloroquinolin-2(1H)-one hydrochloride (0.14 g, 0.63 mmol, 1.0 eq.), (S)-3-(4-chloro-1,3,5-triazin-2-yl)-4-isopropyloxazolidin-2-one (0.115 g, 0.63 mmol, 1.0 eq.) and N, N-Diisopropylethylamine (0.4 mL, 1.76 mmol, 3.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.15 g, 55%), UPLC-MS (Method 2): Rt 2.82, m/z 429.3 [M+1]⁺; ¹H NMR (400 MHz, DMSO-d₆) δ ppm 12.00 (br. s., 1H) 8.60 (d, J=6.58 Hz, 1H) 8.36-8.47 (m, 1H) 7.74-7.82 (m, 2H) 7.61 (s, 1H) 7.45-7.52 (m, 1H) 7.31 (d, J=8.77 Hz, 1H) 5.25 (dt, J=13.81, 6.69 Hz, 1H) 5.03 (quin, J=6.69 Hz, 1H) 4.22-4.39 (m, 2H) 4.14 (dd, J=9.21, 2.63 Hz, 1H) 1.73-1.81 (m, 1H) 1.38-1.46 (m, 3H) 1.21-1.31 (m, 1H) 0.77-0.92 (m, 3H) 0.57 (d, J=7.02 Hz, 2H) 0.36 (d, J=6.58 Hz, 2H)

### Example-17: Synthesis of (S)-4-isopropyl-3-(4-((S)-1-(5-(4-methylpiperazin-1-yl)pyridin-2-yl)ethylamino)-1,3,5-triazin-2-yl)oxazolidin-2-one (Compound 1.24) and (S)-4-isopropyl-3-(4-((R)-1-(5-(4-methylpiperazin-1-yl)pyridin-2-yl)ethylamino)-1,3,5-triazin-2-yl)oxazolidin-2-one (Compound 1.25)

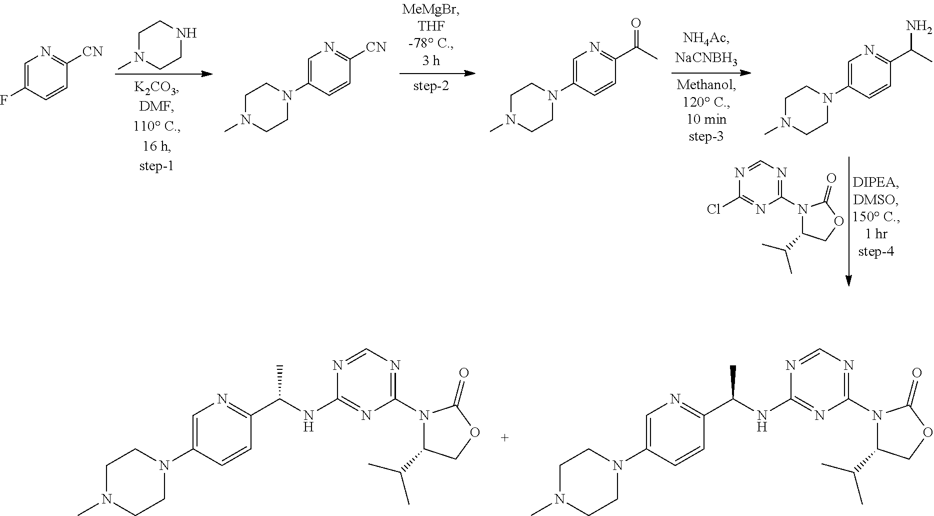

### Step-1: Synthesis of 5-(4-methylpiperazin-1-yl)picolinonitrile

To a stirred solution of 5-fluoropicolinonitrile (1 g, 8.19 mmol, 1.0 eq.) and 1-methylpiperazine (1.62 g, 16.38 mmol, 2.0 eq.) in DMF (15 mL) was added K₂CO₃ (3.35 g, 24.3 mmol, 3 eq.) at RT. The resulting mixture heated at 110° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound as off white solid (0.5 g, 30%). LCMS: 203.2 [M+1]⁺; ¹H NMR (400 MHz, CHLOROFORM-d) δ ppm 8.31 (d, J=2.63 Hz, 1H) 7.51 (d, J=9.21 Hz, 1H) 7.08 (dd, J=8.77, 3.07 Hz, 1H) 3.30-3.47 (m, 4H) 2.50-2.63 (m, 4H) 2.36 (s, 3H)

### Step-2: Synthesis of 1-(5-(4-methylpiperazin-1-yl)pyridin-2-yl)ethanone

To a stirred solution of 5-(4-methylpiperazin-1-yl)picolinonitrile (0.5 g, 2.47 mmol, 1.0 eq.) in THF (10 mL) was added drop wise 3 molar methylmagnesium bromide (0.589 g, 4.94 mmol, 2.0 eq.) at −78° C. The resulting mixture was stirred for 3 h at same temperature. The reaction was then quenched by careful addition of saturated NH₄Cl (30 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound as off white solid (0.4 g, 73.9%). LCMS: 220.0 [M+1]⁺; ¹H NMR (400 MHz, CHLOROFORM-d) δ ppm 8.28 (d, J=3.07 Hz, 1H) 7.96 (d, J=8.77 Hz, 1H) 7.16 (dd, J=8.99, 2.85 Hz, 1H) 3.37-3.43 (m, 4H) 2.65 (s, 3H) 2.55-2.61 (m, 4H) 2.36 (s, 3H)

### Step-3: Synthesis of 1-(5-(4-methylpiperazin-1-yl)pyridin-2-yl)ethanamine

In a microwave vial charged with 1-(5-(4-methylpiperazin-1-yl)pyridin-2-yl)ethanone (0.4 g, 1.877 mmol, 1.0 eq.), Ammonium acetate (1.44 g, 18.77 mmol, 10.0 eq.) and sodium cyanoborohydride (0.08 g, 1.26 mmol, 0.7 eq.), in MeOH (10 mL). The resulting mixture was heated at 120° C. for 10 min. Following this, the reaction mixture was allowed to cool to RT, basified with 6N NaOH until pH˜10 and extracted with EtOAc (3×10 mL). The combined organic layers were washed with brine (15 mL), dried over Na₂SO₄ and concentrated under reduced pressure to get the title compound as semi solid which was carried forward without any further purification (0.21 g, 50%). ¹H NMR (400 MHz, DMSO-d₆) δ ppm 8.18 (d, J=2.63 Hz, 1H) 7.26-7.33 (m, 2H) 3.15 (dd, J=9.87, 5.04 Hz, 4H) 2.42-2.47 (m, 4H) 2.21 (s, 3H) 1.25 (d, J=6.58 Hz, 3H)

### Step-4: Synthesis of (S)-4-isopropyl-3-(4-((S)-1-(5-(4-methylpiperazin-1-yl)pyridin-2-yl)ethylamino)-1,3,5-triazin-2-yl)oxazolidin-2-one and (S)-4-isopropyl-3-(4-((R)-1-(5-(4-methylpiperazin-1-yl)pyridin-2-yl)ethylamino)-1,3,5-triazin-2-yl)oxazolidin-2-one

In a microwave vial charged with 1-(5-(4-methylpiperazin-1-yl)pyridin-2-yl)ethanamine (0.15 g, 0.7 mmol, 1.0 eq.), (S)-3-(4-chloro-1,3,5-triazin-2-yl)-4-isopropyloxazolidin-2-one (0.16 g, 0.7 mmol, 1.0 eq.) in DMSO (2 mL) was added N, N-Diisopropylethylamine (0.270 g, 2.1 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds; Compound 1.24 (0.013 g, 5%), UPLC-MS (Method 5): Rt 3.21, m/z 427.4 [M+1]⁺; ¹H NMR (400 MHz, DMSO-d₆) δ ppm 8.53 (d, J=7.45 Hz, 1H) 8.34-8.39 (m, 2H) 8.19 (d, J=2.63 Hz, 1H) 7.11-7.33 (m, 4H) 5.07-5.16 (m, 1H) 5.00 (quin, J=7.13 Hz, 2H) 4.55 (dd, J=8.11, 3.73 Hz, 1H) 4.42 (dd, J=8.11, 3.29 Hz, 1H) 4.18-4.38 (m, 4H) 3.10-3.17 (m, 6H) 2.40-2.47 (m, 6H) 2.21 (s, 4H) 1.82-1.90 (m, 5H) 1.41-1.47 (m, 4H) 0.89 (d, J=7.02 Hz, 2H) 0.78 (d, J=7.02 Hz, 2H) 0.69 (d, J=7.02 Hz, 3H) 0.59 (d, J=7.02 Hz, 3H), Compound 1.25 (0.012 g, 4%), UPLC-MS (Method 5): Rt 3.31, m/z 427.4 [M+1]⁺; ¹H NMR (400 MHz, DMSO-d₆) δ ppm 8.47 (d, J=7.89 Hz, 1H) 8.32-8.40 (m, 1H) 8.20 (br. s., 1H) 7.15-7.34 (m, 3H) 4.99-5.18 (m, 2H) 4.55 (dd, J=7.89, 3.51 Hz, 1H) 4.24-4.38 (m, 3H) 3.09-3.18 (m, 4H) 2.41-2.47 (m, 5H) 2.21 (s, 3H) 1.90 (s, 2H) 1.43 (d, J=6.58 Hz, 3H) 0.89 (dd, J=7.02, 1.75 Hz, 3H) 0.75-0.82 (m, 3H)

### Example-18: Synthesis of (S)-4-phenyl-3-(4-((S)-1-(1-(4-(trifluoromethyl)phenyl)-1H-imidazol-4-yl)ethylamino)-1,3,5-triazin-2-yl)oxazolidin-2-one (Compound 1.26)

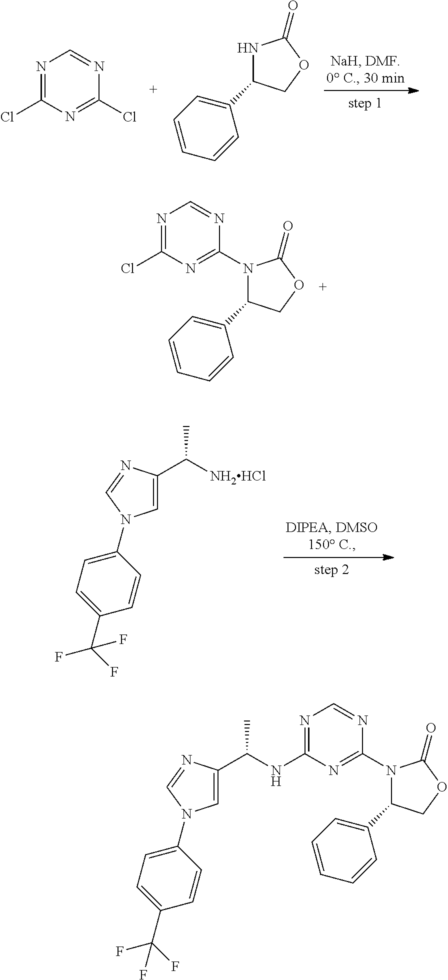

### Step-1: Synthesis of (S)-3-(4-chloro-1,3,5-triazin-2-yl)-4-phenyloxazolidin-2-one

To a stirred solution of Sodium hydride (0.198 g, 5.3 mmol, 1.5 eq.) in DMF (5 mL) was added (S)-4-phenyloxazolidin-2-one (0.59 g, 3.69 mmol, 1.1 eq.) in DMF (3 mL) at 0° C. The resulting solution stirred for 15 min, following this 2,4-dichloro-1,3,5-triazine (0.5 g, 3.35 mmol, 1.0 eq.) in DMF (2 mL) was added. The resulting mixture was stirred for another 30 min at same temperature. Following this, the reaction mixture diluted with saturated NH₄Cl (10 mL) and extracted with ethyl acetate (3×15 mL). The combined organic layers were washed with brine (30 mL), dried over anhydrous Na₂SO₄, filtered and concentrated under vacuum to get the solid residue which was purified by was purified normal phase silica-gel column chromatography provided title compound (0.35 g, 24%). LCMS: 276.9 [M+1]⁺; ¹H NMR (400 MHz, DMSO-d₆) δ ppm 8.95 (s, 1H) 7.30-7.43 (m, 5H) 5.75 (dd, J=8.11, 3.29 Hz, 1H) 4.84 (t, J=8.55 Hz, 1H) 4.28 (dd, J=8.77, 3.51 Hz, 1H)

### Step-2: Synthesis of (S)-4-phenyl-3-(4-((S)-1-(1-(4-(trifluoromethyl)phenyl)-1H-imidazol-4-yl)ethylamino)-1,3,5-triazin-2-yl)oxazolidin-2-one

In a microwave vial charged with (S)-1-(1-(4-(trifluoromethyl)phenyl)-1H-imidazol-4-yl)ethanamine hydrochloride (0.05 g, 0.19 mmol, 1.0 eq.), (S)-3-(4-chloro-1,3,5-triazin-2-yl)-4-phenyloxazolidin-2-one (0.059 g, 0.21 mmol, 1.1 eq.) and N, N-Diisopropylethylamine (0.075 g, 0.58 mmol, 3.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.008 g, 9%), UPLC-MS (Method 2): Rt 2.82, m/z 496.4 [M+1]⁺; ¹H NMR (400 MHz, DMSO-d₆) δ ppm 8.24-8.39 (m, 5H) 7.85-7.93 (m, 6H) 7.76 (s, 1H) 7.62 (s, 1H) 7.28-7.40 (m, 7H) 5.65-5.74 (m, 2H) 4.94-5.03 (m, 2H) 4.77 (t, J=8.33 Hz, 2H) 4.14 (dd, J=8.77, 3.95 Hz, 2H) 1.47 (d, J=7.02 Hz, 1H) 1.20 (d, J=6.58 Hz, 3H)

### Example-19: Synthesis of (S)-4-isopropyl-3-(4-methyl-6-((S)-1-(4-phenoxyphenyl)ethylamino)-1,3,5-triazin-2-yl)oxazolidin-2-one (Compound 1.27)

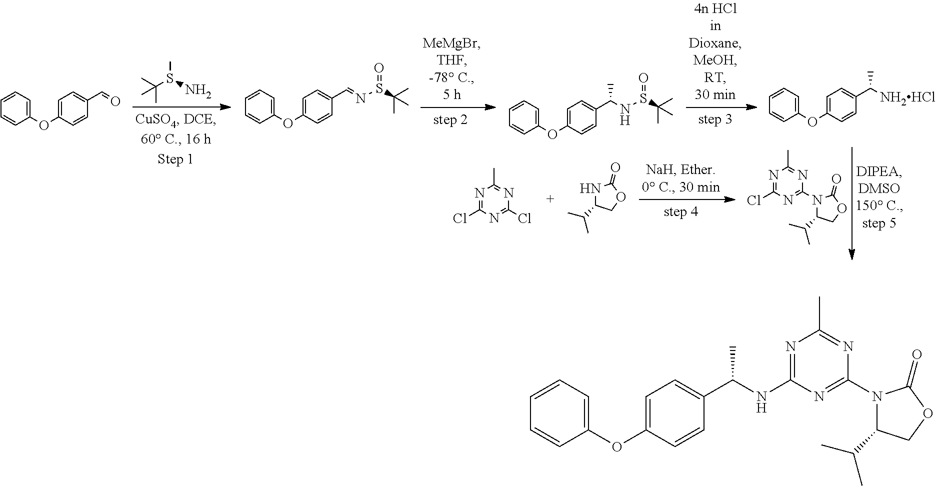

### Step-1: Synthesis of (R,E)-2-methyl-N-(4-phenoxybenzylidene)propane-2-sulfinamide

To a stirred solution of 4-phenoxybenzaldehyde (1.0 g, 5.05 mmol, 1.0 eq.) and Copper(II) sulfate (4.87 g, 15.15 mmol, 3.0 eq.) in dichloroethane (30 mL) was added (R)-2-methylpropane-2-sulfinamide (0.611 g, 5.05 mmol, 1.0 eq.) at RT. The resulting mixture was heated at 60° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound as semi solid (1 g, 65%). LCMS: 302.3 [M+1]⁺; ¹H NMR (400 MHz, DMSO-d₆) δ ppm 8.51 (s, 1H) 7.96 (d, J=8.33 Hz, 2H) 7.46 (t, J=7.89 Hz, 2H) 7.25 (t, J=7.45 Hz, 1H) 7.09 (d, J=8.33 Hz, 2H) 7.13 (d, J=8.33 Hz, 2H) 1.17 (s, 9H)

### Step-2: Synthesis of (R)-2-methyl-N—((S)-1-(4-phenoxyphenyl)ethyl)propane-2-sulfinamide

To a stirred solution of (R,E)-2-methyl-N-(4-phenoxybenzylidene)propane-2-sulfinamide (1.0 g, 3.32 mmol, 1.0 eq.) in THF (10 mL) was added drop wise 3 molar methylmagnesium bromide (1.66 mL, 4.98 mmol, 1.5 eq.) at −78° C. The resulting mixture was stirred for 5 h at same temperature. The reaction was then quenched by careful addition of saturated NH₄Cl (30 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound as white solid (0.85 g, 80%). LCMS: 318.4 [M+1]⁺; ¹H NMR (400 MHz, DMSO-d₆) δ ppm 7.31-7.43 (m, 4H) 7.13 (t, J=7.45 Hz, 1H) 6.89-7.04 (m, 4H) 5.33 (d, J=4.82 Hz, 1H) 4.35-4.43 (m, 1H) 1.45 (d, J=7.02 Hz, 3H) 1.10 (s, 9H)

### Step-3: Synthesis of (S)-1-(4-phenoxyphenyl)ethanamine hydrochloride

To a stirred solution of (R)-2-methyl-N—((S)-1-(4-phenoxyphenyl)ethyl)propane-2-sulfinamide (0.85 g, 2.6 mmol, 1.0 eq.) in methanol (10 mL) was added 4N HCl in dioxane (0.94 mL, 4.03 mmol, 1.5 eq.) at RT. The resulting mixture was stirred for 30 min. Following this, the reaction mixture was evaporated under reduced pressure to get solid. This solid washed with ether and evaporated to give title compound as white color solid (0.52 g, 94%). LCMS: 214.1 [M+1]⁺; ¹H NMR (400 MHz, DMSO-d₆) δ ppm 8.50 (br. s., 2H) 7.54 (d, J=8.77 Hz, 2H) 7.41 (t, J=7.89 Hz, 2H) 7.17 (t, J=7.45 Hz, 1H) 7.01 (d, J=7.89 Hz, 2H) 7.05 (d, J=8.77 Hz, 2H) 4.35-4.44 (m, 1H) 3.50-3.57 (m, 2H) 1.51 (d, J=6.58 Hz, 3H)

### Step-4: Synthesis of (S)-3-(4-chloro-6-methyl-1,3,5-triazin-2-yl)-4-isopropyloxazolidin-2-one

To a stirred solution of Sodium hydride (0.18 g, 4.5 mmol, 1.5 eq.) in Ether (5 mL) was added (S)-4-phenyloxazolidin-2-one (0.435 g, 3.30 mmol, 1.1 eq.) in Ether (3 mL) at 0° C. The resulting solution stirred for 15 min, following this 2,4-dichloro-6-methyl-1,3,5-triazine (0.5 g, 3.06 mmol, 1.0 eq.) in Ether (2 mL) was added. The resulting mixture was stirred for another 30 min at same temperature, concentrated under vacuum to get the solid residue which was purified by was purified normal phase silica-gel column chromatography provided title compound as yellow color solid (0.32 g, 37%). LCMS: 257.1 [M+1]⁺; ¹H NMR (400 MHz, DMSO-d₆) δ ppm 4.58-4.65 (m, 1H) 4.32-4.43 (m, 2H) 2.53 (s, 3H) 2.41 (dt, J=7.02, 3.51 Hz, 1H) 0.91 (d, J=7.45 Hz, 3H) 0.75-0.84 (m, 3H)

### Step-5: Synthesis of (S)-4-isopropyl-3-(4-methyl-6-((S)-1-(4-phenoxyphenyl)ethylamino)-1,3,5-triazin-2-yl)oxazolidin-2-one

In a microwave vial charged with (S)-1-(4-phenoxyphenyl)ethanamine hydrochloride (0.12 g, 0.563 mmol, 1.0 eq.) and (S)-3-(4-chloro-6-methyl-1,3,5-triazin-2-yl)-4-isopropyloxazolidin-2-one (0.144 g, 0.563 mmol, 1.0 eq.) in DMSO (2 mL) was added N,N-Diisopropylethylamine (0.217 g, 1.689 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound as off white solid (0.032 g, 13%), UPLC-MS (Method 4): Rt 2.67, m/z 434.4 [M+1]⁺; ¹H NMR (400 MHz, DMSO-d₆) δ ppm 8.59 (d, J=7.89 Hz, 1H) 7.32-7.42 (m, 5H) 7.09-7.15 (m, 1H) 6.92-7.01 (m, 5H) 5.23-5.29 (m, 1H) 5.04-5.11 (m, 1H) 4.50-4.59 (m, 2H) 4.20-4.35 (m, 3H) 2.25 (s, 3H) 1.40-1.47 (m, 4H) 0.88 (d, J=7.02 Hz, 1H) 0.73-0.79 (m, 4H) 0.64 (d, J=6.58 Hz, 3H)

### Example-20: Synthesis of (S)-4-isopropyl-3-(4-(4-phenoxybenzylamino)-1,3,5-triazin-2-yl)oxazolidin-2-one (Compound 1.28)

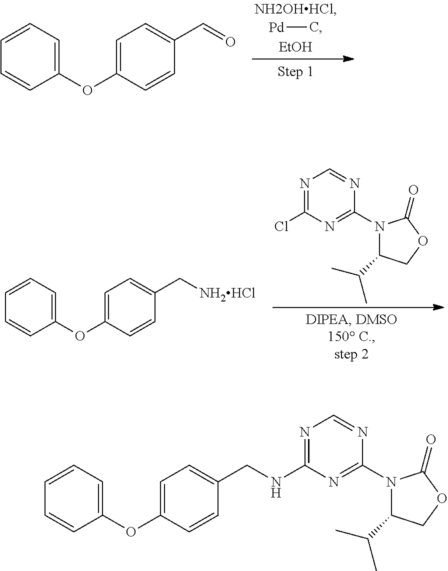

### Step-1: Synthesis of (4-phenoxyphenyl)methanamine hydrochloride

To a stirred solution of 4-phenoxybenzaldehyde (0.5 g, 2.5 mmol, 1.0 eq.) in EtOH:H₂O (9:1 mL) was added Hydroxylammonium chloride (0.17 g, 2.5 mmol, 1.0 eq.) at RT. The resulting mixture stirred for 16 h. Following this, to the above mixture added 10N HCl (0.5 mL) and Pd—C (0.15 g) at RT. The resulting mixture stirred under Hydrogen balloon pressure for 30 min. Following is, reaction was filtered through celite pad, the celite pad washed with ethyl acetate the combined filtered dried over anhydrous Na₂SO₄, filtered and concentrated under vacuum to get the solid residue. The crude used for next step without purification (0.3 g, 60%). ¹H NMR (400 MHz, DMSO-d₆) δ ppm 8.36 (s, 1H) 7.35-7.48 (m, 4H) 7.15 (t, J=7.24 Hz, 1H) 6.94-7.04 (m, 4H) 3.90 (s, 2H)

### Step-2: Synthesis of (S)-4-isopropyl-3-(4-(4-phenoxybenzylamino)-1,3,5-triazin-2-yl)oxazolidin-2-one

In a microwave vial charged with (4-phenoxyphenyl)methanamine hydrochloride (0.15 g, 0.753 mmol, 1.0 eq.), (S)-3-(4-chloro-1,3,5-triazin-2-yl)-4-isopropyloxazolidin-2-one (0.182 g, 0.753 mmol, 1.0 eq.) in DMSO (2 mL) was added N, N-Diisopropylethylamine (0.291 g, 2.26 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound as white solid (0.007 g, 5%), UPLC-MS (Method 7): Rt 3.79, m/z 406.3 [M+1]⁺; ¹H NMR (400 MHz, DMSO-d₆) δ ppm 8.67 (br. s., 1H) 8.59 (br. s., 1H) 8.45 (s, 1H) 8.37 (s, 1H) 7.30-7.42 (m, 5H) 7.12 (t, J=7.45 Hz, 2H) 6.96 (d, J=6.58 Hz, 5H) 4.51 (dd, J=15.35, 6.14 Hz, 4H) 4.40 (dd, J=15.79, 6.14 Hz, 2H) 4.25-4.36 (m, 4H) 0.88 (d, J=7.02 Hz, 2H) 0.77-0.84 (m, 4H) 0.74 (d, J=7.02 Hz, 3H)

### Example-21: Synthesis of (S)-4-ethyl-3-(4-((S)-1-(4-phenoxyphenyl)ethylamino)-1,3,5-triazin-2-yl)oxazolidin-2-one (Compound 1.29)

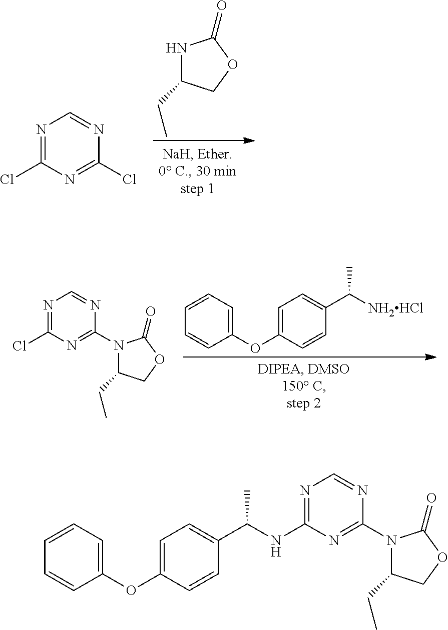

### Step-1: Synthesis of (S)-3-(4-chloro-1,3,5-triazin-2-yl)-4-ethyloxazolidin-2-one

To a stirred solution of Sodium hydride (0.19 g, 4.8 mmol, 1.5 eq.) in Ether (5 mL) was added (S)-4-ethyloxazolidin-2-one (0.212 g, 1.8 mmol, 1.1 eq.) in Ether (3 mL) at 0° C. The resulting solution stirred for 15 min, following this 2,4-dichloro-1,3,5-triazine (0.25 g, 1.6 mmol, 1.0 eq.) in Ether (2 mL) was added. The resulting mixture was stirred for another 30 min at same temperature. On completion of starting material, concentrated the reaction mixture under vacuum to get the solid residue which was purified by normal phase silica-gel column chromatography provided the title compound as color less liquid (0.17 g, 15%). ¹H NMR (400 MHz, DMSO-d₆) δ ppm 9.01 (s, 1H) 4.63 (br. s., 1H) 4.46-4.52 (m, 1H) 4.29-4.38 (m, 1H) 3.89 (dd, J=8.55, 5.92 Hz, 1H) 1.79-1.86 (m, 1H) 1.42-1.48 (m, 1H) 0.86 (dt, J=11.40, 7.45 Hz, 3H)

### Step-2: Synthesis of (S)-4-ethyl-3-(4-((S)-1-(4-phenoxyphenyl)ethylamino)-1,3,5-triazin-2-yl)oxazolidin-2-one

In a microwave vial charged with (S)-1-(4-phenoxyphenyl)ethanamine hydrochloride (0.027 g, 0.13 mmol, 1.0 eq.) and (S)-3-(4-chloro-1,3,5-triazin-2-yl)-4-ethyloxazolidin-2-one (0.03 g, 0.13 mmol, 1.0 eq.) in DMSO (2 mL) was added N, N-Diisopropylethylamine (0.074 g, 0.39 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound (0.005 g, 10%), UPLC-MS (Method 1): Rt 7.29, m/z 406.3 [M+1]⁺; ¹H NMR (400 MHz, DMSO-d₆) δ ppm 8.68 (s, 1H) 8.58 (d, J=8.33 Hz, 1H) 8.34-8.42 (m, 1H) 7.33-7.42 (m, 3H) 7.09-7.15 (m, 1H) 6.92-7.00 (m, 3H) 5.19 (br. s., 1H) 5.06-5.15 (m, 1H) 4.48-4.59 (m, 1H) 4.35-4.42 (m, 1H) 4.12-4.20 (m, 1H) 1.78 (dt, J=14.69, 7.13 Hz, 1H) 1.52 (d, J=10.52 Hz, 1H) 1.40-1.49 (m, 3H) 0.84 (t, J=7.45 Hz, 1H) 0.73 (t, J=7.45 Hz, 2H)

### Example-22: Synthesis of (S)-4-isopropyl-3-(4-((S)-1-(pyridin-2-yl)ethylamino)-1,3,5-triazin-2-yl)oxazolidin-2-one (Compound 1.30)

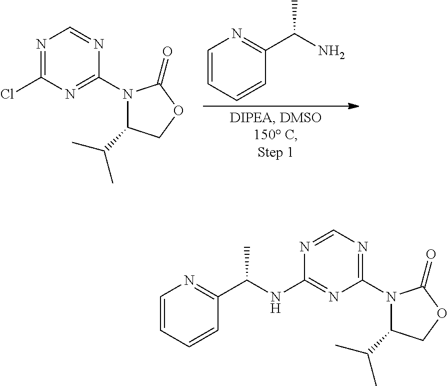

In a microwave vial charged with (S)-1-(pyridin-2-yl)ethanamine (0.1 g, 0.81 mmol, 1.0 eq.), (S)-3-(4-chloro-1,3,5-triazin-2-yl)-4-isopropyloxazolidin-2-one (0.21 g, 0.901 mmol, 1.1 eq.) in DMSO (2 mL) was added N, N-Diisopropylethylamine (0.313 g, 2.45 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography to get the title compound as color less solid (0.008 g, 4%), UPLC-MS (Method 2): Rt 1.44, m/z 329.3 [M+1]⁺; ¹H NMR (400 MHz, DMSO-d₆) δ ppm 8.65 (d, J=7.89 Hz, 1H) 8.49 (d, J=3.51 Hz, 2H) 8.36-8.41 (m, 1H) 7.69-7.78 (m, 2H) 7.19-7.40 (m, 3H) 5.21 (d, J=5.26 Hz, 1H) 5.03-5.12 (m, 1H) 4.53-4.61 (m, 1H) 4.38-4.45 (m, 1H) 4.17-4.37 (m, 4H) 1.86 (s, 3H) 1.72-1.80 (m, 1H) 1.48 (d, J=7.02 Hz, 4H) 0.89 (d, J=7.02 Hz, 1H) 0.79 (d, J=7.02 Hz, 2H) 0.65 (d, J=7.02 Hz, 3H) 0.55 (d, J=6.58 Hz, 3H)

### Example-23: Synthesis of (S)-4-isopropyl-3-(4-methoxy-6-((S)-1-(4-phenoxyphenyl)ethylamino)-1,3,5-triazin-2-yl)oxazolidin-2-one (Compound 1.31)

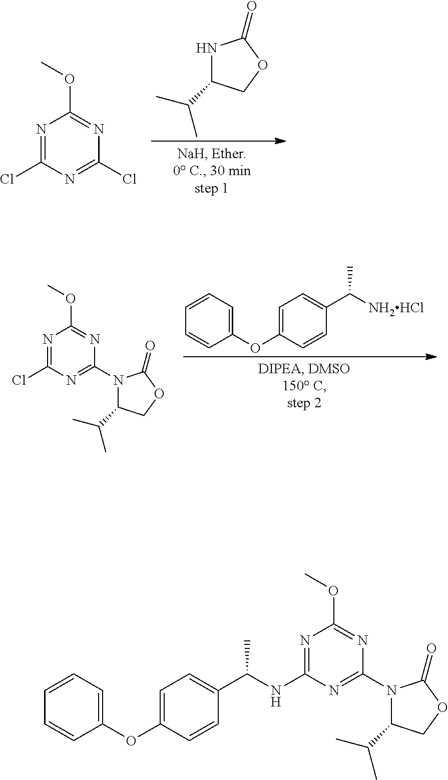

### Step-1: Synthesis of (S)-3-(4-chloro-6-methoxy-1,3,5-triazin-2-yl)-4-isopropyloxazolidin-2-one

To a stirred solution of sodium hydride (0.16 g, 4.5 mmol, 1.5 eq.) in ether (5 mL) was added (S)-4-phenyloxazolidin-2-one (0.39 g, 3.30 mmol, 1.1 eq.) in ether (3 mL) at 0° C. The resulting solution stirred for 15 min, following this 2,4-dichloro-6-methoxy-1,3,5-triazine (0.5 g, 2.7 mmol, 1.0 eq.) in ether (2 mL) was added. The resulting mixture was stirred for another 30 min at same temperature On completion of starting material, concentrated the reaction mixture under vacuum to get the solid residue which was purified by was purified normal phase silica-gel column chromatography provided title compound as color less liquid (0.2 g, 22%). LCMS: 273.1.3 [M+1]⁺

### Step-2: Synthesis of (S)-4-isopropyl-3-(4-methoxy-6-((S)-1-(4-phenoxyphenyl)ethylamino)-1,3,5-triazin-2-yl)oxazolidin-2-one

In a microwave vial charged with (S)-1-(4-phenoxyphenyl)ethanamine hydrochloride (0.1 g, 0.469 mmol, 1.0 eq.) and (S)-3-(4-chloro-6-methoxy-1,3,5-triazin-2-yl)-4-isopropyloxazolidin-2-one (0.14 g, 0.516 mmol, 1.0 eq.) in DMSO (2 mL) was added N, N-Diisopropylethylamine (0.181 g, 1.407 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography to get the title compound as white solid (0.020 g, 10%), UPLC-MS (Method 7): Rt 4.30, m/z 450.4 [M+1]⁺; ¹H NMR (400 MHz, DMSO-d₆) δ ppm 8.54 (dd, J=13.81, 8.11 Hz, 1H) 7.32-7.44 (m, 4H) 7.08-7.17 (m, 1H) 6.90-7.01 (m, 4H) 5.07-5.18 (m, 1H) 4.48-4.57 (m, 1H) 4.20-4.35 (m, 3H) 3.74-3.87 (m, 3H) 2.03-2.10 (m, 1H) 1.39-1.48 (m, 3H) 0.87 (d, J=7.02 Hz, 2H) 0.77 (d, J=7.02 Hz, 3H) 0.66 (d, J=7.02 Hz, 2H)

### Example-24: Synthesis of (S)-3-(4-((S)-1-(6-chloro-2-oxo-1,2-dihydroquinolin-3-yl)ethylamino)-1,3,5-triazin-2-yl)-4-isopropyloxazolidin-2-one (Compound 1.32) and (S)-3-(4-((R)-1-(6-chloro-2-oxo-1,2-dihydroquinolin-3-yl)ethylamino)-1,3,5-triazin-2-yl)-4-isopropyloxazolidin-2-one (Compound 1.33)

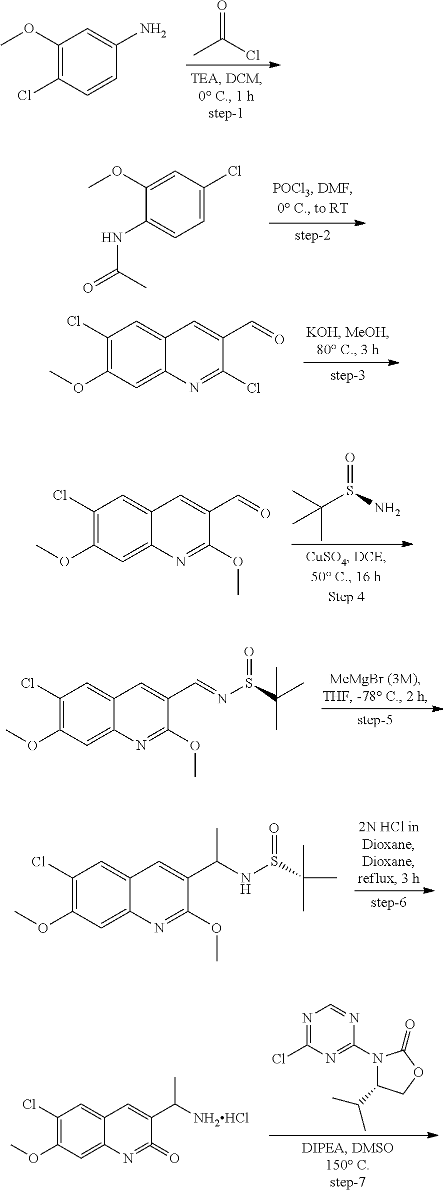

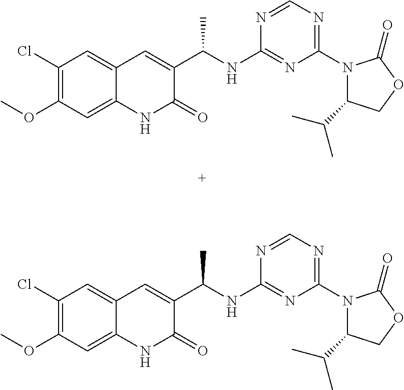 [SMILES: COc1cc2[nH]c(=O)c([C@H](C)Nc3ncnc(N4C(=O)OC[C@@H]4C(C)C)n3)cc2cc1Cl ; COc1cc2[nH]c(=O)c([C@@H](C)Nc3ncnc(N4C(=O)OC[C@@H]4C(C)C)n3)cc2cc1Cl]

### Step-1: Synthesis of N-(4-chlorophenyl)acetamide

To a stirred solution of 4-chloroaniline (2 g, 15.72 mmol, 1.0 eq.) in DCM (20 mL) was added Triethyl amine (6.54 mL, 47.24 mmol, 3.0 eq.). The resulting mixture was cooled to 0° C. and added acetyl chloride (1.68 mL, 23.62 mmol, 1.5 eq.) drop wise. The resulting mixture stirred for 1 h at same temperature. Following this, reaction mixture diluted with water (20 mL). The aqueous layer was separated extracted with DCM (3×30 mL). The combined organic layers were washed with brine (50 mL), dried over anhydrous Na₂SO₄, filtered and concentrated under vacuum to get title compound (2.3 g, 73%). LCMS: 200.0 [M+1]⁺

### Step-2: Synthesis of 2,6-dichloroquinoline-3-carbaldehyde

DMF (9.7 mL, 125.3 mmol, 2.5 eq.) was cooled to 0° C. and added POCl₃ (35 mL, 350.07 mmol, 7.0 eq.) drop wise over 5 min. To the above solution added N-(4-chlorophenyl)acetamide (10.0 g, 50.09 mmol, 1.0 eq.) dissolved in DMF (15 mL). The resulting mixture heated at 80° C. for 16 h. Following this, reaction mixture diluted with ice cold water (10 mL) and stirred for 1 h. Filtered the solid and under vacuum to get title compound (7 g, 54%). ¹H NMR (400 MHz, DMSO-d₆) δ ppm 10.32 (s, 1H) 8.86 (s, 1H) 8.46 (s, 1H) 7.63 (s, 1H) 4.06 (s, 3H)

### Step-3: Synthesis of 6-chloro-2-methoxyquinoline-3-carbaldehyde

To a stirred solution of 2,6-dichloroquinoline-3-carbaldehyde (5 g, 19.53 mmol, 1.0 eq.) in MeOH (50 mL) was added KOH (1.42 g, 21.4 mmol, 1.1 eq.) at RT. The resulting mixture was heated to 80° C. and stirred for 3 h at same temperature. Following this, reaction mixture diluted with ice cold water (10 mL) and stirred for 1 h. Filtered the solid and under vacuum to get title compound (4.0 g 81%). ¹H NMR (400 MHz, DMSO-d₆) δ ppm 10.29 (s, 1H) 8.68 (s, 1H) 8.28 (s, 1H) 7.39 (s, 1H) 4.11 (s, 3H) 4.05 (s, 3H)

### Step-4: Synthesis of (R,E)-N-((6-chloro-2,7-dimethoxyquinolin-3-yl)methylene)-2-methylpropane-2-sulfinamide

To a stirred solution of 6-chloro-2-methoxyquinoline-3-carbaldehyde (1 g, 3.97 mmol, 1.0 eq.) and Copper (II) sulfate (1.26 g, 7.94 mmol, 2.0 eq.) in dichloroethane (10 mL) was added (R)-2-methylpropane-2-sulfinamide (0.48 g, 3.97 mmol, 1.0 eq.) at RT. The resulting mixture was heated at 50° C. for 16 h. Following this, reaction was allowed to cool to room temperature, filtered through celite pad, the celite pad washed with dichloromethane (30 mL). The combined filtrate dried over anhydrous Na₂SO₄ and concentrated under vacuum to get the solid residue which was purified by normal phase silica-gel column chromatography to get the title compound as white solid (1.12 g, 79%). ¹H NMR (400 MHz, CHLOROFORM-d) δ ppm 8.99 (s, 1H) 8.54 (s, 1H) 7.82 (s, 1H) 7.27 (br. s., 1H) 4.13 (s, 3H) 4.06 (s, 3H) 1.29 (s, 9H).

### Step-5: Synthesis of (S)—N-(1-(6-chloro-2,7-dimethoxyquinolin-3-yl)ethyl)-2-methylpropane-2-sulfinamide

To a stirred solution of (R,E)-N-((6-chloro-2,7-dimethoxyquinolin-3-yl)methylene)-2-methylpropane-2-sulfinamide (1.0 g, 2.8 mmol, 1.0 eq.) in THF (20 mL) was added drop wise 3M methylmagnesium bromide (2.3 mL, 6.7 mmol, 2.5 eq.) at −78° C. The resulting mixture was stirred for 2 h at same temperature. The reaction was then quenched by careful addition of saturated NH₄Cl (10 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound as semi solid (0.76 g 67%). ¹H NMR (400 MHz, DMSO-d₆) δ ppm 8.09-8.16 (m, 1H) 7.99-8.03 (m, 1H) 7.29-7.35 (m, 1H) 5.39 (d, J=6.14 Hz, 1H) 4.64-4.72 (m, 1H) 3.96-4.04 (m, 6H) 1.51 (d, J=7.02 Hz, 3H) 1.42 (d, J=7.02 Hz, 1H) 1.06-1.15 (m, 9H)

### Step-6: Synthesis of 3-(1-aminoethyl)-6-chloro-7-methoxyquinolin-2(1H)-one hydrochloride

To a stirred solution of (S)—N-(1-(6-chloro-2,7-dimethoxyquinolin-3-yl)ethyl)-2-methylpropane-2-sulfinamide (0.5 g, 1.34 mmol, 1.0 eq.) in dioxane (5 mL) was added 4N HCl in dioxane (1.6 mL, 6.7 mmol, 5.0 eq.) at RT. The resulting mixture was heated to reflux for 3 h. Following this, the reaction mixture was evaporated under reduced pressure to get title compound which is used to next step without further purification (0.42 g crude). LCMS: 252.9 [M+1]⁺

### Step-7: Synthesis of (S)-3-(4-((S)-1-(6-chloro-2-oxo-1,2-dihydroquinolin-3-yl)ethylamino)-1,3,5-triazin-2-yl)-4-isopropyloxazolidin-2-one and (S)-3-(4-((R)-1-(6-chloro-2-oxo-1,2-dihydroquinolin-3-yl)ethylamino)-1,3,5-triazin-2-yl)-4-isopropyloxazolidin-2-one

In a microwave vial charged with 3-(1-aminoethyl)-6-chloro-7-methoxyquinolin-2(1H)-one hydrochloride (0.35 g, 1.38 mmol, 1.0 eq.), (S)-3-(4-chloro-1,3,5-triazin-2-yl)-4-isopropyloxazolidin-2-one (0.33 g, 1.38 mmol, 1.0 eq.) and N, N-Diisopropylethylamine (0.7 mL, 4.15 mmol, 3.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get title compounds; Compound 1.32 (0.02 g 3%), UPLC-MS (Method 2): Rt 2.81, m/z 459.3 [M+1]⁺; ¹H NMR (400 MHz, DMSO-d₆) δ ppm 11.84 (br. s., 1H) 8.55 (d, J=6.58 Hz, 1H) 8.37-8.42 (m, 2H) 7.76-7.79 (m, 1H) 7.69 (s, 1H) 7.54 (s, 1H) 6.95 (s, 1H) 5.24 (d, J=6.58 Hz, 1H) 4.99-5.05 (m, 1H) 4.57 (br. s., 1H) 4.23-4.41 (m, 4H) 4.15 (dd, J=8.99, 2.85 Hz, 1H) 3.87 (s, 4H) 1.84 (br. s., 2H) 1.37-1.44 (m, 4H) 0.89 (d, J=7.45 Hz, 2H) 0.79 (d, J=6.58 Hz, 2H) 0.60 (d, J=7.02 Hz, 3H) 0.40 (d, J=7.02 Hz, 3H) and Compound 1.33 (0.015 g, 4%), UPLC-MS (Method 6): Rt 4.46, m/z 459.3 [M+1]⁺; ¹H NMR (400 MHz, DMSO-d₆) δ ppm 8.51 (d, J=7.89 Hz, 1H) 8.35-8.41 (m, 2H) 7.76 (d, J=7.02 Hz, 1H) 7.68 (s, 1H) 6.92-6.98 (m, 2H) 5.11-5.16 (m, 1H) 4.20-4.36 (m, 4H) 3.86-3.90 (m, 4H) 1.40 (d, J=6.58 Hz, 3H) 1.35 (d, J=6.58 Hz, 1H) 1.24 (s, 1H) 0.86-0.92 (m, 3H) 0.78 (dd, J=17.32, 6.80 Hz, 4H)

### Example-25: Synthesis of (S)-4-isopropyl-3-(4-((R)-1-(4-phenoxyphenyl)propylamino)-1,3,5-triazin-2-yl)oxazolidin-2-one (Compound No. 1.34) and (S)-4-isopropyl-3-(4-((S)-1-(4-phenoxyphenyl)propylamino)-1,3,5-triazin-2-yl)oxazolidin-2-one (Compound No. 1.35)

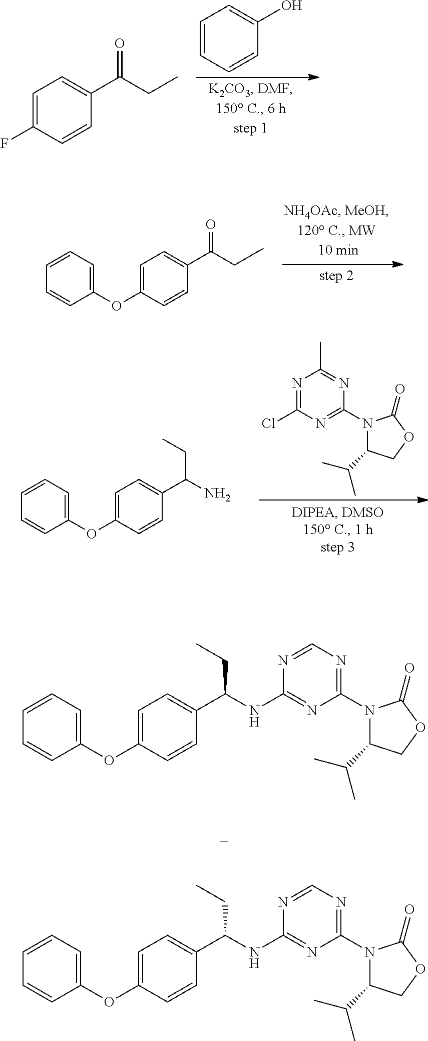

### Step-1: Synthesis of 1-(4-phenoxyphenyl)propan-1-one

To a stirred solution of 1-(4-fluorophenyl)ethanone (2 g, 13.14 mmol, 1.0 eq.) and phenol (1.27 mL, 14.45 mmol, 1.1 eq.) in DMF (20 mL) was added K₂CO₃ (1.18 g, 13.14 mmol, 1 eq.) and heated at 150° C. for 6 h. Following this, reaction was allowed to cool to RT and filtered through celite pad, the celite pad washed with ethyl acetate and water. The aqueous layer was separated extracted using ethyl acetate (3×20 mL). The combined organic layers were washed with brine (30 mL), dried over anhydrous Na₂SO₄, filtered and concentrated under vacuum to get the solid residue which was carried forward without any further purification (1.8 g, 60%). LCMS: 227.3 [M+1]⁺; ¹H NMR (400 MHz, CHLOROFORM-d) δ ppm 7.92-7.98 (m, 2H) 7.35-7.43 (m, 2H) 7.20 (t, J=7.45 Hz, 1H) 7.04-7.10 (m, 2H) 6.97-7.02 (m, 2H) 2.97 (q, J=7.02 Hz, 2H) 1.20-1.26 (m, 4H).

### Step-2: Synthesis of 1-(4-phenoxyphenyl)propan-1-amine

In a microwave vial charged with 1-(4-phenoxyphenyl)propan-1-one (0.5 g, 2.21 mmol, 1.0 eq.), Ammonium acetate (1.70 g, 22.10 mmol, 10.0 eq.) and sodium cyanoborohydride (0.16 g, 2.65 mmol, 1.2 eq.), in MeOH (10 mL). The resulting mixture was heated at 120° C. for 10 min. Following this, the reaction mixture was allowed to cool to RT, basified with 6N NaOH until pH˜10 and extracted with EtOAc (3×15 mL). The combined organic layers were washed with brine (25 mL), dried over Na₂SO₄ and concentrated under reduced pressure to get the title compound which was carried forward without any further purification (0.35 g 69%). ¹H NMR (400 MHz, CHLOROFORM-d) δ ppm 7.29-7.37 (m, 4H) 7.26 (s, 1H) 7.10-7.15 (m, 1H) 6.97-7.02 (m, 4H) 5.41 (br. s., 3H) 4.05 (dd, J=9.21, 5.70 Hz, 1H) 1.87-2.07 (m, 3H) 0.86 (t, J=7.24 Hz, 3H)

### Step-3: Synthesis of (S)-4-isopropyl-3-(4-((R)-1-(4-phenoxyphenyl)propylamino)-1,3,5-triazin-2-yl)oxazolidin-2-one and (S)-4-isopropyl-3-(4-((S)-1-(4-phenoxyphenyl)propylamino)-1,3,5-triazin-2-yl)oxazolidin-2-one

In a microwave vial charged with 1-(4-phenoxyphenyl)propan-1-amine (0.3 g, 1.40 mmol, 1.0 eq.), (S)-3-(4-chloro-1,3,5-triazin-2-yl)-4-isopropyloxazolidin-2-one (0.34 g, 1.40 mmol, 1.0 eq.) and N, N-Diisopropylethylamine (0.75 mL, 4.20 mmol, 3.0 eq.), in DMSO (5 mL). The resulting mixture was heated at 150° C. for 60 min. Following this, the reaction mixture was allowed to cool to RT concentrated under vacuum diluted with water (10 mL) and extracted using ethyl acetate (3×10 mL). The combined organic layers were washed with brine (10 mL), dried over anhydrous Na₂SO₄, filtered and evaporated under reduced pressure to get solid residue. The obtained solid was purified by normal phase silica-gel column chromatography to get chromatography to get title compounds. Further compounds were separated by chiral chromatography to obtain the title compounds; Compound 1.34 (0.010 g, 2%) UPLC-MS (Method 4): Rt 2.80, m/z 434.4 [M+1]⁺; ¹H NMR (400 MHz, DMSO-d₆) δ ppm 8.75 (d, J=8.77 Hz, 1H) 8.36 (s, 1H) 7.33-7.42 (m, 3H) 7.12 (t, J=7.45 Hz, 1H) 6.97 (t, J=8.33 Hz, 3H) 4.90-4.98 (m, 1H) 4.76-4.85 (m, 1H) 4.51-4.59 (m, 1H) 4.42 (dd, J=7.02, 3.95 Hz, 1H) 4.26-4.36 (m, 2H) 1.70-1.92 (m, 2H) 0.77-0.96 (m, 7H) and Compound 1.35 (0.010 g, 2%), UPLC-MS (Method 4): Rt 2.76 m/z 434.4 [M+1]⁺; ¹H NMR (400 MHz, DMSO-d₆) δ ppm 8.82 (d, J=7.89 Hz, 1H) 8.35-8.44 (m, 1H) 7.32-7.44 (m, 3H) 7.08-7.17 (m, 1H) 6.91-7.01 (m, 3H) 4.87-4.98 (m, 1H) 4.77-4.87 (m, 1H) 4.56 (dd, J=7.67, 3.73 Hz, 1H) 4.25-4.36 (m, 2H) 2.05 (td, J=6.69, 3.73 Hz, 1H) 1.69-1.91 (m, 2H) 0.75-0.93 (m, 5H) 0.66 (d, J=7.02 Hz, 2H)

### Example-26: Synthesis of (S)-4-isopropyl-3-(4-((R)-1-phenylpropylamino)-1,3,5-triazin-2-yl)oxazolidin-2-one (Compound No. 1.36) and (S)-4-isopropyl-3-(4-((S)-1-phenylethylamino)-1,3,5-triazin-2-yl)oxazolidin-2-one (Compound No. 1.37)

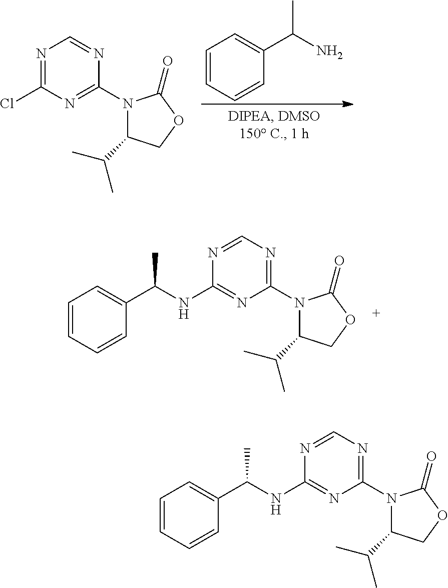

In a microwave vial charged with 1-phenylethanamine (0.079 g, 0.65 mmol, 1.0 eq.), (S)-3-(4-chloro-1,3,5-triazin-2-yl)-4-isopropyloxazolidin-2-one (0.16 g, 0.65 mmol, 1.0 eq.) and N, N-Diisopropylethylamine (0.34 mL, 4.20 mmol, 3.0 eq.), in DMSO (2 mL). The resulting mixture was heated at 150° C. for 60 min. Following this, the reaction mixture was allowed to cool to RT concentrated under vacuum diluted with water (10 mL) and extracted using ethyl acetate (3×10 mL). The combined organic layers were washed with brine (10 mL), dried over anhydrous Na₂SO₄, filtered and evaporated under reduced pressure to get solid residue. The obtained solid was purified by normal phase silica-gel column chromatography to get chromatography to get title compounds. Further compounds were separated by chiral chromatography to obtain title compounds. Compound 1.36 (0.004 g, 2%), UPLC-MS (Method 2): Rt 3.03, m/z 328.4 [M+1]⁺; ¹H NMR (400 MHz, DMSO-d₆) δ ppm 8.67 (d, J=8.77 Hz, 1H) 8.57 (d, J=8.33 Hz, 1H) 8.33-8.40 (m, 1H) 7.79 (br. s., 1H) 7.18-7.40 (m, 4H) 5.18 (br. s., 1H) 4.95-5.07 (m, 1H) 4.26-4.36 (m, 2H) 3.98 (dd, J=8.77, 6.14 Hz, 1H) 3.48-3.56 (m, 1H) 1.59 (dd, J=13.15, 6.58 Hz, 1H) 1.45 (d, J=7.02 Hz, 2H) 1.33-1.38 (m, 1H) 0.75-0.93 (m, 7H); Compound 1.37 (0.005 g, 2%), UPLC-MS (Method 2): Rt 2.99, m/z 328.4 [M+1]⁺; ¹H NMR (400 MHz, DMSO-d₆) δ ppm 8.70 (d, J=7.89 Hz, 1H) 8.57 (d, J=8.33 Hz, 1H) 8.33-8.40 (m, 1H) 7.24-7.42 (m, 4H) 7.16-7.24 (m, 1H) 5.13-5.22 (m, 1H) 5.04-5.13 (m, 1H) 4.47-4.59 (m, 1H) 4.20-4.36 (m, 2H) 1.92-2.06 (m, 1H) 1.39-1.50 (m, 3H) 1.21-1.33 (m, 2H) 0.68-0.94 (m, 5H) 0.61 (d, J=7.02 Hz, 2H).

### Example-27: Synthesis of (S)-3-(4-((S)-1-(6-chloro-2-oxo-1,2-dihydroquinolin-3-yl)ethylamino)-6-methyl-1,3,5-triazin-2-yl)-4-ethyloxazolidin-2-one (Compound 1.38) and (S)-3-(4-((R)-1-(6-chloro-2-oxo-1,2-dihydroquinolin-3-yl)ethylamino)-6-methyl-1,3,5-triazin-2-yl)-4-ethyloxazolidin-2-one (Compound 1.39)

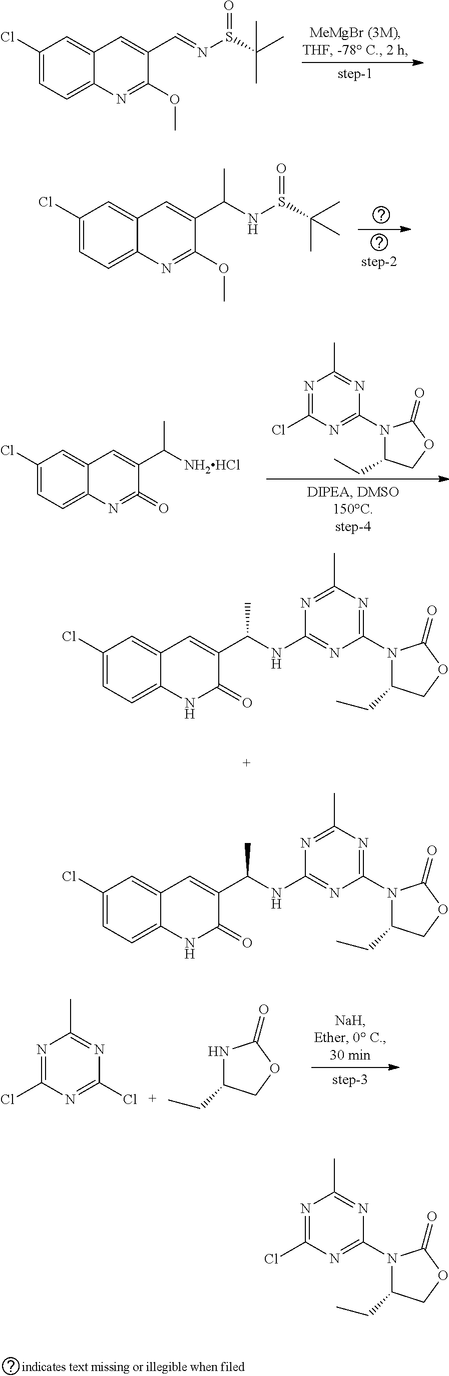

### Step-1: Synthesis of (S)—N-(1-(6-chloro-2-methoxyquinolin-3-yl)ethyl)-2-methylpropane-2-sulfinamide

To a stirred solution of (S,E)-N-((6-chloro-2-methoxyquinolin-3-yl)methylene)-2-methylpropane-2-sulfinamide (1.6 g, 4.92 mmol, 1.0 eq.) in THF (30 mL) was added drop wise 3 molar methylmagnesium bromide (4.1 mL, 12.31 mmol, 2.0 eq.) at −78° C. The resulting mixture was stirred for 2 h at same temperature. The reaction was then quenched by careful addition of saturated NH₄Cl (10 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.8 g 49%). LCMS: 341.1 [M+1]⁺

### Step-2: Synthesis of 3-(1-aminoethyl)-6-chloroquinolin-2(1H)-one hydrochloride

To a stirred solution of (S)—N-(1-(6-chloro-2-methoxyquinolin-3-yl)ethyl)-2-methylpropane-2-sulfinamide (0.3 g, 0.613 mmol, 1.0 eq.) in MeOH (5 mL) was added 4N HCl in dioxane (0.23 mL, 0.92 mmol, 1.5 eq.) at RT. The resulting mixture stirred for 3 h. Following this, the reaction mixture was evaporated under reduced pressure to get title compound which is used to next step without further purification (0.252 g). LCMS: 223.1 [M+1]⁺; ¹H NMR (400 MHz, DMSO-d₆) δ ppm 12.33 (s, 1H) 8.25 (br. s., 2H) 8.06 (s, 1H) 7.85 (d, J=2.19 Hz, 1H) 7.61 (dd, J=8.55, 2.41 Hz, 1H) 7.39 (d, J=8.77 Hz, 1H) 4.43 (d, J=5.70 Hz, 1H) 1.53 (d, J=6.58 Hz, 3H)

### Step-3: Synthesis of (S)-3-(4-chloro-6-methyl-1,3,5-triazin-2-yl)-4-ethyloxazolidin-2-one

To a stirred solution of Sodium hydride (0.072 g, 1.9 mmol, 1.5 eq.) in Ether (5 mL) was added (S)-4-ethyloxazolidin-2-one (0.15 g, 1.30 mmol, 1.1 eq.) in Ether (3 mL) at 0° C. The resulting solution stirred for 15 min, following this 2,4-dichloro-6-methyl-1,3,5-triazine (0.2 g, 1.2 mmol, 1.0 eq.) in Ether (2 mL) was added. The resulting mixture was stirred for another 30 min at same temperature, concentrated under vacuum to get the solid residue which was purified by was purified normal phase silica-gel column chromatography provided title compound as color less solid (0.1 g, 34.1%). LCMS: 243.2 [M+1]⁺

### Step-4: Synthesis of (S)-3-(4-((S)-1-(6-chloro-2-oxo-1,2-dihydroquinolin-3-yl)ethylamino)-6-methyl-1,3,5-triazin-2-yl)-4-ethyloxazolidin-2-one and (S)-3-(4-((R)-1-(6-chloro-2-oxo-1,2-dihydroquinolin-3-yl)ethylamino)-6-methyl-1,3,5-triazin-2-yl)-4-ethyloxazolidin-2-one

In a microwave vial charged with 3-(1-aminoethyl)-6-chloroquinolin-2(1H)-one hydrochloride (0.12 g, 0.54 mmol, 1.0 eq.), (S)-3-(4-chloro-6-methyl-1,3,5-triazin-2-yl)-4-ethyloxazolidin-2-one (0.13 g, 0.54 mmol, 1.0 eq.) and N, N-Diisopropylethylamine (0.13 mL, 0.81 mmol, 1.5 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solids. Compound 1.38 (0.013 g, 6%), UPLC-MS (Method 6): Rt 3.85, m/z 429.1 [M+1]⁺; ¹H NMR (400 MHz, DMSO-d₆) δ ppm 11.99 (br. s., 2H) 8.41-8.49 (m, 1H) 8.26 (d, J=8.33 Hz, 1H) 7.74-7.80 (m, 2H) 7.64 (s, 1H) 7.44-7.52 (m, 2H) 7.27-7.34 (m, 1H) 5.28-5.37 (m, 1H) 5.03-5.12 (m, 1H) 4.59 (br. s., 1H) 4.42 (t, J=8.55 Hz, 1H) 4.29-4.37 (m, 2H) 4.17 (d, J=5.26 Hz, 1H) 4.04 (d, J=5.70 Hz, 1H) 2.25-2.34 (m, 3H) 2.22 (s, 1H) 1.73-1.84 (m, 1H) 1.41 (d, J=7.02 Hz, 4H) 1.19-1.35 (m, 3H) 0.84 (t, J=7.24 Hz, 1H) 0.51 (t, J=7.45 Hz, 3H), Compound 1.39 (0.016 g 7%), UPLC-MS (Method 4): Rt 2.32, m/z 429.1 [M+1]⁺; ¹H NMR (400 MHz, DMSO-d₆) δ ppm 11.98 (br. s., 1H) 8.41 (d, J=7.45 Hz, 1H) 8.24 (d, J=7.89 Hz, 1H) 7.72-7.81 (m, 2H) 7.45-7.54 (m, 1H) 7.26-7.36 (m, 1H) 5.14-5.21 (m, 1H) 4.58 (br. s., 1H) 4.24-4.36 (m, 2H) 4.17 (dd, J=8.55, 2.85 Hz, 1H) 4.06-4.12 (m, 1H) 2.20-2.29 (m, 3H) 1.78-1.91 (m, 2H) 1.63-1.73 (m, 1H) 1.40 (d, J=7.02 Hz, 3H) 0.79-0.90 (m, 3H)

### Example-28: Synthesis of (S)-4-ethyl-3-(4-methyl-6-((S)-1-(1-(4-(trifluoromethyl)phenyl)-1H-imidazol-4-yl)ethylamino)-1,3,5-triazin-2-yl)oxazolidin-2-one (Compound 1.40)

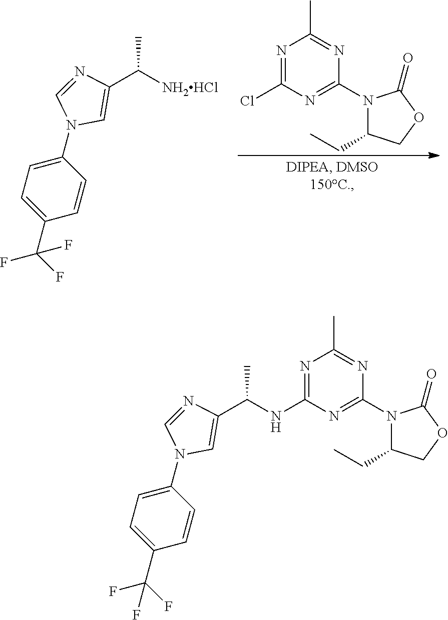

In a microwave vial charged with (S)-1-(1-(4-(trifluoromethyl)phenyl)-1H-imidazol-4-yl)ethanamine hydrochloride (0.1 g, 0.39 mmol, 1.0 eq.), (S)-3-(4-chloro-6-methyl-1,3,5-triazin-2-yl)-4-ethyloxazolidin-2-one (0.10 g, 0.43 mmol, 1.1 eq.) and N, N-Diisopropylethylamine (0.13 mL, 0.78 mmol, 2.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.007 g, 4%), UPLC-MS (Method 6): Rt 3.93, m/z 462.4 [M+1]⁺; ¹H NMR (400 MHz, DMSO-d₆) δ ppm 8.52 (br. s., 1H) 8.35 (s, 1H) 8.19 (d, J=7.89 Hz, 1H) 8.05 (d, J=9.21 Hz, 1H) 7.87 (s, 4H) 7.76 (s, 1H) 7.68 (s, 1H) 5.27-5.32 (m, 1H) 5.12-5.20 (m, 1H) 4.54 (br. s., 1H) 4.40 (t, J=8.33 Hz, 1H) 4.16 (d, J=8.33 Hz, 1H) 2.23-2.32 (m, 3H) 1.70-1.89 (m, 3H) 1.49 (d, J=6.58 Hz, 3H) 0.84 (t, J=7.24 Hz, 3H)

### Example-29: Synthesis of (S)-4-ethyl-3-(4-methoxy-6-((S)-1-(1-(4-(trifluoromethyl)phenyl)-1H-imidazol-4-yl)ethylamino)-1,3,5-triazin-2-yl)oxazolidin-2-one (Compound 1.41)

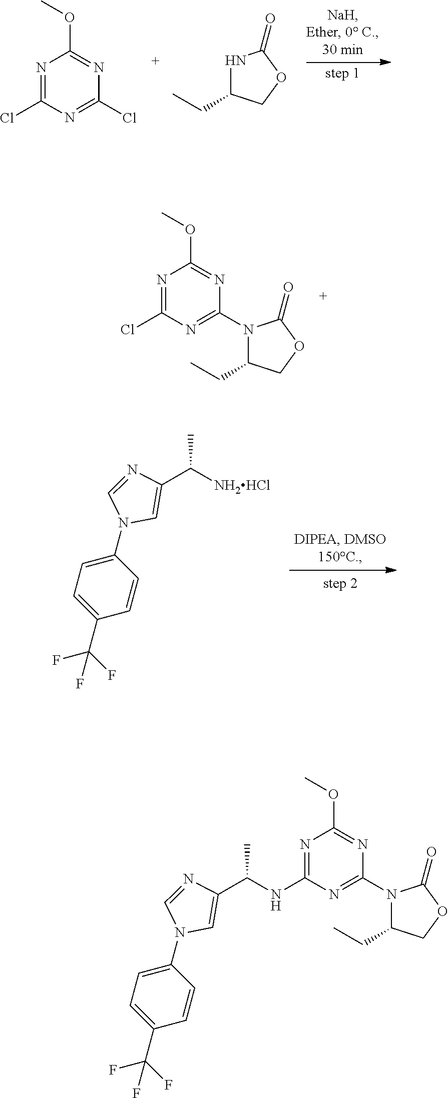

### Step-1: Synthesis of (S)-3-(4-chloro-6-methoxy-1,3,5-triazin-2-yl)-4-ethyloxazolidin-2-one

To a stirred solution of Sodium hydride (0.26 g, 2.6 mmol, 1.2 eq.) in Ether (5 mL) was added (S)-4-ethyloxazolidin-2-one (0.57 g, 5.02 mmol, 0.9 eq.) in Ether (3 mL) at 0° C. The resulting solution stirred for 15 min, following this 2,4-dichloro-6-methoxy-1,3,5-triazine (1.0 g, 5.58 mmol, 1.0 eq.) in Ether (2 mL) was added. The resulting mixture was stirred for another 30 min at same temperature. The reaction mixture concentrated under vacuum to get the solid residue which was purified by was purified normal phase silica-gel column chromatography provided title compound as color less liquid (0.5 g, 35.2%). LCMS: 259.1 [M+1]⁺; ¹H NMR (400 MHz, DMSO-d₆) δ ppm 4.55-4.66 (m, 1H) 4.47 (t, J=8.33 Hz, 1H) 4.27 (dd, J=8.77, 2.63 Hz, 1H) 3.94-4.04 (m, 3H) 1.75-1.89 (m, 2H) 0.87 (t, J=7.45 Hz, 3H)

### Step-2: Synthesis of (S)-4-ethyl-3-(4-methoxy-6-((S)-1-(1-(4-(trifluoromethyl)phenyl)-1H-imidazol-4-yl)ethylamino)-1,3,5-triazin-2-yl)oxazolidin-2-one

In a microwave vial charged with (S)-1-(1-(4-(trifluoromethyl)phenyl)-1H-imidazol-4-yl)ethanamine hydrochloride (0.1 g, 0.39 mmol, 1.0 eq.), (S)-3-(4-chloro-6-methoxy-1,3,5-triazin-2-yl)-4-ethyloxazolidin-2-one (0.11 g, 0.43 mmol, 1.1 eq.) and N, N-Diisopropylethylamine (0.13 mL, 0.78 mmol, 2.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.034 g, 18%), UPLC-MS (Method 4): Rt 2.25, m/z 478.4 [M+1]⁺; ¹H NMR (400 MHz, DMSO-d₆) δ ppm 8.36 (d, J=6.58 Hz, 1H) 8.20 (dd, J=19.07, 8.11 Hz, 1H) 7.84-7.92 (m, 4H) 7.69 (s, 1H) 5.11-5.24 (m, 1H) 4.47-4.58 (m, 1H) 4.34-4.44 (m, 1H) 4.14 (ddd, J=16.55, 8.66, 2.85 Hz, 1H) 3.84 (s, 3H) 1.75-1.87 (m, 1H) 1.59-1.68 (m, 1H) 1.50 (d, J=7.02 Hz, 3H) 1.23-1.29 (m, 1H) 0.85 (t, J=7.45 Hz, 2H) 0.71 (t, J=7.45 Hz, 2H)

### Example-30: Synthesis of (S)-3-(4-((S)-1-(6-chloro-7-methoxy-2-oxo-1,2-dihydroquinolin-3-yl)ethylamino)-6-methyl-1,3,5-triazin-2-yl)-4-ethyloxazolidin-2-one (Compound 1.42) and (S)-3-(4-((R)-1-(6-chloro-7-methoxy-2-oxo-1,2-dihydroquinolin-3-yl)ethylamino)-6-methyl-1,3,5-triazin-2-yl)-4-ethyloxazolidin-2-one (Compound 1.43)

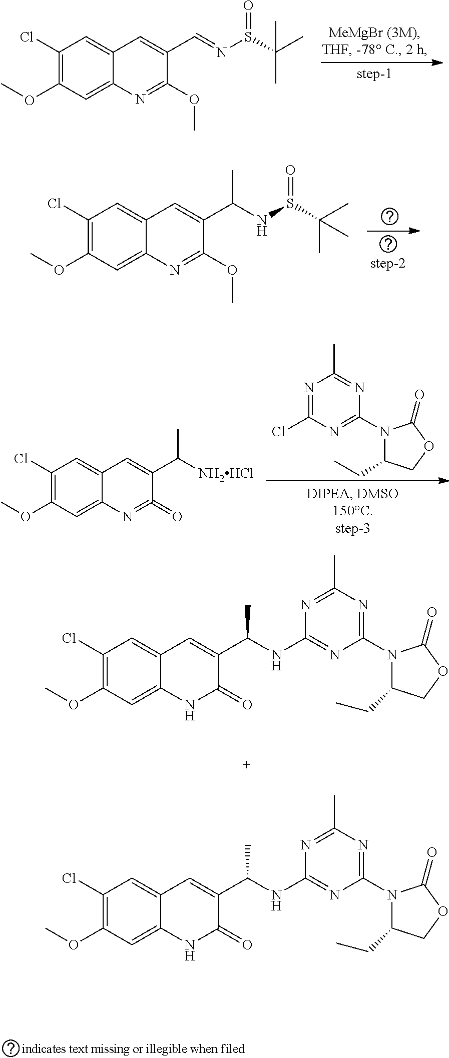

### Step-1: Synthesis of (S)—N-(1-(6-chloro-2,7-dimethoxyquinolin-3-yl)ethyl)-2-methylpropane-2-sulfinamide

To a stirred solution of (S,E)-N-((6-chloro-2-methoxyquinolin-3-yl)methylene)-2-methylpropane-2-sulfinamide (1.0 g, 1.306 mmol, 1.0 eq.) in THF (50 mL) was added drop wise 3 molar methylmagnesium bromide (1.45 mL, 5.2 mmol, 1.5 eq.) at −78° C. The resulting mixture was stirred for 2 h at same temperature. The reaction was then quenched by careful addition of saturated NH₄Cl (10 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound as semi solid (0.3 g, 62.3%). LCMS: 371.2 [M+1]⁺; ¹H NMR (400 MHz, DMSO-d₆) δ ppm 8.14 (s, 1H) 7.98 (s, 1H) 7.67 (d, J=9.21 Hz, 1H) 7.34 (s, 1H) 4.74-4.80 (m, 1H) 3.96-4.05 (m, 6H) 1.41 (d, J=7.02 Hz, 3H) 1.21-1.25 (m, 9H)

### Step-2: Synthesis of 3-(1-aminoethyl)-6-chloro-7-methoxyquinolin-2(1H)-one hydrochloride

To a stirred solution of (S)—N-(1-(6-chloro-2,7-dimethoxyquinolin-3-yl)ethyl)-2-methylpropane-2-sulfinamide (0.3 g, 0.84 mmol, 1.0 eq.) in dioxane (10 mL) was added 4N HCl in dioxane (0.4 mL, 2.52 mmol, 3.0 eq.) at RT. The resulting mixture was heated to reflux for 3 h. Following this, the reaction mixture was evaporated under reduced pressure to get title compound which is used to next step without further purification (0.23 g crude). LCMS: 253.1 [M+1]⁺

### Step-3: Synthesis of (S)-3-(4-((S)-1-(6-chloro-7-methoxy-2-oxo-1,2-dihydroquinolin-3-yl)ethylamino)-6-methyl-1,3,5-triazin-2-yl)-4-ethyloxazolidin-2-one and (S)-3-(4-((R)-1-(6-chloro-7-methoxy-2-oxo-1,2-dihydroquinolin-3-yl)ethylamino)-6-methyl-1,3,5-triazin-2-yl)-4-ethyloxazolidin-2-one

In a microwave vial charged with 3-(1-aminoethyl)-6-chloro-7-methoxyquinolin-2(1H)-one hydrochloride (0.13 g, 0.51 mmol, 1.0 eq.), (S)-3-(4-chloro-6-methoxy-1,3,5-triazin-2-yl)-4-ethyloxazolidin-2-one (0.15 g, 0.61 mmol, 1.2 eq.) and N, N-Diisopropylethylamine (0.17 mL, 1.02 mmol, 2.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid. Compound 1.42 (0.022 g, 9%), UPLC-MS (Method 1): Rt 4.84, m/z 459.4 [M+1]⁺; ¹H NMR (400 MHz, DMSO-d₆) δ ppm 11.80 (br. s., 1H) 8.40 (d, J=6.58 Hz, 1H) 8.20 (d, J=8.33 Hz, 1H) 7.73-7.79 (m, 1H) 7.54 (s, 1H) 6.88-6.97 (m, 2H) 5.23-5.32 (m, 1H) 4.99-5.08 (m, 1H) 4.57 (br. s., 1H) 4.28-4.43 (m, 3H) 4.15 (dd, J=8.33, 3.07 Hz, 1H) 4.03 (d, J=6.14 Hz, 1H) 3.82-3.88 (m, 4H) 2.26 (s, 3H) 2.21 (s, 1H) 1.71-1.81 (m, 1H) 1.37 (d, J=6.58 Hz, 4H) 1.21-1.34 (m, 3H) 0.82 (t, J=7.45 Hz, 1H) 0.52 (t, J=7.45 Hz, 3H). Compound 1.43 (0.019 g, 8%), UPLC-MS (Method 1): Rt 5.23, m/z 459.4 [M+1]⁺; ¹H NMR (400 MHz, DMSO-d₆) δ ppm 11.83 (br. s., 1H) 8.37 (d, J=7.89 Hz, 1H) 7.74-7.79 (m, 1H) 7.65-7.72 (m, 1H) 6.91-6.98 (m, 1H) 5.24-5.35 (m, 1H) 5.15 (quin, J=7.13 Hz, 1H) 4.57 (t, J=7.45 Hz, 1H) 4.25-4.46 (m, 2H) 4.17 (dd, J=8.77, 2.63 Hz, 1H) 4.10 (dd, J=7.89, 1.75 Hz, 1H) 3.82-3.92 (m, 3H) 2.16-2.31 (m, 3H) 1.63-1.89 (m, 2H) 1.38 (d, J=7.02 Hz, 3H) 0.84 (q, J=7.31 Hz, 3H).

### Example-31: Synthesis of (S)-4-ethyl-3-(4-methyl-6-((S)-1-(4-phenoxyphenyl)ethylamino)-1,3,5-triazin-2-yl)oxazolidin-2-one (Compound 1.44)

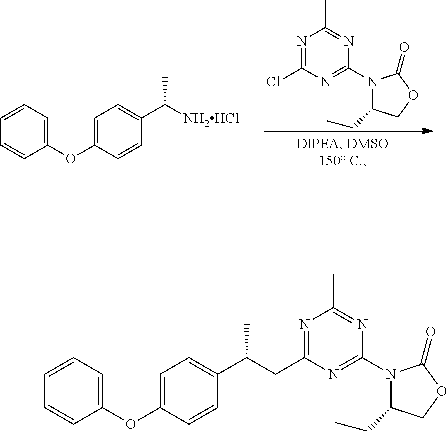

In a microwave vial charged with (S)-1-(4-phenoxyphenyl)ethanamine hydrochloride (0.13 g, 0.51 mmol, 1.0 eq.), (S)-3-(4-chloro-6-methoxy-1,3,5-triazin-2-yl)-4-ethyloxazolidin-2-one (0.15 g, 0.61 mmol, 1.2 eq.) and N, N-Diisopropylethylamine (0.17 mL, 1.02 mmol, 2.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.03 g, 14%), UPLC-MS (Method 4): Rt 2.60, m/z 420.4 [M+1]⁺; ¹H NMR (400 MHz, DMSO-d₆) δ ppm 11.80 (br. s., 1H) 8.40 (d, J=6.58 Hz, 1H) 8.20 (d, J=8.33 Hz, 1H) 7.73-7.79 (m, 1H) 7.54 (s, 1H) 6.88-6.97 (m, 2H) 5.23-5.32 (m, 1H) 4.99-5.08 (m, 1H) 4.57 (br. s., 1H) 4.28-4.43 (m, 3H) 4.15 (dd, J=8.33, 3.07 Hz, 1H) 4.03 (d, J=6.14 Hz, 1H) 3.82-3.88 (m, 4H) 2.26 (s, 3H) 2.21 (s, 1H) 1.71-1.81 (m, 1H) 1.37 (d, J=6.58 Hz, 4H) 1.21-1.34 (m, 3H) 0.82 (t, J=7.45 Hz, 1H) 0.52 (t, J=7.45 Hz, 3H).

### Example-32: Synthesis of (S)-4-ethyl-3-(4-methyl-6-((S)-1-(2-oxo-1,2-dihydroquinolin-3-yl)ethylamino)-1,3,5-triazin-2-yl)oxazolidin-2-one (Compound 1.45) and (S)-4-ethyl-3-(4-methyl-6-((R)-1-(2-oxo-1,2-dihydroquinolin-3-yl)ethylamino)-1,3,5-triazin-2-yl) oxazolidin-2-one (Compound 1.46)

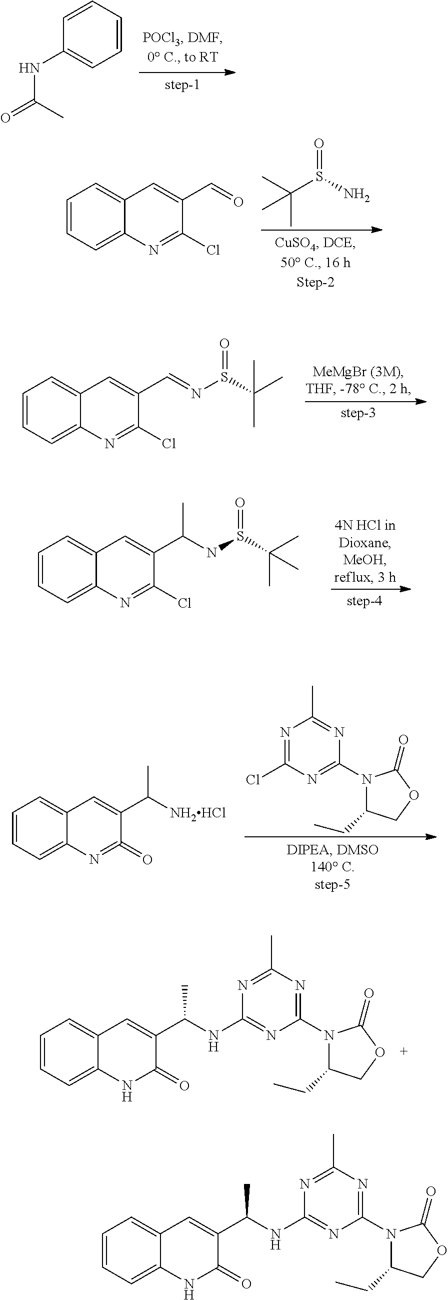

### Step-1: Synthesis of 2-chloroquinoline-3-carbaldehyde

DMF (3.87 mL, 50.0 mmol, 2.5 eq.) was cooled to 0° C. and added POCl₃ (13.0 mL, 140.0 mmol, 7.0 eq.) drop wise over 5 min. To the above solution added N-phenylacetamide (2.72 g, 20.0 mmol, 1.0 eq.) dissolved in DMF (2 mL). The resulting mixture heated at 100° C. for 16 h. Following this, reaction mixture diluted with ice cold water (100 mL) and stirred for 1 h. Filtered the solid and under vacuum to get title compound as half white solid (1.5 g, 39%). ¹H NMR (400 MHz, DMSO-d₆) δ ppm 10.39 (s, 1H) 9.00 (s, 1H) 8.29 (d, J=7.89 Hz, 1H) 7.96-8.08 (m, 2H) 7.74-7.82 (m, 1H)

### Step-2: Synthesis of (S,E)-N-((2-chloroquinolin-3-yl)methylene)-2-methylpropane-2-sulfinamide

To a stirred solution of 2-chloroquinoline-3-carbaldehyde (1.5 g, 7.8 mmol, 1.0 eq.) and Copper (II) sulfate (2.48 g, 15.60 mmol, 2.0 eq.) in dichloroethane (15 mL) was added (S)-2-methylpropane-2-sulfinamide (1.85 g, 15.60 mmol, 2.0 eq.) at RT. The resulting mixture was heated at 50° C. for 16 h. Following this, reaction was allowed to cool to room temperature, filtered through celite pad, the celite pad washed with dichloromethane (30 mL). The combined filtrate dried over anhydrous Na₂SO₄ and concentrated under vacuum to get the solid residue which was purified by normal phase silica-gel column chromatography to get the title compound (1.2 g 52.1%). LCMS: 296.1 [M+1]⁺; ¹H NMR (400 MHz, DMSO-d₆) δ ppm 9.13 (s, 1H) 8.91 (s, 1H) 8.28 (d, J=7.89 Hz, 1H) 8.01-8.07 (m, 1H) 7.96 (dd, J=6.80, 1.53 Hz, 1H) 7.72-7.79 (m, 1H) 1.25 (s, 10H).

### Step-3: Synthesis of (S)—N-(1-(2-chloroquinolin-3-yl)ethyl)-2-methylpropane-2-sulfinamide

To a stirred solution of (S,E)-N-((2-chloroquinolin-3-yl)methylene)-2-methylpropane-2-sulfinamide (1.2 g, 4.06 mmol, 1.0 eq.) in THF (25 mL) was added drop wise 3 molar methylmagnesium bromide (4.8 mL, 14.24 mmol, 3.5 eq.) at −78° C. The resulting mixture was stirred for 2 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.7 g 55.4%). LCMS: 312.1 [M+1]⁺. ¹H NMR (400 MHz, DMSO-d₆) δ ppm 8.54 (d, J=19.73 Hz, 1H) 8.03 (br. s., 1H) 7.96 (br. s., 1H) 7.81 (br. s., 1H) 7.67 (br. s., 1H) 5.75 (br. s., 1H) 5.64 (br. s., 1H) 4.85 (br. s., 1H) 1.62 (br. s., 1H) 1.52 (br. s., 2H) 1.23 (br. s., 1H) 1.12 (br. s., 7H)

### Step-4: Synthesis of 3-(1-aminoethyl)quinolin-2(1H)-one hydrochloride

To a stirred solution of (S)—N-(1-(2-chloroquinolin-3-yl)ethyl)-2-methylpropane-2-sulfinamide (0.7 g, 2.25 mmol, 1.0 eq.) in MeOH (5 mL) was added 4N HCl in dioxane (4.0 mL, 6.23 mmol, 5.0 eq.) at RT. The resulting mixture was heated to reflux for 3 h. Following this, the reaction mixture was evaporated under reduced pressure to get title compound which is used to next step without further purification (0.3 g 59.2%). LCMS: 226.1 [M+1]⁺.

### Step-5: Synthesis of (S)-4-ethyl-3-(4-methyl-6-((S)-1-(2-oxo-1,2-dihydroquinolin-3-yl)ethylamino)-1,3,5-triazin-2-yl)oxazolidin-2-one and (S)-4-ethyl-3-(4-methyl-6-((R)-1-(2-oxo-1,2-dihydroquinolin-3-yl)ethylamino)-1,3,5-triazin-2-yl)oxazolidin-2-one

In a microwave vial charged with 3-(1-aminoethyl)quinolin-2(1H)-one hydrochloride (0.08 g, 0.35 mmol, 1.0 eq.), (S)-3-(4-chloro-1,3,5-triazin-2-yl)-4-isopropyloxazolidin-2-one (0.086 g, 0.35 mmol, 1.0 eq.) and N, N-Diisopropylethylamine (0.11 mL, 1.05 mmol, 3.0 eq.), in DMSO (2 mL). The resulting mixture was heated at 140° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid. Compound 1.45 (0.008 g, 6%), UPLC-MS (Method 2): Rt 2.37, m/z 395.4 [M+1]⁺; ¹H NMR (400 MHz, DMSO-d₆) δ ppm 11.83 (br. s., 1H) 8.44 (d, J=7.02 Hz, 1H) 8.26 (d, J=8.33 Hz, 1H) 7.58-7.67 (m, 2H) 7.40-7.50 (m, 2H) 7.27-7.33 (m, 1H) 7.08-7.21 (m, 2H) 5.33-5.39 (m, 1H) 5.12 (t, J=7.02 Hz, 1H) 4.60 (br. s., 1H) 4.30-4.45 (m, 3H) 4.17 (dd, J=8.33, 3.07 Hz, 1H) 4.05 (dd, J=8.11, 2.41 Hz, 1H) 2.28 (s, 2H) 2.23 (s, 1H) 1.73-1.85 (m, 1H) 1.41 (d, J=7.02 Hz, 4H) 1.34 (d, J=7.45 Hz, 1H) 1.21-1.30 (m, 2H) 0.84 (t, J=7.45 Hz, 1H) 0.53 (t, J=7.24 Hz, 3H). Compound 1.46 (0.006 g, 5%), UPLC-MS (Method 2): Rt 2.43, m/z 395.4 [M+1]⁺; ¹H NMR (400 MHz, DMSO-d₆) δ ppm 11.84 (br. s., 1H) 8.40 (d, J=7.45 Hz, 1H) 8.25 (d, J=8.33 Hz, 1H) 7.76-7.82 (m, 1H) 7.61 (d, J=7.89 Hz, 1H) 7.40-7.49 (m, 1H) 7.25-7.34 (m, 1H) 7.11-7.20 (m, 1H) 5.32-5.39 (m, 1H) 5.18-5.25 (m, 1H) 4.58 (br. s., 1H) 4.41 (t, J=8.33 Hz, 1H) 4.27-4.36 (m, 1H) 4.09 (d, J=5.70 Hz, 1H) 2.21-2.30 (m, 3H) 1.76-1.91 (m, 2H) 1.71 (dd, J=14.03, 7.02 Hz, 1H) 1.40 (d, J=7.02 Hz, 2H) 0.80-0.91 (m, 3H).

### Example-33: Synthesis of (S)-3-(4-((S)-1-(6-bromoimidazo[1,2-a]pyridin-2-yl)ethylamino)-6-methyl-1,3,5-triazin-2-yl)-4-ethyloxazolidin-2-one (Compound 1.47) and (S)-3-(4-((R)-1-(6-bromoimidazo[1,2-a]pyridin-2-yl)ethylamino)-6-methyl-1,3,5-triazin-2-yl)-4-ethyloxazolidin-2-one (Compound 1.48)

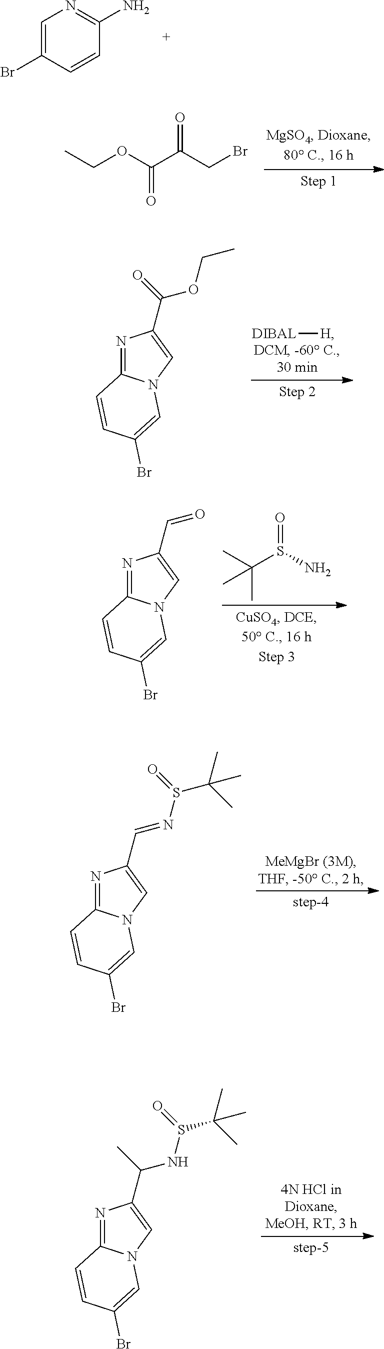

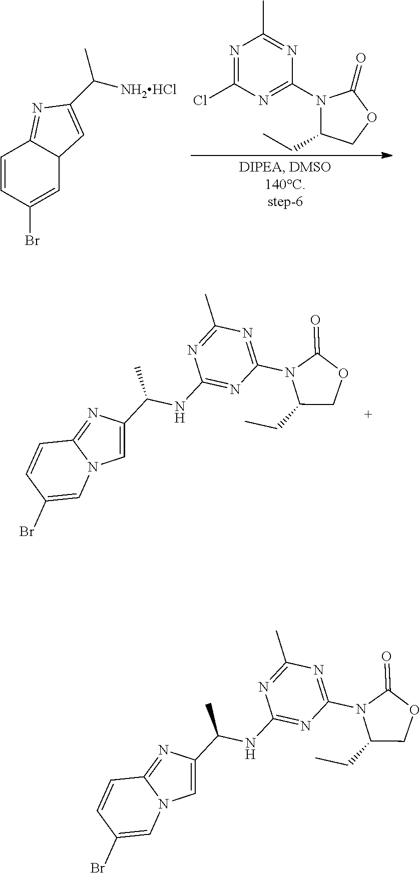

### Step-1: Synthesis of ethyl 6-bromoimidazo[1,2-a]pyridine-2-carboxylate

To a stirred solution of 5-bromopyridin-2-amine (10.0 g, 56.0 mmol, 1.0 eq.) and ethyl 3-bromo-2-oxopropanoate (12.6 g, 64.0 mmol, 1.1 eq.) in Dioxane (200 mL) was added MgSO₄ (20.0 g, 150.0 mmol, 3.0 eq.) at RT. The resulting mixture heated to 80° C. for 16 h. Following this, reaction mixture cooled to RT filtered the solid and under vacuum and filtrate concentrated to get crude. The crude purified by normal phase silica-gel column to get title compound (12 g, 79%). LCMS: 269.9 [M+1]⁺; ¹H NMR (400 MHz, DMSO-d₆) δ ppm 8.91 (d, J=1.32 Hz, 1H) 8.47 (s, 1H) 7.62 (d, J=9.65 Hz, 1H) 7.47 (dd, J=9.65, 1.75 Hz, 1H) 4.31 (q, J=7.16 Hz, 2H) 1.31 (t, J=7.02 Hz, 3H)

### Step-2: Synthesis of 6-bromoimidazo[1,2-a]pyridine-2-carbaldehyde

To a stirred solution of ethyl 6-bromoimidazo[1,2-a]pyridine-2-carboxylate (3.0 g, 11.1 mmol, 1.0 eq.) in DCM (100 mL) was added Diisobutylaluminium hydride (44.6 mL, 44.6 mmol, 4.0 eq.) at −60° C. over 5 min. The resulting mixture stirred for 30 min. After completion of starting material, the reaction mixture quenched with MeOH (10 mL), concentrated to give crude. The crude re dissolved in water (20 mL) and extracted with ethyl acetate (3×20 mL). The combined organic layers were washed with brine (50 mL), dried over anhydrous Na₂SO₄, filtered and concentrated under vacuum to get the solid residue which was purified by was purified normal phase silica-gel column to get the title compound as off-white solid (1.3 g 51.8%). LCMS: 226.2 [M+1]⁺; ¹H NMR (400 MHz, DMSO-d₆) δ ppm 10.04 (s, 1H) 8.99 (d, J=0.88 Hz, 1H) 8.57 (s, 1H) 7.67 (d, J=9.65 Hz, 1H) 7.51 (dd, J=9.65, 1.75 Hz, 1H).

### Step-3: Synthesis of (E)-N-((6-bromoimidazo[1,2-a]pyridin-2-yl)methylene)-2-methylpropane-2-sulfinamide

To a stirred solution of 6-bromoimidazo[1,2-a]pyridine-2-carbaldehyde (1.4 g, 6.0 mmol, 1.0 eq.) and Copper (II) sulfate (2.0 g, 12.0 mmol, 2.0 eq.) in dichloroethane (50 mL) was added (S)-2-methylpropane-2-sulfinamide (1.50 g, 12.0 mmol, 2.0 eq.) at RT. The resulting mixture was heated at 50° C. for 16 h. Following this, reaction was allowed to cool to room temperature, filtered through celite pad, the celite pad washed with dichloromethane (30 mL). The combined filtrate dried over anhydrous Na₂SO₄ and concentrated under vacuum to get the solid residue which was purified by normal phase silica-gel column chromatography to get the title compound (0.2 g 10.1%). LCMS: 327.9 [M+1]⁺; ¹H NMR (400 MHz, DMSO-d₆) δ ppm 9.03 (br. s., 1H) 8.64 (br. s., 1H) 7.68 (br. s., 1H) 7.54 (br. s., 1H) 1.17 (s, 6H) 1.09 (s, 9H).

### Step-4: Synthesis of (S)—N-(1-(6-bromoimidazo[1,2-a]pyridin-2-yl)ethyl)-2-methylpropane-2-sulfinamide

To a stirred solution of (E)-N-((6-bromoimidazo[1,2-a]pyridin-2-yl)methylene)-2-methylpropane-2-sulfinamide (0.2 g, 0.7 mmol, 1.0 eq.) in THF (10 mL) was added drop wise 3 molar methylmagnesium bromide (0.7 mL, 2.11 mmol, 3.0 eq.) at −50° C. The resulting mixture was stirred for 2 h at same temperature. The reaction was then quenched by careful addition of saturated NH₄Cl (20 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound (0.2 g 83.2%). LCMS: 344.1 [M+1]⁺.

### Step-5: Synthesis of 1-(6-bromoimidazo[1,2-a]pyridin-2-yl)ethanamine hydrochloride

To a stirred solution of (S)—N-(1-(6-bromoimidazo[1,2-a]pyridin-2-yl)ethyl)-2-methylpropane-2-sulfinamide (0.2 g, 0.58 mmol, 1.0 eq.) in MeOH (5 mL) was added 4N HCl in dioxane (0.5 mL, 1.73 mmol, 3.0 eq.) at RT. The resulting mixture stirred for 3 h. Following this, the reaction mixture was evaporated under reduced pressure to get title compound which is used to next step without further purification (0.12 g 86.5%). LCMS: 240.1 [M+1]⁺. ¹H NMR (400 MHz, DMSO-d₆) δ ppm 9.10 (s, 1H) 8.59 (br. s., 2H) 8.06 (s, 1H) 7.66 (d, J=9.65 Hz, 1H) 7.58 (d, J=9.21 Hz, 1H) 4.61 (d, J=4.82 Hz, 1H) 1.58 (d, J=7.02 Hz, 3H)

### Step-6: Synthesis of (S)-3-(4-((S)-1-(6-bromoimidazo[1,2-a]pyridin-2-yl)ethylamino)-6-methyl-1,3,5-triazin-2-yl)-4-ethyloxazolidin-2-one and (S)-3-(4-((R)-1-(6-bromoimidazo[1,2-a]pyridin-2-yl)ethylamino)-6-methyl-1,3,5-triazin-2-yl)-4-ethyloxazolidin-2-one

In a microwave vial charged with 1-(6-bromoimidazo[1,2-a]pyridin-2-yl)ethanamine hydrochloride (0.12 g, 0.5 mmol, 1.0 eq.), (S)-3-(4-chloro-1,3,5-triazin-2-yl)-4-isopropyloxazolidin-2-one (0.14 g, 0.6 mmol, 1.0 eq.) and N, N-Diisopropylethylamine (0.2 mL, 1.05 mmol, 2.0 eq.), in DMSO (5 mL). The resulting mixture was heated at 140° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid. Compound 1.47 (0.006 g, 3%), UPLC-MS (Method 6): Rt 2.77, m/z 446.3 [M+1]⁺; ¹H NMR (400 MHz, DMSO-d₆) δ ppm 8.78-8.88 (m, 2H) 8.45 (d, J=7.89 Hz, 1H) 8.31 (d, J=8.77 Hz, 1H) 7.74 (s, 1H) 7.69 (s, 1H) 7.43-7.53 (m, 2H) 7.25-7.36 (m, 2H) 5.39-5.46 (m, 1H) 5.22-5.30 (m, 1H) 4.57 (br. s., 1H) 4.32-4.49 (m, 3H) 4.17 (dd, J=8.33, 3.07 Hz, 1H) 4.08 (dd, J=8.55, 2.85 Hz, 1H) 2.25-2.29 (m, 4H) 1.79 (dt, J=13.92, 6.85 Hz, 2H) 1.38-1.59 (m, 7H) 0.85 (t, J=7.45 Hz, 2H) 0.57 (t, J=7.45 Hz, 3H). Compound 1.48 (0.004 g, 2%), UPLC-MS (Method 2): Rt 2.19, m/z 446.3 [M+1]⁺; ¹H NMR (400 MHz, DMSO-d₆) δ ppm 8.85 (br. s., 1H) 8.42 (d, J=8.33 Hz, 1H) 8.31 (d, J=8.77 Hz, 1H) 7.70-7.82 (m, 1H) 7.48 (d, J=9.65 Hz, 1H) 7.31 (dd, J=9.65, 1.75 Hz, 1H) 5.37-5.47 (m, 1H) 5.24-5.37 (m, 1H) 4.57 (br. s., 1H) 4.32-4.52 (m, 2H) 4.08-4.22 (m, 2H) 2.27 (s, 2H) 1.70-1.85 (m, 2H) 1.46-1.57 (m, 3H) 0.78-0.88 (m, 3H).

### Example-34: Synthesis of (S)-3-(4-((S)-1-(6-chloro-2-oxo-1,2-dihydro-1,8-naphthyridin-3-yl)ethylamino)-6-methyl-1,3,5-triazin-2-yl)-4-ethyloxazolidin-2-one (Compound 1.49)

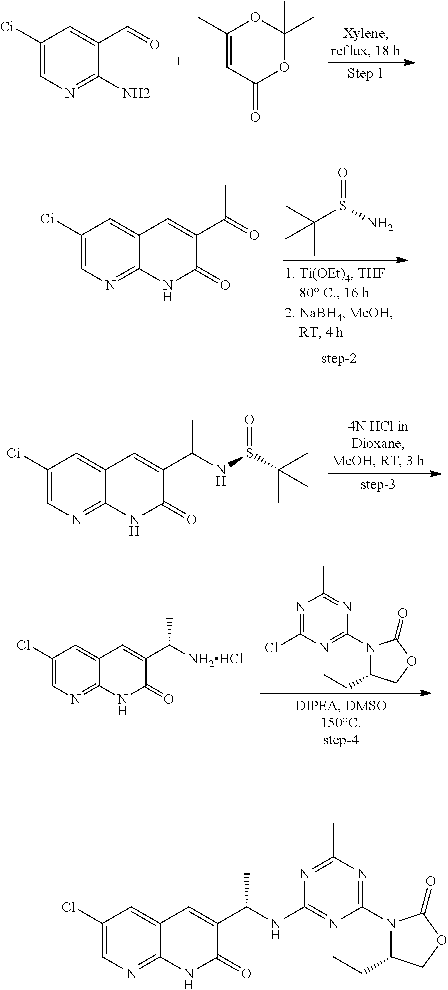

### Step-1: Synthesis of 3-acetyl-6-chloro-1,8-naphthyridin-2(1H)-one

To a stirred solution of 2-amino-5-chloronicotinaldehyde (2.0 g, 12.0 mmol, 1.0 eq.) and 2,2,6-trimethyl-4H-1,3-dioxin-4-one (2.4 g, 16.0 mmol, 1.3 eq.) in xylene (50 mL) heated to reflux for 18 h. Following this, reaction mixture cooled to RT filtered the solid and dried under vacuum to get title compound (2 g, 74.8%). ¹H NMR (400 MHz, DMSO-d₆) δ ppm 12.71 (br. s., 1H) 8.66 (d, J=2.63 Hz, 1H) 8.51 (d, J=2.19 Hz, 1H) 8.42 (s, 1H) 2.57-2.64 (m, 3H).

### Step-2: Synthesis of (S)—N-(1-(6-chloro-2-oxo-1,2-dihydro-1,8-naphthyridin-3-yl)ethyl)-2-methylpropane-2-sulfinamide

To a stirred solution of 3-acetyl-6-chloro-1,8-naphthyridin-2(1H)-one (2.0 g, 9.0 mmol, 1.0 eq.) and Titanium(IV) ethoxide (2.7 g, 22.0 mmol, 2.5 eq.) in THF (50 mL) was added (S)-2-methylpropane-2-sulfinamide (6.2 g, 26.0 mmol, 3.0 eq.) at RT. The resulting mixture was heated at 80° C. for 16 h. Following this, reaction was allowed to cool to room temperature and evaporated to get solid residue. The residue dissolved in MeOH (20 mL) and added Sodium borohydride (0.4 g, 10.8 mmol, 1.2 eq.) at RT. The resulting mixture stirred for 4 h. After completion of starting material, filtered through celite pad, the celite pad washed with dichloromethane (30 mL). The combined filtrate dried over anhydrous Na₂SO₄ and concentrated under vacuum to get the solid residue which was purified by normal phase silica-gel column chromatography to get the title compound (0.7 g 23.5%). LCMS: 327.9 [M+1]⁺; ¹H NMR (400 MHz, DMSO-d₆) δ ppm 12.71 (br. s., 1H) 8.62 (d, J=2.19 Hz, 1H) 8.51 (d, J=2.19 Hz, 1H) 8.14 (s, 1H) 5.31 (s, 1H) 1.13 (s, 3H) 1.07 (s, 9H).

### Step-3: Synthesis of (S)-3-(1-aminoethyl)-6-chloro-1,8-naphthyridin-2(1H)-one hydrochloride

To a stirred solution of (S)—N-(1-(6-chloro-2-oxo-1,2-dihydro-1,8-naphthyridin-3-yl)ethyl)-2-methylpropane-2-sulfinamide (0.2 g, 0.61 mmol, 1.0 eq.) in MeOH (10 mL) was added 4N HCl in dioxane (0.6 mL, 1.83 mmol, 3.0 eq.) at RT. The resulting mixture stirred for 3 h. Following this, the reaction mixture was evaporated under reduced pressure to get title compound which is used to next step without further purification (0.12 g 88.2%). LCMS: 224.1 [M+1]⁺.

### Step-4: Synthesis of (S)-3-(4-((S)-1-(6-chloro-2-oxo-1,2-dihydro-1,8-naphthyridin-3-yl)ethylamino)-6-methyl-1,3,5-triazin-2-yl)-4-ethyloxazolidin-2-one

In a microwave vial charged with (S)-3-(1-aminoethyl)-6-chloro-1,8-naphthyridin-2(1H)-one hydrochloride (0.1 g, 0.44 mmol, 1.0 eq.), (S)-3-(4-chloro-1,3,5-triazin-2-yl)-4-isopropyloxazolidin-2-one (0.119 g, 0.49 mmol, 1.1 eq.) and N, N-Diisopropylethylamine (0.2 mL, 0.89 mmol, 2.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.039 g, 20%), UPLC-MS (Method 6): Rt 3.44, m/z 430.4 [M+1]⁺; ¹H NMR (400 MHz, DMSO-d₆) δ ppm 12.41 (br. s., 1H) 8.46-8.52 (m, 1H) 8.28-8.35 (m, 1H) 7.72-7.79 (m, 1H) 5.27-5.35 (m, 1H) 5.10-5.20 (m, 1H) 4.58 (br. s., 1H) 4.40 (t, J=8.33 Hz, 1H) 4.15-4.34 (m, 2H) 4.08 (dd, J=8.11, 2.41 Hz, 1H) 2.27 (s, 2H) 2.22 (s, 1H) 1.62-1.88 (m, 3H) 1.40 (d, J=6.58 Hz, 3H) 0.76-0.90 (m, 3H).

### Example-35: Synthesis of (S)-3-(4-((S)-1-(1-(4-chlorophenyl)-1H-imidazol-4-yl)ethylamino)-6-methyl-1,3,5-triazin-2-yl)-4-ethyloxazolidin-2-one (Compound 1.50) and (S)-3-(4-((R)-1-(1-(4-chlorophenyl)-H-imidazol-4-yl)ethylamino)-6-methyl-1,3,5-triazin-2-yl)-4-ethyloxazolidin-2-one (Compound 1.51)

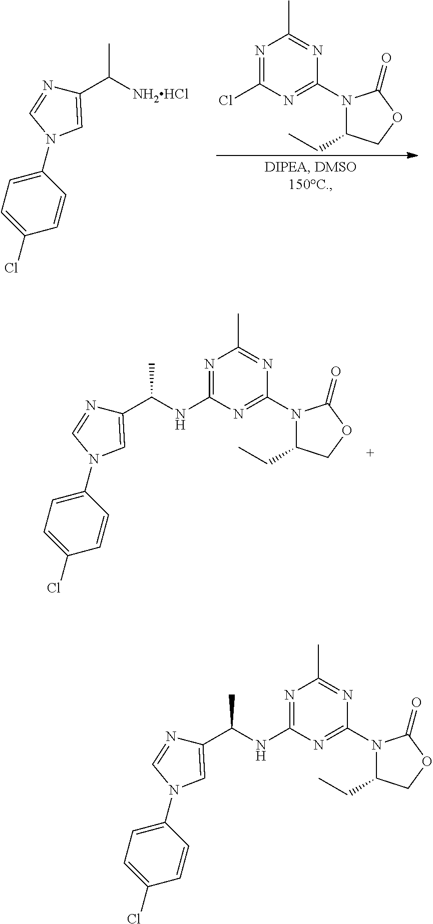

In a microwave vial charged with 1-(1-(4-chlorophenyl)-1H-imidazol-4-yl)ethanamine hydrochloride (0.10 g, 0.45 mmol, 1.0 eq.), (S)-3-(4-chloro-1,3,5-triazin-2-yl)-4-isopropyloxazolidin-2-one (0.11 g, 0.45 mmol, 1.0 eq.) and N, N-Diisopropylethylamine (0.15 mL, 0.9 mmol, 2.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid. Compound 1.50 (0.008 g, 4%), UPLC-MS (Method 2): Rt 2.46, m/z 428.4 [M+1]⁺; ¹H NMR (400 MHz, DMSO-d₆) δ ppm 8.20 (d, J=8.77 Hz, 2H) 8.04 (d, J=8.77 Hz, 1H) 7.62-7.71 (m, 3H) 7.51-7.59 (m, 4H) 5.28 (br. s., 1H) 5.06-5.15 (m, 1H) 4.53 (br. s., 2H) 4.35-4.45 (m, 2H) 4.08-4.18 (m, 2H) 2.24-2.30 (m, 4H) 1.88 (s, 1H) 1.48 (d, J=7.02 Hz, 4H) 0.84 (t, J=7.24 Hz, 1H) 0.71 (t, J=7.45 Hz, 3H). Compound 1.51 (0.005 g, 3%), UPLC-MS (Method 2): Rt 2.48, m/z 428.4 [M+1]⁺; ¹H NMR (400 MHz, DMSO-d₆) δ ppm 8.21 (s, 1H) 8.17 (d, J=8.33 Hz, 1H) 8.03 (d, J=8.77 Hz, 1H) 7.63-7.70 (m, 3H) 7.53-7.59 (m, 2H) 5.25-5.31 (m, 1H) 5.10-5.18 (m, 1H) 4.54 (br. s., 1H) 4.40 (t, J=8.11 Hz, 1H) 4.16 (dd, J=8.77, 3.07 Hz, 1H) 2.23-2.31 (m, 3H) 1.87 (s, 1H) 1.75-1.84 (m, 2H) 1.47 (d, J=7.02 Hz, 3H) 0.84 (t, J=7.24 Hz, 3H).

### Example-36: Synthesis of (S)-3-(4-((S)-1-(6-chloro-2-oxo-1,2-dihydro-1,8-naphthyridin-3-yl)ethylamino)-6-methyl-1,3,5-triazin-2-yl)-4-ethyloxazolidin-2-one (Compound 1.52)

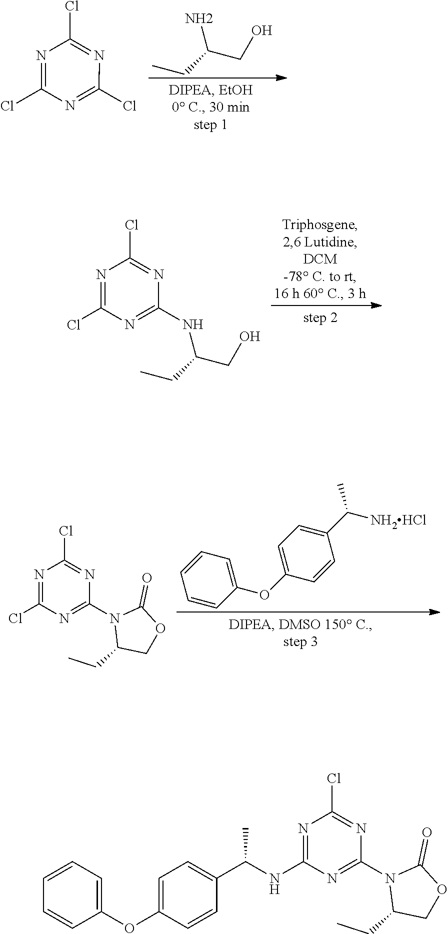

### Step-1: Synthesis of (S)-2-(4,6-dichloro-1,3,5-triazin-2-ylamino)butan-1-ol

To a stirred solution of 2,4,6-trichloro-1,3,5-triazine (1.0 g, 5.4 mmol, 1.0 eq.), (S)-2-aminobutan-1-ol (0.52 g, 5.9 mmol, 1.1 eq.) in EtOH (10 mL) was added DIPEA (1.39 g, 10.0 mmol, 2.0 eq.) at RT. The resulting mixture was stirred for 30 min. Following this, concentrated under vacuum to get the solid residue which was purified by was purified normal phase silica-gel column to get the title compound as brown color semi solid (0.6 g 47%). LCMS: 236.9 [M+1]⁺; ¹H NMR (400 MHz, DMSO-d₆) δ ppm 8.94 (d, J=8.77 Hz, 1 H) 3.77-3.88 (m, 1H) 3.33-3.44 (m, 3H) 1.56-1.69 (m, 1H) 1.32-1.46 (m, 1H) 0.85 (t, J=7.45 Hz, 3H).

### Step-2: Synthesis of (S)-3-(4,6-dichloro-1,3,5-triazin-2-yl)-4-ethyloxazolidin-2-one

To a stirred solution of (S)-2-(4,6-dichloro-1,3,5-triazin-2-ylamino)butan-1-ol (0.6 g, 2.5 mmol, 1.0 eq.), 2,6 lutidine (0.6 mL, 11.2 mmol, 4.5 eq.) in DCM (10 mL) was added Triphosgene (0.37 g, 1.0 mmol, 2.0 eq.) at RT. The resulting mixture was stirred for 16 h at RT and 3 h at 60° C. Following this, concentrated under vacuum to get the solid residue which was purified by was purified normal phase silica-gel column to get the title compound as brown color semi solid (0.6 g 47%). LCMS: 236.9 [M+1]⁺; ¹H NMR (400 MHz, DMSO-d₆) δ ppm 8.94 (d, J=8.77 Hz, 1H) 3.77-3.88 (m, 1H) 3.33-3.44 (m, 3H) 1.56-1.69 (m, 1H) 1.32-1.46 (m, 1H) 0.85 (t, J=7.45 Hz, 3H).

### Step-3: Synthesis of (S)-3-(4-((S)-1-(6-chloro-2-oxo-1,2-dihydro-1,8-naphthyridin-3-yl)ethylamino)-6-methyl-1,3,5-triazin-2-yl)-4-ethyloxazolidin-2-one

In a microwave vial charged with (S)-1-(4-phenoxyphenyl)ethanamine hydrochloride (0.10 g, 0.46 mmol, 1.0 eq.), (S)-3-(4,6-dichloro-1,3,5-triazin-2-yl)-4-ethyloxazolidin-2-one (0.13 g, 0.51 mmol, 1.1 eq.) and N, N-Diisopropylethylamine (0.15 mL, 0.93 mmol, 2.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.012 g 8%), UPLC-MS (Method 7): Rt 4.31, m/z 440.4 [M+1]⁺; ¹H NMR (400 MHz, DMSO-d₆) δ ppm 9.16 (d, J=7.89 Hz, 1H) 9.05 (d, J=8.33 Hz, 1H) 7.30-7.42 (m, 5H) 7.08-7.18 (m, 2H) 6.91-7.02 (m, 5H) 5.05-5.17 (m, 2H) 4.50 (t, J=7.89 Hz, 2H) 4.40 (q, J=8.48 Hz, 2H) 4.14-4.21 (m, 2H) 1.73-1.81 (m, 1H) 1.41-1.52 (m, 5H) 1.14-1.27 (m, 1H) 0.84 (t, J=7.24 Hz, 1H) 0.73 (t, J=7.45 Hz, 3H).

### Example-37: Synthesis of (S)-3-(4-((S)-1-(6-chloro-2-oxo-1,2-dihydro-1,8-naphthyridin-3-yl)ethylamino)-6-methyl-1,3,5-triazin-2-yl)-4-ethyloxazolidin-2-one (Compound 1.53)

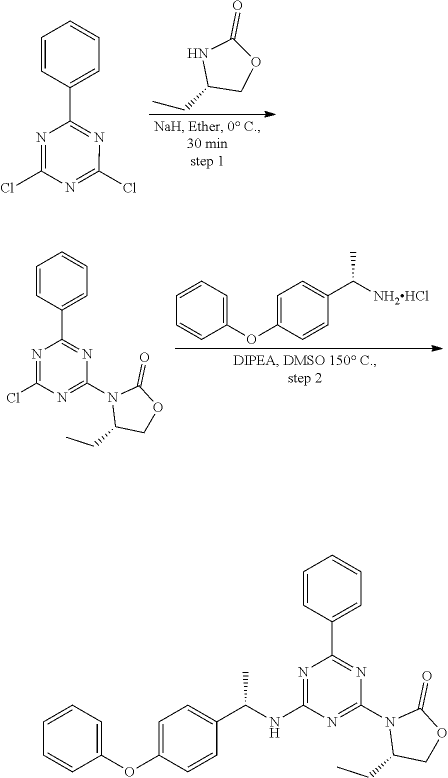

### Step-1: Synthesis of (S)-3-(4-chloro-6-phenyl-1,3,5-triazin-2-yl)-4-ethyloxazolidin-2-one

To a stirred solution of Sodium hydride (0.13 g, 3.3 mmol, 1.5 eq.) in Ether (20 mL) was added (S)-4-ethyloxazolidin-2-one (0.28 g, 2.4 mmol, 1.1 eq.) in Ether (3 mL) at 0° C. The resulting solution stirred for 15 min, following this 2,4-dichloro-6-phenyl-1,3,5-triazine (0.5 g, 2.2 mmol, 1.0 eq.) in Ether (2 mL) was added. The resulting mixture was stirred for another 30 min at same temperature. Following this, the reaction mixture diluted with saturated NH₄Cl (10 mL) and extracted with ethyl acetate (3×15 mL). The combined organic layers were washed with brine (30 mL), dried over anhydrous Na₂SO₄, filtered and concentrated under vacuum to get the solid residue which was purified by was purified normal phase silica-gel column chromatography provided title compound as off white solid (0.1 g, 21.1%). LCMS: 305.2 [M+1]⁺; ¹H NMR (400 MHz, DMSO-d₆) δ ppm 8.40 (d, J=7.89 Hz, 2H) 7.72 (t, J=7.24 Hz, 1H) 7.62 (t, J=7.67 Hz, 2H) 4.76 (br. s., 1H) 4.52 (t, J=8.33 Hz, 1H) 4.32 (dd, J=8.33, 2.63 Hz, 1H) 1.85-1.93 (m, 2H) 0.92 (t, J=7.24 Hz, 3H).

### Step-2: Synthesis of (S)-3-(4-((S)-1-(6-chloro-2-oxo-1,2-dihydro-1,8-naphthyridin-3-yl)ethylamino)-6-methyl-1,3,5-triazin-2-yl)-4-ethyloxazolidin-2-one

In a microwave vial charged with (S)-1-(4-phenoxyphenyl)ethanamine hydrochloride (0.10 g, 0.46 mmol, 1.0 eq.), (S)-3-(4-chloro-6-phenyl-1,3,5-triazin-2-yl)-4-ethyloxazolidin-2-one (0.14 g, 0.46 mmol, 1.0 eq.) and N, N-Diisopropylethylamine (0.12 mL, 0.93 mmol, 2.0 eq.), in DMSO (5 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.018 g 10%), UPLC-MS (Method 7): Rt 4.83, m/z 482.5 [M+1]⁺; ¹H NMR (400 MHz, DMSO-d₆) δ ppm 8.71 (d, J=7.89 Hz, 1H) 8.30-8.37 (m, 2H) 7.46-7.63 (m, 4H) 7.30-7.44 (m, 3H) 7.11 (t, J=7.45 Hz, 1H) 6.92-7.01 (m, 3H) 5.34-5.42 (m, 1H) 5.14-5.23 (m, 1H) 4.58-4.74 (m, 1H) 4.44 (dt, J=12.39, 8.50 Hz, 1H) 4.15-4.23 (m, 1H) 1.80-1.92 (m, 1H) 1.46-1.68 (m, 4H) 0.89 (t, J=7.45 Hz, 1H) 0.78 (t, J=7.24 Hz, 2H).

### Example-38: Synthesis of (S)-4-ethyl-3-(4-methyl-6-((S)-1-(4-methyl-2′-(trifluoromethyl)-3,4′-bipyridin-6-yl)ethylamino)-1,3,5-triazin-2-yl)oxazolidin-2-one (Compound 1.54)

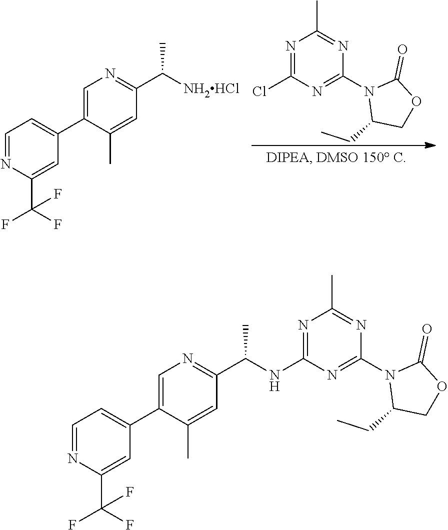

In a microwave vial charged with (S)-1-(4-methyl-2′-(trifluoromethyl)-3,4′-bipyridin-6-yl)ethanamine hydrochloride (0.15 g, 0.53 mmol, 1.0 eq.), (S)-3-(4-chloro-6-methyl-1,3,5-triazin-2-yl)-4-ethyloxazolidin-2-one (0.14 g, 0.58 mmol, 1.1 eq.) and N, N-Diisopropylethylamine (0.14 mL, 1.06 mmol, 2.0 eq.), in DMSO (5 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound (0.018 g 11%), UPLC-MS (Method 2): Rt 2.65, m/z 488.5 [M+1]⁺; ¹H NMR (400 MHz, DMSO-d₆) δ ppm 8.85 (d, J=4.82 Hz, 1H) 8.54 (d, J=7.02 Hz, 1H) 8.33-8.48 (m, 2H) 7.95 (s, 1H) 7.75-7.86 (m, 1H) 7.36-7.45 (m, 1H) 5.30-5.39 (m, 1H) 5.10-5.19 (m, 1H) 4.31-4.50 (m, 2H) 4.06-4.20 (m, 1H) 2.22-2.32 (m, 5H) 1.73-1.86 (m, 1H) 1.50 (d, J=7.02 Hz, 3H) 1.31-1.45 (m, 2H) 0.85 (t, J=7.24 Hz, 1H) 0.65 (t, J=7.24 Hz, 2H).

### Example-39: Synthesis of (S)-4-ethyl-3-(4-((S)-1-(6-methoxy-2-oxo-1,2-dihydroquinolin-3-yl)ethylamino)-6-methyl-1,3,5-triazin-2-yl)oxazolidin-2-one (Compound 1.55) and (S)-4-ethyl-3-(4-((R)-1-(6-methoxy-2-oxo-1,2-dihydroquinolin-3-yl)ethylamino)-6-methyl-1,3,5-triazin-2-yl)oxazolidin-2-one (Compound 1.56)

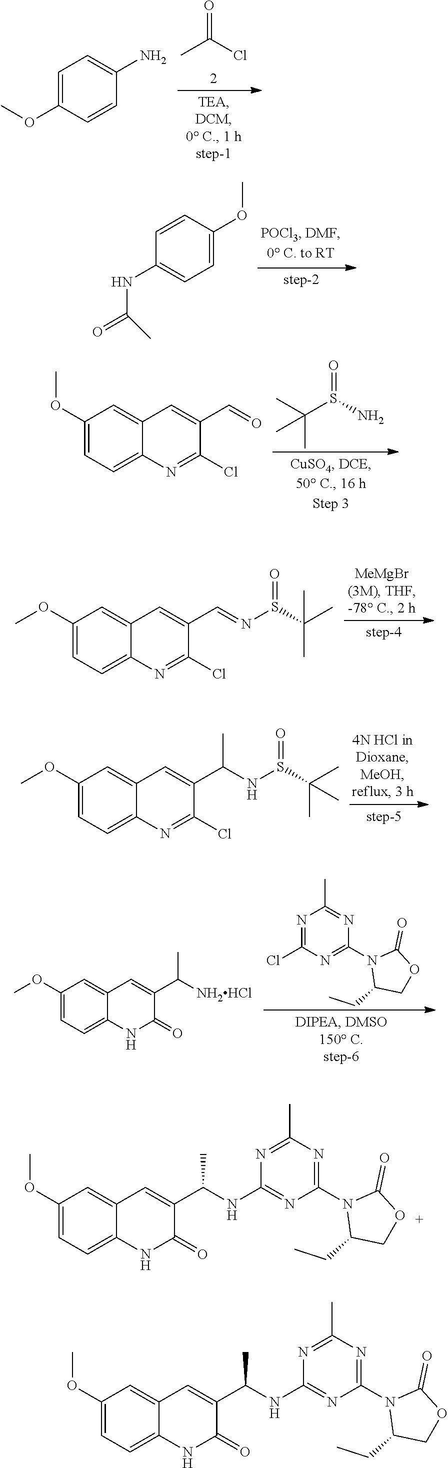

### Step-1: Synthesis of N-(4-methoxyphenyl)acetamide

To a stirred solution of 4-methoxyaniline (1.23 g, 10.0 mmol, 1.0 eq.) in DCM (20 mL) was added triethyl amine (4.15 mL, 30.0 mmol, 3.0 eq.). The resulting mixture was cooled to 0° C. and added acetyl chloride (1.17 mL, 15.0 mmol, 1.5 eq.) drop wise. The resulting mixture stirred for 1 h at same temperature. Following this, reaction mixture diluted with water (200 mL). The aqueous layer was separated extracted with DCM (3×30 mL). The combined organic layers were washed with brine (50 mL), dried over anhydrous Na₂SO₄, filtered and concentrated under vacuum to get title compound (1.3 g, 78%). LCMS: 166.1 [M+1]⁺; ¹H NMR (400 MHz, CHLOROFORM-d) δ ppm 7.35-7.43 (m, 2H) 7.26 (s, 1H) 7.09 (br. s., 1H) 6.83-6.91 (m, 2H) 3.79 (s, 3H) 2.15 (s, 3H).

### Step-2: Synthesis of 2-chloro-6-methoxyquinoline-3-carbaldehyde

DMF (1.51 mL, 25.0 mmol, 2.5 eq.) was cooled to 0° C. and added POCl₃ (6.5 mL, 70.0 mmol, 7.0 eq.) drop wise over 5 min. To the above solution added N-(4-methoxyphenyl)acetamide (1.66 g, 10.0 mmol, 1.0 eq.) dissolved in DMF (2 mL). The resulting mixture heated at 100° C. for 16 h. Following this, reaction mixture diluted with ice cold water (100 mL) and stirred for 1 h. Filtered the solid and under vacuum to get title compound as half white solid (1.0 g, 45%). ¹H NMR (400 MHz, DMSO-d₆) δ ppm 10.37 (s, 1H) 8.85 (s, 1H) 7.96 (d, J=9.21 Hz, 1H) 7.70 (d, J=2.63 Hz, 1H) 7.62 (dd, J=9.21, 2.63 Hz, 1H) 3.89-3.95 (m, 3H).

### Step-3: Synthesis of (S,E)-N-((2-chloro-6-methoxyquinolin-3-yl)methylene)-2-methylpropane-2-sulfinamide

To a stirred solution of 2-chloro-6-methoxyquinoline-3-carbaldehyde (1.0 g, 4.50 mmol, 1.0 eq.) and Copper (II) sulfate (1.43 g, 9.00 mmol, 2.0 eq.) in dichloroethane (20 mL) was added (S)-2-methylpropane-2-sulfinamide (2.18 g, 18.00 mmol, 4.0 eq.) at RT. The resulting mixture was heated at 50° C. for 16 h. Following this, reaction was allowed to cool to room temperature, filtered through celite pad, the celite pad washed with dichloromethane (30 mL). The combined filtrate dried over anhydrous Na₂SO₄ and concentrated under vacuum to get the solid residue which was purified by normal phase silica-gel column chromatography to get the title compound (1.25 g 85.7%). LCMS: 325.0 [M+1]⁺. ¹H NMR (400 MHz, DMSO-d₆) δ ppm 8.99 (s, 1H) 8.88 (s, 1H) 7.92 (d, J=9.21 Hz, 1H) 7.69 (d, J=2.63 Hz, 1H) 7.56 (dd, J=9.21, 3.07 Hz, 1H) 3.92 (s, 3H) 1.24-1.28 (m, 9H).

### Step-4: Synthesis of (S)—N-(1-(2-chloro-6-methoxyquinolin-3-yl)ethyl)-2-methylpropane-2-sulfinamide

To a stirred solution of (S,E)-N-((2-chloro-6-methoxyquinolin-3-yl)methylene)-2-methylpropane-2-sulfinamide (1.2 g, 3.70 mmol, 1.0 eq.) in Ether (25 mL) was added drop wise 3 M methylmagnesium bromide (6.8 mL, 18.50 mmol, 5.0 eq.) at −78° C. The resulting mixture was stirred for 2 h at same temperature and allowed to RT stirred for 1 h. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.65 g 51.6%). LCMS: 341.1 [M+1]⁺.

### Step-5: Synthesis of 3-(1-aminoethyl)-6-methoxyquinolin-2(1H)-one hydrochloride

To a stirred solution of (S)—N-(1-(2-chloro-6-methoxyquinolin-3-yl)ethyl)-2-methylpropane-2-sulfinamide (0.25 g, 0.73 mmol, 1.0 eq.) in MeOH (2 mL) was added 4N HCl in dioxane (2.0 mL, 7.3 mmol, 10.0 eq.) at RT. The resulting mixture was heated to reflux for 3 h. Following this, the reaction mixture was evaporated under reduced pressure to get title compound which is used to next step without further purification (0.11 g 59.2%). LCMS: 255.1 [M+1]⁺.

### Step-6: Synthesis of (S)-4-ethyl-3-(4-((S)-1-(6-methoxy-2-oxo-1,2-dihydroquinolin-3-yl)ethylamino)-6-methyl-1,3,5-triazin-2-yl)oxazolidin-2-one and (S)-4-ethyl-3-(4-((R)-1-(6-methoxy-2-oxo-1,2-dihydroquinolin-3-yl)ethylamino)-6-methyl-1,3,5-triazin-2-yl)oxazolidin-2-one

In a microwave vial charged with 3-(1-aminoethyl)-6-methoxyquinolin-2(1H)-one hydrochloride (0.1 g, 0.4 mmol, 1.0 eq.), (S)-3-(4-chloro-6-methyl-1,3,5-triazin-2-yl)-4-ethyloxazolidin-2-one (0.1 g, 0.40 mmol, 1.0 eq.) and N, N-Diisopropylethylamine (0.13 mL, 1.24 mmol, 3.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compounds as white solid. Compound 1.55 (0.02 g, 18%), UPLC-MS (Method 4): Rt 2.09, m/z 425.5 [M+1]⁺; ¹H NMR (400 MHz, DMSO-d₆) δ ppm 8.44 (d, J=7.02 Hz, 1H) 7.60 (s, 1H) 7.18-7.27 (m, 1H) 7.05-7.18 (m, 3H) 5.09 (t, J=6.80 Hz, 1H) 4.30-4.44 (m, 2H) 4.13 (br. s., 3H) 4.04 (d, J=6.14 Hz, 1H) 3.73-3.77 (m, 4H) 3.16 (s, 8H) 2.28 (s, 3H) 2.22 (s, 1H) 2.06-2.10 (m, 2H) 1.40 (d, J=7.02 Hz, 4H) 1.21-1.27 (m, 1H) 0.84 (t, J=7.45 Hz, 1H) 0.52 (t, J=7.45 Hz, 3H). Compound 1.56 (0.018 g, 17%), UPLC-MS (Method 4): Rt 2.15, m/z 425.5 [M+1]⁺; ¹H NMR (400 MHz, DMSO-d₆) δ ppm 8.41 (d, J=7.45 Hz, 1H) 7.70-7.77 (m, 1H) 7.06-7.27 (m, 4H) 5.20 (t, J=7.02 Hz, 1H) 4.24-4.44 (m, 2H) 4.06-4.13 (m, 1H) 3.73-3.78 (m, 3H) 2.21-2.29 (m, 3H) 1.77-1.89 (m, 1H) 1.64-1.77 (m, 1H) 1.34-1.45 (m, 4H) 1.24 (d, J=6.58 Hz, 1H) 0.79-0.90 (m, 4H).

### Example-40: Synthesis of (S)-3-(4-((6-chloro-2-oxo-1,2-dihydroquinolin-3-yl)methylamino)-6-methyl-1,3,5-triazin-2-yl)-4-ethyloxazolidin-2-one (Compound 1.57)

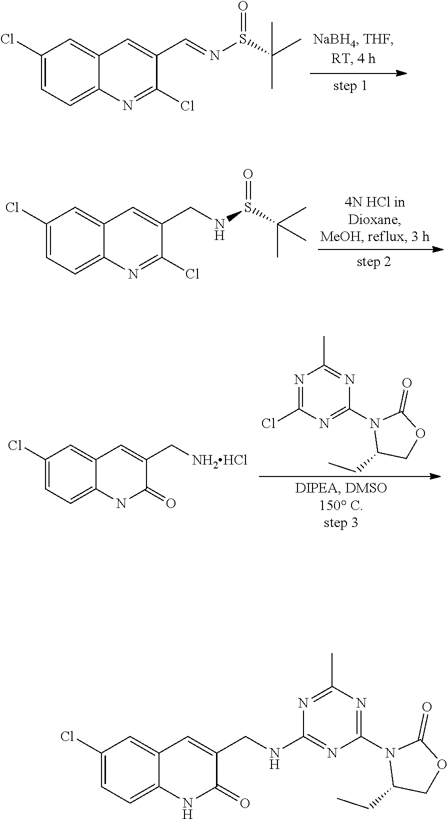

### Step-1: Synthesis of (S)—N-((2,6-dichloroquinolin-3-yl)methyl)-2-methylpropane-2-sulfinamide

To a stirred solution of (S,E)-N-((2,6-dichloroquinolin-3-yl)methylene)-2-methylpropane-2-sulfinamide (0.3 g, 0.91 mmol, 1.0 eq.) in THF (25 mL) was added Sodiumborohydride (0.13 g, 3.63 mmol, 4.0 eq.) at RT. The resulting mixture was stirred for 4 h at same temperature. The reaction was then quenched by careful addition of MeOH (5 mL) and concentrated to give crude solid residue which was purified by normal phase silica-gel column chromatography to get the title compound (0.3 g, 99%). LCMS: 331.3 [M+1]⁺.

### Step-2: Synthesis of 3-(aminomethyl)-6-chloroquinolin-2(1H)-one hydrochloride

To a stirred solution of (S)—N-((2,6-dichloroquinolin-3-yl)methyl)-2-methylpropane-2-sulfinamide (0.3 g, 0.95 mmol, 1.0 eq.) in MeOH (2 mL) was added 4N HCl in dioxane (2.0 mL, 7.3 mmol, 10.0 eq.) at RT. The resulting mixture was heated to reflux for 3 h. Following this, the reaction mixture was evaporated under reduced pressure to get title compound which is used to next step without further purification (0.2 g 99.2%). LCMS: 255.1 [M+1]⁺.

### Step-3: Synthesis of (S)-3-(4-((6-chloro-2-oxo-1,2-dihydroquinolin-3-yl)methylamino)-6-methyl-1,3,5-triazin-2-yl)-4-ethyloxazolidin-2-one

In a microwave vial charged with 3-(aminomethyl)-6-chloroquinolin-2(1H)-one hydrochloride (0.10 g, 0.48 mmol, 1.0 eq.), (S)-3-(4-chloro-6-methyl-1,3,5-triazin-2-yl)-4-ethyloxazolidin-2-one (0.11 g, 0.48 mmol, 1.1 eq.) and N, N-Diisopropylethylamine (0.12 mL, 1.06 mmol, 2.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound (0.025 g 12%), UPLC-MS (Method 4): Rt 2.25, m/z 415.4 [M+1]⁺; ¹H NMR (400 MHz, DMSO-d₆) δ ppm 12.00 (s, 1H) 8.30-8.36 (m, 1H) 8.20 (br. s., 1H) 7.84 (s, 1H) 7.78 (s, 1H) 7.66-7.73 (m, 1H) 7.48 (dd, J=8.55, 2.41 Hz, 1H) 7.28-7.34 (m, 1H) 4.29-4.47 (m, 5H) 4.18 (d, J=5.70 Hz, 1H) 4.07 (d, J=8.33 Hz, 2H) 2.23-2.31 (m, 4H) 1.82 (d, J=8.33 Hz, 2H) 1.60 (br. s., 1H) 1.46-1.57 (m, 2H) 0.86 (t, J=7.24 Hz, 2H) 0.60 (t, J=7.24 Hz, 3H).

### Example-41: Synthesis of (S)-4-ethyl-3-(4-((S)-1-(4-(4-fluorophenoxy)phenyl)ethylamino)-6-methyl-1,3,5-triazin-2-yl)oxazolidin-2-one (Compound 1.58)

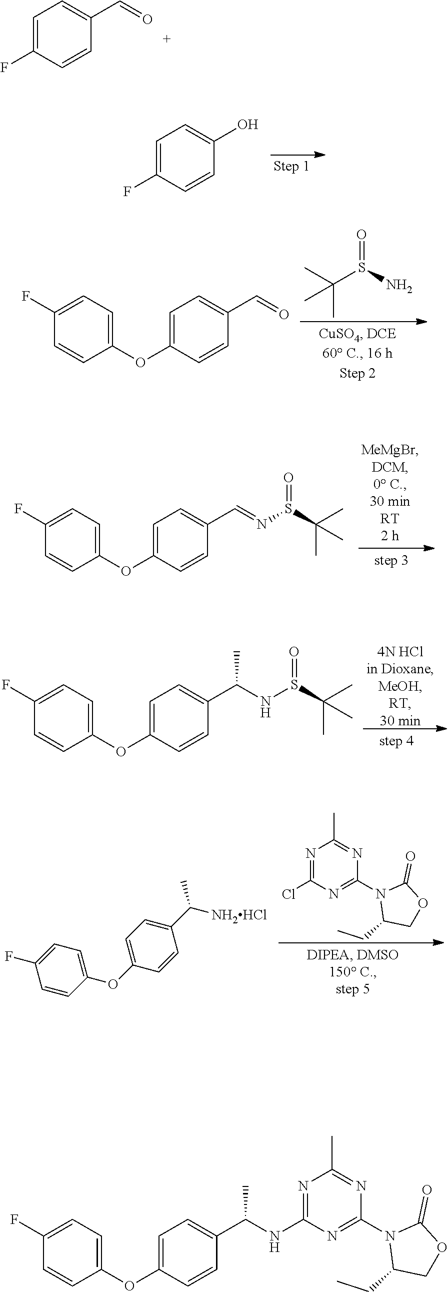

### Step-1: Synthesis of 4-(4-fluorophenoxy)benzaldehyde

To a stirred solution of 4-fluorobenzaldehyde (2 g, 16.0 mmol, 1.0 eq.) and 4-fluorophenol (1.98 g, 17.7 mmol, 1.1 eq.) in DMF (20 mL) was added K₂CO₃ (6.62 g, 48.0 mmol, 3 eq.) The resulting mixture heated at 110° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound as white solid (3.0 g, 86%). LCMS: 216.9 [M+1]⁺; ¹H NMR (400 MHz, CHLOROFORM-d) δ ppm 9.92 (s, 1H) 7.80-7.89 (m, 2H) 6.98-7.16 (m, 7H).

### Step-2: Synthesis of (R,E)-N-(4-(4-fluorophenoxy)benzylidene)-2-methylpropane-2-sulfinamide

To a stirred solution of 4-(4-fluorophenoxy)benzaldehyde (3.0 g, 13.0 mmol, 1.0 eq.) and Copper(II) sulfate (4.4 g, 27.0 mmol, 2.0 eq.) in dichloroethane (40 mL) was added (R)-2-methylpropane-2-sulfinamide (2.37 g, 19.0 mmol, 1.0 eq.) at RT. The resulting mixture was heated at 60° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound as semi solid (2 g, 48.2%). LCMS: 320.2 [M+1]⁺; ¹H NMR (400 MHz, DMSO-d₆) δ ppm 8.50 (s, 1H) 7.95 (m, J=8.77 Hz, 2H) 7.26-7.34 (m, 2H) 7.17-7.23 (m, 2H) 7.07 (m, J=8.33 Hz, 2H) 1.17 (s, 9H).

### Step-3: Synthesis of (R)-2-methyl-N—((S)-1-(4-phenoxyphenyl)ethyl)propane-2-sulfinamide

To a stirred solution of (R,E)-2-methyl-N-(4-phenoxybenzylidene)propane-2-sulfinamide (0.5 g, 1.56 mmol, 1.0 eq.) in DCM (10 mL) was added drop wise 3 molar methylmagnesium bromide (2.0 mL, 6.24 mmol, 4.0 eq.) at 0° C. The resulting mixture was stirred for 30 min at same temperature followed by 2 h at RT. The reaction was then quenched by careful addition of saturated NH₄Cl (20 mL). The aqueous layer was separated and extracted with DCM (3×30 mL). The combined organic layers were dried over Na₂SO₄, filtered and concentrated to give crude solid residue which was purified by normal phase silica-gel column chromatography to get the title compound (0.18 g 35%). LCMS: 336.1 [M+1]⁺.

### Step-4: Synthesis of (S)-1-(4-(4-fluorophenoxy)phenyl)ethanamine hydrochloride

To a stirred solution of (R)-2-methyl-N—((S)-1-(4-phenoxyphenyl)ethyl)propane-2-sulfinamide (0.18 g, 0.53 mmol, 1.0 eq.) in methanol (5 mL) was added 4N HCl in dioxane (0.66 mL, 2.65 mmol, 5.0 eq.) at RT. The resulting mixture was stirred for 30 min. Following this, the reaction mixture was evaporated under reduced pressure to get solid. This solid washed with ether and evaporated to give title compound (0.15 g crude). LCMS: 232.0 [M+1]⁺; ¹H NMR (400 MHz, DMSO-d₆) δ ppm 8.31 (br. s., 2 H) 7.50 (d, J=8.77 Hz, 2H) 7.23-7.29 (m, 2H) 7.01-7.11 (m, 4H) 4.38-4.44 (m, 1H) 1.49 (d, J=7.02 Hz, 3H)

### Step-5: Synthesis of (S)-4-isopropyl-3-(4-methyl-6-((S)-1-(4-phenoxyphenyl)ethylamino)-1,3,5-triazin-2-yl)oxazolidin-2-one

In a microwave vial charged with (S)-1-(4-(4-fluorophenoxy)phenyl)ethanamine hydrochloride (0.15 g, 0.64 mmol, 1.0 eq.) and (S)-3-(4-chloro-6-methyl-1,3,5-triazin-2-yl)-4-ethyloxazolidin-2-one (0.24 g, 1.06 mmol, 1.5 eq.) in DMSO (3 mL) was added N, N-Diisopropylethylamine (0.25 g, 1.9 mmol, 3.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound (0.030 g, 6%), UPLC-MS (Method 4): Rt 2.61, m/z 438.4 [M+1]⁺; ¹H NMR (400 MHz, DMSO-d₆) δ ppm 8.54 (d, J=7.89 Hz, 1H) 7.33-7.41 (m, 3H) 7.16-7.24 (m, 2H) 6.89-7.07 (m, 5H) 5.04-5.12 (m, 1H) 4.47-4.60 (m, 2H) 4.33-4.43 (m, 2H) 4.09-4.17 (m, 1H) 2.24 (s, 3H) 1.40-1.46 (m, 4H) 0.77-0.86 (m, 2H) 0.72 (t, J=7.45 Hz, 3H).

### Example-42: Synthesis of (R)-3-(4-((S)-1-(6-chloro-2-oxo-1,2-dihydroquinolin-3-yl)ethylamino)-6-methyl-1,3,5-triazin-2-yl)-4-((S)-1-fluoroethyl) oxazolidin-2-one (Compound 1.59) and (R)-3-(4-((R)-1-(6-chloro-2-oxo-1,2-dihydroquinolin-3-yl)ethylamino)-6-methyl-1,3,5-triazin-2-yl)-4-((S)-1-fluoroethyl)oxazolidin-2-one (Compound 1.60)

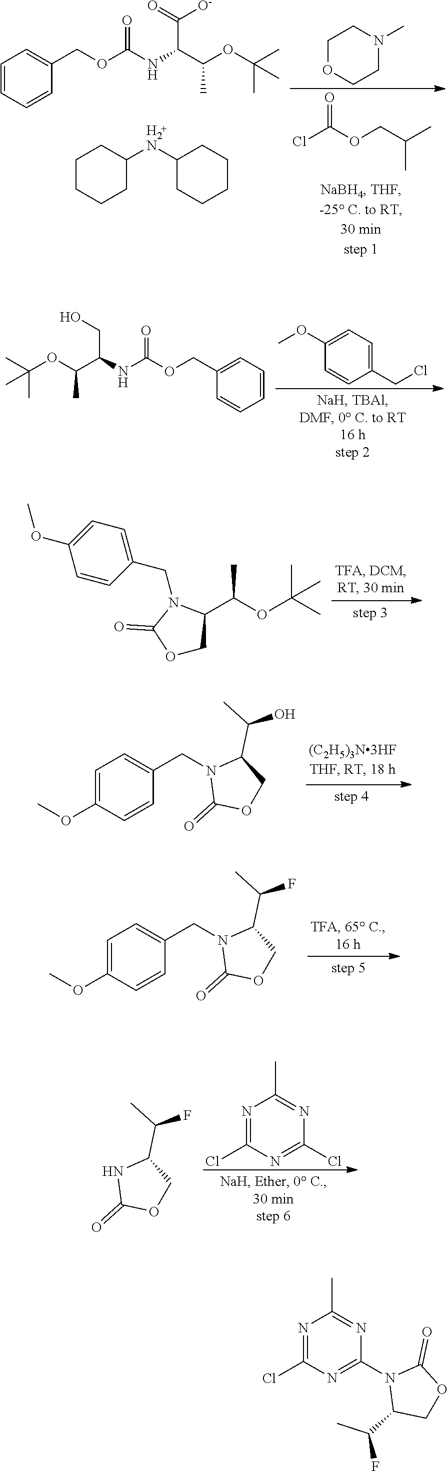

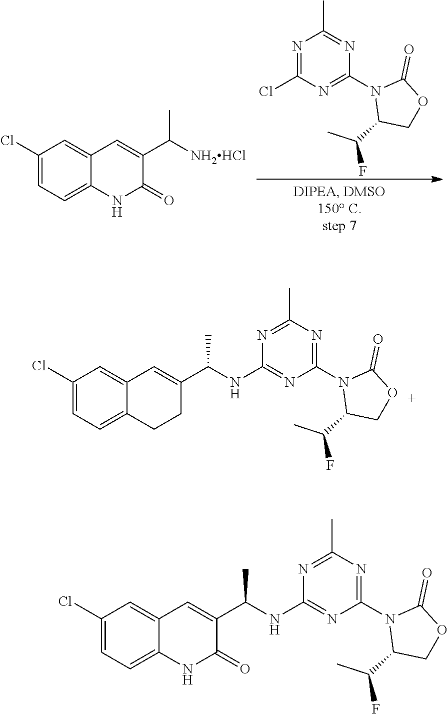

### Step-1: Synthesis of benzyl (2R,3R)-3-tert-butoxy-1-hydroxybutan-2-ylcarbamate

A solution of (2S,3R)-2-(((benzyloxy)carbonyl)amino)-3-(tert-butoxy)butanoic acid dicyclohexylammonium salt (1.0 g, 3.23 mmol, 1.0 eq.) in 20 ml of THF and isobutyl chloroformate (0.5 mL, 3.87 mmol, 1.2 eq.) at −25° C. was added N-methylmorpholine (0.425 g, 3.87 mmol, 1.2 eq.), the mixture was stirred at same temperature for 10 min and filtered. The filtrate was cooled to −20° C. and to it was added NaBH₄ (0.18 g, 4.89 mmol, 1.5 eq.) followed by 4 ml of water immediately afterwards. The reaction mixture was stirred at same temperature for 5 min. then gradually warmed to room temperature for 25 min, poured into water (20 mL) and extracted with ethyl acetate (2×40 mL). The combined organic phases were washed with water, brine and dried over Na₂SO₄. The solvent was removed to yield title compound as clear oil (1.0 g crude). ¹H NMR (400 MHz, CHLOROFORM-d) δ ppm 7.29-7.44 (m, 5H) 5.23 (d, J=11.84 Hz, 1H) 5.11-5.15 (m, 1H) 4.13-4.18 (m, 1H) 3.74 (t, J=3.95 Hz, 1H) 3.58-3.70 (m, 2H) 1.16-1.21 (s, 9H) 0.89-0.94 (m, 3H).

### Step-2: Synthesis of (R)-4-((R)-1-tert-butoxyethyl)-3-(4-methoxybenzyl)oxazolidin-2-one

To a solution of benzyl ((2R,3R)-3-(tert-butoxy)-1-hydroxybutan-2-yl)carbamate (1.0 g, 3.3 mmol, 1 eq.) in 20 mL DMF was added NaH (0.27 g, 6.77 mmol, 2.0 eq.) at 0° C. The reaction mixture was stirred for 30 min at 0° C. To the reaction mixture were added 4-methoxybenzyl chloride (0.69 mL, 5.07 mmol, 1.5 eq.) and tetrabutylammonium iodide (0.12 g, 0.33 mmol, 0.1 eq.) and the resulting mixture was warmed to room temperature and stirred for 15.5 h. The reaction mixture was poured into ice water (50 mL) forming a white suspension. EtOAc (100 mL) was added and the resulting mixture was stirred for 5 min to form a clear two layer solution. After separation, the aqueous phase was extracted with EtOAc (3×100 mL). The combined organic solution was washed with brine (80 mL), dried over Na₂SO₄, filtered and concentrated to get crude. The crude purified by normal phase silica-gel column to get title compound (0.36 g 34%). ¹H NMR (400 MHz, CDCl3) δ 4.33 (t, J=8.7 Hz, 1H), 4.07 (dd, J=8.9, 5.5 Hz, 1H), 3.67-3.58 (m, 1H), 3.58-3.49 (m, 1H), 1.13 (s, 9H), 1.02 (d, J=6.0 Hz, 3H).

### Step-3: Synthesis of (R)-4-((R)-1-hydroxyethyl)-3-(4-methoxybenzyl)oxazolidin-2-one

A solution of (R)-4-((R)-1-(tert-butoxy)ethyl)-3-(4-methoxybenzyl)oxazolidin-2-one (0.3 g, 0.97 mmol, 1.0 eq.) in DCM (5 mL) was treated with TFA (2 mL) at room temperature for 20 min. The reaction mixture was concentrated in vacuo, then diluted with DCM (10 mL), and again concentrated. This procedure was repeated three times to get title compound isolated as (0.39 g crude). LCMS: 250.0 [M+1]⁺

### Step-4: Synthesis of (S)-4-((R)-1-fluoroethyl)-3-(4-methoxybenzyl)oxazolidin-2-one

To a cooled (0° C.) solution of (R)-4-((R)-1-hydroxyethyl)-3-(4-methoxybenzyl)oxazolidin-2-one (0.22 g, 0.9 mmol, 1.0 eq.) in 10 mL MeCN were added triethylamine (1.14 mL, 8.1 mmol, 9.0 eq.) followed by perfluoro-1-butanesulfonyl fluoride (0.49 mL, 2.7 mmol, 3.0 eq.) and NEt3(HF)3 (0.45 mL, 2.8 mmol, 3.1 eq.) and the resulting mixture was stirred at 0° C. for 70 min. The reaction mixture was diluted with water (6 mL) and extracted with EtOAc (3×10 mL). Combined organics were washed with water (30 mL), brine (30 mL), dried over Na₂SO₄, filtered and concentrated. The crude purified by normal phase silica-gel column chromatography to get title compound (0.22 g 96%). LCMS: 253.9 [M+1]⁺

### Step-5: Synthesis of (S)-4-((R)-1-fluoroethyl)oxazolidin-2-one

A solution of (S)-4-((R)-1-fluoroethyl)-3-(4-methoxybenzyl)oxazolidin-2-one (1.2 g 4.8 mmol, 1.0 eq.) in 22 mL TFA was heated at 65° C. for 16 h. The reaction mixture was concentrated to remove TFA. The crude mixture was purified by normal phase silica-gel column chromatography (EtOAc/CH₂Cl₂, 0 to 100%) gave title compound as brown oil (0.52 g 82%). ¹H NMR (400 MHz, CHLOROFORM-d) δ ppm 5.86 (br. s., 1H) 4.67 (quin, J=6.03 Hz, 1H) 4.48-4.61 (m, 2H) 4.32 (dd, J=9.21, 4.82 Hz, 1H) 3.90-4.00 (m, 1H) 1.33-1.43 (m, 3H)

### Step-6: Synthesis of (R)-3-(4-chloro-6-methyl-1,3,5-triazin-2-yl)-4-((S)-1-fluoroethyl)oxazolidin-2-one

To a stirred solution of Sodium hydride (0.07 g, 1.8 mmol, 1.5 eq.) in Ether (10 mL) was added (S)-4-((R)-1-fluoroethyl)oxazolidin-2-one (0.2 g, 1.2 mmol, 0.9 eq.) in Ether (2 mL) at 0° C. The resulting solution stirred for 15 min, following this 2,4-dichloro-6-methyl-1,3,5-triazine (0.19 g, 1.4 mmol, 1.1 eq.) in ether (2 mL) was added. The resulting mixture was stirred for another 30 min at same temperature. The reaction mixture concentrated under vacuum to get the solid residue which was purified by was purified normal phase silica-gel column chromatography provided title compound (0.13 g, 41.2%). LCMS: 260.9 [M+1]⁺; ¹H NMR (400 MHz, DMSO-d₆) δ ppm 5.30 (d, J=5.70 Hz, 1H) 5.18 (d, J=7.02 Hz, 1H) 4.82 (d, J=7.02 Hz, 1H) 4.75 (d, J=7.45 Hz, 1H) 4.56 (dd, J=9.21, 2.63 Hz, 1H) 4.47 (t, J=8.99 Hz, 1H) 2.54 (s, 3H) 1.31-1.42 (m, 3H).

### Step-7: Synthesis of (R)-3-(4-((S)-1-(6-chloro-2-oxo-1,2-dihydroquinolin-3-yl)ethylamino)-6-methyl-1,3,5-triazin-2-yl)-4-((S)-1-fluoroethyl)oxazolidin-2-one and (R)-3-(4-((R)-1-(6-chloro-2-oxo-1,2-dihydroquinolin-3-yl)ethylamino)-6-methyl-1,3,5-triazin-2-yl)-4-((S)-1-fluoroethyl)oxazolidin-2-one

In a microwave vial charged with 3-(1-aminoethyl)-6-chloroquinolin-2(1H)-one hydrochloride (0.07 g, 0.31 mmol, 1.0 eq.), (R)-3-(4-chloro-6-methyl-1,3,5-triazin-2-yl)-4-((S)-1-fluoroethyl)oxazolidin-2-one (0.08 g, 0.31 mmol, 1.0 eq.) and N, N-Diisopropylethylamine (0.16 mL, 0.94 mmol, 3.0 eq.), in DMSO (2 mL). The resulting mixture was heated at 150° C. for 90 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compounds as white solid Compound 1.59 (0.016 g, 12%), UPLC-MS (Method 4): Rt 2.30, m/z 447.4 [M+1]⁺; ¹H NMR (400 MHz, DMSO-d₆) δ ppm 8.54 (d, J=7.02 Hz, 1H) 7.78 (s, 1H) 7.66 (s, 1H) 7.50 (d, J=8.33 Hz, 1H) 7.32 (d, J=8.77 Hz, 1H) 5.32 (d, J=5.26 Hz, 1H) 5.00-5.10 (m, 1H) 4.62-4.76 (m, 1H) 4.38-4.59 (m, 2H) 4.34 (d, J=5.70 Hz, 2H) 2.30 (s, 2H) 2.23 (s, 1H) 1.89 (s, 1H) 1.75 (s, 1H) 1.29-1.44 (m, 3H) 1.02-1.14 (m, 2H). Compound 1.60 (0.012 g, 10%), UPLC-MS (Method 4): Rt 2.35, m/z 447.4 [M+1]⁺; ¹H NMR (400 MHz, DMSO-d₆) δ ppm 8.54 (d, J=7.45 Hz, 1H) 7.71-7.80 (m, 2H) 7.45-7.54 (m, 1H) 7.27-7.35 (m, 1H) 5.19-5.44 (m, 2H) 5.09-5.19 (m, 1H) 4.28-4.50 (m, 3H) 2.28 (s, 2H) 2.22 (s, 1H) 1.35-1.47 (m, 4H) 1.32 (d, J=6.58 Hz, 2H).

### Example-43: Synthesis of (S)-4-ethyl-3-(4-methyl-6-((R)-1-(4-(pyridin-2-ylmethoxy)phenyl)ethylamino)-1,3,5-triazin-2-yl)oxazolidin-2-one (Compound 1.61) and (S)-4-ethyl-3-(4-methyl-6-((S)-1-(4-(pyridin-2-ylmethoxy)phenyl)ethylamino)-1,3,5-triazin-2-yl)oxazolidin-2-one (Compound 1.62)

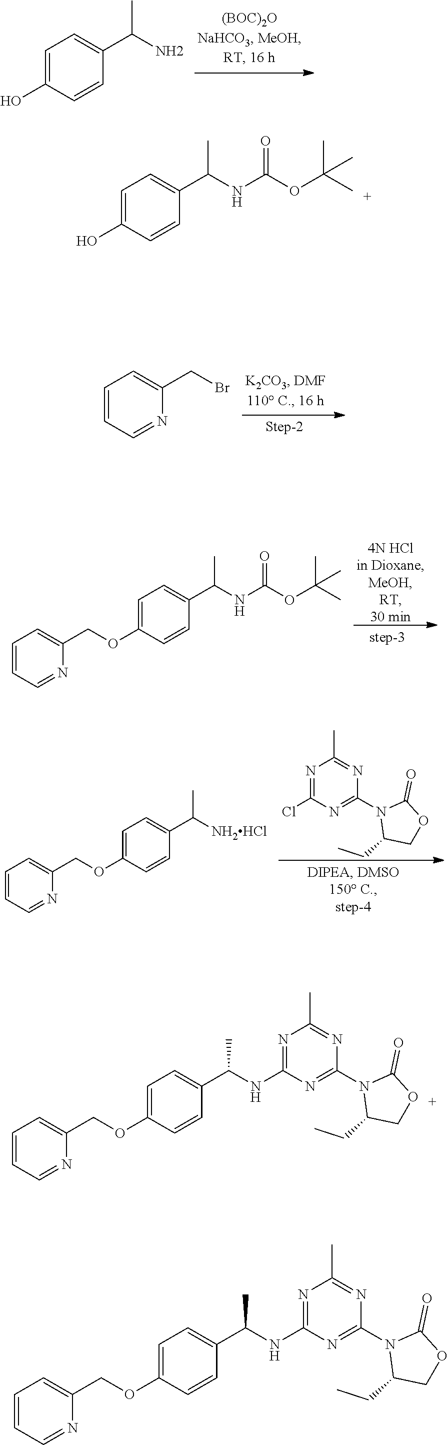

### Step-1: Synthesis of tert-butyl 1-(4-hydroxyphenyl)ethylcarbamate

To a stirred solution of 4-(1-aminoethyl)phenol (2.3 g, 16.0 mmol, 1 eq.) in MeOH (25 mL) was added NaHCO₃ (2.8 g, 33.0 mmol, 2 eq.) and di-tert-butyl dicarbonate (4.18 g, 19.2 mmol, 1.2 eq.) at RT. The resulting mixture stirred for 16 h. After completion of starting material, reaction mixture filtered and evaporated to give crude. The crude purified by normal phase silica-gel column to get title compound (1.5 g 39%).). %). ¹H NMR (400 MHz, DMSO-d₆) δ ppm 9.20 (s, 1H) 7.21 (d, J=7.89 Hz, 1H) 7.07 (m, J=8.33 Hz, 2H) 6.67 (m, J=8.77 Hz, 2H) 4.46-4.56 (m, 1H) 1.35 (s, 9H) 1.24 (d, J=7.02 Hz, 4H).

### Step-2: Synthesis of tert-butyl 1-(4-(pyridin-2-ylmethoxy)phenyl)ethylcarbamate

To a stirred solution of 2-(bromomethyl)pyridine (0.5 g, 2.9 mmol, 1.0 eq.) and tert-butyl 1-(4-hydroxyphenyl)ethylcarbamate (0.62 g, 2.6 mmol, 0.9 eq.) in DMF (10 mL) was added K₂CO₃ (1.2 g, 8.7 mmol, 3 eq.) at RT. The resulting mixture heated at 110° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound (0.5 g, 52.5%). LCMS: 329.1 [M+1]⁺; ¹H NMR (400 MHz, DMSO-d₆) δ ppm 8.57 (d, J=4.38 Hz, 1H) 7.82 (td, J=7.67, 1.75 Hz, 1H) 7.50 (d, J=7.89 Hz, 1H) 7.28-7.37 (m, 2H) 7.20 (m, J=8.77 Hz, 2H) 6.95 (m, J=8.77 Hz, 2H) 5.15 (s, 2H) 4.52-4.60 (m, 1H) 1.36 (s, 9H) 1.26 (d, J=7.02 Hz, 4H).

### Step-3: Synthesis of 1-(4-(pyridin-2-ylmethoxy)phenyl)ethanamine hydrochloride

To a stirred solution of Synthesis of tert-butyl 1-(4-(pyridin-2-ylmethoxy)phenyl)ethylcarbamate (0.4 g, 1.2 mmol, 1.0 eq.) in methanol (10 mL) was added 4N HCl in dioxane (0.1 mL, 3.6 mmol, 3.0 eq.) at RT. The resulting mixture was stirred for 30 min. Following this, the reaction mixture was evaporated under reduced pressure to get solid. This solid was washed with ether and evaporated to give title compound as white color solid (0.25 g 91.3%). LCMS: 229.0 [M+1]⁺; ¹H NMR (400 MHz, DMSO-d₆) δ ppm 8.75-8.82 (m, 1H) 8.52 (br. s., 2H) 8.23-8.34 (m, 1H) 7.82-7.93 (m, 1H) 7.69-7.79 (m, 1H) 7.48 (m, J=8.33 Hz, 2H) 7.24 (d, J=8.77 Hz, 1H) 7.10 (m, J=8.77 Hz, 2H) 7.00 (d, J=8.77 Hz, 1H) 5.35-5.43 (m, 2H) 4.26-4.40 (m, 1H) 1.49 (d, J=6.58 Hz, 3H) 1.35 (br. s., 2H) 1.26 (d, J=7.02 Hz, 1H).

### Step-4: Synthesis of (S)-4-ethyl-3-(4-methyl-6-((S)-1-(4-(pyridin-2-ylmethoxy)phenyl)ethylamino)-1,3,5-triazin-2-yl)oxazolidin-2-one and (S)-4-ethyl-3-(4-methyl-6-((R)-1-(4-(pyridin-2-ylmethoxy)phenyl)ethylamino)-1,3,5-triazin-2-yl)oxazolidin-2-one

In a microwave vial charged with 1-(4-(pyridin-2-ylmethoxy)phenyl)ethanamine hydrochloride (0.15 g, 0.65 mmol, 1.0 eq.), (S)-3-(4-chloro-6-methyl-1,3,5-triazin-2-yl)-4-ethyloxazolidin-2-one (0.17 g, 0.72 mmol, 1.1 eq.) and N, N-Diisopropylethylamine (0.2 mL, 1.31 mmol, 2.0 eq.), in DMSO (5 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compounds as white solid. Compound 161 (0.052 g, 19%), UPLC-MS (Method 2): Rt 2.25, m/z 435.4 [M+1]⁺; ¹H NMR (400 MHz, DMSO-d₆) δ ppm 8.56 (d, J=4.38 Hz, 1H) 8.45 (d, J=8.33 Hz, 1H) 7.82 (td, J=7.67, 1.75 Hz, 1H) 7.49 (d, J=7.89 Hz, 1H) 7.27-7.37 (m, 3H) 6.92-7.01 (m, 2H) 5.14 (s, 2H) 4.99-5.07 (m, 1H) 4.33-4.43 (m, 2H) 4.14 (d, J=6.58 Hz, 1H) 2.19-2.27 (m, 3H) 1.72-1.91 (m, 2H) 1.40 (d, J=7.02 Hz, 3H) 0.78-0.90 (m, 3H). Compound 1.62 (0.076 g, 21%), UPLC-MS (Method 2): Rt 2.18, m/z 435.4 [M+1]⁺; ¹H NMR (400 MHz, DMSO-d₆) δ ppm 8.56 (d, J=3.07 Hz, 1H) 8.48 (d, J=7.89 Hz, 1H) 8.32 (d, J=8.33 Hz, 1H) 7.81 (td, J=7.67, 1.75 Hz, 2H) 7.44-7.53 (m, 2H) 7.23-7.37 (m, 4H) 6.89-7.01 (m, 3H) 5.11-5.15 (m, 3H) 5.01-5.07 (m, 1H) 4.47-4.58 (m, 2H) 4.33-4.42 (m, 2H) 4.09-4.16 (m, 2H) 2.19-2.27 (m, 4H) 1.74-1.81 (m, 1H) 1.50-1.57 (m, 1H) 1.37-1.43 (m, 4H) 0.82 (t, J=7.45 Hz, 1H) 0.71 (t, J=7.24 Hz, 3H).

### Example-44: Synthesis of (S)-3-(4-(dimethylamino)-6-((S)-1-(4-phenoxyphenyl)ethylamino)-1,3,5-triazin-2-yl)-4-ethyloxazolidin-2-one (Compound 1.63)

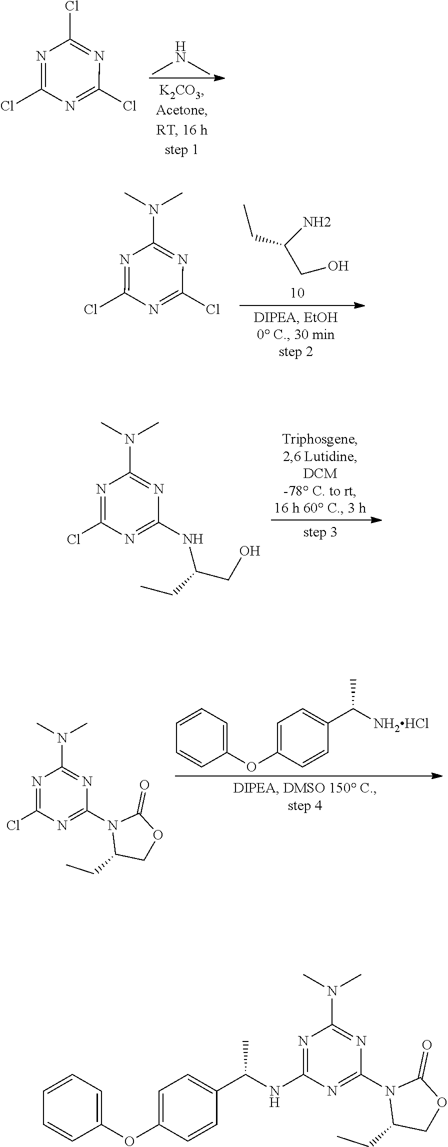

### Step-1: Synthesis of 4,6-dichloro-N,N-dimethyl-1,3,5-triazin-2-amine

To a stirred solution of 2,4,6-trichloro-1,3,5-triazine (1.0 g, 5.0 mmol, 1.0 eq.) in acetone (20 mL) was added K₂CO₃ (1.38 g, 10 mmol, 2.0 eq.) and dimethylamine (0.36 g, 8 mmol, 1.5 eq.) at RT. The reaction mixture stirred for 16 h. On completion of starting material, the reaction mixture filtered and evaporated to give title compound as white solid (0.3 g 31%). LCMS: 192.9 [M+1]⁺; ¹H NMR (400 MHz, CHLOROFORM-d) δ ppm 3.23 (s, 6H).

### Step-2: Synthesis of (S)-2-(4-chloro-6-(dimethylamino)-1,3,5-triazin-2-ylamino)butan-1-ol

To a stirred solution of 4,6-dichloro-N,N-dimethyl-1,3,5-triazin-2-amine (0.3 g, 1.5 mmol, 1.0 eq.), (S)-2-aminobutan-1-ol (0.13 g, 1.5 mmol, 1.0 eq.) in EtOH (10 mL) was added DIPEA (0.38 g, 3.0 mmol, 2.0 eq.) at 0° C. The resulting mixture was stirred for 30 min. Following this, concentrated under vacuum to get the solid residue which was purified by was purified normal phase silica-gel column to get the title compound as brown color semi solid (0.25 g 68%). LCMS: 246.1 [M+1]⁺; ¹H NMR (400 MHz, DMSO-d₆) δ ppm 7.51 (d, J=8.33 Hz, 1H) 4.58-4.68 (m, 1H) 3.75-3.86 (m, 1H) 3.35-3.45 (m, 1H) 2.94-3.13 (m, 5H) 1.55-1.67 (m, 1H) 1.37 (dd, J=14.25, 8.11 Hz, 1H) 1.15-1.29 (m, 1H) 0.84 (quin, J=6.69 Hz, 3H).

### Step-3: Synthesis of (S)-3-(4-chloro-6-(dimethylamino)-1,3,5-triazin-2-yl)-4-ethyloxazolidin-2-one

To a stirred solution of (S)-2-(4-chloro-6-(dimethylamino)-1,3,5-triazin-2-ylamino)butan-1-ol (0.25 g, 1.0 mmol, 1.0 eq.), 2,6 lutidine (0.52 mL, 4.5 mmol, 4.5 eq.) in DCM (10 mL) was added Triphosgene (0.14 g, 0.5 mmol, 0.5 eq.) at −78° C. The resulting mixture was stirred for 10 min at same temperature then allowed to RT and stirred for 16 h. Following this, the reaction mixture hated at 60° C. for 3 h. On completion of starting material, cool the reaction mixture quenched with saturated ammonium chloride solution (10 mL) and extracted with DCM (3×20 mL). Combined organics were washed with water (40 mL), brine (40 mL), dried over Na₂SO₄, filtered and concentrated under vacuum to get the solid residue which was purified by was purified normal phase silica-gel column to get the title compound as off white solid (0.2 g 73%). LCMS: 272.0 [M+1]⁺; ¹H NMR (400 MHz, DMSO-d₆) δ ppm 4.53-4.60 (m, 1H) 4.43 (t, J=8.11 Hz, 1H) 4.20 (dd, J=8.33, 2.63 Hz, 1H) 3.11 (s, 3H) 3.14 (s, 3H) 1.73-1.86 (m, 2H) 0.85 (t, J=7.45 Hz, 3H).

### Step-4: Synthesis of (S)-3-(4-(dimethylamino)-6-((S)-1-(4-phenoxyphenyl)ethylamino)-1,3,5-triazin-2-yl)-4-ethyloxazolidin-2-one

In a microwave vial charged with (S)-1-(4-phenoxyphenyl)ethanamine hydrochloride (0.15 g, 0.7 mmol, 1.0 eq.), (S)-3-(4-chloro-6-(dimethylamino)-1,3,5-triazin-2-yl)-4-ethyloxazolidin-2-one (0.19 g, 0.70 mmol, 1.0 eq.) and N, N-Diisopropylethylamine (0.15 mL, 1.4 mmol, 2.0 eq.), in DMSO (5 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.05 g 16%), UPLC-MS (Method 4): Rt 2.51, m/z 449.5 [M+1]⁺; ¹H NMR (400 MHz, DMSO-d₆) δ ppm 7.83 (dd, J=10.09, 8.33 Hz, 1H) 7.32-7.42 (m, 3H) 7.06-7.14 (m, 1H) 6.89-7.00 (m, 3H) 5.10 (dd, J=12.50, 7.24 Hz, 1H) 4.42-4.57 (m, 1H) 4.35 (dt, J=11.84, 8.55 Hz, 1H) 4.09 (td, J=8.88, 2.85 Hz, 1H) 3.01 (s, 2H) 3.05 (s, 3H) 1.69-1.85 (m, 1H) 1.50-1.61 (m, 1H) 1.37-1.49 (m, 3H) 0.82 (t, J=7.24 Hz, 2H) 0.72 (t, J=7.45 Hz, 1H).

### Example-45: Synthesis of (R)-4-((S)-1-fluoroethyl)-3-(4-methyl-6-((S)-1-(4-phenoxyphenyl)ethylamino)-1,3,5-triazin-2-yl)oxazolidin-2-one (Compound 1.64)

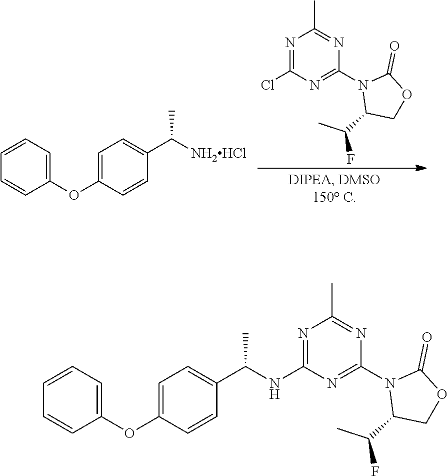

In a microwave vial charged with (S)-1-(4-phenoxyphenyl)ethanamine hydrochloride (0.06 g, 0.28 mmol, 1.0 eq.), (R)-3-(4-chloro-6-methyl-1,3,5-triazin-2-yl)-4-((S)-1-fluoroethyl)oxazolidin-2-one (0.073 g, 0.28 mmol, 1.0 eq.) and N, N-Diisopropylethylamine (0.1 mL, 0.52 mmol, 2.0 eq.), in DMSO (5 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as off white solid (0.055 g 44%), UPLC-MS (Method 6): Rt 5.51, m/z 438.5 [M+1]⁺; ¹H NMR (400 MHz, DMSO-d₆) δ ppm 8.63 (d, J=7.45 Hz, 1H) 7.30-7.44 (m, 3H) 7.08-7.16 (m, 1H) 6.88-7.02 (m, 3H) 4.99-5.08 (m, 1H) 4.77 (d, J=7.02 Hz, 1H) 4.57-4.70 (m, 2H) 4.33-4.44 (m, 2H) 2.26 (s, 3H) 1.40-1.48 (m, 2H) 1.28-1.38 (m, 1H) 1.14-1.26 (m, 3H).

### Example-46: Synthesis of (S)-4-methyl-3-(4-methyl-6-((S)-1-(4-phenoxyphenyl)ethylamino)-1,3,5-triazin-2-yl)oxazolidin-2-one (Compound 1.65)

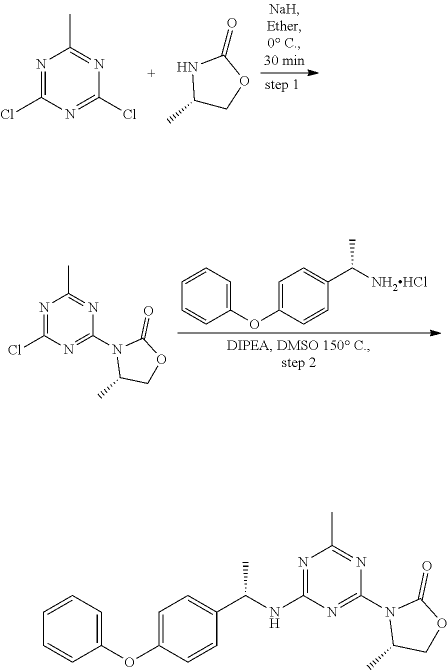

### Step-1: Synthesis of (S)-3-(4-chloro-6-methyl-1,3,5-triazin-2-yl)-4-methyloxazolidin-2-one

To a stirred solution of Sodium hydride (0.18 g, 4.5 mmol, 1.5 eq.) in Ether (10 mL) was added (S)-4-methyloxazolidin-2-one (0.34 g, 3.3 mmol, 1.1 eq.) in Ether (3 mL) at 0° C. The resulting solution stirred for 15 min, following this 2,4-dichloro-6-methyl-1,3,5-triazine (0.5 g, 3.0 mmol, 1.0 eq.) in Ether (2 mL) was added. The resulting mixture was stirred for another 30 min at same temperature. Following this, the reaction mixture diluted with saturated NH₄Cl (10 mL) and extracted with ethyl acetate (3×15 mL). The combined organic layers were washed with brine (30 mL), dried over anhydrous Na₂SO₄, filtered and concentrated under vacuum to get the solid residue which was purified by was purified normal phase silica-gel column chromatography provided title compound (0.25 g, 36.5%). LCMS: 228.9 [M+1]⁺; ¹H NMR (400 MHz, DMSO-d₆) δ ppm 4.71 (br. s., 1H) 4.50 (t, J=8.11 Hz, 1H) 4.11 (dd, J=8.33, 2.63 Hz, 1H) 2.53 (s, 3H) 1.40 (d, J=6.14 Hz, 3H).

### Step-2: Synthesis of (S)-4-methyl-3-(4-methyl-6-((S)-1-(4-phenoxyphenyl)ethylamino)-1,3,5-triazin-2-yl)oxazolidin-2-one

In a microwave vial charged with (S)-1-(4-phenoxyphenyl)ethanamine hydrochloride (0.1 g, 0.46 mmol, 1.0 eq.), (S)-3-(4-chloro-6-methyl-1,3,5-triazin-2-yl)-4-methyloxazolidin-2-one (0.107 g, 0.46 mmol, 1.0 eq.) and N, N-Diisopropylethylamine (0.12 mL, 0.93 mmol, 2.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.015 g 9%), UPLC-MS (Method 4): Rt 2.55, m/z 406.5 [M+1]⁺; ¹H NMR (400 MHz, DMSO-d₆) δ ppm 8.54 (d, J=7.89 Hz, 1H) 8.41 (d, J=8.77 Hz, 1H) 7.31-7.43 (m, 5H) 7.08-7.16 (m, 1H) 6.92-7.05 (m, 5H) 5.23-5.29 (m, 1H) 5.04-5.13 (m, 1H) 4.57-4.68 (m, 1H) 4.37-4.46 (m, 1H) 3.92-4.02 (m, 1H) 2.22-2.29 (m, 3H) 1.32-1.48 (m, 5H) 1.09 (d, J=6.14 Hz, 3H)

### Example-47: Synthesis of (S)-3-(4-((S)-1-(6-bromo-2-oxo-1,2-dihydroquinolin-3-yl)ethylamino)-6-methyl-1,3,5-triazin-2-yl)-4-ethyloxazolidin-2-one (Compound 1.66) and (S)-3-(4-((R)-1-(6-bromo-2-oxo-1,2-dihydroquinolin-3-yl)ethylamino)-6-methyl-1,3,5-triazin-2-yl)-4-ethyloxazolidin-2-one (Compound 1.67)

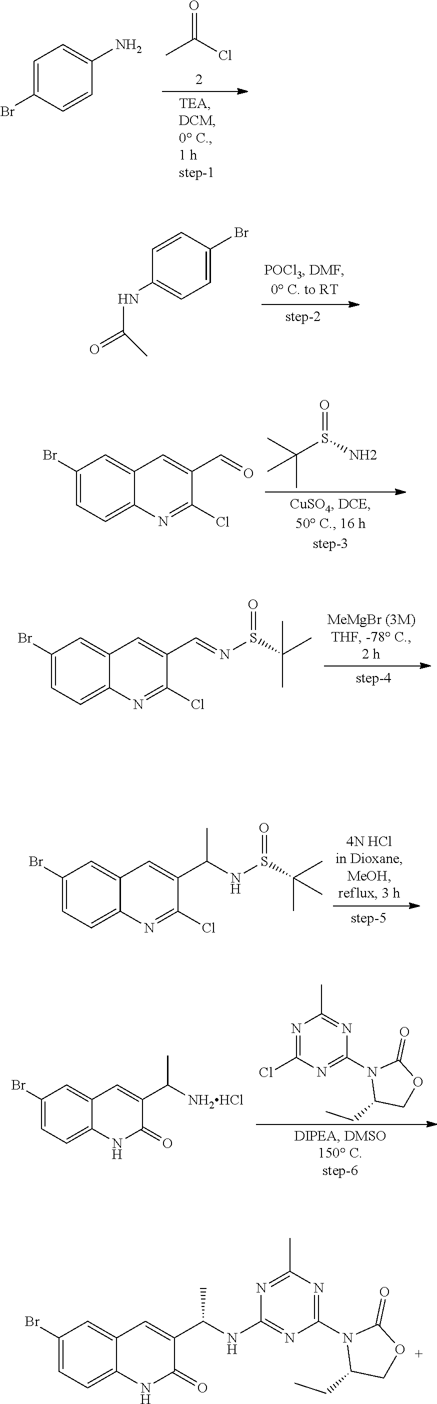

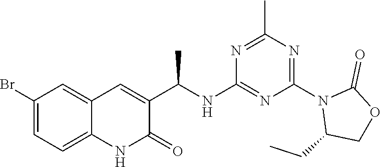 [SMILES: CC[C@H]1COC(=O)N1c1nc(C)nc(N[C@H](C)c2cc3cc(Br)ccc3[nH]c2=O)n1]

### Step-1: Synthesis of N-(4-bromophenyl)acetamide

To a stirred solution of 4-methoxyaniline (1.23 g, 10.0 mmol, 1.0 eq.) in DCM (20 mL) was added triethyl amine (4.15 mL, 30.0 mmol, 3.0 eq.). The resulting mixture was cooled to 0° C. and added acetyl chloride (1.17 mL, 15.0 mmol, 1.5 eq.) dropwise. The resulting mixture stirred for 1 h at same temperature. Following this, reaction mixture diluted with water (200 mL). The aqueous layer was separated extracted with DCM (3×30 mL). The combined organic layers were washed with brine (50 mL), dried over anhydrous Na₂SO₄, filtered and concentrated under vacuum to get title compound (1.3 g, 78%). LCMS: 166.1 [M+1]⁺; ¹H NMR (400 MHz, CHLOROFORM-d) δ ppm 7.35-7.43 (m, 2H) 7.26 (s, 1H) 7.09 (br. s., 1H) 6.83-6.91 (m, 2H) 3.79 (s, 3H) 2.15 (s, 3H).

### Step-2: Synthesis of 6-bromo-2-chloroquinoline-3-carbaldehyde

DMF (1.51 mL, 25.0 mmol, 2.5 eq.) was cooled to 0° C. and added POCl₃ (6.5 mL, 70.0 mmol, 7.0 eq.) drop wise over 5 min. To the above solution added N-(4-methoxyphenyl)acetamide (1.66 g, 10.0 mmol, 1.0 eq.) dissolved in DMF (2 mL). The resulting mixture heated at 100° C. for 16 h. Following this, reaction mixture diluted with ice cold water (100 mL) and stirred for 1 h. Filtered the solid and under vacuum to get title compound as half white solid (1.0 g, 45%). ¹H NMR (400 MHz, DMSO-d₆) δ ppm 10.37 (s, 1H) 8.85 (s, 1H) 7.96 (d, J=9.21 Hz, 1H) 7.70 (d, J=2.63 Hz, 1H) 7.62 (dd, J=9.21, 2.63 Hz, 1H) 3.89-3.95 (m, 3H).

### Step-3: Synthesis of (S,E)-N-((6-bromo-2-chloroquinolin-3-yl)methylene)-2-methylpropane-2-sulfinamide

To a stirred solution of 2-chloro-6-methoxyquinoline-3-carbaldehyde (1.0 g, 4.50 mmol, 1.0 eq.) and Copper (II) sulfate (1.43 g, 9.00 mmol, 2.0 eq.) in dichloroethane (20 mL) was added (S)-2-methylpropane-2-sulfinamide (2.18 g, 18.00 mmol, 4.0 eq.) at RT. The resulting mixture was heated at 50° C. for 16 h. Following this, reaction was allowed to cool to room temperature, filtered through celite pad, the celite pad washed with dichloromethane (30 mL). The combined filtrate dried over anhydrous Na₂SO₄ and concentrated under vacuum to get the solid residue which was purified by normal phase silica-gel column chromatography to get the title compound (1.25 g 85.7%). LCMS: 325.0 [M+1]⁺. ¹H NMR (400 MHz, DMSO-d₆) δ ppm 8.99 (s, 1H) 8.88 (s, 1H) 7.92 (d, J=9.21 Hz, 1H) 7.69 (d, J=2.63 Hz, 1H) 7.56 (dd, J=9.21, 3.07 Hz, 1H) 3.92 (s, 3H) 1.24-1.28 (m, 9H).

### Step-4: Synthesis of (S)—N-(1-(6-bromo-2-chloroquinolin-3-yl)ethyl)-2-methylpropane-2-sulfinamide

To a stirred solution of (S,E)-N-((2-chloro-6-methoxyquinolin-3-yl)methylene)-2-methylpropane-2-sulfinamide (1.2 g, 3.70 mmol, 1.0 eq.) in Ether (25 mL) was added drop wise 3 molar methylmagnesium bromide (6.8 mL, 18.50 mmol, 5.0 eq.) at −78° C. The resulting mixture was stirred for 2 h at same temperature and allowed to RT stirred for 1 h. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.65 g 51.6%). LCMS: 341.1 [M+1]⁺.

### Step-5: Synthesis of 3-(1-aminoethyl)-6-bromoquinolin-2(1H)-one hydrochloride

To a stirred solution of (S)—N-(1-(2-chloro-6-methoxyquinolin-3-yl)ethyl)-2-methylpropane-2-sulfinamide (0.25 g, 0.73 mmol, 1.0 eq.) in MeOH (2 mL) was added 4N HCl in dioxane (2.0 mL, 7.3 mmol, 10.0 eq.) at RT. The resulting mixture was heated to reflux for 3 h. Following this, the reaction mixture was evaporated under reduced pressure to get title compound which is used to next step without further purification (0.11 g 59.2%). LCMS: 255.1 [M+1]⁺.

### Step-6: Synthesis of (S)-4-ethyl-3-(4-((S)-1-(6-methoxy-2-oxo-1,2-dihydroquinolin-3-yl)ethylamino)-6-methyl-1,3,5-triazin-2-yl)oxazolidin-2-one and (S)-4-ethyl-3-(4-((R)-1-(6-methoxy-2-oxo-1,2-dihydroquinolin-3-yl)ethylamino)-6-methyl-1,3,5-triazin-2-yl)oxazolidin-2-one

In a microwave vial charged with 3-(1-aminoethyl)-6-bromoquinolin-2(1H)-one hydrochloride (0.1 g, 0.4 mmol, 1.0 eq.), (S)-3-(4-chloro-6-methyl-1,3,5-triazin-2-yl)-4-ethyloxazolidin-2-one (0.1 g, 0.40 mmol, 1.0 eq.) and N, N-Diisopropylethylamine (0.13 mL, 1.24 mmol, 3.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compounds as white solid. Compound 1.66 (0.058 g, 30%), UPLC-MS (Method 6): Rt 3.99, m/z 473.4 [M+1]⁺; ¹H NMR (400 MHz, DMSO-d₆) δ ppm 8.44 (d, J=7.02 Hz, 1H) 7.89 (d, J=2.19 Hz, 1H) 7.56-7.65 (m, 2H) 7.20-7.28 (m, 1H) 5.03-5.12 (m, 1H) 4.30-4.45 (m, 2H) 4.04 (d, J=5.70 Hz, 1H) 2.28 (s, 2H) 2.22 (s, 1H) 1.89 (s, 1H) 1.75-1.84 (m, 1H) 1.41 (d, J=7.02 Hz, 3H) 1.18-1.35 (m, 2H) 0.85 (t, J=7.45 Hz, 1H) 0.51 (t, J=7.45 Hz, 3H). Compound 1.67 (0.02 g, 13%), UPLC-MS (Method 6): Rt 4.23, m/z 473.4 [M+1]⁺; ¹H NMR (400 MHz, DMSO-d₆) δ ppm 11.93 (br. s., 1H) 8.40 (d, J=7.45 Hz, 1H) 8.23 (d, J=7.89 Hz, 1H) 7.88 (d, J=2.19 Hz, 1H) 7.71-7.78 (m, 1H) 7.57-7.64 (m, 1H) 7.20-7.28 (m, 1H) 5.24-5.36 (m, 1H) 5.12-5.24 (m, 1H) 4.58 (br. s., 1H) 4.41 (t, J=8.33 Hz, 1H) 4.23-4.35 (m, 2H) 4.17 (d, J=5.70 Hz, 1H) 4.05-4.12 (m, 1H) 2.20-2.30 (m, 3H) 1.64-1.86 (m, 3H) 1.40 (d, J=6.58 Hz, 3H) 0.79-0.89 (m, 3H).

### Example-48: Synthesis of (S)-4-ethyl-3-(4-((S)-1-(5-(4-fluoro-3-methylphenyl)pyridin-2-yl)ethylamino)-6-methyl-1,3,5-triazin-2-yl)oxazolidin-2-one (Compound 1.68)

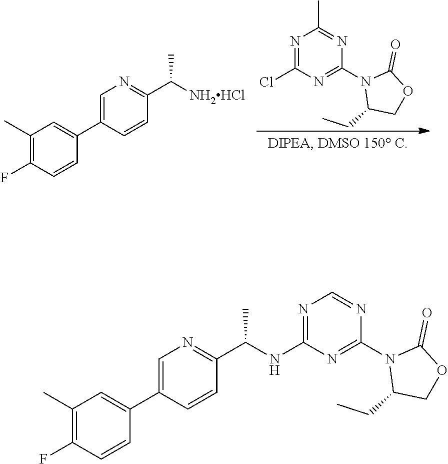

In a microwave vial charged with (S)-1-(5-(4-fluoro-3-methylphenyl)pyridin-2-yl)ethanamine hydrochloride (0.15 g, 0.65 mmol, 1.0 eq.), (S)-3-(4-chloro-6-methyl-1,3,5-triazin-2-yl)-4-ethyloxazolidin-2-one (0.17 g, 0.72 mmol, 1.1 eq.) and N, N-Diisopropylethylamine (0.2 mL, 1.3 mmol, 2.0 eq.), in DMSO (5 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.102 g 36%), UPLC-MS (Method 6): Rt 4.20, m/z 437.5 [M+1]⁺; ¹H NMR (400 MHz, DMSO-d₆) δ ppm 8.74-8.83 (m, 1H) 8.54 (d, J=7.45 Hz, 1H) 8.36 (d, J=7.89 Hz, 1H) 7.94-8.04 (m, 2H) 7.59-7.67 (m, 1H) 7.44-7.58 (m, 2H) 7.39 (d, J=8.33 Hz, 1H) 7.21-7.32 (m, 2H) 5.29-5.37 (m, 1H) 5.08-5.17 (m, 1H) 4.57 (d, J=6.58 Hz, 1H) 4.30-4.46 (m, 3H) 4.11-4.19 (m, 1H) 4.06 (dd, J=8.33, 2.63 Hz, 1H) 2.20-2.32 (m, 7H) 1.76-1.84 (m, 1H) 1.50 (d, J=7.02 Hz, 4H) 1.20-1.37 (m, 4H) 0.85 (t, J=7.45 Hz, 2H) 0.58 (t, J=7.24 Hz, 3H).

### Example-49: Synthesis of (S)-3-(4-((S)-1-(6-chloroimidazo[1,2-b]pyridazin-3-yl)ethylamino)-6-methyl-1,3,5-triazin-2-yl)-4-ethyloxazolidin-2-one (Compound 1.69) and (S)-3-(4-((R)-1-(6-chloroimidazo[1,2-b]pyridazin-3-yl)ethylamino)-6-methyl-1,3,5-triazin-2-yl)-4-ethyloxazolidin-2-one (Compound 1.70)

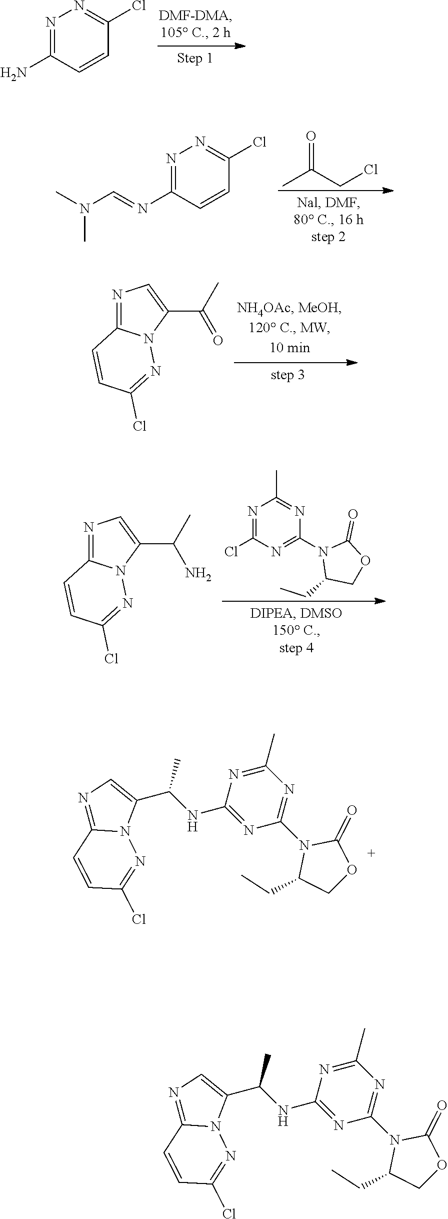

### Step-1: Synthesis of (E)-N′-(6-chloropyridazin-3-yl)-N,N-dimethylformimidamide

To a stirred solution of 6-chloropyridazin-3-amine (2.0 g, 15.4 mmol, 1.0 eq.) in 1,1-dimethoxy-N,N-dimethylmethanamine (2.46 mL, 18.5 mmol, 1.2 eq.) was heated at 105° C. for 2 h. On completion of starting material, the reaction mixture was cooled to RT and concentrated to give title compound (3.1 g crude). LCMS: 184.9 [M+1]⁺; ¹H NMR (400 MHz, CHLOROFORM-d) δ ppm 8.54 (s, 1H) 7.24-7.31 (m, 1H) 7.05 (d, J=9.21 Hz, 1H) 3.10 (s, 3H) 3.13 (s, 3H)

### Step-2: Synthesis of 1-(6-chloroimidazo[1,2-b]pyridazin-3-yl)ethanone

To a stirred solution of (E)-N′-(6-chloropyridazin-3-yl)-N,N-dimethylformimidamide (2.5 g, 13.54 mmol, 1.0 eq.) in DMF (60 mL) was added NaI (2.02 g, 24.36 mmol, 1.0 eq.) and chloroacetone (1.96 mL, 24.3 mmol, 1.8 eq.). The mixture was heated at 80° C. overnight and then concentrated under reduced pressure. The residue was purified by column chromatography to get title compound (0.6 g, 22%). LCMS: 196.0 [M+1]⁺; ¹H NMR (400 MHz, CHLOROFORM-d) δ ppm 8.41 (s, 1H) 8.02-8.07 (m, 1H) 7.29 (d, J=9.65 Hz, 1H) 2.76 (s, 3H).

### Step-3: Synthesis of 1-(6-chloroimidazo[1,2-b]pyridazin-3-yl)ethanamine

In a microwave vial charged with 1-(6-chloroimidazo[1,2-b]pyridazin-3-yl)ethanone (0.2 g, 1.02 mmol, 1.0 eq.), Ammonium acetate (0.78 g, 10.22 mmol, 10.0 eq.) and sodium cyanoborohydride (0.064 g, 1.02 mmol, 1.02 eq.), in MeOH (10 mL). The resulting mixture was heated at 120° C. for 10 min. Following this, the reaction mixture was allowed to cool to RT, basified with 6N NaOH until pH˜10 and extracted with EtOAc (3×15 mL). The combined organic layers were washed with brine (25 mL), dried over Na₂SO₄ and concentrated under reduced pressure to get the title compound which was carried forward without any further purification (0.12 g 60%). LCMS: 197.0 [M+1]⁺

### Step-4: Synthesis of (S)-3-(4-((S)-1-(6-chloroimidazo[1,2-b]pyridazin-3-yl)ethylamino)-6-methyl-1,3,5-triazin-2-yl)-4-ethyloxazolidin-2-one and (S)-3-(4-((R)-1-(6-chloroimidazo[1,2-b]pyridazin-3-yl)ethylamino)-6-methyl-1,3,5-triazin-2-yl)-4-ethyloxazolidin-2-one

In a microwave vial charged with 1-(6-chloroimidazo[1,2-b]pyridazin-3-yl)ethanamine (0.1 g, 0.5 mmol, 1.0 eq.), (S)-3-(4-chloro-6-methyl-1,3,5-triazin-2-yl)-4-ethyloxazolidin-2-one (0.12 g, 0.5 mmol, 1.0 eq.) and N, N-Diisopropylethylamine (0.27 mL, 1.24 mmol, 3.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compounds as white solid. Compound 1.69 (0.020 g, 10%), UPLC-MS (Method 6): Rt 2.92, m/z 403.4 [M+1]⁺; ¹H NMR (400 MHz, DMSO-d₆) δ ppm 8.63 (d, J=7.45 Hz, 1H) 8.45 (d, J=8.33 Hz, 1H) 8.24 (d, J=9.21 Hz, 1H) 7.83 (s, 1H) 7.73 (s, 1H) 7.39 (d, J=9.65 Hz, 1H) 5.66-5.78 (m, 1H) 5.56 (dt, J=14.03, 7.02 Hz, 1H) 4.57 (br. s., 1H) 4.31-4.50 (m, 2H) 4.04-4.20 (m, 2H) 2.28 (s, 3H) 1.73-1.83 (m, 1H) 1.57-1.64 (m, 3H) 1.33-1.42 (m, 2H) 0.83 (t, J=7.24 Hz, 1H) 0.53 (t, J=7.24 Hz, 2H). Compound 1.70 (0.02 g, 10%), UPLC-MS (Method 6): Rt 3.10, m/z 403.4 [M+1]⁺; ¹H NMR (400 MHz, DMSO-d₆) δ ppm 8.65 (d, J=7.45 Hz, 2H) 8.28 (d, J=9.21 Hz, 1H) 7.89 (s, 1H) 7.47 (d, J=9.65 Hz, 1H) 5.59 (s, 2H) 4.32-4.61 (m, 4H) 4.12-4.19 (m, 3H) 2.25-2.31 (m, 3H) 1.61 (d, J=7.02 Hz, 3H) 0.79-0.87 (m, 3H).

### Example-50: Synthesis of (S)-3-(4-cyclopropyl-6-((S)-1-(4-phenoxyphenyl)ethylamino)-1,3,5-triazin-2-yl)-4-ethyloxazolidin-2-one (Compound 1.71)

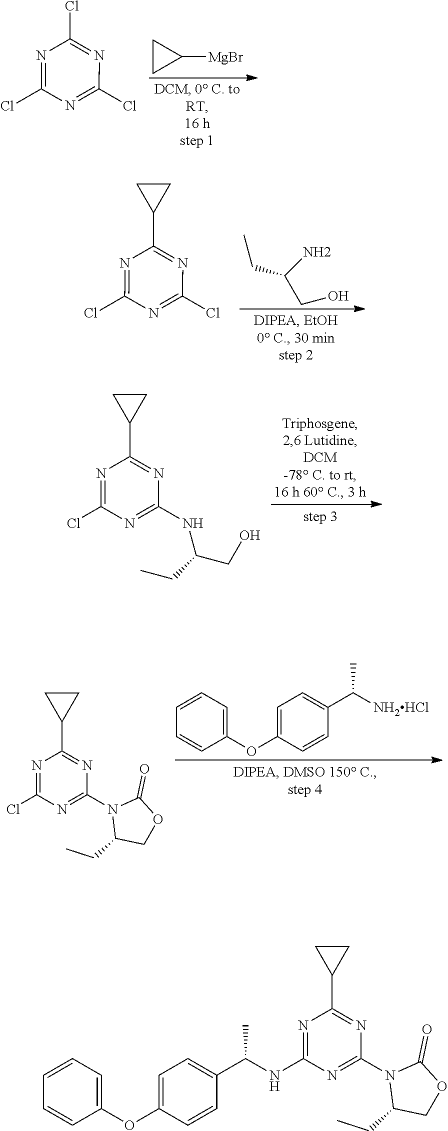

### Step-1: Synthesis of 2,4-dichloro-6-cyclopropyl-1,3,5-triazine

To a stirred solution of 2,4,6-trichloro-1,3,5-triazine (2.5 g, 13.0 mmol, 1.0 eq.) in DCM (30 mL) was added Cyclopropylmagnesium bromide solution (54 mL, 27.0 mmol, 2.0 eq.) at 0° C. The reaction mixture allowed to RT and stirred for 16 h. On completion of starting material, quenched with saturated ammonium chloride solution (20 mL) and extracted with DCM (3×30 mL). Combined organics were washed with water (40 mL), brine (40 mL), dried over Na2SO4, filtered and concentrated under vacuum to get the solid residue which was purified by was purified normal phase silica-gel column to get the title compound as white solid (2 g 81%). ¹H NMR (400 MHz, DMSO-d₆) δ ppm 1.33 (td, J=7.67, 3.95 Hz, 2H) 1.17-1.25 (m, 2H).

### Step-2: Synthesis of (S)-2-(4-chloro-6-cyclopropyl-1,3,5-triazin-2-ylamino)butan-1-ol

To a stirred solution of 2,4-dichloro-6-cyclopropyl-1,3,5-triazine (2 g, 10.0 mmol, 1.0 eq.), (S)-2-aminobutan-1-ol (1.03 g, 11.0 mmol, 1.1 eq.) in EtOH (20 mL) was added DIPEA (3.4 mL, 20.0 mmol, 2.0 eq.) at 0° C. The resulting mixture was stirred for 30 min. Following this, concentrated under vacuum to get the solid residue which was purified by was purified normal phase silica-gel column to get the title compound as brown color semi solid (1.5 g 62%). LCMS: 243.1 [M+1]⁺; ¹H NMR (400 MHz, DMSO-d₆) δ ppm 8.13 (br. s., 1H) 4.67 (br. s., 1H) 4.03 (q, J=7.31 Hz, 1H) 3.75-3.90 (m, 1H) 3.35-3.42 (m, 2H) 1.81-1.91 (m, 1H) 1.60 (dd, J=12.94, 5.48 Hz, 1H) 1.33-1.44 (m, 1H) 1.17 (t, J=7.02 Hz, 1H) 0.97-1.08 (m, 4H) 0.83 (t, J=7.45 Hz, 3H)

### Step-3: Synthesis of (S)-3-(4-chloro-6-cyclopropyl-1,3,5-triazin-2-yl)-4-ethyloxazolidin-2-one

To a stirred solution of (S)-2-(4-chloro-6-cyclopropyl-1,3,5-triazin-2-ylamino)butan-1-ol (1.5 g, 6.0 mmol, 1.0 eq.), 2,6 lutidine (3.11 mL, 4.5 mmol, 4.5 eq.) in DCM (20 mL) was added Triphosgene (1.47 g, 4.9 mmol, 0.8 eq.) at −78° C. The resulting mixture was stirred for 10 min at same temperature then allowed to RT and stirred for 16 h. Following this, the reaction mixture hated to 60° C. and stirred for 3 h. On completion of starting material, cool the reaction mixture quenched with saturated ammonium chloride solution (10 mL) and extracted with DCM (3×20 mL). Combined organics were washed with water (20 mL), brine (30 mL), dried over Na2SO4, filtered and concentrated under vacuum to get the solid residue which was purified by was purified normal phase silica-gel column to get the title compound as brown oil (0.8 g 49%). LCMS: 269.1 [M+1]⁺; ¹H NMR (400 MHz, DMSO-d₆) δ ppm 4.54-4.64 (m, 1H) 4.45 (t, J=8.33 Hz, 1H) 4.25 (dd, J=8.77, 2.63 Hz, 1H) 4.03 (q, J=7.31 Hz, 1H) 2.41 (s, 2H) 2.10 (ddd, J=12.39, 8.00, 4.60 Hz, 1H) 1.72-1.85 (m, 2H) 1.08-1.26 (m, 5H) 0.86 (t, J=7.45 Hz, 3H).

### Step-4: Synthesis of (S)-3-(4-cyclopropyl-6-((S)-1-(4-phenoxyphenyl)ethylamino)-1,3,5-triazin-2-yl)-4-ethyloxazolidin-2-one

In a microwave vial charged with (S)-1-(4-phenoxyphenyl)ethanamine hydrochloride (0.12 g, 0.56 mmol, 1.0 eq.), (S)-3-(4-chloro-6-cyclopropyl-1,3,5-triazin-2-yl)-4-ethyloxazolidin-2-one (0.15 g, 0.56 mmol, 1.0 eq.) and N, N-Diisopropylethylamine (0.19 mL, 1.12 mmol, 2.0 eq.), in DMSO (3 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid (0.046 g 19%), UPLC-MS (Method 7): Rt 4.11, m/z 446.5 [M+1]⁺; ¹H NMR (400 MHz, DMSO-d₆) δ ppm 8.43 (d, J=7.89 Hz, 1H) 8.33 (d, J=8.33 Hz, 1H) 7.32-7.42 (m, 6H) 7.08-7.15 (m, 2H) 6.90-7.00 (m, 6H) 5.10 (dq, J=14.63, 7.25 Hz, 2H) 4.44-4.56 (m, 2H) 4.37 (dt, J=12.06, 8.22 Hz, 2H) 4.08-4.16 (m, 2H) 1.68-1.85 (m, 3H) 1.38-1.60 (m, 7H) 0.89-1.04 (m, 6H) 0.79-0.89 (m, 2H) 0.73 (t, J=7.45 Hz, 3H).

### Example-51: Synthesis of (S)-3-(4-((R)-1-(4-(benzyloxy)phenyl)ethylamino)-6-methyl-1,3,5-triazin-2-yl)-4-ethyloxazolidin-2-one (Compound 1.72) and (S)-3-(4-((S)-1-(4-(benzyloxy)phenyl)ethylamino)-6-methyl-1,3,5-triazin-2-yl)-4-ethyloxazolidin-2-one (Compound 1.73)

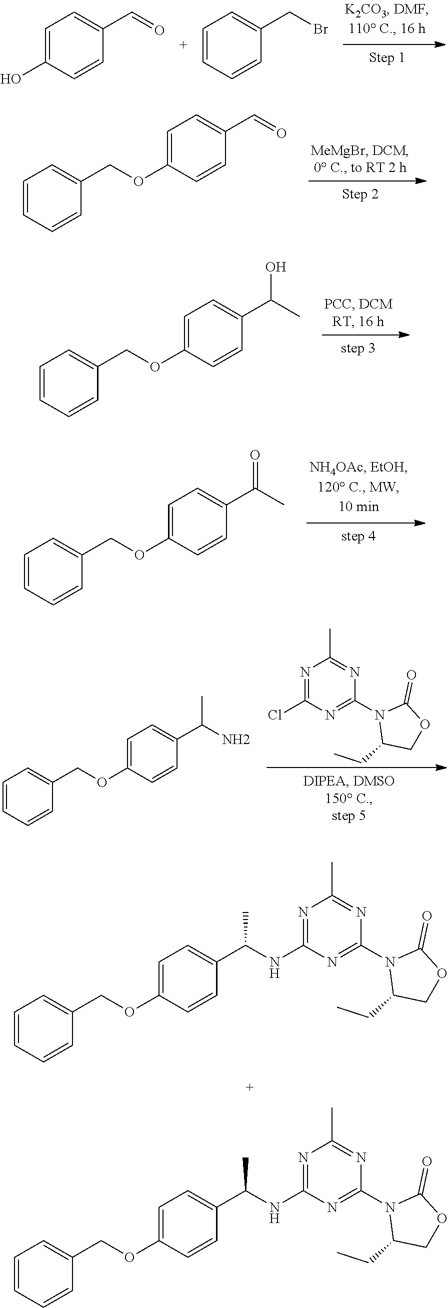

### Step-1: Synthesis of 4-(benzyloxy)benzaldehyde

To a stirred solution of 4-hydroxybenzaldehyde (2 g, 16.2 mmol, 1.0 eq.) and (bromomethyl)benzene (3.0 g, 18.02 mmol, 1.1 eq.) in DMF (20 mL) was added K₂CO₃ (4.4 g, 32.4 mmol, 2 eq.) The resulting mixture heated at 110° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound as white solid (21 g 58%). ¹H NMR (400 MHz, CHLOROFORM-d) δ ppm 9.89 (s, 1H) 7.84 (m, J=8.33 Hz, 2H) 7.33-7.47 (m, 5H) 7.08 (m, J=8.77 Hz, 2H) 5.16 (s, 2H).

### Step-2: Synthesis of 1-(4-(benzyloxy)phenyl)ethanol

To a stirred solution of 4-(benzyloxy)benzaldehyde (1.0 g, 4.71 mmol, 1.0 eq.) in DCM (10 mL) was added drop wise 3 molar methylmagnesium bromide (2.09 mL, 7.06 mmol, 1.5 eq.) at 0° C. The resulting mixture allowed to RT and stirred for 2 h. The reaction was then quenched by careful addition of saturated NH₄Cl (10 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.8 g 74%). ¹H NMR (400 MHz, DMSO-d₆) δ ppm 7.28-7.47 (m, 5H) 7.24 (m, J=8.33 Hz, 2H) 6.94 (m, J=8.77 Hz, 2H) 5.08 (s, 2H) 5.01 (d, J=4.38 Hz, 1H) 4.62-4.69 (m, 1H) 1.28 (d, J=6.58 Hz, 3H)

### Step-3: Synthesis of 1-(4-(benzyloxy)phenyl)ethanone

To a stirred solution of 1-(4-(benzyloxy)phenyl)ethanol (0.8 g, 3.5 mmol, 1.0 eq.) in DCM (10 mL) was added PCC (1.13 g, 5.26 mmol, 1.5 eq.) at RT. The resulting mixture was stirred for 16 h. on the completion of starting material, the reaction mixture filtered and evaporated under reduced pressure to get solid. This solid washed with ether and evaporated to give title compound (0.65 g 82%). LCMS: 226.7 [M+1]⁺; ¹H NMR (400 MHz, CHLOROFORM-d) δ ppm 7.94 (m, J=8.77 Hz, 2H) 7.30-7.50 (m, 5H) 7.26 (s, 1H) 7.01 (m, J=8.77 Hz, 2H) 5.14 (s, 2H) 2.56 (s, 3H)

### Step-4: Synthesis of 1-(4-(benzyloxy)phenyl)ethanamine

In a microwave vial charged with 1-(4-(benzyloxy)phenyl)ethanone (0.4 g, 1.76 mmol, 1.0 eq.), Ammonium acetate (1.36 g, 17.6 mmol, 10.0 eq.) and sodium cyanoborohydride (0.10 g, 1.76 mmol, 1.0 eq.), in EtOH (10 mL). The resulting mixture was heated at 120° C. for 10 min. Following this, the reaction mixture was allowed to cool to RT, basified with 6N NaOH until pH˜10 and extracted with EtOAc (3×10 mL). The combined organic layers were washed with brine (15 mL), dried over Na₂SO₄ and concentrated under reduced pressure to get the title compound as semi solid which was carried forward without any further purification (0.25 g 62%). LCMS: 127.9 [M+1]+

### Step-5: Synthesis of (S)-3-(4-((S)-1-(4-(benzyloxy)phenyl)ethylamino)-6-methyl-1,3,5-triazin-2-yl)-4-ethyloxazolidin-2-one and (S)-3-(4-((R)-1-(4-(benzyloxy)phenyl)ethylamino)-6-methyl-13,5-triazin-2-yl)-4-ethyloxazolidin-2-one

In a microwave vial charged with 1-(4-(benzyloxy)phenyl)ethanamine (0.25 g, 1.1 mmol, 1.0 eq.) and (S)-3-(4-chloro-6-methyl-1,3,5-triazin-2-yl)-4-ethyloxazolidin-2-one (0.266 g, 1.1 mmol, 1.0 eq.) in DMSO (2 mL) was added N, N-Diisopropylethylamine (0.4 mL, 2.2 mmol, 2.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound as off white solid Compound 1.72 (0.032 g, 6%), UPLC-MS (Method 4): Rt 2.58, m/z 434.5 [M+1]⁺; ¹H NMR (400 MHz, DMSO-d₆) δ ppm 8.48 (d, J=8.33 Hz, 1H) 7.21-7.47 (m, 6H) 6.89-6.97 (m, 2H) 5.00-5.08 (m, 3H) 4.47-4.60 (m, 1H) 4.33-4.42 (m, 1H) 4.10-4.17 (m, 1H) 2.21-2.27 (m, 3H) 1.84 (s, 1H) 1.51-1.59 (m, 1H) 1.37-1.48 (m, 3H) 0.82 (t, J=7.45 Hz, 1H) 0.72 (t, J=7.45 Hz, 2H), Compound 1.73 (0.011 g, 3%), UPLC-MS (Method 4): Rt 2.61, m/z 434.5 [M+1]⁺; ¹H NMR (400 MHz, DMSO-d₆) δ ppm 8.45 (d, J=8.33 Hz, 1H) 7.26-7.46 (m, 7H) 6.91-6.97 (m, 2H) 5.16-5.25 (m, 1H) 4.98-5.09 (m, 3H) 4.52-4.59 (m, 1H) 4.33-4.44 (m, 2H) 4.14 (d, J=6.14 Hz, 1H) 2.20-2.27 (m, 3H) 1.72-1.92 (m, 3H) 1.40 (d, J=7.02 Hz, 3H) 0.78-0.90 (m, 3H)

### Example-52: Synthesis of (S)-4-ethyl-3-(4-methyl-6-((S)-1-(4-(pyridin-3-yloxy)phenyl)ethylamino)-1,3,5-triazin-2-yl)oxazolidin-2-one (Compound 1.74)

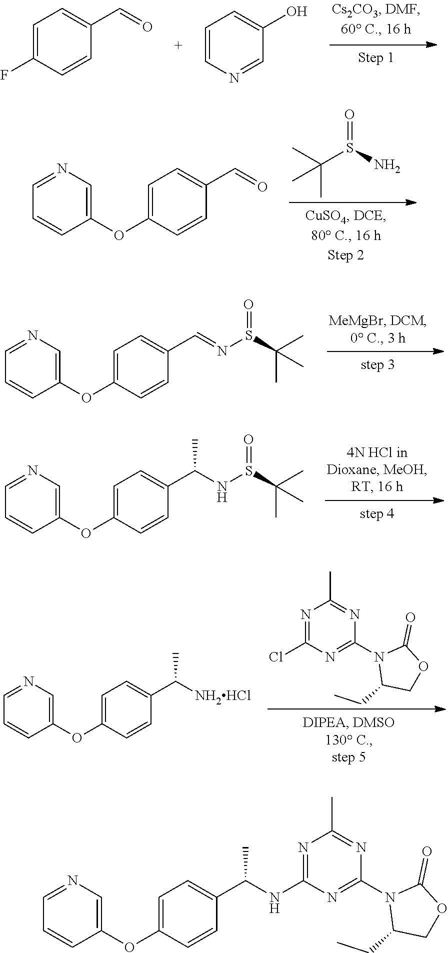

### Step-1: Synthesis of 4-(pyridin-3-yloxy)benzaldehyde

To a stirred solution of 4-fluorobenzaldehyde (1.3 g, 10.0 mmol, 1.0 eq.) and pyridin-3-ol (1.0 g, 10.0 mmol, 1.0 eq.) in DMF (10 mL) was added Cs₂CO₃ (3.4 g, 10.0 mmol, 1 eq.) The resulting mixture heated at 60° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound (0.68 g, 34%). LCMS: 200.0 [M+1]⁺; ¹H NMR (400 MHz, DMSO-d₆) δ ppm 9.93 (s, 1H) 8.45-8.51 (m, 2H) 7.94 (m, J=8.77 Hz, 2H) 7.59-7.67 (m, 1H) 7.50 (dd, J=8.33, 4.39 Hz, 1H) 7.18 (m, J=8.33 Hz, 2H).

### Step-2: Synthesis of (R,E)-2-methyl-N-(4-(pyridin-3-yloxy)benzylidene)propane-2-sulfinamide

To a stirred solution of 4-(pyridin-3-yloxy)benzaldehyde (0.67 g, 3.35 mmol, 1.0 eq.) and Copper(II) sulfate (1.3 g, 8.37 mmol, 2.5 eq.) in dichloroethane (15 mL) was added (R)-2-methylpropane-2-sulfinamide (0.81 g, 6.7 mmol, 2.0 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound (0.89 g, 89%). LCMS: 303.2 [M+1]⁺; ¹H NMR (400 MHz, DMSO-d₆) δ ppm 8.53 (s, 1H) 8.48 (br. s., 1H) 7.99 (m, J=8.77 Hz, 2H) 7.58-7.63 (m, 1H) 7.47-7.53 (m, 1H) 7.16 (m, J=8.77 Hz, 2H) 5.75 (s, 1H) 1.18 (s, 9H).

### Step-3: Synthesis of (R)-2-methyl-N—((S)-1-(4-(pyridin-3-yloxy)phenyl)ethyl)propane-2-sulfinamide

To a stirred solution of (R,E)-2-methyl-N-(4-(pyridin-3-yloxy)benzylidene)propane-2-sulfinamide (0.89 g, 2.94 mmol, 1.0 eq.) in DCM (10 mL) was added drop wise 3 molar methylmagnesium bromide (3.0 mL, 11.78 mmol, 4.0 eq.) at 0° C. The resulting mixture was stirred for 30 min at same temperature then allowed to RT and stirred for 3 h. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.6 g 64%). LCMS: 319.2 [M+1]⁺; ¹H NMR (400 MHz, DMSO-d₆) δ ppm 8.31-8.41 (m, 2H) 7.37-7.44 (m, 4H) 7.02 (d, J=8.33 Hz, 2H) 5.35 (d, J=5.26 Hz, 1H) 4.38-4.45 (m, 1H) 1.46 (d, J=6.58 Hz, 3H) 1.10-1.12 (m, 9H).

### Step-4: Synthesis of (S)-1-(4-(pyridin-3-yloxy)phenyl)ethanamine hydrochloride

To a stirred solution of (R)-2-methyl-N—((S)-1-(4-(pyridin-3-yloxy)phenyl)ethyl)propane-2-sulfinamide (0.6 g, 1.8 mmol, 1.0 eq.) in methanol (3 mL) was added 4N HCl in dioxane (3 mL) at RT. The resulting mixture was stirred for 16 h. Following this, the reaction mixture was evaporated under reduced pressure to get solid. This solid washed with ether and evaporated to give title compound (0.5 g crude). LCMS: 215.1 [M+1]⁺;

### Step-5: Synthesis of (S)-4-ethyl-3-(4-methyl-6-((S)-1-(4-(pyridin-3-yloxy)phenyl)ethylamino)-1,3,5-triazin-2-yl)oxazolidin-2-one

In a microwave vial charged with (S)-1-(4-(pyridin-3-yloxy)phenyl)ethanamine hydrochloride (0.5 g, 2.33 mmol, 1.0 eq.) and (S)-3-(4-chloro-6-methyl-1,3,5-triazin-2-yl)-4-ethyloxazolidin-2-one (0.84 g, 3.5 mmol, 1.5 eq.) in DMSO (3 mL) was added N, N-Diisopropylethylamine (1.2 mL, 6.99 mmol, 3.0 eq.) at RT. The resulting mixture was heated at 130° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound as white solid (0.038 g, 5%), UPLC-MS (Method 2): Rt 2.26, m/z 421.5 [M+1]⁺; ¹H NMR (400 MHz, DMSO-d₆) δ ppm 8.53 (d, J=7.89 Hz, 1H) 8.31-8.46 (m, 2H) 7.37-7.46 (m, 4H) 6.96-7.08 (m, 2H) 5.23-5.32 (m, 1H) 5.08 (quin, J=7.13 Hz, 1H) 4.33-4.44 (m, 2H) 4.11-4.18 (m, 1H) 2.22-2.27 (m, 3H) 1.72-1.92 (m, 2H) 1.44 (d, J=7.02 Hz, 3H) 0.79-0.90 (m, 3H).

### Example-53: Synthesis of (S)-4-ethyl-3-(4-methyl-6-((S)-1-(5-phenoxypyridin-2-yl)ethylamino)-1,3,5-triazin-2-yl)oxazolidin-2-one (Compound 1.75) and (S)-4-ethyl-3-(4-methyl-6-((R)-1-(5-phenoxypyridin-2-yl)ethylamino)-1,3,5-triazin-2-yl)oxazolidin-2-one (Compound 1.76)

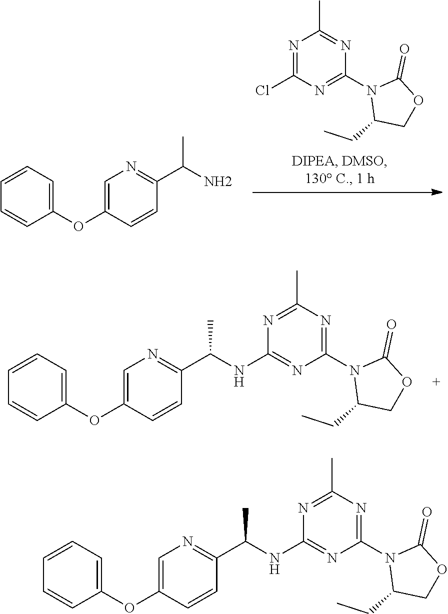

In a microwave vial charged with 1-(5-phenoxypyridin-2-yl)ethanamine (0.12 g, 0.56 mmol, 1.0 eq.), (S)-3-(4-chloro-6-methyl-1,3,5-triazin-2-yl)-4-ethyloxazolidin-2-one (0.13 g, 0.56 mmol, 1.0 eq.) in DMSO (5 mL) was added N, N-Diisopropylethylamine (0.2 g, 1.12 mmol, 2.0 eq.) at RT. The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds. Compound 1.75 (0.002 g, 1%), UPLC-MS (Method 4): Rt 2.36, m/z 421.5 [M+1]⁺; ¹H NMR (400 MHz, DMSO-d₆) δ ppm 8.55 (d, J=7.02 Hz, 1H) 8.25-8.37 (m, 1H) 7.33-7.45 (m, 3H) 7.11-7.20 (m, 1H) 6.95-7.06 (m, 2H) 5.19-5.34 (m, 1H) 5.05-5.19 (m, 1H) 4.55 (br. s., 1H) 4.31-4.49 (m, 2H) 4.06-4.18 (m, 1H) 2.20-2.29 (m, 2H) 1.46 (d, J=7.02 Hz, 2H) 1.29-1.39 (m, 1H) 0.82 (t, J=7.45 Hz, 1H) 0.64 (t, J=7.24 Hz, 2H), Compound 1.76 (0.002 g, 1%), UPLC-MS (Method 4): Rt 2.37, m/z 421.5 [M+1]⁺; ¹H NMR (400 MHz, DMSO-d₆) δ ppm 8.45 (d, J=7.45 Hz, 1H) 8.30 (d, J=2.63 Hz, 2H) 7.36-7.47 (m, 4H) 7.14-7.22 (m, 1H) 7.00-7.09 (m, 2H) 5.09-5.17 (m, 1H) 4.25-4.44 (m, 3H) 4.09-4.19 (m, 2H) 2.22-2.28 (m, 3H) 1.69-1.87 (m, 3H) 1.47 (d, J=7.02 Hz, 3H) 0.79-0.89 (m, 4H)

### Example-54: Synthesis of (S)-4-ethyl-3-(4-methyl-6-((S)-1-(4-(pyridin-3-yloxy)phenyl)ethylamino)-1,3,5-triazin-2-yl)oxazolidin-2-one (Compound 1.77)

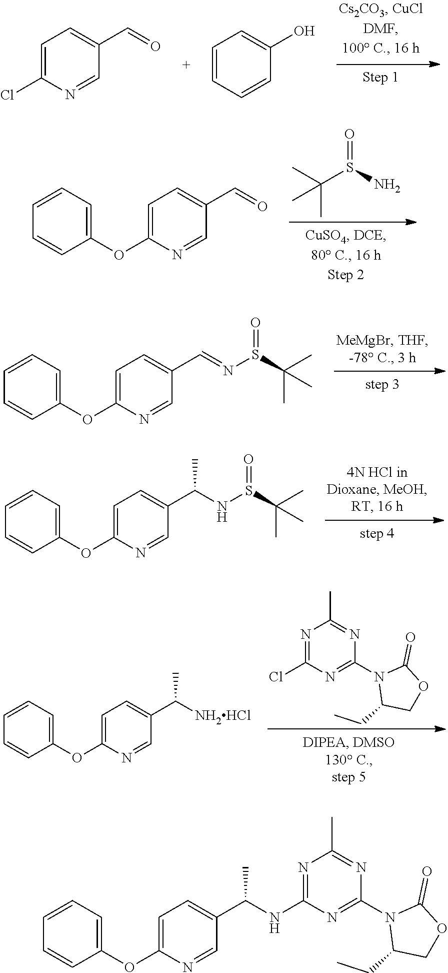

### Step-1: Synthesis of 6-phenoxynicotinaldehyde

To a stirred solution of 6-chloronicotinaldehyde (2.0 g, 14.1 mmol, 1.0 eq.) and phenol (1.32 g, 14.1 mmol, 1.0 eq.) in DMF (20 mL) was added Cs₂CO₃ (5.5 g, 16.9 mmol, 1.2 eq.) and CuCl (1.61 g, 16.9 mmol, 1.2 eq.). The resulting mixture heated at 100° C. for 10 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound (2.8 g, 99%). LCMS: 200.0 [M+1]⁺; ¹H NMR (400 MHz, DMSO-d₆) δ ppm 9.99 (s, 1H) 8.70 (d, J=2.19 Hz, 1H) 8.27 (dd, J=8.55, 2.41 Hz, 1H) 7.43-7.52 (m, 2H) 7.18-7.32 (m, 4H).

### Step-2: Synthesis of (R,E)-2-methyl-N-((6-phenoxypyridin-3-yl)methylene)propane-2-sulfinamide

To a stirred solution of 6-phenoxynicotinaldehyde (1.5 g, 7.5 mmol, 1.0 eq.) and Copper(II) sulfate (2.9 g, 18.8 mmol, 2.5 eq.) in dichloroethane (10 mL) was added (R)-2-methylpropane-2-sulfinamide (1.3 g, 11.2 mmol, 1.8 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound (1.0 g, 44%). LCMS: 303.0 [M+1]⁺; ¹H NMR (400 MHz, DMSO-d₆) δ ppm 8.68 (d, J=2.19 Hz, 1H) 8.58 (s, 1H) 8.37 (dd, J=8.77, 2.19 Hz, 1H) 7.43-7.50 (m, 2H) 7.16-7.30 (m, 4H) 1.18 (s, 9H).

### Step-3: Synthesis of (R)-2-methyl-N—((S)-1-(6-phenoxypyridin-3-yl)ethyl)propane-2-sulfinamide

To a stirred solution of (R,E)-2-methyl-N-((6-phenoxypyridin-3-yl)methylene)propane-2-sulfinamide (1.0 g, 3.3 mmol, 1.0 eq.) in THF (10 mL) was added drop wise 3 molar methylmagnesium bromide (1.7 mL, 4.9 mmol, 1.5 eq.) at −78° C. The resulting mixture was stirred for 3 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.6 g 57%). LCMS: 319.0 [M+1]⁺;

### Step-4: Synthesis of (S)-1-(6-phenoxypyridin-3-yl)ethanamine hydrochloride

To a stirred solution of (R)-2-methyl-N—((S)-1-(6-phenoxypyridin-3-yl)ethyl)propane-2-sulfinamide (0.6 g, 1.8 mmol, 1.0 eq.) in methanol (10 mL) was added 4N HCl in dioxane (2 mL) at RT. The resulting mixture was stirred for 16 h. Following this, the reaction mixture was evaporated under reduced pressure to get solid. This solid washed with ether and evaporated to give title compound (0.4 g 99%). LCMS: 215.0 [M+1]⁺;

### Step-5: Synthesis of (S)-4-ethyl-3-(4-methyl-6-((S)-1-(6-phenoxypyridin-3-yl)ethylamino)-1,3,5-triazin-2-yl)oxazolidin-2-one

In a microwave vial charged with (S)-1-(6-phenoxypyridin-3-yl)ethanamine hydrochloride (0.12 g, 0.56 mmol, 1.0 eq.) and (S)-3-(4-chloro-6-methyl-1,3,5-triazin-2-yl)-4-ethyloxazolidin-2-one (0.13 g, 0.56 mmol, 1.0 eq.) in DMSO (5 mL) was added N, N-Diisopropylethylamine (0.2 mL, 1.12 mmol, 2.0 eq.) at RT. The resulting mixture was heated at 130° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound as white solid (0.023 g, 10%), UPLC-MS (Method 4): Rt 2.41, m/z 421.5 [M+1]⁺; ¹H NMR (400 MHz, DMSO-d₆) δ ppm 8.56 (d, J=7.89 Hz, 1H) 8.13 (br. s., 1H) 7.81-7.90 (m, 2H) 7.39 (t, J=7.89 Hz, 2H) 7.20 (d, J=7.02 Hz, 2H) 7.03-7.11 (m, 2H) 6.96-7.03 (m, 2H) 5.07-5.12 (m, 1H) 4.52 (br. s., 1H) 4.35-4.40 (m, 1H) 4.13 (d, J=6.58 Hz, 2H) 2.22-2.28 (m, 3H) 1.45 (d, J=7.45 Hz, 3H) 1.14-1.26 (m, 3H) 0.81 (d, J=7.02 Hz, 2H) 0.73 (t, J=7.24 Hz, 2H).

### Example-55: Synthesis of (S)-3-(4-((S)-1-(4-(2-bromo-4-fluorophenoxy)phenyl)ethylamino)-6-methyl-1,3,5-triazin-2-yl)-4-ethyloxazolidin-2-one (Compound 1.78) and (S)-3-(4-((R)-1-(4-(2-bromo-4-fluorophenoxy)phenyl)ethylamino)-6-methyl-1,3,5-triazin-2-yl)-4-ethyloxazolidin-2-one (Compound 1.79)

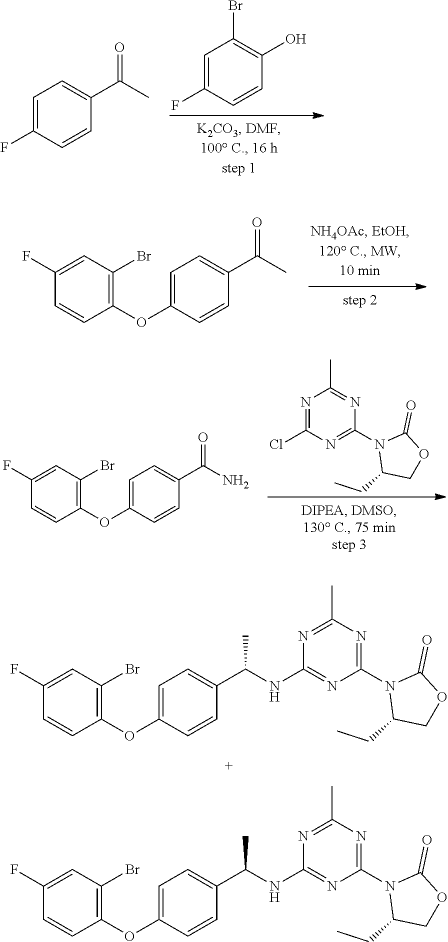

### Step-1: Synthesis of 1-(4-(2-bromo-4-fluorophenoxy)phenyl)ethanone

To a stirred solution of 1-(4-fluorophenyl)ethanone (1.5 g, 10.8 mmol, 1.0 eq.) and 2-bromo-4-fluorophenol (2.27 mL, 11.9 mmol, 1.1 eq.) in DMF (15 mL) was added K₂CO₃ (3.0 g, 21.7 mmol, 2.0 eq.) and heated at 100° C. for 16 h. Following this, reaction was allowed to cool to RT and filtered through celite pad, the celite pad washed with ethyl acetate and water. The aqueous layer was separated extracted using ethyl acetate (3×20 mL). The combined organic layers were washed with brine (30 mL), dried over anhydrous Na₂SO₄, filtered and concentrated under vacuum to get the solid residue which was purified by normal phase silica-gel column to get title compound (0.3 g, 9%). LCMS: 308.9 [M+1]⁺; ¹H NMR (400 MHz, CHLOROFORM-d) δ ppm 7.94-8.01 (m, 2H) 7.61 (dd, J=8.99, 5.92 Hz, 1H) 6.96-7.01 (m, 2H) 6.86 (ddd, J=8.88, 7.78, 2.63 Hz, 1H) 6.79 (dd, J=8.99, 2.85 Hz, 1H) 2.58 (s, 3H).

### Step-2: Synthesis of 1-(4-(2-bromo-4-fluorophenoxy)phenyl)ethanamine

In a microwave vial charged with 1-(4-(2-bromo-4-fluorophenoxy)phenyl)ethanone (0.3 g, 0.97 mmol, 1.0 eq.), Ammonium acetate (0.75 g, 9.7 mmol, 10.0 eq.) and sodium cyanoborohydride (0.06 g, 0.97 mmol, 1.0 eq.), in EtOH (10 mL). The resulting mixture was heated at 120° C. for 10 min. Following this, the reaction mixture was allowed to cool to RT, basified with 6N NaOH until pH˜10 and extracted with EtOAc (3×15 mL). The combined organic layers were washed with brine (25 mL), dried over Na₂SO₄ and concentrated under reduced pressure to get the title compound which was carried forward without any further purification (0.15 g 50%). LCMS: 310.1 [M+1]⁺

### Step-3: Synthesis of (S)-3-(4-((S)-1-(4-(2-bromo-4-fluorophenoxy)phenyl)ethylamino)-6-methyl-1,3,5-triazin-2-yl)-4-ethyloxazolidin-2-one and (S)-3-(4-((R)-1-(4-(2-bromo-4-fluorophenoxy)phenyl)ethylamino)-6-methyl-1,3,5-triazin-2-yl)-4-ethyloxazolidin-2-one

In a microwave vial charged with 1-(4-(2-bromo-4-fluorophenoxy)phenyl)ethanamine (0.15 g, 0.48 mmol, 1.0 eq.), (S)-3-(4-chloro-6-methyl-1,3,5-triazin-2-yl)-4-ethyloxazolidin-2-one (0.12 g, 0.48 mmol, 1.0 eq.) and N, N-Diisopropylethylamine (0.2 mL, 0.96 mmol, 2.0 eq.), in DMSO (5 mL). The resulting mixture was heated at 130° C. for 75 min. Following this, the reaction mixture was allowed to cool to RT concentrated under vacuum diluted with water (10 mL) and extracted using ethyl acetate (3×10 mL). The combined organic layers were washed with brine (10 mL), dried over anhydrous Na₂SO₄, filtered and evaporated under reduced pressure to get solid residue. The obtained solid was purified by normal phase silica-gel column chromatography followed by reverse phase column chromatography to obtain the title compounds. Compound 1.78 (0.019 g, 8%), UPLC-MS (Method 6): Rt 5.99, m/z 516.5 [M+1]⁺; ¹H NMR (400 MHz, DMSO-d₆) δ ppm 8.59 (d, J=7.89 Hz, 1H) 7.73-7.80 (m, 1H) 7.35-7.44 (m, 2H) 7.01 (td, J=8.44, 2.85 Hz, 1H) 6.84-6.97 (m, 3H) 6.79 (dd, J=9.65, 3.07 Hz, 1H) 5.07 (t, J=7.24 Hz, 1H) 4.49 (t, J=8.11 Hz, 1H) 4.32-4.41 (m, 1H) 4.09-4.16 (m, 1H) 2.24 (s, 3H) 1.76 (d, J=7.45 Hz, 1H) 1.36-1.54 (m, 5H) 0.81 (t, J=7.24 Hz, 1H) 0.69 (t, J=7.45 Hz, 3H) and Compound 1.79 (0.007 g, 4%), UPLC-MS (Method 6): Rt 6.12, m/z 516.5 [M+1]⁺; ¹H NMR (400 MHz, DMSO-d₆) δ ppm 8.53 (d, J=8.33 Hz, 1H) 8.39 (d, J=8.77 Hz, 1H) 7.77 (dd, J=8.99, 6.36 Hz, 1H) 7.42 (d, J=8.77 Hz, 2H) 6.86-7.08 (m, 5H) 5.04-5.11 (m, 1H) 4.32-4.42 (m, 2H) 4.14 (d, J=5.26 Hz, 1H) 2.19-2.28 (m, 3H) 1.85 (br. s., 1H) 1.77 (d, J=7.45 Hz, 2H) 1.44 (d, J=7.02 Hz, 3H) 0.84 (t, J=7.45 Hz, 3H).

### Example-56: Synthesis of (S)-4-ethyl-3-(4-methyl-6-((R)-1-(4-(2-(trifluoromethyl)pyridin-3-yloxy)phenyl)ethylamino)-1,3,5-triazin-2-yl)oxazolidin-2-one (Compound 1.80) and (S)-4-ethyl-3-(4-methyl-6-((S)-1-(4-(2-(trifluoromethyl)pyridin-3-yloxy)phenyl)ethylamino)-1,3,5-triazin-2-yl)oxazolidin-2-one (Compound 1.81)

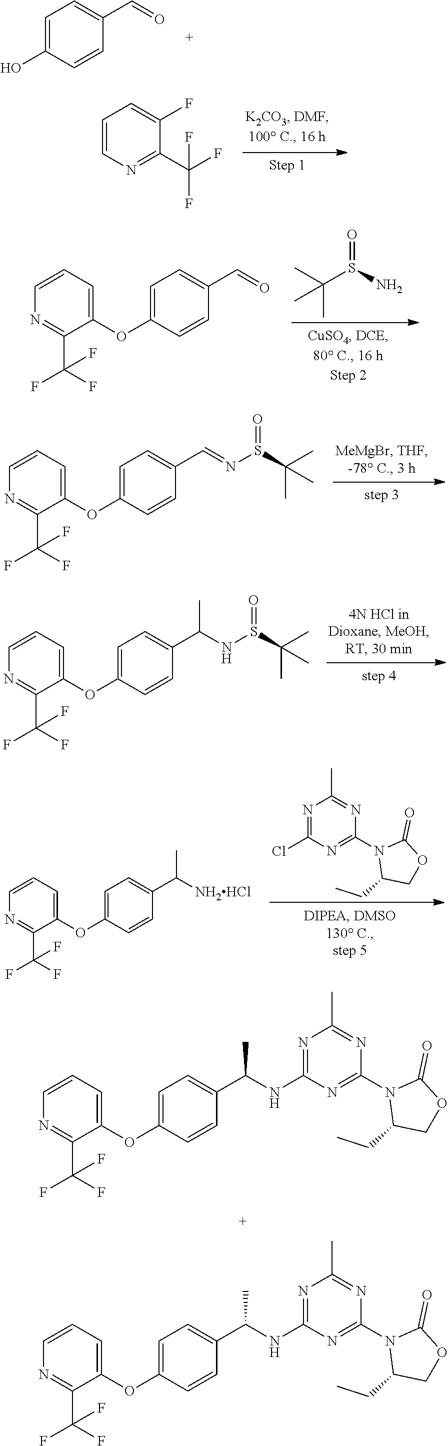

### Step-1: Synthesis of 4-(2-(trifluoromethyl)pyridin-3-yloxy)benzaldehyde

To a stirred solution of 3-fluoro-2-(trifluoromethyl)pyridine (1.0 g, 6.06 mmol, 1.0 eq.) and 4-hydroxybenzaldehyde (0.73 g, 6.06 mmol, 1.0 eq.) in DMF (20 mL) was added K₂CO₃ (2.5 g, 18.1 mmol, 3.0 eq.). The resulting mixture heated at 100° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound (0.9 g, 55%). LCMS: 268.1 [M+1]⁺; ¹H NMR (400 MHz, CHLOROFORM-d) δ ppm 9.98 (s, 1H) 8.56 (d, J=3.51 Hz, 1H) 7.92 (m, J=8.77 Hz, 2H) 7.54 (d, J=4.82 Hz, 1H) 7.46 (d, J=7.89 Hz, 1H) 7.12 (m, J=8.77 Hz, 2H).

### Step-2: Synthesis of (R,E)-2-methyl-N-(4-(2-(trifluoromethyl)pyridin-3-yloxy)benzylidene)propane-2-sulfinamide

To a stirred solution of 4-(2-(trifluoromethyl)pyridin-3-yloxy)benzaldehyde (0.9 g, 2.4 mmol, 1.0 eq.) and Copper(II) sulfate (1.1 g, 7.2 mmol, 3.0 eq.) in dichloroethane (10 mL) was added (R)-2-methylpropane-2-sulfinamide (0.58 g, 4.86 mmol, 2.0 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound (0.7 g, 78%). LCMS: 371.3 [M+1]⁺; ¹H NMR (400 MHz, DMSO-d₆) δ ppm 8.59 (t, J=2.85 Hz, 1H) 8.55 (s, 1H) 8.01 (m, J=8.77 Hz, 2H) 7.78-7.82 (m, 2H) 7.23 (m, J=8.33 Hz, 2H) 1.18 (s, 9H).

### Step-3: Synthesis of (R)-2-methyl-N-(1-(4-(2-(trifluoromethyl)pyridin-3-yloxy)phenyl)ethyl)propane-2-sulfinamide

To a stirred solution of (R,E)-2-methyl-N-(4-(2-(trifluoromethyl)pyridin-3-yloxy)benzylidene)propane-2-sulfinamide (0.7 g, 1.8 mmol, 1.0 eq.) in THF (10 mL) was added drop wise 3 molar methylmagnesium bromide (1.8 mL, 5.4 mmol, 3.0 eq.) at −78° C. The resulting mixture was stirred for 3 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.5 g 71%). LCMS: 387.3 [M+1]⁺;

### Step-4: Synthesis of 1-(4-(2-(trifluoromethyl)pyridin-3-yloxy)phenyl)ethanamine hydrochloride

To a stirred solution of (R)-2-methyl-N-(1-(4-(2-(trifluoromethyl)pyridin-3-yloxy)phenyl)ethyl)propane-2-sulfinamide (0.5 g, 1.2 mmol, 1.0 eq.) in methanol (10 mL) was added 4N HCl in dioxane (1 mL) at RT. The resulting mixture was stirred for 30 min. Following this, the reaction mixture was evaporated under reduced pressure to get solid. This solid washed with ether and evaporated to give title compound (0.25 g 69%). LCMS: 282.9 [M+1]⁺

### Step-5: Synthesis of (S)-4-ethyl-3-(4-methyl-6-((R)-1-(4-(2-(trifluoromethyl)pyridin-3-yloxy)phenyl)ethylamino)-1,3,5-triazin-2-yl)oxazolidin-2-one and (S)-4-ethyl-3-(4-methyl-6-((S)-1-(4-(2-(trifluoromethyl)pyridin-3-yloxy)phenyl)ethylamino)-1,3,5-triazin-2-yl)oxazolidin-2-one

In a microwave vial charged with 1-(4-(2-(trifluoromethyl)pyridin-3-yloxy)phenyl)ethanamine hydrochloride (0.2 g, 0.70 mmol, 1.0 eq.) and (S)-3-(4-chloro-6-methyl-1,3,5-triazin-2-yl)-4-ethyloxazolidin-2-one (0.17 g, 0.70 mmol, 1.0 eq.) in DMSO (3 mL) was added N, N-Diisopropylethylamine (0.24 mL, 1.4 mmol, 2.0 eq.) at RT. The resulting mixture was heated at 130° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds. Compound 1.80 (0.09 g, 3%), UPLC-MS (Method 6): Rt 5.33, m/z 489.5 [M+1]⁺; ¹H NMR (400 MHz, DMSO-d₆) δ ppm 8.54 (d, J=7.89 Hz, 1H) 8.39-8.51 (m, 2H) 7.70 (dd, J=8.77, 4.39 Hz, 1H) 7.51-7.58 (m, 1H) 7.46 (d, J=8.33 Hz, 2H) 7.03-7.10 (m, 2H) 5.25-5.33 (m, 1H) 5.05-5.16 (m, 1H) 4.56 (br. s., 1H) 4.34-4.44 (m, 2H) 4.12-4.18 (m, 1H) 2.21-2.28 (m, 3H) 1.72-1.90 (m, 3H) 1.45 (d, J=7.02 Hz, 3H) 0.80-0.89 (m, 3H) and Compound 1.81 (0.09 g 24%), UPLC-MS (Method 6): Rt 5.10, m/z 489.5 [M+1]⁺; ¹H NMR (400 MHz, DMSO-d₆) δ ppm 8.58 (d, J=7.89 Hz, 1H) 8.40-8.52 (m, 1H) 7.70 (dd, J=8.33, 4.39 Hz, 1H) 7.37-7.57 (m, 4H) 7.02-7.09 (m, 2H) 5.06-5.15 (m, 1H) 4.46-4.61 (m, 1H) 4.30-4.43 (m, 1H) 4.09-4.18 (m, 1H) 2.25 (s, 3H) 1.72-1.82 (m, 1H) 1.39-1.55 (m, 5H) 0.83 (t, J=7.24 Hz, 1H) 0.71 (t, J=7.45 Hz, 2H).

### Example-57: Synthesis of (S)-4-ethyl-3-(4-methyl-6-((S)-1-(6-phenoxybiphenyl-3-yl)ethylamino)-1,3,5-triazin-2-yl)oxazolidin-2-one (Compound 1.82)

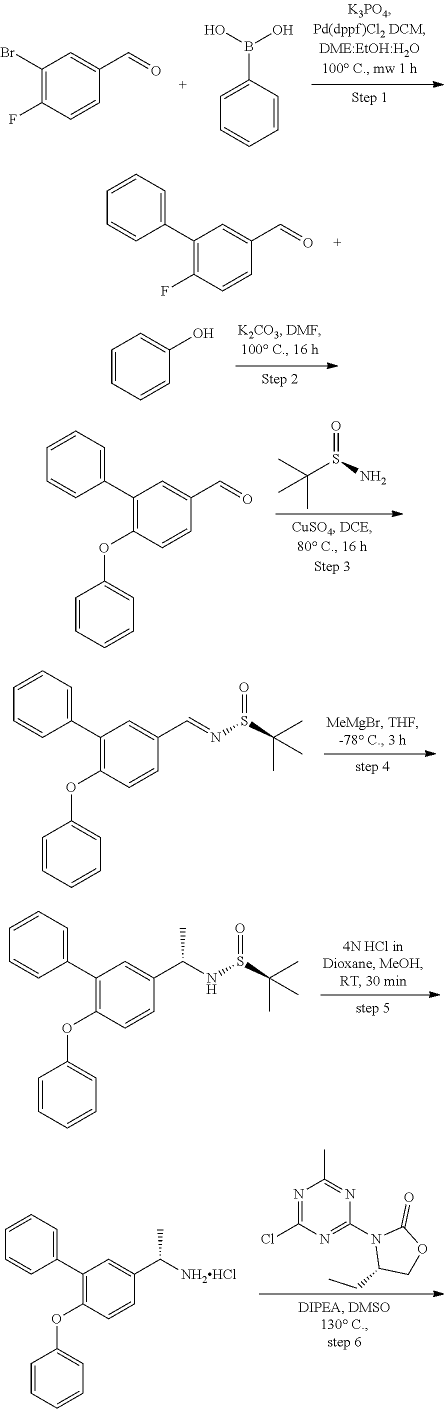

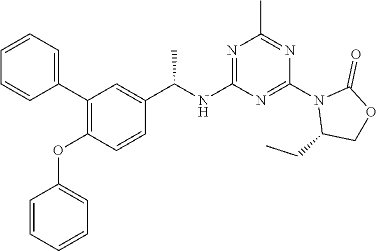 [SMILES: CC[C@H]1COC(=O)N1c1nc(C)nc(N[C@@H](C)c2ccc(Oc3ccccc3)c(-c3ccccc3)c2)n1]

### Step-1: Synthesis of 6-fluorobiphenyl-3-carbaldehyde

To a stirred solution of 3-bromo-4-fluorobenzaldehyde (1.0 g, 4.9 mmol, 1.0 eq.) and phenylboronic acid (0.66 g, 5.4 mmol, 1.1 eq.) in dimethoxyethane:H₂O:EtOH (7:3:2 mL) was added K₃PO₄ (2.07 g, 9.8 mmol, 2.0 eq.). The reaction mixture was purged with N₂ for about 15 min and Pd(dppf)Cl₂-DCM complex (0.39 g, 0.1 mol %) was added. Reaction mixture was re-purged with N₂ and heated at 100° C. for 1 h under microwave irradiation. Following this, reaction was allowed to cool to RT and filtered through celite pad, the celite pad washed with ethyl acetate and water. The aqueous layer was separated extracted using ethyl acetate (3×10 mL). The combined organic layers were washed with brine (20 mL), dried over anhydrous Na₂SO₄, filtered and concentrated under vacuum to get the solid residue which was purified by was purified by normal phase silica-gel column chromatography to get the title compound (0.9 g, 91%). ¹H NMR (400 MHz, CHLOROFORM-d) δ ppm 10.02 (s, 1H) 8.01 (dd, J=7.45, 1.75 Hz, 1H) 7.85-7.91 (m, 1H) 7.58 (d, J=7.45 Hz, 2H) 7.41-7.52 (m, 3H) 7.29-7.35 (m, 1H) 7.26 (s, 1H).

### Step-2: Synthesis of 6-phenoxybiphenyl-3-carbaldehyde

To a stirred solution of 6-fluorobiphenyl-3-carbaldehyde (0.9 g, 4.9 mmol, 1.0 eq.) and phenol (0.51 g, 5.44 mmol, 1.1 eq.) in DMF (20 mL) was added K₂CO₃ (1.3 g, 9.8 mmol, 2.0 eq.). The resulting mixture heated at 100° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound (0.9 g, 67%). LCMS: 275.0 [M+1]⁺; ¹H NMR (400 MHz, CHLOROFORM-d) δ ppm 9.98 (s, 1H) 7.98 (d, J=2.19 Hz, 1H) 7.78 (dd, J=8.33, 2.19 Hz, 1H) 7.59-7.64 (m, 1H) 7.41-7.45 (m, 1H) 7.34-7.40 (m, 2H) 7.14-7.25 (m, 2H) 7.04 (d, J=7.45 Hz, 2H) 6.99 (d, J=8.33 Hz, 1H) 6.83-6.92 (m, 2H).

### Step-3: Synthesis of (R,E)-2-methyl-N-((6-phenoxybiphenyl-3-yl)methylene)propane-2-sulfinamide

To a stirred solution of 6-phenoxybiphenyl-3-carbaldehyde (0.9 g, 3.2 mmol, 1.0 eq.) and Copper(II) sulfate (1.02 g, 6.5 mmol, 2.0 eq.) in dichloroethane (10 mL) was added (R)-2-methylpropane-2-sulfinamide (0.79 g, 6.5 mmol, 2.0 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound (0.7 g, 54%). LCMS: 378.1 [M+1]⁺; ¹H NMR (400 MHz, DMSO-d₆) δ ppm 8.59 (s, 1H) 8.04 (d, J=1.75 Hz, 1H) 7.93 (dd, J=8.55, 1.97 Hz, 1H) 7.61 (d, J=7.02 Hz, 2H) 7.37-7.47 (m, 5H) 7.18 (d, J=7.45 Hz, 1H) 7.02 (d, J=8.33 Hz, 1H) 7.07 (d, J=7.89 Hz, 2H) 1.17-1.20 (m, 9H).

### Step-4: Synthesis of (R)-2-methyl-N—((S)-1-(6-phenoxybiphenyl-3-yl)ethyl)propane-2-sulfinamide

To a stirred solution of (R,E)-2-methyl-N-((6-phenoxybiphenyl-3-yl)methylene)propane-2-sulfinamide (0.7 g, 1.8 mmol, 1.0 eq.) in THF (10 mL) was added drop wise 3 molar methylmagnesium bromide (1.8 mL, 5.5 mmol, 3.0 eq.) at −78° C. The resulting mixture was stirred for 3 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.32 g 44%). LCMS: 394.4 [M+1]⁺; ¹H NMR (400 MHz, DMSO-d₆) δ ppm 7.46-7.53 (m, 3H) 7.35-7.42 (m, 3H) 7.29-7.34 (m, 3H) 7.04 (t, J=7.24 Hz, 1H) 6.95 (d, J=8.33 Hz, 1H) 6.89 (d, J=7.89 Hz, 2H) 5.41 (d, J=5.70 Hz, 1H) 4.45-4.52 (m, 1H) 1.51 (d, J=7.02 Hz, 3H) 1.12 (s, 9H).

### Step-5: Synthesis of (S)-1-(6-phenoxybiphenyl-3-yl)ethanamine hydrochloride

To a stirred solution of (R)-2-methyl-N—((S)-1-(6-phenoxybiphenyl-3-yl)ethyl)propane-2-sulfinamide (0.32 g, 0.81 mmol, 1.0 eq.) in methanol (5 mL) was added 4N HCl in dioxane (2 mL) at RT. The resulting mixture was stirred for 30 min. Following this, the reaction mixture was evaporated under reduced pressure to get solid. This solid washed with ether and evaporated to give title compound (0.12 g 51%). LCMS: 290.1 [M+1]⁺

### Step-6: Synthesis of (S)-4-ethyl-3-(4-methyl-6-((S)-1-(6-phenoxybiphenyl-3-yl)ethylamino)-1,3,5-triazin-2-yl)oxazolidin-2-one

In a microwave vial charged with (S)-1-(6-phenoxybiphenyl-3-yl)ethanamine hydrochloride (0.12 g, 0.41 mmol, 1.0 eq.) and (S)-3-(4-chloro-6-methyl-1,3,5-triazin-2-yl)-4-ethyloxazolidin-2-one (0.11 g, 0.45 mmol, 1.1 eq.) in DMSO (3 mL) was added N, N-Diisopropylethylamine (0.15 mL, 0.82 mmol, 2.0 eq.) at RT. The resulting mixture was heated at 130° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds as white color solid (0.097 g, 47%), UPLC-MS (Method 6): Rt 6.42, m/z 496.6 [M+1]⁺; ¹H NMR (400 MHz, DMSO-d₆) δ ppm 8.52-8.67 (m, 1H) 8.46 (d, J=8.77 Hz, 1H) 7.45-7.57 (m, 3H) 7.24-7.43 (m, 7H) 6.99-7.06 (m, 1H) 6.95 (d, J=8.33 Hz, 1H) 6.81-6.91 (m, 2H) 5.12-5.21 (m, 1H) 4.48-4.60 (m, 1H) 4.34-4.42 (m, 1H) 4.09-4.19 (m, 1H) 2.24-2.30 (m, 3H) 1.46-1.54 (m, 4H) 0.83 (t, J=7.45 Hz, 1H) 0.67 (t, J=7.45 Hz, 2H).

### Example-58: Synthesis of (S)-4-ethyl-3-(4-methyl-6-((S)-1-(4-(p-tolyloxy)phenyl)ethylamino)-1,3,5-triazin-2-yl)oxazolidin-2-one (Compound 1.83)

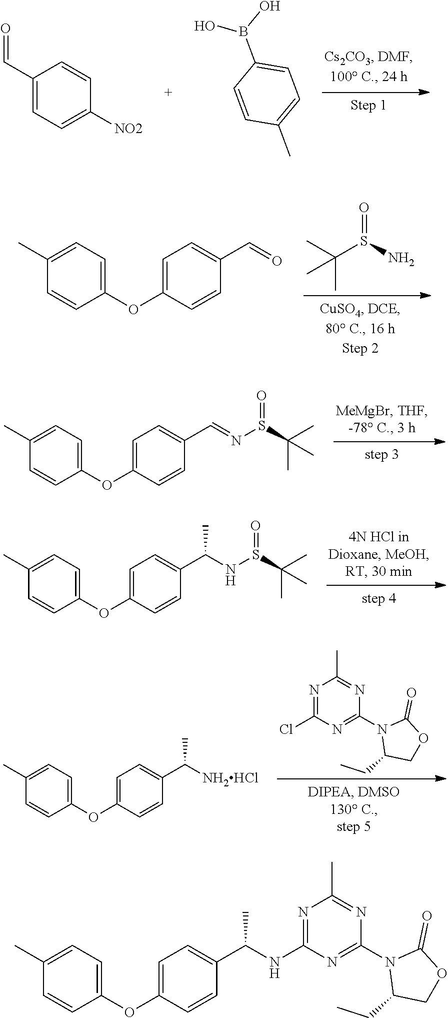

### Step-1: Synthesis of 4-(p-tolyloxy)benzaldehyde

To a stirred solution of 4-nitrobenzaldehyde (1.0 g, 6.6 mmol, 1.0 eq.), p-tolylboronic acid (0.45 g, 3.3 mmol, 0.5 eq.) in DMF (5 mL) was added Cs₂CO₃ (2.14 g, 6.6 mmol, 1.0 eq.) and Tris(triphenylphosphine)rhodium(I) chloride (0.061 g, 0.06 mmol, 0.01 eq.) at RT. The reaction mixture was stirred at same temperature for 30 min and heated at 100° C. for 24 h. Following this, reaction was allowed to cool to RT and added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by normal phase silica-gel column chromatography to get the title compound (0.2 g, 14%). LCMS: 212.9 [M+1]⁺; ¹H NMR (400 MHz, DMSO-d₆) δ ppm 9.91 (s, 1H) 7.87-7.94 (m, 2H) 7.28 (d, J=8.77 Hz, 2H) 7.05 (d, J=8.33 Hz, 2H) 7.08 (d, J=8.77 Hz, 2H) 2.33 (s, 3H).

### Step-2: Synthesis of (R,E)-2-methyl-N-(4-(p-tolyloxy)benzylidene)propane-2-sulfinamide

To a stirred solution of 4-(p-tolyloxy)benzaldehyde (0.2 g, 1.0 mmol, 1.0 eq.) and Copper(II) sulfate (0.38 g, 2.3 mmol, 2.5 eq.) in dichloroethane (10 mL) was added (R)-2-methylpropane-2-sulfinamide (0.2 g, 1.6 mmol, 1.8 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound (0.3 g, 90%). LCMS: 332.1 [M+1]⁺.

### Step-3: Synthesis of (R)-2-methyl-N-(1-(4-phenoxyphenyl)ethyl)propane-2-sulfinamide

To a stirred solution of (R,E)-2-methyl-N-(4-(p-tolyloxy)benzylidene)propane-2-sulfinamide (0.3 g, 1.0 mmol, 1.0 eq.) in DCM (10 mL) was added drop wise 3 molar methylmagnesium bromide (2.0 mL, 4.0 mmol, 4.0 eq.) at 0° C. The resulting mixture was stirred for 3 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.3 g 94%). LCMS: 316.1 [M+1]⁺

### Step-4: Synthesis of (S)-1-(4-(p-tolyloxy)phenyl)ethanamine hydrochloride

To a stirred solution of (R)-2-methyl-N-(1-(4-phenoxyphenyl)ethyl)propane-2-sulfinamide (0.3 g, 0.9 mmol, 1.0 eq.) in methanol (5 mL) was added 4N HCl in dioxane (1 mL) at RT. The resulting mixture was stirred for 3 h. Following this, the reaction mixture was evaporated under reduced pressure to get solid. This solid washed with ether and evaporated to give title compound (0.2 g 97%). LCMS: 228.0 [M+1]⁺

### Step-5: Synthesis of (S)-4-ethyl-3-(4-methyl-6-((S)-1-(4-(p-tolyloxy)phenyl)ethylamino)-1,3,5-triazin-2-yl)oxazolidin-2-one

In a microwave vial charged with (S)-1-(4-(p-tolyloxy)phenyl)ethanamine hydrochloride (0.2 g, 0.9 mmol, 1.0 eq.) and (S)-3-(4-chloro-6-methyl-1,3,5-triazin-2-yl)-4-ethyloxazolidin-2-one (0.21 g, 0.9 mmol, 1.0 eq.) in DMSO (5 mL) was added N, N-Diisopropylethylamine (0.4 mL, 1.8 mmol, 2.0 eq.) at RT. The resulting mixture was heated at 130° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds as white color solid (0.024 g, 7%), UPLC-MS (Method 4): Rt 2.73, m/z 434.6 [M+1]⁺; ¹H NMR (400 MHz, DMSO-d₆) δ ppm 8.53 (d, J=8.33 Hz, 1H) 8.37 (d, J=8.77 Hz, 1H) 7.30-7.40 (m, 1H) 7.16 (d, J=8.33 Hz, 1H) 6.78-6.97 (m, 3H) 6.60 (br. s., 1H) 5.24 (br. s., 1H) 5.02-5.16 (m, 1H) 4.50 (d, J=7.45 Hz, 1H) 4.31-4.45 (m, 1H) 4.09-4.19 (m, 1H) 3.79-3.95 (m, 1H) 3.65-3.79 (m, 1H) 2.26 (d, J=10.09 Hz, 4H) 1.70-1.84 (m, 1H) 1.51 (d, J=7.89 Hz, 1H) 1.37-1.47 (m, 3H) 1.21-1.29 (m, 2H) 0.77-0.88 (m, 1H) 0.72 (t, J=7.45 Hz, 2H).

### Example-59: Synthesis of (S)-4-ethyl-3-(4-((S)-1-(3-fluoro-4-phenoxyphenyl)ethylamino)-6-methyl-1,3,5-triazin-2-yl)oxazolidin-2-one (Compound 1.84)

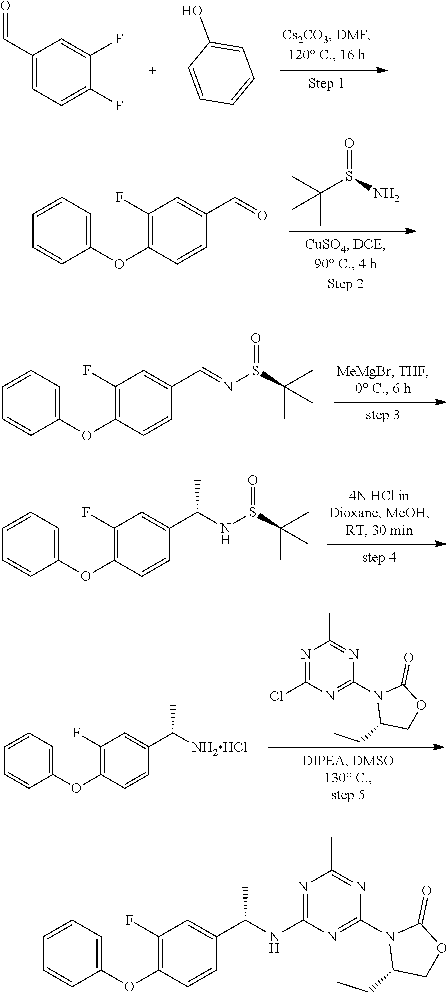

### Step-1: Synthesis of 4-(p-tolyloxy)benzaldehyde

To a stirred solution of 3,4-difluorobenzaldehyde (0.5 g, 5.31 mmol, 1.0 eq.), Phenol (0.95 g, 6.3 mmol, 1.2 eq.) in DMF (15 mL) was added Cs₂CO₃ (3.46 g, 10.6 mmol, 2.0 eq.) at RT. The reaction mixture was stirred at same temperature for 30 min and heated at 120° C. for 16 h. Following this, reaction was allowed to cool to RT and added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by normal phase silica-gel column chromatography to get the title compound as yellow solid (0.6 g, 52%). LCMS: 216.9 [M+1]⁺

### Step-2: Synthesis of (R,E)-N-(3-fluoro-4-phenoxybenzylidene)-2-methylpropane-2-sulfinamide

To a stirred solution of 4-(p-tolyloxy)benzaldehyde (0.6 g, 2.7 mmol, 1.0 eq.) and Copper(II) sulfate (1.32 g, 8.3 mmol, 3.0 eq.) in dichloroethane (10 mL) was added (R)-2-methylpropane-2-sulfinamide (0.67 g, 5.5 mmol, 2.0 eq.) at RT. The resulting mixture was heated at 90° C. for 4 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound (0.62 g, 70%). LCMS: 320.1 [M+1]⁺; ¹H NMR (400 MHz, CHLOROFORM-d) δ ppm 8.50 (d, J=1.32 Hz, 1H) 7.74 (dd, J=11.18, 1.97 Hz, 1H) 7.50 (d, J=8.33 Hz, 1H) 7.35-7.41 (m, 2H) 7.26 (s, 1H) 7.16-7.21 (m, 1H) 6.97-7.09 (m, 3H) 1.27 (s, 9H)

### Step-3: Synthesis of (R)—N—((S)-1-(3-fluoro-4-phenoxyphenyl)ethyl)-2-methylpropane-2-sulfinamide

To a stirred solution of (R,E)-N-(3-fluoro-4-phenoxybenzylidene)-2-methylpropane-2-sulfinamide (0.6 g, 1.8 mmol, 1.0 eq.) in DCM (20 mL) was added drop wise 3 molar methylmagnesium bromide (3.8 mL, 10.97 mmol, 6.0 eq.) at 0° C. The resulting mixture was stirred for 3 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.5 g 82%). LCMS: 336.3 [M+1]⁺; ¹H NMR (400 MHz, CHLOROFORM-d) δ ppm 7.29-7.37 (m, 2H) 6.91-7.21 (m, 6H) 4.56 (d, J=6.58 Hz, 1H) 1.51-1.56 (m, 3H) 1.19-1.24 (m, 9H).

### Step-4: Synthesis of (S)-1-(3-fluoro-4-phenoxyphenyl)ethanamine hydrochloride

To a stirred solution of (R)—N—((S)-1-(3-fluoro-4-phenoxyphenyl)ethyl)-2-methylpropane-2-sulfinamide (0.5 g, 1.49 mmol, 1.0 eq.) in methanol (10 mL) was added 4N HCl in dioxane (2 mL) at RT. The resulting mixture was stirred for 3 h. Following this, the reaction mixture was evaporated under reduced pressure to get solid. This solid washed with ether and evaporated to give title compound (0.32 g 92%). LCMS: 232.0 [M+1]⁺; ¹H NMR (400 MHz, DMSO-d₆) δ ppm 8.56 (br. s., 2H) 7.63 (dd, J=12.06, 1.97 Hz, 1H) 7.34-7.43 (m, 3H) 7.12-7.25 (m, 2H) 6.97 (d, J=7.89 Hz, 2H) 4.41-4.48 (m, 1H) 1.52 (d, J=6.58 Hz, 3H).

### Step-5: Synthesis of (S)-4-ethyl-3-(4-((S)-1-(3-fluoro-4-phenoxyphenyl)ethylamino)-6-methyl-1,3,5-triazin-2-yl)oxazolidin-2-one

In a microwave vial charged with (S)-1-(3-fluoro-4-phenoxyphenyl)ethanamine hydrochloride (0.2 g, 0.74 mmol, 1.0 eq.) and (S)-3-(4-chloro-6-methyl-1,3,5-triazin-2-yl)-4-ethyloxazolidin-2-one (0.18 g, 0.74 mmol, 1.0 eq.) in DMSO (2 mL) was added N, N-Diisopropylethylamine (0.4 mL, 2.2 mmol, 3.0 eq.) at RT. The resulting mixture was heated at 130° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds as white color solid (0.09 g, 28%), UPLC-MS (Method 6): Rt 5.67, m/z 438.5 [M+1]⁺; ¹H NMR (400 MHz, DMSO-d₆) δ ppm 8.59 (d, J=7.45 Hz, 1H) 8.42 (d, J=8.33 Hz, 1H) 7.32-7.44 (m, 3H) 7.07-7.25 (m, 4H) 6.88-6.97 (m, 2H) 5.28 (br. s., 1H) 5.09 (t, J=7.24 Hz, 1H) 4.49-4.61 (m, 1H) 4.35-4.44 (m, 1H) 4.10-4.19 (m, 1H) 2.26 (s, 3H) 1.78 (dd, J=14.03, 7.02 Hz, 1H) 1.39-1.54 (m, 5H) 0.83 (t, J=7.45 Hz, 1H) 0.72 (t, J=7.45 Hz, 3H).

### Example-60: Synthesis of (S)-4-ethyl-3-(4-methyl-6-((S)-1-(1-phenyl-1H-indol-6-yl)ethylamino)-1,3,5-triazin-2-yl)oxazolidin-2-one (Compound 1.85)

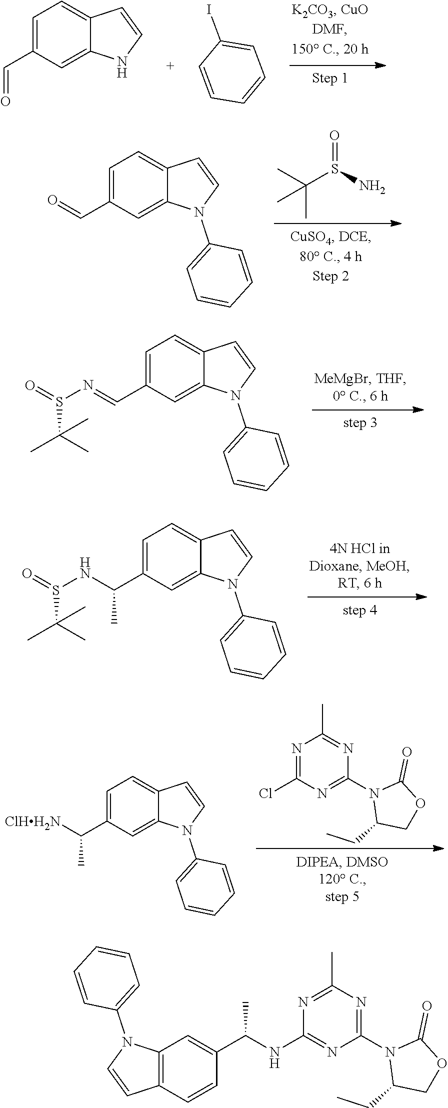

### Step-1: Synthesis of 1-phenyl-1H-indole-6-carbaldehyde

To a stirred solution of 1H-indole-5-carbaldehyde (2.0 g, 13.0 mmol, 1.0 eq.), iodobenzene (3.3 g, 16.0 mmol, 1.2 eq.) in DMF (20 mL) was added K₂CO₃ (2.6 g, 18.0 mmol, 1.4 eq.) and CuO (0.1 g, 1.3 mmol, 0.1 eq.) at RT. The reaction mixture was heated at 150° C. for 20 h. Following this, reaction was allowed to cool to RT and added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by normal phase silica-gel column chromatography to get the title compound as yellow solid (1.2 g, 41%). LCMS: 222.0 [M+1]⁺

### Step-2: Synthesis of (R,E)-2-methyl-N-((1-phenyl-1H-indol-6-yl)methylene)propane-2-sulfinamide

To a stirred solution of 1-phenyl-1H-indole-6-carbaldehyde (0.5 g, 2.2 mmol, 1.0 eq.) and Copper(II) sulfate (1.08 g, 6.7 mmol, 3.0 eq.) in dichloroethane (15 mL) was added (R)-2-methylpropane-2-sulfinamide (0.54 g, 4.5 mmol, 2.0 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound (0.7 g, 95%). LCMS: 325.2 [M+1]⁺; ¹H NMR (400 MHz, CHLOROFORM-d) δ ppm 8.69 (s, 1H) 8.15 (s, 1H) 7.80 (d, J=8.77 Hz, 1H) 7.46-7.62 (m, 5H) 7.37-7.44 (m, 2H) 6.79 (d, J=2.63 Hz, 1H) 1.28 (s, 9H).

### Step-3: Synthesis of (R)-2-methyl-N—((S)-1-(1-phenyl-1H-indol-6-yl)ethyl)propane-2-sulfinamide

To a stirred solution of (R,E)-2-methyl-N-((1-phenyl-1H-indol-6-yl)methylene)propane-2-sulfinamide (0.7 g, 2.1 mmol, 1.0 eq.) in DCM (20 mL) was added drop wise 3 molar methylmagnesium bromide (7.2 mL, 21.57 mmol, 10.0 eq.) at 0° C. The resulting mixture was stirred for 3 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.4 g 54%). LCMS: 341.3 [M+1]⁺

### Step-4: Synthesis of (S)-1-(1-phenyl-1H-indol-6-yl)ethanamine hydrochloride

To a stirred solution of (R)-2-methyl-N—((S)-1-(1-phenyl-1H-indol-6-yl)ethyl)propane-2-sulfinamide (0.4 g, 1.17 mmol, 1.0 eq.) in methanol (10 mL) was added 4N HCl in dioxane (3 mL) at RT. The resulting mixture was stirred for 6 h. Following this, the reaction mixture was evaporated under reduced pressure to get solid. This solid washed with ether and evaporated to give title compound (0.21 g 76%). LCMS: 237.1 [M+1]⁺

### Step-5: Synthesis of (S)-4-ethyl-3-(4-methyl-6-((S)-1-(1-phenyl-1H-indol-6-yl)ethylamino)-1,3,5-triazin-2-yl)oxazolidin-2-one

In a microwave vial charged with (S)-1-(1-phenyl-1H-indol-6-yl)ethanamine hydrochloride (0.2 g, 0.73 mmol, 1.0 eq.) and (S)-3-(4-chloro-6-methyl-1,3,5-triazin-2-yl)-4-ethyloxazolidin-2-one (0.21 g, 0.87 mmol, 1.2 eq.) in DMSO (2 mL) was added N, N-Diisopropylethylamine (0.4 mL, 2.2 mmol, 3.0 eq.) at RT. The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds as white color solid (0.09 g, 28%), UPLC-MS (Method 4): Rt 2.65, m/z 443.6 [M+1]⁺; ¹H NMR (400 MHz, DMSO-d₆) δ ppm 7.54-7.65 (m, 5H) 7.49 (d, J=7.89 Hz, 2H) 7.25 (m., 2H) 6.63 (d, J=3.07 Hz, 1H) 4.36 (s, 1H) 4.13 (br. s., 1H) 2.24 (s, 3H) 1.48-1.52 (m, 2H) 0.82 (t, J=7.67 Hz, 3H).

### Example-61: Synthesis of (S)-4-ethyl-3-(4-methyl-6-((S)-1-(3-methyl-4-phenoxyphenyl)ethylamino)-1,3,5-triazin-2-yl)oxazolidin-2-one (Compound 1.86)

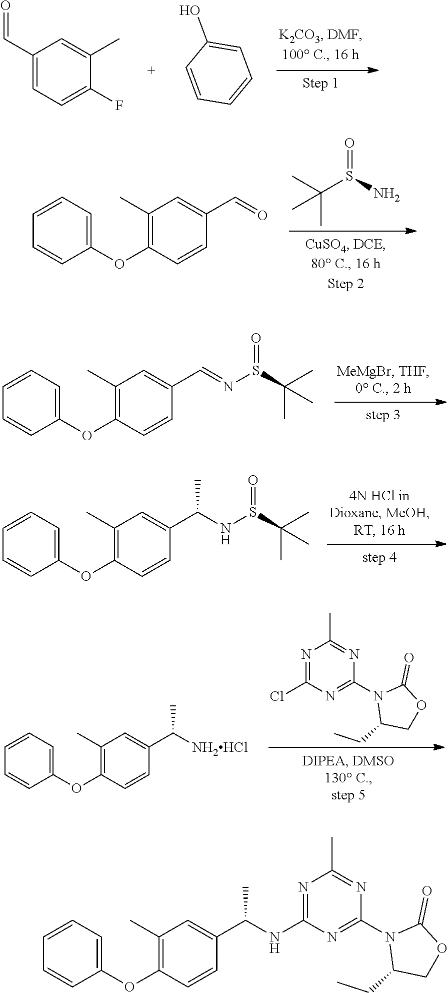

### Step-1: Synthesis of 3-methyl-4-phenoxybenzaldehyde

To a stirred solution of 4-fluoro-3-methylbenzaldehyde (1.0 g, 7.2 mmol, 1.0 eq.), Phenol (1.02 g, 10.0 mmol, 1.5 eq.) in DMF (10 mL) was added K₂CO₃ (2.5 g, 1.8 mmol, 2.5 eq.) at RT. The reaction mixture was heated at 100° C. for 16 h. Following this, reaction was allowed to cool to RT and added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by flash column chromatography to get the title compound (1.2 g, 78%). LCMS: 213.0 [M+1]⁺; ¹H NMR (400 MHz, DMSO-d₆) δ ppm 9.91 (s, 1H) 7.87 (s, 1H) 7.73 (dd, J=8.55, 1.97 Hz, 1H) 7.45 (t, J=7.89 Hz, 2H) 7.23 (t, J=7.24 Hz, 1H) 7.07 (d, J=7.89 Hz, 2H) 6.88 (d, J=8.77 Hz, 1H) 2.33 (s, 3H).

### Step-2: Synthesis of (R,E)-2-methyl-N-(3-methyl-4-phenoxybenzylidene)propane-2-sulfinamide

To a stirred solution of 3-methyl-4-phenoxybenzaldehyde (1.2 g, 5.0 mmol, 1.0 eq.) and Copper(II) sulfate (1.9 g, 12.0 mmol, 2.5 eq.) in dichloroethane (10 mL) was added (R)-2-methylpropane-2-sulfinamide (1.36 g, 11.0 mmol, 2.0 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by flash column chromatography to get the title compound (1.3 g, 82%). LCMS: 316.2 [M+1]⁺; ¹H NMR (400 MHz, DMSO-d₆) δ ppm 8.47 (s, 1H) 7.90 (s, 1H) 7.72-7.79 (m, 1H) 7.42 (t, J=7.89 Hz, 2H) 7.19 (t, J=7.45 Hz, 1H) 7.03 (d, J=7.89 Hz, 2H) 6.88 (d, J=8.33 Hz, 1H) 2.29 (s, 3H) 1.17 (s, 9H)

### Step-3: Synthesis of (R)-2-methyl-N—((S)-1-(3-methyl-4-phenoxyphenyl)ethyl)propane-2-sulfinamide

To a stirred solution of (R,E)-2-methyl-N-(3-methyl-4-phenoxybenzylidene)propane-2-sulfinamide (1.3 g, 4.0 mmol, 1.0 eq.) in DCM (10 mL) was added drop wise 3 molar methylmagnesium bromide (8.2 mL, 24.0 mmol, 6.0 eq.) at 0° C. The resulting mixture was stirred for 2 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by flash column chromatography to get the desired product (0.4 g 54%). LCMS: 332.2 [M+1]⁺; ¹H NMR (400 MHz, DMSO-d₆) δ ppm 7.35 (t, J=7.45 Hz, 1H) 7.28 (br. s., 1H) 7.12-7.25 (m, 2H) 7.00-7.11 (m, 2H) 6.74-6.92 (m, 2H) 5.31 (d, J=4.38 Hz, 1H) 4.30-4.41 (m, 1H) 2.14 (s, 3H) 1.46 (d, J=6.58 Hz, 3H) 1.11 (s, 9H).

### Step-4: Synthesis of (S)-1-(3-methyl-4-phenoxyphenyl)ethanamine hydrochloride

To a stirred solution of (R)-2-methyl-N—((S)-1-(3-methyl-4-phenoxyphenyl)ethyl)propane-2-sulfinamide (1.1 g, 3.3 mmol, 1.0 eq.) in methanol (5 mL) was added 4N HCl in dioxane (2 mL) at RT. The resulting mixture was stirred for 16 h. Following this, the reaction mixture was evaporated under reduced pressure to get solid. This solid washed with ether and evaporated to give title compound (0.6 g 80%). LCMS: 228.1 [M+1]⁺

### Step-5: Synthesis of (S)-4-ethyl-3-(4-methyl-6-((S)-1-(3-methyl-4-phenoxyphenyl)ethylamino)-1,3,5-triazin-2-yl)oxazolidin-2-one

In a microwave vial charged with (S)-1-(3-methyl-4-phenoxyphenyl)ethanamine hydrochloride (0.3 g, 1.3 mmol, 1.0 eq.) and (S)-3-(4-chloro-6-methyl-1,3,5-triazin-2-yl)-4-ethyloxazolidin-2-one (0.38 g, 1.58 mmol, 1.2 eq.) in DMSO (2 mL) was added N, N-Diisopropylethylamine (1.0 mL, 5.2 mmol, 4.0 eq.) at RT. The resulting mixture was heated at 130° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by flash column chromatography followed by reversed phase column chromatography to get the desired products as white color solid (0.13 g, 23%), UPLC-MS (Method 4): Rt 2.69, m/z 434.5 [M+1]⁺; ¹H NMR (400 MHz, DMSO-d₆) δ ppm 8.54 (d, J=7.89 Hz, 1H) 7.27-7.38 (m, 3H) 7.15-7.25 (m, 1H) 7.01-7.08 (m, 1H) 6.77-6.89 (m, 3H) 5.07 (d, J=7.02 Hz, 1H) 4.49-4.60 (m, 1H) 4.34-4.43 (m, 1H) 4.10-4.18 (m, 1H) 2.19-2.31 (m, 3H) 2.06-2.17 (m, 3H) 1.69-1.83 (m, 1H) 1.40-1.58 (m, 4H) 0.77-0.88 (m, 1H) 0.72 (t, J=7.45 Hz, 2H).

### Example-62: Synthesis of (S)-3-(4-((S)-1-(6-(4-chlorophenoxy)-2-methoxypyridin-3-yl)ethylamino)-6-methyl-1,3,5-triazin-2-yl)-4-ethyloxazolidin-2-one (Compound 1.87)

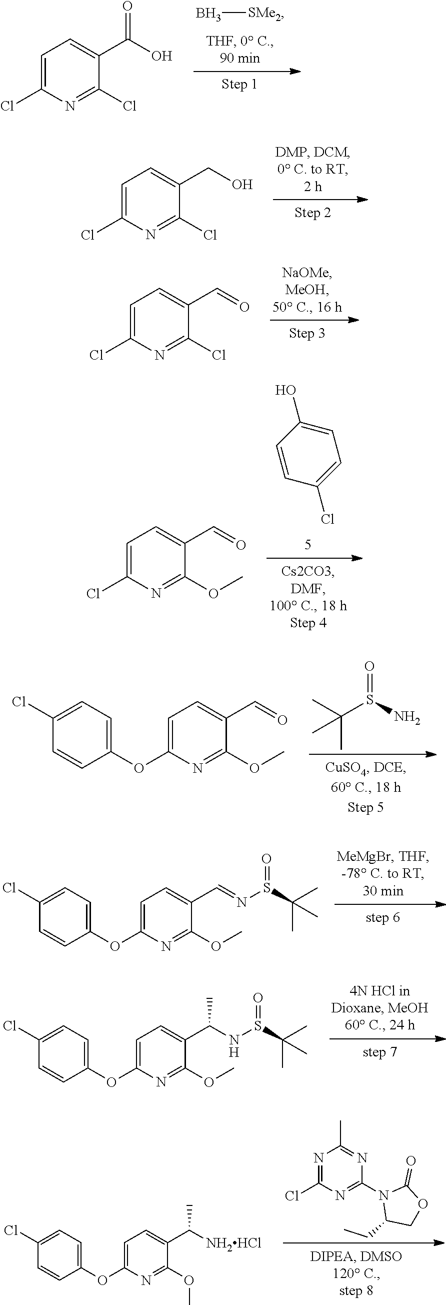

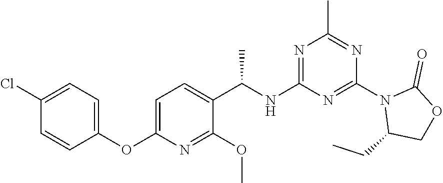 [SMILES: CC[C@H]1COC(=O)N1c1nc(C)nc(N[C@@H](C)c2ccc(Oc3ccc(Cl)cc3)nc2OC)n1]

### Step-1: Synthesis of (2,6-dichloropyridin-3-yl)methanol

To a stirred solution of 2,6-dichloronicotinic acid (3.0 g, 15.7 mmol, 1.0 eq.) in THF (20 mL) was added borane dimethylsulfide (2.3 g, 31.4 mmol, 2.0 eq.) at 0° C. The reaction mixture was stirred at same temperature for 2 h. Following this, the reaction quenched with saturated ammonium chloride (20 mL) extracted using ethyl acetate (3×20 mL). The combined organic layers were washed with brine (50 mL), dried over anhydrous Na₂SO₄, filtered and concentrated under vacuum to get desired product (2.5 g, 89%). LCMS: 178.1 [M+1]⁺

### Step-2: Synthesis of 2,6-dichloronicotinaldehyde

To a stirred solution of (2,6-dichloropyridin-3-yl)methanol (3.0 g, 16.8 mmol, 1.0 eq.) in DCM (50 mL) was added Dess-Martin periodinane (8.5 g, 20.2 mmol, 1.2 eq.) at RT. The reaction mixture was stirred at same temperature for 2 h. Following this, the reaction quenched with saturated 20% solution of sodium thiosulpate (20 mL) and extracted using DCM (3×20 mL). The combined organic layers were washed with brine (50 mL), dried over anhydrous Na₂SO₄, filtered and concentrated under vacuum to get desired product (2.15 g, 72%). ¹H NMR (400 MHz, CHLOROFORM-d) δ ppm 10.39 (s, 1H) 8.19 (d, J=8.33 Hz, 1H) 7.44 (d, J=7.89 Hz, 1H).

### Step-3: Synthesis of 6-chloro-2-methoxynicotinaldehyde

To a stirred solution of 2,6-dichloronicotinaldehyde (2.1 g, 11.93 mmol, 1.0 eq.) in MeOH (20 mL) was added NaOMe (0.65 g, 11.9 mmol, 1.0 eq.) at RT. The resulting mixture was heated to 50° C. and stirred for 16 h. Following this, the reaction mixture was evaporated under reduced pressure to get crude product. The crude purified by flash column to get title compound (0.6 g 29%). LCMS: 172.2 [M+1]⁺

### Step-4: Synthesis of 6-(4-chlorophenoxy)-2-methoxynicotinaldehyde

To a stirred solution of 6-chloro-2-methoxynicotinaldehyde (0.5 g, 2.9 mmol, 1.0 eq.), 4-chlorophenol (0.75 g, 5.8 mmol, 2.0 eq.) in DMF (10 mL) was added Cs₂CO₃ (1.89 g, 5.8 mmol, 2.0 eq.) at RT. The reaction mixture was heated at 100° C. for 18 h. Following this, reaction was allowed to cool to RT and added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by flash column chromatography to get the desired product (0.25 g, 32%). LCMS: 263.9 [M+1]⁺;

### Step-5: Synthesis of (R,E)-N-((6-(4-chlorophenoxy)-2-methoxypyridin-3-yl)methylene)-2-methylpropane-2-sulfinamide

To a stirred solution of 6-(4-chlorophenoxy)-2-methoxynicotinaldehyde (0.25 g, 0.91 mmol, 1.0 eq.) and Copper(II) sulfate (0.22 g, 1.8 mmol, 2.0 eq.) in dichloroethane (10 mL) was added (R)-2-methylpropane-2-sulfinamide (1.36 g, 11.0 mmol, 2.0 eq.) at RT. The resulting mixture was heated at 60° C. for 18 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by flash column chromatography to get the desired product (0.25 g, 75%). LCMS: 316.2 [M+1]⁺;

### Step-6: Synthesis of (R)—N—((S)-1-(6-(4-chlorophenoxy)-2-methoxypyridin-3-yl)ethyl)-2-methylpropane-2-sulfinamide

To a stirred solution of (R,E)-N-((6-(4-chlorophenoxy)-2-methoxypyridin-3-yl)methylene)-2-methylpropane-2-sulfinamide (0.25 g, 0.68 mmol, 1.0 eq.) in THF (5 mL) was added drop wise 3 M methylmagnesium bromide (1.2 mL, 3.4 mmol, 5.0 eq.) at −78° C. The resulting mixture was stirred for 30 at same temperature allowed to RT and stirred for another 30 min. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by flash column chromatography to get the desired product (0.2 g 70%). LCMS: 383.4 [M+1]⁺;

### Step-7: Synthesis of (S)-1-(6-(4-chlorophenoxy)-2-methoxypyridin-3-yl)ethanamine hydrochloride

To a stirred solution of (R)—N—((S)-1-(6-(4-chlorophenoxy)-2-methoxypyridin-3-yl)ethyl)-2-methylpropane-2-sulfinamide (0.2 g, 0.52 mmol, 1.0 eq.) in methanol (5 mL) was added 4N HCl in dioxane (1 mL) at RT. The resulting mixture was heated to 60° C. and stirred for 16 h. Following this, the reaction mixture was evaporated under reduced pressure to get solid. This solid washed with ether and evaporated to give title compound (0.1 g 69%). LCMS: 280.0 [M+1]⁺

### Step-8: Synthesis of (S)-3-(4-((S)-1-(6-(4-chlorophenoxy)-2-methoxypyridin-3-yl)ethylamino)-6-methyl-1,3,5-triazin-2-yl)-4-ethyloxazolidin-2-one

In a microwave vial charged with (S)-1-(6-(4-chlorophenoxy)-2-methoxypyridin-3-yl)ethanamine hydrochloride (0.05 g, 0.13 mmol, 1.0 eq.) and (S)-3-(4-chloro-6-methyl-1,3,5-triazin-2-yl)-4-ethyloxazolidin-2-one (0.031 g, 0.13 mmol, 1.0 eq.) in DMSO (2 mL) was added N, N-Diisopropylethylamine (0.1 mL, 0.78 mmol, 6.0 eq.) at RT. The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by flash column chromatography followed by reversed phase column chromatography to get the desired products as white color solid (0.003 g, 5%), UPLC-MS (Method 7): Rt 3.98, m/z 485.5 [M+1]⁺; ¹H NMR (400 MHz, DMSO-d₆) δ ppm 8.58 (d, J=7.89 Hz, 1H) 7.76-7.87 (m, 1H) 7.43 (d, J=8.33 Hz, 2H) 7.13-7.22 (m, 2H) 6.54-6.62 (m, 1H) 5.49 (d, J=7.89 Hz, 1H) 5.40 (d, J=7.45 Hz, 1H) 4.29-4.43 (m, 2H) 4.05-4.17 (m, 2H) 3.67-3.75 (m, 1H) 3.55-3.60 (m, 3H) 2.22-2.28 (m, 3H) 1.44 (d, J=6.58 Hz, 2H) 0.83 (t, J=7.02 Hz, 1H) 0.73 (t, J=7.45 Hz, 2H).

### Example-63: Synthesis of (S)-4-ethyl-3-(4-methyl-6-((R)-1-(4-(phenylamino)phenyl)ethylamino)-1,3,5-triazin-2-yl)oxazolidin-2-one (Compound No. 1.88) and (S)-4-ethyl-3-(4-methyl-6-((S)-1-(4-(phenylamino)phenyl)ethylamino)-1,3,5-triazin-2-yl)oxazolidin-2-one (Compound No. 1.89)

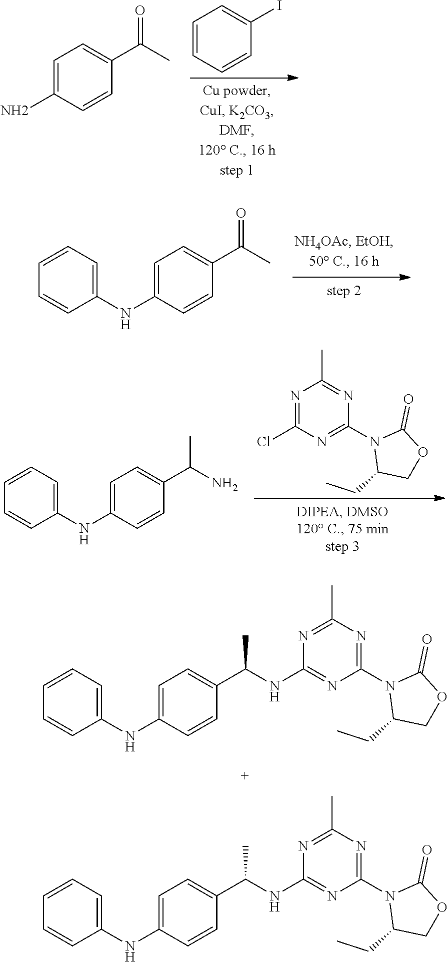

### Step-1: Synthesis of 1-(4-(phenylamino)phenyl)ethanone

To a stirred solution of 1-(4-aminophenyl)ethanone (1.2 g, 5.88 mmol, 1.0 eq.) and iodobenzene (0.79 g, 5.88 mmol, 1.0 eq.) in DMF (15 mL) was added K₂CO₃ (2.43 g, 17.64 mmol, 3.0 eq.), Cu powder (0.1 g) and CuI (0.011 g, 0.058 mmol, 0.01 eq.) at RT. The resulting mixture heated at 120° C. for 16 h. Following this, reaction was allowed to cool to RT and filtered through celite pad, the celite pad washed with ethyl acetate and water. The aqueous layer was separated extracted using ethyl acetate (3×20 mL). The combined organic layers were washed with brine (30 mL), dried over anhydrous Na₂SO₄, filtered and concentrated under vacuum to get the solid residue which purified by flash column to get title compound (0.3 g, 24%). LCMS: 212.1 [M+1]⁺; ¹H NMR (400 MHz, CHLOROFORM-d) δ ppm 7.87 (m, J=8.77 Hz, 2H) 7.34 (d, J=7.89 Hz, 2H) 7.19 (d, J=7.45 Hz, 2H) 6.99 (m, J=8.77 Hz, 2H) 2.53 (s, 3H).

### Step-2: Synthesis of 4-(1-aminoethyl)-N-phenylaniline

To a stirred solution of 1-(4-(phenylamino)phenyl)ethanone (0.2 g, 0.94 mmol, 1.0 eq.) and Ammonium acetate (0.72 g, 9.4 mmol, 10.0 eq.) in EtOH (10 mL) was added sodium cyanoborohydride (0.05 g, 0.94 mmol, 1.0 eq.) at RT. The resulting mixture was heated at 50° C. for 16 h. Following this, the reaction mixture was allowed to cool to RT, basified with 6N NaOH until pH˜10 and extracted with EtOAc (3×15 mL). The combined organic layers were washed with brine (25 mL), dried over Na₂SO₄ and concentrated under reduced pressure to get the desired product which was carried forward without any further purification (0.18 g 90%). LCMS: 213.3 [M+1]⁺

### Step-3: Synthesis of (S)-4-ethyl-3-(4-methyl-6-((R)-1-(4-(phenylamino)phenyl)ethylamino)-1,3,5-triazin-2-yl)oxazolidin-2-one and (S)-4-ethyl-3-(4-methyl-6-((S)-1-(4-(phenylamino)phenyl)ethylamino)-1,3,5-triazin-2-yl)oxazolidin-2-one

In a microwave vial charged with 4-(1-aminoethyl)-N-phenylaniline (0.18 g, 0.84 mmol, 1.0 eq.), (S)-3-(4-chloro-6-methyl-1,3,5-triazin-2-yl)-4-ethyloxazolidin-2-one (0.2 g, 0.84 mmol, 1.0 eq.) and N, N-Diisopropylethylamine (0.44 mL, 2.54 mmol, 3.0 eq.), in DMSO (2 mL). The resulting mixture was heated at 120° C. for 75 min. Following this, the reaction mixture was allowed to cool to RT diluted with water (10 mL) and extracted using ethyl acetate (3×10 mL). The combined organic layers were washed with brine (10 mL), dried over anhydrous Na₂SO₄, filtered and evaporated under reduced pressure to get solid residue. The obtained solid was purified by reverse phase column chromatography to get title compounds. Compound 1.88 (0.005 g, 14%), UPLC-MS (Method 6): Rt 5.40, m/z 419.5 [M+1]⁺; ¹H NMR (400 MHz, DMSO-d₆) δ ppm 8.43 (d, J=8.33 Hz, 1H) 8.07 (s, 1H) 7.16-7.29 (m, 4H) 7.01 (t, J=8.33 Hz, 4H) 6.78 (t, J=7.24 Hz, 1H) 5.11-5.24 (m, 1H) 5.03 (d, J=7.45 Hz, 1H) 4.56 (br. s., 1H) 4.40 (dt, J=15.68, 7.73 Hz, 2H) 4.15 (dd, J=8.33, 2.63 Hz, 1H) 2.18-2.29 (m, 3H) 1.74-1.90 (m, 2H) 1.42 (d, J=7.02 Hz, 3H) 0.79-0.89 (m, 3H) and Compound 1.89 (0.004 g, 13%), UPLC-MS (Method 6): Rt 5.45, m/z 419.5 [M+1]⁺; ¹H NMR (400 MHz, DMSO-d₆) δ ppm 8.47 (d, J=8.33 Hz, 1H) 8.04-8.10 (m, 1H) 7.13-7.30 (m, 4H) 6.93-7.07 (m, 4H) 6.77 (t, J=7.24 Hz, 1H) 5.20 (d, J=7.02 Hz, 1H) 5.04 (d, J=7.45 Hz, 1H) 4.53 (br. s., 1H) 4.35-4.43 (m, 1H) 4.13 (d, J=8.33 Hz, 1H) 2.17-2.30 (m, 3H) 1.50 (dd, J=14.47, 7.45 Hz, 1H) 1.41 (d, J=6.58 Hz, 2H) 0.71-0.88 (m, 3H).

### Example-64: Synthesis of (S)-4-ethyl-3-(4-methyl-6-((S)-1-(1-phenyl-1H-indol-5-yl)ethylamino)-1,3,5-triazin-2-yl)oxazolidin-2-one (Compound 1.90)

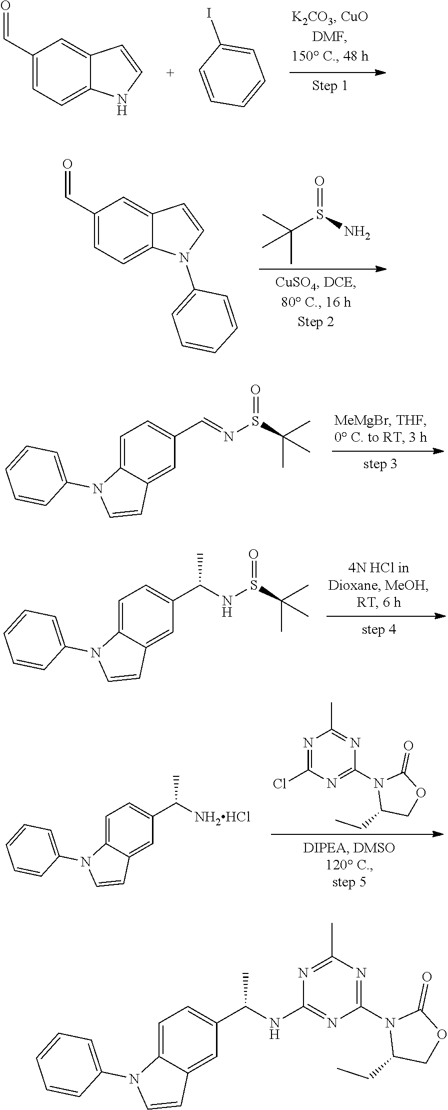

### Step-1: Synthesis of 1-phenyl-1H-indole-5-carbaldehyde

To a stirred solution of 1H-indole-5-carbaldehyde (2.0 g, 13.0 mmol, 1.0 eq.), iodobenzene (3.3 g, 16.0 mmol, 1.2 eq.) in DMF (20 mL) was added K₂CO₃ (2.6 g, 18.0 mmol, 1.4 eq.) and CuO (0.1 g, 1.3 mmol, 0.1 eq.) at RT. The reaction mixture was heated at 150° C. for 48 h. Following this, reaction was allowed to cool to RT and added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by flash column chromatography to get the desired product as yellow solid (1.2 g, 41%). LCMS: 222.0 [M+1]⁺

### Step-2: Synthesis of (R,E)-2-methyl-N-((1-phenyl-1H-indol-5-yl)methylene)propane-2-sulfinamide

To a stirred solution of 1-phenyl-1H-indole-5-carbaldehyde (0.9 g, 4.07 mmol, 1.0 eq.) and Copper(II) sulfate (1.6 g, 10.7 mmol, 2.5 eq.) in dichloroethane (20 mL) was added (R)-2-methylpropane-2-sulfinamide (1.03 g, 8.55 mmol, 2.1 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the desired product (1.2 g, 91%). LCMS: 325.2 [M+1]⁺; ¹H NMR (400 MHz, DMSO-d₆) δ ppm 8.61 (s, 1H) 8.26 (s, 1H) 7.77-7.84 (m, 2H) 7.56-7.69 (m, 5H) 7.47 (br. s., 1H) 6.87 (d, J=3.07 Hz, 1H) 1.19 (s, 9H).

### Step-3: Synthesis of (R)-2-methyl-N—((S)-1-(1-phenyl-1H-indol-5-yl)ethyl)propane-2-sulfinamide

To a stirred solution of (R,E)-2-methyl-N-((1-phenyl-1H-indol-5-yl)methylene)propane-2-sulfinamide (1.2 g, 3.7 mmol, 1.0 eq.) in DCM (20 mL) was added drop wise 3 molar methylmagnesium bromide (9.8 mL, 27.0 mmol, 8.0 eq.) at 0° C. The resulting mixture was allowed to RT and stirred for 3 h. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the desired product (0.8 g 63%). LCMS: 341.3 [M+1]⁺

### Step-4: Synthesis of (S)-1-(1-phenyl-1H-indol-5-yl)ethanamine hydrochloride

To a stirred solution of (R)-2-methyl-N—((S)-1-(1-phenyl-1H-indol-5-yl)ethyl)propane-2-sulfinamide (0.4 g, 1.17 mmol, 1.0 eq.) in methanol (10 mL) was added 4N HCl in dioxane (3 mL) at RT. The resulting mixture was stirred for 6 h. Following this, the reaction mixture was evaporated under reduced pressure to get solid. This solid washed with ether and evaporated to give title compound (0.21 g 76%). LCMS: 237.1 [M+1]⁺

### Step-5: Synthesis of (S)-4-ethyl-3-(4-methyl-6-((S)-1-(1-phenyl-1H-indol-5-yl)ethylamino)-1,3,5-triazin-2-yl)oxazolidin-2-one

In a microwave vial charged with (S)-1-(1-phenyl-1H-indol-5-yl)ethanamine hydrochloride (0.37 g, 1.63 mmol, 1.0 eq.) and (S)-3-(4-chloro-6-methyl-1,3,5-triazin-2-yl)-4-ethyloxazolidin-2-one (0.45 g, 1.87 mmol, 1.1 eq.) in DMSO (2 mL) was added N, N-Diisopropylethylamine (1.1 mL, 6.5 mmol, 4.0 eq.) at RT. The resulting mixture was heated at 120° C. for 90 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the desired products as white color solid (0.12 g, 16%), UPLC-MS (Method 7): Rt 3.83, m/z 443.5 [M+1]⁺; ¹H NMR (400 MHz, DMSO-d₆) δ ppm 8.58 (d, J=7.89 Hz, 1H) 7.55-7.66 (m, 5H) 7.48-7.52 (m, 1H) 7.34-7.42 (m, 1H) 7.21-7.29 (m, 1H) 6.60-6.69 (m, 1H) 5.28-5.37 (m, 1H) 5.19-5.28 (m, 1H) 4.52 (t, J=8.11 Hz, 1H) 4.33-4.43 (m, 1H) 4.10-4.17 (m, 1H) 2.18-2.29 (m, 3H) 1.72-1.83 (m, 1H) 1.38-1.61 (m, 4H) 0.82 (t, J=7.45 Hz, 1H) 0.70 (t, J=7.45 Hz, 2H).

### Example-65: Synthesis of (S)-4-ethyl-3-(4-((R)-1-(4-(4-fluorophenylthio)phenyl)ethylamino)-6-methyl-1,3,5-triazin-2-yl)oxazolidin-2-one (Compound 1.91) and (S)-4-ethyl-3-(4-((S)-1-(4-(4-fluorophenylthio)phenyl)ethylamino)-6-methyl-1,3,5-triazin-2-yl)oxazolidin-2-one (Compound 1.92)

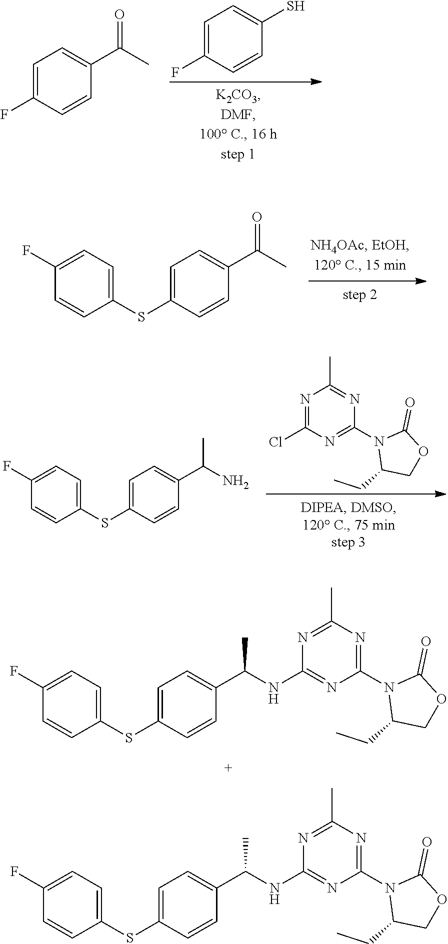

### Step-1: Synthesis of 1-(4-(4-fluorophenylthio)phenyl)ethanone

To a stirred solution of 1-(4-fluorophenyl)ethanone (1.0 g, 7.2 mmol, 1.0 eq.) and 4-fluorobenzenethiol (1.02 g, 7.9 mmol, 1.1 eq.) in DMF (10 mL) was added K₂CO₃ (2.0 g, 14.5 mmol, 2.0 eq.) at RT. The resulting mixture heated at 100° C. for 16 h. Following this, reaction was allowed to cool to RT and filtered through celite pad, the celite pad washed with ethyl acetate and water. The aqueous layer was separated extracted using ethyl acetate (3×20 mL). The combined organic layers were washed with brine (30 mL), dried over anhydrous Na₂SO₄, filtered and concentrated under vacuum to get the solid residue which purified by normal phase silica-gel column chromatography to get title compound (0.7 g, 39%). LCMS: 246.9 [M+1]⁺

### Step-2: Synthesis of 1-(4-(4-fluorophenylthio)phenyl)ethanamine

To a stirred solution of 1-(4-(4-fluorophenylthio)phenyl)ethanone (0.7 g, 3.0 mmol, 1.0 eq.) and Ammonium acetate (2.20 g, 30.0 mmol, 10.0 eq.) in EtOH (10 mL) was added sodium cyanoborohydride (0.19 g, 3.0 mmol, 1.0 eq.) at RT. The resulting mixture was heated at 120° C. for 15 min in microwave. Following this, the reaction mixture was allowed to cool to RT, basified with 6N NaOH until pH˜10 and extracted with EtOAc (3×15 mL). The combined organic layers were washed with brine (25 mL), dried over Na₂SO₄ and concentrated under reduced pressure to get the desired product which was carried forward without any further purification (0.2 g 26%). LCMS: 248.0 [M+1]⁺; ¹H NMR (400 MHz, DMSO-d₆) δ ppm 7.33-7.43 (m, 4H) 7.19-7.29 (m, 4H) 4.00 (br. s., 1H) 1.24 (d, J=6.14 Hz, 3H)

### Step-3: Synthesis of (S)-4-ethyl-3-(4-((R)-1-(4-(4-fluorophenylthio)phenyl)ethylamino)-6-methyl-1,3,5-triazin-2-yl)oxazolidin-2-one and (S)-4-ethyl-3-(4-((S)-1-(4-(4-fluorophenylthio)phenyl)ethylamino)-6-methyl-1,3,5-triazin-2-yl)oxazolidin-2-one

In a microwave vial charged with 1-(4-(4-fluorophenylthio)phenyl)ethanamine (0.2 g, 0.8 mmol, 1.0 eq.), (S)-3-(4-chloro-6-methyl-1,3,5-triazin-2-yl)-4-ethyloxazolidin-2-one (0.19 g, 0.8 mmol, 1.0 eq.) and N, N-Diisopropylethylamine (0.3 mL, 1.6 mmol, 2.0 eq.), in DMSO (5 mL). The resulting mixture was heated at 120° C. for 75 min. Following this, the reaction mixture was allowed to cool to RT diluted with water (10 mL) and extracted using ethyl acetate (3×10 mL). The combined organic layers were washed with brine (10 mL), dried over anhydrous Na₂SO₄, filtered and evaporated under reduced pressure to get solid residue. The obtained solid was purified reverse phase column chromatography to get title compounds. Compound 1.91 (0.01 g, 3%), UPLC-MS (Method 4): Rt 2.79, m/z 454.2 [M+1]⁺; ¹H NMR (400 MHz, DMSO-d₆) δ ppm 8.56 (d, J=8.33 Hz, 1H) 7.33-7.43 (m, 4H) 7.18-7.28 (m, 4H) 5.14-5.26 (m, 1H) 5.05 (d, J=7.45 Hz, 1H) 4.30-4.47 (m, 2H) 4.12-4.18 (m, 1H) 3.51 (br. s., 1H) 2.24 (s, 3H) 1.73-1.87 (m, 2H) 1.42 (d, J=7.02 Hz, 3H) 0.83 (t, J=7.45 Hz, 3H) and Compound 1.92 (0.03 g, 8%), UPLC-MS (Method 4): Rt 2.78, m/z 454.5 [M+1]⁺; ¹H NMR (400 MHz, DMSO-d₆) δ ppm 8.56 (d, J=7.45 Hz, 1H) 7.31-7.43 (m, 4H) 7.18-7.28 (m, 4H) 5.03-5.13 (m, 1H) 4.31-4.52 (m, 2H) 4.08-4.16 (m, 1H) 2.12-2.30 (m, 3H) 1.32-1.48 (m, 4H) 0.82 (t, J=7.24 Hz, 1H) 0.66 (t, J=7.45 Hz, 2H)

### Example-66: Synthesis of (S)-4-ethyl-3-(4-methyl-6-((R)-1-(4-(naphthalen-2-yloxy)phenyl)ethylamino)-1,3,5-triazin-2-yl)oxazolidin-2-one (Compound 1.93) and (S)-4-ethyl-3-(4-methyl-6-((S)-1-(4-(naphthalen-2-yloxy)phenyl)ethylamino)-1,3,5-triazin-2-yl)oxazolidin-2-one (Compound 1.94)

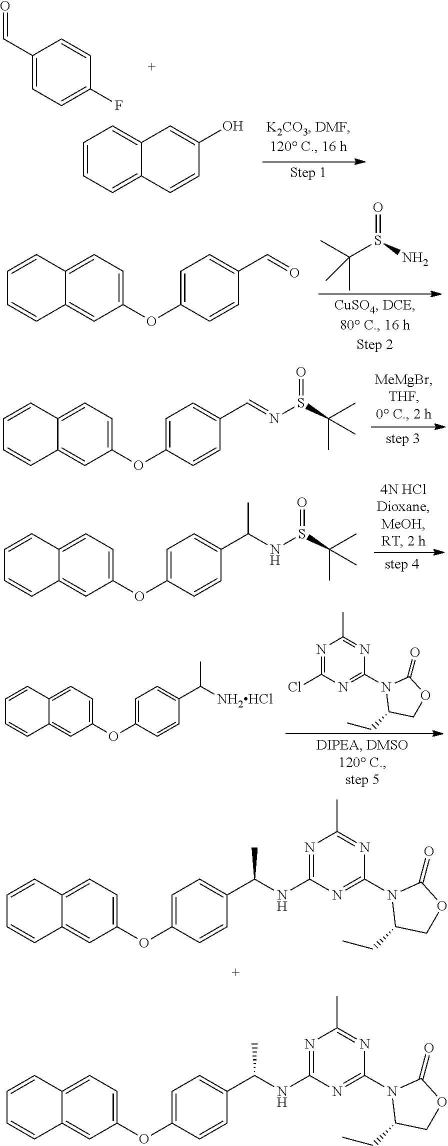

### Step-1: Synthesis of 4-(naphthalen-2-yloxy)benzaldehyde

To a stirred solution of 4-fluorobenzaldehyde (2.0 g, 16.0 mmol, 1.0 eq.), naphthalen-2-ol (2.3 g, 16.0 mmol, 1.0 eq.) in DMF (10 mL) was added K₂CO₃ (4.4 g, 32.0 mmol, 2.0 eq.) at RT. The reaction mixture was heated at 120° C. for 16 h. Following this, reaction was allowed to cool to RT and added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by normal phase silica-gel column chromatography to get the title compound (2.5 g, 63%). LCMS: 249.0 [M+1]⁺; ¹H NMR (400 MHz, CHLOROFORM-d) δ ppm 9.94 (s, 1H) 7.82-7.94 (m, 4H) 7.78 (d, J=7.89 Hz, 1H) 7.43-7.55 (m, 3H) 7.25-7.29 (m, 2H) 7.12 (d, J=8.77 Hz, 2H).

### Step-2: Synthesis of (R,E)-2-methyl-N-(4-(naphthalen-2-yloxy)benzylidene)propane-2-sulfinamide

To a stirred solution of 4-(naphthalen-2-yloxy)benzaldehyde (1.5 g, 6.0 mmol, 1.0 eq.) and Copper(II) sulfate (2.4 g, 15.0 mmol, 2.5 eq.) in dichloroethane (10 mL) was added (R)-2-methylpropane-2-sulfinamide (1.5 g, 12.0 mmol, 2.0 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound (2.0 g, 95%). LCMS: 352.1 [M+1]⁺; ¹H NMR (400 MHz, CHLOROFORM-d) δ ppm 8.55 (s, 1H) 7.82-7.91 (m, 4H) 7.76 (d, J=7.89 Hz, 1H) 7.42-7.54 (m, 3H) 7.23-7.30 (m, 2H) 7.10 (d, J=8.77 Hz, 2H) 1.27 (s, 9H).

### Step-3: Synthesis of (R)-2-methyl-N-(1-(4-(naphthalen-2-yloxy)phenyl)ethyl)propane-2-sulfinamide

To a stirred solution of (R,E)-2-methyl-N-(4-(naphthalen-2-yloxy)benzylidene)propane-2-sulfinamide (1.0 g, 2.8 mmol, 1.0 eq.) in DCM (20 mL) was added drop wise 3 molar methylmagnesium bromide (5.0 mL, 11.3 mmol, 4.0 eq.) at 0° C. The resulting mixture was stirred for 2 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (0.6 g 58%). LCMS: 368.2 [M+1]⁺;

### Step-4: Synthesis of 1-(4-(naphthalen-2-yloxy)phenyl)ethanamine hydrochloride

To a stirred solution of (R)-2-methyl-N-(1-(4-(naphthalen-2-yloxy)phenyl)ethyl)propane-2-sulfinamide (0.6 g, 1.6 mmol, 1.0 eq.) in methanol (10 mL) was added 4N HCl in dioxane (2 mL) at RT. The resulting mixture was stirred for 2 h. Following this, the reaction mixture was evaporated under reduced pressure to get solid. This solid washed with ether and evaporated to give title compound (0.6 g 80%). LCMS: 264.1 [M+1]⁺; ¹H NMR (400 MHz, DMSO-d₆) δ ppm 8.42 (br. s., 2H) 7.92-8.02 (m, 2H) 7.82 (d, J=8.33 Hz, 1H) 7.41-7.58 (m, 6H) 7.28 (dd, J=8.77, 2.19 Hz, 1H) 7.14 (d, J=8.77 Hz, 2H) 4.38-4.47 (m, 1H) 1.52 (d, J=6.58 Hz, 3H).

### Step-5: Synthesis of (S)-4-ethyl-3-(4-methyl-6-((R)-1-(4-(naphthalen-2-yloxy)phenyl)ethylamino)-1,3,5-triazin-2-yl)oxazolidin-2-one and (S)-4-ethyl-3-(4-methyl-6-((S)-1-(4-(naphthalen-2-yloxy)phenyl)ethylamino)-1,3,5-triazin-2-yl)oxazolidin-2-one

In a microwave vial charged with 1-(4-(naphthalen-2-yloxy)phenyl)ethanamine hydrochloride (0.15 g, 0.5 mmol, 1.0 eq.) and (S)-3-(4-chloro-6-methyl-1,3,5-triazin-2-yl)-4-ethyloxazolidin-2-one (0.13 g, 0.5 mmol, 1.0 eq.) in DMSO (5 mL) was added N, N-Diisopropylethylamine (0.2 mL, 1.1 mmol, 2.0 eq.) at RT. The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue. The obtained solid was purified reverse phase column chromatography to get the tile compounds. Compound 1.93 (0.02 g, 8%), UPLC-MS (Method 4): Rt 2.85, m/z 470.5 [M+1]⁺; ¹H NMR (400 MHz, DMSO-d₆) δ ppm 8.53 (d, J=7.89 Hz, 1H) 7.91 (d, J=7.89 Hz, 1H) 7.95 (d, J=8.77 Hz, 1H) 7.81 (d, J=8.33 Hz, 1H) 7.34-7.52 (m, 5H) 7.23-7.30 (m, 1H) 7.00-7.08 (m, 2H) 5.20-5.32 (m, 1H) 5.11 (d, J=7.45 Hz, 1H) 4.48-4.59 (m, 1H) 4.37-4.48 (m, 2H) 4.11-4.19 (m, 1H) 2.17-2.29 (m, 3H) 1.74-1.91 (m, 2H) 1.42-1.49 (m, 3H) 0.80-0.89 (m, 3H). Compound 1.94 (0.015 g, 6%), UPLC-MS (Method 4): Rt 2.83, m/z 470.5 [M+1]⁺; ¹H NMR (400 MHz, DMSO-d₆) δ ppm 8.57 (d, J=8.33 Hz, 1H) 7.90 (d, J=7.89 Hz, 1H) 7.95 (d, J=8.77 Hz, 1H) 7.74-7.84 (m, 1H) 7.36-7.51 (m, 4H) 7.33 (d, J=2.19 Hz, 1H) 7.19-7.29 (m, 1H) 6.99-7.07 (m, 2H) 5.20-5.31 (m, 1H) 5.12 (d, J=7.45 Hz, 1H) 4.49-4.61 (m, 1H) 4.34-4.44 (m, 1H) 4.11-4.18 (m, 1H) 2.17-2.31 (m, 3H) 1.36-1.63 (m, 5H) 0.83 (t, J=7.45 Hz, 1H) 0.74 (t, J=7.45 Hz, 2H).

### Example-67: Synthesis of (S)-3-(4-((S)-1-(4-benzylphenyl)ethylamino)-6-methyl-1,3,5-triazin-2-yl)-4-ethyloxazolidin-2-one (Compound 1.95)

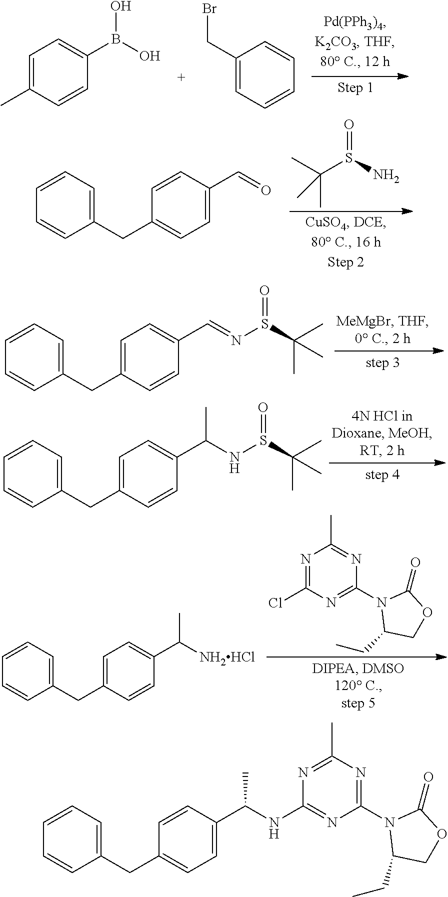

### Step-1: Synthesis of 4-benzylbenzaldehyde

To a stirred solution of 4-formylphenylboronic acid (1.5 g, 10.0 mmol, 1.0 eq.), (bromomethyl)benzene (2.22 g, 13.0 mmol, 1.3 eq.) in THF (15 mL) was added K₂CO₃ (4.5 g, 32.0 mmol, 3.2 eq.) at RT. The reaction mixture purged with nitrogen for 10 min then added Pd(PPh₃)₄ (0.34 g, 0.3 mmol, 0.03 eq.). The reaction mixture was heated at 80° C. for 12 h. Following this, reaction was allowed to cool to RT filtered through celite pad and added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by normal phase silica-gel column chromatography to get the title compound (2.0 g, 98%). LCMS: 196.9 [M+1]⁺; ¹H NMR (400 MHz, CHLOROFORM-d) δ ppm 9.98 (s, 1H) 7.81 (d, J=7.89 Hz, 2H) 7.30-7.38 (m, 3H) 7.15-7.28 (m, 4H) 4.06 (s, 2H).

### Step-2: Synthesis of (R,E)-N-(4-benzylbenzylidene)-2-methylpropane-2-sulfinamide

To a stirred solution of 4-benzylbenzaldehyde (2.0 g, 10.2 mmol, 1.0 eq.) and Copper(II) sulfate (4.0 g, 25.5 mmol, 2.5 eq.) in dichloroethane (20 mL) was added (R)-2-methylpropane-2-sulfinamide (2.2 g, 18.3 mmol, 1.8 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the title compound (1.0 g, 33%). LCMS: 300.1 [M+1]⁺; ¹H NMR (400 MHz, CHLOROFORM-d) δ ppm 8.55 (s, 1H) 7.77 (d, J=8.33 Hz, 2H) 7.12-7.35 (m, 8H) 4.04 (s, 2H) 1.25 (s, 9H).

### Step-3: Synthesis of (R)—N-(1-(4-benzylphenyl)ethyl)-2-methylpropane-2-sulfinamide

To a stirred solution of (R,E)-N-(4-benzylbenzylidene)-2-methylpropane-2-sulfinamide (1.0 g, 3.3 mmol, 1.0 eq.) in DCM (20 mL) was added drop wise 3 molar methylmagnesium bromide (4.5 mL, 13.3 mmol, 4.5 eq.) at 0° C. The resulting mixture was stirred for 2 h at same temperature. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the title compound (1.0 g 96%). LCMS: 316.1 [M+1]⁺; ¹H NMR (400 MHz, CHLOROFORM-d) δ ppm 7.14-7.32 (m, 9H) 4.55 (d, J=5.70 Hz, 1H) 3.97 (s, 2H) 1.52 (d, J=6.58 Hz, 3H) 1.20 (s, 9H).

### Step-4: Synthesis of 1-(4-benzylphenyl)ethanamine hydrochloride

To a stirred solution of (R)—N-(1-(4-benzylphenyl)ethyl)-2-methylpropane-2-sulfinamide (1.0 g, 3.1 mmol, 1.0 eq.) in methanol (10 mL) was added 4N HCl in dioxane (5 mL) at RT. The resulting mixture was stirred for 2 h. Following this, the reaction mixture was evaporated under reduced pressure to get solid. This solid washed with ether and evaporated to give title compound (0.5 g 65%). LCMS: 212.0 [M+1]⁺.

### Step-5: Synthesis of (S)-3-(4-((S)-1-(4-benzylphenyl)ethylamino)-6-methyl-1,3,5-triazin-2-yl)-4-ethyloxazolidin-2-one

In a microwave vial charged with 1-(4-benzylphenyl)ethanamine hydrochloride (0.2 g, 0.94 mmol, 1.0 eq.) and (S)-3-(4-chloro-6-methyl-1,3,5-triazin-2-yl)-4-ethyloxazolidin-2-one (0.23 g, 0.94 mmol, 1.0 eq.) in DMSO (5 mL) was added N, N-Diisopropylethylamine (0.35 mL, 1.8 mmol, 2.0 eq.) at RT. The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue. The obtained solid was purified reverse phase column chromatography to get the tile compounds. Compound 1.95 (0.05 g, 13%), UPLC-MS (Method 7): Rt 3.98, m/z 418.2 [M+1]⁺; ¹H NMR (400 MHz, DMSO-d₆) δ ppm 8.51 (d, J=8.33 Hz, 1H) 7.03-7.33 (m, 9H) 5.12-5.24 (m, 1H) 5.01-5.12 (m, 1H) 4.47 (br. s., 1H) 4.32-4.44 (m, 1H) 4.05-4.17 (m, 1H) 3.83-3.92 (m, 2H) 2.23 (s, 3H) 1.32-1.49 (m, 5H) 0.82 (t, J=7.24 Hz, 1H) 0.64 (t, J=7.45 Hz, 2H).

### Example-68: Synthesis of (S)-3-(4-((S)-1-(5-(4-chlorophenoxy)-6-methylpyridin-2-yl)ethylamino)-6-methyl-1,3,5-triazin-2-yl)-4-ethyloxazolidin-2-one (Compound 1.96) and (S)-3-(4-((R)-1-(5-(4-chlorophenoxy)-6-methylpyridin-2-yl)ethylamino)-6-methyl-1,3,5-triazin-2-yl)-4-ethyloxazolidin-2-one (Compound 1.97)

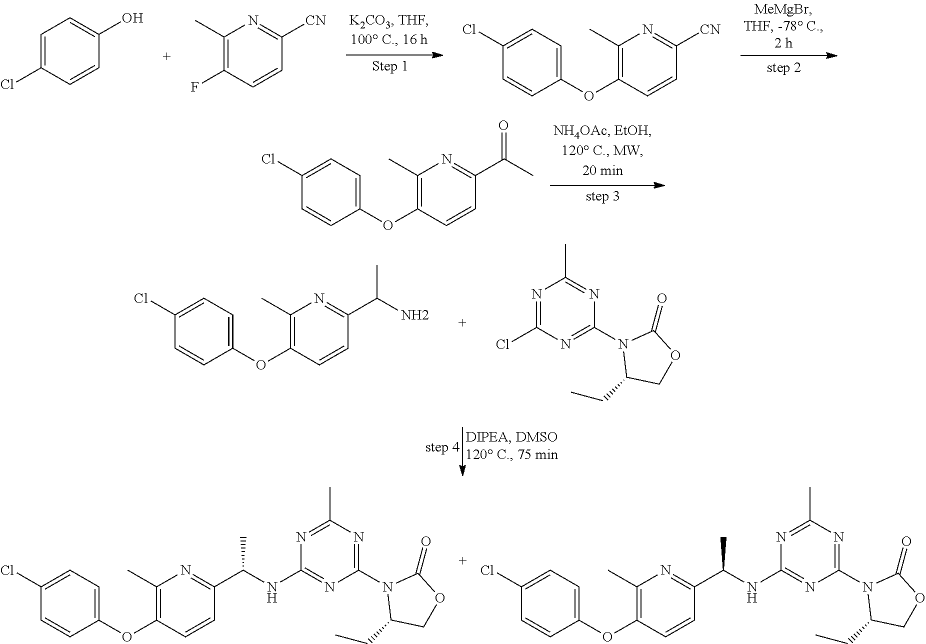

### Step-1: Synthesis of 5-(4-chlorophenoxy)-6-methylpicolinonitrile

To a stirred solution 5-fluoro-6-methylpicolinonitrile (1 g, 7.3 mmol, 1.0 eq.) and 4-chlorophenol (1.13 g, 8.83 mmol, 1.2 eq.) in DMF (10 mL) was added K₂CO₃ (3.0 g, 22.0 mmol, 3 eq.) The resulting mixture heated at 100° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound (1.5 g, 84%). LCMS: 244.9 [M+1]⁺; ¹H NMR (400 MHz, DMSO-d₆) δ ppm 9.65 (s, 1H) 7.85 (m, J=8.77 Hz, 2H) 7.51-7.55 (m, 1H) 7.30 (m, J=8.77 Hz, 1H) 6.75-6.77 (m, 1H) 2.45 (s, 3H).

### Step-2: Synthesis of 1-(5-(4-chlorophenoxy)-6-methylpyridin-2-yl)ethanone

To a stirred solution of 5-(4-chlorophenoxy)-6-methylpicolinonitrile (1.5 g, 6.1 mmol, 1.0 eq.) in THF (20 mL) was added drop wise 3 molar methylmagnesium bromide (8.1 mL, 24.5 mmol, 4.0 eq.) at −78° C. The resulting mixture was stirred for 2 h at same temperature. The reaction was then quenched by careful addition of saturated NH₄Cl (30 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound (0.2 g, 12%). LCMS: 262.0 [M+1]⁺; ¹H NMR (400 MHz, DMSO-d₆) δ ppm 7.82 (d, J=8.77 Hz, 1H) 7.47-7.56 (m, 2H) 7.32 (d, J=8.77 Hz, 1H) 7.12-7.17 (m, 2H) 2.61 (s, 3H) 2.53 (s, 3H).

### Step-3: Synthesis of 1-(5-(4-chlorophenoxy)-6-methylpyridin-2-yl)ethanamine

In a microwave vial charged with 1-(5-(4-chlorophenoxy)-6-methylpyridin-2-yl)ethanone (0.2 g, 0.76 mmol, 1.0 eq.), Ammonium acetate (0.58 g, 7.6 mmol, 10.0 eq.) and sodium cyanoborohydride (0.05 g, 0.76 mmol, 1.0 eq.), in EtOH (10 mL). The resulting mixture was heated at 120° C. for 20 min. Following this, the reaction mixture was allowed to cool to RT, basified with 6N NaOH until pH˜10 and extracted with EtOAc (3×10 mL). The combined organic layers were washed with brine (15 mL), dried over Na₂SO₄ and concentrated under reduced pressure to get the title compound as semi solid which was carried forward without any further purification (0.15 g, 75%). LCMS: 263.0 [M+1]⁺.

### Step-4: Synthesis of (S)-3-(4-((R)-1-(5-(4-chlorophenoxy)-6-methylpyridin-2-yl)ethylamino)-6-methyl-1,3,5-triazin-2-yl)-4-ethyloxazolidin-2-one and (S)-3-(4-((S)-1-(5-(4-chlorophenoxy)-6-methylpyridin-2-yl)ethylamino)-6-methyl-1,3,5-triazin-2-yl)-4-ethyloxazolidin-2-one

In a microwave vial charged with 1-(5-(4-chlorophenoxy)-6-methylpyridin-2-yl)ethanamine (0.15 g, 0.5 mmol, 1.0 eq.), (S)-3-(4-chloro-6-methyl-1,3,5-triazin-2-yl)-4-ethyloxazolidin-2-one (0.14 g, 0.57 mmol, 1.0 eq.) in DMSO (5 mL) was added N, N-Diisopropylethylamine (0.2 g, 1.1 mmol, 2.0 eq.) at RT. The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds. Compound 1.96 (0.009 g, 4%), UPLC-MS (Method 7): Rt 3.54, m/z 469.2 [M+1]⁺; ¹H NMR (400 MHz, DMSO-d₆) δ ppm 8.50 (d, J=7.45 Hz, 1H) 7.41 (d, J=8.77 Hz, 2H) 7.23-7.35 (m, 1H) 7.19 (d, J=8.33 Hz, 1H) 6.86-6.99 (m, 2H) 5.07 (d, J=7.02 Hz, 2H) 4.33-4.59 (m, 2H) 4.10 (d, J=2.63 Hz, 1H) 2.34-2.38 (m, 2H) 2.24-2.28 (m, 2H) 1.47 (d, J=7.02 Hz, 2H) 1.33-1.42 (m, 1H) 0.84 (t, J=7.45 Hz, 1H) 0.63 (t, J=7.45 Hz, 2H), Compound 1.97 (0.013 g, 5%). UPLC-MS (Method 6): Rt 5.15, m/z 469.2 [M+1]⁺; ¹H NMR (400 MHz, DMSO-d₆) δ ppm 8.42 (d, J=7.89 Hz, 1H) 8.27 (d, J=8.77 Hz, 1H) 7.46-7.56 (m, 1H) 7.41 (d, J=8.77 Hz, 1H) 7.24-7.37 (m, 2H) 6.89-6.99 (m, 2H) 5.18-5.31 (m, 1H) 5.04-5.18 (m, 1H) 4.24-4.44 (m, 2H) 4.09-4.20 (m, 1H) 2.34-2.40 (m, 2H) 2.23-2.28 (m, 2H) 1.69-1.87 (m, 2H) 1.47 (d, J=7.02 Hz, 2H) 0.84 (t, J=7.24 Hz, 2H) 0.72 (t, J=7.45 Hz, 1H).

### Example-69: Synthesis of (S)-3-(4-((S)-1-(4-((4-acetylpiperazin-1-yl)methyl)phenyl)ethylamino)-6-methyl-1,3,5-triazin-2-yl)-4-ethyloxazolidin-2-one (Compound 1.98) and (S)-3-(4-((R)-1-(4-((4-acetylpiperazin-1-yl)methyl)phenyl)ethylamino)-6-methyl-1,3,5-triazin-2-yl)-4-ethyloxazolidin-2-one (Compound 1.99)

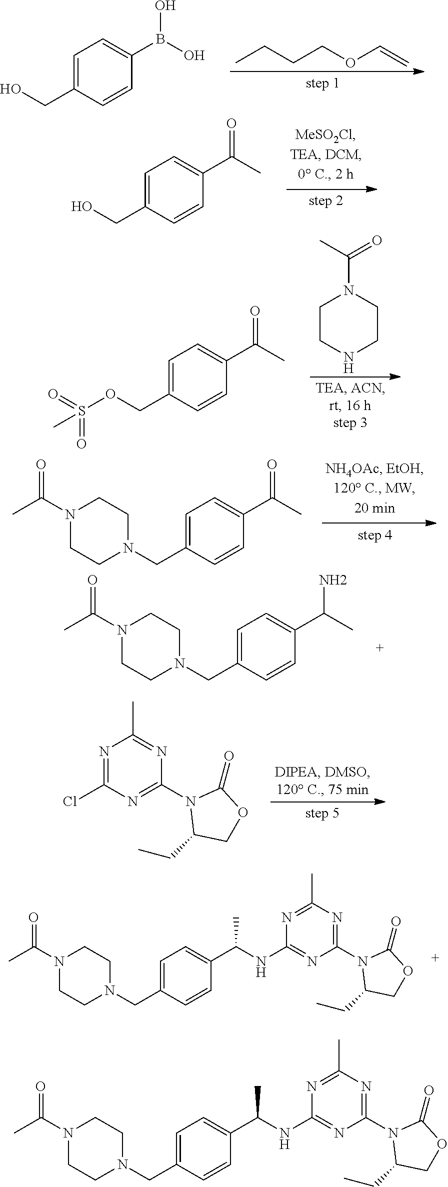

### Step-1: Synthesis of 1-(4-(hydroxymethyl)phenyl)ethanone

To a stirred solution 4-(hydroxymethyl)phenylboronic acid (1.0 g, 6.6 mmol, 1.0 eq.) in acetone (10 mL) was added Pd(OAc)₂ (0.03 g, 0.13 mmol, 0.02 eq.) and DPPP (0.081 g, 0.19 mmol, 0.03 eq.). The reaction mixture purged with nitrogen for 10 min then added 1-(vinyloxy)butane (2.0 mL, 13.2 mmol, 2.0 eq.) The resulting mixture heated at 60° C. for 16 h. Following this, reaction was allowed to cool to RT and added 3N HCl (10 mL) and stirred for 1 h. To the reaction mixture diluted with water (20 mL) and extracted using DCM (3×30 mL). The combined organic layers were washed with brine (50 mL), dried over anhydrous Na₂SO₄, filtered and concentrated under vacuum to get the solid residue. The crude was purified by normal phase silica-gel column provided title compound (0.4 g, 40%). LCMS: 150.9 [M+1]⁺; ¹H NMR (400 MHz, CHLOROFORM-d) δ ppm 7.95 (m, J=8.33 Hz, 2H) 7.46 (m, J=7.89 Hz, 2H) 4.78 (s, 2H) 2.60 (s, 3H).

### Step-2: Synthesis of 4-acetylbenzyl methanesulfonate

To a stirred solution 1-(4-(hydroxymethyl)phenyl)ethanone (0.4 g, 2.6 mmol, 1.0 eq.) in DCM (5 mL) was added methanesulfonyl chloride (0.25 mL, 3.1 mmol, 1.2 eq.) and triethyl amine (0.8 mL, 5.3 mmol, 2 eq.) at 0° C. The resulting mixture stirred for 2 h. After completion of starting material, concentrated under vacuum to get the title compound (0.5 g, 84%), LCMS: 228.9 [M+1]⁺.

### Step-3: Synthesis of 1-(4-(4-acetylbenzyl)piperazin-1-yl)ethanone

To a stirred solution 4-acetylbenzyl methanesulfonate (0.5 g, 3.0 mmol, 1.0 eq.) and 1-(piperazin-1-yl)ethanone (0.6 g, 4.5 mmol, 1.5 eq.) in ACN (10 mL) was added TEA (1.3 mL, 9.0 mmol, 3 eq.) at RT. The resulting mixture stirred for 16 h. Following this, reaction was diluted with water and extracted with ethyl acetate (3×30 mL). The combined organic layers were washed with brine (50 mL), dried over anhydrous Na₂SO₄, filtered and concentrated to get title compound (0.5 g, 64%). LCMS: 261.0 [M+1]⁺.

### Step-4: Synthesis of 1-(4-(4-(1-aminoethyl)benzyl)piperazin-1-yl)ethanone

In a microwave vial charged with 1-(4-(4-acetylbenzyl)piperazin-1-yl)ethanone (0.2 g, 0.76 mmol, 1.0 eq.), Ammonium acetate (0.59 g, 7.6 mmol, 10.0 eq.) and sodium cyanoborohydride (0.05 g, 0.76 mmol, 1.0 eq.), in EtOH (10 mL). The resulting mixture was heated at 120° C. for 20 min. Following this, the reaction mixture was allowed to cool to RT, basified with 6N NaOH until pH˜10 and extracted with EtOAc (3×10 mL). The combined organic layers were washed with brine (15 mL), dried over Na₂SO₄ and concentrated under reduced pressure to get the title compound as semi solid which was carried forward without any further purification (0.15 g, 75%). LCMS: 264.0 [M+1]⁺.

### Step-5: Synthesis of (S)-3-(4-((S)-1-(4-((4-acetylpiperazin-1-yl)methyl)phenyl)ethylamino)-6-methyl-1,3,5-triazin-2-yl)-4-ethyloxazolidin-2-one and (S)-3-(4-((R)-1-(4-((4-acetylpiperazin-1-yl)methyl)phenyl)ethylamino)-6-methyl-1,3,5-triazin-2-yl)-4-ethyloxazolidin-2-one

In a microwave vial charged with 1-(4-(4-(1-aminoethyl)benzyl)piperazin-1-yl)ethanone (0.2 g, 0.76 mmol, 1.0 eq.), (S)-3-(4-chloro-6-methyl-1,3,5-triazin-2-yl)-4-ethyloxazolidin-2-one (0.18 g, 0.76 mmol, 1.0 eq.) in DMSO (5 mL) was added N, N-Diisopropylethylamine (0.3 mL, 1.5 mmol, 2.0 eq.) at RT. The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compounds. Compound 1.98 (0.026 g, 8%), UPLC-MS (Method 2): Rt 1.80, m/z 468.2 [M+1]⁺; ¹H NMR (400 MHz, DMSO-d₆) δ ppm 8.54 (d, J=7.89 Hz, 1H) 7.16-7.39 (m, 4H) 5.02-5.10 (m, 1H) 4.47 (d, J=7.89 Hz, 2H) 4.34-4.43 (m, 1H) 4.08-4.19 (m, 1H) 3.41-3.46 (m, 2H) 2.27-2.36 (m, 1H) 2.24 (s, 4H) 1.96 (s, 3H) 1.87 (br. s., 4H) 1.69-1.80 (m, 2H) 1.36-1.48 (m, 3H) 0.83 (t, J=7.45 Hz, 1H) 0.68 (t, J=7.45 Hz, 2H), Compound 1.99 (0.033 g, 10%). UPLC-MS (Method 2): Rt 1.87, m/z 468.2 [M+1]⁺; ¹H NMR (400 MHz, DMSO-d₆) δ ppm 8.51 (d, J=7.89 Hz, 1H) 7.33 (d, J=8.33 Hz, 2H) 7.19-7.27 (m, 2H) 5.07 (br. s., 2H) 4.35-4.56 (m, 3H) 4.11-4.18 (m, 1H) 3.43 (s, 2H) 2.33 (br. s., 2H) 2.21-2.28 (m, 4H) 1.96 (s, 3H) 1.86 (br. s., 4H) 1.75 (s, 1H) 1.40-1.46 (m, 3H) 0.79-0.88 (m, 3H).

### Example-71: Synthesis of (S)-4-ethyl-3-(4-methyl-6-((S)-1-(3-phenyl-1H-indol-6-yl)ethylamino)-1,3,5-triazin-2-yl)oxazolidin-2-one (Compound 1.100)

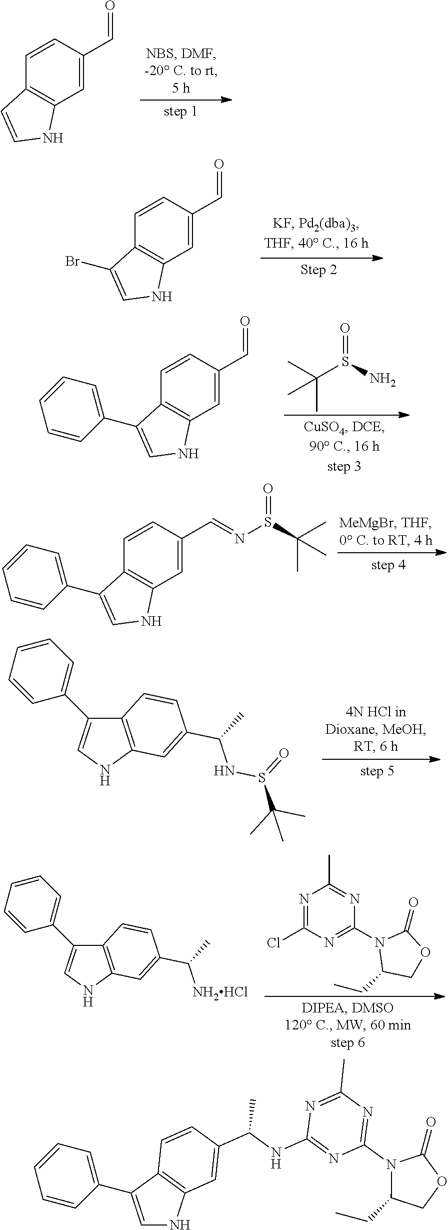

### Step-1: Synthesis of 3-bromo-1H-indole-6-carbaldehyde

To a stirred solution of 1H-indole-6-carbaldehyde (2.0 g, 13.7 mmol, 1.0 eq.) in DMF (35 mL) was added NBS (2.9 g, 16.5 mmol, 1.2 eq.) at −20° C. The reaction mixture allowed to RT stirred for 5 h. Following this, to the reaction mixture added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by normal phase column chromatography to get the desired product (2.4 g, 78%). LCMS: 223.9 [M+1]⁺; ¹H NMR (400 MHz, CHLOROFORM-d) δ ppm 10.08 (s, 1H) 8.63 (br. s., 1H) 7.94 (s, 1H) 7.73 (q, J=8.33 Hz, 2H) 7.47 (d, J=2.63 Hz, 1H).

### Step-2: Synthesis of 3-phenyl-1H-indole-6-carbaldehyde

To a stirred solution of 3-bromo-1H-indole-6-carbaldehyde (1.0 g, 4.4 mmol, 1.0 eq.) and phenylboronic acid (0.65 g, 5.35 mmol, 1.2 eq.) in THF (30 mL) was added KF (0.78 g, 13.38 mmol, 3.0 eq.). The reaction mixture was purged with nitrogen for about 15 min and Pd₂(dba)₃ (0.61 g, 15 mol %) and Tri-tert-butylphosphonium Tetrafluoroborate (0.388 g, 30 mol %) was added. Reaction mixture was re-purged with nitrogen and heated at 40° C. for 16 h. Following this, reaction was allowed to cool to RT and filtered through celite pad, the celite pad washed with ethyl acetate and water. The aqueous layer was separated extracted using ethyl acetate (3×10 mL). The combined organic layers were washed with brine (20 mL), dried over anhydrous Na₂SO₄, filtered and concentrated under vacuum to get the solid residue which was purified by normal phase silica gel column chromatography to get the title compound (0.52 g, 53%). LCMS: 221.9 [M+1]⁺; ¹H NMR (400 MHz, DMSO-d₆) δ ppm 12.00 (br. s., 1H) 10.04 (s, 1H) 7.97-8.07 (m, 3H) 7.68-7.74 (m, 2H) 7.63 (dd, J=8.33, 1.32 Hz, 1H) 7.46 (t, J=7.67 Hz, 2H) 7.23-7.32 (m, 1H).

### Step-3: Synthesis of (R,E)-2-methyl-N-((3-phenyl-1H-indol-6-yl)methylene)propane-2-sulfinamide

To a stirred solution of 3-phenyl-1H-indole-6-carbaldehyde (0.6 g, 2.71 mmol, 1.0 eq.) and Copper(II) sulfate (1.3 g, 8.1 mmol, 3.0 eq.) in dichloroethane (10 mL) was added (R)-2-methylpropane-2-sulfinamide (0.65 g, 5.42 mmol, 2.1 eq.) at RT. The resulting mixture was heated at 90° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the desired product (0.6 g, 68%). LCMS: 325.1 [M+1]⁺; ¹H NMR (400 MHz, DMSO-d₆) δ ppm 11.83 (br. s., 1H) 8.62 (s, 1H) 8.03 (s, 1H) 7.92-7.99 (m, 2H) 7.71 (d, J=7.89 Hz, 3H) 7.45 (t, J=7.89 Hz, 2H) 7.24-7.31 (m, 1H) 1.13-1.25 (m, 9H).

### Step-4: Synthesis of (R)-2-methyl-N—((S)-1-(3-phenyl-1H-indol-6-yl)ethyl)propane-2-sulfinamide

To a stirred solution of (R,E)-2-methyl-N-((3-phenyl-1H-indol-6-yl)methylene)propane-2-sulfinamide (0.6 g, 1.85 mmol, 1.0 eq.) in DCM (5 mL) was added drop wise 3 molar methylmagnesium bromide (4.9 mL, 14.8 mmol, 8.0 eq.) at 0° C. The resulting mixture was allowed to RT and stirred for 4 h. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the desired product (0.3 g 49%). LCMS: 341.0 [M+1]⁺

### Step-5: Synthesis of (S)-1-(3-phenyl-1H-indol-6-yl)ethanamine hydrochloride

To a stirred solution of (R)-2-methyl-N—((S)-1-(3-phenyl-1H-indol-6-yl)ethyl)propane-2-sulfinamide (0.3 g, 0.88 mmol, 1.0 eq.) in methanol (10 mL) was added 4N HCl in dioxane (2 mL) at RT. The resulting mixture was stirred for 6 h. Following this, the reaction mixture was evaporated under reduced pressure to get solid. This solid washed with ether and evaporated to give title compound (0.2 g 83%). LCMS: 237.1 [M+1]⁺

### Step-6: Synthesis of (S)-4-ethyl-3-(4-methyl-6-((S)-1-(3-phenyl-1H-indol-6-yl)ethylamino)-1,3,5-triazin-2-yl)oxazolidin-2-one

In a microwave vial charged with (S)-1-(3-phenyl-1H-indol-6-yl)ethanamine hydrochloride (0.2 g, 0.73 mmol, 1.0 eq.) and (S)-3-(4-chloro-6-methyl-1,3,5-triazin-2-yl)-4-ethyloxazolidin-2-one (0.17 g, 0.73 mmol, 1.0 eq.) in DMSO (3 mL) was added N, N-Diisopropylethylamine (0.5 mL, 2.9 mmol, 4.0 eq.) at RT. The resulting mixture was heated at 120° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound (0.016 g, 5%), UPLC-MS (Method 6): Rt 5.70, m/z 443.2 [M+1]⁺; ¹H NMR (400 MHz, DMSO-d₆) δ ppm 11.27 (br. s., 1H) 8.62 (d, J=7.45 Hz, 1H) 7.74-7.84 (m, 1H) 7.55-7.70 (m, 3H) 7.37-7.44 (m, 2H) 7.11-7.24 (m, 2H) 5.36 (br. s., 1H) 5.19-5.30 (m, 1H) 4.46-4.60 (m, 1H) 4.33-4.43 (m, 1H) 4.08-4.17 (m, 1H) 2.25 (s, 2H) 1.35-1.56 (m, 4H) 0.82 (t, J=7.45 Hz, 1H) 0.67 (t, J=7.24 Hz, 2H).

### Example-71: Synthesis of (S)-4-ethyl-3-(4-methyl-6-((S)-1-(1-methyl-3-phenyl-1H-indol-6-yl)ethylamino)-1,3,5-triazin-2-yl)oxazolidin-2-one (Compound 1.101)

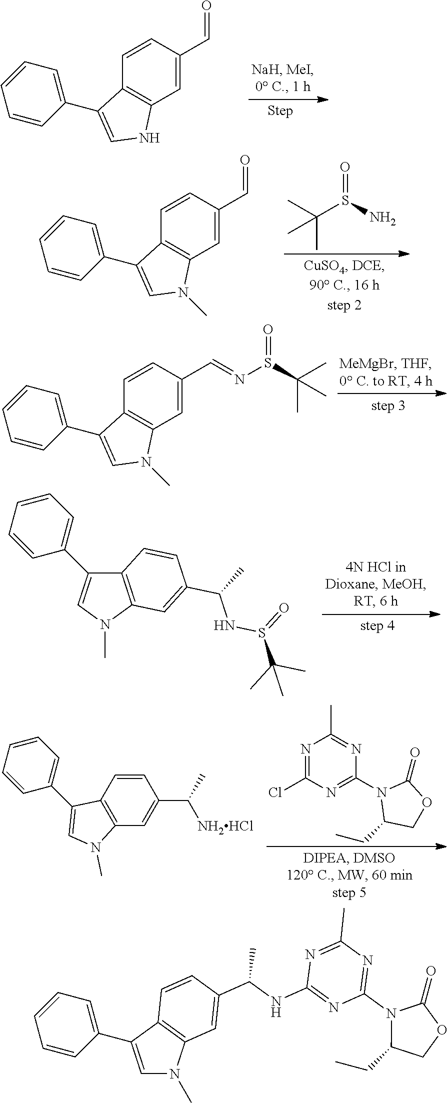

### Step-1: Synthesis of 1-methyl-3-phenyl-1H-indole-6-carbaldehyde

To a stirred solution of 1H-indole-6-carbaldehyde (0.2 g, 0.9 mmol, 1.0 eq.) in THF (10 mL) was added NaH (0.054 g, 1.3 mmol, 1.5 eq.) and MeI (0.19 g, 1.3 mmol, 1.5 eq.) at 0° C. The reaction mixture allowed to RT stirred for 1 h. Following this, to the reaction mixture added water (50 mL) extracted using ethyl acetate (3×10 mL). The combined organic layers were washed with brine (50 mL), dried over anhydrous Na₂SO₄, filtered and concentrated under vacuum to get the solid residue which was purified by was purified by normal phase column chromatography to get the desired product (0.2 g, 94%). ¹H NMR (400 MHz, DMSO-d₆) δ ppm 10.07 (s, 1H) 8.13-8.18 (m, 1H) 7.98-8.06 (m, 2H) 7.64-7.71 (m, 3H) 7.47 (t, J=7.89 Hz, 2H) 7.25-7.32 (m, 1H) 3.97 (s, 3H).

### Step-2: Synthesis of (R,E)-2-methyl-N-((1-methyl-3-phenyl-1H-indol-6-yl)methylene)propane-2-sulfinamide

To a stirred solution of 1-methyl-3-phenyl-1H-indole-6-carbaldehyde (0.4 g, 1.71 mmol, 1.0 eq.) and Copper(II) sulfate (0.67 g, 4.5 mmol, 2.5 eq.) in dichloroethane (15 mL) was added (R)-2-methylpropane-2-sulfinamide (0.43 g, 3.57 mmol, 2.1 eq.) at RT. The resulting mixture was heated at 90° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the desired product (0.3 g, 52%). LCMS: 339.2 [M+1]+

### Step-3: Synthesis of (R)-2-methyl-N—((S)-1-(1-methyl-3-phenyl-1H-indol-6-yl)ethyl)propane-2-sulfinamide

To a stirred solution of (R,E)-2-methyl-N-((1-methyl-3-phenyl-1H-indol-6-yl)methylene)propane-2-sulfinamide (0.3 g, 0.88 mmol, 1.0 eq.) in DCM (10 mL) was added drop wise 3 molar methylmagnesium bromide (3.0 mL, 8.8 mmol, 10.0 eq.) at 0° C. The resulting mixture was allowed to RT and stirred for 4 h. The reaction was then quenched by careful addition of saturated NH₄Cl (20 mL). The aqueous layer was separated and extracted with ethyl acetate (3×30 mL). The combined organic layers were dried over Na₂SO₄, filtered and concentrated to give crude solid residue which was purified by normal phase silica-gel column chromatography to get the desired product (0.25 g 80%). LCMS: 355.2 [M+1]⁺

### Step-4: Synthesis of (S)-1-(1-methyl-3-phenyl-1H-indol-6-yl)ethanamine hydrochloride

To a stirred solution of (R)-2-methyl-N—((S)-1-(1-methyl-3-phenyl-1H-indol-6-yl)ethyl)propane-2-sulfinamide (0.2 g, 0.7 mmol, 1.0 eq.) in methanol (5 mL) was added 4N HCl in dioxane (2 mL) at RT. The resulting mixture was stirred for 6 h. Following this, the reaction mixture was evaporated under reduced pressure to get solid. This solid washed with ether and evaporated to give title compound (0.2 g 99%). LCMS: 251.1 [M+1]⁺

### Step-5: Synthesis of (S)-4-ethyl-3-(4-methyl-6-((S)-1-(1-methyl-3-phenyl-1H-indol-6-yl)ethylamino)-1,3,5-triazin-2-yl)oxazolidin-2-one

In a microwave vial charged with (S)-1-(1-methyl-3-phenyl-1H-indol-6-yl)ethanamine hydrochloride (0.2 g, 0.8 mmol, 1.0 eq.) and (S)-3-(4-chloro-6-methyl-1,3,5-triazin-2-yl)-4-ethyloxazolidin-2-one (0.19 g, 0.8 mmol, 1.0 eq.) in DMSO (3 mL) was added N, N-Diisopropylethylamine (0.5 mL, 3.2 mmol, 4.0 eq.) at RT. The resulting mixture was heated at 130° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by normal phase silica-gel column chromatography followed by reversed phase column chromatography to get the title compound (0.15 g, 41%), UPLC-MS (Method 7): Rt 3.96, m/z 457.3 [M+1]⁺; ¹H NMR (400 MHz, DMSO-d₆) δ ppm 8.58 (d, J=8.33 Hz, 1H) 8.42 (d, J=8.77 Hz, 1H) 7.76-7.85 (m, 1H) 7.59-7.71 (m, 3H) 7.49-7.58 (m, 1H) 7.41 (t, J=7.45 Hz, 2H) 7.17 (d, J=8.33 Hz, 1H) 7.22 (d, J=7.02 Hz, 1H) 5.22-5.32 (m, 1H) 4.53 (t, J=7.89 Hz, 1H) 4.34-4.43 (m, 1H) 4.09-4.18 (m, 1H) 3.77-3.85 (m, 3H) 2.21-2.29 (m, 3H) 1.42-1.61 (m, 4H) 0.82 (t, J=7.24 Hz, 1H) 0.69 (t, J=7.24 Hz, 2H).

### Example-72: Synthesis of 4-((R)-1-(4-((S)-4-ethyl-2-oxooxazolidin-3-yl)-6-methyl-1,3,5-triazin-2-ylamino)ethyl)-N-phenylbenzamide (Compound 1.102) and 4-((S)-1-(4-((S)-4-ethyl-2-oxooxazolidin-3-yl)-6-methyl-1,3,5-triazin-2-ylamino)ethyl)-N-phenylbenzamide (Compound 1.103)

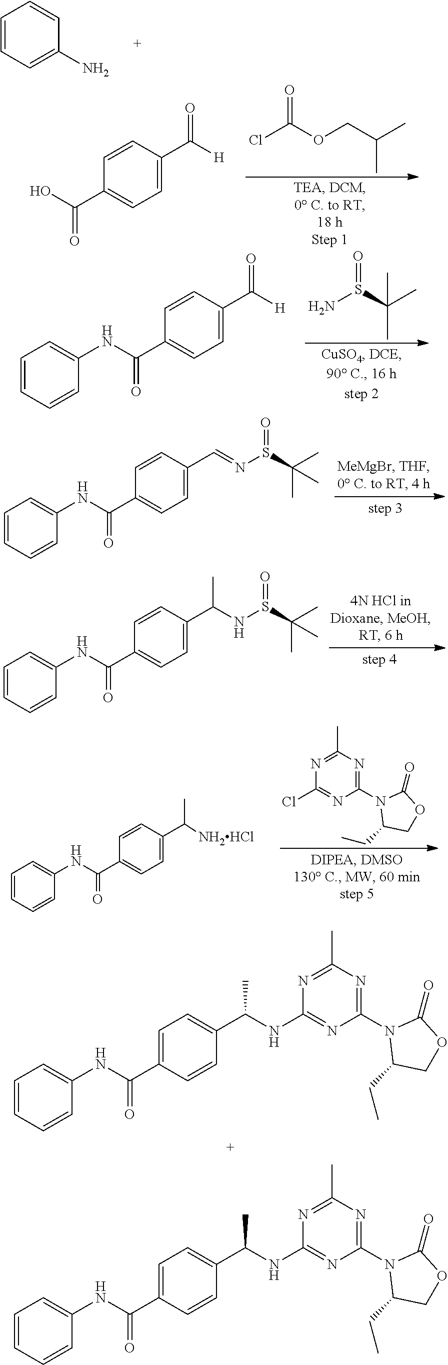

### Step-1: Synthesis of 4-formyl-N-phenylbenzamide

To a cooled solution (0° C.) of 4-formylbenzoic acid (1.1 g, 4.98 mmol, 1.0 eq.) and triethylamine (0.56 g, 5.4 mmol, 1.1 eq.) in CH₂Cl₂ (50 mL) was added isobutyl chloroformate (0.75 g, 5.4 mmol, 1.1 eq.) drop wise. The reaction mixture was stirred for 40 min at 5° C. Phenylamine (0.51 g, 5.4 mmol, 1.1 eq.) was then added, and the solution was stirred for 18 h at room temperature. CH₂Cl₂ (50 mL) was added, and the organics were washed with water (1×75 mL) and brine (1×75 mL), dried over Na₂SO₄, and filtered. The CH₂Cl₂ layer was evaporated under reduced pressure. The crude was purified by normal phase column to get title compound (0.75 g, 68%). LCMS: 226.1 [M+1]⁺

### Step-2: Synthesis of (R,E)-4-(((tert-butylsulfinyl)imino)methyl)-N-phenylbenzamide

To a stirred solution of 4-formyl-N-phenylbenzamide (0.759 g, 3.37 mmol, 1.0 eq.) and Copper(II) sulfate (2.15 g, 13.48 mmol, 4.0 eq.) in dichloroethane (50 mL) was added (R)-2-methylpropane-2-sulfinamide (1.02 g, 8.42 mmol, 2.5 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by flash column chromatography to get the desired product (0.4 g, 36.3%). LCMS: 329.1 [M+1]⁺

### Step-3: Synthesis of 44-(1-(((R)-tert-butylsulfinyl)amino)ethyl)-N-phenylbenzamide

To a stirred solution of (R,E)-4-(((tert-butylsulfinyl)imino)methyl)-N-phenylbenzamide (0.1 g, 0.304 mmol, 1.0 eq.) in DCM (5 mL) was added drop wise 1.5 molar methylmagnesium bromide (2 mL, 3.05 mmol, 10 eq.) at 0° C. The resulting mixture was allowed to come to ambient temperature and stirred for 1 h at same temperature. The reaction was then quenched by careful addition of saturated NH₄Cl (10 mL). The aqueous layer was separated and extracted with DCM acetate (30 mL×3). The combined organic layers were dried over Na₂SO₄, filtered and concentrated to give crude solid residue which was purified by flash column chromatography to get the desired product (0.132 g, Crude) which was used as such for further reaction. LCMS: 345.3 [M+1]⁺

### Step-4: Synthesis of 4-(1-aminoethyl)-N-phenylbenzamide hydrochloride

To a stirred solution of mixture of 4-(1-((S)-1,1-dimethylethylsulfinamido)ethyl)-N-phenylbenzamide and 4-(1-((R)-1,1-dimethylethylsulfinamido)ethyl)-N-phenylbenzamide (0.132 g, 0.383 mmol, 1.0 eq.) in methanol (5 mL) was added 4N HCl in dioxane (5 mL) at RT. The resulting mixture was stirred for 1 h. Following this, the reaction mixture was evaporated under reduced pressure. This solid was washed with ether and dried to give title compound (0.082 g, 77.3%). LCMS: 241.0 [M+1]⁺

### Step-5: Synthesis of 4-((S)-1-((4-((S)-4-ethyl-2-oxooxazolidin-3-yl)-6-methyl-1,3,5-triazin-2-yl)amino)ethyl)-N-phenylbenzamide and 4-((R)-1-((4-((S)-4-ethyl-2-oxooxazolidin-3-yl)-6-methyl-1,3,5-triazin-2-yl)amino)ethyl)-N-phenylbenzamide

In a microwave vial charged with mixture of 4-(1-((S)-1,1-dimethylethylsulfinamido)ethyl)-N-phenylbenzamide and 4-(1-((R)-1,1-dimethylethylsulfinamido)ethyl)-N-phenylbenzamide (0.082 g, 0.31 mmol, 1.0 eq.) and (S)-3-(4-chloro-6-methyl-1,3,5-triazin-2-yl)-4-ethyloxazolidin-2-one (0.114 g, 0.47 mmol, 1.5 eq.) in DMSO (2 mL) was added N, N-Diisopropylethylamine (0.2 mL, 0.94 mmol, 3.0 eq.) at RT. The resulting mixture was heated at 140° C. for 1 h. Following this, the reaction mixture was allowed to cool to RT, diluted with water (5 mL) and solid was filtered off, washed with water (10 mL×2) and dried under vacuum to get the solid residue which was purified by normal phase silica-gel column chromatography followed by reversed phase column chromatography to get the title compounds. Compound 1.102 (0.012 g, 9%), UPLC-MS (Method 6): Rt 3.94, m/z 447.2 [M+1]⁺; ¹H NMR (400 MHz, DMSO-d₆) δ ppm 10.12-10.21 (m, 1H) 8.66 (d, J=7.45 Hz, 1H) 7.89 (d, J=7.89 Hz, 2H) 7.75 (m, J=7.89 Hz, 2H) 7.52 (m, J=8.33 Hz, 2H) 7.34 (t, J=7.89 Hz, 2H) 7.07-7.12 (m, 1H) 5.09-5.16 (m, 1H) 4.35 (d, J=3.07 Hz, 1H) 4.12-4.19 (m, 1H) 2.21-2.28 (m, 3H) 2.09 (s, 2H) 1.72-1.93 (m, 3H) 1.47 (d, J=7.02 Hz, 3H) 0.85 (q, J=7.16 Hz, 3H). Compound 1.103 (0.034 g, 25%), UPLC-MS (Method 4): Rt 2.56, m/z 447.2 [M+1]⁺; ¹H NMR (400 MHz, DMSO-d₆) δ ppm 10.11-10.18 (m, 1H) 8.65 (d, J=7.89 Hz, 1H) 7.88 (d, J=8.33 Hz, 2H) 7.75 (d, J=7.89 Hz, 2H) 7.46-7.55 (m, 2H) 7.34 (t, J=7.67 Hz, 2H) 7.09 (t, J=7.45 Hz, 1H) 5.16 (d, J=7.45 Hz, 1H) 4.49 (t, J=7.89 Hz, 1H) 4.33-4.43 (m, 1H) 4.10-4.18 (m, 1H) 2.19-2.29 (m, 3H) 1.69-1.85 (m, 1H) 1.42-1.51 (m, 3H) 0.83 (t, J=7.45 Hz, 1H) 0.71 (t, J=7.45 Hz, 2H)

### Example-73: Synthesis of (S)-3-(4-((S)-1-(6-chloro-7-isopropoxy-2-oxo-1,2-dihydroquinolin-3-yl)ethylamino)-6-methyl-1,3,5-triazin-2-yl)-4-ethyloxazolidin-2-one (Compound 1.104) and (S)-3-(4-((R)-1-(6-chloro-7-isopropoxy-2-oxo-1,2-dihydroquinolin-3-yl)ethylamino)-6-methyl-1,3,5-triazin-2-yl)-4-ethyloxazolidin-2-one (Compound 1.105)

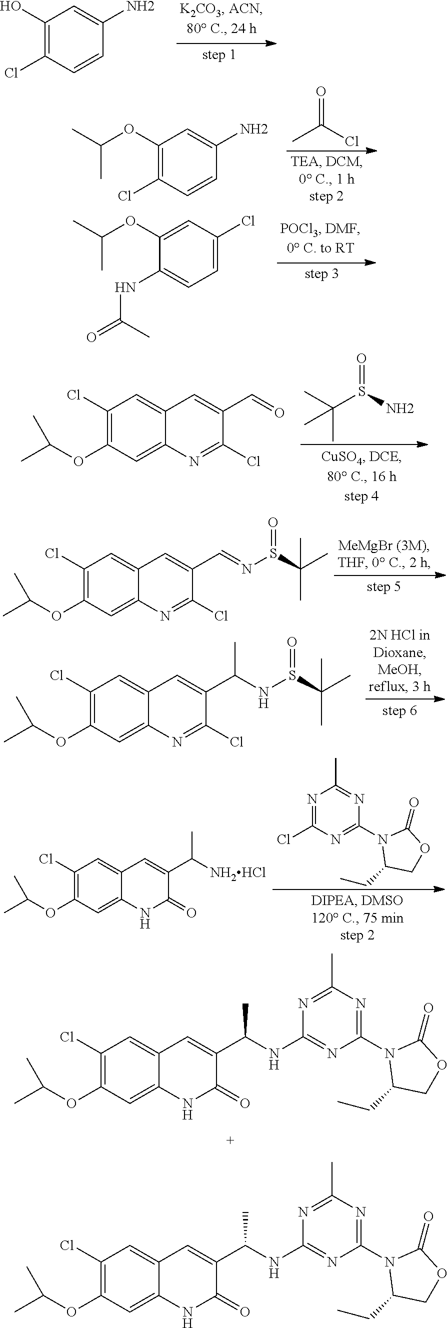

### Step-1: Synthesis of 4-chloro-3-isopropoxyaniline

To a stirred solution 5-amino-2-chlorophenol (5 g, 34.8 mmol, 1.0 eq.) and 2-bromopropane (8.5 g, 69.6 mmol, 2.0 eq.) in ACN (30 mL) was added K₂CO₃ (9.6 g, 69.6 mmol, 2 eq.) The resulting mixture heated at 80° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound (3.0 g, 47%). LCMS: 186.0 [M+1]⁺.

### Step-2: Synthesis of N-(4-chloro-2-isopropoxyphenyl)acetamide

To a stirred solution 4-chloro-3-isopropoxyaniline (4.5 g, 24.72 mmol, 1.0 eq.) in DCM (20 mL) was added Triethyl amine (10.0 mL, 72.24 mmol, 3.0 eq.). The resulting mixture was cooled to 0° C. and added acetyl chloride (2.0 mL, 26.5 mmol, 1.1 eq.) drop wise. The resulting mixture stirred for 1 h at same temperature. Following this, reaction mixture diluted with water (20 mL). The aqueous layer was separated extracted with DCM (3×30 mL). The combined organic layers were washed with brine (50 mL), dried over anhydrous Na₂SO₄, filtered and concentrated under vacuum to get title compound (6.5 g, 56%). LCMS: 228.0 [M+1]⁺. ¹H NMR (400 MHz, DMSO-d₆) δ ppm 10.09 (br. s., 1H) 7.51 (d, J=1.75 Hz, 1H) 7.29 (d, J=8.77 Hz, 1H) 7.13 (dd, J=8.77, 2.19 Hz, 1H) 4.45-4.54 (m, 1H) 1.30 (d, J=6.14 Hz, 6H).

### Step-3: Synthesis of 2,6-dichloro-7-isopropoxyquinoline-3-carbaldehyde

DMF (6.6 mL, 85.0 mmol, 3.0 eq.) was cooled to 0° C. and added POCl₃ (26.6 mL, 286.1 mmol, 10.0 eq.) drop wise over 5 min. To the above solution added N-(4-chloro-2-isopropoxyphenyl)acetamide (6.5 g, 28.6 mmol, 1.0 eq.) dissolved in DMF (10 mL). The resulting mixture heated at 100° C. for 16 h. Following this, reaction mixture diluted with ice cold water (100 mL) and stirred for 1 h. Filtered the solid and under vacuum to get title compound (2.0 g 25%). ¹H NMR (400 MHz, DMSO-d₆) δ ppm 10.33 (s, 1H) 8.85 (s, 1H) 8.45 (s, 1H) 7.64 (s, 1H) 5.01-5.06 (m, 1H) 1.40 (d, J=6.14 Hz, 6H).

### Step-4: Synthesis of (R,E)-N-((2,6-dichloro-7-isopropoxyquinolin-3-yl)methylene)-2-methylpropane-2-sulfinamide

To a stirred solution of 2,6-dichloro-7-isopropoxyquinoline-3-carbaldehyde (0.35 g, 1.76 mmol, 1.0 eq.) and Copper(II) sulfate (0.7 g, 4.4 mmol, 2.5 eq.) in dichloroethane (15 mL) was added (R)-2-methylpropane-2-sulfinamide (0.38 g, 3.1 mmol, 1.8 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the desired product (0.35 g, 53%). LCMS: 387.1 [M+1]⁺. ¹H NMR (400 MHz, DMSO-d₆) δ ppm 9.00 (s, 1H) 8.86 (s, 1H) 8.45 (s, 1H) 7.62 (s, 1H) 4.99-5.05 (m, 1H) 1.40 (d, J=6.14 Hz, 6H) 1.22-1.25 (m, 9H).

### Step-5: Synthesis of (R)—N-(1-(2,6-dichloro-7-isopropoxyquinolin-3-yl)ethyl)-2-methylpropane-2-sulfinamide

To a stirred solution of (R,E)-N-((2,6-dichloro-7-isopropoxyquinolin-3-yl)methylene)-2-methylpropane-2-sulfinamide (0.35 g, 0.9 mmol, 1.0 eq.) in DCM (10 mL) was added drop wise 3 molar methylmagnesium bromide (1.5 mL, 3.6 mmol, 1.5 eq.) at 0° C. The resulting mixture was stirred for 2 h at same temperature. The reaction was then quenched by careful addition of saturated NH₄Cl (10 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound as semi solid (0.2 g, 55%). LCMS: 403.2 [M+1]⁺

### Step-6: Synthesis of 3-(1-aminoethyl)-6-chloro-7-isopropoxyquinolin-2(1H)-one hydrochloride

To a stirred solution of (R)—N-(1-(2,6-dichloro-7-isopropoxyquinolin-3-yl)ethyl)-2-methylpropane-2-sulfinamide (0.2 g, 0.5 mmol, 1.0 eq.) in MeOH (5 mL) was added 4N HCl in dioxane (0.35 mL, 1.5 mmol, 3.0 eq.) at RT. The resulting mixture was heated to reflux for 24 h. Following this, the reaction mixture was evaporated under reduced pressure to get title compound which is used to next step without further purification (0.23 g crude). ¹H NMR (400 MHz, DMSO-d₆) δ ppm 12.08 (s, 1H) 8.28 (br. s., 2H) 7.98 (s, 1H) 7.83 (s, 1H) 7.08 (s, 1H) 4.64 (dt, J=11.95, 6.08 Hz, 1H) 4.39 (br. s., 1H) 1.51 (d, J=6.58 Hz, 3H) 1.34-1.40 (m, 6H).

### Step-7: Synthesis of ((S)-3-(4-((R)-1-(6-chloro-7-isopropoxy-2-oxo-1,2-dihydroquinolin-3-yl)ethylamino)-6-methyl-1,3,5-triazin-2-yl)-4-ethyloxazolidin-2-one and (S)-3-(4-((S)-1-(6-chloro-7-isopropoxy-2-oxo-1,2-dihydroquinolin-3-yl)ethylamino)-6-methyl-1,3,5-triazin-2-yl)-4-ethyloxazolidin-2-one

In a microwave vial charged with 3-(1-aminoethyl)-6-chloro-7-isopropoxyquinolin-2(1H)-one hydrochloride (0.20 g, 0.71 mmol, 1.0 eq.), (S)-3-(4-chloro-6-methoxy-1,3,5-triazin-2-yl)-4-ethyloxazolidin-2-one (0.17 g, 0.71 mmol, 1.0 eq.) and N, N-Diisopropylethylamine (0.3 mL, 1.4 mmol, 2.0 eq.), in DMSO (5 mL). The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid. Compound 1.104 (0.054 g, 15%), UPLC-MS (Method 4): Rt 2.53, m/z 487.2 [M+1]⁺; ¹H NMR (400 MHz, DMSO-d₆) δ ppm 11.70 (s, 1H) 8.46 (d, J=7.02 Hz, 1H) 7.72-7.77 (m, 1H) 7.55 (s, 1H) 6.95-7.01 (m, 1H) 5.28 (d, J=7.89 Hz, 1H) 5.03-5.17 (m, 1H) 4.56-4.63 (m, 1H) 4.34-4.41 (m, 1H) 4.06 (d, J=5.70 Hz, 1H) 2.29 (s, 1H) 2.23 (s, 1H) 1.30-1.42 (m, 6H) 0.84 (t, J=7.24 Hz, 1H) 0.54 (t, J=7.45 Hz, 2H), Compound 1.105 (0.007 g, 2%). UPLC-MS (Method 7): Rt 3.62, m/z 487.2 [M+1]⁺; ¹H NMR (400 MHz, DMSO-d₆) δ ppm 8.35 (d, J=7.89 Hz, 1H) 8.18 (d, J=7.45 Hz, 1H) 7.72-7.78 (m, 1H) 7.64-7.72 (m, 1H) 6.95-7.01 (m, 1H) 5.16 (d, J=7.02 Hz, 1H) 4.54-4.65 (m, 1H) 4.32 (d, J=7.45 Hz, 1H) 2.16-2.31 (m, 2H) 1.84 (br. s., 1H) 1.30-1.41 (m, 6H) 1.04 (d, J=5.70 Hz, 1H) 0.84 (q, J=6.87 Hz, 2H).

### Example-74: Synthesis of (S)-3-(4-((S)-1-(6-(4-chlorophenyl)-2-oxo-1,2-dihydroquinolin-3-yl)ethylamino)-6-methyl-1,3,5-triazin-2-yl)-4-ethyloxazolidin-2-one (Compound 1.106) and (S)-3-(4-((R)-1-(6-(4-chlorophenyl)-2-oxo-1,2-dihydroquinolin-3-yl)ethylamino)-6-methyl-1,3,5-triazin-2-yl)-4-ethyloxazolidin-2-one (Compound 1.107)

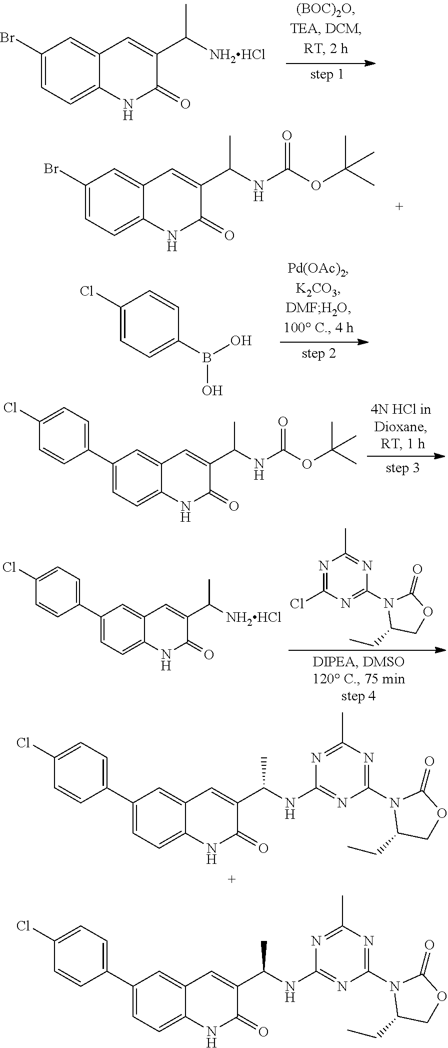

### Step-1: Synthesis of tert-butyl 1-(6-bromo-2-oxo-1,2-dihydroquinolin-3-yl)ethylcarbamate

To a stirred solution 3-(1-aminoethyl)-6-bromoquinolin-2(1H)-one hydrochloride (0.1 g, 0.33 mmol, 1.0 eq.) and di-tert-butyl dicarbonate (0.1 g, 0.5 mmol, 1.5 eq.) in DCM (5 mL) was added TEA (0.1 g, 1.0 mmol, 3 eq.) at RT. The resulting mixture stirred for 2 h. Following this, concentrated under vacuum to get the solid residue. The crude was purified by normal phase silica-gel column provided title compound (0.1 g, 78%). LCMS: 367.0 [M+1]⁺.

### Step-2: Synthesis of tert-butyl 1-(6-(4-chlorophenyl)-2-oxo-1,2-dihydroquinolin-3-yl)ethylcarbamate

To a stirred solution of tert-butyl 1-(6-bromo-2-oxo-1,2-dihydroquinolin-3-yl)ethylcarbamate (0.15 g, 0.4 mmol, 1.0 eq.) and 4-chlorophenylboronic acid (0.095 g, 0.61 mmol, 1.5 eq.) in dmf:water (4:1 mL) was added K₂CO₃ (0.11 g, 0.80 mmol, 2.0 eq.). The reaction mixture was purged with nitrogen for about 15 min and Pd(OAc)₂ (0.004 g, 5 mol %) was added. Reaction mixture was re-purged with nitrogen and heated at 100° C. for 4 h. Following this, reaction was allowed to cool to RT and filtered through celite pad, the celite pad washed with ethyl acetate and water. The aqueous layer was separated extracted using ethyl acetate (3×10 mL). The combined organic layers were washed with brine (20 mL), dried over anhydrous Na₂SO₄, filtered and concentrated under vacuum to get the solid residue which was purified by normal phase silica gel column chromatography to get the title compound (0.1 g, 62%). LCMS: 399.1 [M+1]⁺;

### Step-3: Synthesis of 3-(1-aminoethyl)-6-(4-chlorophenyl)quinolin-2(1H)-one hydrochloride

To a stirred solution of tert-butyl 1-(6-(4-chlorophenyl)-2-oxo-1,2-dihydroquinolin-3-yl)ethylcarbamate (0.1 g, 0.25 mmol, 1.0 eq.) in MeOH (5 mL) was added 4N HCl in dioxane (2 Ml) at RT. The resulting mixture was stirred for 1 h. Following this, the reaction mixture was evaporated under reduced pressure to get title compound which is used to next step without further purification (0.08 g, 93%). LCMS: 299.2 [M+1]⁺.

### Step-4: Synthesis of Synthesis of (S)-3-(4-((S)-1-(6-(4-chlorophenyl)-2-oxo-1,2-dihydroquinolin-3-yl)ethylamino)-6-methyl-1,3,5-triazin-2-yl)-4-ethyloxazolidin-2-one (Compound 1.107) and (S)-3-(4-((R)-1-(6-(4-chlorophenyl)-2-oxo-1,2-dihydroquinolin-3-yl)ethylamino)-6-methyl-1,3,5-triazin-2-yl)-4-ethyloxazolidin-2-one (Compound 1.108)

In a microwave vial charged with 3-(1-aminoethyl)-6-(4-chlorophenyl)quinolin-2(1H)-one hydrochloride (0.08 g, 0.71 mmol, 1.0 eq.), (S)-3-(4-chloro-6-methoxy-1,3,5-triazin-2-yl)-4-ethyloxazolidin-2-one (0.063 g, 0.26 mmol, 1.1 eq.) and N, N-Diisopropylethylamine (0.86 g, 0.71 mmol, 3.0 eq.), in DMSO (3 mL). The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid. Compound 1.106 (0.004 g, 4%), UPLC-MS (Method 7): Rt 3.77, m/z 505.1 [M+1]⁺; ¹H NMR (400 MHz, DMSO-d₆) δ ppm 11.92-11.99 (m, 1H) 8.67 (br. s., 1H) 7.98 (s, 1H) 7.78-7.85 (m, 1H) 7.69-7.77 (m, 2H) 7.46-7.54 (m, 1H) 7.36-7.42 (m, 1H) 5.06-5.18 (m, 1H) 4.07 (d, J=6.14 Hz, 1H) 2.32 (s, 2H) 2.25 (s, 1H) 1.23-1.48 (m, 3H) 0.85 (t, J=7.24 Hz, 1H) 0.55 (t, J=7.45 Hz, 2H), Compound 1.107 (0.015 g, 13%). UPLC-MS (Method 7): Rt 3.62, m/z 505.1 [M+1]⁺; ¹H NMR (400 MHz, DMSO-d₆) δ ppm 11.93 (br. s., 1H) 8.45 (d, J=7.45 Hz, 1H) 7.96 (s, 1H) 7.70-7.88 (m, 4H) 7.50 (d, J=8.33 Hz, 2H) 7.34-7.42 (m, 1H) 5.28-5.37 (m, 1H) 5.16-5.28 (m, 1H) 4.25-4.44 (m, 2H) 4.18 (dd, J=8.33, 2.63 Hz, 1H) 4.09 (d, J=6.14 Hz, 1H) 2.21-2.31 (m, 3H) 1.65-1.90 (m, 3H) 1.42 (d, J=6.58 Hz, 3H) 0.77-0.91 (m, 3H).

### Example-75: Synthesis of (S)-4-ethyl-3-(4-methyl-6-((S)-1-(6-methyl-2-oxo-1,2-dihydroquinolin-3-yl)ethylamino)-1,3,5-triazin-2-yl)oxazolidin-2-one (Compound 1.108) and (S)-4-ethyl-3-(4-methyl-6-((R)-1-(6-methyl-2-oxo-1,2-dihydroquinolin-3-yl)ethylamino)-1,3,5-triazin-2-yl)oxazolidin-2-one (Compound 1.109)

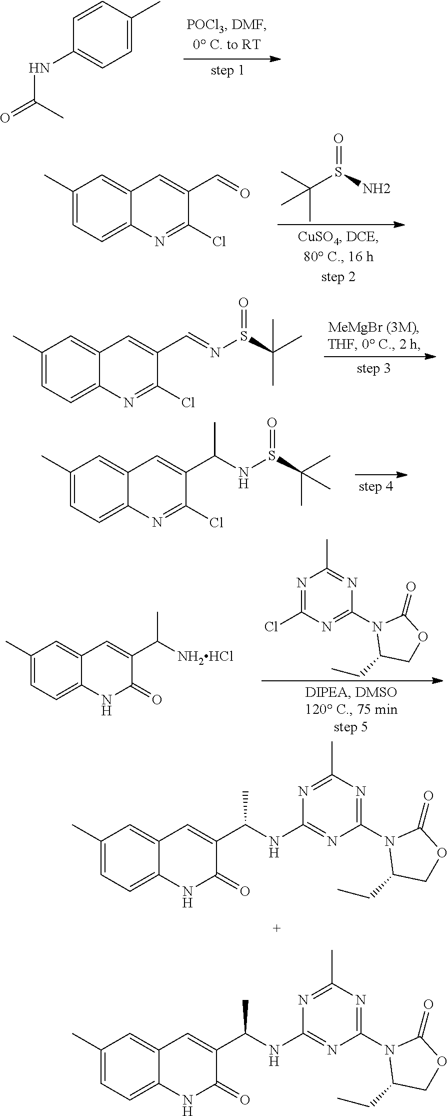

### Step-1: Synthesis of 2-chloro-6-methylquinoline-3-carbaldehyde

DMF (3.3 mL, 40.2 mmol, 4.0 eq.) was cooled to 0° C. and added POCl₃ (9.4 mL, 100.4 mmol, 10.0 eq.) drop wise over 5 min. To the above solution added N-p-tolylacetamide (1.5 g, 10.06 mmol, 1.0 eq.) dissolved in DMF (2 mL). The resulting mixture heated at 80° C. for 16 h. Following this, reaction mixture diluted with ice cold water (50 mL) and stirred for 1 h. Filtered the solid and under vacuum to get title compound (0.8 g 39%). ¹H NMR (400 MHz, DMSO-d₆) δ=10.37 (s, 1H), 8.87 (s, 1H), 8.04 (s, 1H), 7.94 (d, J=8.8 Hz, 1H), 7.83 (dd, J=1.8, 8.3 Hz, 1H), 2.53 (s, 3H)

### Step-2: Synthesis of (R,E)-N-((2-chloro-6-methylquinolin-3-yl)methylene)-2-methylpropane-2-sulfinamide

To a stirred solution of 2-chloro-6-methylquinoline-3-carbaldehyde (0.8 g, 3.9 mmol, 1.0 eq.) and Copper(II) sulfate (1.55 g, 9.7 mmol, 2.5 eq.) in dichloroethane (10 mL) was added (R)-2-methylpropane-2-sulfinamide (0.84 g, 7.02 mmol, 1.8 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the desired product (0.6 g, 50%). LCMS: 309.2 [M+1]⁺. ¹H NMR (400 MHz, DMSO-d₆) δ=9.00 (s, 1H), 8.89 (s, 1H), 8.04 (s, 1H), 7.93 (d, J=8.8 Hz, 1H), 7.79 (dd, J=1.8, 8.8 Hz, 1H), 2.52 (s, 3H), 1.25 (s, 9H).

### Step-3: Synthesis of (R)—N-(1-(2-chloro-6-methylquinolin-3-yl)ethyl)-2-methylpropane-2-sulfinamide

To a stirred solution of R,E)-N-((2-chloro-6-methylquinolin-3-yl)methylene)-2-methylpropane-2-sulfinamide (0.6 g, 1.94 mmol, 1.0 eq.) in DCM (10 mL) was added drop wise 3 molar methylmagnesium bromide (1.0 mL, 2.9 mmol, 1.5 eq.) at 0° C. The resulting mixture was stirred for 2 h at same temperature. The reaction was then quenched by careful addition of saturated NH₄Cl (10 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound as semi solid (0.3 g, 48%). LCMS: 325.2 [M+1]⁺

### Step-4: Synthesis of 3-(1-aminoethyl)-6-methylquinolin-2(1H)-one hydrochloride

To a stirred solution of (R)—N-(1-(2-chloro-6-methylquinolin-3-yl)ethyl)-2-methylpropane-2-sulfinamide (0.3 g, 0.92 mmol, 1.0 eq.) in MeOH (10 mL) was added 4N HCl in dioxane (0.35 mL, 1.3 mmol, 1.5 eq.) at RT. The resulting mixture was heated to reflux for 24 h. Following this, the reaction mixture was evaporated under reduced pressure to get title compound which is used to next step without further purification (0.15 g, 80%). LCMS: 202.9 [M+1]⁺

### Step-5: Synthesis of (S)-4-ethyl-3-(4-methyl-6-((S)-1-(6-methyl-2-oxo-1,2-dihydroquinolin-3-yl)ethylamino)-1,3,5-triazin-2-yl)oxazolidin-2-one and (S)-4-ethyl-3-(4-methyl-6-((R)-1-(6-methyl-2-oxo-1,2-dihydroquinolin-3-yl)ethylamino)-1,3,5-triazin-2-yl)oxazolidin-2-one

In a microwave vial charged with 3-(1-aminoethyl)-6-methylquinolin-2(1H)-one hydrochloride (0.15 g, 0.63 mmol, 1.0 eq.), (S)-3-(4-chloro-6-methoxy-1,3,5-triazin-2-yl)-4-ethyloxazolidin-2-one (0.167 g, 0.69 mmol, 1.1 eq.) and N, N-Diisopropylethylamine (0.2 mL, 1.26 mmol, 2.0 eq.), in DMSO (5 mL). The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid. Compound 1.108 (0.037 g, 14%), UPLC-MS (Method 6): Rt 3.74, m/z 409.1 [M+1]⁺; ¹H NMR (400 MHz, DMSO-d₆) δ=8.43 (d, J=7.0 Hz, 1H), 7.57 (s, 1H), 7.43-7.34 (m, 1H), 7.31-7.16 (m, 2H), 5.37-5.22 (m, 1H), 5.11 (t, J=7.0 Hz, 1H), 4.52-4.31 (m, 2H), 4.10-4.03 (m, 1H), 2.34-2.26 (m, 4H), 1.85-1.75 (m, 1H), 1.40 (d, J=7.0 Hz, 2H), 1.37-1.21 (m, 1H), 0.84 (t, J=7.2 Hz, 1H), 0.53 (t, J=7.5 Hz, 2H), Compound 1.109 (0.034 g, 13%). UPLC-MS (Method 6): Rt 4.02, m/z 409.1 [M+1]⁺; ¹H NMR (400 MHz, DMSO-d₆) δ=11.77 (br. s., 1H), 8.39 (d, J=7.9 Hz, 1H), 8.24 (d, J=8.3 Hz, 1H), 7.74-7.68 (m, 1H), 7.39 (s, 1H), 7.32-7.16 (m, 2H), 5.36-5.27 (m, 1H), 5.21 (d, J=7.0 Hz, 1H), 4.58 (br. s., 1H), 4.43-4.27 (m, 2H), 4.17 (dd, J=2.6, 8.3 Hz, 1H), 4.09 (d, J=5.7 Hz, 1H), 2.35-2.21 (m, 6H), 1.88-1.66 (m, 3H), 1.40 (d, J=6.6 Hz, 3H), 0.89-0.79 (m, 3H).

### Example-76: Synthesis of (S)-4,4-dimethyl-3-(4-methyl-6-(1-(4-phenoxyphenyl)ethylamino)-1,3,5-triazin-2-yl)oxazolidin-2-one (Compound 1.110)

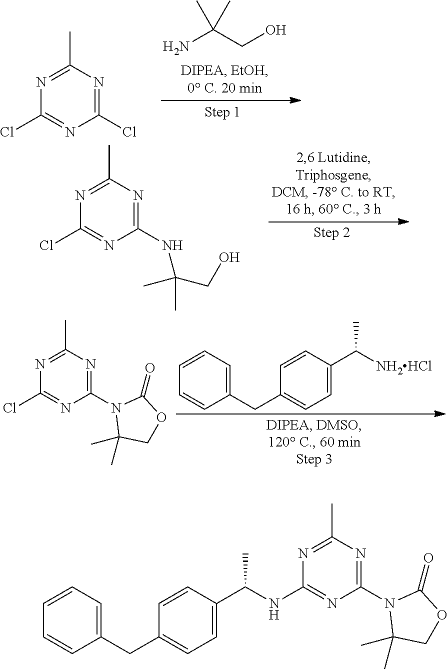

### Step-1: Synthesis of 2-(4-chloro-6-methyl-1,3,5-triazin-2-ylamino)-2-methylpropan-1-ol

To a stirred solution of 2,4-dichloro-6-methyl-1,3,5-triazine (0.9 g, 5.5 mmol, 1.0 eq.), 2-amino-2-methylpropan-1-ol (0.53 g, 6.64 mmol, 1.1 eq.) in EtOH (10 mL) was added DIPEA (1.4 g, 11.8 mmol, 2.0 eq.) at 0° C. The resulting mixture was stirred for 20 min. Following this, concentrated under vacuum to get the solid residue which was purified by was purified flash column to get the desired product as brown color semi solid (0.68 g 57%). LCMS: 216.9 [M+1]⁺

### Step-2: Synthesis of 3-(4-chloro-6-methyl-1,3,5-triazin-2-yl)-4,4-dimethyloxazolidin-2-one

To a stirred solution of 2-(4-chloro-6-methyl-1,3,5-triazin-2-ylamino)-2-methylpropan-1-ol (0.4 g, 1.86 mmol, 1.0 eq.), 2,6 lutidine (0.2 mL, 8.3 mmol, 4.5 eq.) in DCM (10 mL) was added Triphosgene (0.38 g, 1.3 mmol, 0.7 eq.) at −78° C. The resulting mixture allowed to RT and stirred for 16 h and 3 h at 60° C. Following this, concentrated under vacuum to get the solid residue which was purified by was purified flash column to get the desired product as brown color semi solid (0.21 g 47%). LCMS: 243.0 [M+1]⁺

### Step-3: Synthesis of (S)-4,4-dimethyl-3-(4-methyl-6-(1-(4-phenoxyphenyl)ethylamino)-1,3,5-triazin-2-yl)oxazolidin-2-one

In a microwave vial charged with (S)-1-(4-phenoxyphenyl)ethanamine hydrochloride (0.10 g, 0.40 mmol, 1.0 eq.), 3-(4-chloro-6-methyl-1,3,5-triazin-2-yl)-4,4-dimethyloxazolidin-2-one (0.087 g, 0.36 mmol, 0.9 eq.) and N, N-Diisopropylethylamine (0.14 mL, 0.8 mmol, 2.0 eq.), in DMSO (2 mL). The resulting mixture was heated at 150° C. for 60 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified flash column chromatography followed by reversed phase column chromatography to get the desired product as white solid (0.03 g 17%) UPLC-MS (Method 4): Rt 2.63, m/z 420.1 [M+1]⁺; ¹H NMR (400 MHz, DMSO-d₆) δ=8.58 (d, J=7.9 Hz, 1H), 7.42-7.34 (m, 4H), 7.15-7.07 (m, 1H), 7.01-6.91 (m, 4H), 5.04 (t, J=7.2 Hz, 1H), 4.08-4.02 (m, 2H), 2.28-2.24 (m, 3H), 1.58-1.52 (m, 4H), 1.47-1.41 (m, 3H), 1.22 (s, 3H).

### Example-77: Synthesis of (S)-4-ethyl-3-(4-methyl-6-(2-(4-phenoxyphenyl)propan-2-ylamino)-1,3,5-triazin-2-yl)oxazolidin-2-one (Compound 1.111)

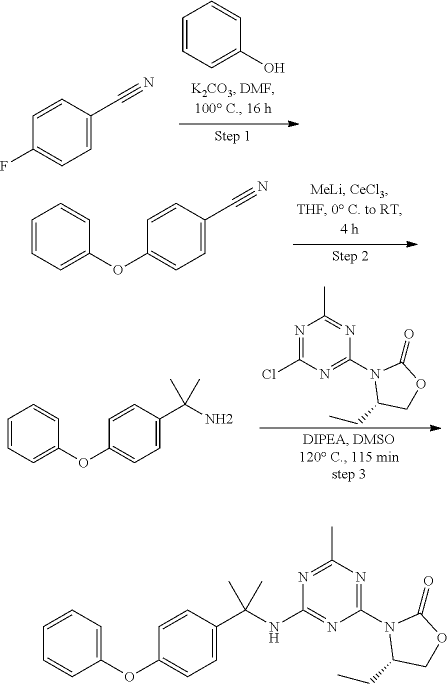

### Step-1: Synthesis of 4-phenoxybenzonitrile

To a stirred solution 4-fluorobenzonitrile (1 g, 16.5 mmol, 1.0 eq.) and phenol (1.7 g, 18.18 mmol, 1.1 eq.) in DMF (20 mL) was added K₂CO₃ (6.8 g, 49.5 mmol, 3 eq.) The resulting mixture heated at 100° C. for 16 h. Following this, reaction was allowed to cool to RT and filtered through celite pad, the celite pad washed with ethyl acetate and water. The aqueous layer was separated extracted using ethyl acetate (3×30 mL). The combined organic layers were washed with brine (50 mL), dried over anhydrous Na₂SO₄, filtered and concentrated under vacuum to get the solid residue. The crude was purified by normal phase silica-gel column provided title compound (1.2 g, 37%). LCMS: 196.1 [M+1]⁺.

### Step-2: Synthesis of 2-(4-phenoxyphenyl)propan-2-amine

THF (3 mL) was added to a Schlenk flask containing CeCl₃ (2.29 g, 9.31 mmol, 6.0 eq.). After 15 minutes of stirring the grey suspension was cooled to −78° C. and MeLi (6.0 mL, 9.31 mmol, 6.0 eq.) was added drop wise over the course of 10 minutes. After 30 minutes a solution of 4-phenoxybenzonitrile (0.3 g, 1.5 mmol, 1.0 eq.) in 2 mL THF was added drop wise to the reaction mixture. After 15 minutes of stirring the reaction mixture was allowed to warm to room temperature and was stirred for an hour. The solution was again cooled to −78° C. and aqueous NH₄OH (6 mL) was added. The mixture was allowed to warm to room temperature overnight. The reaction mixture was decanted and the residue was extracted with THF. Organic fractions were combined and all volatiles were evaporated. The resulting yellow oil was dissolved in DCM, extracted with brine (20 mL), dried over Mg₂SO₄ and all volatiles were evaporated affording a product which was used without further purification (0.11 g, 31%). LCMS: 211.0 [M−NH₃]⁺.

### Step-3: Synthesis of (S)-4-ethyl-3-(4-methyl-6-(2-(4-phenoxyphenyl)propan-2-ylamino)-1,3,5-triazin-2-yl)oxazolidin-2-one

In a microwave vial charged with 2-(4-phenoxyphenyl)propan-2-amine (0.07 g, 0.30 mmol, 1.0 eq.) and (S)-3-(4-chloro-6-methyl-1,3,5-triazin-2-yl)-4-ethyloxazolidin-2-one (0.089 g, 0.36 mmol, 1.1 eq.) in DMSO (2 mL) was added N, N-Diisopropylethylamine (0.16 mL, 0.92 mmol, 3.0 eq.) at RT. The resulting mixture was heated at 120° C. for 115 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue. The obtained solid was purified reverse phase column chromatography to get the tile compounds 1.111 (0.011 g, 8%), UPLC-MS (Method 2): Rt 2.87, m/z 434.2 [M+1]⁺; ¹H NMR (400 MHz, DMSO-d₆) δ=8.37 (s, 1H), 7.40-7.29 (m, 3H), 7.10 (t, J=7.5 Hz, 1H), 6.92 (dd, J=4.6, 8.1 Hz, 3H), 4.26 (s, 1H), 4.06 (d, J=7.9 Hz, 1H), 2.25 (s, 2H), 1.74 (s, 2H), 1.59 (s, 2H), 0.62 (t, J=7.5 Hz, 2H).

### Example-78: Synthesis of (S)-3-(4-((S)-1-(6-chloro-2-oxo-7-(pyridin-2-ylmethoxy)-1,2-dihydroquinolin-3-yl)ethylamino)-6-methyl-1,3,5-triazin-2-yl)-4-ethyloxazolidin-2-one (Compound 1.112) and (S)-3-(4-((R)-1-(6-chloro-2-oxo-7-(pyridin-2-ylmethoxy)-1,2-dihydroquinolin-3-yl)ethylamino)-6-methyl-1,3,5-triazin-2-yl)-4-ethyloxazolidin-2-one (Compound 1.113)

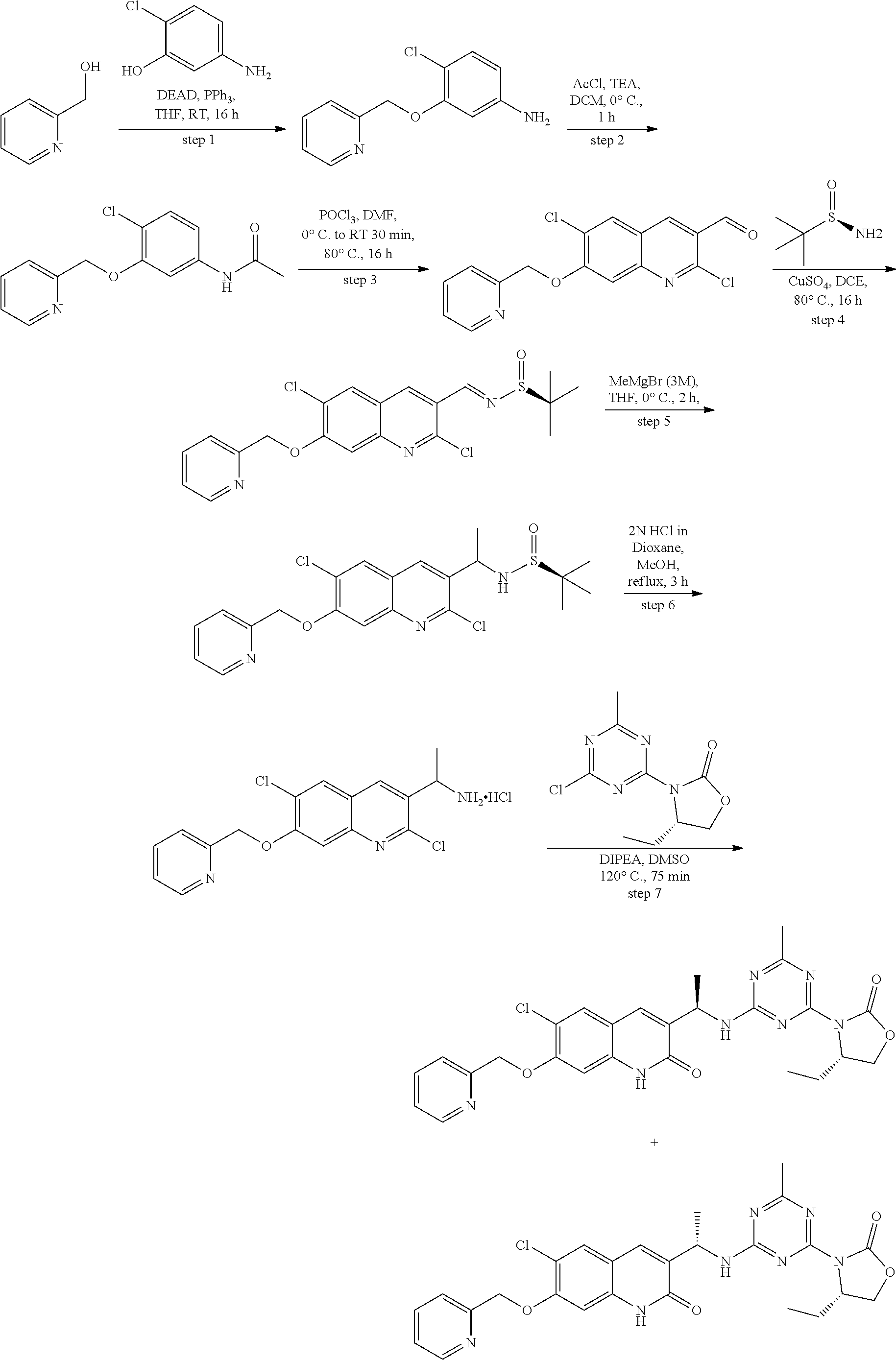

### Step-1: Synthesis of 4-chloro-3-(pyridin-2-ylmethoxy)aniline

To a stirred solution 5-amino-2-chlorophenol (1 g, 6.99 mmol, 1.0 eq.) and pyridin-2-ylmethanol (0.76 g, 6.99 mmol, 1.0 eq.) in THF (15 mL) was added PPh₃ (1.82 g, 10.48 mmol, 1.5 eq.) and DEAD (2.7 g, 10.48 mmol, 1.5 eq.) at RT. The resulting mixture stirred for 16 h. Following this, reaction mature concentrated under vacuum to get the solid residue. The crude was purified by normal phase silica-gel column provided title compound (0.8 g, 49%). LCMS: 234.9 [M+1]⁺.

### Step-2: Synthesis of N-(4-chloro-3-(pyridin-2-ylmethoxy)phenyl)acetamide

To a stirred solution of 4-chloro-3-(pyridin-2-ylmethoxy)aniline (0.8 g, 3.41 mmol, 1.0 eq.) in DCM (10 mL) was added Triethyl amine (0.7 mL, 5.11 mmol, 3.0 eq.). The resulting mixture was cooled to 0° C. and added acetyl chloride (0.8 mL, 10.25 mmol, 3.0 eq.) drop wise. The resulting mixture stirred for 1 h at same temperature. Following this, reaction mixture diluted with water (20 mL). The aqueous layer was separated extracted with DCM (3×30 mL). The combined organic layers were washed with brine (50 mL), dried over anhydrous Na₂SO₄, filtered and concentrated under vacuum to get title compound (0.7 g, 74%). LCMS: 276.9 [M+1]⁺

### Step-3: Synthesis of 2,6-dichloro-7-(pyridin-2-ylmethoxy)quinoline-3-carbaldehyde

DMF (0.6 mL, 7.59 mmol, 3.0 eq.) was cooled to 0° C. and added POCl₃ (2.4 mL, 25.3 mmol, 10.0 eq.) drop wise over 5 min. To the above solution added N-(4-chloro-3-(pyridin-2-ylmethoxy) phenyl)acetamide (0.7 g, 2.53 mmol, 1.0 eq.) dissolved in DMF (2 mL). The resulting mixture heated at 80° C. for 16 h. Following this, reaction mixture diluted with ice cold water (50 mL) and stirred for 1 h. Filtered the solid and under vacuum to get title compound (0.45 g 53%). LCMS: 332.9 [M+1]⁺

### Step-4: Synthesis of (R,E)-N-((2,6-dichloro-7-(pyridin-2-ylmethoxy)quinolin-3-yl)methylene)-2-methylpropane-2-sulfinamide

To a stirred solution of 2,6-dichloro-7-(pyridin-2-ylmethoxy)quinoline-3-carbaldehyde (0.45 g, 1.3 mmol, 1.0 eq.) and Copper(II) sulfate (0.6 g, 3.7 mmol, 2.8 eq.) in dichloroethane (10 mL) was added (R)-2-methylpropane-2-sulfinamide (0.29 g, 2.43 mmol, 1.8 eq.) at RT. The resulting mixture was heated at 80° C. for 16 h. Following this, reaction was allowed to cool to room temperature, filtered through celite pad, the celite pad washed with dichloromethane (20 mL). The combined filtrate dried over anhydrous Na₂SO₄ and concentrated under vacuum to get the solid residue which was purified by normal phase silica-gel column chromatography to get the desired product (0.3 g, 51%). LCMS: 436.0 [M+1]⁺.

### Step-5: Synthesis of (R)—N-(1-(2,6-dichloro-7-(pyridin-2-ylmethoxy)quinolin-3-yl)ethyl)-2-methylpropane-2-sulfinamide

To a stirred solution of (R,E)-N-((2,6-dichloro-7-(pyridin-2-ylmethoxy)quinolin-3-yl)methylene)-2-methylpropane-2-sulfinamide (0.3 g, 0.68 mmol, 1.0 eq.) in DCM (10 mL) was added drop wise 3 molar methylmagnesium bromide (2.3 mL, 6.8 mmol, 10.0 eq.) at 0° C. The resulting mixture was stirred for 2 h at same temperature. The reaction was then quenched by careful addition of saturated NH₄Cl (10 mL). The aqueous layer was separated and extracted with ethyl acetate (3×10 mL). The combined organic layers were dried over Na₂SO₄, filtered and concentrated to give crude solid residue which was purified by normal phase silica-gel column chromatography to get the title compound (0.2 g, 65%). LCMS: 452.0 [M+1]⁺

### Step-6: Synthesis of 3-(1-aminoethyl)-6-chloro-7-(pyridin-2-ylmethoxy)quinolin-2(1H)-one hydrochloride

To a stirred solution of (R)—N-(1-(2,6-dichloro-7-(pyridin-2-ylmethoxy)quinolin-3-yl)ethyl)-2-methylpropane-2-sulfinamide (0.2 g, 0.44 mmol, 1.0 eq.) in MeOH (10 mL) was added 4N HCl in dioxane (1.0 mL.) at RT. The resulting mixture was heated to reflux for 24 h. Following this, the reaction mixture was evaporated under reduced pressure to get title compound which is used to next step without further purification (0.10 g, 62%). LCMS: 330.4 [M+1]⁺

### Step-7: Synthesis of (S)-3-(4-((S)-1-(6-chloro-2-oxo-7-(pyridin-2-ylmethoxy)-1,2-dihydroquinolin-3-yl)ethylamino)-6-methyl-1,3,5-triazin-2-yl)-4-ethyloxazolidin-2-one and (S)-3-(4-((R)-1-(6-chloro-2-oxo-7-(pyridin-2-ylmethoxy)-1,2-dihydroquinolin-3-yl)ethylamino)-6-methyl-1,3,5-triazin-2-yl)-4-ethyloxazolidin-2-one

In a microwave vial charged with 3-(1-aminoethyl)-6-chloro-7-(pyridin-2-ylmethoxy)quinolin-2(1H)-one hydrochloride (0.10 g, 0.3 mmol, 1.0 eq.), (S)-3-(4-chloro-6-methoxy-1,3,5-triazin-2-yl)-4-ethyloxazolidin-2-one (0.075 g, 0.3 mmol, 1.0 eq.) and N, N-Diisopropylethylamine (0.1 mL, 0.6 mmol, 2.0 eq.), in DMSO (5 mL). The resulting mixture was heated at 120° C. for 75 min. Following this, the reaction mixture was allowed to cool to RT, diluted with water (10 mL) and extracted using ethyl acetate (3×10 mL). The combined organic layers were washed with brine (10 mL), dried over anhydrous Na₂SO₄, filtered and concentrated under vacuum to get the solid residue which was purified by was purified normal phase silica-gel column chromatography followed by reversed phase column chromatography to get the title compound as white solid. Compound 1.112 (0.015 g, 9%), UPLC-MS (Method 2): Rt 2.68, m/z 536.5 [M+1]⁺; ¹H NMR (400 MHz, DMSO-d₆) δ=8.59 (br. s., 1H), 8.38 (d, J=6.6 Hz, 1H), 8.18 (d, J=7.0 Hz, 1H), 7.91-7.75 (m, 1H), 7.59-7.46 (m, 1H), 7.36 (br. s., 1H), 7.26 (br. s., 1H), 7.12-6.99 (m, 1H), 6.66 (br. s., 1H), 5.27 (br. s., 2H), 5.16-4.96 (m, 1H), 4.36-4.29 (m, 1H), 4.14 (br. s., 1H), 4.03 (d, J=6.1 Hz, 1H), 2.26 (s, 1H), 2.21 (s, 1H), 1.73 (d, J=6.1 Hz, 4H), 1.37 (d, J=6.6 Hz, 2H), 1.27 (br. s., 1H), 0.83 (t, J=7.2 Hz, 1H), 0.50 (t, J=7.2 Hz, 2H), Compound 1.113 (0.004 g, 3%). UPLC-MS (Method 2): Rt 2.82, m/z 536.5 [M+1]⁺; ¹H NMR (400 MHz, DMSO-d₆) δ=8.61 (br. s., 1H), 8.36 (d, J=7.0 Hz, 1H), 7.91-7.84 (m, 1H), 7.73-7.67 (m, 1H), 7.56 (d, J=7.9 Hz, 1H), 7.40-7.35 (m, 1H), 7.08-7.03 (m, 1H), 5.29 (br. s., 1H), 5.15 (d, J=7.0 Hz, 1H), 4.31 (d, J=7.5 Hz, 1H), 4.09 (d, J=6.1 Hz, 1H), 2.29-2.20 (m, 1H), 1.64 (s, 2H), 1.38 (d, J=6.6 Hz, 1H), 1.23 (br. s., 1H), 1.04 (d, J=6.1 Hz, 1H), 0.94-0.79 (m, 2H).

### Biological Examples

### Example-B1: Mutant IDH1-R132H Fluorescence Assay

IC₅₀ values of compounds against IDH1 mutant (R132H) was determined by Resazurin based Fluorescence assay. Assays were performed in Buffer (25 mM Tris HCl, 150 mM NaCl, 10 mM MgCl2, 0.03% BSA, 2 mM 1-mercaptoethanol) where total reaction volume was 12.5 μL in low-volume 384-well plates (Cat #4511, Corning). Serially diluted compounds (3-fold) were incubated with cocktail of α-ketoglutarate (4 mM; Cat #75892-25G, Sigma), NADPH (10 M; Cat # N1630-100MG, Sigma) and buffer for 5 min, following IDH1 R132H mutant (10 nM; Cat #71099-2, BPS Bioscience) was added and incubated in dark at room temperature for 1.5 h. After 1.5 h, 6.25 μL detection solution containing Resazurin (30 M; (Cat #62758-13-8, Sigma) and Diaphorase (40 ug/ml; Cat # D2197-300UN, Sigma) in buffer was added and incubated for 15 min. Readings were taken in a Synergy Neo Plate reader (BioTek, Winooski) at single excitation of 540 nm and emission at 590 nm respectively.

The % (percent) activity of test samples was calculated as (Sample−Min)×100/(Max−Min). [Max: DMSO control, complete reaction with enzyme with DMSO and Min: No enzyme with DMSO]. Percent inhibition (100−% activity) was fitted to the “four-parameter logistic model” in XLfit for determination of IC₅₀ values.

The results of the Fluorescence assay for Mutant IDH1-R132H are shown in Tables B1.

### Example-B2: IDH1-WT Fluorescence Assay

IC₅₀ values of compounds against IDH1-WT was determined by Resazurin based Fluorescence assay. Assays were performed in Buffer (50 mM Tris HCl, 10 mM MgCl2, 0.03% BSA, 2 mM ß-mercaptoethanol) where total reaction volume was 25 μL in low-volume 96-well plates. Serially diluted compounds (3-fold) in DMSO (0.5% in final reaction) were incubated with cocktail of isocitrate (200 μM; Cat # I1252-1G, Sigma), NADP⁺ (15 μM; Cat # N0505-500MG, Sigma) and buffer for 5 min, following addition of IDH1-WT enzyme (1 nM; Cat #71075-2, BPS Bioscience) and incubated in dark at room temperature for 30 min. After that, 12.5 μL detection solution containing Resazurin (60 μM; Cat #62758-13-8, Sigma) and Diaphorase (40 μg/ml; Cat # D2197-300UN, Sigma) in buffer was added and incubated for 15 min. Readings were taken using a Synergy Neo Plate reader (BioTek, Winooski) at single excitation of 540 nm and emission at 590 nm respectively.

The % (percent) activity of test samples was calculated as (Sample−Min)×100/(Max−Min). [Max: DMSO control, complete reaction with enzyme with DMSO and Min: No enzyme with DMSO]. Percent inhibition (100−% activity) was fitted to the “four-parameter logistic model” in XLfit for determination of IC₅₀ values.

The results of the Fluorescence assay for IDH1 WT are shown in Tables B1.

### Example-B3: Mutant IDH2-R140Q Fluorescence Assay

IC₅₀ values of compounds against IDH2 mutant (R140Q) were determined by Resazurin based Fluorescence assay. Assay was performed in Buffer (25 mM Tris HCl, 150 mM NaCl, 10 mM MgCl₂, 0.03% BSA, 2 mM 1-mercaptoethanol) where total reaction volume was 12.5 μL in low-volume 384-well plates (Cat #4511, Corning). Serially diluted compounds (3-fold) were pre-incubated with IDH2 R140Q mutant enzyme (5 nM; Cat #71100-2, BPS Bioscience) for 4 hr at RT (25° C.) followed by addition of cocktail of α-ketoglutarate (1 mM; Cat #75892-25G, Sigma), and NADPH (5 μM; Cat # N1630-100MG, Sigma) with buffer in dark at room temperature for 2 h. After 2 h incubation, 6.25 μL detection solution containing Resazurin (30 μM; (Cat #62758-13-8, Sigma) and Diaphorase (40 ug/ml; Cat # D2197-300UN, Sigma) in buffer was added and incubated for 15 min. Readings were taken in a Synergy Neo Plate reader (BioTek, Winooski) at single excitation of 540 nm and emission at 590 nm respectively.

The % activity of test samples was calculated as (Sample−Min)×100/(Max−Min). [Max: DMSO control, complete reaction with enzyme with DMSO and Min: No enzyme with DMSO]. Percent inhibition (100−% activity) was fitted to the “four-parameter logistic model” in XLfit for determination of IC₅₀ values.

The results of the Fluorescence assay for Mutant IDH2-R140Q are shown in Tables B1.

TABLE B1
Fluorescence assay for Mutant IDH2- R140Q

| | IDH1(R132H) | IDH1 WT | IDH2 (R140Q) |
|---|---|---|---|
| Compound | IC50 (μM) | IC50 (μM) | IC50 (μM) |
| 1.1 | 2.77 | >50 | >50 |
| 1.2 | >50 | >50 | ND |
| 1.3 | 0.830 | >30 | >30 |
| 1.4 | 3.30 | >30 | >50 |
| 1.5 | 0.575 | >30 | >50 |
| 1.6 | >30 | >30 | ND |
| 1.7 | 2.87 | >30 | >50 |
| 1.8 | >30 | ND | ND |
| 1.9 | 0.642 | >30 | >50 |
| 1.10 | 2.76 | >30 | >50 |
| 1.11 | 11.4 | ND | >50 |
| 1.12 | 9.01 | ND | >50 |
| 1.13 | 0.77 | >30 | >50 |
| 1.14 | 24.5 | ND | ND |
| 1.15 | >30 | >30 | ND |
| 1.16 | 4.35 | >30 | >50 |
| 1.17 | >30 | ND | ND |
| 1.18 | >30 | ND | ND |
| 1.19 | 1.66 | >50 | >50 |
| 1.20 | >30 | ND | ND |
| 1.21 | 1.40 | >50 | >50 |
| 1.22 | >30 | ND | ND |
| 1.23 | 0.360 | >50 | >50 |
| 1.24 | >30 | ND | ND |
| 1.25 | >30 | ND | ND |
| 1.26 | 3.04 | >50 | >50 |
| 1.27 | 0.396 | >50 | >50 |
| 1.28 | 4.52 | >50 | >50 |
| 1.29 | 0.492 | >50 | >50 |
| 1.30 | >30 | ND | ND |
| 1.31 | 0.486 | >30 | >50 |
| 1.32 | 0.313 | >30 | >50 |
| 1.33 | >30 | ND | ND |
| 1.34 | >30 | >30 | >50 |
| 1.35 | 0.868 | >30 | >50 |
| 1.36 | >30 | ND | ND |
| 1.37 | 19.4 | >50 | >50 |
| 1.38 | 0.144 | >50 | >50 |
| 1.39 | >30 | ND | ND |
| 1.40 | 0.298 | >50 | >50 |
| 1.41 | 0.707 | >50 | >50 |
| 1.42 | 0.084 | >50 | >50 |
| 1.43 | 6.83 | ND | ND |
| 1.44 | 0.209 | >50 | >50 |
| 1.45 | 1.79 | ND | ND |
| 1.46 | >30 | ND | ND |
| 1.47 | 3.18 | ND | ND |
| 1.48 | >30 | ND | ND |
| 1.49 | >30 | ND | ND |
| 1.50 | 0.456 | >50 | >50 |
| 1.51 | >22.9 | ND | ND |
| 1.52 | 0.258 | >50 | >50 |
| 1.53 | >30 | ND | >50 |
| 1.54 | 1.21 | ND | ND |
| 1.55 | 1.25 | ND | ND |
| 1.56 | >30 | ND | ND |
| 1.57 | 2.07 | ND | ND |
| 1.58 | 0.308 | >50 | >50 |
| 1.59 | 0.178 | >50 | >50 |
| 1.60 | >30 | ND | ND |
| 1.61 | 17.0 | ND | ND |
| 1.62 | 3.29 | ND | ND |
| 1.63 | 2.40 | ND | ND |
| 1.64 | 0.227 | >50 | >50 |
| 1.65 | 2.65 | ND | ND |
| 1.66 | 0.328 | >50 | >50 |
| 1.67 | 12.3 | ND | ND |
| 1.68 | 1.58 | ND | ND |
| 1.69 | >30 | ND | ND |
| 1.70 | >30 | ND | ND |
| 1.71 | 2.44 | ND | ND |
| 1.72 | >30 | ND | ND |
| 1.73 | 15.4 | ND | ND |
| 1.74 | >30 | ND | ND |
| 1.75 | 0.832 | >50 | >50 |
| 1.76 | >23.3 | ND | ND |
| 1.77 | 5.95 | ND | ND |
| 1.78 | 0.440 | >50 | >50 |
| 1.79 | 2.72 | ND | ND |
| 1.80 | 10.2 | ND | ND |
| 1.81 | 0.586 | >50 | >50 |
| 1.82 | 0.414 | >50 | >50 |
| 1.83 | 0.340 | >50 | >50 |
| 1.84 | 0.615 | >50 | >50 |
| 1.85 | 0.914 | >50 | >50 |
| 1.86 | 0.277 | >50 | >50 |
| 1.87 | 8.82 | ND | ND |
| 1.88 | >30 | ND | ND |
| 1.89 | 1.42 | ND | ND |
| 1.90 | 1.54 | ND | ND |
| 1.91 | >30 | ND | ND |
| 1.92 | 0.749 | >50 | >50 |
| 1.93 | >30 | ND | ND |
| 1.94 | 0.520 | >50 | >50 |
| 1.95 | 2.64 | ND | ND |
| 1.96 | 3.83 | ND | ND |
| 1.97 | >30 | ND | ND |
| 1.98 | 21.4 | ND | ND |
| 1.99 | >30 | ND | ND |
| 1.100 | 2.24 | ND | ND |
| 1.101 | 13.6 | ND | ND |
| 1.102 | >30 | ND | ND |
| 1.103 | >30 | ND | ND |
| 1.104 | 0.075 | >50 | >50 |
| 1.105 | 0.660 | >50 | >50 |
| 1.106 | >30 | ND | ND |
| 1.107 | >30 | ND | ND |
| 1.108 | 1.27 | ND | ND |
| 1.109 | >30 | ND | ND |
| 1.110 | 2.02 | ND | ND |
| 1.111 | 2.51 | ND | ND |

* = ND Not Determined

### Example-B4: Cellular 2-HG Assay Using Mutant IDHJ (R132C) HT1080 Cells

HT1080 (ATCC® CCL-121™) cells were plated in a 12 well plate (Nunc™ Cell-Culture Treated Multidish; Cat #150628) at a cell density of 5×10⁴ cells/well. Cells were then treated with various concentrations of of test/reference compounds (ranging from 0.003 to 3 μM) or vehicle for 48 h under cell culture conditions (in CO₂ incubator, 5% CO₂, 37° C.). Metabolites were extracted from cell culture media by precipitation method and IC₅₀ values of compounds were determined by Resazurin based Fluorescence assay. Assay buffer contained 100 mM HEPES pH 8.0, 100 μM NAD⁺ (Sigma; Cat # N1630-100MG), 0.1 lg HGDH, 5 μM resazurin (Cat #62758-13-8, Sigma) and 0.01 U/ml diaphorase (Cat # D2197-300UN, Sigma). Total assay volume was 100 μl. 75 μl of assay buffer was added to 25 μl sample volume (supernatant) and incubated at RT in the dark for 30 min in black 96-well plates (Thermo Scientific). Fluorometric detection was done using a Synergy Neo Plate reader (BioTek, Winooski) at single excitation of 540 nm and emission at 590 nm respectively).

The % activity of test samples was calculated as (Sample−Min)×100/(Max−Min). [Max: DMSO control, complete reaction with enzyme with DMSO and Min: No enzyme with DMSO]. Percent inhibition (100−% activity) was fitted to the “four-parameter logistic model” in XLfit for determination of IC₅₀ values.

The results of the Cellular 2-HG assay using mutant IDH1 (R132C) HT1080 cells are shown in Tables B2.

TABLE B2
Cellular 2-HG assay using mutant IDH1 (R132C) HT1080

| | 2-HG IC₅₀ in IDH1 (R132C) |
|---|---|
| Compound No. | HT1080 cells (μM) |
| 1.38 | 0.634 |
| 1.42 | 0.556 |
| 1.44 | 0.168 |
| 1.50 | 0.413 |
| 1.59 | 0.271 |
| 1.64 | 0.036 |
| 1.104 | 0.094 |

It is understood that the foregoing examples and embodiments described above are for illustrative purposes only and that various modifications or changes in light thereof will be suggested to persons skilled in the art and are to be included within the spirit and purview of this application and the scope of the appended claims.

What is claimed is:

1. A compound of Formula (IA): 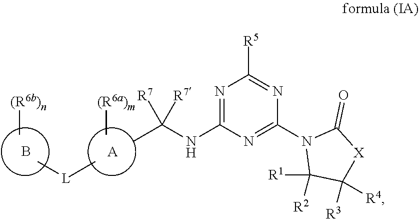
or a salt, polymorph, solvate, enantiomer, stereoisomer or tautomer thereof, wherein
wherein,
X is O, S, NR^{a} or CR^{b}R^{c};
A is C₆-C₁₀ aryl, 5- to 10 membered heteroaryl, C₃-C₈ cycloalkyl or 3- to 10-membered heterocyclyl, wherein each of which is optionally substituted by R^{6a};
B is hydrogen, C₆-C₁₀ aryl, 5- to 10-membered heteroaryl, C₃-C₈ cycloalkyl or 3- to 10-membered heterocyclyl, wherein each of which is optionally substituted by R^{6b};
L is a bond, —O—, —(CH₂)₁₋₃—, —NH—, —NCH₃—, —SO₂—, —C(O)—, —CH₂—O—, —S—, —CR^{b}R^{c}—, —C(O)NH— or —NHC(O)—;
R^{a} is hydrogen or C₁-C₆ alkyl optionally substituted by oxo, —OH or halogen;
R^{b} and R^{c} are independently hydrogen, halogen, —CN, C₁-C₆ alkyl, C₃-C₆ cycloalkyl, or —(C₁-C₃ alkylene)(C₃-C₆ cycloalkyl);
R¹ is hydrogen, halogen or C₁-C₆ alkyl;
R² is hydrogen, halogen, C₁-C₆ alkoxy, C₁-C₆ haloalkoxy, —C(O)OR^{2a}, C₃-C₆ cycloalkyl, 3- to 6-membered heterocyclyl, C₆ aryl, 5- to 6-membered heteroaryl, —(C₁-C₃ alkylene)C₆ aryl or C₁-C₆ alkyl optionally substituted by oxo, halogen, —OR^{2a} or —NR^{2a}R^{2b}, wherein C₃-C₆ cycloalkyl, 3- to 6-membered heterocyclyl, C₆ aryl, 5- to 6-membered heteroaryl, —(C₁-C₃ alkylene)C₆ aryl of R² optionally substituted by C₁-C₆ alkyl;
or R¹ and R² are taken together with the atom to which they are attached to form a C₃-C₆ cycloalkyl or 3- to 6-membered heterocyclyl, each of which is optionally substituted by oxo, —OH, halogen, —NH₂, or C₁-C₆ alkyl optionally substituted by oxo, —OH, halogen or —NH₂;
R^{2a} and R^{2b} are independently hydrogen or C₁-C₆ alkyl;
R³ and R⁴ are independently hydrogen, halogen, or C₁-C₆ alkyl optionally substituted by oxo, —OH or halogen;
or R³ and R⁴ are taken together with the atom to which they are attached to form a C₃-C₆ cycloalkyl or 3- to 6-membered heterocyclyl, each of which is optionally substituted by oxo, —OH, -halogen, —NH₂, or C₁-C₆ alkyl optionally substituted by oxo, —OH, halogen or —NH₂;
R⁵ is hydrogen, C₁-C₆ alkyl, C₂-C₆ alkenyl, C₂-C₆ alkynyl, C₃-C₆ cycloalkyl, 3- to 6-membered heterocyclyl, C₆ aryl, 5- to 6-membered heteroaryl, —CN, halogen, C₁-C₆ alkoxy, C₁-C₆ haloalkoxy, —OR¹⁰, —SR¹⁰, —S(O)₂R¹⁰, —S(O)₂NR¹¹R¹², —NR¹⁰S(O)₂R¹¹, —NR¹¹R¹², —C(O)R¹⁰, —NR¹⁰C(O)R¹¹, —NR¹⁰C(O)NR¹¹R¹², —C(O)OR¹⁰, —C(O)ONR¹¹R¹², —C(O)NR¹¹R¹², wherein each of which is optionally substituted by R⁸;
each R^{6a} and R^{6b} is independently oxo, C₁-C₆ alkyl, C₂-C₆ alkenyl, C₂-C₆ alkynyl, C₃-C₆ cycloalkyl, 3- to 6-membered heterocyclyl, C₆ aryl, —CN, halogen, C₁-C₆ alkoxy, C₁-C₆ haloalkoxy, C₁-C₆ haloalkyl, —OR¹³, —SR¹³, —S(O)₂R¹³, —S(O)₂NR¹⁴R¹⁵, —NR¹³S(O)₂R¹⁴, —NR¹⁴R¹⁵, —C(O)R¹³, —NR¹³C(O)R¹⁴, —NR¹³C(O)NR¹⁴R¹⁵, —C(O)OR¹³, —C(O)ONR¹⁴R¹⁵, —C(O)NR¹⁴R¹⁵, —(C₁-C₃ alkylene)OR¹³, —(C₁-C₃ alkylene)SR¹³, —(C₁-C₃ alkylene)S(O)₂R¹³, —(C₁-C₃ alkylene)S(O)₂NR¹⁴R¹⁵, —(C₁-C₃ alkylene)NR¹³S(O)₂R¹⁴, —(C₁-C₃ alkylene)NR¹⁴R¹⁵, —(C₁-C₃ alkylene)C(O)R¹³, —(C₁-C₃ alkylene)NR¹³C(O)R¹⁴, —(C₁-C₃ alkylene)NR¹³C(O)NR¹⁴R¹⁵, —(C₁-C₃ alkylene)C(O)OR¹³, —(C₁-C₃ alkylene)C(O)ONR¹⁴R¹⁵, —(C₁-C₃ alkylene)(C₃-C₈ cycloalkyl) or —(C₁-C₃ alkylene)(3- to 10-membered heterocyclyl); wherein each of R^{6a} and R^{6b} is independently optionally substituted by oxo, C₂-C₆ alkenyl, C₂-C₆ alkynyl, —CN, halogen, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶, C₁-C₆ alkyl optionally substituted by oxo, OH, halogen or NH₂;
R⁷ and R^{7′} are independently hydrogen, C₃-C₆ cycloalkyl or C₁-C₆ alkyl optionally substituted by halogen or —OH;
or R⁷ and R^{7′} are taken together with the atom to which they are attached to form a C₃-C₆ cycloalkyl;
R⁸ is halogen, oxo, C₂-C₆ alkenyl, C₂-C₆ alkynyl, C₃-C₆ cycloalkyl 3- to 6-membered heterocyclyl, —CN, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶ or C₁-C₆ alkyl optionally substituted by oxo, —OH, halogen or NH₂;
each R¹⁰, R¹¹ and R¹² is independently hydrogen, C₁-C₆ alkyl, C₂-C₆ alkenyl, C₂-C₆ alkynyl, C₃-C₆ cycloalkyl or 3- to 6-membered heterocyclyl, wherein each of R¹⁰, R¹¹ and R¹² is independently optionally substituted by oxo, C₂-C₆ alkenyl, C₂-C₆ alkynyl, —CN, halogen, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶ or C₁-C₆ alkyl optionally substituted by oxo, OH, halogen or NH₂;
or R¹¹ and R¹² are taken together with the atom to which they attached to form a 3-6 membered heterocyclyl optionally substituted by oxo, OH, halogen, NH₂, or C₁-C₆ alkyl optionally substituted by oxo, OH, halogen or NH₂;
each R¹³, R¹⁴ and R¹⁵ is independently hydrogen, C₁-C₆ alkyl, C₂-C₆ alkenyl, C₂-C₆ alkynyl, C₃-C₆ cycloalkyl, 3- to 6-membered heterocyclyl, —(C₁-C₃ alkylene)C₃-C₆ cycloalkyl or —(C₁-C₃ alkylene) 5- to 6-heteroaryl, wherein each of R¹³, R¹⁴ and R¹⁵ is independently optionally substituted by oxo, C₂-C₆ alkenyl, C₂-C₆ alkynyl, —CN, halogen, C₁-C₆ alkoxy, C₁-C₆ haloalkoxy, —OR¹⁶, —SR¹⁶, —S(O)₂R¹⁶, —S(O)₂NR¹⁷R¹⁸, —NR¹⁶S(O)₂R¹⁷, —NR¹⁷R¹⁸, —C(O)R¹⁶, —NR¹⁶C(O)R¹⁷, —C(O)OR¹⁶ or C₁-C₆ alkyl optionally substituted by oxo, OH, halogen or NH₂;
or R¹⁴ and R¹⁵ are taken together with the atom to which they attached to form a 3- to 6-membered heterocyclyl optionally substituted by oxo, OH or halogen, or C₁-C₆ alkyl optionally substituted by oxo, OH, halogen or NH₂;
each R¹⁶, R¹⁷ and R¹⁸ is independently hydrogen, C₂-C₆ alkenyl, C₂-C₆ alkynyl, or C₁-C₆ alkyl optionally substituted by oxo, OH, halogen or NH₂;
or R¹⁷ and R¹⁸ are taken together with the atom to which they attached to form a 3- to 6-membered heterocyclyl optionally substituted by oxo, OH, halogen or NH₂, or C₁-C₆ alkyl optionally substituted by oxo, OH, halogen or NH₂;
m and n is independently 0, 1, 2, 3 or 4.

2. The compound of claim 1, wherein X is O.

3. The compound of claim 1, wherein X is S.

4. The compound of claim 1, wherein X is NR^{a}.

5. The compound of claim 4, wherein R^{a} is selected from hydrogen or methyl.

6. The compound of claim 1, wherein X is CR^{b}R^{c}.

7. The compound of claim 6, wherein R^{b} and R^{c} are independently selected from hydrogen, halogen, —CN, methyl or cylcopropyl.

8. The compound of claim 1, wherein A is selected from C₆-C₁₀ aryl or 5- to 10 membered heteroaryl each of which is optionally substituted by R^{6a}.

9. The compound of claim 1, wherein A is selected from 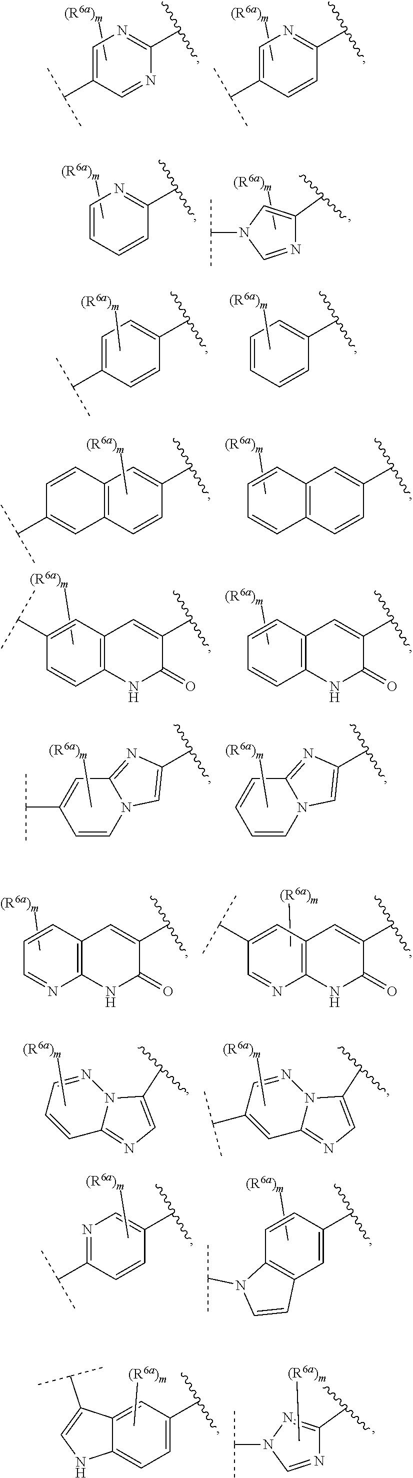 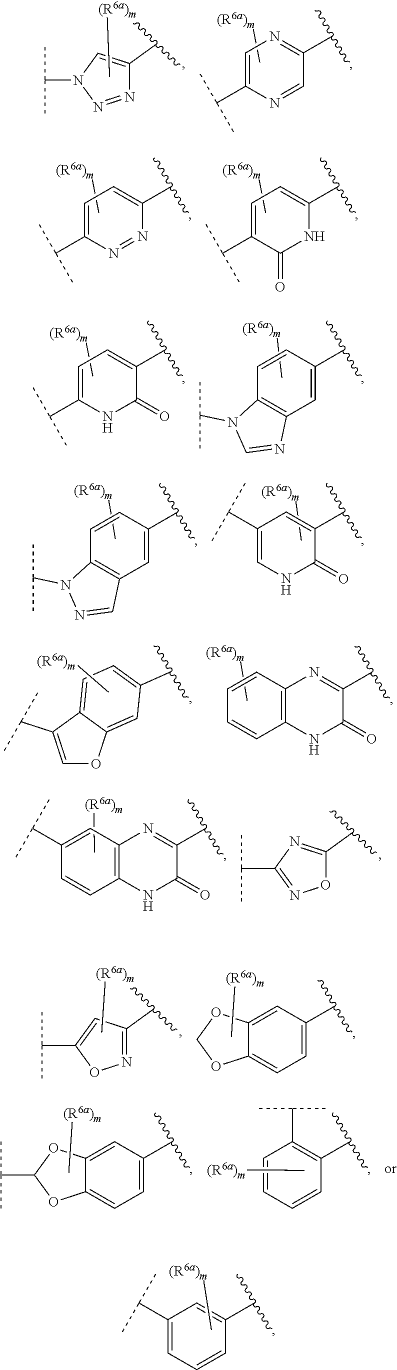
wherein wavy line indicates attachment points to the alkylamine and dotted line indicates attachment points to the L.

10. The compound of claim 9, wherein m is 0, 1, 2, 3 or 4.

11. The compound of claim 1, wherein R^{6a} is selected from oxo, halogen, —CN, C₁-C₆ alkyl, C₁-C₆ alkoxy, C₁-C₆ haloalkoxy, C₁-C₆ haloalkyl, —OR¹³ or C₆-aryl optionally substituted by halogen.

12. The compound of claim 11, wherein R^{6a} is selected from oxo, —CN, —Cl, —F, —Br, methyl, —OCH₃, —CF₃, —CH₂CHF₂, —OCF₃, chlorophenyl or phenyl.

13. The compound of claim 1, wherein A optionally substituted with R^{6a} is selected from 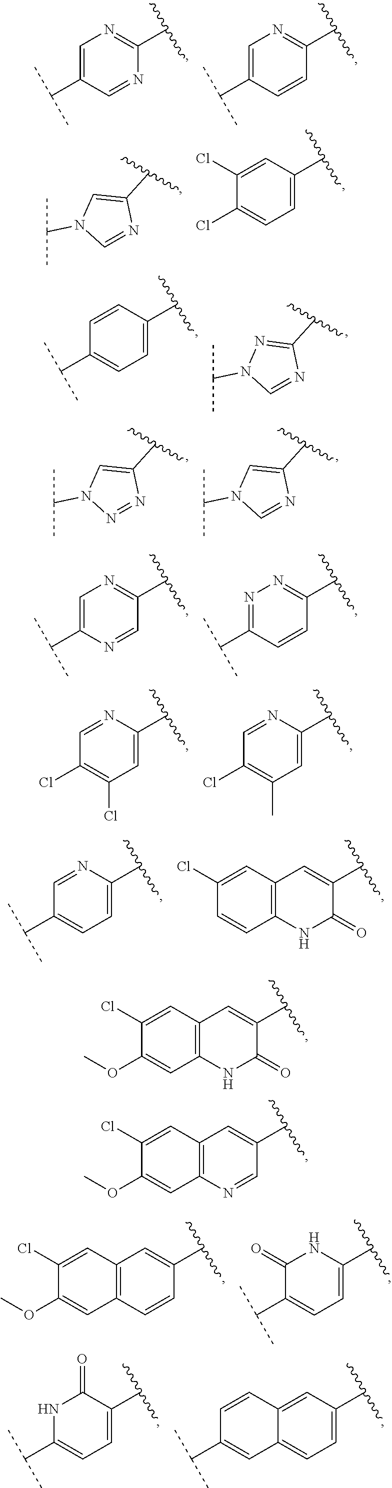 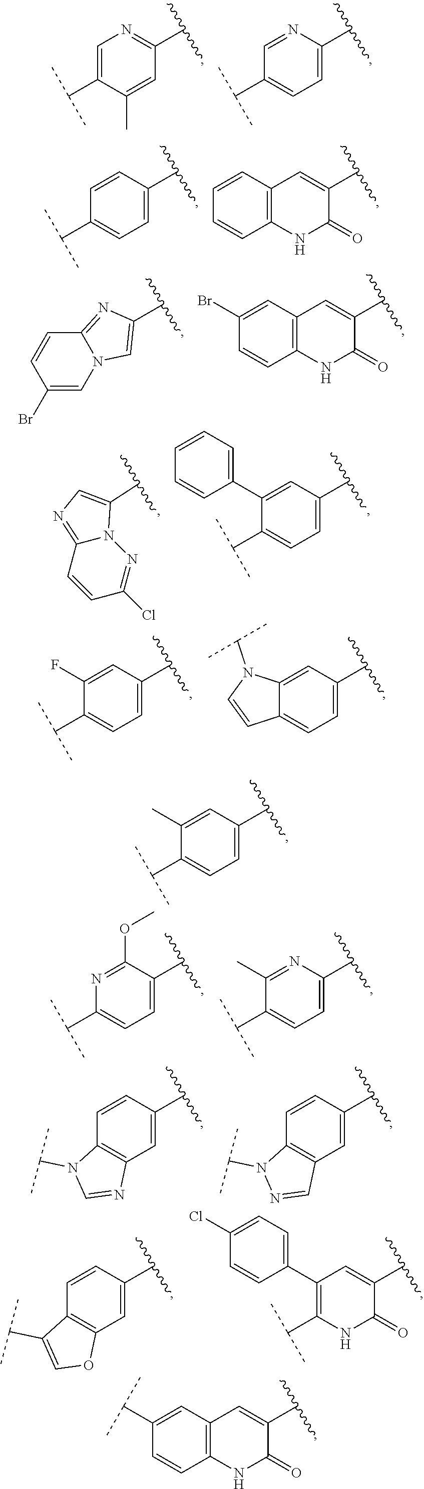 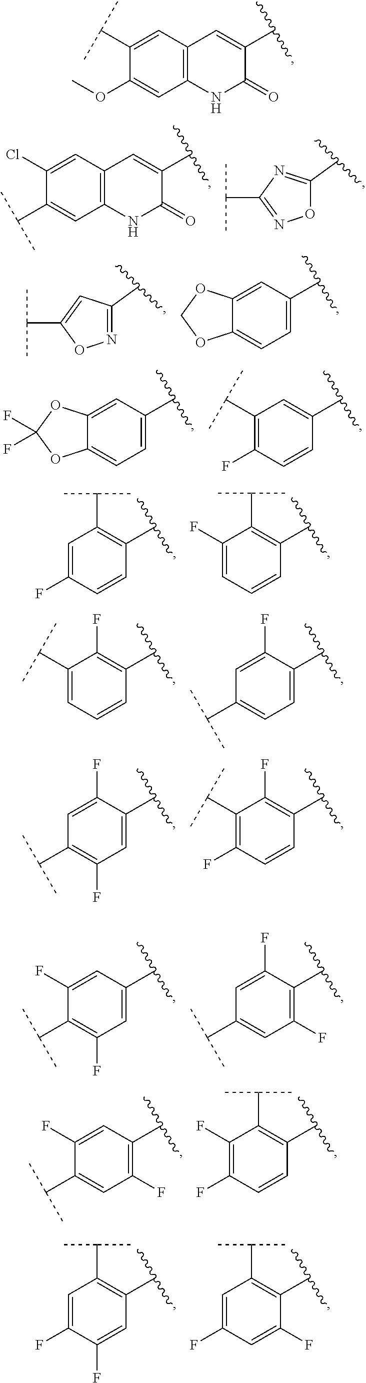 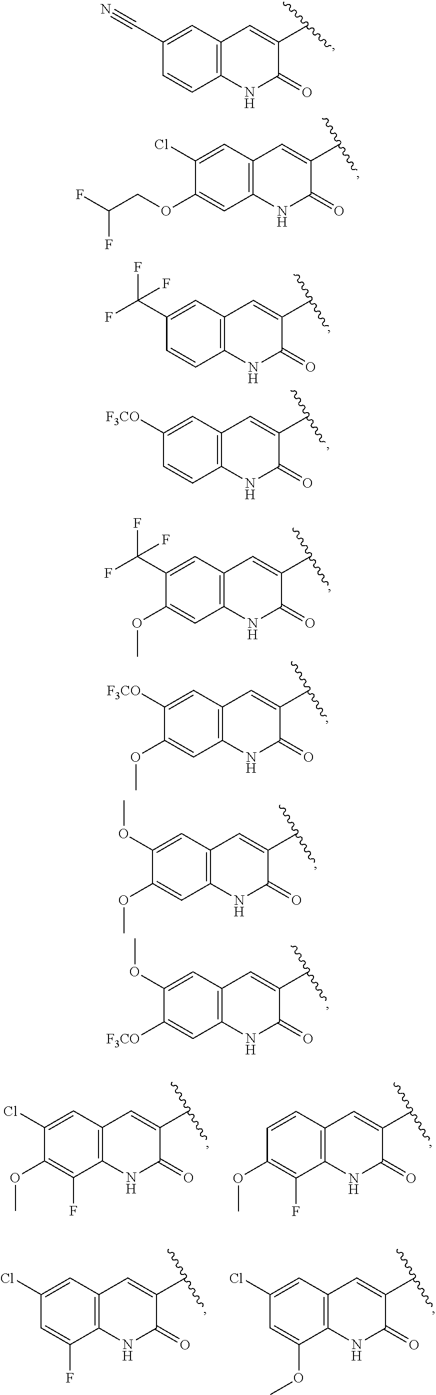 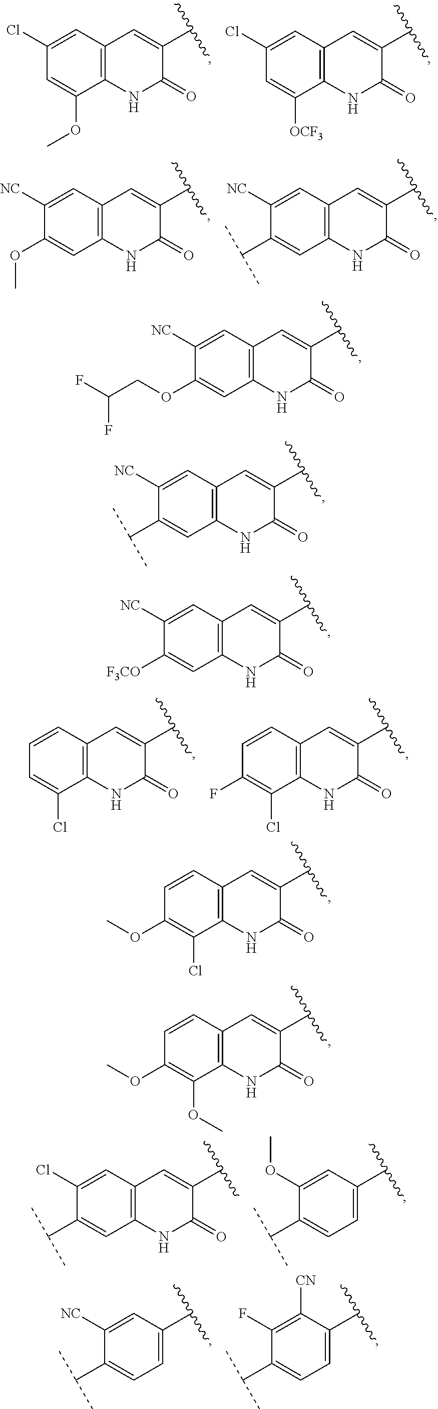 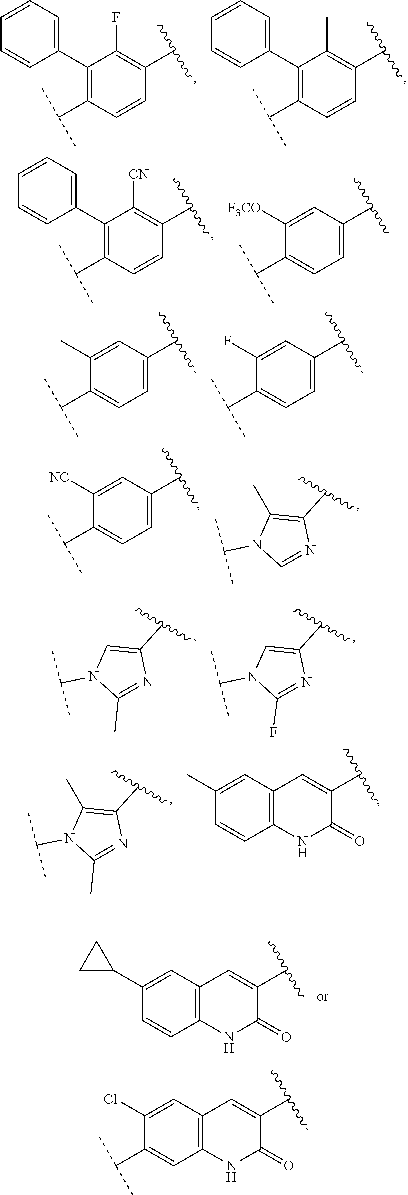
wherein wavy line indicates attachment points to the alkylamine and dotted line indicates attachment points to the L.

14. The compound of claim 1, wherein B is selected from hydrogen, C₆-C₁₀ aryl, 5- to 10-membered heteroaryl, C₃-C₈ cycloalkyl or 3- to 10-membered heterocyclyl each of which is optionally substituted by R^{6b}.

15. The compound of claim 1, wherein B is selected from hydrogen, 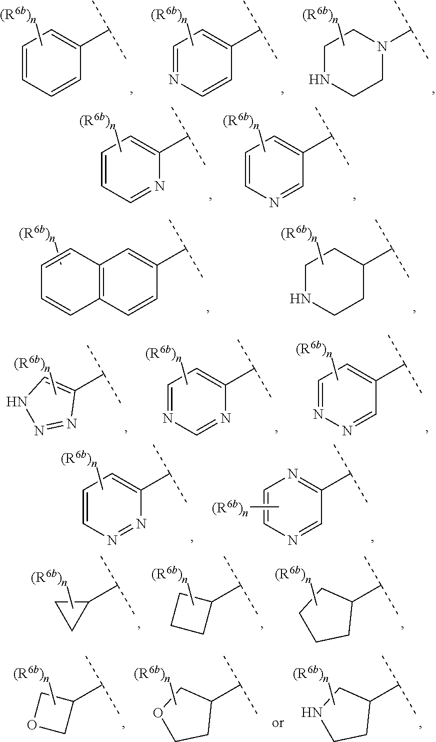
wherein dotted line indicates attachment points to the L.

16. The compound of claim 15, wherein n is 0, 1, 2, 3 or 4.

17. The compound of claim 1, wherein R^{6b} is selected from halogen, —CN, C₃-C₆ cycloalkyl, C₁-C₆ alkyl, C₁-C₆ alkoxy, C₁-C₆ haloalkoxy, C₁-C₆ haloalkyl, —C(O)R¹³ or —OR¹³.

18. The compound of claim 17, wherein R^{6b} is selected from oxo, —CN, —Cl, —F, methyl, —OCH₃, —CF₃, —OCF₃, —C(O)CH₃, —C(O)CH═CH₂ or cyclopropyl.

19. The compound of claim 1, wherein B optionally substituted with R^{6b} is selected from 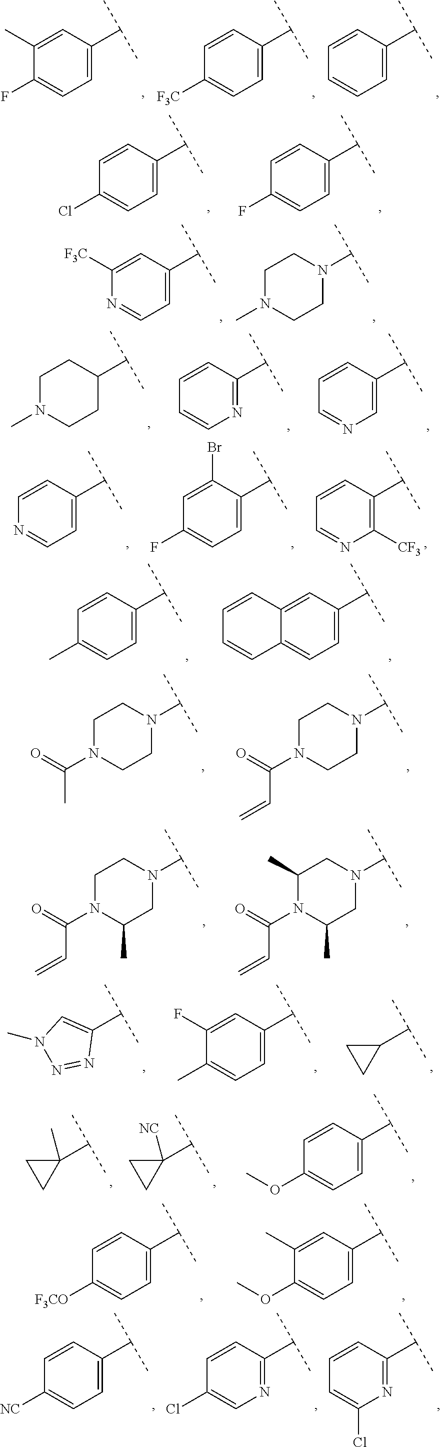 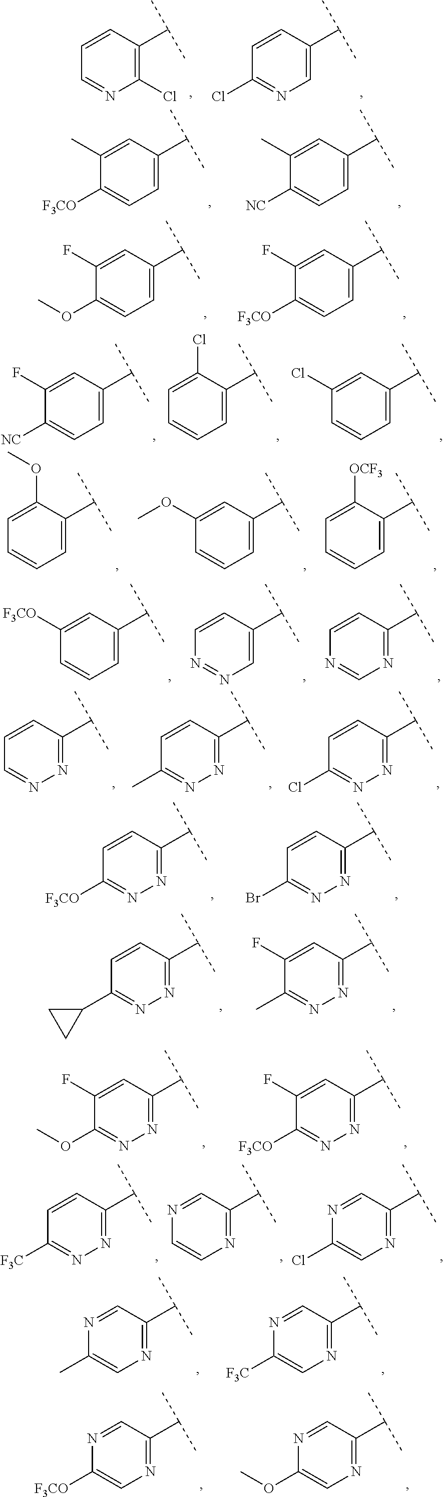 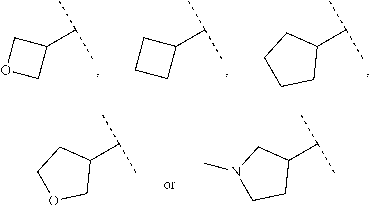
wherein the dotted line denotes attachment point.

20. The compound of claim 1, wherein L is a selected from a bond, —O—, —(CH₂)₁₋₃—, —NH—, —C(O)—, —CH₂—O—, —S—, —CR^{b}R^{c}—, —C(O)NH— or —NHC(O)—.

21. The compound of claim 20, wherein L is selected from a bond, —O—, —CH₂—, —CH₂—O—, —S—, —NH—, —CH(C₂H₅)—, CH(CH₂(cyclopropyl))-, —CF₂— or —C(O)—.

22. The compound of claim 20, wherein L is a bond.

23. The compound of claim 20, wherein L is —O—.

24. The compound of claim 1, wherein A, B, L, R^{6a′} R^{6b}, m and n together are selected from

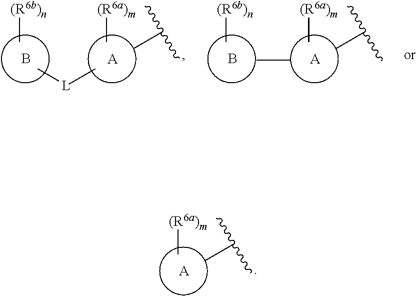

25. The compound of claim 1, wherein A, B, L, R^{6a′} R^{6b}, m and n together are selected from 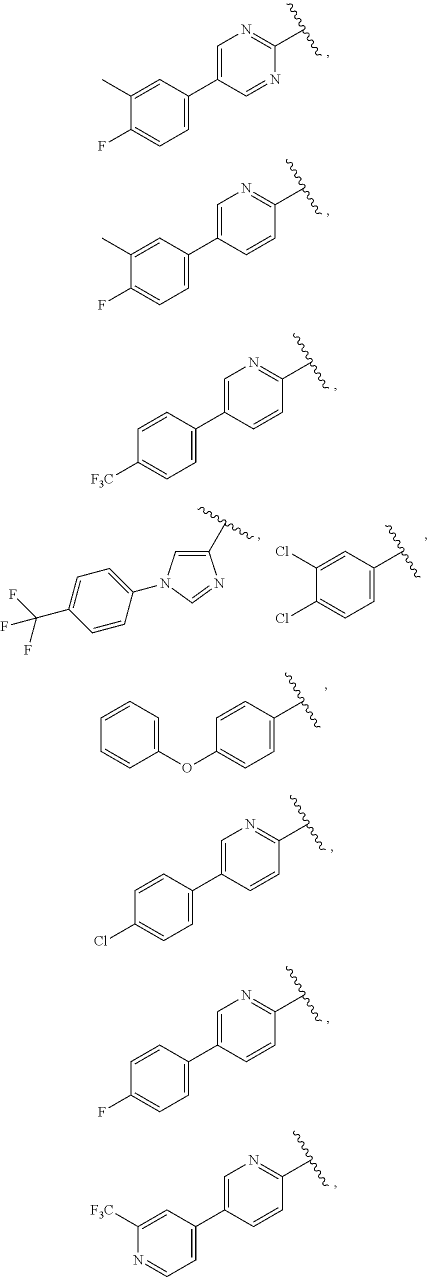 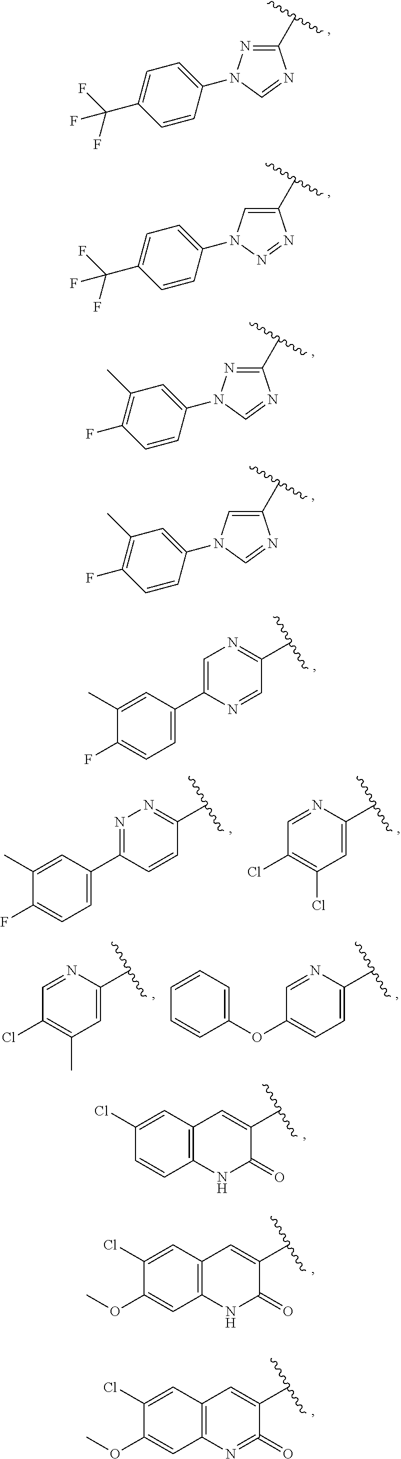 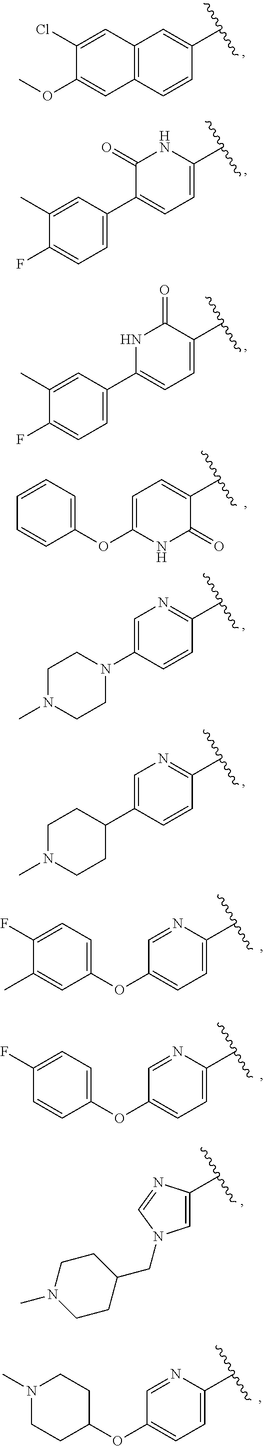 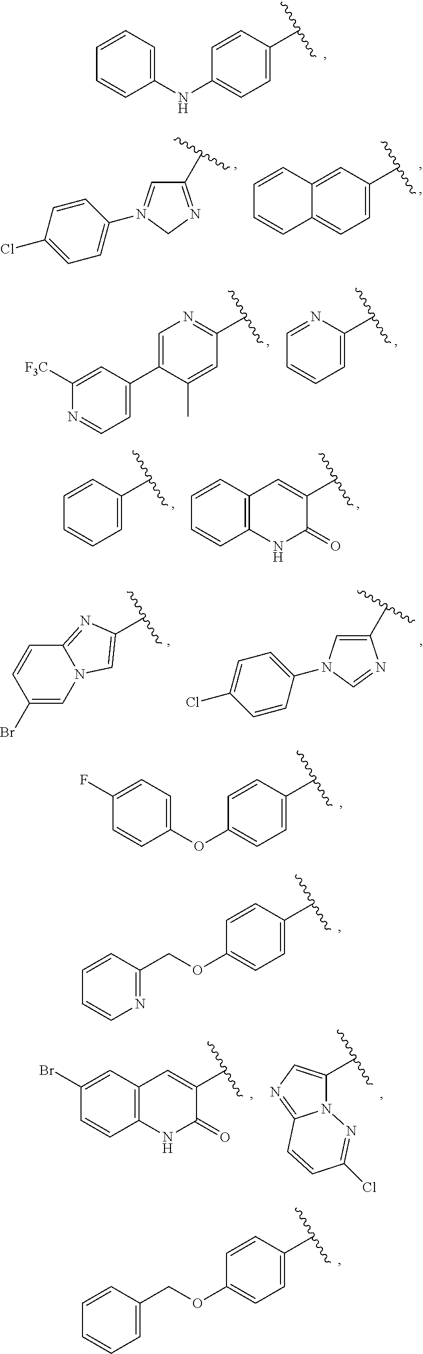 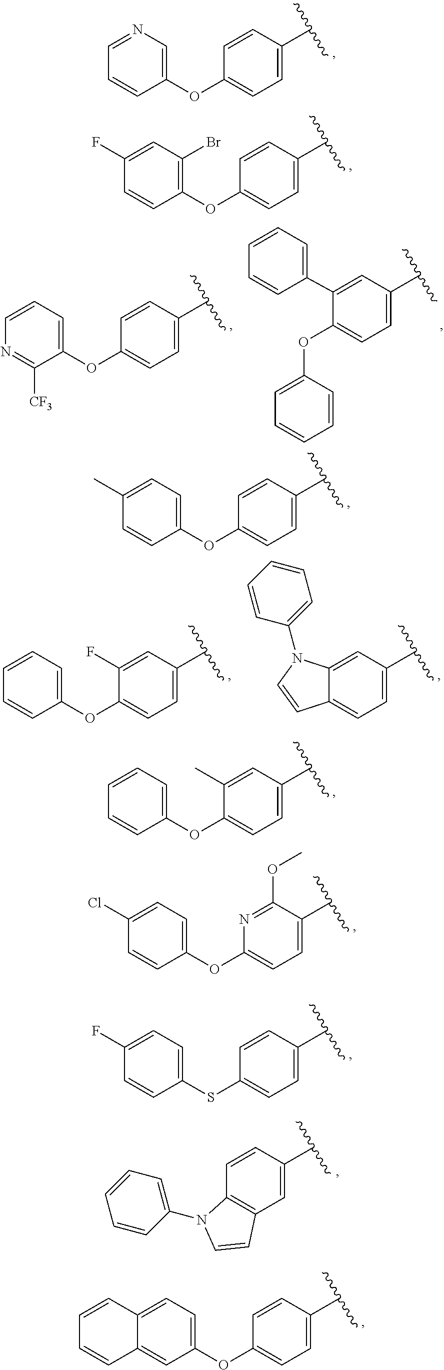 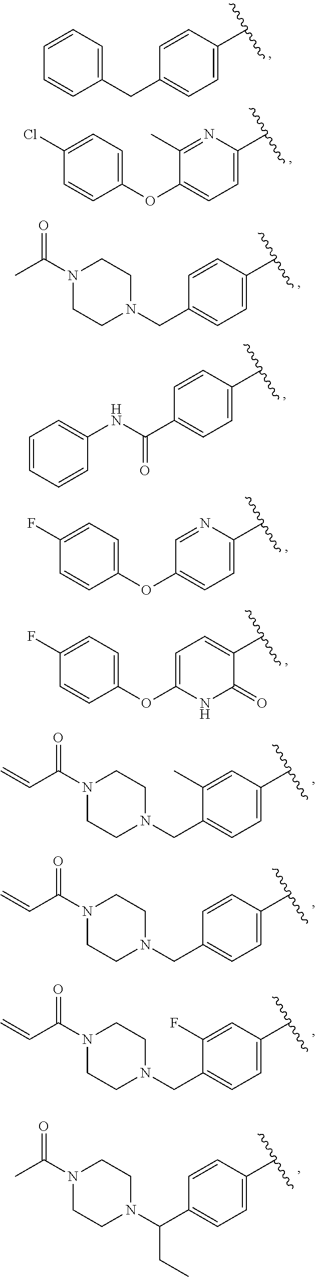 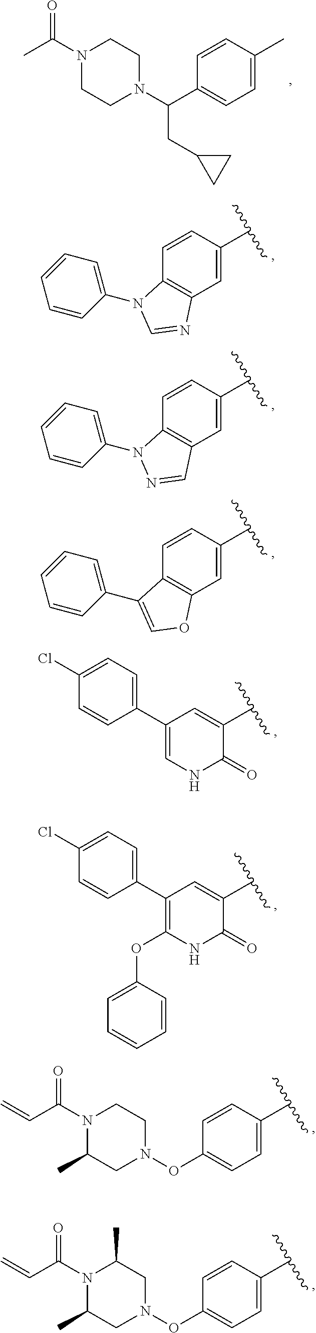 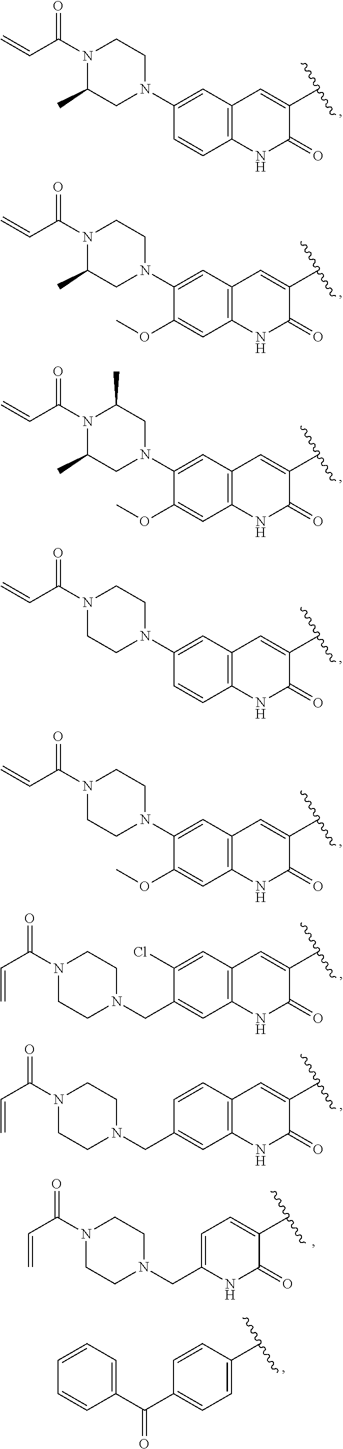 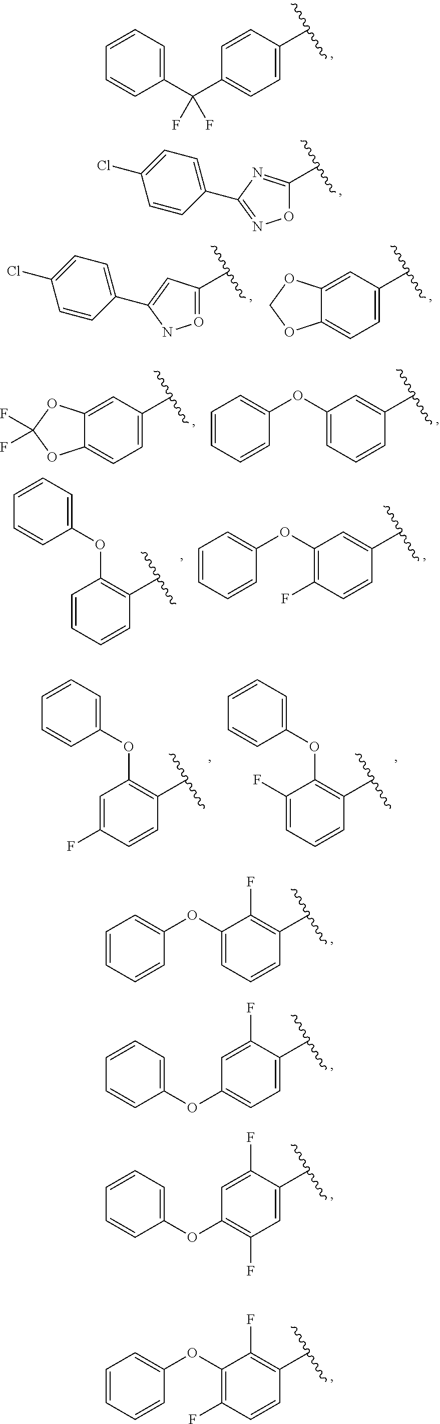 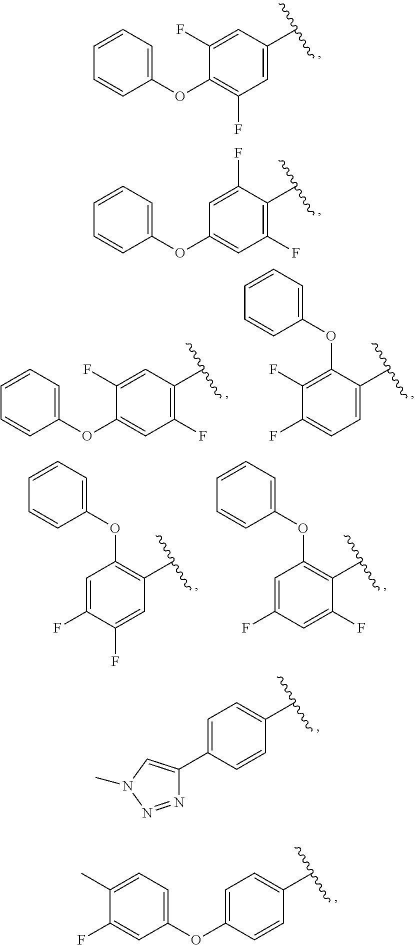 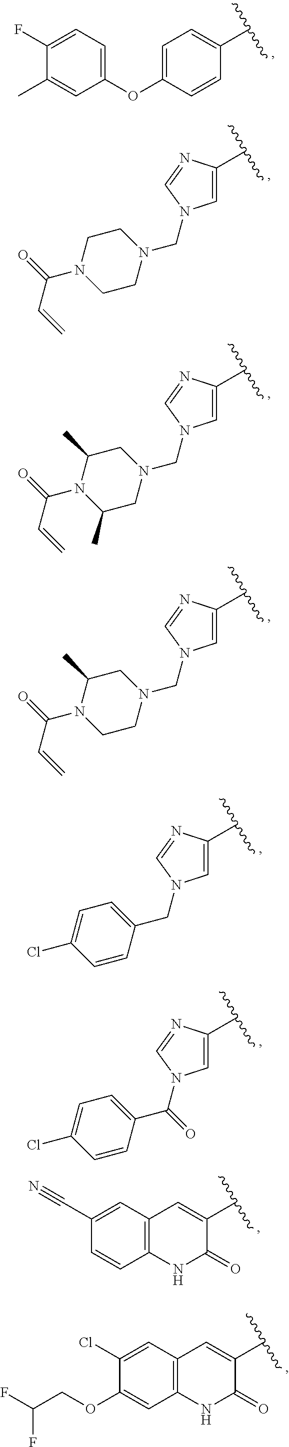 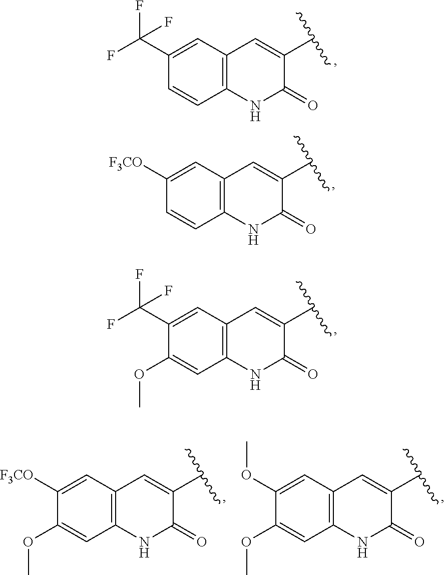 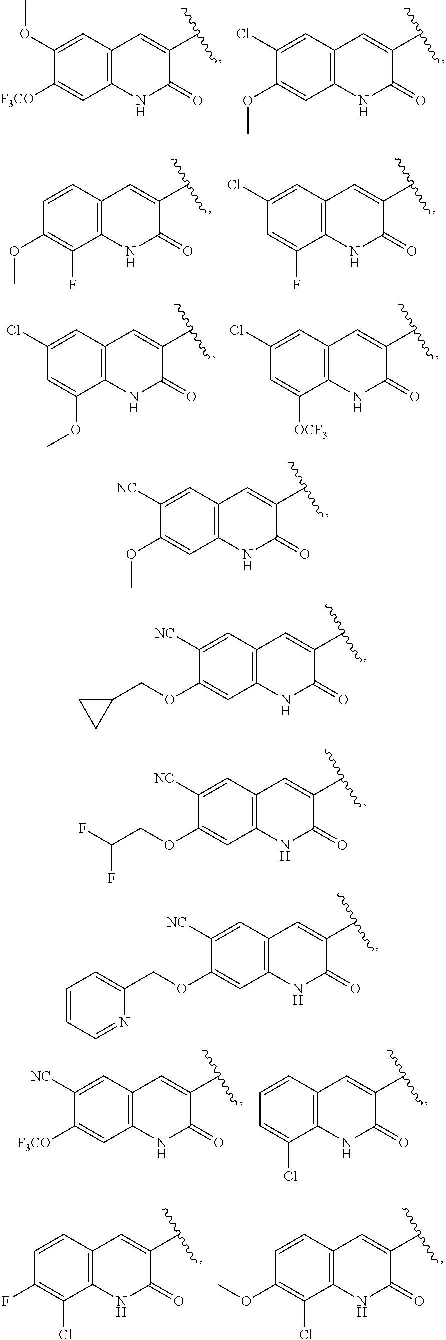 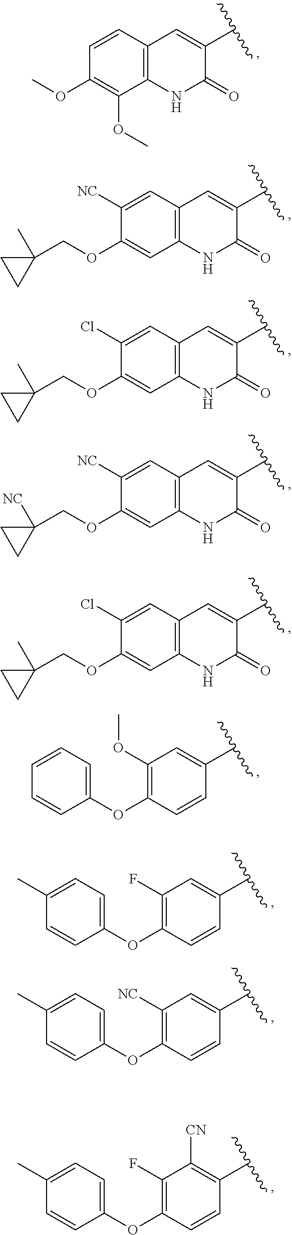 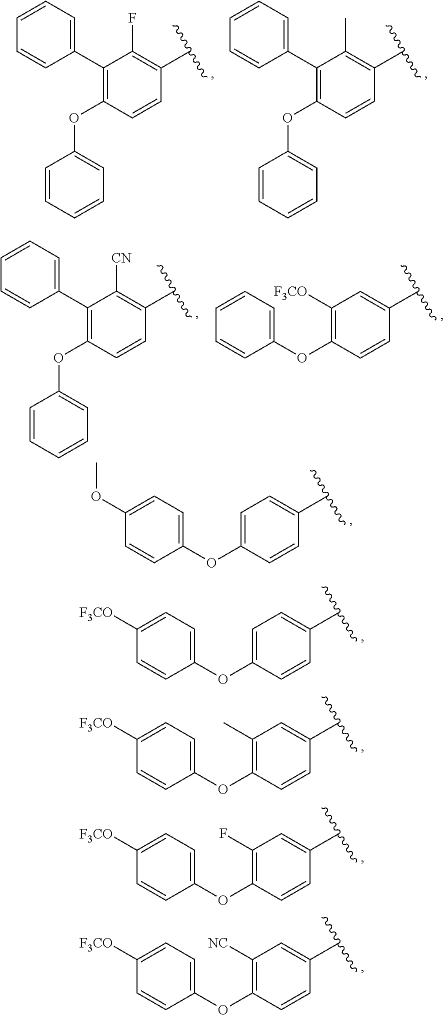 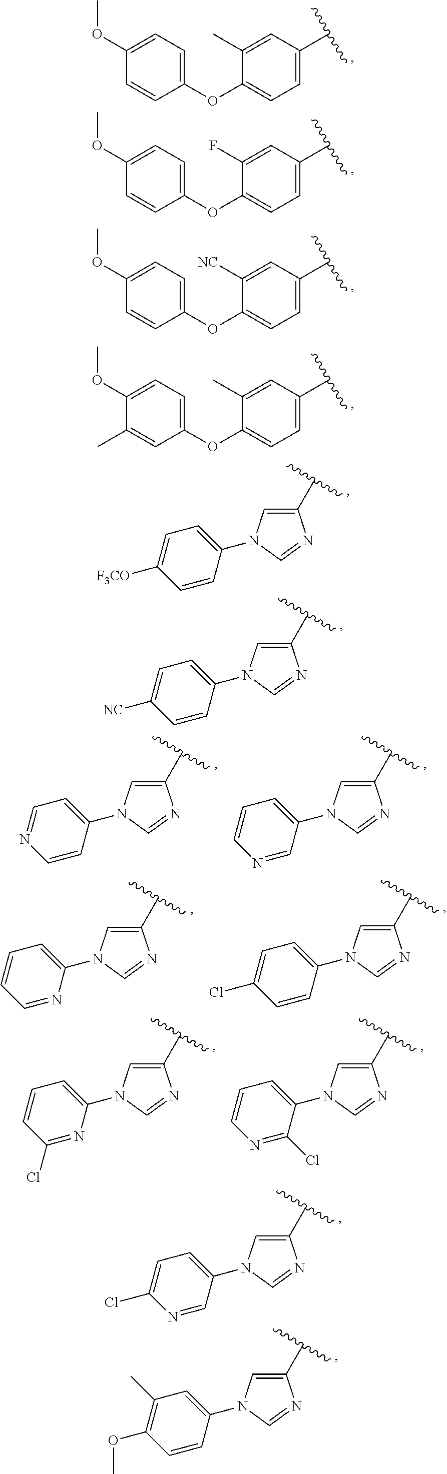 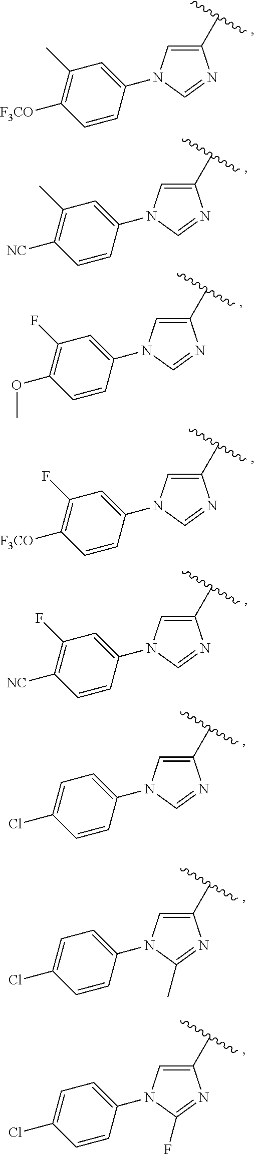 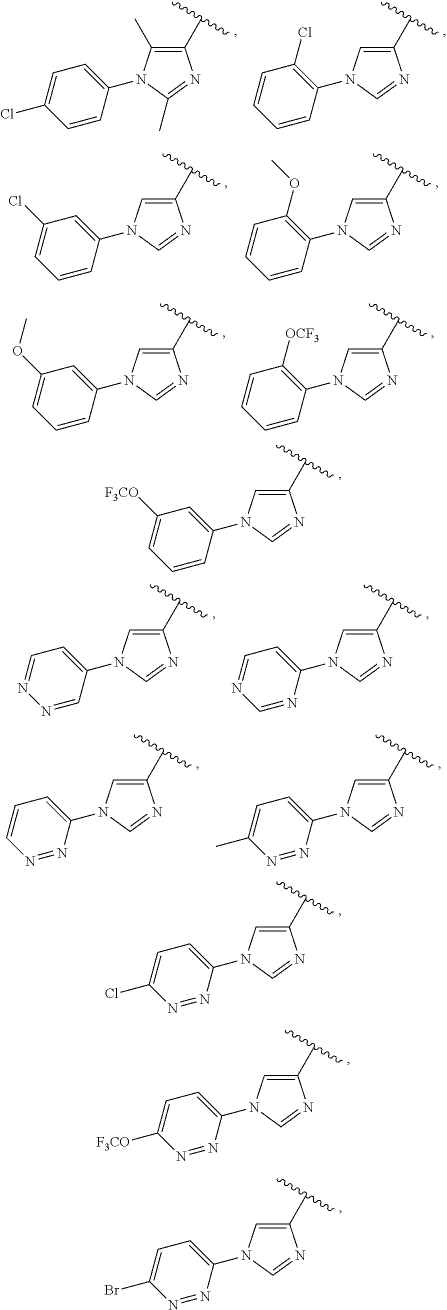 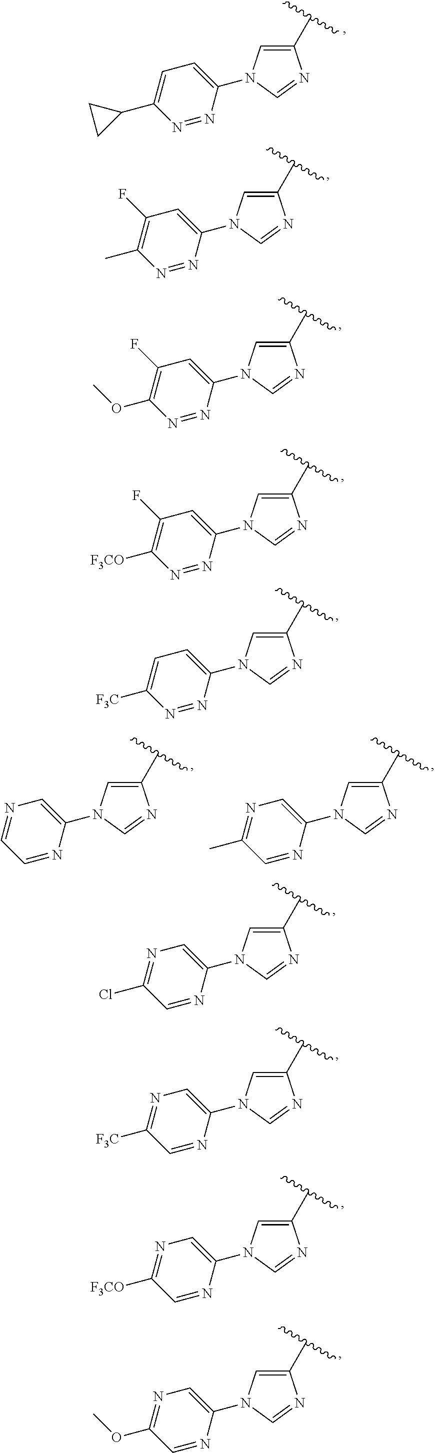 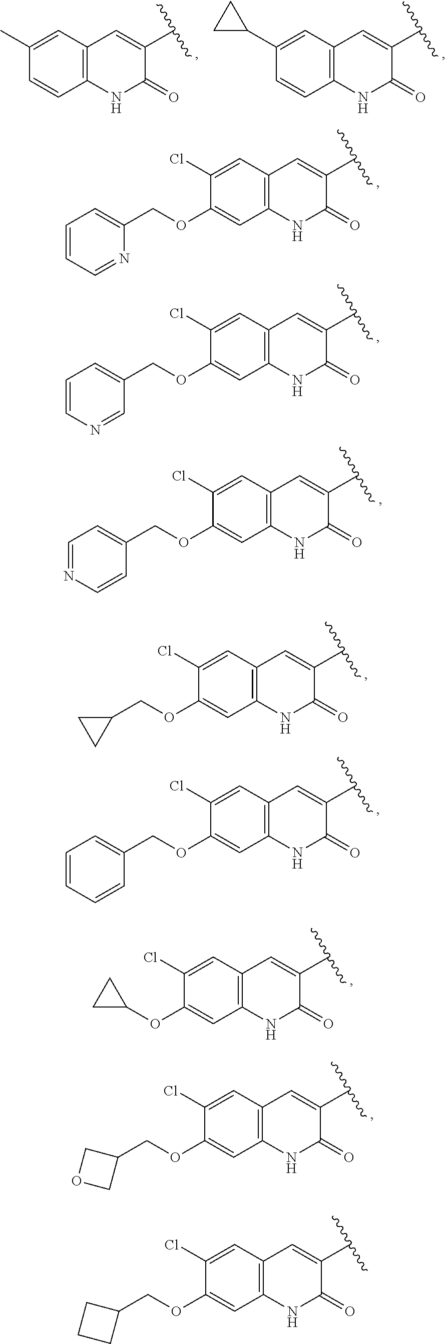 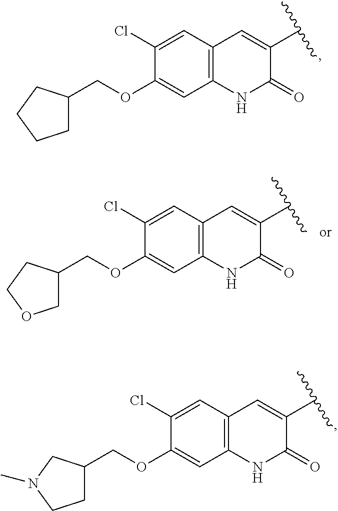 [SMILES: *c1cc2cc(Cl)c(OCC3CCCC3)cc2[nH]c1=O ; *c1cc2cc(Cl)c(OCC3CCOC3)cc2[nH]c1=O ; *c1cc2cc(Cl)c(OCC3CCN(C)C3)cc2[nH]c1=O]
wherein the wavy line denotes attachment point.

26. The compound of claim 1, wherein R¹ is selected from the group consisting of hydrogen, halogen or C₁-C₆ alkyl.

27. The compound of claim 26, wherein R¹ is selected from hydrogen or methyl.

28. The compound of claim 1, wherein R² is selected from hydrogen, halogen, C₁-C₆ alkoxy, C₁-C₆ haloalkoxy, C₃-C₆ cycloalkyl, 3- to 6-membered heterocyclyl, C₆ aryl, 5- to 6-membered heteroaryl or C₁-C₆ alkyl optionally substituted by oxo, halogen, —OR^{2a} or —NR^{2a}R^{2b}.

29. The compound of claim 28, wherein R² is selected from hydrogen, methyl, —CF₃, —CHF₂—, ethyl, —CH(CH₃)CF₃, —CH(CH₃)CHF₂, —CH(CH₂F)₂, —C₂H₄F, isopropyl, isobutyl, —OCH₃, —OCH₂CH₃, —OCH(CH₃)₂, —OC(CH₃)₃, —CH(OH)CH₃, —NH₂, CH(CH₃)NH₂, —CH(CH₃)NHCH₃, —CH(CH₃)—N(CH₃)₂, —CH(CH₃)CH₂F, cyclopropyl, phenyl, —(CH₂)phenyl, 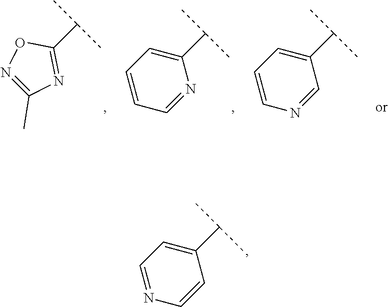
wherein the dotted line denotes the attachment point.

30. The compound of claim 28, wherein R² is selected from ethyl or isopropyl.

31. The compound of claim 1, wherein R¹ and R² are taken together with the atom to which they are attached to form a C₃-C₆ cycloalkyl or 3-6 membered heterocyclyl, each of which is optionally substituted by oxo, —OH, halogen, —NH₂, or C₁-C₆ alkyl optionally substituted by oxo, —OH, halogen or —NH₂.

32. The compound of claim 31, wherein R¹ and R² are taken together with the atom to which they are attached to form cyclopropyl or cyclobutyl.

33. The compound of claim 1, wherein R³ and R⁴ are independently hydrogen, halogen, or C₁-C₆ alkyl optionally substituted by oxo, —OH or halogen.

34. The compound of claim 33, wherein R³ and R⁴ are hydrogen.

35. The compound of claim 1, wherein R³ and R⁴ are taken together with the atom to which they are attached to form a C₃-C₆ cycloalkyl or 3-6 membered heterocyclyl, each of which is optionally substituted by oxo, —OH, halogen, —NH₂, or C₁-C₆ alkyl optionally substituted by oxo, —OH, halogen or —NH₂.

36. The compound of claim 35, wherein R³ and R⁴ are taken together with the atom to which they are attached to form cyclopropyl or oxytanyl ring.

37. The compound of claim 1, wherein R⁵ is selected from hydrogen, C₁-C₆ alkyl, C₂-C₆ alkenyl, C₂-C₆ alkynyl, C₃-C₆ cycloalkyl, 3- to 6-membered heterocyclyl, C₆-aryl, C₅-C₆ heteroaryl, —CN, halogen, C₁-C₆ alkoxy, C₁-C₆ haloalkoxy, —OR¹⁰, —SR¹⁰, —S(O)₂R¹⁰, —S(O)₂NR¹¹R¹², —NR¹⁰S(O)₂R¹¹, —NR¹¹R¹², —C(O)R¹⁰, —NR¹⁰C(O)R¹¹, —NR¹⁰C(O)NR¹¹R¹², —C(O)OR¹⁰, —C(O)ONR¹¹R¹², —C(O)NR¹¹R¹², wherein each of which is optionally substituted by R⁸.

38. The compound of claim 37, wherein R⁵ is selected from hydrogen, methyl, ethyl, ter-butyl, iso-butyl, cyclopropyl, phenyl, —CN, —N(CH₃)₂, —Cl, —Br, —OCH₃, —OC₃H₇, —OCH(CH₃)₂, —OCF₃, —CF₃, —SCH₃, aziridinyl, piperidinyl, propyne, —SO₂NHCH₃, —C(O)OCH₃, isopropene or thiazolyl.

39. The compound of claim 37, wherein R⁵ is hydrogen or methyl.

40. The compound of claim 1, wherein R⁷ and R^{7′} are independently selected from hydrogen, C₃-C₆ cycloalkyl or C₁-C₆ alkyl optionally substituted by halogen or —OH.

41. The compound of claim 40, wherein R⁷ and R^{7′} are independently selected from hydrogen, —CH₃, ethyl, isopropyl, n-propyl, ter-butyl, cyclopropyl, cyclobutyl or —CH₂F.

42. The compound of claim 40, wherein R⁷ is hydrogen and R^{7′} is —CH₃.

43. The compound of claim 1, wherein R⁷ and R^{7′} are taken together with the atom to which they are attached to form a C₃-C₆ cycloalkyl;

44. The compound of claim 43, wherein R⁷ and R^{7′} are taken together with the atom to which they are attached to form a cyclopropyl.

45. The compound of claim 1, wherein the compound is a compound of formula (I), 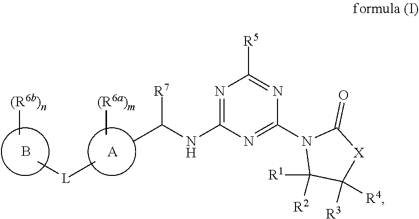
or a salt, polymorph, solvate, enantiomer, stereoisomer or tautomer thereof, wherein X, A, B, L, R¹, R², R³, R⁴, R⁵, R^{6a}, R^{6b}, R⁷, m and n are as defined in claim 1.

46. The compound of claim 1, wherein the compound is any of the compounds of formula (Ia-1) to (Ia-14), 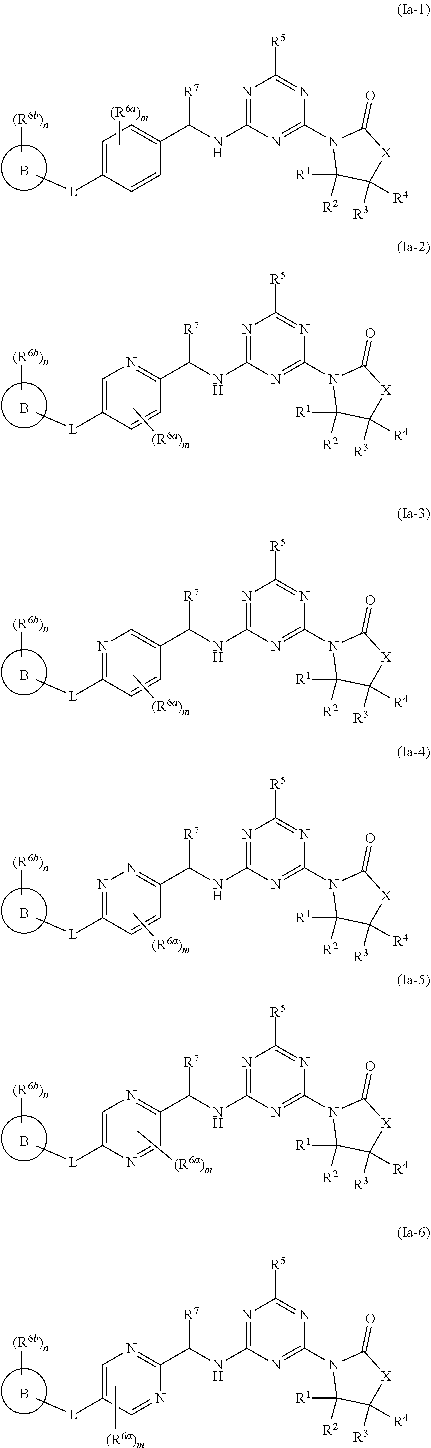 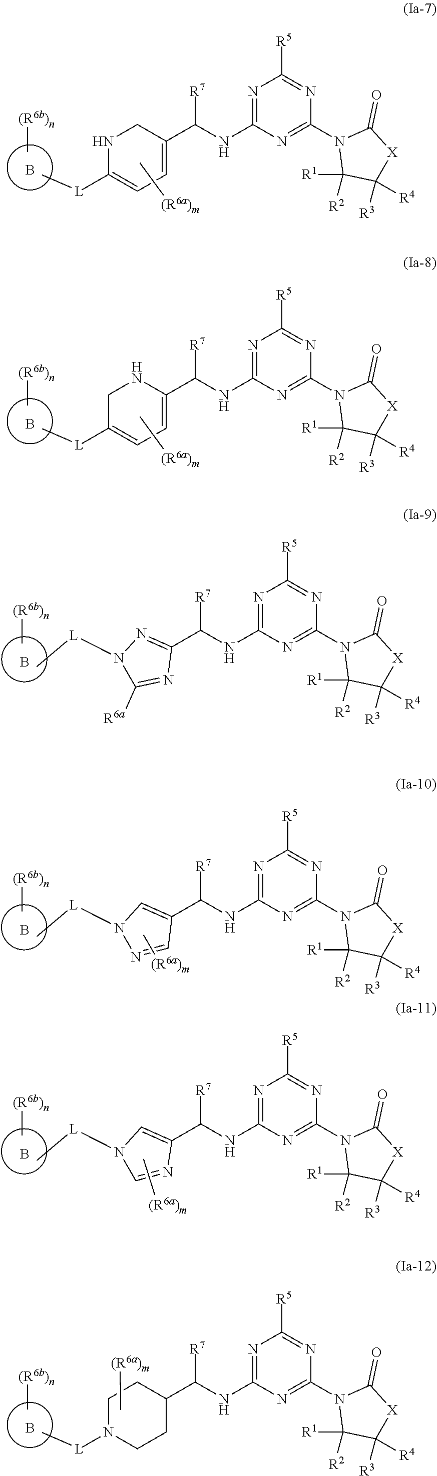 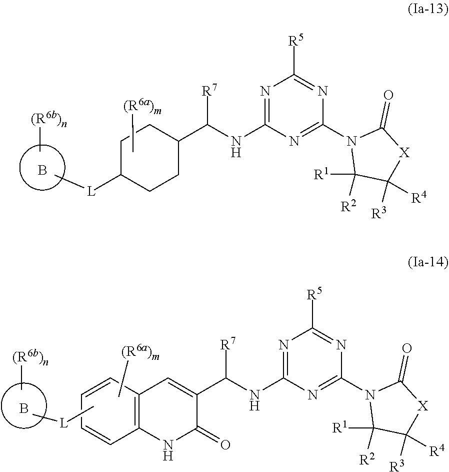
or a salt, polymorph, solvate, enantiomer, stereoisomer or tautomer thereof, wherein B, X, L, R¹, R², R³, R⁴, R⁵, R⁷, R^{6a}, R^{6b}, m and n are as defined in claim 1.

47. The compound of claim 1, wherein the compound is any of the compounds of formula (Ib-1) to (Ib-11), 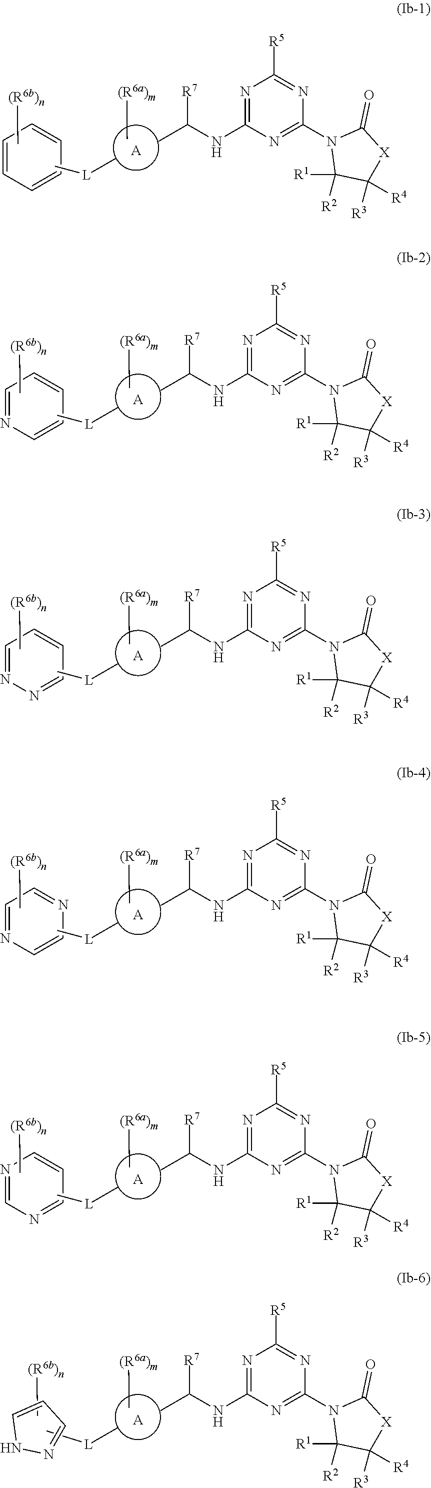 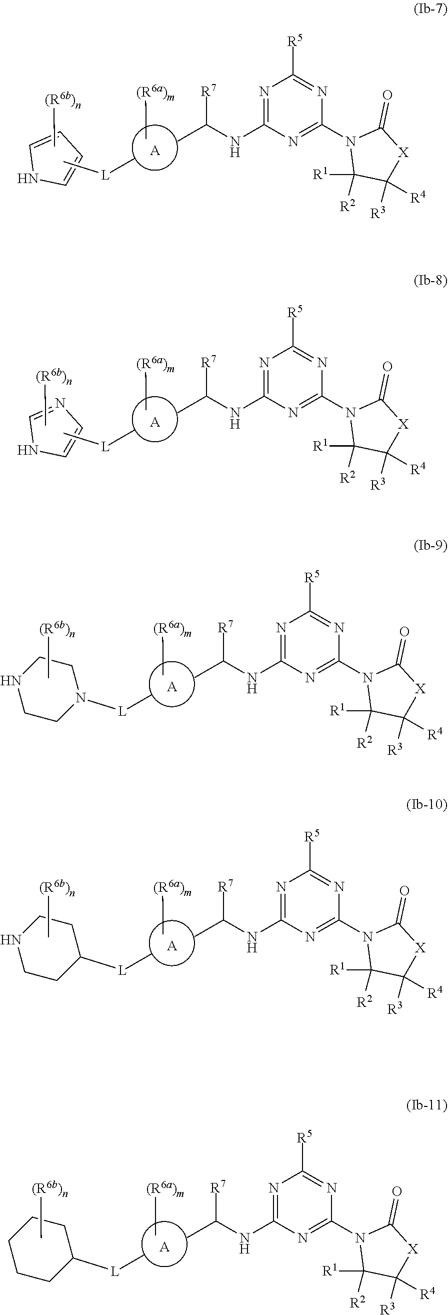
or a salt, polymorph, solvate, enantiomer, stereoisomer or tautomer thereof, wherein A, X, L, R¹, R², R³, R⁴, R⁵, R⁷, R^{6a}, R^{6b}, m and n are as defined in claim 1.

48. The compound of claim 1, wherein the compound is a compound of formula (II): 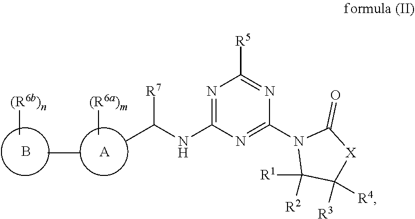
or a salt, polymorph, solvate, enantiomer, stereoisomer or tautomer thereof, wherein A, B, X, R¹, R², R³, R⁴, R⁵, R⁷, R^{6a}, R^{6b}, m and n are as defined in claim 1.

49. The compound of claim 1, wherein the compound is any of the compounds of formula (IIa-1) to (IIa-8), 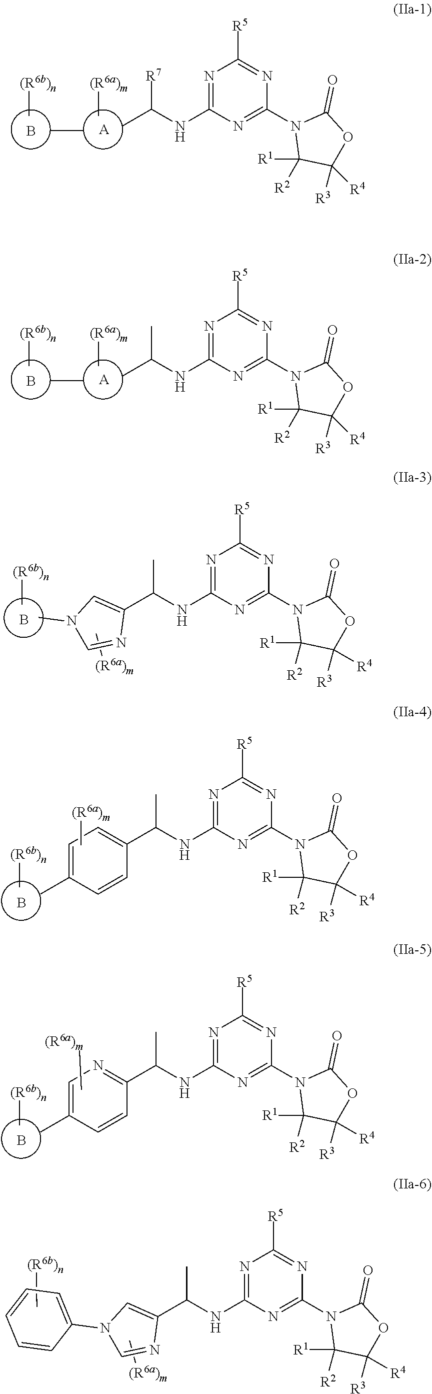 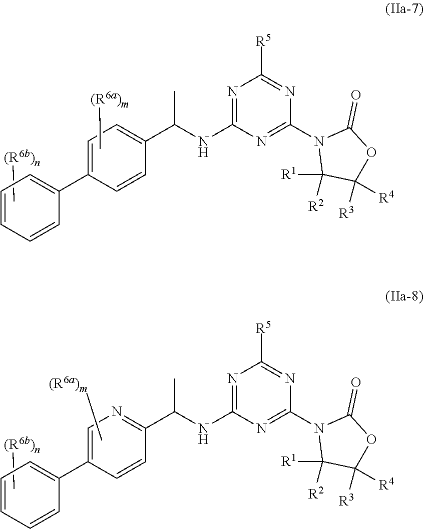
or a salt, polymorph, solvate, enantiomer, stereoisomer or tautomer thereof, wherein A, X, L, R¹, R², R³, R⁴, R⁵, R⁷, R^{6a}, R^{6b}, m and n are as defined in claim 1.

50. The compound of claim 1, wherein the compound is a compound of formula (III): 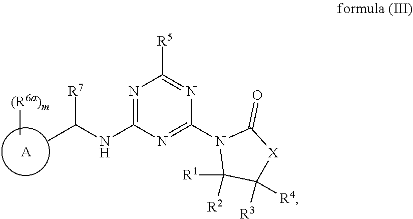
or a salt, polymorph, solvate, enantiomer, stereoisomer or tautomer thereof, wherein A, X, R¹, R², R³, R⁴, R⁵, R⁷, R^{6a} and m are as defined in claim 1.

51. The compound of claim 1, wherein the compound is any of the compounds of formula (IIIa-1) to (IIIa-8), 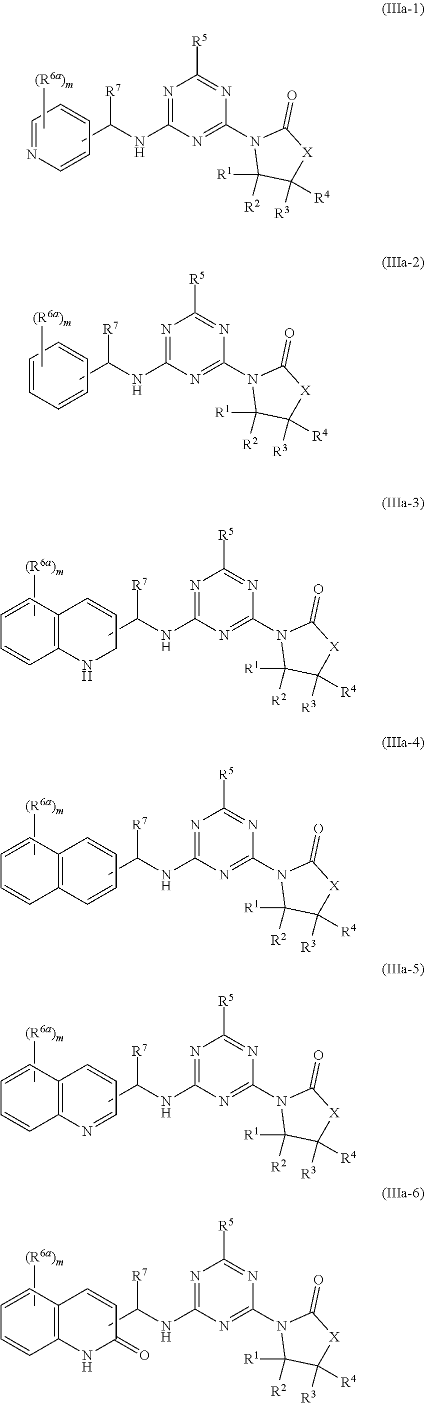 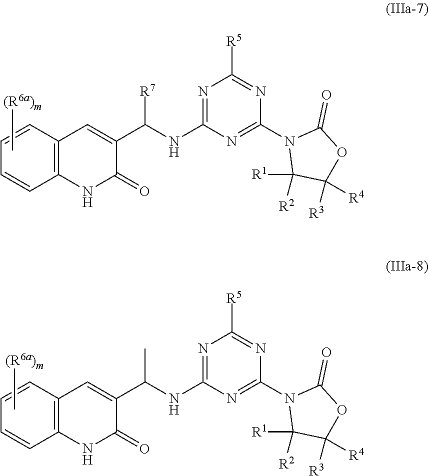
or a salt, polymorph, solvate, enantiomer, stereoisomer or tautomer thereof, wherein X, R¹, R², R³, R⁴, R⁵, R⁷, R^{6a} and m are as defined in claim 1.

52. The compound of claim 1, wherein the compound is a compound of formula (IV), 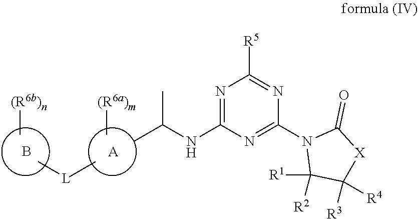
or a salt, polymorph, solvate, enantiomer, stereoisomer or tautomer thereof, wherein X, R¹, R², R³, R⁴, R⁵, R⁷, R^{6a} and m are as defined in claim 1.

53. The compound of claim 1, wherein the compound is any of the compounds of formula (IVa-1) to (IVa-7), 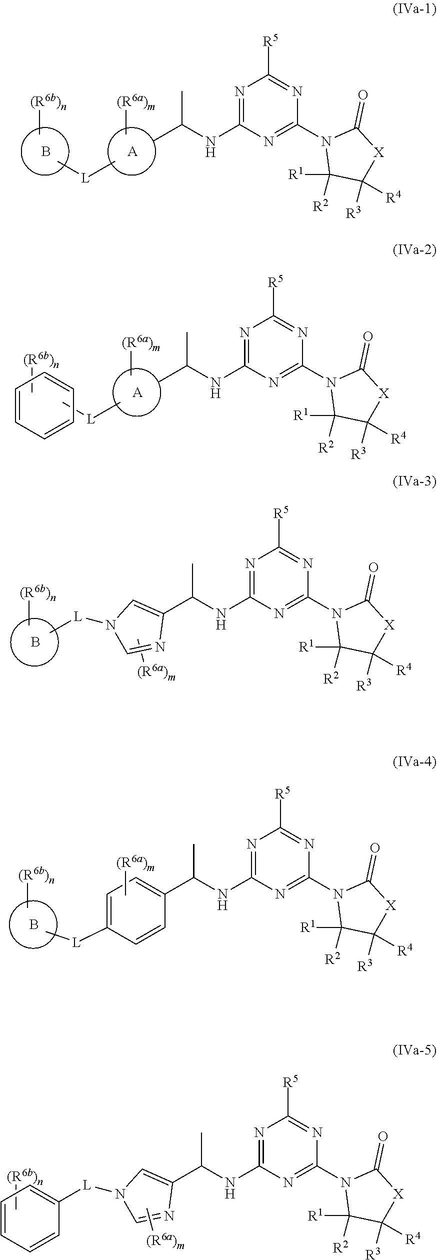 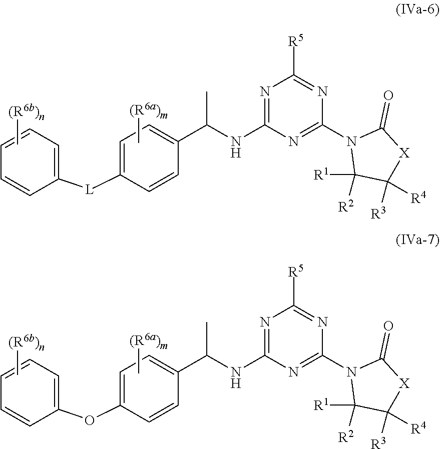
or a salt, polymorph, solvate, enantiomer, stereoisomer or tautomer thereof, wherein X, R¹, R², R³, R⁴, R⁵, R⁷, R^{6a} and m are as defined in claim 1.

54. The compound of claim 1, wherein the compound is a compound of formula (V), 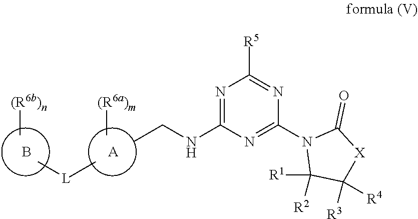
or a salt, polymorph, solvate, enantiomer, stereoisomer or tautomer thereof, wherein X, R¹, R², R³, R⁴, R⁵, R⁷, R^{6a} and m are as defined in claim 1.

55. The compound of claim 1, wherein the compound is a compound of formula (VI), 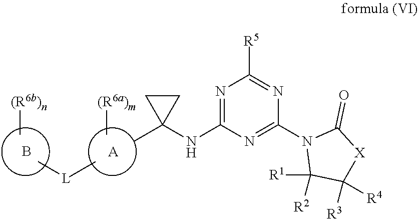
or a salt, polymorph, solvate, enantiomer, stereoisomer or tautomer thereof, wherein X, R¹, R², R³, R⁴, R⁵, R⁷, R^{6a} and m are as defined in claim 1.

56. The compound of claim 1, wherein the compound is selected from Compound Nos. 1.1 to 1.113 in table 1 or a salt, polymorph, solvate, enantiomer, stereoisomer or tautomer thereof.

57. The compound of claim 1, wherein the compound is selected from Compound Nos. 2.1 to 2.462 in table 2 or a salt, polymorph, solvate, enantiomer, stereoisomer or tautomer thereof.

58. A pharmaceutical composition comprising the compound of claim 1, or a salt, polymorph, solvate, enantiomer, stereoisomer or tautomer thereof, and a pharmaceutically acceptable carrier.

59. A method of treating disease associated with mutant IDH in an individual in need thereof comprising administering to the individual a therapeutically effective amount of the compound of claim 1, or a pharmaceutically acceptable salt thereof.

60. The method of treating of claim 59, wherein the mutant IDH is mutant IDH1.

61. A method of treating cancer in an individual in need thereof comprising administering to the individual a therapeutically effective amount of the compound of claim 1, or a salt, polymorph, solvate, enantiomer, stereoisomer or tautomer thereof.

62. The method of claim 61, further comprising administering to the individual a therapeutically effective amount of other therapeutic agent.

63. A method of inhibiting mutant IDH1 in an individual in need thereof comprising administering the compound of claim 1, or a salt, polymorph, solvate, enantiomer, stereoisomer or tautomer thereof.

64. Use of the compound of claim 1, or a pharmaceutically acceptable salt or solvate thereof, in the manufacture of a medicament for treatment of a disease mediated by a mutant isocitrate dehydrogenase (IDH), preferably mutant IDH1.

65. A kit comprising the compound of claim 1, or a salt, polymorph, solvate, enantiomer, stereoisomer or tautomer thereof.
